(19) United States
(12) Patent Application Publication
(10) Pub. No.: US 2026/0114178 A1
(43) Pub. Date: Apr. 23, 2026

(54) ORGANIC MATERIAL COMPOSITION AND USE THEREOF

(71) Applicant: Ningbo Lumilan Advanced Materials Co., Ltd., Ningbo (CN)

(72) Inventors: Cong Tang, Ningbo (CN); Dingwei Wei, Ningbo (CN); Fengjiao Liu, Ningbo (CN); Huanda Ding, Ningbo (CN); Yang Xiao, Ningbo (CN); Zhikuan Chen, Ningbo (CN)

(73) Assignee: Ningbo Lumilan Advanced Materials Co., Ltd., Ningbo (CN)

(21) Appl. No.: 19/154,564

(22) PCT Filed: Nov. 22, 2024
(86) PCT No.: PCT/CN2024/133903

§ 371 Date: Aug. 7, 2025

(30) Foreign Application Priority Data
Mar. 28, 2024 (CN) 202410371385.6

(51) Int. Cl. H10K 85/60; C07D 251/24; C07D 401/10; C07D 405/04; C07D 413/12; C09K 11/02; H10K 50/11

(52) U.S. Cl. CPC H10K 85/636; C07D 251/24; C07D 401/10; C07D 405/04; C07D 413/12; C09K 11/02; H10K 85/626; H10K 85/633; H10K 85/654; H10K 85/6572; H10K 50/11; H10K 85/615; H10K 85/657; H10K 85/6574

(57) ABSTRACT

The present application relates to the field of display technology, and in particular to an organic material composition and use thereof. This organic material composition comprises a first compound and a second compound. The first compound and the second compound have the following structures:

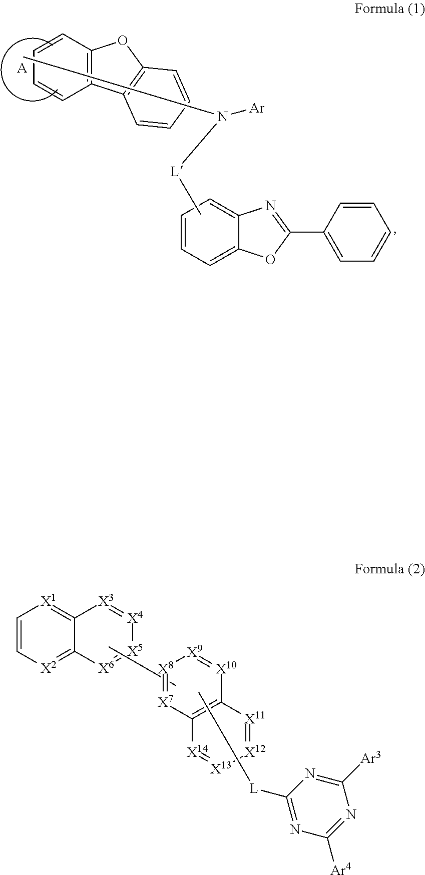

the first compound having a structure as shown in Formula (1) and the second compound having a structure as shown in Formula (2) cooperate with each other, which is beneficial for matching HOMO and LUMO energy levels with adjacent energy levels, so that the organic material composition obtains higher stability and more balanced carrier mobility, thereby making the organic electroluminescent device containing the material have a better lifespan, and also has a lower driving voltage and higher efficiency.

### CROSS-REFERENCE TO RELATED APPLICATION

The present application claims priority to Chinese Patent Application No. 202410371385.6, filed to the China National Intellectual Property Administration on Mar. 28, 2024, entitled “ORGANIC MATERIAL COMPOSITION AND USE THEREOF”, the entire contents of which are incorporated herein by reference.

### TECHNICAL FIELD

The present application relates to the field of display technology, and in particular to an organic material composition and application thereof.

### BACKGROUND

Organic electroluminescent devices (OLED) are devices that convert electrical energy into light by applying electricity to organic electroluminescent materials, and generally have a structure that comprises an anode, a cathode, and an organic layer disposed between the anode and the cathode. The organic layer of an organic EL device may a hole injection layer, a hole transport layer, a hole-assisting layer, an emission-assisting layer, an electron blocking layer, a light-emitting layer (containing a host material and a dopant material), an electron buffer layer, a hole blocking layer, an electron transport layer, an electron injection layer, and so on. Moreover, the materials used for the organic layer are classified by their functions as hole transport materials, hole assisting materials, luminescence assisting materials, electron blocking materials, luminescent materials, electron buffer materials, hole blocking materials, electron transport materials, electron injection materials, and so on. In the organic EL device, holes from the anode and electrons from the cathode are injected into the light-emitting layer by application of voltage, and excitons having high energy are generated by recombination of the holes and the electrons. The organic light emitting compound moves to an excited state by energy and emits light by the energy when the organic light emitting compound returns to a ground state from the excited state.

At present, the functional materials composed of existing organic light-emitting compounds have low stability and unbalanced carrier mobility, which causes the organic electroluminescent diodes to have high driving voltage and short lifespan, seriously limiting the use of organic electroluminescent diodes.

### SUMMARY OF THE INVENTION

The object of the present application is to overcome the problems of high driving voltage and short lifespan in organic light-emitting diodes caused by the low stability and unbalanced carrier mobility of the functional materials composed of existing organic light-emitting compounds, which seriously limit the application of organic electroluminescent diodes, and further provide an organic electroluminescent material and use thereof.

In the present application, the definition of substituent terms is as follows.

As used in the present application, the term □halogen□ may include fluorine, chlorine, bromine or iodine.

As used in the present application, the term □C1-C30 alkyl□ refers to a monovalent substituent derived from a straight chain or branched chain saturated hydrocarbon having 1 to 30 carbon atoms, examples of which include but are not limited to methyl, ethyl, propyl, isobutyl, sec-butyl, tert-butyl, pentyl, isopentyl and hexyl.

As used in the present application, the term □C3-C30 cycloalkyl□ refers to a group derived from a monocyclic hydrocarbon or a polycyclic hydrocarbon having 1 to 30 ring main chain carbon atoms, and the cycloalkane may include cyclopropyl, cyclobutyl, adamantyl, and so on.

In the present application, aryl and arylene include monocyclic, polycyclic or fused ring aryl groups, the rings may be interrupted by short non-aromatic units among the rings and may contain spiro structures. Aryl includes, but is not limited to phenyl, biphenyl, terphenyl, naphthyl, phenanthryl, anthryl, fluorenyl, spirobifluorenyl, and so on, and arylene include but are not limited to phenylene, biphenylene, terphenylene, naphthylene, phenanthrene, anthrylene, fluorenylene, spirobifluorenylene groups, and so on.

In the present application, heteroaryl and heteroarylene include monocyclic, polycyclic, or fused ring heteroaryl groups, the rings may be interrupted by short non-aromatic units among the rings, and the heteroatoms include nitrogen, oxygen, and sulfur. Heteroaryl includes, but is not limited to furnan, thiophene, pyrrole, imidazole, pyrazole, thiazole, thiadiazole, isothiazole, isoxazole, oxazole, oxadiazole, triazine, tetrazine, triazole, tetrazole, furazan, pyridine, pyrazine, pyrimidine, pyridazine, benzofuran, benzothiophene, isobenzofuran, dibenzofuran, dibenzothiophene, benzimidazole, benzothiazole, benzisothiazole, benzisoxazole, benzoxazole, isoindole, indole, indazole, benzothiadiazole, quinoline, isoquinoline, cinnoline, quinazoline, quinoxaline, carbazole, phenoxazine, phenothiazine, phenanthridine, benzodioxolane, dihydroacridine, and derivatives thereof. Heteroarylene includes, but is not limited to furylidene, phenylthioylidene, pyrrolylidene, imidazolylidene, pyrazolylidene, thiazolylidene, thiadiazolylidene, isothiazolylidene, isoxazolylidene, oxazolylidene, oxadiazolylidene, triazinlidene, tetrazinylidene, triazolylidene, tetrazolylidene, furazanylidene, pyridylidene, pyrazinylidene, pyrimidinylidene, pyridazinylidene, benzofurylidene, benzothienylidene, isobenzofurylidene, dibenzofurylidene, dibenzothienylidene, benzimidazolylidene, benzothiazolylidene, benzisothiazolylidene, benzisoxazolylidene, benzoxazolylidene, isoindolylidene, indolylidene, indazolylidene, benzothiadiazolylidene, quinolinylidene, isoquinolinylidene, cinnolinylidene, quinazolinylidene, quinoxalinylidene, carbazolylidene, phenoxazinylidene, phenothiazinylidene, phenanthridinylidene, benzodioxolylidene, acridinylidene, and derivatives thereof.

As used in the present application, the term □substituted□ means that a hydrogen atom in a compound is replaced by another substituent. This position is not limited to a specific location as long as the hydrogen at the position can be replaced by a substituent. When two or more substituents are present, the two or more substituents may be the same or different.

As used in the present application, unless otherwise specified, hydrogen atoms include protium, deuterium and tritium.

In the present application, the range of the number of carbon atoms is defined in the definition of the group, and the number of carbon atoms is any integer within the defined range. For example, for C6-C30 aryl, the number of carbon atoms representing the aryl can be any integer within the range of 6-60, such as 6, 8, 10, 13, 15, 17, 20, 22, 25, or 30, and so on.

The technical solutions adopted in the present application are as follows.

The present application provides an organic material composition, which comprises a first compound and a second compound, wherein the first compound has a structure shown in Formula (1):

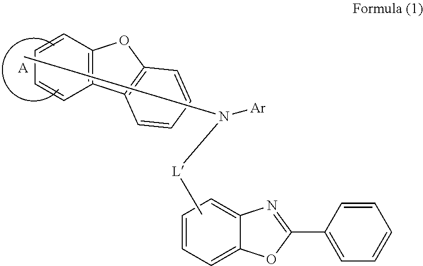

wherein ring A is a benzene ring;
Ar is selected from the group consisting of substituted or unsubstituted C6-C60 aryl, substituted or unsubstituted C6-C60 arylamine, substituted or unsubstituted C3-C60 heteroarylamine, and substituted or unsubstituted C3-C30 heteroaryl;
L′ is selected from the group consisting of substituted or unsubstituted C6-C30 arylene, substituted or unsubstituted C3-C30 heteroarylene;
wherein the second compound has a structure shown in Formula (2):

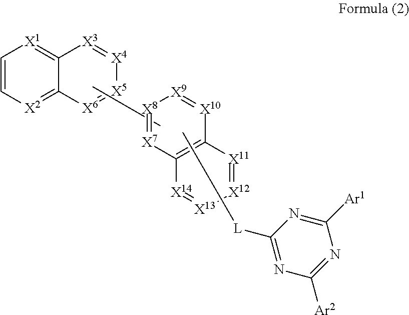

wherein X¹-X¹⁴ are each independently selected from N or CR, and R is selected from the group consisting of hydrogen, deuterium, and C6-C30 aryl;
L is independently selected from the group consisting of a connecting bond, substituted or unsubstituted C6-C30 arylene, and substituted or unsubstituted C3-C30 heteroarylene;
Ar1 and Ar2 are each independently selected from the group consisting of substituted or unsubstituted C6-C60 aryl, substituted or unsubstituted C3-C60 heteroaryl;
the substituents in the substituted C6-C30 arylene, substituted C3-C30 heteroarylene, substituted C6-C60 aryl, substituted C6-C60 arylamine, substituted C3-C60 heteroarylamine, substituted C3-C60 heteroaryl, substituted C3-C30 heteroaryl, and substituted C3-C30 heteroarylene are selected from the group consisting of deuterium, halogen, cyano, C1-C12 alkyl, C3-C30 cycloalkyl, C6-C30 aryl, C3-C30 heteroaryl, C6-C60 arylamine, and C3-C60 heteroarylamine, or a combination of two thereof.

It can be understood that

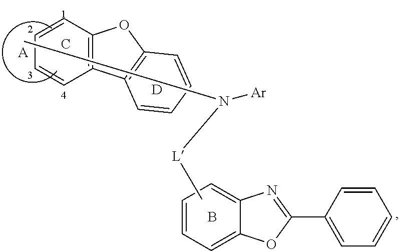

ring A in Formula (1) can be fused with ring C through positions 1, 2; 2, 3; 3, 4; N can be directly connected to any substitutable position in ring A, ring C and ring D; and L can be connected to any substitutable position in ring B.

Optionally, in the Formula (1), Ar, Ar1, Ar2 are selected from the group consisting of substituted or unsubstituted C6-C25 aryl, substituted or unsubstituted C6-C25 arylamine, substituted or unsubstituted C3-C25 heteroarylamine, substituted or unsubstituted C3-C20 heteroaryl;
wherein the substituents in the substituted C6-C25 aryl, substituted C6-C25 arylamine, substituted C3-C25 heteroarylamine, and substituted C3-C20 heteroaryl are each independently selected from the group consisting of deuterium, halogen, cyano, C1-C12 alkyl, C3-C12 cycloalkyl, C6-C30 aryl, C3-C30 heteroaryl, C6-C60 arylamine and C3-C60 heteroarylamine, or a combination of at least two thereof;
optionally, Ar is selected from substituted or unsubstituted B group, and the B group is selected from the group consisting of phenyl, naphthyl, biphenyl, phenanthryl, fluoranthenyl, chrysenyl, terphenyl, triphenylenyl, phenalenyl, phenylnaphthyl, naphthylphenyl, dimethylfluorenyl, phenylmethylfluorenyl, diphenylfluorenyl, pyridyl, pyridylphenyl, phenylpyridyl, spirobifluorenyl, benzodimethylfluorenyl, benzodiphenylfluorenyl, benzospirobifluorenyl, dibenzofuryl, benzonaphthofuryl, benzonaphthothienyl, spiro[fluorene-9,9′-xanthene]-yl, phenylmethylfluorenyl, dinaphthofuranyl, dinaphthothiophenyl, dibenzothiophenyl, and N, N-diphenylanilino;
wherein the substituents of the substituted B group are selected from the group consisting of deuterium, halogen, cyano, C1-C12 alkyl, C3-C12 cycloalkyl, C6-C30 aryl, C3-C30 heteroaryl, C6-C60 arylamine and C3-C60 heteroarylamine, or a combination of at least two thereof.

Optionally, Ar is selected from the group consisting of phenyl, naphthyl, biphenyl, chrysenyl, phenanthryl, terphenyl, phenylnaphthyl, naphthylphenyl, dimethylfluorenyl, diphenylfluorenyl, spirobifluorenyl, phenalenyl, dibenzofuryl, benzonaphthofuryl and N, N-diphenylanilino.

Optionally, L′ is selected from the group consisting of substituted or unsubstituted C6-C15 arylene; wherein the substituents in the substituted C6-C15 arylene are each independently selected from the group consisting of deuterium, halogen and C1-C62 alkyl, or a combination of at least two thereof;
optionally, L′ is selected from the group consisting of phenylene, biphenylene and naphthylene; and
optionally, L′ is selected from the group consisting of phenylene and naphthylene.

Optionally, the first compound has a structure shown in any one of N-1 to N-208 below:

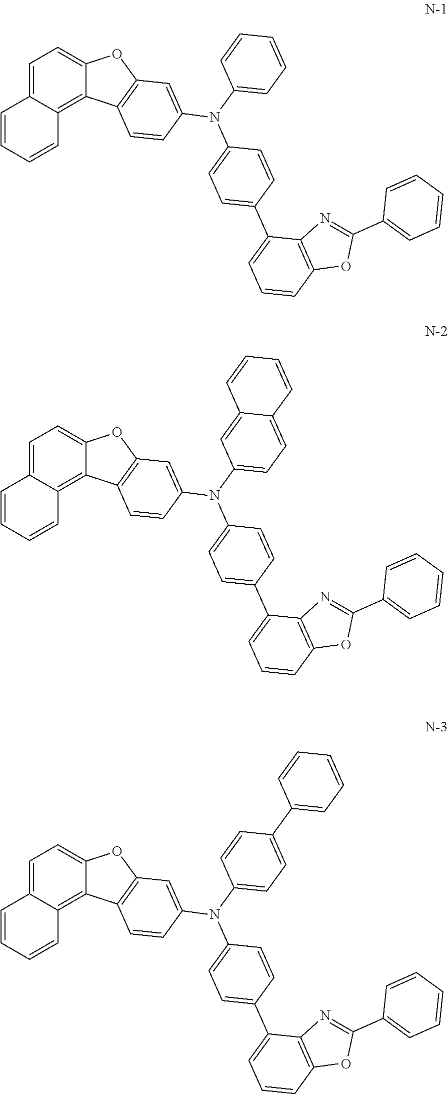 [SMILES: c1ccc(-c2nc3c(-c4ccc(N(c5ccccc5)c5ccc6c(c5)oc5ccc7ccccc7c56)cc4)cccc3o2)cc1 ; c1ccc(-c2nc3c(-c4ccc(N(c5ccc6ccccc6c5)c5ccc6c(c5)oc5ccc7ccccc7c56)cc4)cccc3o2)cc1 ; c1ccc(-c2ccc(N(c3ccc(-c4cccc5oc(-c6ccccc6)nc45)cc3)c3ccc4c(c3)oc3ccc5ccccc5c34)cc2)cc1]

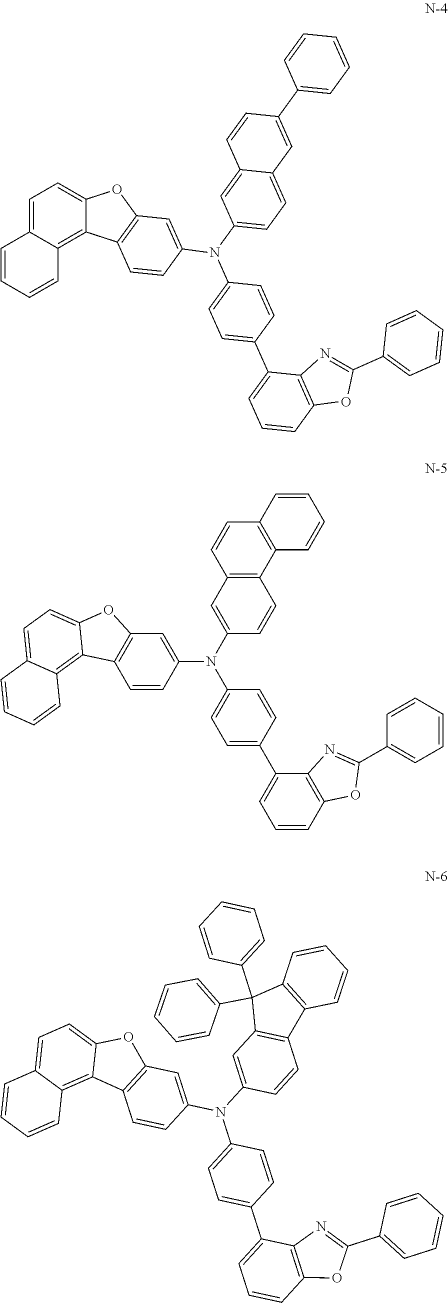 [SMILES: c1ccc(-c2ccc3cc(N(c4ccc(-c5cccc6oc(-c7ccccc7)nc56)cc4)c4ccc5c(c4)oc4ccc6ccccc6c45)ccc3c2)cc1 ; c1ccc(-c2nc3c(-c4ccc(N(c5ccc6c(ccc7ccccc76)c5)c5ccc6c(c5)oc5ccc7ccccc7c56)cc4)cccc3o2)cc1 ; c1ccc(-c2nc3c(-c4ccc(N(c5ccc6c(c5)C(c5ccccc5)(c5ccccc5)c5ccccc5-6)c5ccc6c(c5)oc5ccc7ccccc7c56)cc4)cccc3o2)cc1]

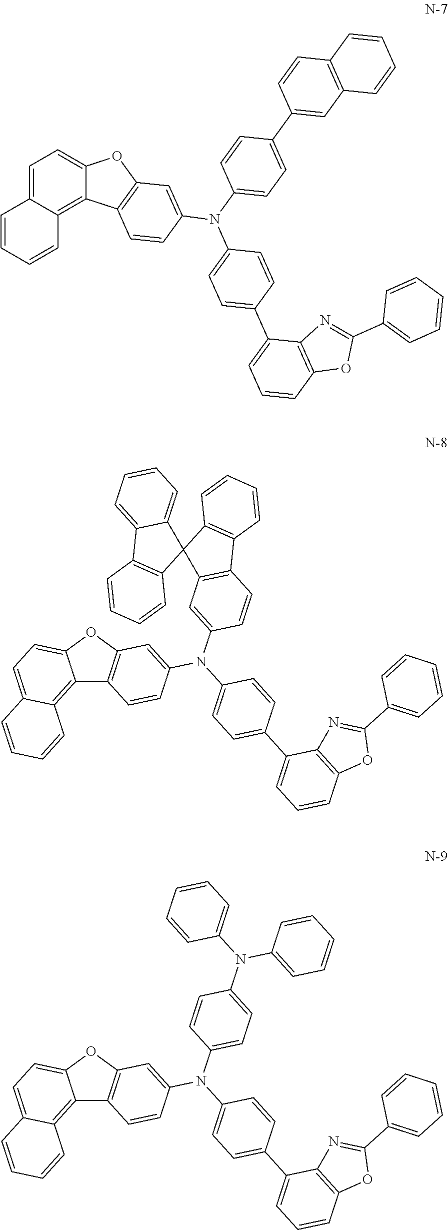 [SMILES: c1ccc(-c2nc3c(-c4ccc(N(c5ccc(-c6ccc7ccccc7c6)cc5)c5ccc6c(c5)oc5ccc7ccccc7c56)cc4)cccc3o2)cc1 ; c1ccc(-c2nc3c(-c4ccc(N(c5ccc6c(c5)C5(c7ccccc7-c7ccccc75)c5ccccc5-6)c5ccc6c(c5)oc5ccc7ccccc7c56)cc4)cccc3o2)cc1 ; c1ccc(-c2nc3c(-c4ccc(N(c5ccc(N(c6ccccc6)c6ccccc6)cc5)c5ccc6c(c5)oc5ccc7ccccc7c56)cc4)cccc3o2)cc1]

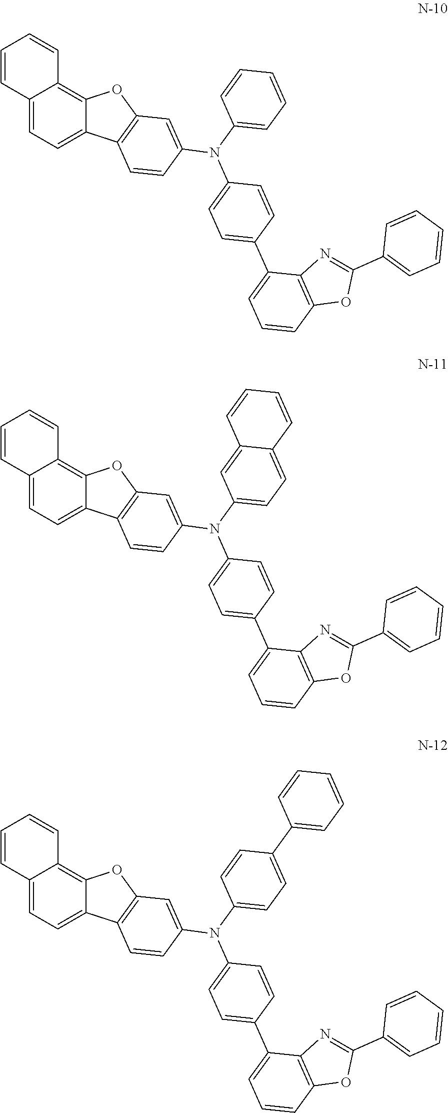 [SMILES: c1ccc(-c2nc3c(-c4ccc(N(c5ccccc5)c5ccc6c(c5)oc5c7ccccc7ccc65)cc4)cccc3o2)cc1 ; c1ccc(-c2nc3c(-c4ccc(N(c5ccc6ccccc6c5)c5ccc6c(c5)oc5c7ccccc7ccc65)cc4)cccc3o2)cc1 ; c1ccc(-c2ccc(N(c3ccc(-c4cccc5oc(-c6ccccc6)nc45)cc3)c3ccc4c(c3)oc3c5ccccc5ccc43)cc2)cc1]

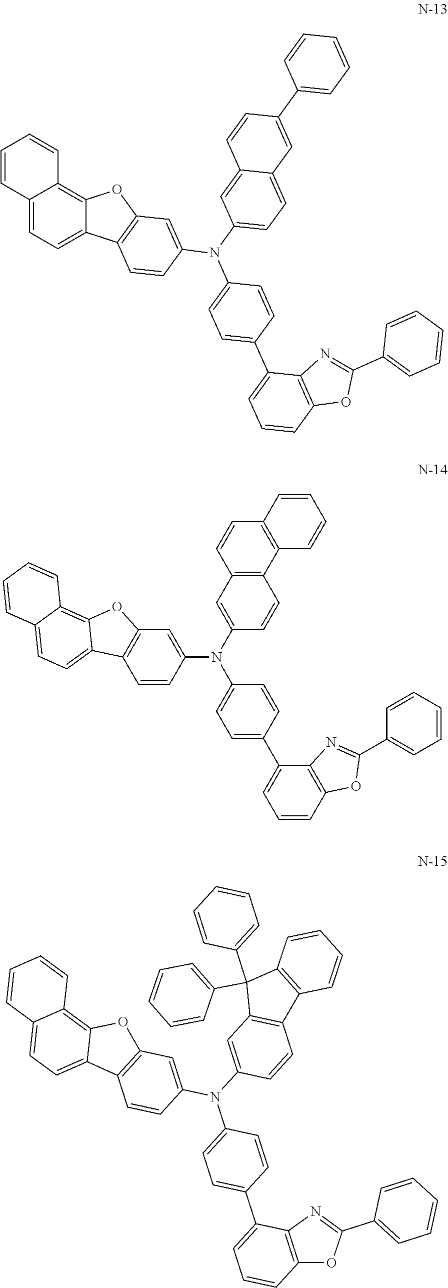 [SMILES: c1ccc(-c2ccc3cc(N(c4ccc(-c5cccc6oc(-c7ccccc7)nc56)cc4)c4ccc5c(c4)oc4c6ccccc6ccc54)ccc3c2)cc1 ; c1ccc(-c2nc3c(-c4ccc(N(c5ccc6c(ccc7ccccc76)c5)c5ccc6c(c5)oc5c7ccccc7ccc65)cc4)cccc3o2)cc1 ; c1ccc(-c2nc3c(-c4ccc(N(c5ccc6c(c5)C(c5ccccc5)(c5ccccc5)c5ccccc5-6)c5ccc6c(c5)oc5c7ccccc7ccc65)cc4)cccc3o2)cc1]

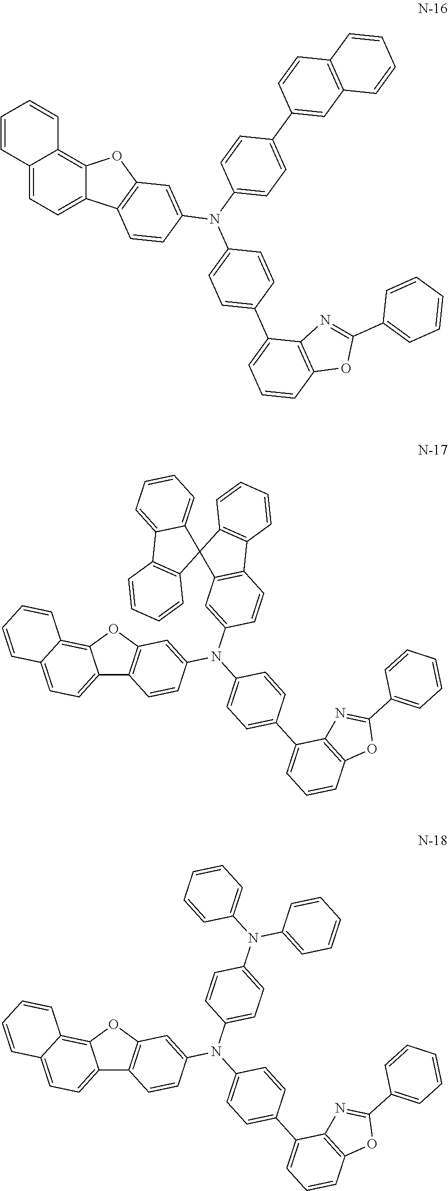 [SMILES: c1ccc(-c2nc3c(-c4ccc(N(c5ccc(-c6ccc7ccccc7c6)cc5)c5ccc6c(c5)oc5c7ccccc7ccc65)cc4)cccc3o2)cc1 ; c1ccc(-c2nc3c(-c4ccc(N(c5ccc6c(c5)C5(c7ccccc7-c7ccccc75)c5ccccc5-6)c5ccc6c(c5)oc5c7ccccc7ccc65)cc4)cccc3o2)cc1 ; c1ccc(-c2nc3c(-c4ccc(N(c5ccc(N(c6ccccc6)c6ccccc6)cc5)c5ccc6c(c5)oc5c7ccccc7ccc65)cc4)cccc3o2)cc1]

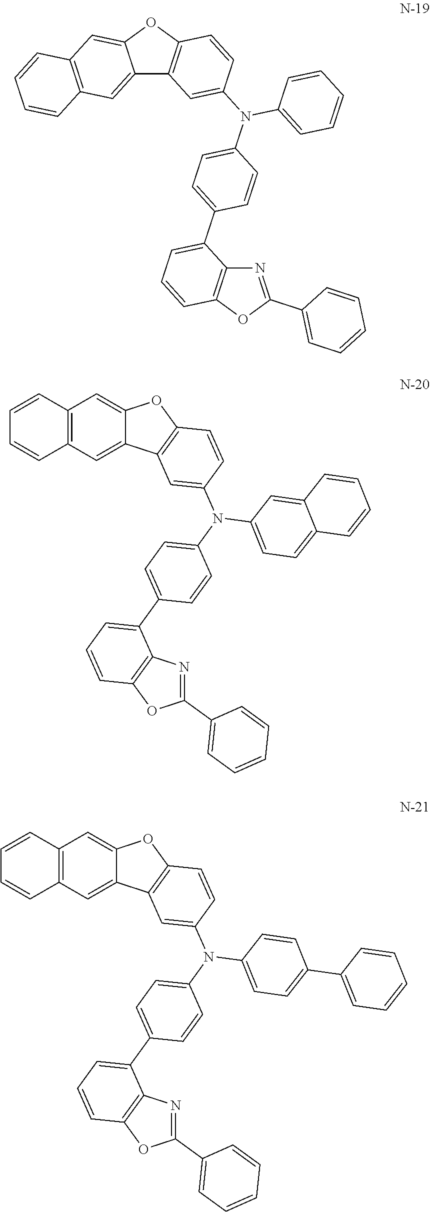 [SMILES: c1ccc(-c2nc3c(-c4ccc(N(c5ccccc5)c5ccc6oc7cc8ccccc8cc7c6c5)cc4)cccc3o2)cc1 ; c1ccc(-c2nc3c(-c4ccc(N(c5ccc6ccccc6c5)c5ccc6oc7cc8ccccc8cc7c6c5)cc4)cccc3o2)cc1 ; c1ccc(-c2ccc(N(c3ccc(-c4cccc5oc(-c6ccccc6)nc45)cc3)c3ccc4oc5cc6ccccc6cc5c4c3)cc2)cc1]

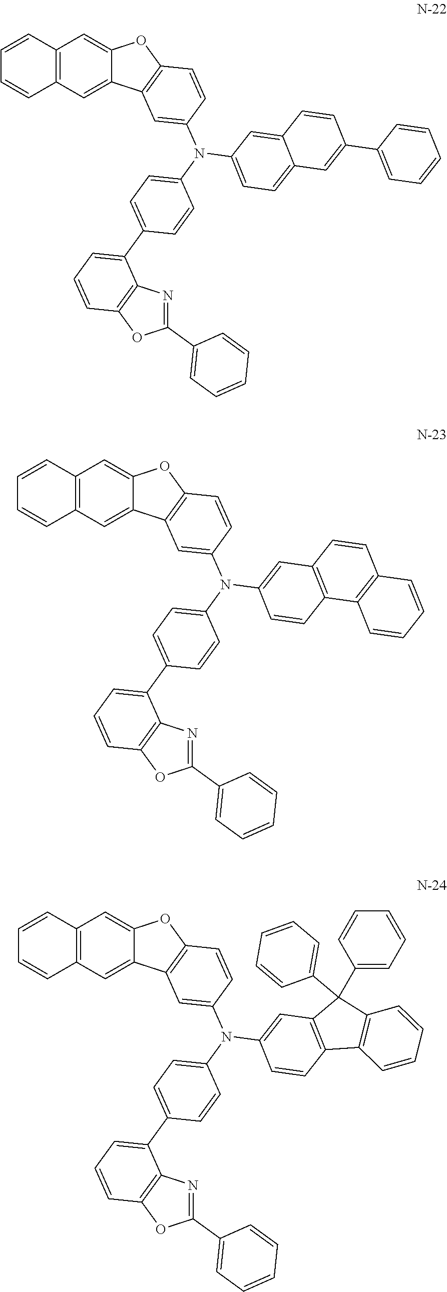 [SMILES: c1ccc(-c2ccc3cc(N(c4ccc(-c5cccc6oc(-c7ccccc7)nc56)cc4)c4ccc5oc6cc7ccccc7cc6c5c4)ccc3c2)cc1 ; c1ccc(-c2nc3c(-c4ccc(N(c5ccc6c(ccc7ccccc76)c5)c5ccc6oc7cc8ccccc8cc7c6c5)cc4)cccc3o2)cc1 ; c1ccc(-c2nc3c(-c4ccc(N(c5ccc6c(c5)C(c5ccccc5)(c5ccccc5)c5ccccc5-6)c5ccc6oc7cc8ccccc8cc7c6c5)cc4)cccc3o2)cc1]

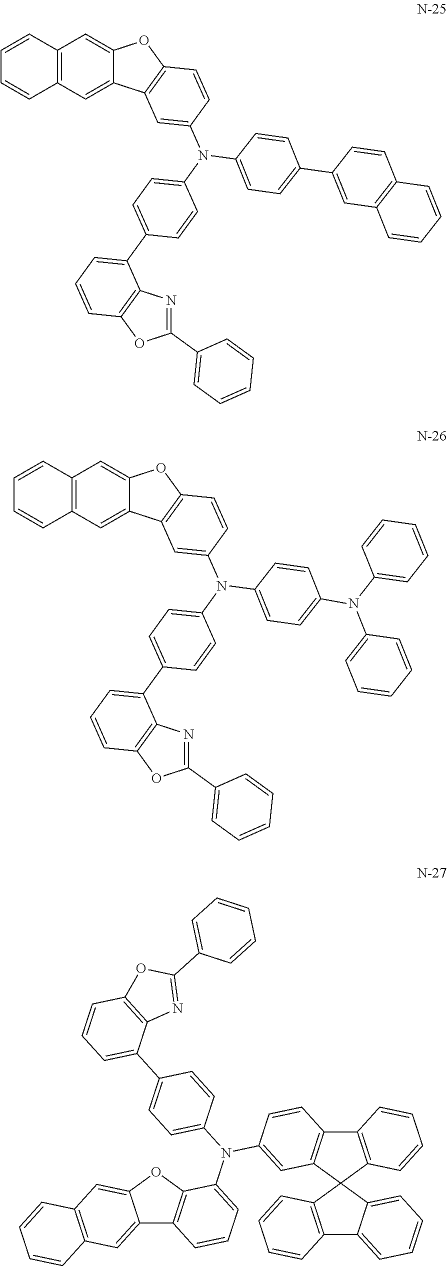 [SMILES: c1ccc(-c2nc3c(-c4ccc(N(c5ccc(-c6ccc7ccccc7c6)cc5)c5ccc6oc7cc8ccccc8cc7c6c5)cc4)cccc3o2)cc1 ; c1ccc(-c2nc3c(-c4ccc(N(c5ccc(N(c6ccccc6)c6ccccc6)cc5)c5ccc6oc7cc8ccccc8cc7c6c5)cc4)cccc3o2)cc1 ; c1ccc(-c2nc3c(-c4ccc(N(c5ccc6c(c5)C5(c7ccccc7-c7ccccc75)c5ccccc5-6)c5cccc6c5oc5cc7ccccc7cc56)cc4)cccc3o2)cc1]

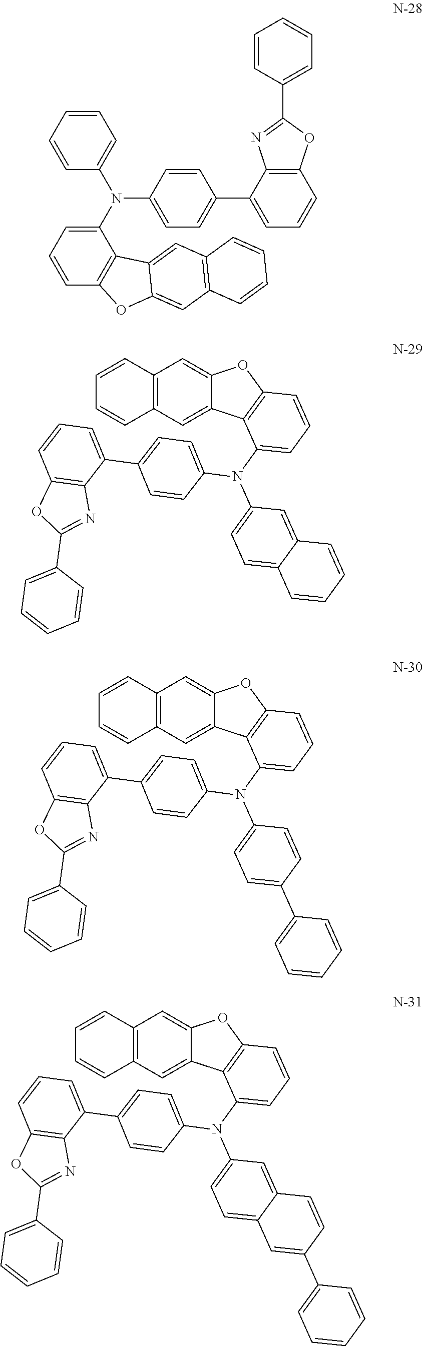 [SMILES: c1ccc(-c2nc3c(-c4ccc(N(c5ccccc5)c5cccc6oc7cc8ccccc8cc7c56)cc4)cccc3o2)cc1 ; c1ccc(-c2nc3c(-c4ccc(N(c5ccc6ccccc6c5)c5cccc6oc7cc8ccccc8cc7c56)cc4)cccc3o2)cc1 ; c1ccc(-c2ccc(N(c3ccc(-c4cccc5oc(-c6ccccc6)nc45)cc3)c3cccc4oc5cc6ccccc6cc5c34)cc2)cc1 ; c1ccc(-c2ccc3cc(N(c4ccc(-c5cccc6oc(-c7ccccc7)nc56)cc4)c4cccc5oc6cc7ccccc7cc6c45)ccc3c2)cc1]

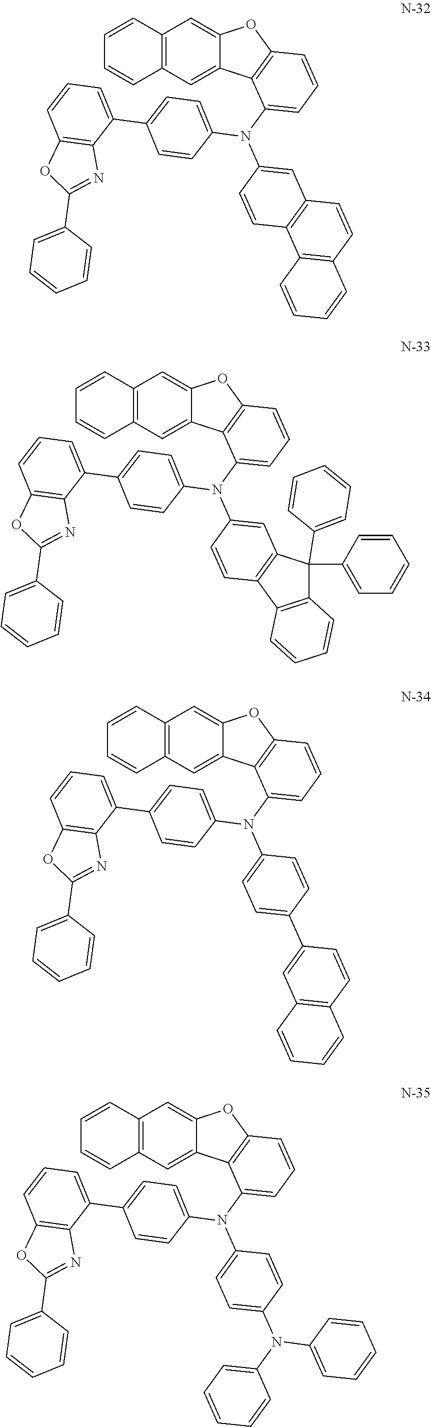 [SMILES: c1ccc(-c2nc3c(-c4ccc(N(c5ccc6c(ccc7ccccc76)c5)c5cccc6oc7cc8ccccc8cc7c56)cc4)cccc3o2)cc1 ; c1ccc(-c2nc3c(-c4ccc(N(c5ccc6c(c5)C(c5ccccc5)(c5ccccc5)c5ccccc5-6)c5cccc6oc7cc8ccccc8cc7c56)cc4)cccc3o2)cc1 ; c1ccc(-c2nc3c(-c4ccc(N(c5ccc(-c6ccc7ccccc7c6)cc5)c5cccc6oc7cc8ccccc8cc7c56)cc4)cccc3o2)cc1 ; c1ccc(-c2nc3c(-c4ccc(N(c5ccc(N(c6ccccc6)c6ccccc6)cc5)c5cccc6oc7cc8ccccc8cc7c56)cc4)cccc3o2)cc1]

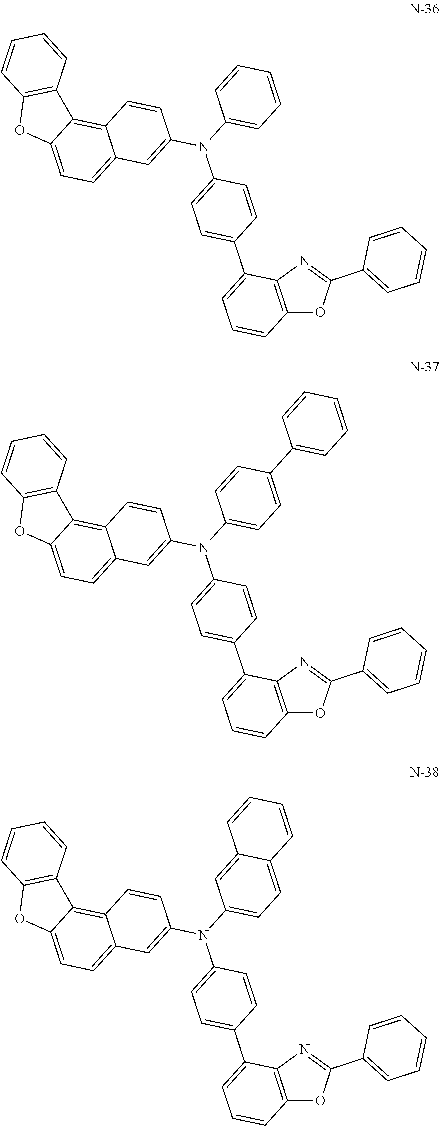 [SMILES: c1ccc(-c2nc3c(-c4ccc(N(c5ccccc5)c5ccc6c(ccc7oc8ccccc8c76)c5)cc4)cccc3o2)cc1 ; c1ccc(-c2ccc(N(c3ccc(-c4cccc5oc(-c6ccccc6)nc45)cc3)c3ccc4c(ccc5oc6ccccc6c54)c3)cc2)cc1 ; c1ccc(-c2nc3c(-c4ccc(N(c5ccc6ccccc6c5)c5ccc6c(ccc7oc8ccccc8c76)c5)cc4)cccc3o2)cc1]

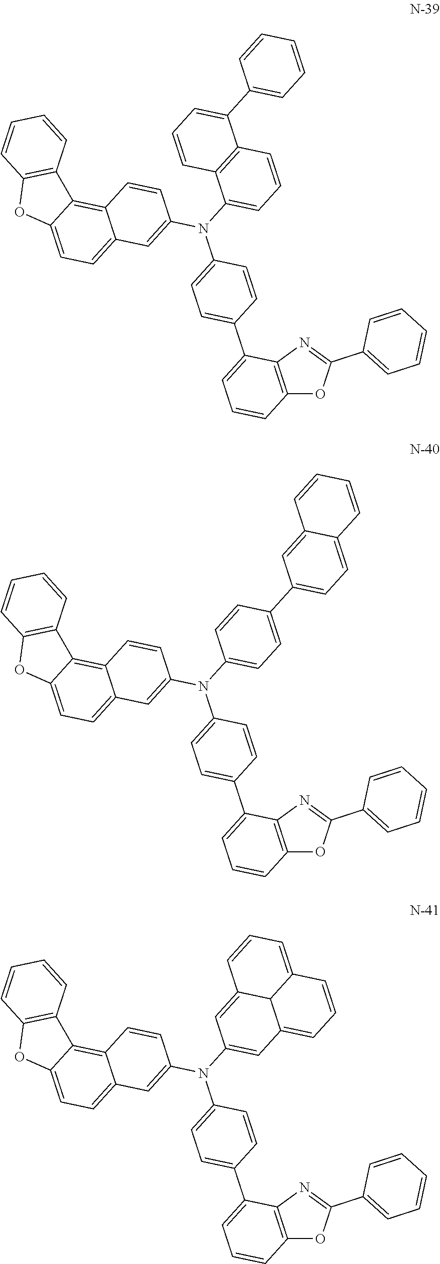 [SMILES: c1ccc(-c2nc3c(-c4ccc(N(c5ccc6c(ccc7oc8ccccc8c76)c5)c5cccc6c(-c7ccccc7)cccc56)cc4)cccc3o2)cc1 ; c1ccc(-c2nc3c(-c4ccc(N(c5ccc(-c6ccc7ccccc7c6)cc5)c5ccc6c(ccc7oc8ccccc8c76)c5)cc4)cccc3o2)cc1 ; C1=CC2=CC=CC3=CC(N(c4ccc(-c5cccc6oc(-c7ccccc7)nc56)cc4)c4ccc5c(ccc6oc7ccccc7c65)c4)=CC(=C1)C23]

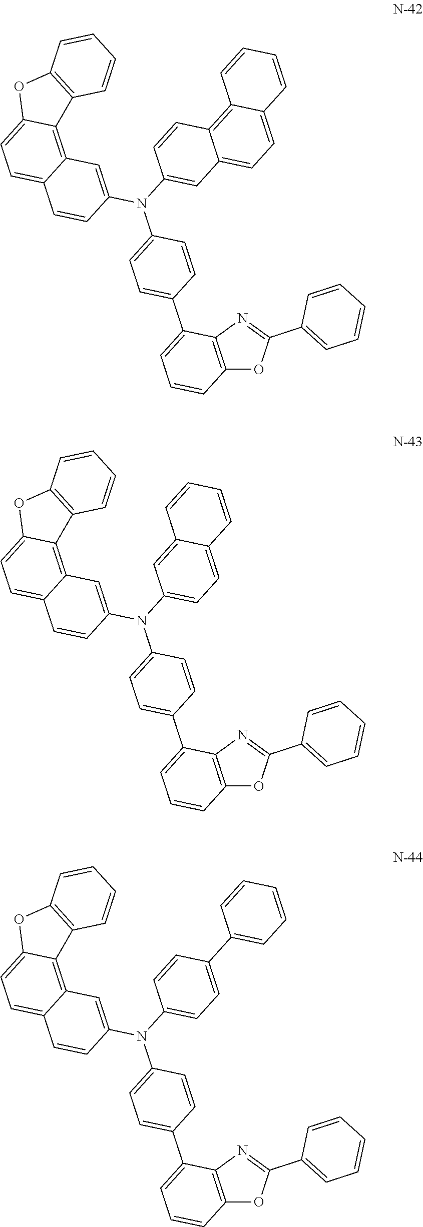 [SMILES: c1ccc(-c2nc3c(-c4ccc(N(c5ccc6c(ccc7ccccc76)c5)c5ccc6ccc7oc8ccccc8c7c6c5)cc4)cccc3o2)cc1 ; c1ccc(-c2nc3c(-c4ccc(N(c5ccc6ccccc6c5)c5ccc6ccc7oc8ccccc8c7c6c5)cc4)cccc3o2)cc1 ; c1ccc(-c2ccc(N(c3ccc(-c4cccc5oc(-c6ccccc6)nc45)cc3)c3ccc4ccc5oc6ccccc6c5c4c3)cc2)cc1]

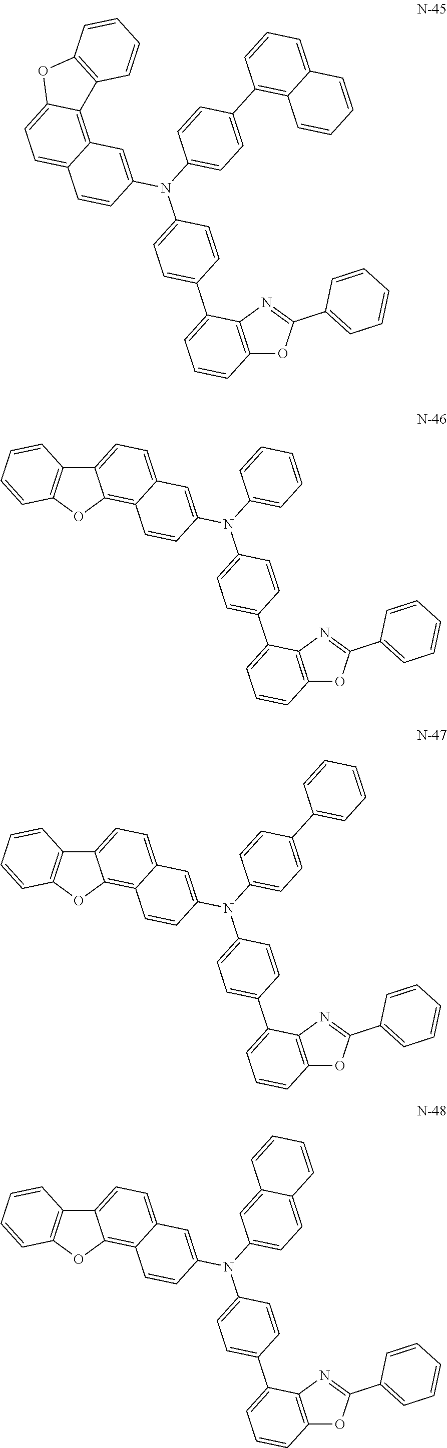 [SMILES: c1ccc(-c2nc3c(-c4ccc(N(c5ccc(-c6cccc7ccccc67)cc5)c5ccc6ccc7oc8ccccc8c7c6c5)cc4)cccc3o2)cc1 ; c1ccc(-c2nc3c(-c4ccc(N(c5ccccc5)c5ccc6c(ccc7c8ccccc8oc67)c5)cc4)cccc3o2)cc1 ; c1ccc(-c2ccc(N(c3ccc(-c4cccc5oc(-c6ccccc6)nc45)cc3)c3ccc4c(ccc5c6ccccc6oc45)c3)cc2)cc1 ; c1ccc(-c2nc3c(-c4ccc(N(c5ccc6ccccc6c5)c5ccc6c(ccc7c8ccccc8oc67)c5)cc4)cccc3o2)cc1]

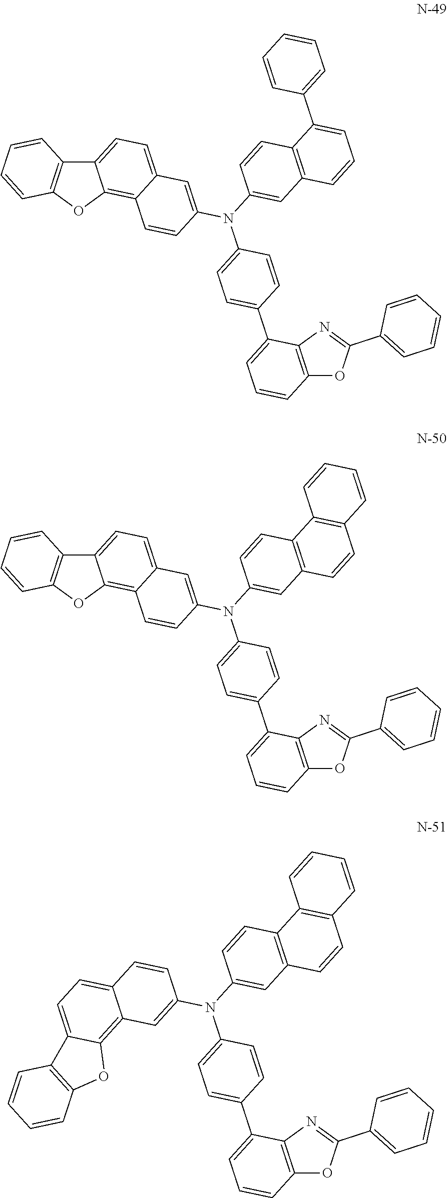 [SMILES: c1ccc(-c2nc3c(-c4ccc(N(c5ccc6c(-c7ccccc7)cccc6c5)c5ccc6c(ccc7c8ccccc8oc67)c5)cc4)cccc3o2)cc1 ; c1ccc(-c2nc3c(-c4ccc(N(c5ccc6c(ccc7ccccc76)c5)c5ccc6c(ccc7c8ccccc8oc67)c5)cc4)cccc3o2)cc1 ; c1ccc(-c2nc3c(-c4ccc(N(c5ccc6c(ccc7ccccc76)c5)c5ccc6ccc7c8ccccc8oc7c6c5)cc4)cccc3o2)cc1]

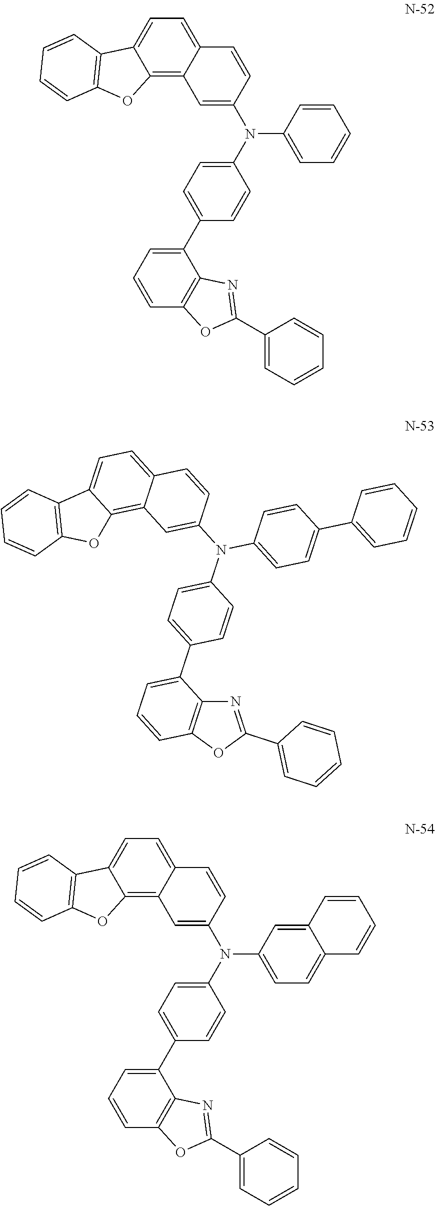 [SMILES: c1ccc(-c2nc3c(-c4ccc(N(c5ccccc5)c5ccc6ccc7c8ccccc8oc7c6c5)cc4)cccc3o2)cc1 ; c1ccc(-c2ccc(N(c3ccc(-c4cccc5oc(-c6ccccc6)nc45)cc3)c3ccc4ccc5c6ccccc6oc5c4c3)cc2)cc1 ; c1ccc(-c2nc3c(-c4ccc(N(c5ccc6ccccc6c5)c5ccc6ccc7c8ccccc8oc7c6c5)cc4)cccc3o2)cc1]

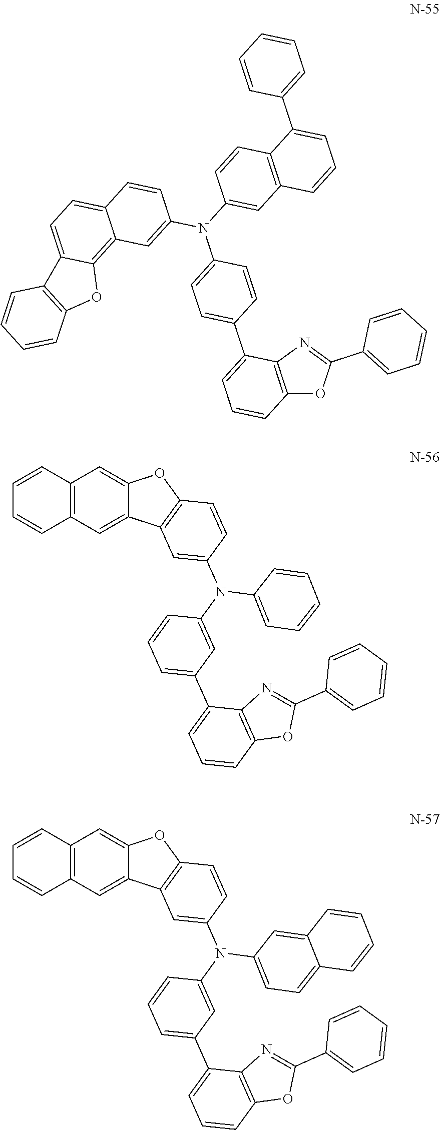 [SMILES: c1ccc(-c2nc3c(-c4ccc(N(c5ccc6c(-c7ccccc7)cccc6c5)c5ccc6ccc7c8ccccc8oc7c6c5)cc4)cccc3o2)cc1 ; c1ccc(-c2nc3c(-c4cccc(N(c5ccccc5)c5ccc6oc7cc8ccccc8cc7c6c5)c4)cccc3o2)cc1 ; c1ccc(-c2nc3c(-c4cccc(N(c5ccc6ccccc6c5)c5ccc6oc7cc8ccccc8cc7c6c5)c4)cccc3o2)cc1]

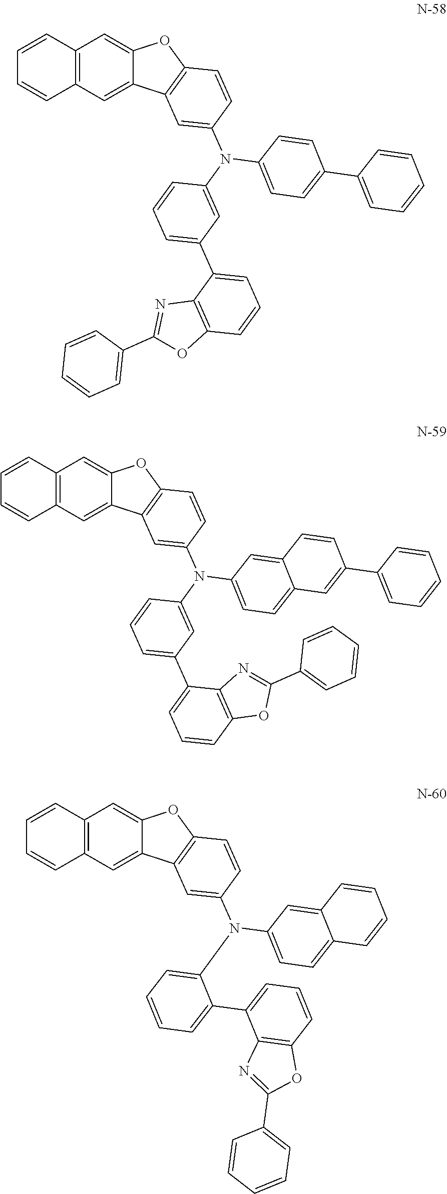 [SMILES: c1ccc(-c2ccc(N(c3cccc(-c4cccc5oc(-c6ccccc6)nc45)c3)c3ccc4oc5cc6ccccc6cc5c4c3)cc2)cc1 ; c1ccc(-c2ccc3cc(N(c4cccc(-c5cccc6oc(-c7ccccc7)nc56)c4)c4ccc5oc6cc7ccccc7cc6c5c4)ccc3c2)cc1 ; c1ccc(-c2nc3c(-c4ccccc4N(c4ccc5ccccc5c4)c4ccc5oc6cc7ccccc7cc6c5c4)cccc3o2)cc1]

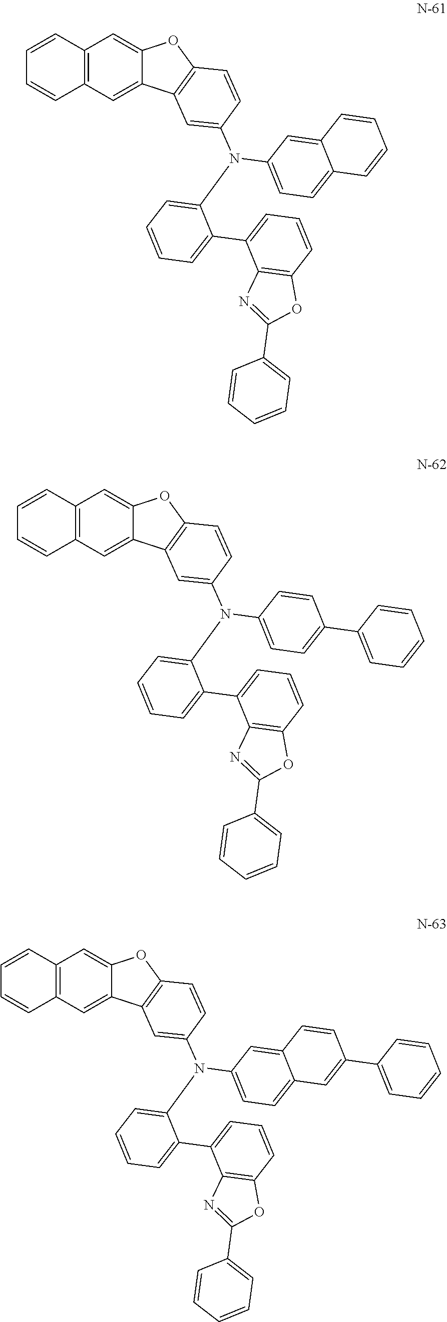 [SMILES: c1ccc(-c2nc3c(-c4ccccc4N(c4ccc5ccccc5c4)c4ccc5oc6cc7ccccc7cc6c5c4)cccc3o2)cc1 ; c1ccc(-c2ccc(N(c3ccc4oc5cc6ccccc6cc5c4c3)c3ccccc3-c3cccc4oc(-c5ccccc5)nc34)cc2)cc1 ; c1ccc(-c2ccc3cc(N(c4ccc5oc6cc7ccccc7cc6c5c4)c4ccccc4-c4cccc5oc(-c6ccccc6)nc45)ccc3c2)cc1]

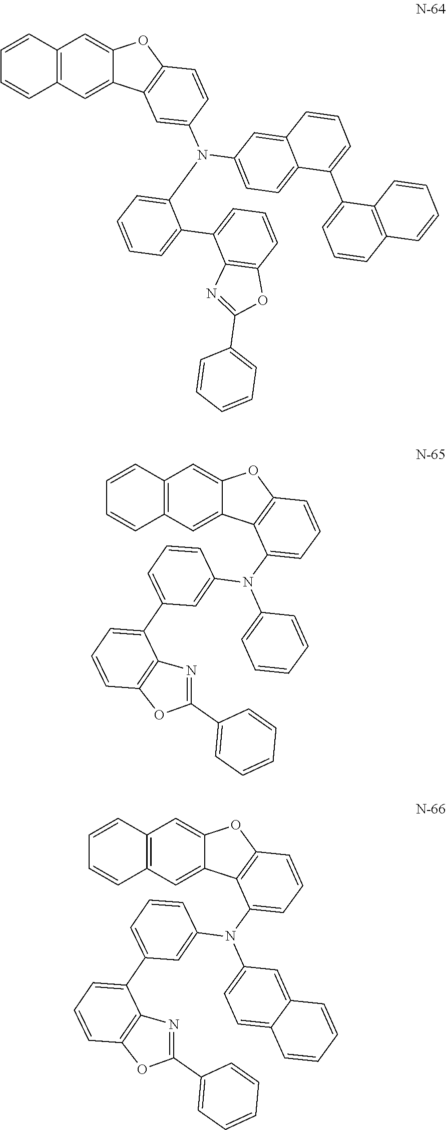 [SMILES: c1ccc(-c2nc3c(-c4ccccc4N(c4ccc5c(-c6cccc7ccccc67)cccc5c4)c4ccc5oc6cc7ccccc7cc6c5c4)cccc3o2)cc1 ; c1ccc(-c2nc3c(-c4cccc(N(c5ccccc5)c5cccc6oc7cc8ccccc8cc7c56)c4)cccc3o2)cc1 ; c1ccc(-c2nc3c(-c4cccc(N(c5ccc6ccccc6c5)c5cccc6oc7cc8ccccc8cc7c56)c4)cccc3o2)cc1]

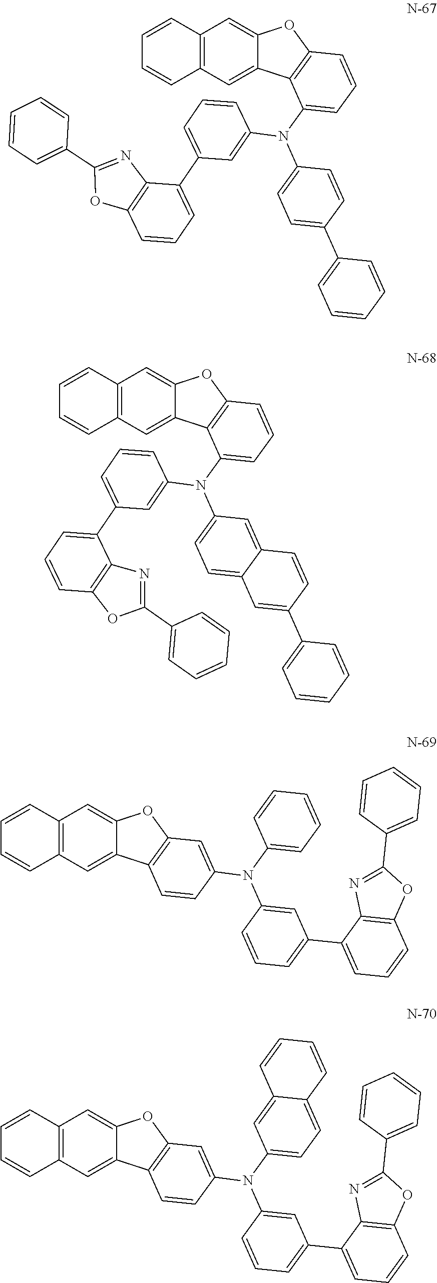 [SMILES: c1ccc(-c2ccc(N(c3cccc(-c4cccc5oc(-c6ccccc6)nc45)c3)c3cccc4oc5cc6ccccc6cc5c34)cc2)cc1 ; c1ccc(-c2ccc3cc(N(c4cccc(-c5cccc6oc(-c7ccccc7)nc56)c4)c4cccc5oc6cc7ccccc7cc6c45)ccc3c2)cc1 ; c1ccc(-c2nc3c(-c4cccc(N(c5ccccc5)c5ccc6c(c5)oc5cc7ccccc7cc56)c4)cccc3o2)cc1 ; c1ccc(-c2nc3c(-c4cccc(N(c5ccc6ccccc6c5)c5ccc6c(c5)oc5cc7ccccc7cc56)c4)cccc3o2)cc1]

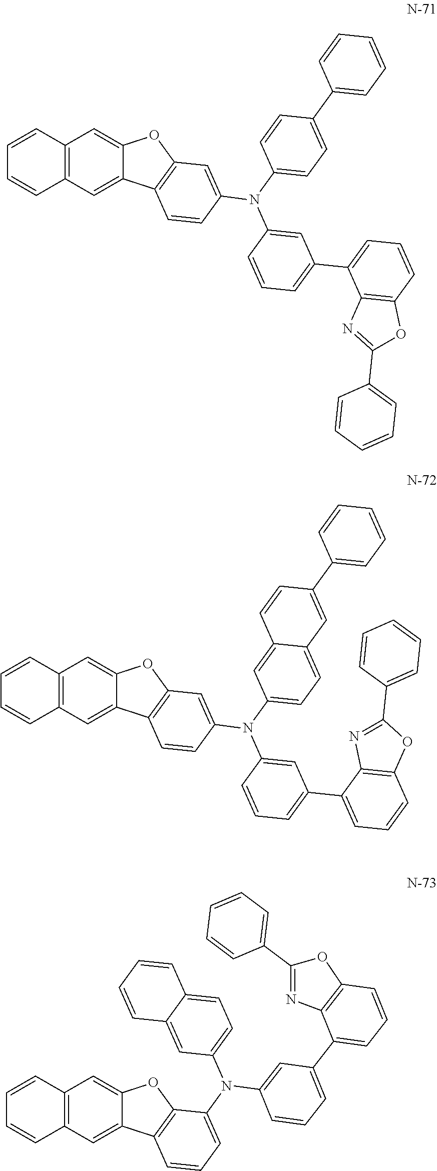 [SMILES: c1ccc(-c2ccc(N(c3cccc(-c4cccc5oc(-c6ccccc6)nc45)c3)c3ccc4c(c3)oc3cc5ccccc5cc34)cc2)cc1 ; c1ccc(-c2ccc3cc(N(c4cccc(-c5cccc6oc(-c7ccccc7)nc56)c4)c4ccc5c(c4)oc4cc6ccccc6cc45)ccc3c2)cc1 ; c1ccc(-c2nc3c(-c4cccc(N(c5ccc6ccccc6c5)c5cccc6c5oc5cc7ccccc7cc56)c4)cccc3o2)cc1]

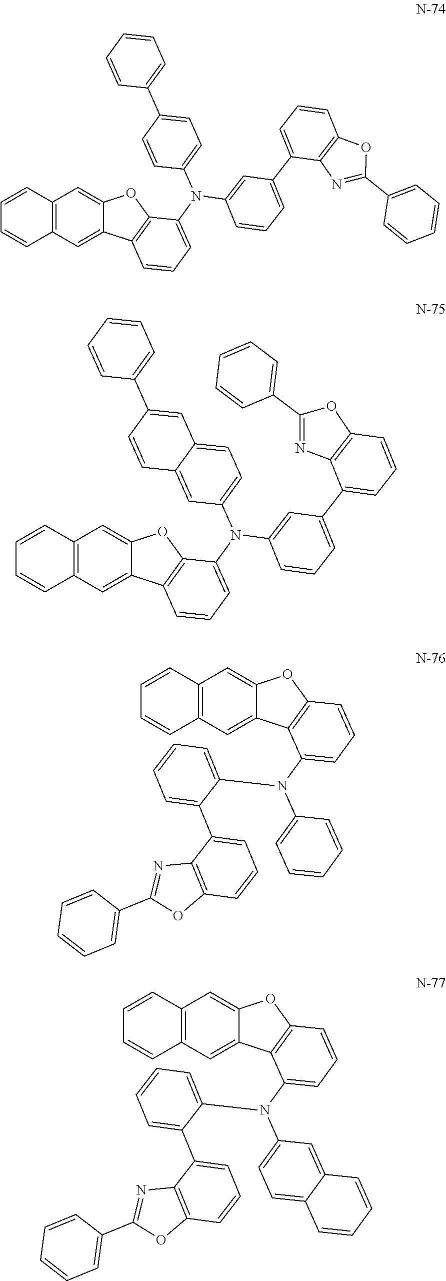 [SMILES: c1ccc(-c2ccc(N(c3cccc(-c4cccc5oc(-c6ccccc6)nc45)c3)c3cccc4c3oc3cc5ccccc5cc34)cc2)cc1 ; c1ccc(-c2ccc3cc(N(c4cccc(-c5cccc6oc(-c7ccccc7)nc56)c4)c4cccc5c4oc4cc6ccccc6cc45)ccc3c2)cc1 ; c1ccc(-c2nc3c(-c4ccccc4N(c4ccccc4)c4cccc5oc6cc7ccccc7cc6c45)cccc3o2)cc1 ; c1ccc(-c2nc3c(-c4ccccc4N(c4ccc5ccccc5c4)c4cccc5oc6cc7ccccc7cc6c45)cccc3o2)cc1]

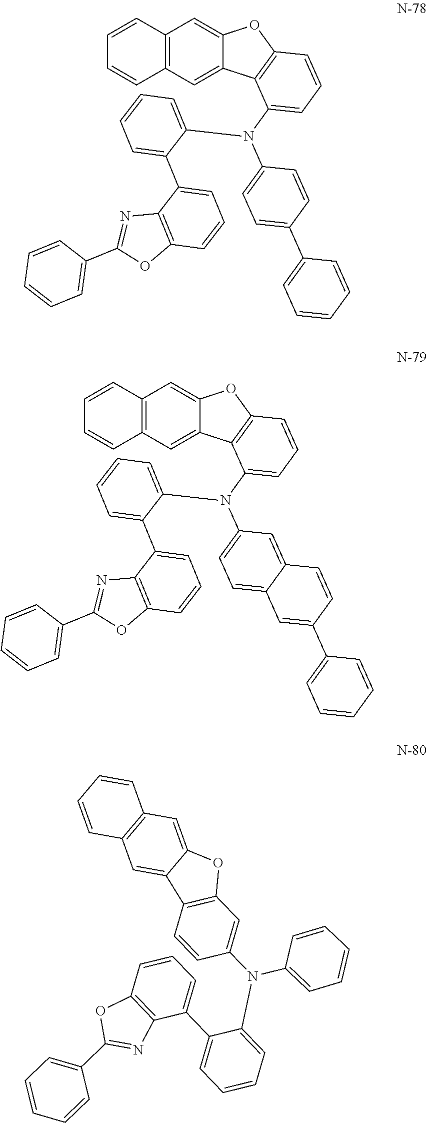 [SMILES: c1ccc(-c2ccc(N(c3ccccc3-c3cccc4oc(-c5ccccc5)nc34)c3cccc4oc5cc6ccccc6cc5c34)cc2)cc1 ; c1ccc(-c2ccc3cc(N(c4ccccc4-c4cccc5oc(-c6ccccc6)nc45)c4cccc5oc6cc7ccccc7cc6c45)ccc3c2)cc1 ; c1ccc(-c2nc3c(-c4ccccc4N(c4ccccc4)c4ccc5c(c4)oc4cc6ccccc6cc45)cccc3o2)cc1]

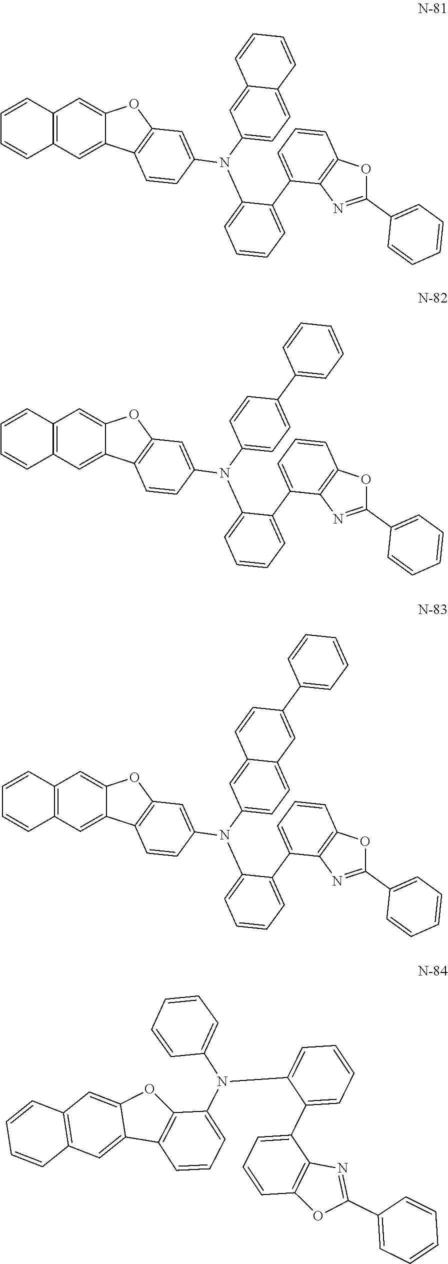 [SMILES: c1ccc(-c2nc3c(-c4ccccc4N(c4ccc5ccccc5c4)c4ccc5c(c4)oc4cc6ccccc6cc45)cccc3o2)cc1 ; c1ccc(-c2ccc(N(c3ccc4c(c3)oc3cc5ccccc5cc34)c3ccccc3-c3cccc4oc(-c5ccccc5)nc34)cc2)cc1 ; c1ccc(-c2ccc3cc(N(c4ccc5c(c4)oc4cc6ccccc6cc45)c4ccccc4-c4cccc5oc(-c6ccccc6)nc45)ccc3c2)cc1 ; c1ccc(-c2nc3c(-c4ccccc4N(c4ccccc4)c4cccc5c4oc4cc6ccccc6cc45)cccc3o2)cc1]

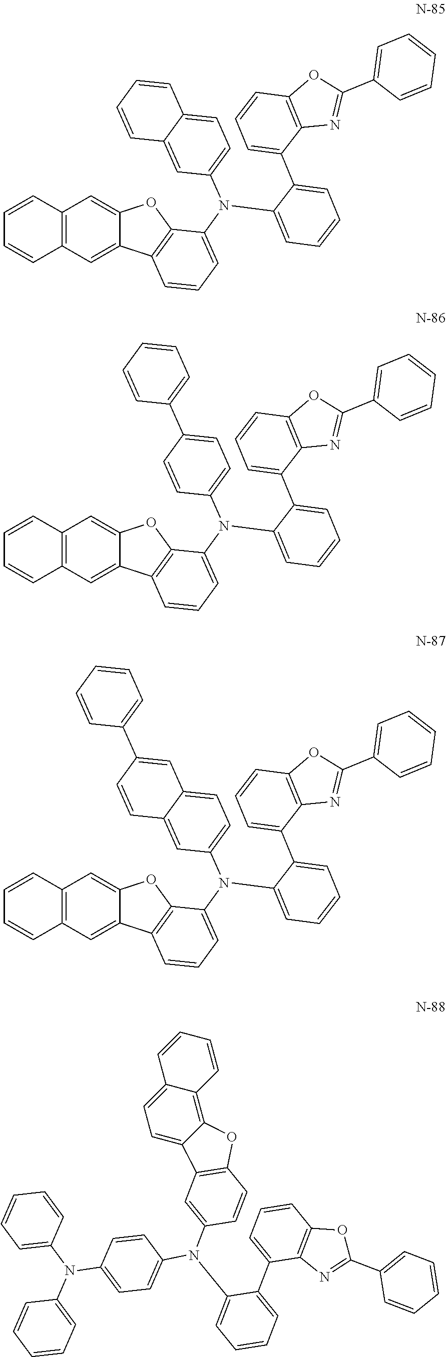 [SMILES: c1ccc(-c2nc3c(-c4ccccc4N(c4ccc5ccccc5c4)c4cccc5c4oc4cc6ccccc6cc45)cccc3o2)cc1 ; c1ccc(-c2ccc(N(c3ccccc3-c3cccc4oc(-c5ccccc5)nc34)c3cccc4c3oc3cc5ccccc5cc34)cc2)cc1 ; c1ccc(-c2ccc3cc(N(c4ccccc4-c4cccc5oc(-c6ccccc6)nc45)c4cccc5c4oc4cc6ccccc6cc45)ccc3c2)cc1 ; c1ccc(-c2nc3c(-c4ccccc4N(c4ccc(N(c5ccccc5)c5ccccc5)cc4)c4ccc5oc6c7ccccc7ccc6c5c4)cccc3o2)cc1]

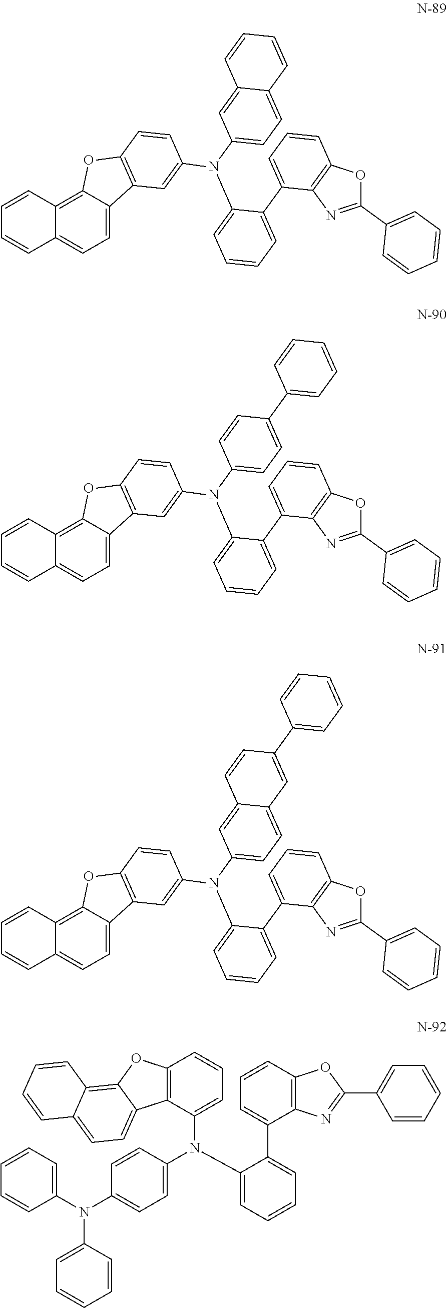 [SMILES: c1ccc(-c2nc3c(-c4ccccc4N(c4ccc5ccccc5c4)c4ccc5oc6c7ccccc7ccc6c5c4)cccc3o2)cc1 ; c1ccc(-c2ccc(N(c3ccc4oc5c6ccccc6ccc5c4c3)c3ccccc3-c3cccc4oc(-c5ccccc5)nc34)cc2)cc1 ; c1ccc(-c2ccc3cc(N(c4ccc5oc6c7ccccc7ccc6c5c4)c4ccccc4-c4cccc5oc(-c6ccccc6)nc45)ccc3c2)cc1 ; c1ccc(-c2nc3c(-c4ccccc4N(c4ccc(N(c5ccccc5)c5ccccc5)cc4)c4cccc5oc6c7ccccc7ccc6c45)cccc3o2)cc1]

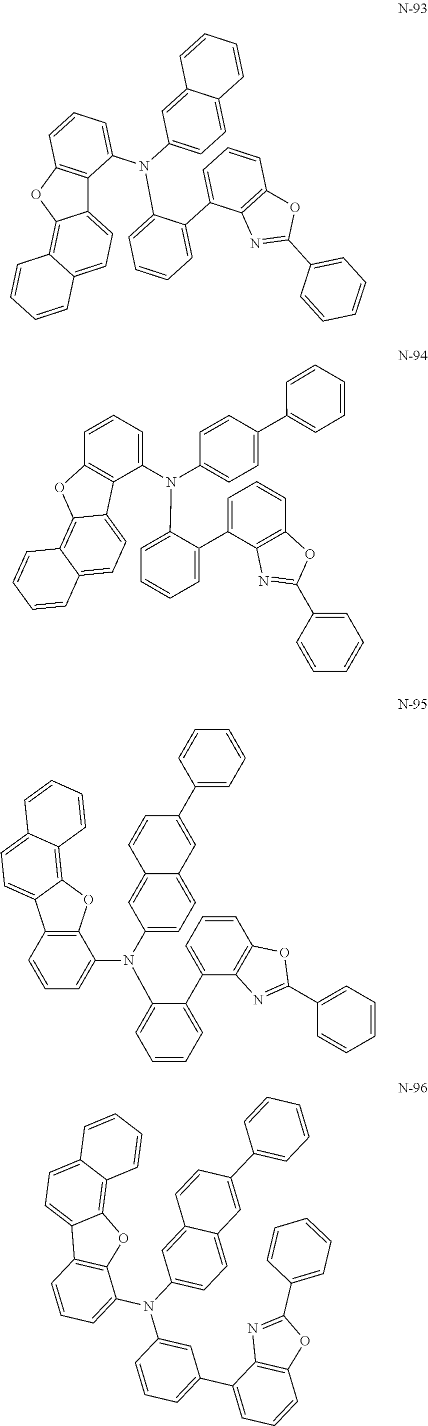

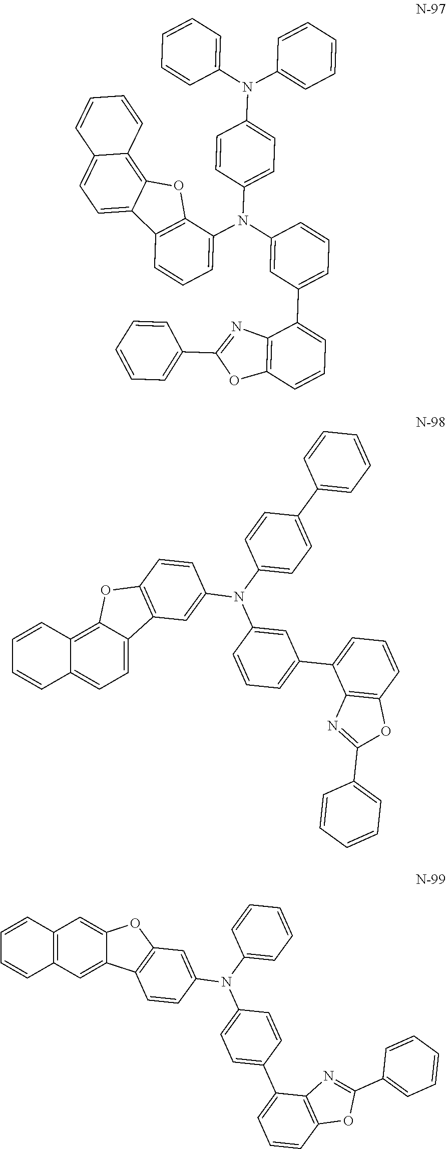 [SMILES: c1ccc(-c2nc3c(-c4cccc(N(c5ccc(N(c6ccccc6)c6ccccc6)cc5)c5cccc6c5oc5c7ccccc7ccc65)c4)cccc3o2)cc1 ; c1ccc(-c2ccc(N(c3cccc(-c4cccc5oc(-c6ccccc6)nc45)c3)c3ccc4oc5c6ccccc6ccc5c4c3)cc2)cc1 ; c1ccc(-c2nc3c(-c4ccc(N(c5ccccc5)c5ccc6c(c5)oc5cc7ccccc7cc56)cc4)cccc3o2)cc1]

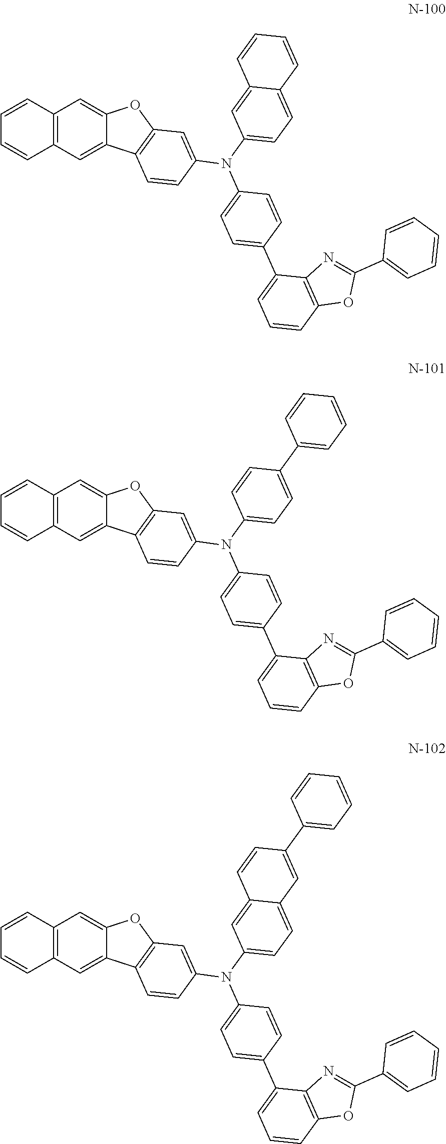 [SMILES: c1ccc(-c2nc3c(-c4ccc(N(c5ccc6ccccc6c5)c5ccc6c(c5)oc5cc7ccccc7cc56)cc4)cccc3o2)cc1 ; c1ccc(-c2ccc(N(c3ccc(-c4cccc5oc(-c6ccccc6)nc45)cc3)c3ccc4c(c3)oc3cc5ccccc5cc34)cc2)cc1 ; c1ccc(-c2ccc3cc(N(c4ccc(-c5cccc6oc(-c7ccccc7)nc56)cc4)c4ccc5c(c4)oc4cc6ccccc6cc45)ccc3c2)cc1]

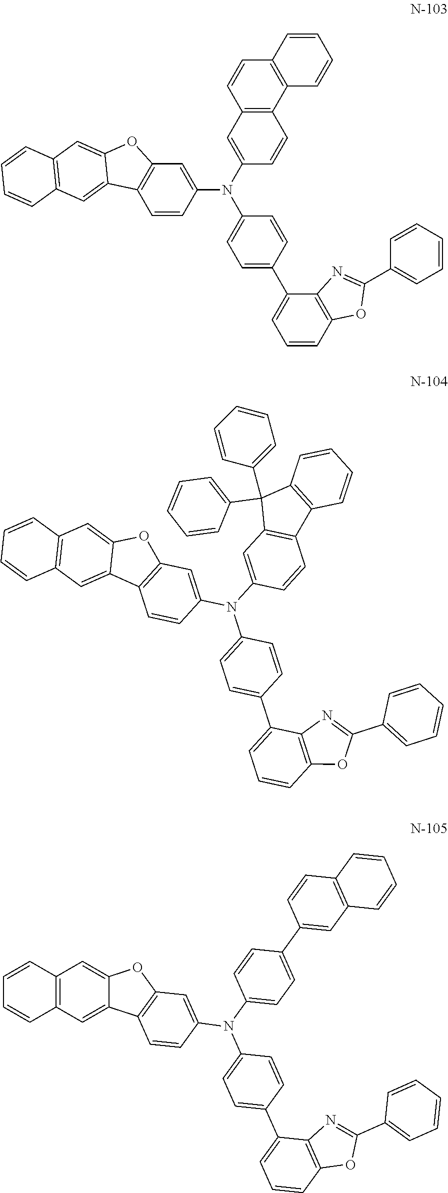 [SMILES: c1ccc(-c2nc3c(-c4ccc(N(c5ccc6c(ccc7ccccc76)c5)c5ccc6c(c5)oc5cc7ccccc7cc56)cc4)cccc3o2)cc1 ; c1ccc(-c2nc3c(-c4ccc(N(c5ccc6c(c5)C(c5ccccc5)(c5ccccc5)c5ccccc5-6)c5ccc6c(c5)oc5cc7ccccc7cc56)cc4)cccc3o2)cc1 ; c1ccc(-c2nc3c(-c4ccc(N(c5ccc(-c6ccc7ccccc7c6)cc5)c5ccc6c(c5)oc5cc7ccccc7cc56)cc4)cccc3o2)cc1]

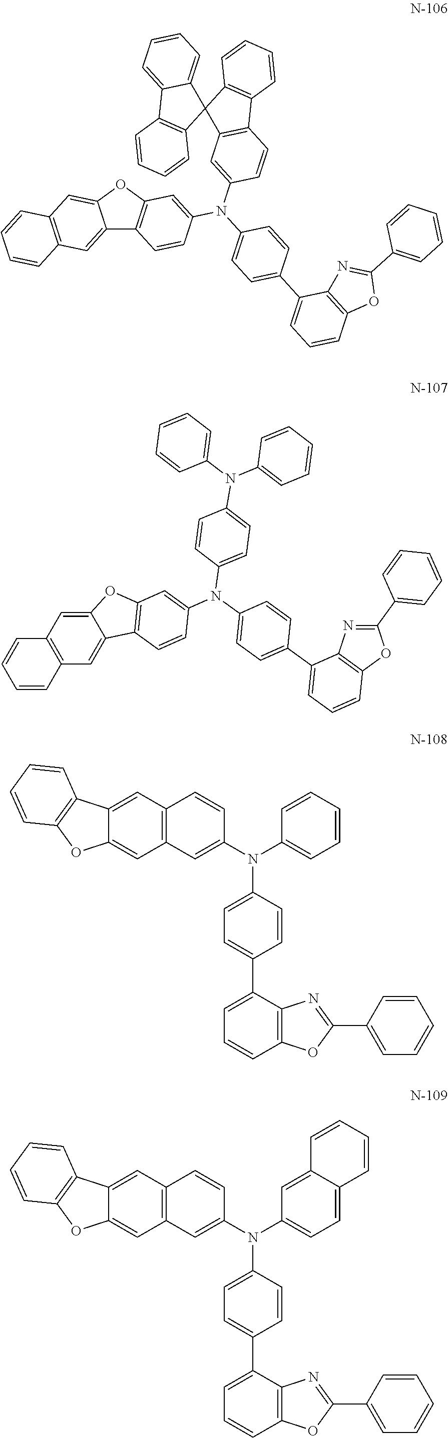 [SMILES: c1ccc(-c2nc3c(-c4ccc(N(c5ccc6c(c5)C5(c7ccccc7-c7ccccc75)c5ccccc5-6)c5ccc6c(c5)oc5cc7ccccc7cc56)cc4)cccc3o2)cc1 ; c1ccc(-c2nc3c(-c4ccc(N(c5ccc(N(c6ccccc6)c6ccccc6)cc5)c5ccc6c(c5)oc5cc7ccccc7cc56)cc4)cccc3o2)cc1 ; c1ccc(-c2nc3c(-c4ccc(N(c5ccccc5)c5ccc6cc7c(cc6c5)oc5ccccc57)cc4)cccc3o2)cc1 ; c1ccc(-c2nc3c(-c4ccc(N(c5ccc6ccccc6c5)c5ccc6cc7c(cc6c5)oc5ccccc57)cc4)cccc3o2)cc1]

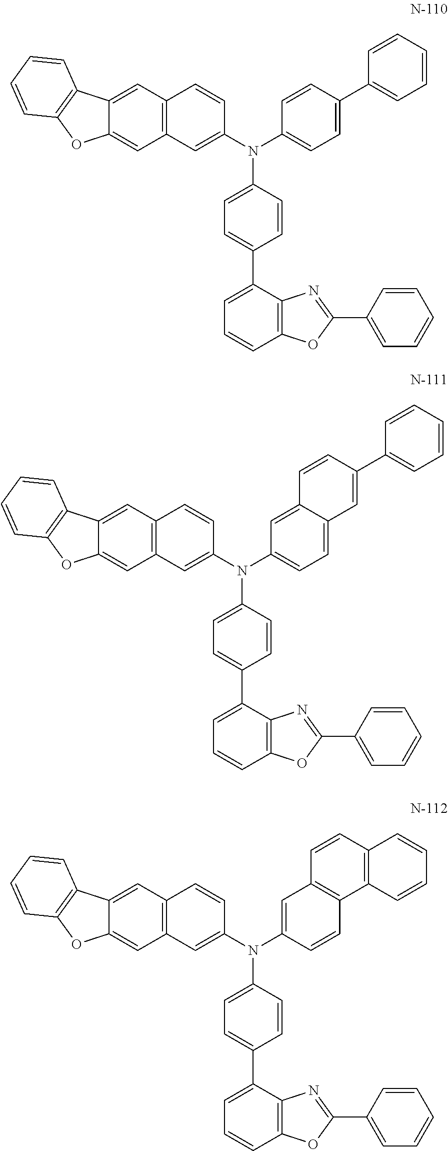 [SMILES: c1ccc(-c2ccc(N(c3ccc(-c4cccc5oc(-c6ccccc6)nc45)cc3)c3ccc4cc5c(cc4c3)oc3ccccc35)cc2)cc1 ; c1ccc(-c2ccc3cc(N(c4ccc(-c5cccc6oc(-c7ccccc7)nc56)cc4)c4ccc5cc6c(cc5c4)oc4ccccc46)ccc3c2)cc1 ; c1ccc(-c2nc3c(-c4ccc(N(c5ccc6cc7c(cc6c5)oc5ccccc57)c5ccc6c(ccc7ccccc76)c5)cc4)cccc3o2)cc1]

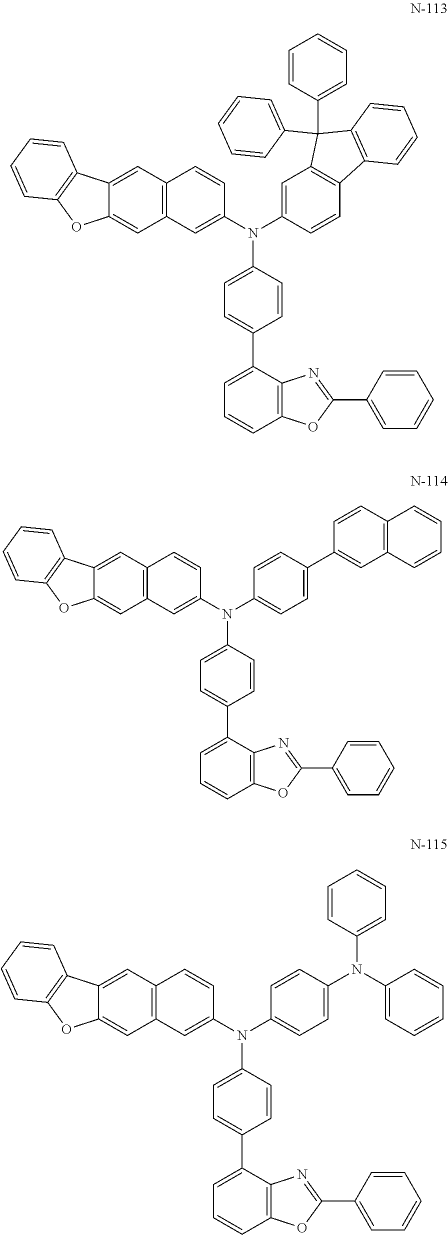 [SMILES: c1ccc(-c2nc3c(-c4ccc(N(c5ccc6c(c5)C(c5ccccc5)(c5ccccc5)c5ccccc5-6)c5ccc6cc7c(cc6c5)oc5ccccc57)cc4)cccc3o2)cc1 ; c1ccc(-c2nc3c(-c4ccc(N(c5ccc(-c6ccc7ccccc7c6)cc5)c5ccc6cc7c(cc6c5)oc5ccccc57)cc4)cccc3o2)cc1 ; c1ccc(-c2nc3c(-c4ccc(N(c5ccc(N(c6ccccc6)c6ccccc6)cc5)c5ccc6cc7c(cc6c5)oc5ccccc57)cc4)cccc3o2)cc1]

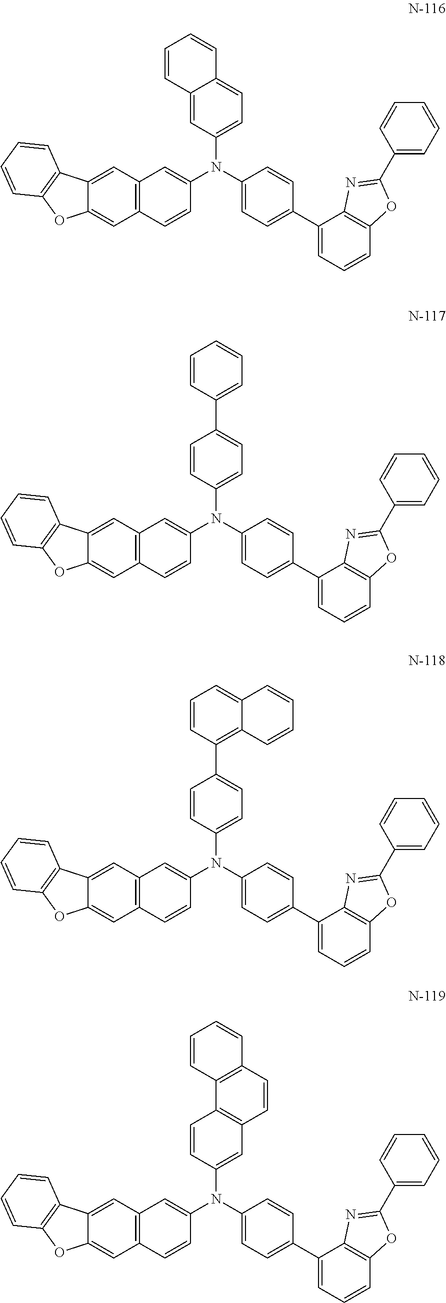 [SMILES: c1ccc(-c2nc3c(-c4ccc(N(c5ccc6ccccc6c5)c5ccc6cc7oc8ccccc8c7cc6c5)cc4)cccc3o2)cc1 ; c1ccc(-c2ccc(N(c3ccc(-c4cccc5oc(-c6ccccc6)nc45)cc3)c3ccc4cc5oc6ccccc6c5cc4c3)cc2)cc1 ; c1ccc(-c2nc3c(-c4ccc(N(c5ccc(-c6cccc7ccccc67)cc5)c5ccc6cc7oc8ccccc8c7cc6c5)cc4)cccc3o2)cc1 ; c1ccc(-c2nc3c(-c4ccc(N(c5ccc6cc7oc8ccccc8c7cc6c5)c5ccc6c(ccc7ccccc76)c5)cc4)cccc3o2)cc1]

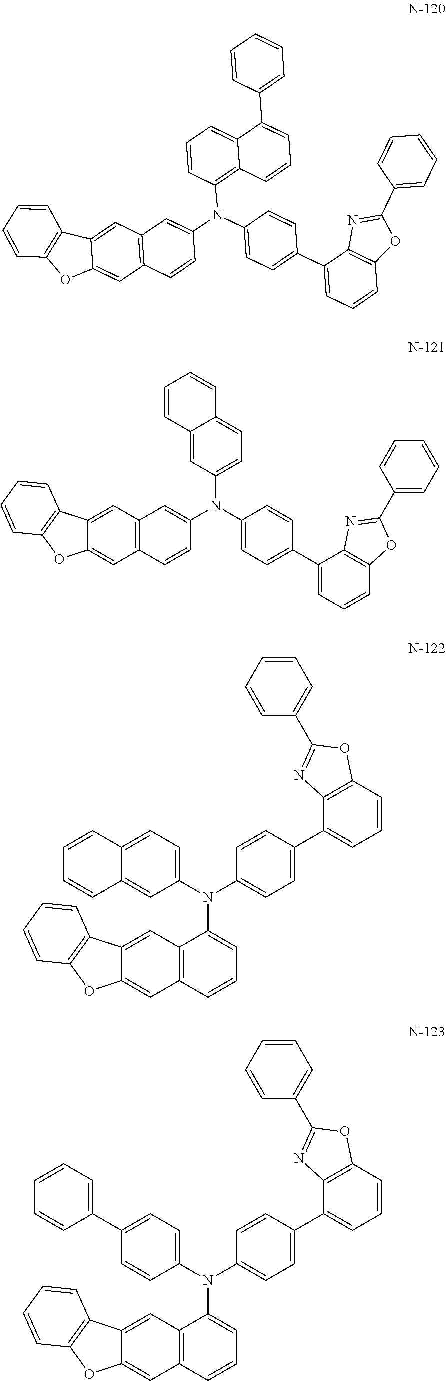 [SMILES: c1ccc(-c2nc3c(-c4ccc(N(c5ccc6cc7oc8ccccc8c7cc6c5)c5cccc6c(-c7ccccc7)cccc56)cc4)cccc3o2)cc1 ; c1ccc(-c2nc3c(-c4ccc(N(c5ccc6ccccc6c5)c5ccc6cc7oc8ccccc8c7cc6c5)cc4)cccc3o2)cc1 ; c1ccc(-c2nc3c(-c4ccc(N(c5ccc6ccccc6c5)c5cccc6cc7oc8ccccc8c7cc56)cc4)cccc3o2)cc1 ; c1ccc(-c2ccc(N(c3ccc(-c4cccc5oc(-c6ccccc6)nc45)cc3)c3cccc4cc5oc6ccccc6c5cc34)cc2)cc1]

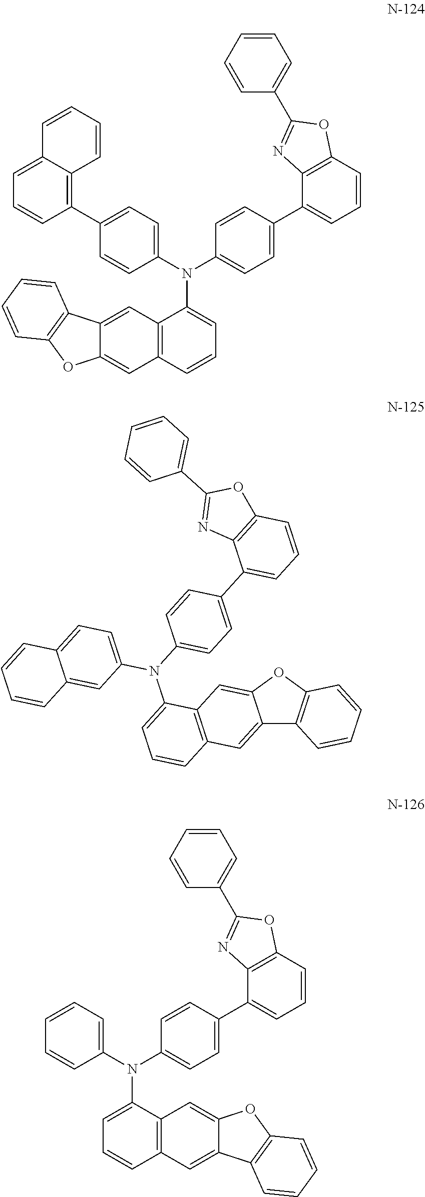 [SMILES: c1ccc(-c2nc3c(-c4ccc(N(c5ccc(-c6cccc7ccccc67)cc5)c5cccc6cc7oc8ccccc8c7cc56)cc4)cccc3o2)cc1 ; c1ccc(-c2nc3c(-c4ccc(N(c5ccc6ccccc6c5)c5cccc6cc7c(cc56)oc5ccccc57)cc4)cccc3o2)cc1 ; c1ccc(-c2nc3c(-c4ccc(N(c5ccccc5)c5cccc6cc7c(cc56)oc5ccccc57)cc4)cccc3o2)cc1]

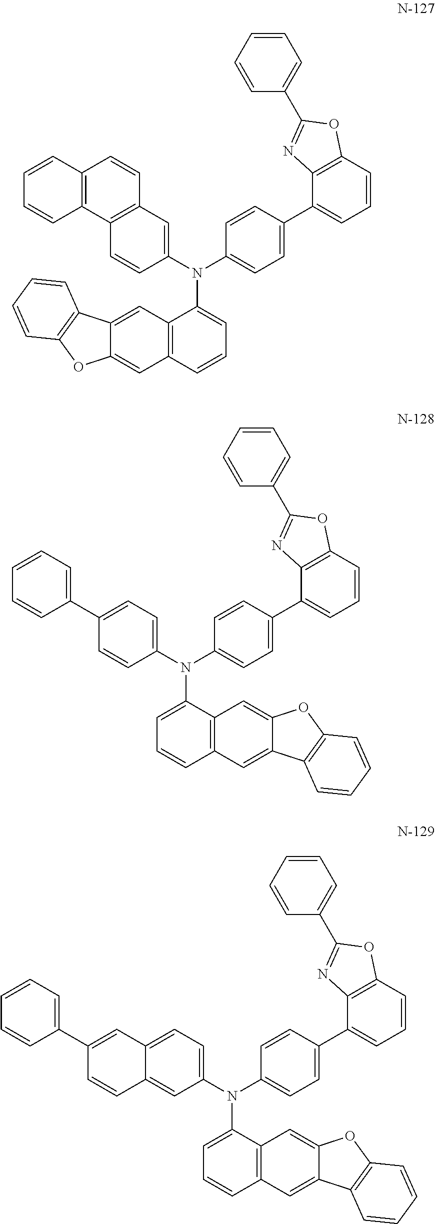 [SMILES: c1ccc(-c2nc3c(-c4ccc(N(c5ccc6c(ccc7ccccc76)c5)c5cccc6cc7oc8ccccc8c7cc56)cc4)cccc3o2)cc1 ; c1ccc(-c2ccc(N(c3ccc(-c4cccc5oc(-c6ccccc6)nc45)cc3)c3cccc4cc5c(cc34)oc3ccccc35)cc2)cc1 ; c1ccc(-c2ccc3cc(N(c4ccc(-c5cccc6oc(-c7ccccc7)nc56)cc4)c4cccc5cc6c(cc45)oc4ccccc46)ccc3c2)cc1]

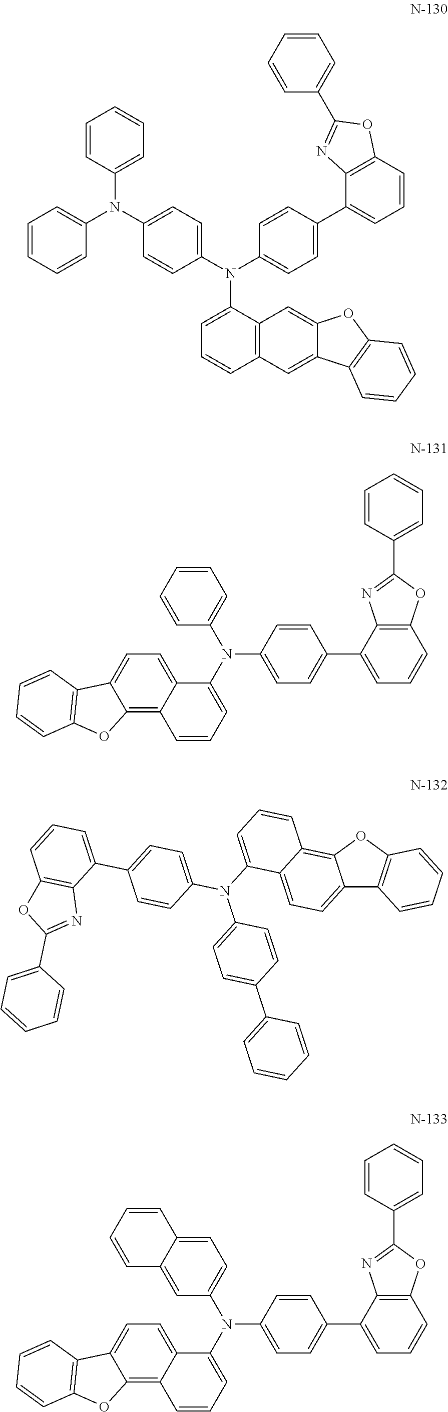 [SMILES: c1ccc(-c2nc3c(-c4ccc(N(c5ccc(N(c6ccccc6)c6ccccc6)cc5)c5cccc6cc7c(cc56)oc5ccccc57)cc4)cccc3o2)cc1 ; c1ccc(-c2nc3c(-c4ccc(N(c5ccccc5)c5cccc6c5ccc5c7ccccc7oc65)cc4)cccc3o2)cc1 ; c1ccc(-c2ccc(N(c3ccc(-c4cccc5oc(-c6ccccc6)nc45)cc3)c3cccc4c3ccc3c5ccccc5oc43)cc2)cc1 ; c1ccc(-c2nc3c(-c4ccc(N(c5ccc6ccccc6c5)c5cccc6c5ccc5c7ccccc7oc65)cc4)cccc3o2)cc1]

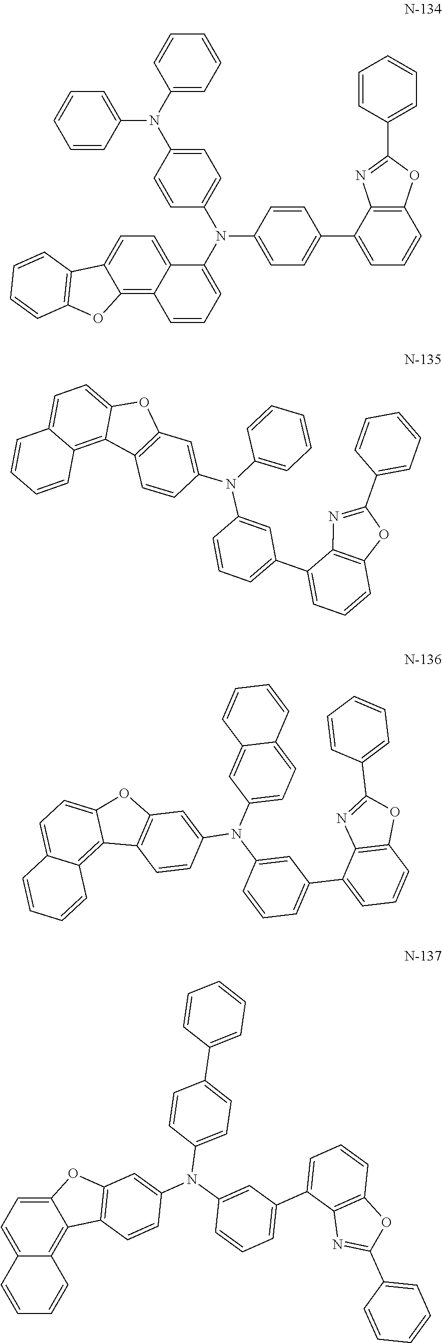 [SMILES: c1ccc(-c2nc3c(-c4ccc(N(c5ccc(N(c6ccccc6)c6ccccc6)cc5)c5cccc6c5ccc5c7ccccc7oc65)cc4)cccc3o2)cc1 ; c1ccc(-c2nc3c(-c4cccc(N(c5ccccc5)c5ccc6c(c5)oc5ccc7ccccc7c56)c4)cccc3o2)cc1 ; c1ccc(-c2nc3c(-c4cccc(N(c5ccc6ccccc6c5)c5ccc6c(c5)oc5ccc7ccccc7c56)c4)cccc3o2)cc1 ; c1ccc(-c2ccc(N(c3cccc(-c4cccc5oc(-c6ccccc6)nc45)c3)c3ccc4c(c3)oc3ccc5ccccc5c34)cc2)cc1]

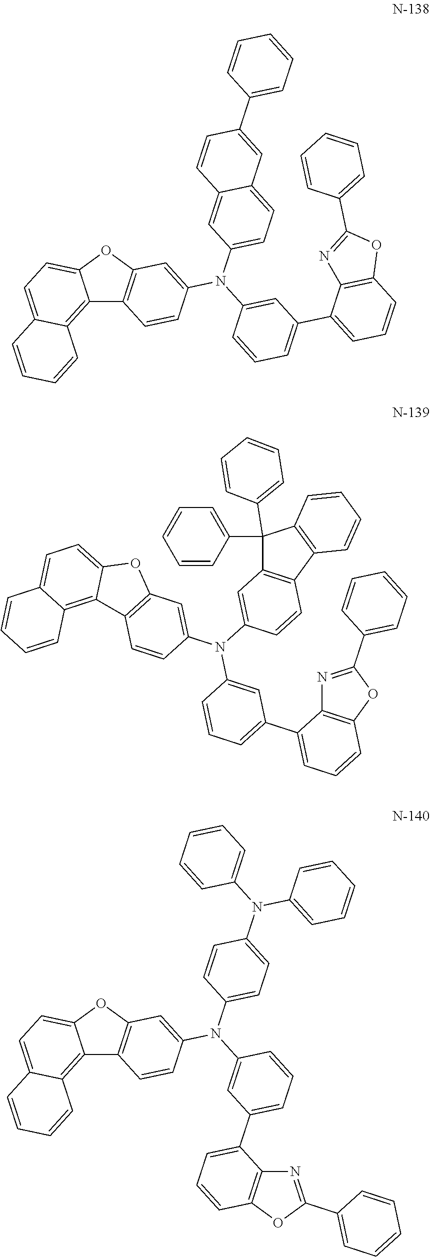 [SMILES: c1ccc(-c2ccc3cc(N(c4cccc(-c5cccc6oc(-c7ccccc7)nc56)c4)c4ccc5c(c4)oc4ccc6ccccc6c45)ccc3c2)cc1 ; c1ccc(-c2nc3c(-c4cccc(N(c5ccc6c(c5)C(c5ccccc5)(c5ccccc5)c5ccccc5-6)c5ccc6c(c5)oc5ccc7ccccc7c56)c4)cccc3o2)cc1 ; c1ccc(-c2nc3c(-c4cccc(N(c5ccc(N(c6ccccc6)c6ccccc6)cc5)c5ccc6c(c5)oc5ccc7ccccc7c56)c4)cccc3o2)cc1]

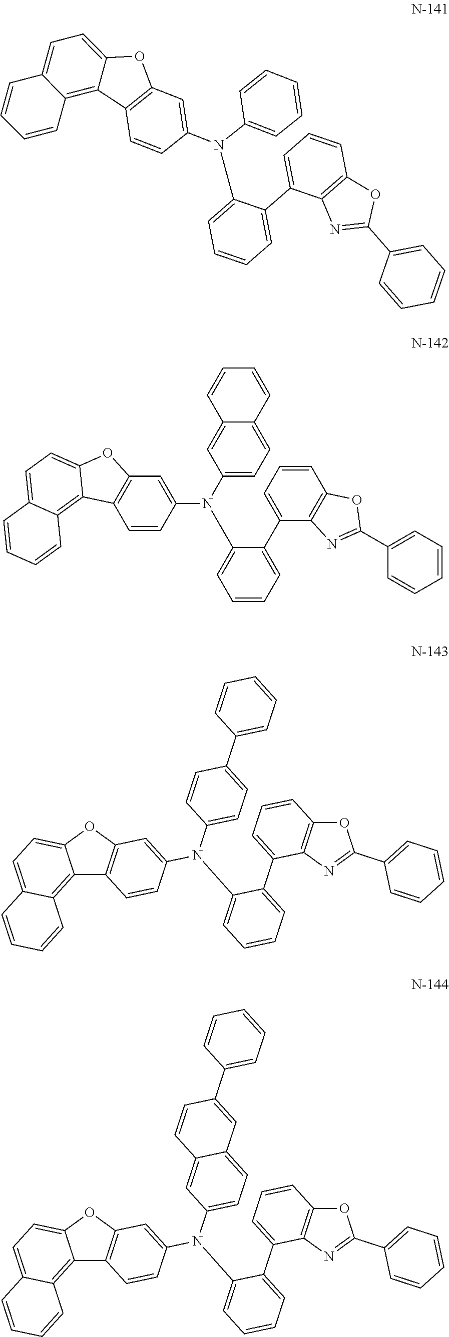 [SMILES: c1ccc(-c2nc3c(-c4ccccc4N(c4ccccc4)c4ccc5c(c4)oc4ccc6ccccc6c45)cccc3o2)cc1 ; c1ccc(-c2nc3c(-c4ccccc4N(c4ccc5ccccc5c4)c4ccc5c(c4)oc4ccc6ccccc6c45)cccc3o2)cc1 ; c1ccc(-c2ccc(N(c3ccc4c(c3)oc3ccc5ccccc5c34)c3ccccc3-c3cccc4oc(-c5ccccc5)nc34)cc2)cc1 ; c1ccc(-c2ccc3cc(N(c4ccc5c(c4)oc4ccc6ccccc6c45)c4ccccc4-c4cccc5oc(-c6ccccc6)nc45)ccc3c2)cc1]

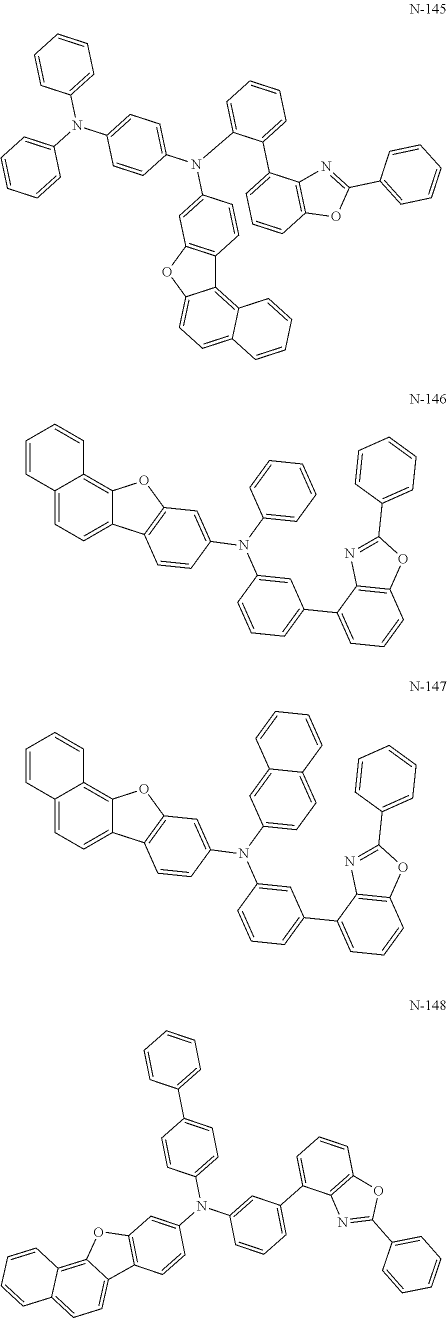 [SMILES: c1ccc(-c2nc3c(-c4ccccc4N(c4ccc(N(c5ccccc5)c5ccccc5)cc4)c4ccc5c(c4)oc4ccc6ccccc6c45)cccc3o2)cc1 ; c1ccc(-c2nc3c(-c4cccc(N(c5ccccc5)c5ccc6c(c5)oc5c7ccccc7ccc65)c4)cccc3o2)cc1 ; c1ccc(-c2nc3c(-c4cccc(N(c5ccc6ccccc6c5)c5ccc6c(c5)oc5c7ccccc7ccc65)c4)cccc3o2)cc1 ; c1ccc(-c2ccc(N(c3cccc(-c4cccc5oc(-c6ccccc6)nc45)c3)c3ccc4c(c3)oc3c5ccccc5ccc43)cc2)cc1]

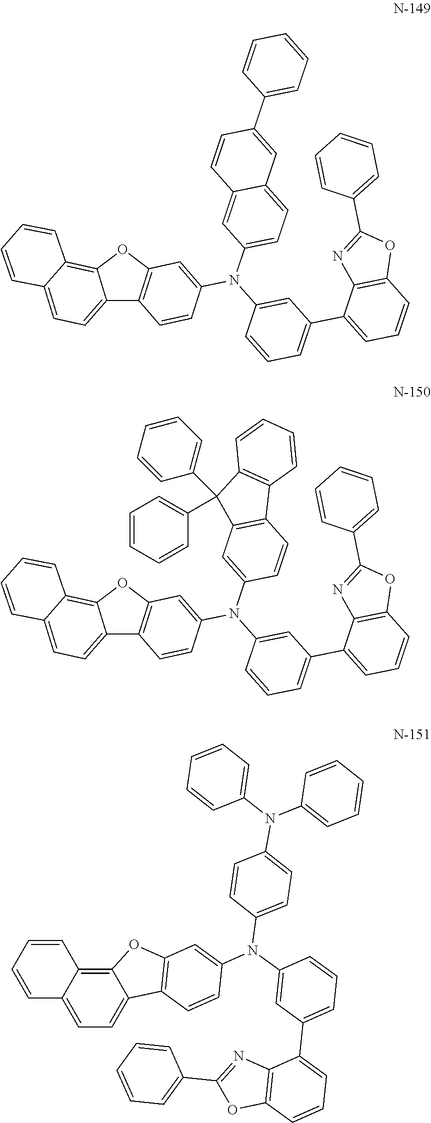 [SMILES: c1ccc(-c2ccc3cc(N(c4cccc(-c5cccc6oc(-c7ccccc7)nc56)c4)c4ccc5c(c4)oc4c6ccccc6ccc54)ccc3c2)cc1 ; c1ccc(-c2nc3c(-c4cccc(N(c5ccc6c(c5)C(c5ccccc5)(c5ccccc5)c5ccccc5-6)c5ccc6c(c5)oc5c7ccccc7ccc65)c4)cccc3o2)cc1 ; c1ccc(-c2nc3c(-c4cccc(N(c5ccc(N(c6ccccc6)c6ccccc6)cc5)c5ccc6c(c5)oc5c7ccccc7ccc65)c4)cccc3o2)cc1]

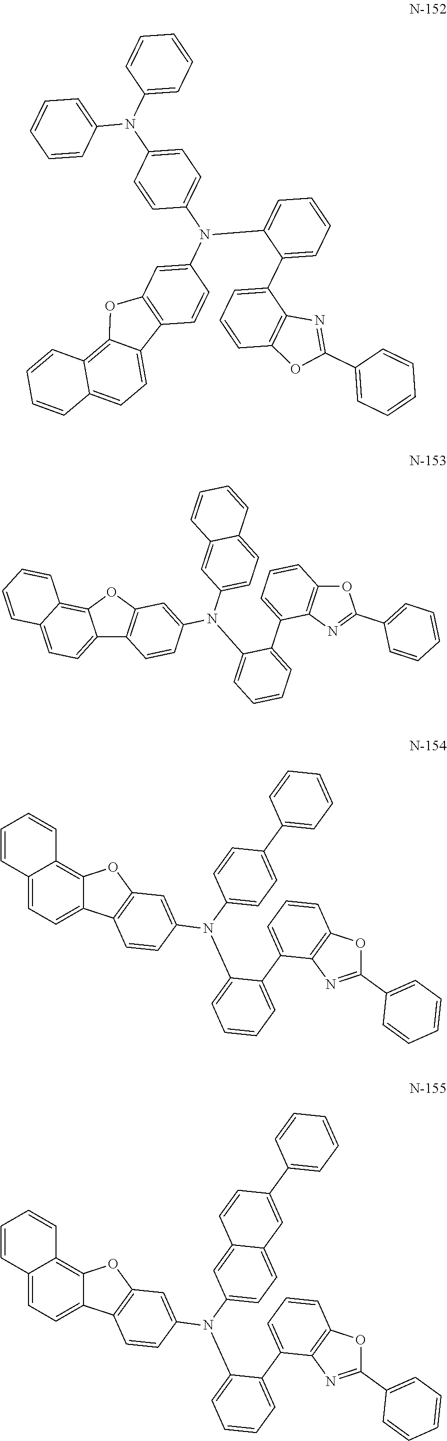 [SMILES: c1ccc(-c2nc3c(-c4ccccc4N(c4ccc(N(c5ccccc5)c5ccccc5)cc4)c4ccc5c(c4)oc4c6ccccc6ccc54)cccc3o2)cc1 ; c1ccc(-c2nc3c(-c4ccccc4N(c4ccc5ccccc5c4)c4ccc5c(c4)oc4c6ccccc6ccc54)cccc3o2)cc1 ; c1ccc(-c2ccc(N(c3ccc4c(c3)oc3c5ccccc5ccc43)c3ccccc3-c3cccc4oc(-c5ccccc5)nc34)cc2)cc1 ; c1ccc(-c2ccc3cc(N(c4ccc5c(c4)oc4c6ccccc6ccc54)c4ccccc4-c4cccc5oc(-c6ccccc6)nc45)ccc3c2)cc1]

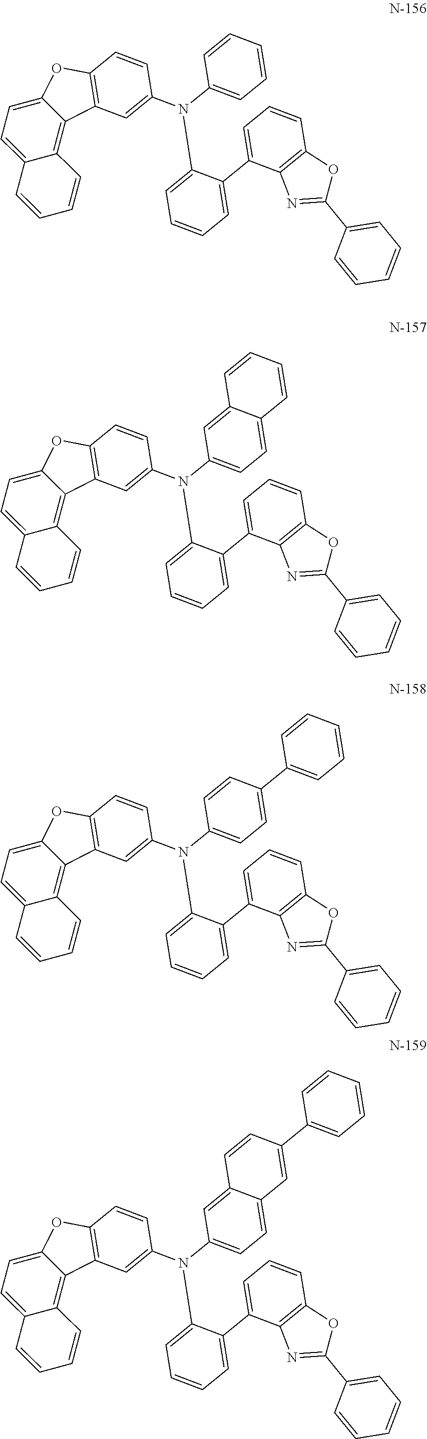 [SMILES: c1ccc(-c2nc3c(-c4ccccc4N(c4ccccc4)c4ccc5oc6ccc7ccccc7c6c5c4)cccc3o2)cc1 ; c1ccc(-c2nc3c(-c4ccccc4N(c4ccc5ccccc5c4)c4ccc5oc6ccc7ccccc7c6c5c4)cccc3o2)cc1 ; c1ccc(-c2ccc(N(c3ccc4oc5ccc6ccccc6c5c4c3)c3ccccc3-c3cccc4oc(-c5ccccc5)nc34)cc2)cc1 ; c1ccc(-c2ccc3cc(N(c4ccc5oc6ccc7ccccc7c6c5c4)c4ccccc4-c4cccc5oc(-c6ccccc6)nc45)ccc3c2)cc1]

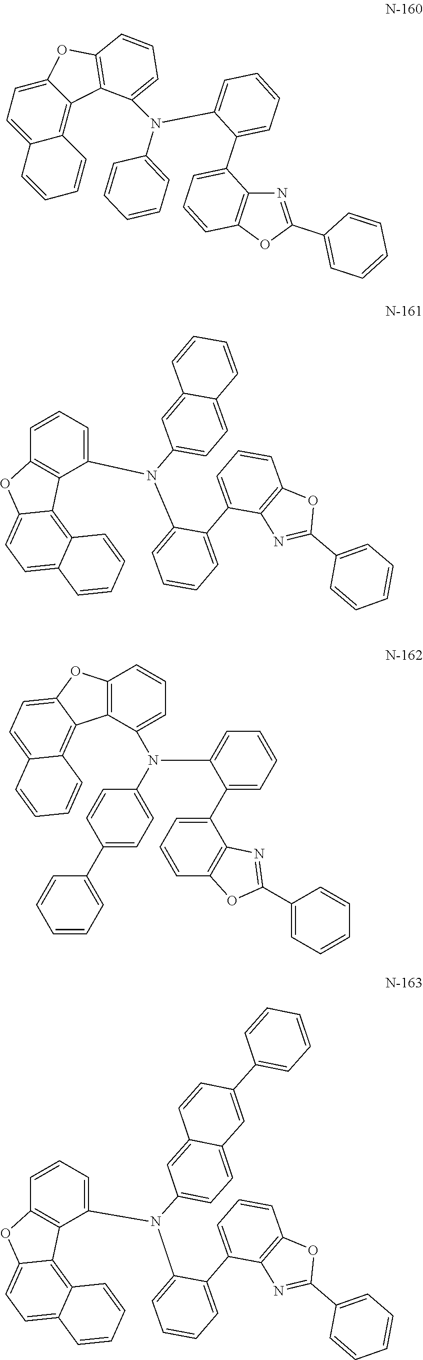 [SMILES: c1ccc(-c2nc3c(-c4ccccc4N(c4ccccc4)c4cccc5oc6ccc7ccccc7c6c45)cccc3o2)cc1 ; c1ccc(-c2nc3c(-c4ccccc4N(c4ccc5ccccc5c4)c4cccc5oc6ccc7ccccc7c6c45)cccc3o2)cc1 ; c1ccc(-c2ccc(N(c3ccccc3-c3cccc4oc(-c5ccccc5)nc34)c3cccc4oc5ccc6ccccc6c5c34)cc2)cc1 ; c1ccc(-c2ccc3cc(N(c4ccccc4-c4cccc5oc(-c6ccccc6)nc45)c4cccc5oc6ccc7ccccc7c6c45)ccc3c2)cc1]

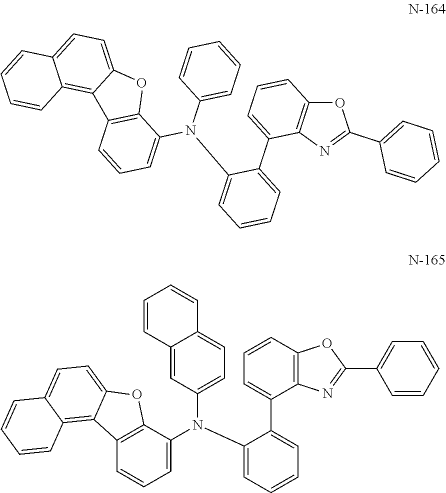 [SMILES: c1ccc(-c2nc3c(-c4ccccc4N(c4ccccc4)c4cccc5c4oc4ccc6ccccc6c45)cccc3o2)cc1 ; c1ccc(-c2nc3c(-c4ccccc4N(c4ccc5ccccc5c4)c4cccc5c4oc4ccc6ccccc6c45)cccc3o2)cc1]

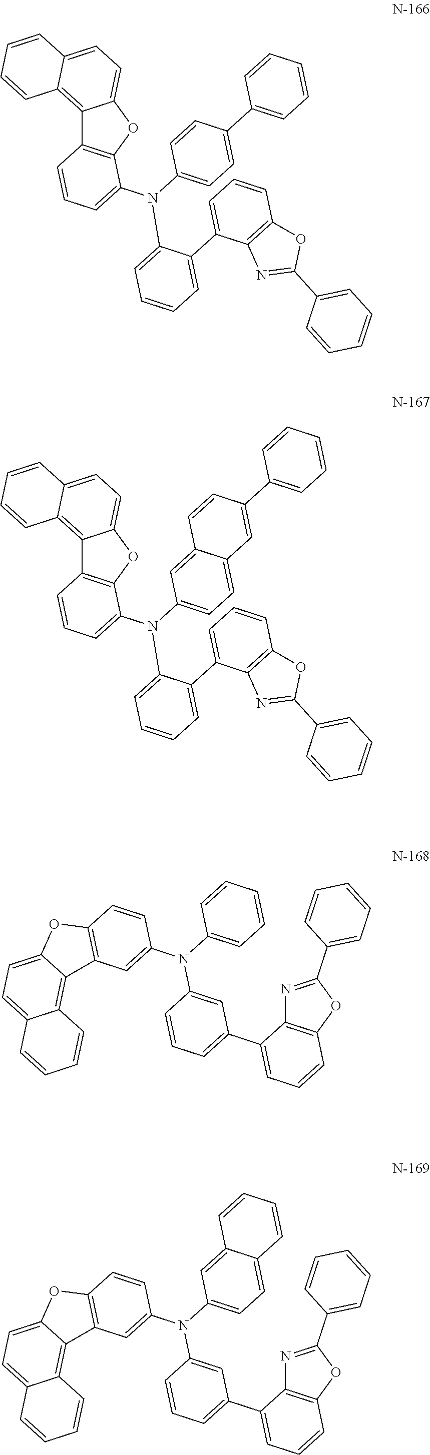 [SMILES: c1ccc(-c2ccc(N(c3ccccc3-c3cccc4oc(-c5ccccc5)nc34)c3cccc4c3oc3ccc5ccccc5c34)cc2)cc1 ; c1ccc(-c2ccc3cc(N(c4ccccc4-c4cccc5oc(-c6ccccc6)nc45)c4cccc5c4oc4ccc6ccccc6c45)ccc3c2)cc1 ; c1ccc(-c2nc3c(-c4cccc(N(c5ccccc5)c5ccc6oc7ccc8ccccc8c7c6c5)c4)cccc3o2)cc1 ; c1ccc(-c2nc3c(-c4cccc(N(c5ccc6ccccc6c5)c5ccc6oc7ccc8ccccc8c7c6c5)c4)cccc3o2)cc1]

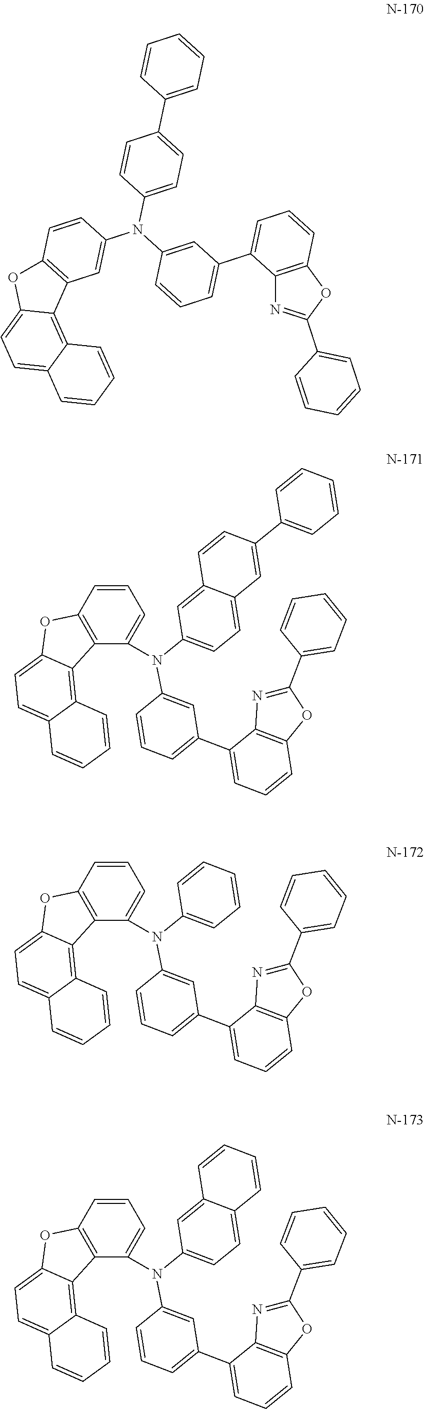 [SMILES: c1ccc(-c2ccc(N(c3cccc(-c4cccc5oc(-c6ccccc6)nc45)c3)c3ccc4oc5ccc6ccccc6c5c4c3)cc2)cc1 ; c1ccc(-c2ccc3cc(N(c4cccc(-c5cccc6oc(-c7ccccc7)nc56)c4)c4cccc5oc6ccc7ccccc7c6c45)ccc3c2)cc1 ; c1ccc(-c2nc3c(-c4cccc(N(c5ccccc5)c5cccc6oc7ccc8ccccc8c7c56)c4)cccc3o2)cc1 ; c1ccc(-c2nc3c(-c4cccc(N(c5ccc6ccccc6c5)c5cccc6oc7ccc8ccccc8c7c56)c4)cccc3o2)cc1]

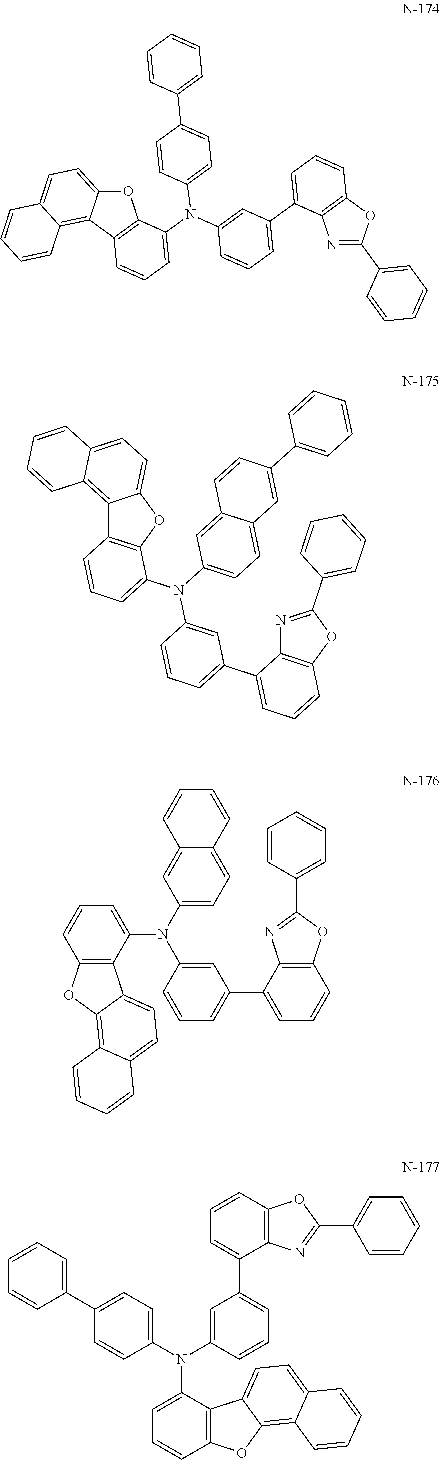 [SMILES: c1ccc(-c2ccc(N(c3cccc(-c4cccc5oc(-c6ccccc6)nc45)c3)c3cccc4c3oc3ccc5ccccc5c34)cc2)cc1 ; c1ccc(-c2ccc3cc(N(c4cccc(-c5cccc6oc(-c7ccccc7)nc56)c4)c4cccc5c4oc4ccc6ccccc6c45)ccc3c2)cc1 ; c1ccc(-c2nc3c(-c4cccc(N(c5ccc6ccccc6c5)c5cccc6oc7c8ccccc8ccc7c56)c4)cccc3o2)cc1 ; c1ccc(-c2ccc(N(c3cccc(-c4cccc5oc(-c6ccccc6)nc45)c3)c3cccc4oc5c6ccccc6ccc5c34)cc2)cc1]

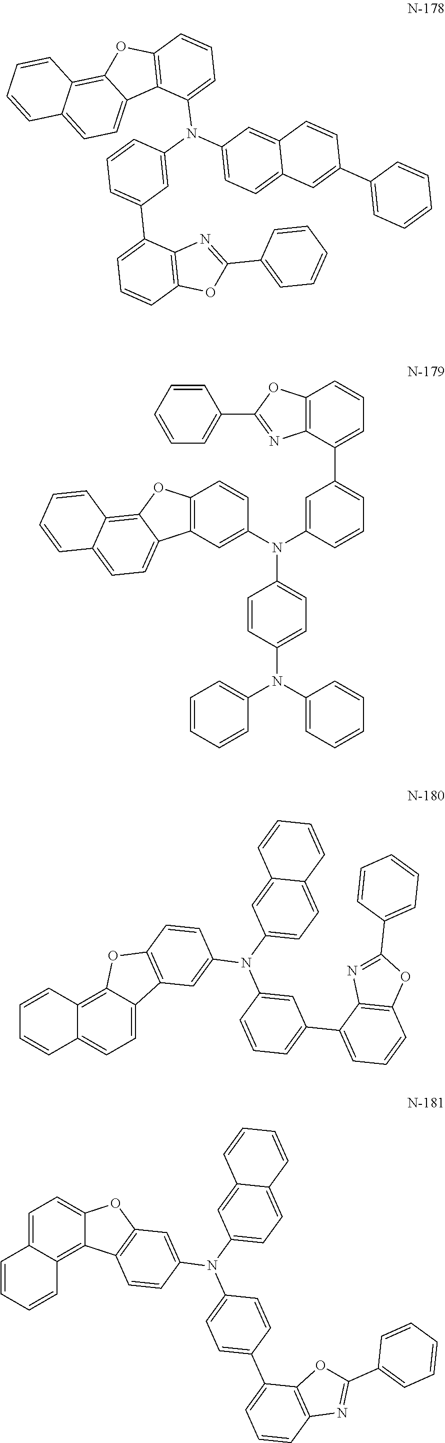 [SMILES: c1ccc(-c2ccc3cc(N(c4cccc(-c5cccc6oc(-c7ccccc7)nc56)c4)c4cccc5oc6c7ccccc7ccc6c45)ccc3c2)cc1 ; c1ccc(-c2nc3c(-c4cccc(N(c5ccc(N(c6ccccc6)c6ccccc6)cc5)c5ccc6oc7c8ccccc8ccc7c6c5)c4)cccc3o2)cc1 ; c1ccc(-c2nc3c(-c4cccc(N(c5ccc6ccccc6c5)c5ccc6oc7c8ccccc8ccc7c6c5)c4)cccc3o2)cc1 ; c1ccc(-c2nc3cccc(-c4ccc(N(c5ccc6ccccc6c5)c5ccc6c(c5)oc5ccc7ccccc7c56)cc4)c3o2)cc1]

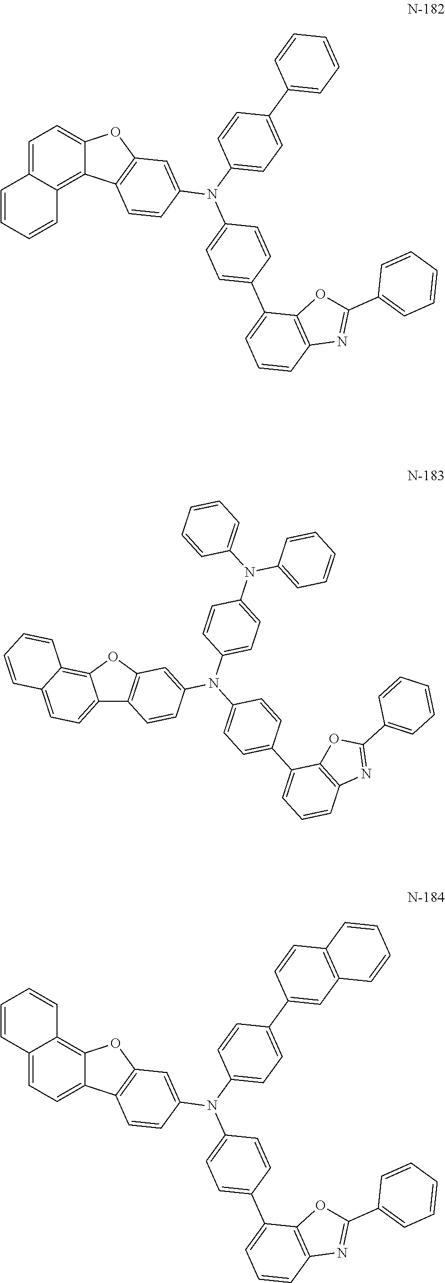 [SMILES: c1ccc(-c2ccc(N(c3ccc(-c4cccc5nc(-c6ccccc6)oc45)cc3)c3ccc4c(c3)oc3ccc5ccccc5c34)cc2)cc1 ; c1ccc(-c2nc3cccc(-c4ccc(N(c5ccc(N(c6ccccc6)c6ccccc6)cc5)c5ccc6c(c5)oc5c7ccccc7ccc65)cc4)c3o2)cc1 ; c1ccc(-c2nc3cccc(-c4ccc(N(c5ccc(-c6ccc7ccccc7c6)cc5)c5ccc6c(c5)oc5c7ccccc7ccc65)cc4)c3o2)cc1]

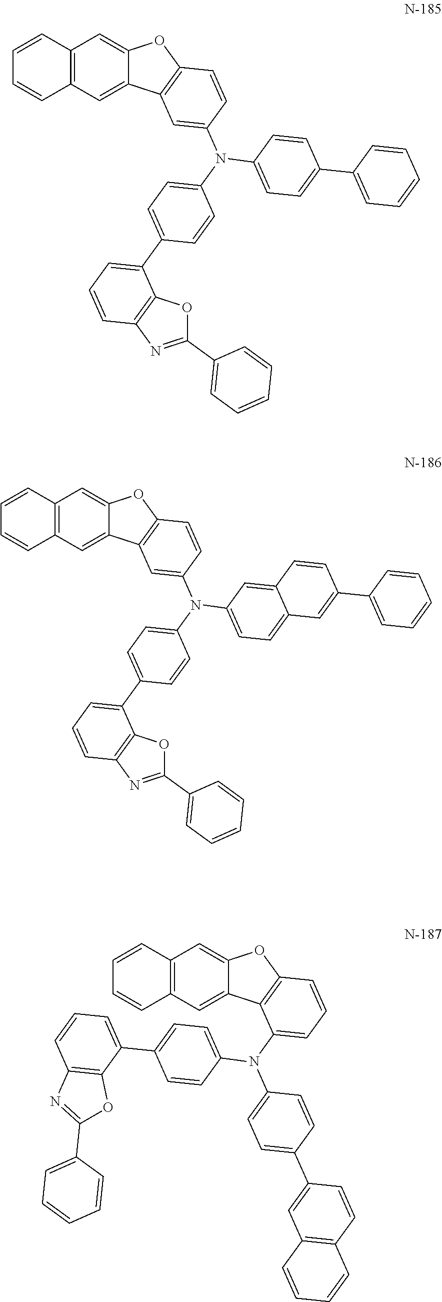 [SMILES: c1ccc(-c2ccc(N(c3ccc(-c4cccc5nc(-c6ccccc6)oc45)cc3)c3ccc4oc5cc6ccccc6cc5c4c3)cc2)cc1 ; c1ccc(-c2ccc3cc(N(c4ccc(-c5cccc6nc(-c7ccccc7)oc56)cc4)c4ccc5oc6cc7ccccc7cc6c5c4)ccc3c2)cc1 ; c1ccc(-c2nc3cccc(-c4ccc(N(c5ccc(-c6ccc7ccccc7c6)cc5)c5cccc6oc7cc8ccccc8cc7c56)cc4)c3o2)cc1]

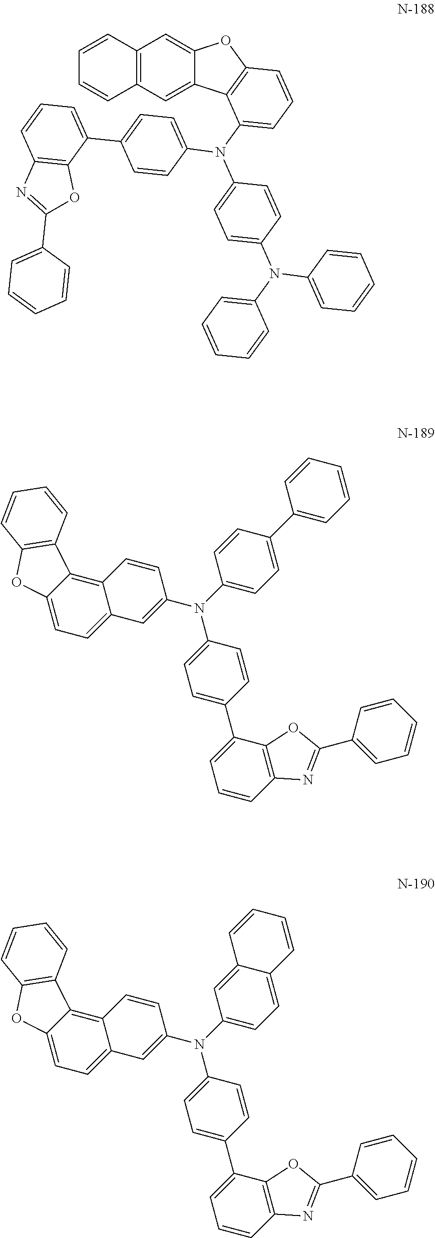 [SMILES: c1ccc(-c2nc3cccc(-c4ccc(N(c5ccc(N(c6ccccc6)c6ccccc6)cc5)c5cccc6oc7cc8ccccc8cc7c56)cc4)c3o2)cc1 ; c1ccc(-c2ccc(N(c3ccc(-c4cccc5nc(-c6ccccc6)oc45)cc3)c3ccc4c(ccc5oc6ccccc6c54)c3)cc2)cc1 ; c1ccc(-c2nc3cccc(-c4ccc(N(c5ccc6ccccc6c5)c5ccc6c(ccc7oc8ccccc8c76)c5)cc4)c3o2)cc1]

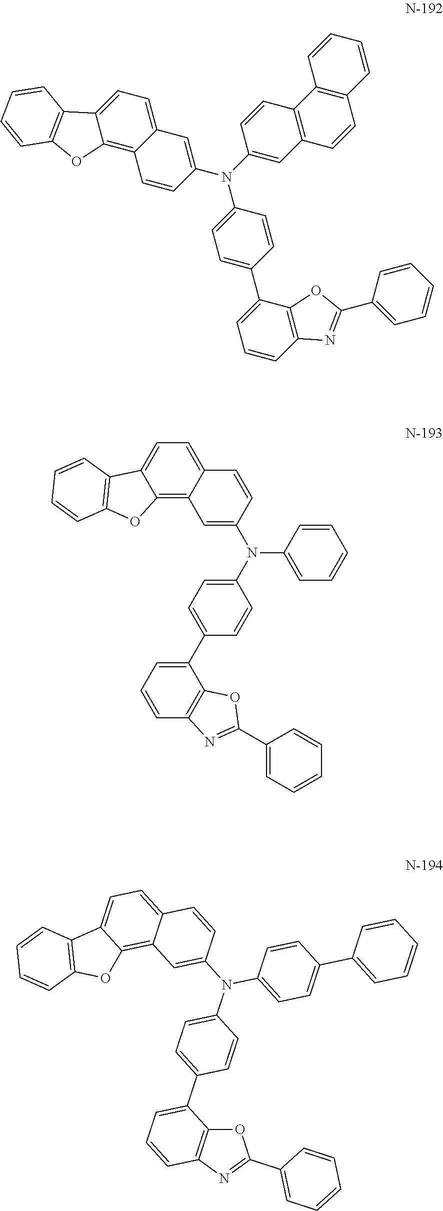 [SMILES: c1ccc(-c2nc3cccc(-c4ccc(N(c5ccc6c(ccc7ccccc76)c5)c5ccc6c(ccc7c8ccccc8oc67)c5)cc4)c3o2)cc1 ; c1ccc(-c2nc3cccc(-c4ccc(N(c5ccccc5)c5ccc6ccc7c8ccccc8oc7c6c5)cc4)c3o2)cc1 ; c1ccc(-c2ccc(N(c3ccc(-c4cccc5nc(-c6ccccc6)oc45)cc3)c3ccc4ccc5c6ccccc6oc5c4c3)cc2)cc1]

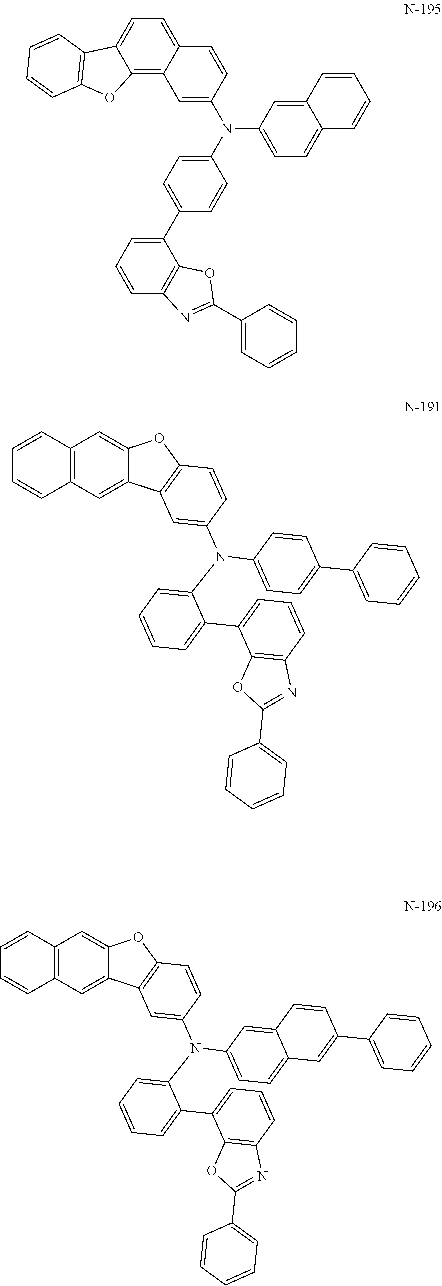 [SMILES: c1ccc(-c2nc3cccc(-c4ccc(N(c5ccc6ccccc6c5)c5ccc6ccc7c8ccccc8oc7c6c5)cc4)c3o2)cc1 ; c1ccc(-c2ccc(N(c3ccc4oc5cc6ccccc6cc5c4c3)c3ccccc3-c3cccc4nc(-c5ccccc5)oc34)cc2)cc1 ; c1ccc(-c2ccc3cc(N(c4ccc5oc6cc7ccccc7cc6c5c4)c4ccccc4-c4cccc5nc(-c6ccccc6)oc45)ccc3c2)cc1]

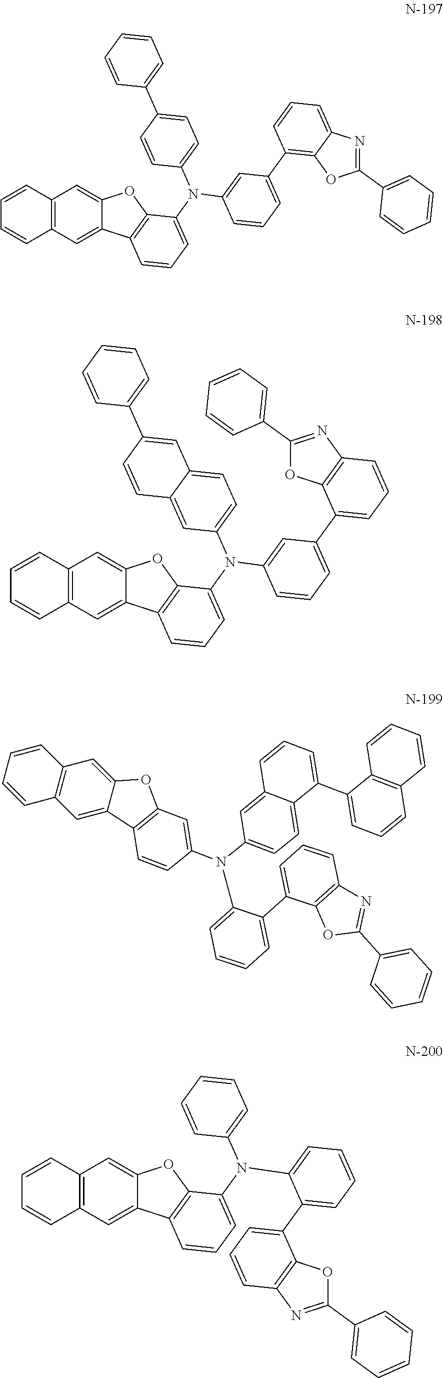 [SMILES: c1ccc(-c2ccc(N(c3cccc(-c4cccc5nc(-c6ccccc6)oc45)c3)c3cccc4c3oc3cc5ccccc5cc34)cc2)cc1 ; c1ccc(-c2ccc3cc(N(c4cccc(-c5cccc6nc(-c7ccccc7)oc56)c4)c4cccc5c4oc4cc6ccccc6cc45)ccc3c2)cc1 ; c1ccc(-c2nc3cccc(-c4ccccc4N(c4ccc5c(-c6cccc7ccccc67)cccc5c4)c4ccc5c(c4)oc4cc6ccccc6cc45)c3o2)cc1 ; c1ccc(-c2nc3cccc(-c4ccccc4N(c4ccccc4)c4cccc5c4oc4cc6ccccc6cc45)c3o2)cc1]

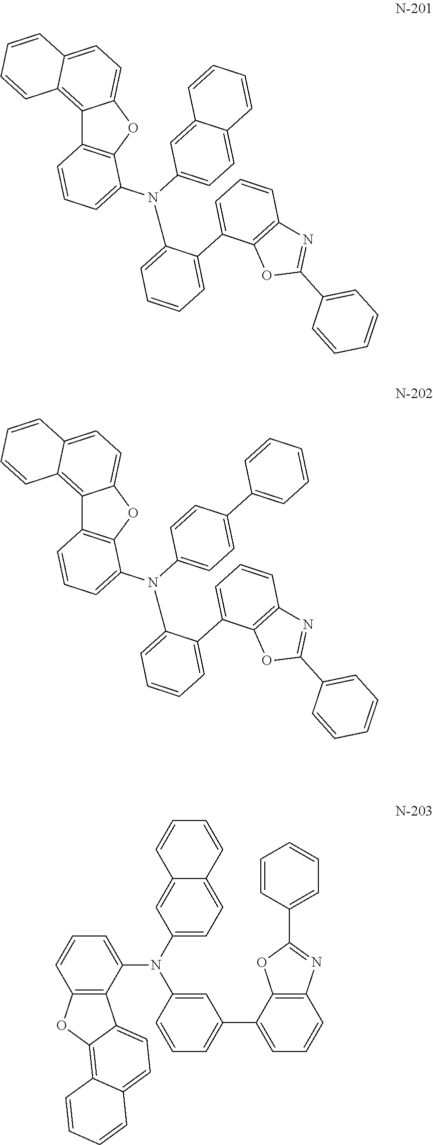 [SMILES: c1ccc(-c2nc3cccc(-c4ccccc4N(c4ccc5ccccc5c4)c4cccc5c4oc4ccc6ccccc6c45)c3o2)cc1 ; c1ccc(-c2ccc(N(c3ccccc3-c3cccc4nc(-c5ccccc5)oc34)c3cccc4c3oc3ccc5ccccc5c34)cc2)cc1 ; c1ccc(-c2nc3cccc(-c4cccc(N(c5ccc6ccccc6c5)c5cccc6oc7c8ccccc8ccc7c56)c4)c3o2)cc1]

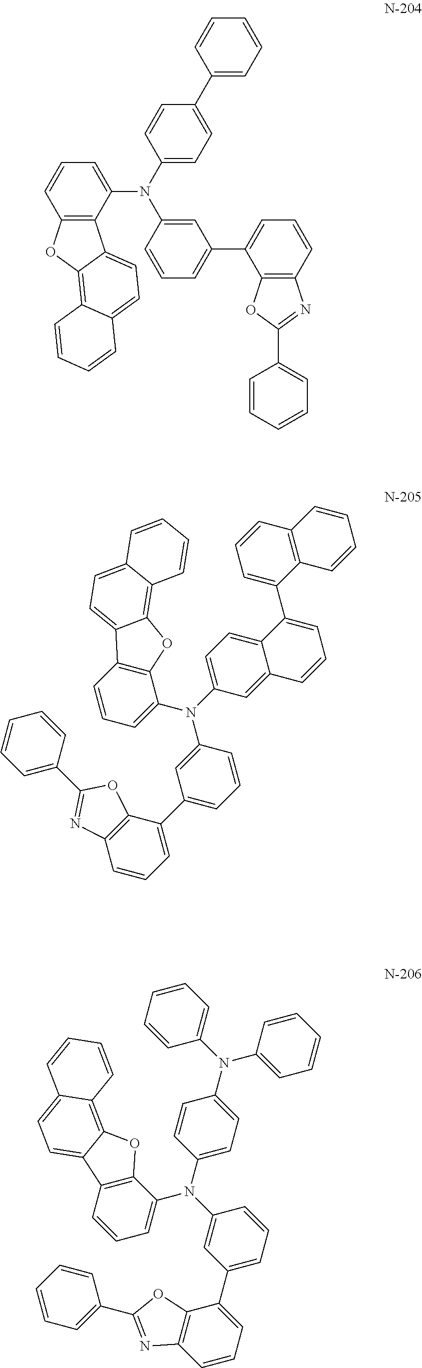 [SMILES: c1ccc(-c2ccc(N(c3cccc(-c4cccc5nc(-c6ccccc6)oc45)c3)c3cccc4oc5c6ccccc6ccc5c34)cc2)cc1 ; c1ccc(-c2nc3cccc(-c4cccc(N(c5ccc6c(-c7cccc8ccccc78)cccc6c5)c5cccc6c5oc5c7ccccc7ccc65)c4)c3o2)cc1 ; c1ccc(-c2nc3cccc(-c4cccc(N(c5ccc(N(c6ccccc6)c6ccccc6)cc5)c5cccc6c5oc5c7ccccc7ccc65)c4)c3o2)cc1]

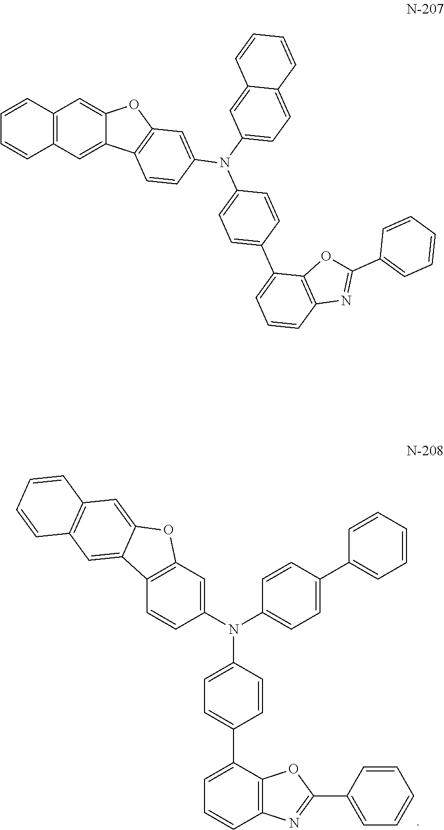 [SMILES: c1ccc(-c2nc3cccc(-c4ccc(N(c5ccc6ccccc6c5)c5ccc6c(c5)oc5cc7ccccc7cc56)cc4)c3o2)cc1 ; c1ccc(-c2ccc(N(c3ccc(-c4cccc5nc(-c6ccccc6)oc45)cc3)c3ccc4c(c3)oc3cc5ccccc5cc34)cc2)cc1]

Optionally, in the Formula (2), X¹-X¹⁴ are all selected from CR, and R is defined as that described above;
optionally, any one of X¹-X⁶ is selected from N, and the others are CR, and R is defined as that described above;
optionally, any one of X¹-X⁶ is selected from N, and the others are CR; any one of X⁷-X¹⁴ is selected from N, and the others are CR, and R is defined as in claim 1;
optionally, R is selected from the group consisting of hydrogen, deuterium, phenyl, and naphthyl;
Ar1 and Ar2 are each independently selected from the group consisting of substituted or unsubstituted C6-C15 aryl, substituted or unsubstituted C3-C20 heteroaryl;
the substituents in the substituted C6-C15 aryl and the substituted C3-C20 heteroaryl are selected from the group consisting of deuterium, halogen, cyano, C1-C6 alkyl, C3-C30 cycloalkyl, C6-C30 aryl, C3-C30 heteroaryl, C6-C60 arylamine group and C3-C60 heteroarylamine, or a combination of two thereof;
optionally, Ar1 and Ar2 are each independently selected from a substituted or unsubstituted A group;
the A group includes phenyl, naphthyl, biphenyl, terphenyl, phenanthryl, fluoranthenyl, triphenylene, fluorenyl, dimethylfluorenyl, diphenylfluorenyl, spirobifluorenyl, benzodimethylfluorenyl, benzodiphenylfluorenyl, benzospirobifluorenyl, benzofuranyl, dibenzofuranyl, naphthobenzofuranyl, dinaphthofuranyl, benzothienyl, dibenzothienyl, naphthobenzothienyl, carbazolyl, phenylcarbazolyl, benzophenylcarbazolyl, dibenzophenylcarbazolyl, biphenylcarbazolyl, phenanthrenylbenzofuranyl, dibenzofuranofuranyl, and phenylcarbazolylbenzofuranyl;
wherein the substituent of the substituted A group is selected from the group consisting of deuterium, halogen, cyano, C1-C6 alkyl, C3-C30 cycloalkyl, C6-C30 aryl, C3-C30 heteroaryl, C6-C60 arylamine group and C3-C60 heteroarylamine group, or a combination of two thereof;
optionally, Ar1 and Ar2 are each independently selected from the group consisting of phenyl, naphthyl, biphenyl, terphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, dibenzofuranyl, naphthobenzofuranyl, dibenzothiophenyl, naphthobenzothiophenyl, carbazolyl, phenylcarbazolyl, benzocarbazolyl, and dibenzocarbazolyl;
optionally, L is independently selected from a connecting bond and substituted or unsubstituted C6-C18 arylene;
optionally, L is selected from a connecting bond and phenylene.

Optionally, the second compound has a structure as shown in any one of the following Formulas 2-1 to 2-28:

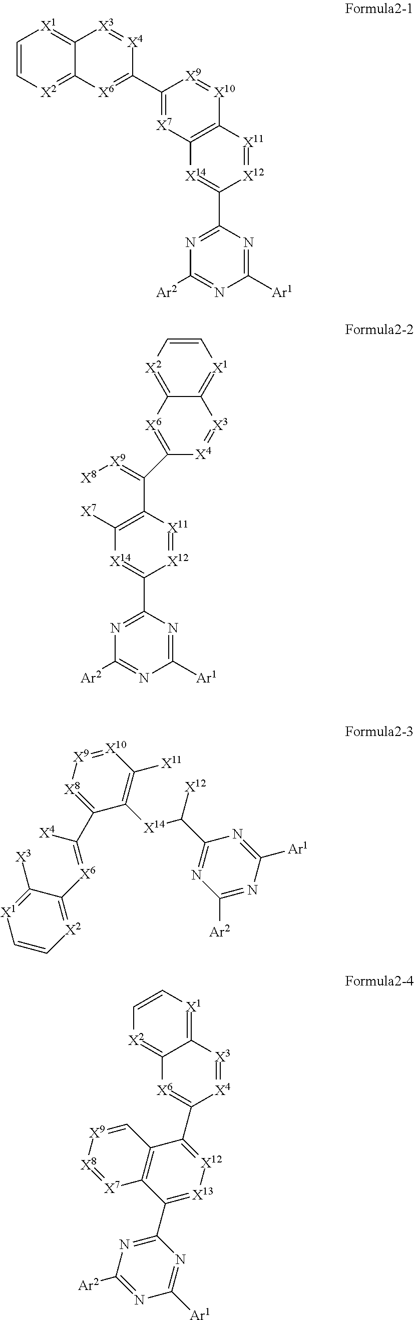

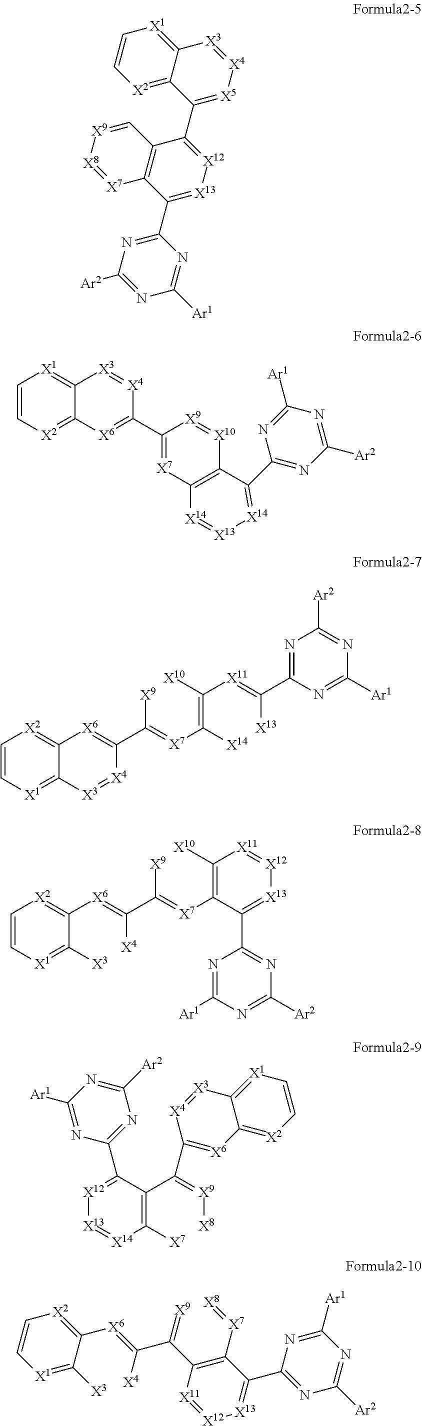

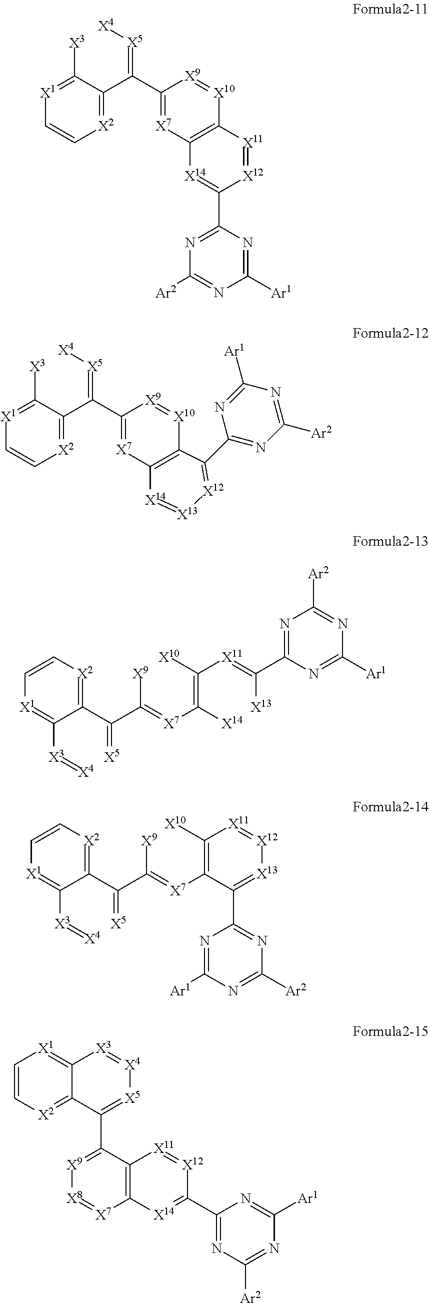

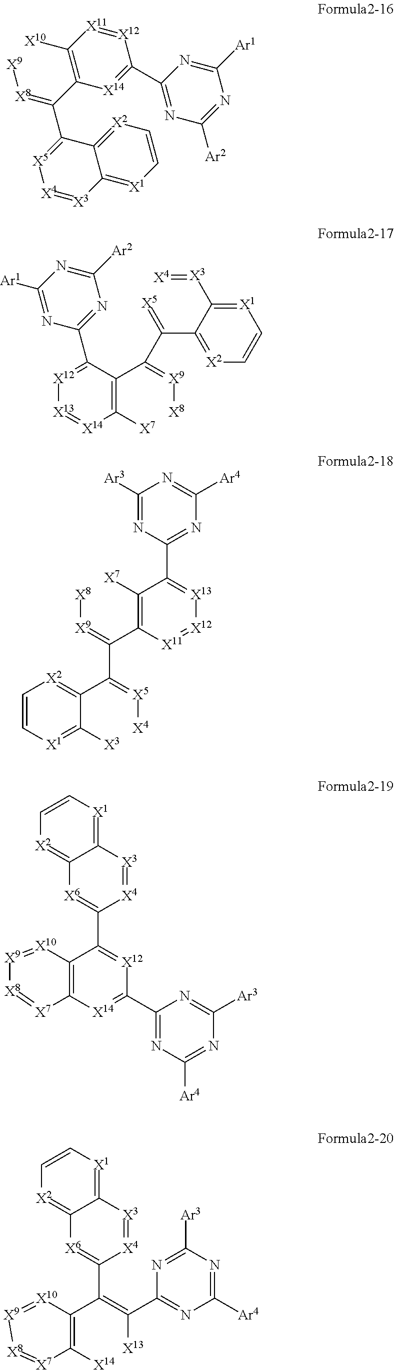

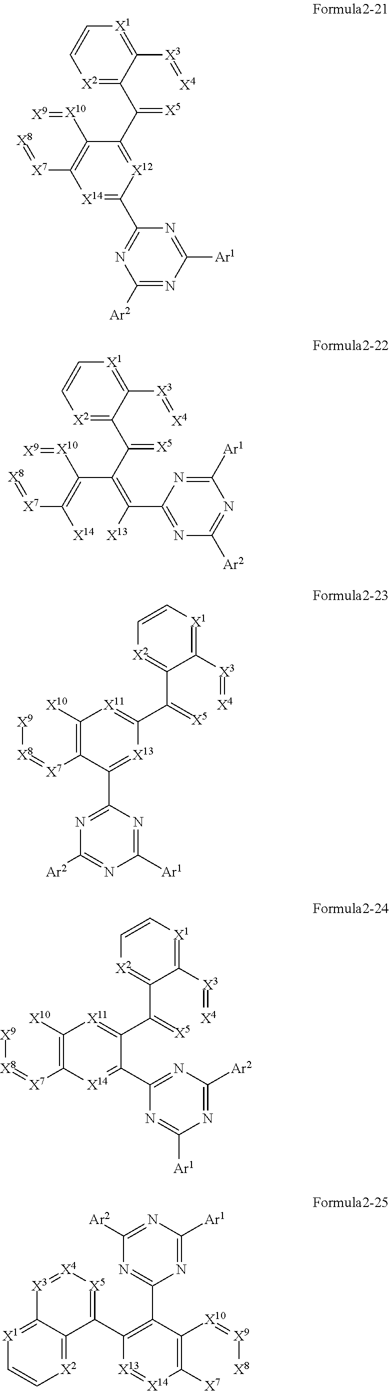

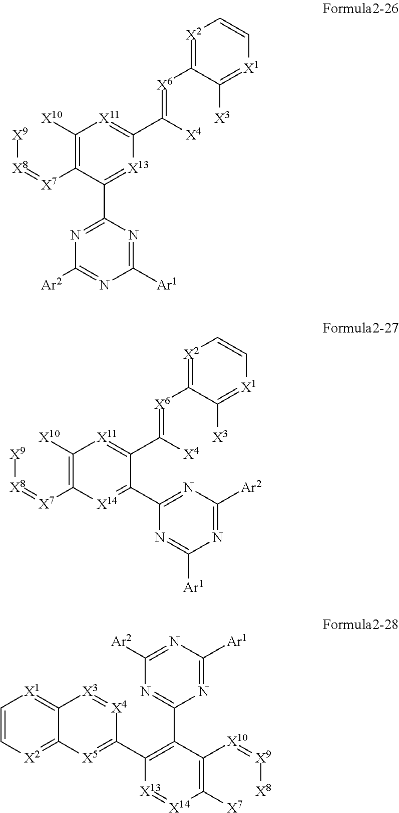

optionally, the definitions of X¹-X¹⁴, Ar1, and Ar2 are the same as those mentioned above;
optionally, the second compound has a structure shown in Formula 2-4 or Formula 2-5;
optionally, the second compound has any one structure of Formula 2-6, Formula 2-22, Formula 2-25, Formula 2-26, Formula 2-27, Formula 2-28, Formula 2-8 to Formula 2-20.

Optionally, the second compound has a structure shown in any one of M-1 to M-723:

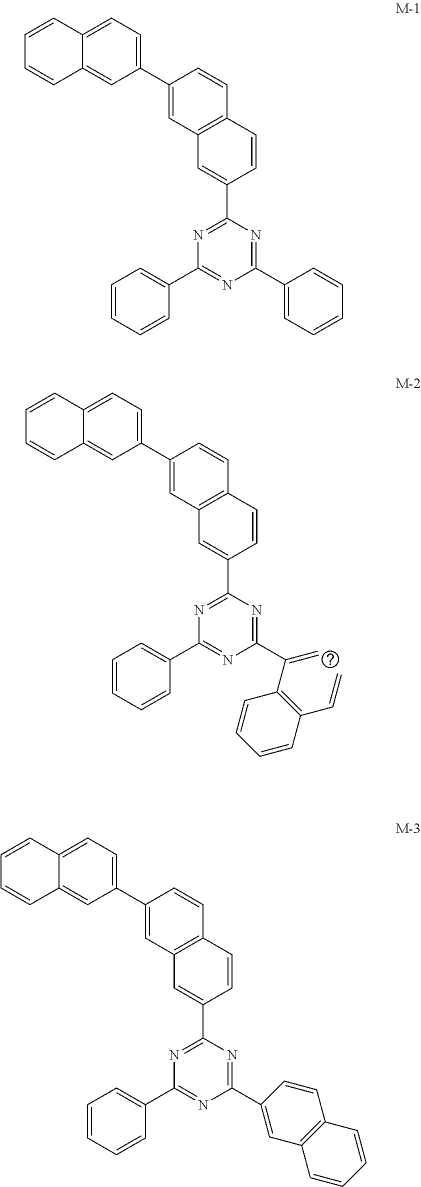 [SMILES: c1ccc(-c2nc(-c3ccccc3)nc(-c3ccc4ccc(-c5ccc6ccccc6c5)cc4c3)n2)cc1 ; C=Cc1ccccc1C(=C)c1nc(-c2ccccc2)nc(-c2ccc3ccc(-c4ccc5ccccc5c4)cc3c2)n1 ; c1ccc(-c2nc(-c3ccc4ccccc4c3)nc(-c3ccc4ccc(-c5ccc6ccccc6c5)cc4c3)n2)cc1]

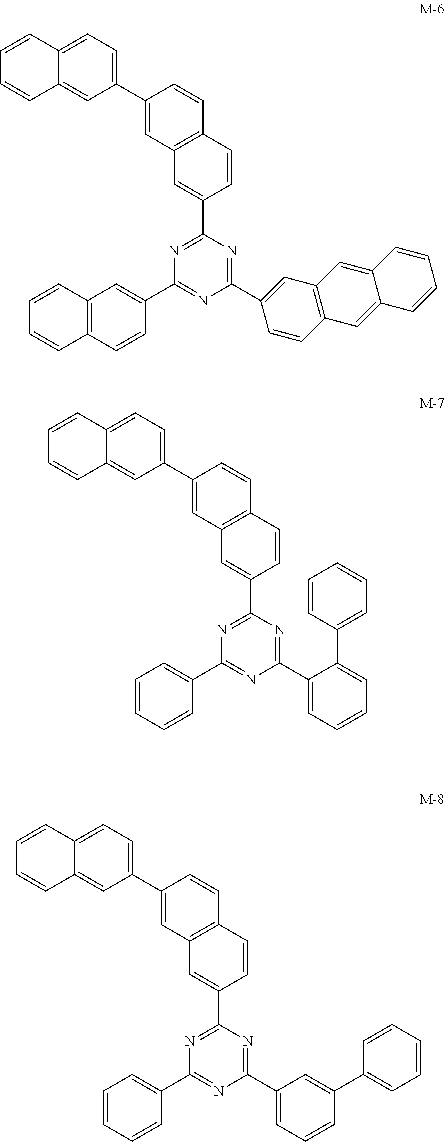 [SMILES: c1ccc2cc(-c3ccc4ccc(-c5nc(-c6ccc7ccccc7c6)nc(-c6ccc7cc8ccccc8cc7c6)n5)cc4c3)ccc2c1 ; c1ccc(-c2nc(-c3ccc4ccc(-c5ccc6ccccc6c5)cc4c3)nc(-c3ccccc3-c3ccccc3)n2)cc1 ; c1ccc(-c2cccc(-c3nc(-c4ccccc4)nc(-c4ccc5ccc(-c6ccc7ccccc7c6)cc5c4)n3)c2)cc1]

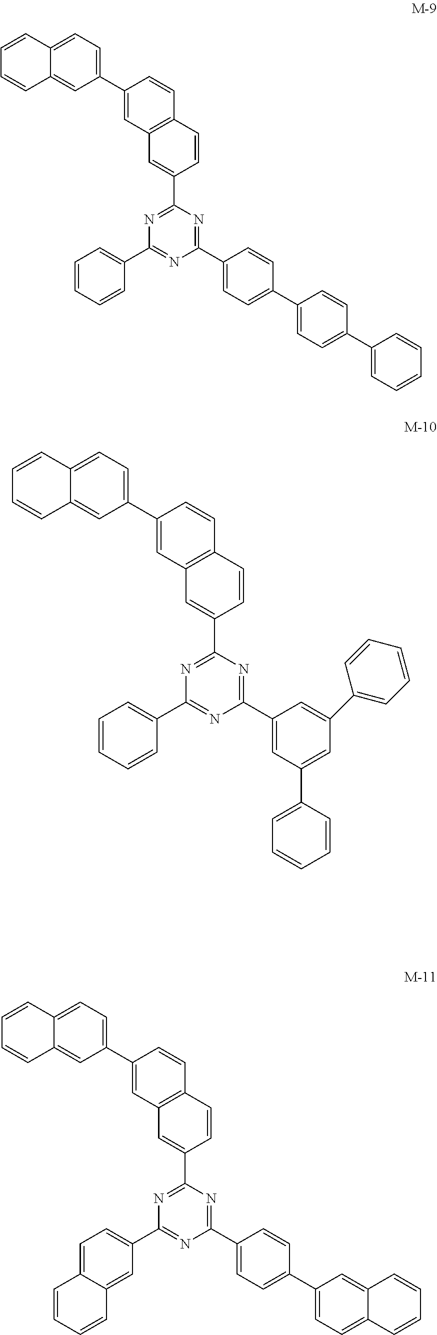 [SMILES: c1ccc(-c2ccc(-c3ccc(-c4nc(-c5ccccc5)nc(-c5ccc6ccc(-c7ccc8ccccc8c7)cc6c5)n4)cc3)cc2)cc1 ; c1ccc(-c2cc(-c3ccccc3)cc(-c3nc(-c4ccccc4)nc(-c4ccc5ccc(-c6ccc7ccccc7c6)cc5c4)n3)c2)cc1 ; c1ccc2cc(-c3ccc(-c4nc(-c5ccc6ccccc6c5)nc(-c5ccc6ccc(-c7ccc8ccccc8c7)cc6c5)n4)cc3)ccc2c1]

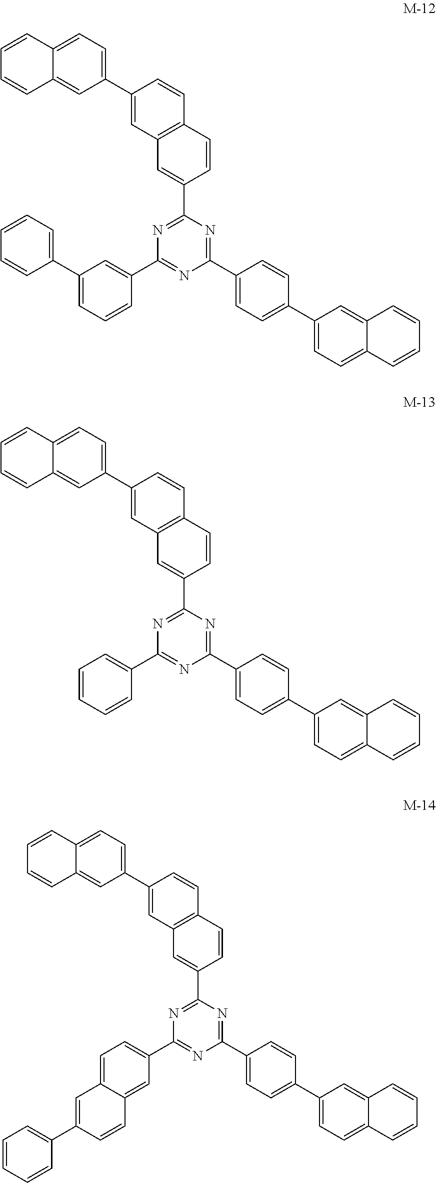 [SMILES: c1ccc(-c2cccc(-c3nc(-c4ccc(-c5ccc6ccccc6c5)cc4)nc(-c4ccc5ccc(-c6ccc7ccccc7c6)cc5c4)n3)c2)cc1 ; c1ccc(-c2nc(-c3ccc(-c4ccc5ccccc5c4)cc3)nc(-c3ccc4ccc(-c5ccc6ccccc6c5)cc4c3)n2)cc1 ; c1ccc(-c2ccc3cc(-c4nc(-c5ccc(-c6ccc7ccccc7c6)cc5)nc(-c5ccc6ccc(-c7ccc8ccccc8c7)cc6c5)n4)ccc3c2)cc1]

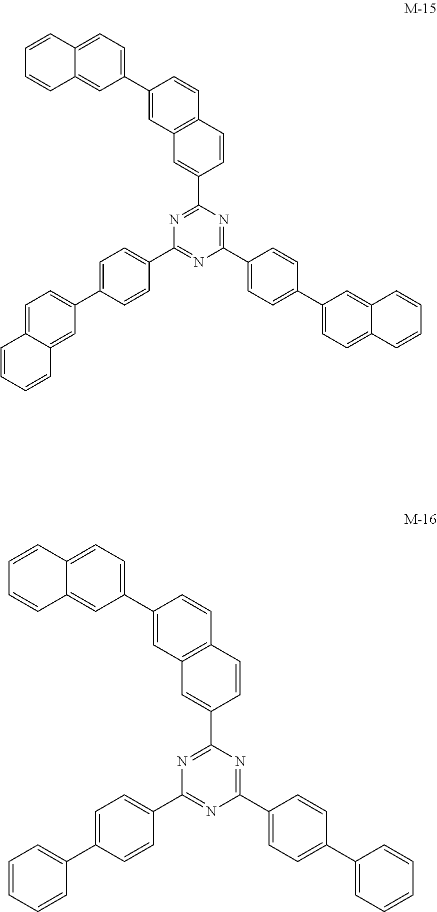 [SMILES: c1ccc2cc(-c3ccc(-c4nc(-c5ccc(-c6ccc7ccccc7c6)cc5)nc(-c5ccc6ccc(-c7ccc8ccccc8c7)cc6c5)n4)cc3)ccc2c1 ; c1ccc(-c2ccc(-c3nc(-c4ccc(-c5ccccc5)cc4)nc(-c4ccc5ccc(-c6ccc7ccccc7c6)cc5c4)n3)cc2)cc1]

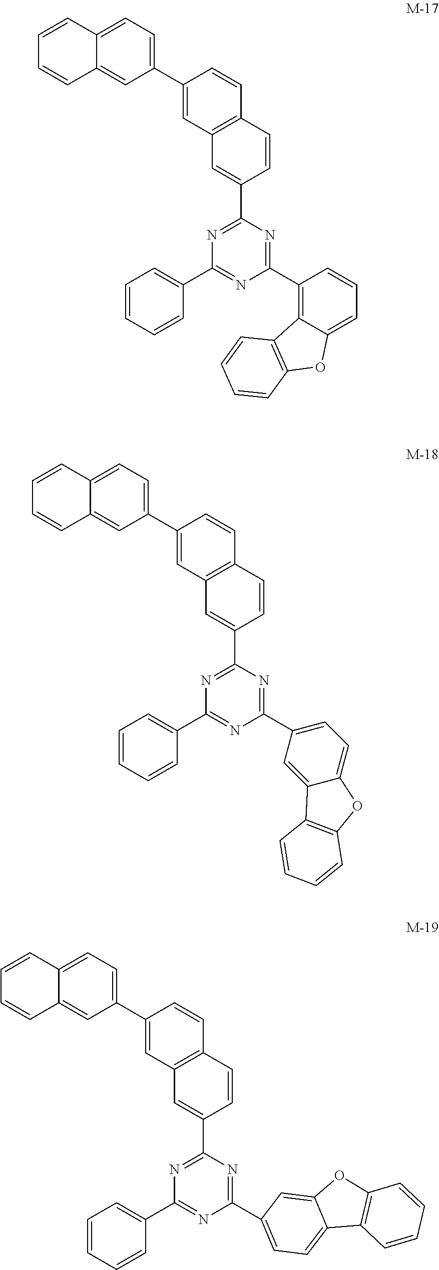 [SMILES: c1ccc(-c2nc(-c3ccc4ccc(-c5ccc6ccccc6c5)cc4c3)nc(-c3cccc4oc5ccccc5c34)n2)cc1 ; c1ccc(-c2nc(-c3ccc4ccc(-c5ccc6ccccc6c5)cc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4c(c3)oc3ccccc34)n2)cc1]

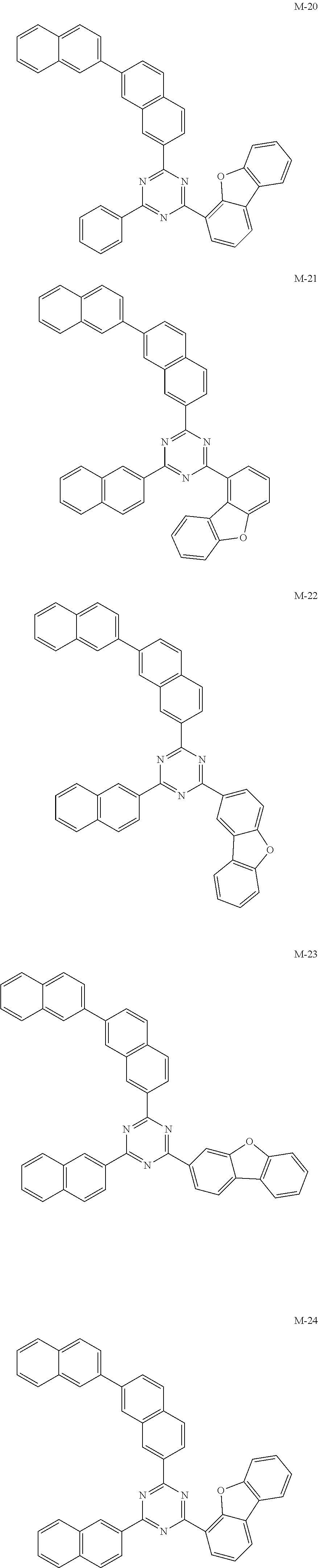

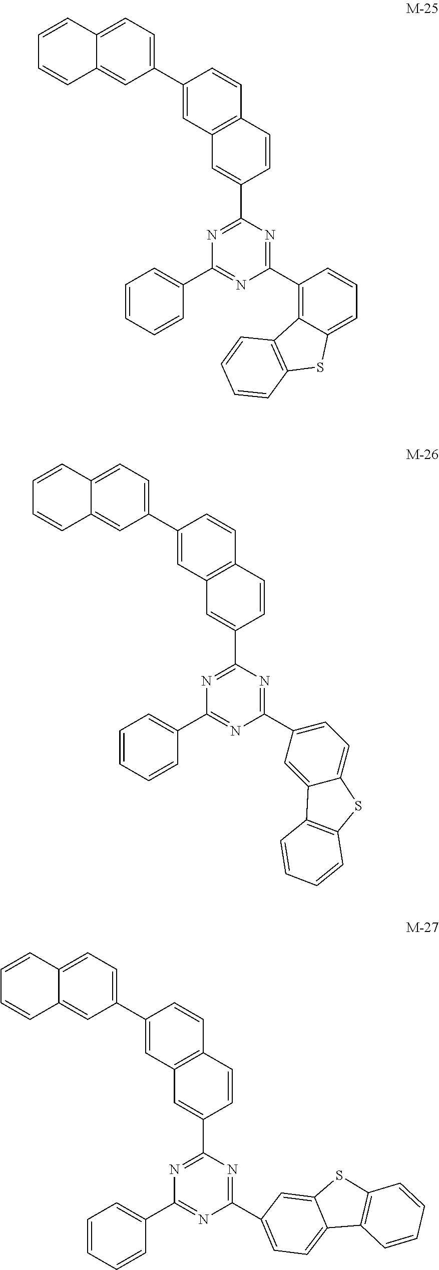 [SMILES: c1ccc(-c2nc(-c3ccc4ccc(-c5ccc6ccccc6c5)cc4c3)nc(-c3cccc4sc5ccccc5c34)n2)cc1 ; c1ccc(-c2nc(-c3ccc4ccc(-c5ccc6ccccc6c5)cc4c3)nc(-c3ccc4sc5ccccc5c4c3)n2)cc1 ; c1ccc(-c2nc(-c3ccc4ccc(-c5ccc6ccccc6c5)cc4c3)nc(-c3ccc4c(c3)sc3ccccc34)n2)cc1]

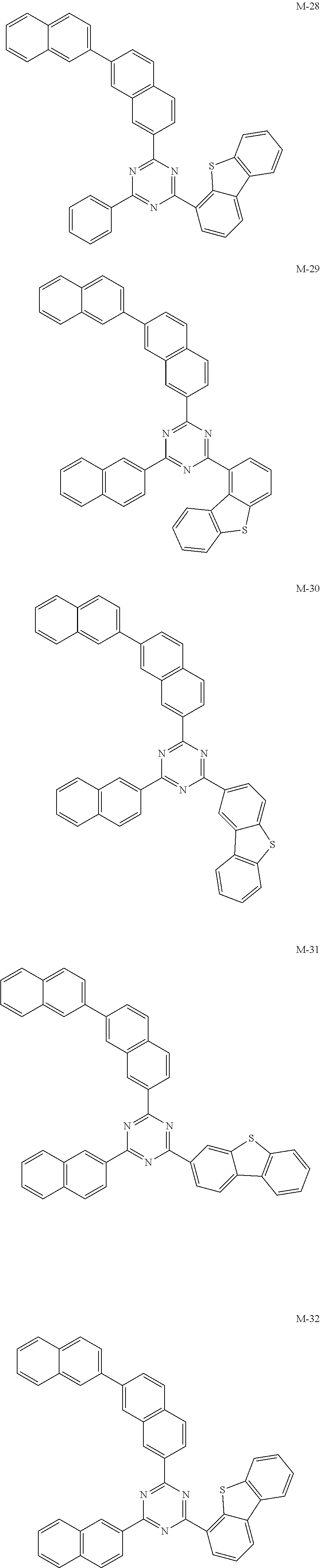

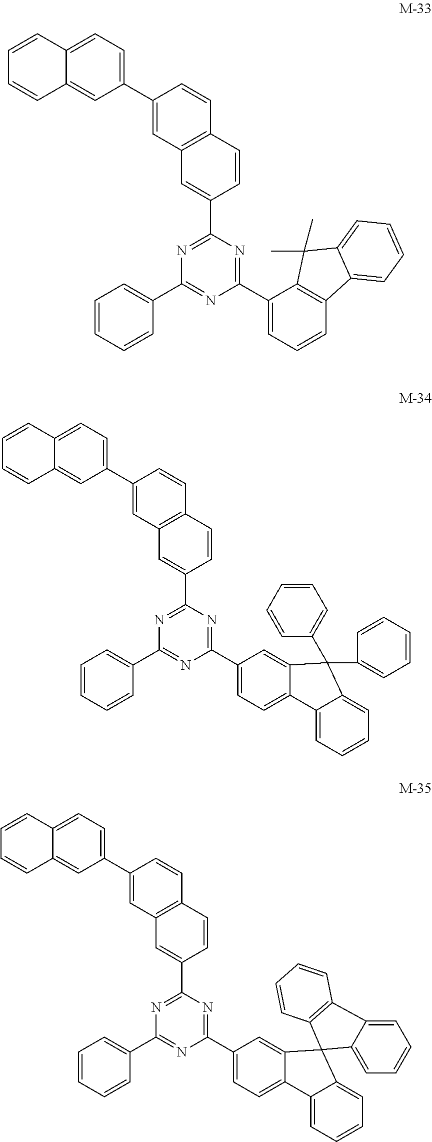 [SMILES: CC1(C)c2ccccc2-c2cccc(-c3nc(-c4ccccc4)nc(-c4ccc5ccc(-c6ccc7ccccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ccccc6c5)cc4c3)n2)cc1 ; c1ccc(-c2nc(-c3ccc4c(c3)C3(c5ccccc5-c5ccccc53)c3ccccc3-4)nc(-c3ccc4ccc(-c5ccc6ccccc6c5)cc4c3)n2)cc1]

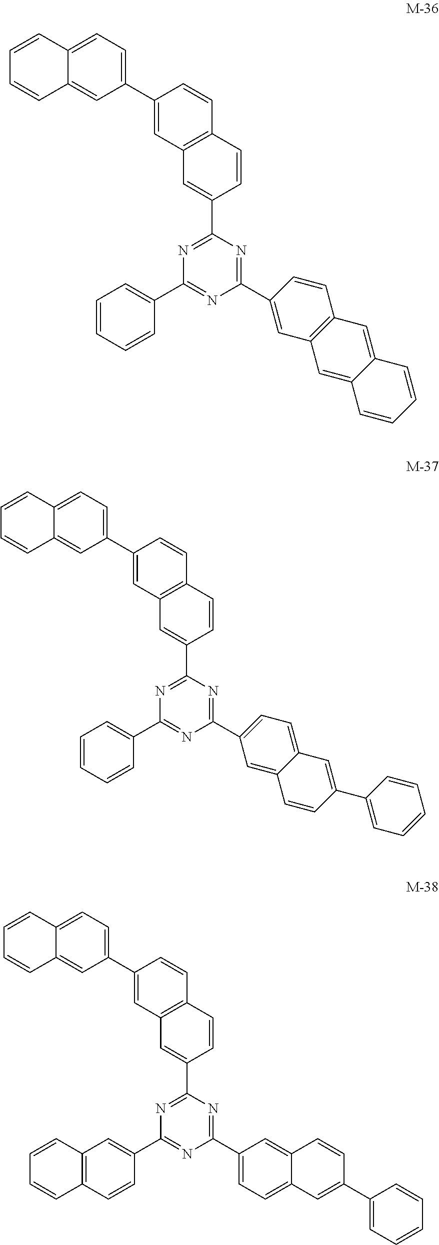 [SMILES: c1ccc(-c2nc(-c3ccc4ccc(-c5ccc6ccccc6c5)cc4c3)nc(-c3ccc4cc5ccccc5cc4c3)n2)cc1 ; c1ccc(-c2ccc3cc(-c4nc(-c5ccccc5)nc(-c5ccc6ccc(-c7ccc8ccccc8c7)cc6c5)n4)ccc3c2)cc1 ; c1ccc(-c2ccc3cc(-c4nc(-c5ccc6ccccc6c5)nc(-c5ccc6ccc(-c7ccc8ccccc8c7)cc6c5)n4)ccc3c2)cc1]

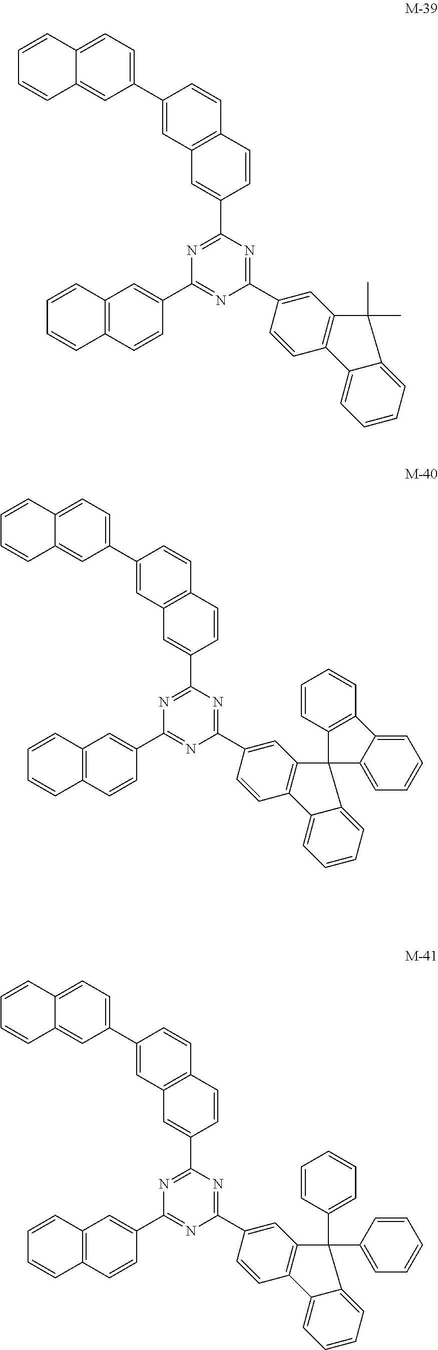 [SMILES: CC1(C)c2ccccc2-c2ccc(-c3nc(-c4ccc5ccccc5c4)nc(-c4ccc5ccc(-c6ccc7ccccc7c6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6ccc7ccccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ccccc8c7)cc6c5)n4)cc32)cc1]

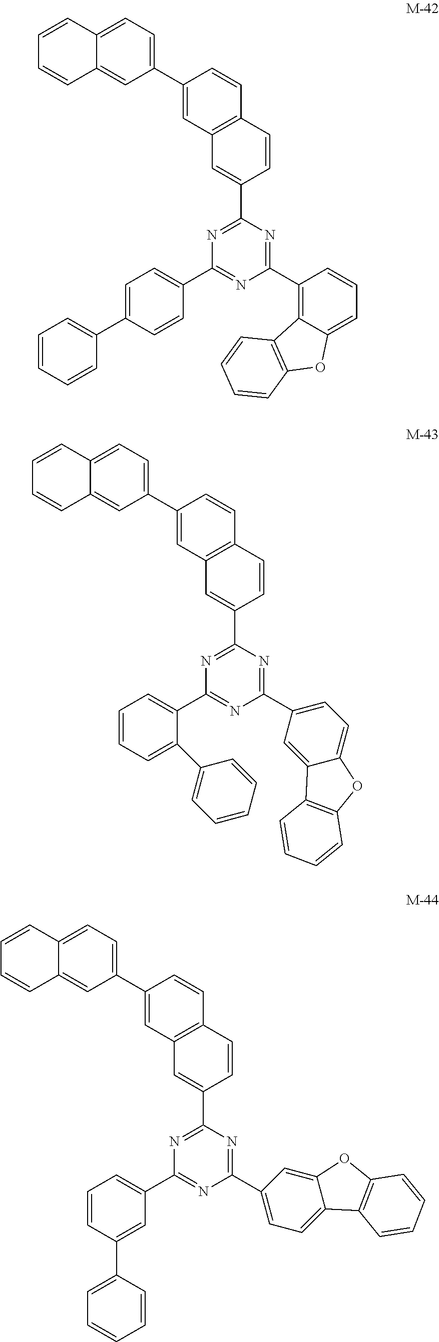 [SMILES: c1ccc(-c2ccc(-c3nc(-c4ccc5ccc(-c6ccc7ccccc7c6)cc5c4)nc(-c4cccc5oc6ccccc6c45)n3)cc2)cc1 ; c1ccc(-c2ccccc2-c2nc(-c3ccc4ccc(-c5ccc6ccccc6c5)cc4c3)nc(-c3ccc4oc5ccccc5c4c3)n2)cc1 ; c1ccc(-c2cccc(-c3nc(-c4ccc5ccc(-c6ccc7ccccc7c6)cc5c4)nc(-c4ccc5c(c4)oc4ccccc45)n3)c2)cc1]

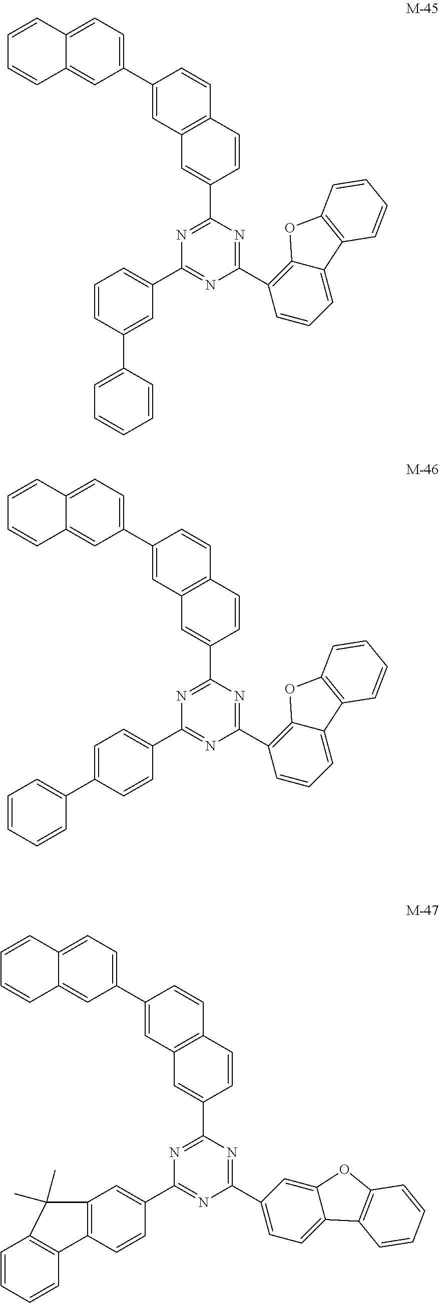 [SMILES: c1ccc(-c2cccc(-c3nc(-c4ccc5ccc(-c6ccc7ccccc7c6)cc5c4)nc(-c4cccc5c4oc4ccccc45)n3)c2)cc1 ; c1ccc(-c2ccc(-c3nc(-c4ccc5ccc(-c6ccc7ccccc7c6)cc5c4)nc(-c4cccc5c4oc4ccccc45)n3)cc2)cc1 ; CC1(C)c2ccccc2-c2ccc(-c3nc(-c4ccc5ccc(-c6ccc7ccccc7c6)cc5c4)nc(-c4ccc5c(c4)oc4ccccc45)n3)cc21]

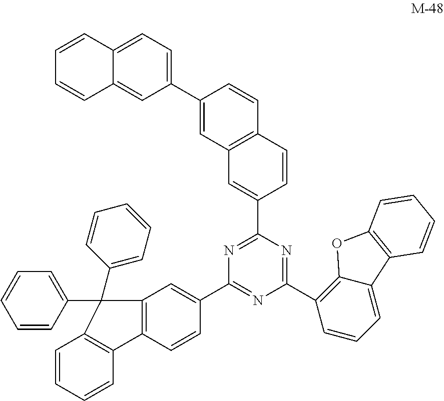 [SMILES: c1ccc(C2(c3ccccc3)c3ccccc3-c3ccc(-c4nc(-c5ccc6ccc(-c7ccc8ccccc8c7)cc6c5)nc(-c5cccc6c5oc5ccccc56)n4)cc32)cc1]

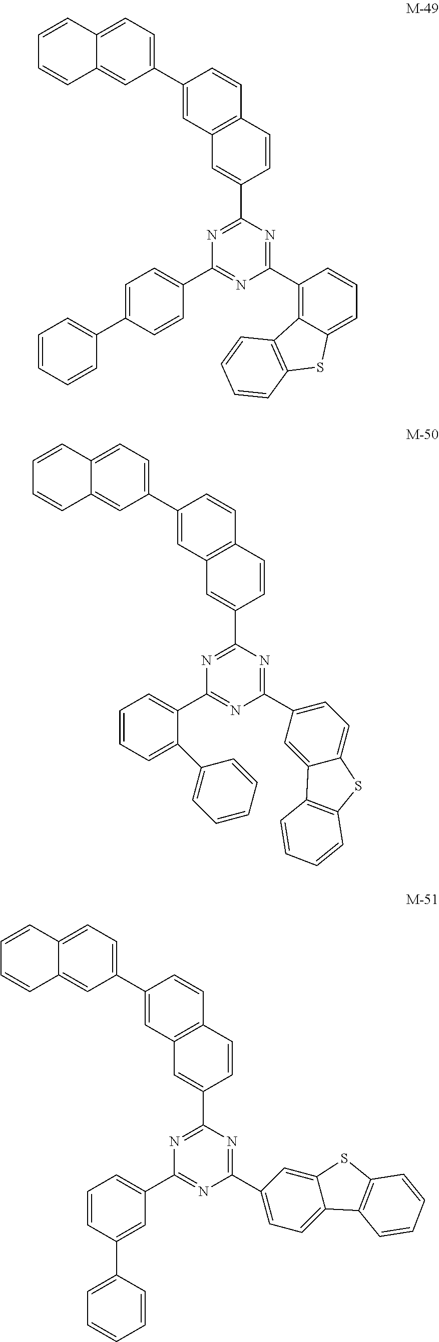 [SMILES: c1ccc(-c2ccc(-c3nc(-c4ccc5ccc(-c6ccc7ccccc7c6)cc5c4)nc(-c4cccc5sc6ccccc6c45)n3)cc2)cc1 ; c1ccc(-c2ccccc2-c2nc(-c3ccc4ccc(-c5ccc6ccccc6c5)cc4c3)nc(-c3ccc4sc5ccccc5c4c3)n2)cc1 ; c1ccc(-c2cccc(-c3nc(-c4ccc5ccc(-c6ccc7ccccc7c6)cc5c4)nc(-c4ccc5c(c4)sc4ccccc45)n3)c2)cc1]

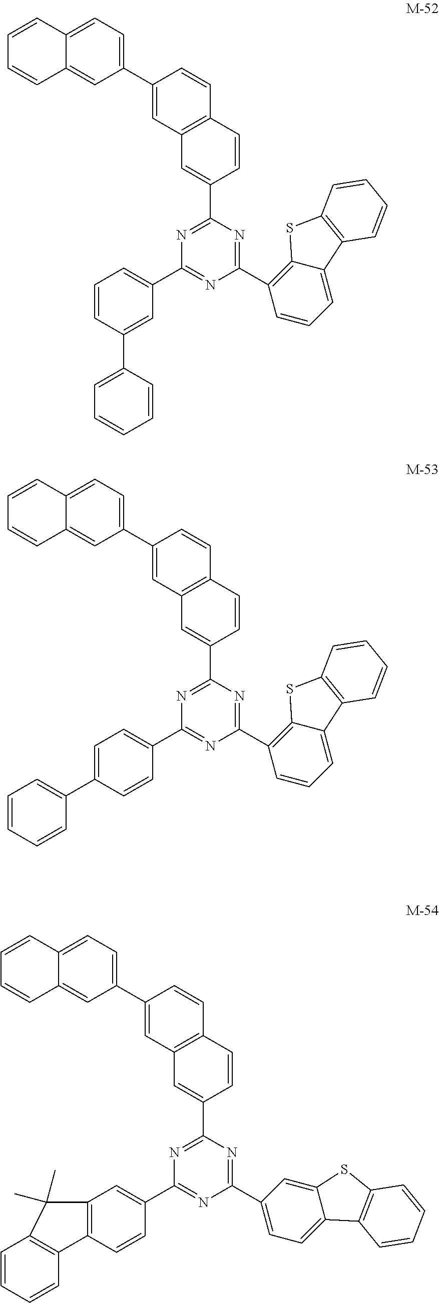 [SMILES: c1ccc(-c2cccc(-c3nc(-c4ccc5ccc(-c6ccc7ccccc7c6)cc5c4)nc(-c4cccc5c4sc4ccccc45)n3)c2)cc1 ; c1ccc(-c2ccc(-c3nc(-c4ccc5ccc(-c6ccc7ccccc7c6)cc5c4)nc(-c4cccc5c4sc4ccccc45)n3)cc2)cc1 ; CC1(C)c2ccccc2-c2ccc(-c3nc(-c4ccc5ccc(-c6ccc7ccccc7c6)cc5c4)nc(-c4ccc5c(c4)sc4ccccc45)n3)cc21]

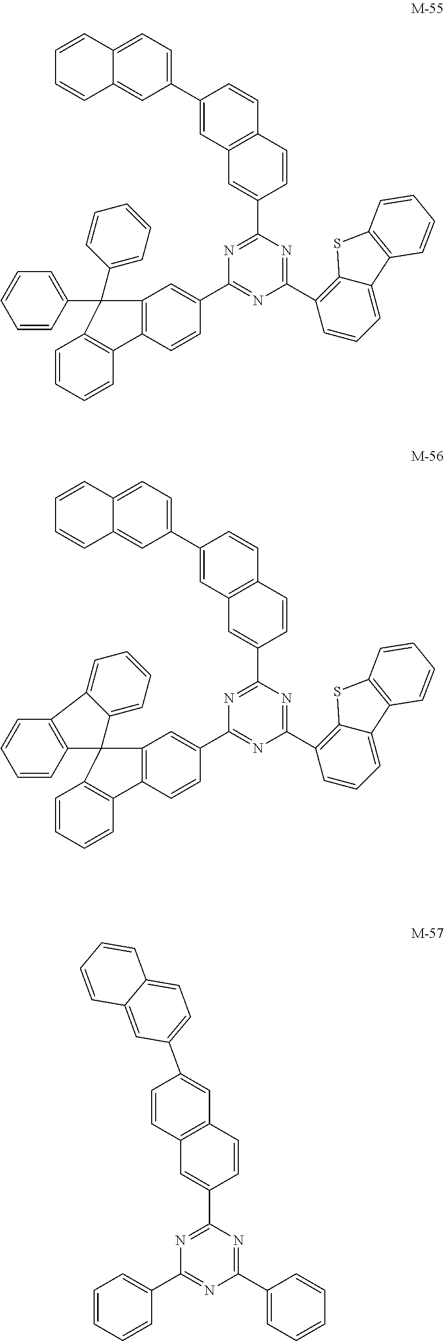 [SMILES: c1ccc(C2(c3ccccc3)c3ccccc3-c3ccc(-c4nc(-c5ccc6ccc(-c7ccc8ccccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6ccc7ccccc7c6)cc5c4)nc(-c4cccc5c4sc4ccccc45)n3)cc21 ; c1ccc(-c2nc(-c3ccccc3)nc(-c3ccc4cc(-c5ccc6ccccc6c5)ccc4c3)n2)cc1]

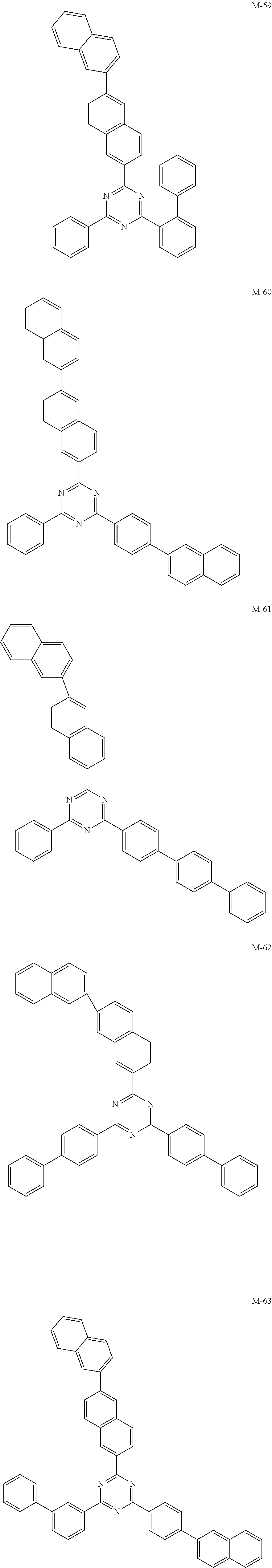 [SMILES: c1ccc(-c2nc(-c3ccc4cc(-c5ccc6ccccc6c5)ccc4c3)nc(-c3ccccc3-c3ccccc3)n2)cc1 ; c1ccc(-c2nc(-c3ccc(-c4ccc5ccccc5c4)cc3)nc(-c3ccc4cc(-c5ccc6ccccc6c5)ccc4c3)n2)cc1 ; c1ccc(-c2ccc(-c3ccc(-c4nc(-c5ccccc5)nc(-c5ccc6cc(-c7ccc8ccccc8c7)ccc6c5)n4)cc3)cc2)cc1 ; c1ccc(-c2ccc(-c3nc(-c4ccc(-c5ccccc5)cc4)nc(-c4ccc5ccc(-c6ccc7ccccc7c6)cc5c4)n3)cc2)cc1 ; c1ccc(-c2cccc(-c3nc(-c4ccc(-c5ccc6ccccc6c5)cc4)nc(-c4ccc5cc(-c6ccc7ccccc7c6)ccc5c4)n3)c2)cc1]

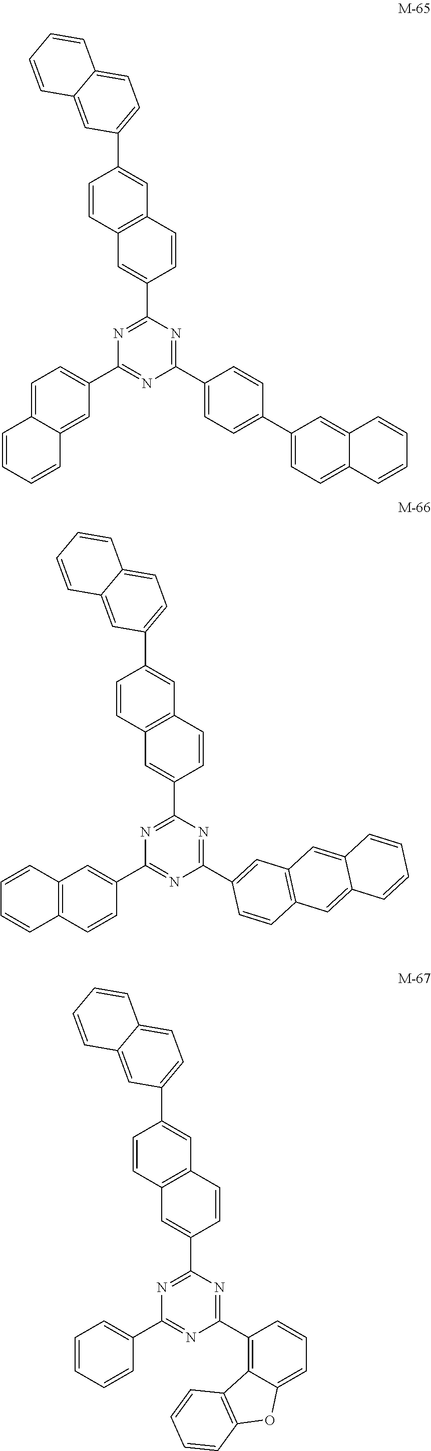 [SMILES: c1ccc2cc(-c3ccc(-c4nc(-c5ccc6ccccc6c5)nc(-c5ccc6cc(-c7ccc8ccccc8c7)ccc6c5)n4)cc3)ccc2c1 ; c1ccc2cc(-c3ccc4cc(-c5nc(-c6ccc7ccccc7c6)nc(-c6ccc7cc8ccccc8cc7c6)n5)ccc4c3)ccc2c1 ; c1ccc(-c2nc(-c3ccc4cc(-c5ccc6ccccc6c5)ccc4c3)nc(-c3cccc4oc5ccccc5c34)n2)cc1]

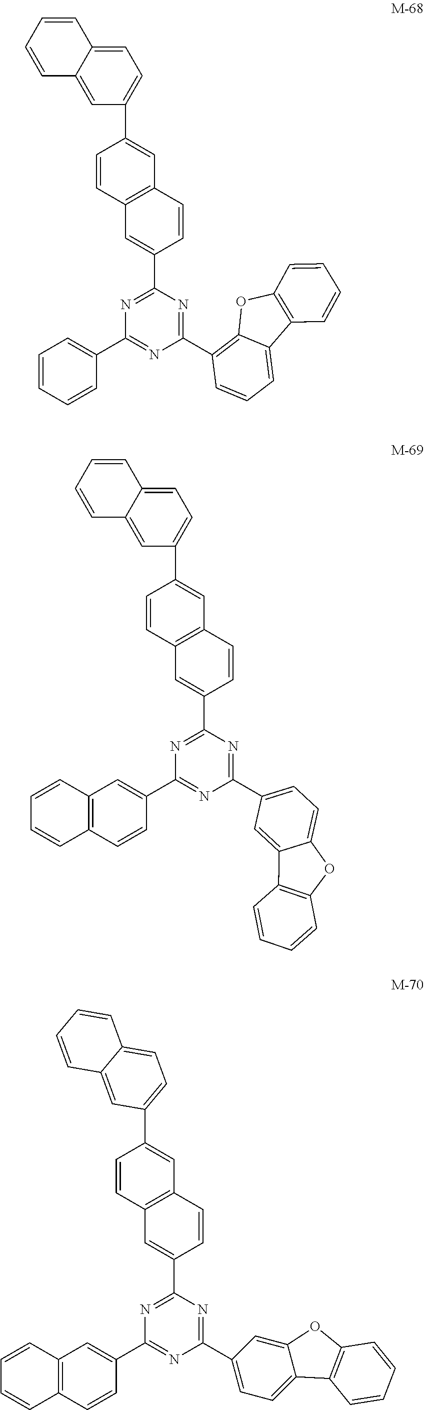 [SMILES: c1ccc(-c2nc(-c3ccc4cc(-c5ccc6ccccc6c5)ccc4c3)nc(-c3cccc4c3oc3ccccc34)n2)cc1 ; c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3ccc4cc(-c5nc(-c6ccc7ccccc7c6)nc(-c6ccc7c(c6)oc6ccccc67)n5)ccc4c3)ccc2c1]

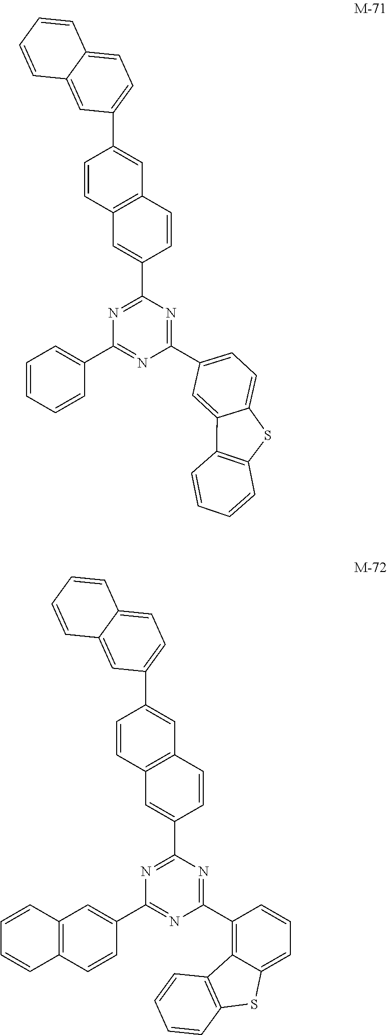 [SMILES: c1ccc(-c2nc(-c3ccc4cc(-c5ccc6ccccc6c5)ccc4c3)nc(-c3ccc4sc5ccccc5c4c3)n2)cc1 ; c1ccc2cc(-c3ccc4cc(-c5nc(-c6ccc7ccccc7c6)nc(-c6cccc7sc8ccccc8c67)n5)ccc4c3)ccc2c1]

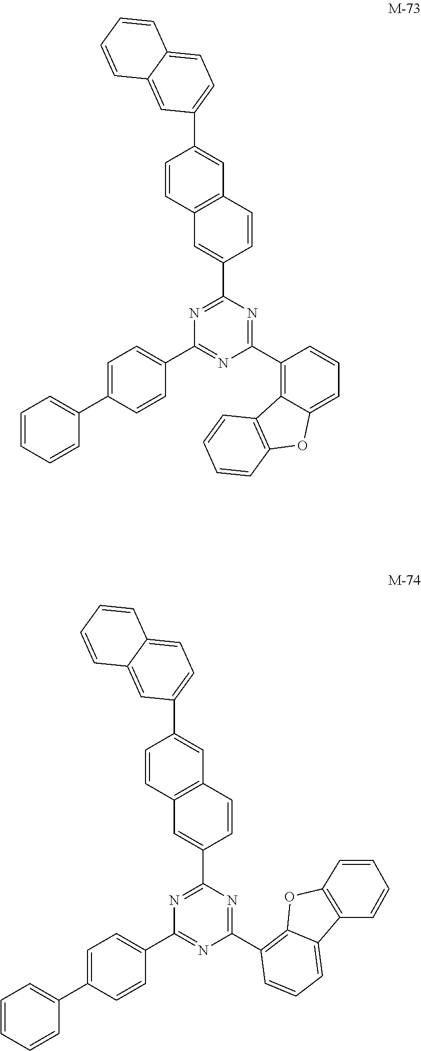 [SMILES: c1ccc(-c2ccc(-c3nc(-c4ccc5cc(-c6ccc7ccccc7c6)ccc5c4)nc(-c4cccc5oc6ccccc6c45)n3)cc2)cc1 ; c1ccc(-c2ccc(-c3nc(-c4ccc5cc(-c6ccc7ccccc7c6)ccc5c4)nc(-c4cccc5c4oc4ccccc45)n3)cc2)cc1]

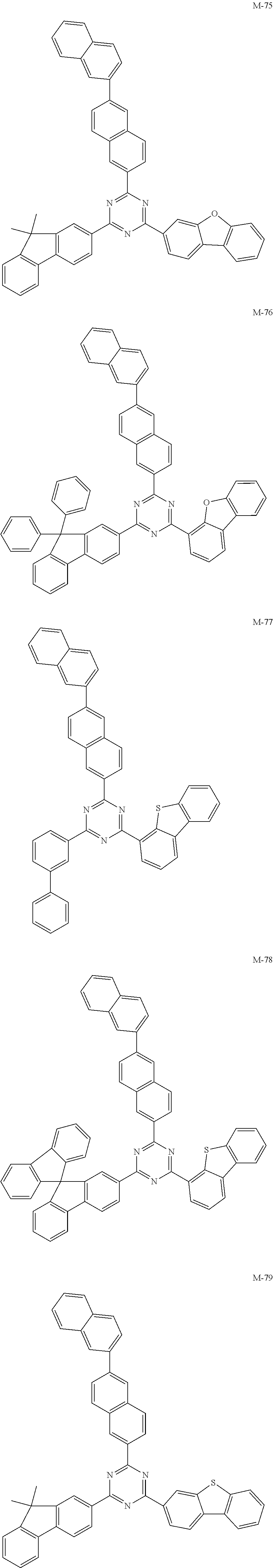 [SMILES: CC1(C)c2ccccc2-c2ccc(-c3nc(-c4ccc5cc(-c6ccc7ccccc7c6)ccc5c4)nc(-c4ccc5c(c4)oc4ccccc45)n3)cc21 ; c1ccc(C2(c3ccccc3)c3ccccc3-c3ccc(-c4nc(-c5ccc6cc(-c7ccc8ccccc8c7)ccc6c5)nc(-c5cccc6c5oc5ccccc56)n4)cc32)cc1 ; c1ccc(-c2cccc(-c3nc(-c4ccc5cc(-c6ccc7ccccc7c6)ccc5c4)nc(-c4cccc5c4sc4ccccc45)n3)c2)cc1 ; c1ccc2c(c1)-c1ccccc1C21c2ccccc2-c2ccc(-c3nc(-c4ccc5cc(-c6ccc7ccccc7c6)ccc5c4)nc(-c4cccc5c4sc4ccccc45)n3)cc21 ; CC1(C)c2ccccc2-c2ccc(-c3nc(-c4ccc5cc(-c6ccc7ccccc7c6)ccc5c4)nc(-c4ccc5c(c4)sc4ccccc45)n3)cc21]

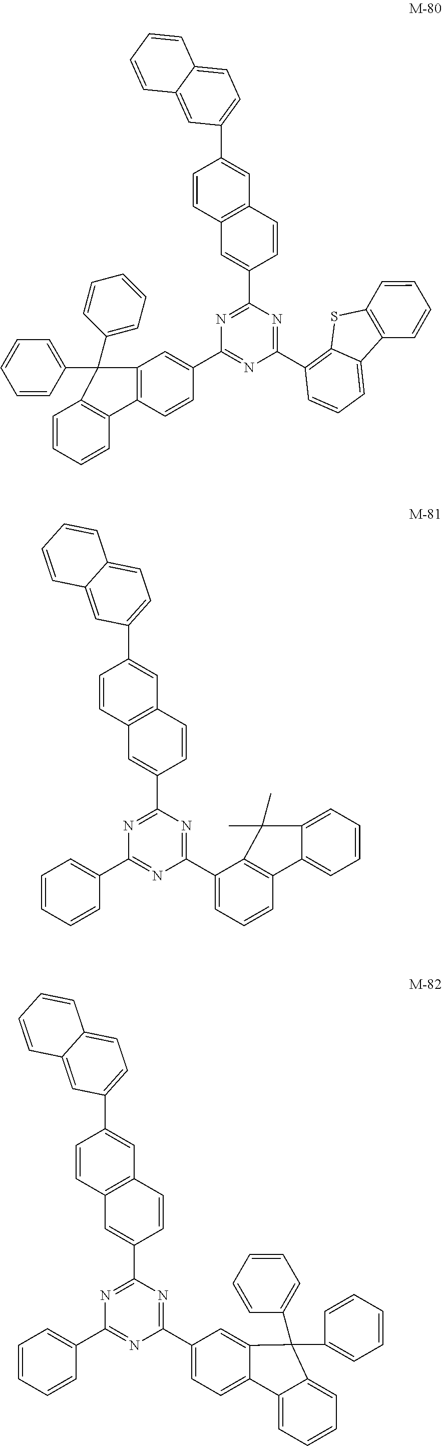 [SMILES: c1ccc(C2(c3ccccc3)c3ccccc3-c3ccc(-c4nc(-c5ccc6cc(-c7ccc8ccccc8c7)ccc6c5)nc(-c5cccc6c5sc5ccccc56)n4)cc32)cc1 ; CC1(C)c2ccccc2-c2cccc(-c3nc(-c4ccccc4)nc(-c4ccc5cc(-c6ccc7ccccc7c6)ccc5c4)n3)c21 ; c1ccc(-c2nc(-c3ccc4c(c3)C(c3ccccc3)(c3ccccc3)c3ccccc3-4)nc(-c3ccc4cc(-c5ccc6ccccc6c5)ccc4c3)n2)cc1]

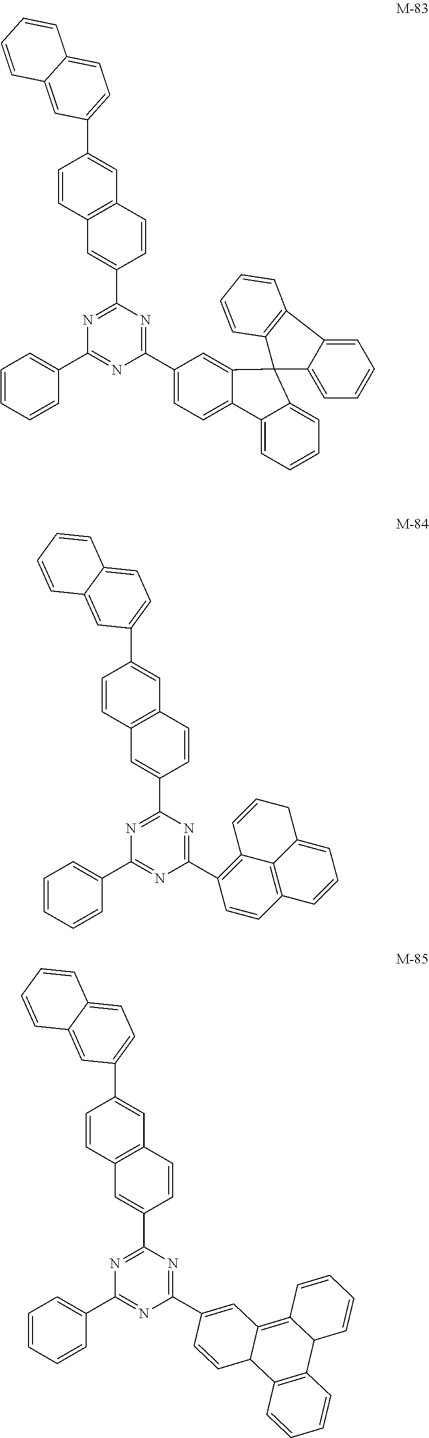

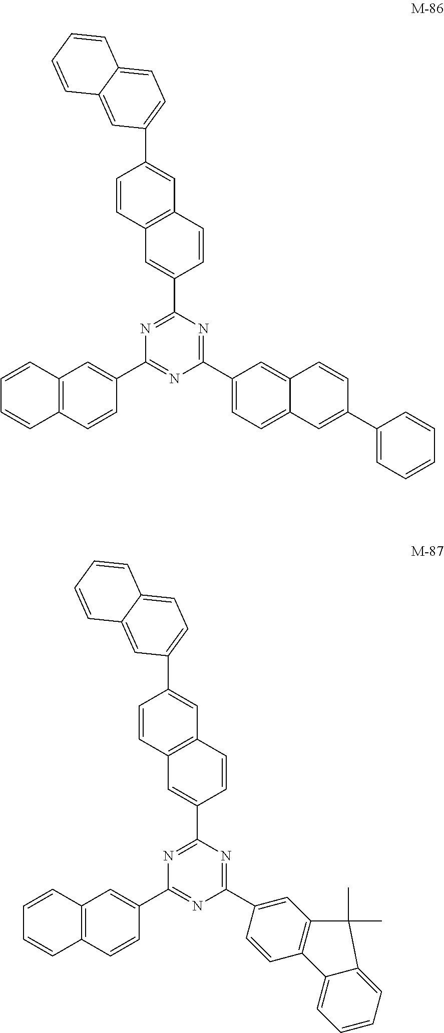 [SMILES: c1ccc(-c2ccc3cc(-c4nc(-c5ccc6ccccc6c5)nc(-c5ccc6cc(-c7ccc8ccccc8c7)ccc6c5)n4)ccc3c2)cc1 ; CC1(C)c2ccccc2-c2ccc(-c3nc(-c4ccc5ccccc5c4)nc(-c4ccc5cc(-c6ccc7ccccc7c6)ccc5c4)n3)cc21]

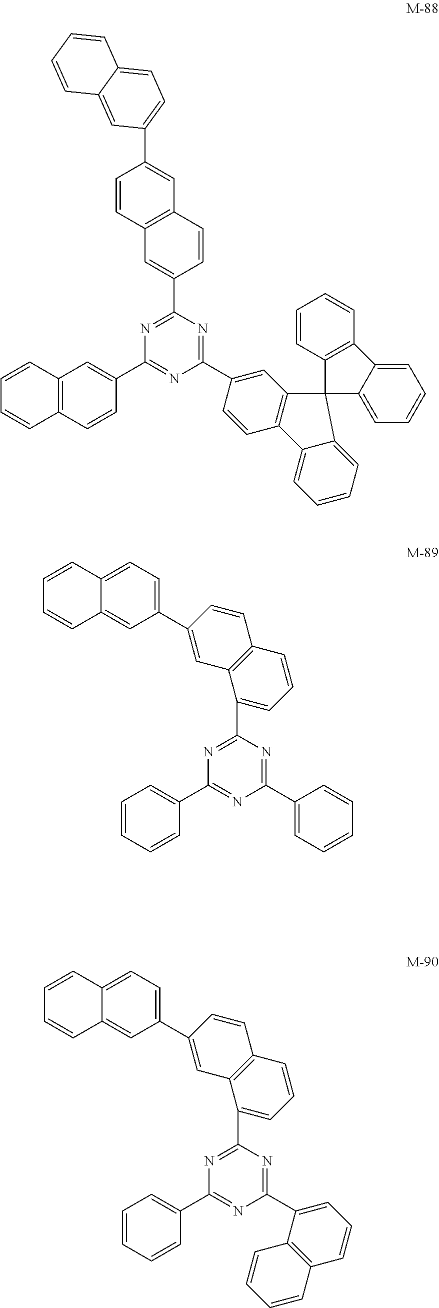 [SMILES: c1ccc2c(c1)-c1ccccc1C21c2ccccc2-c2ccc(-c3nc(-c4ccc5ccccc5c4)nc(-c4ccc5cc(-c6ccc7ccccc7c6)ccc5c4)n3)cc21 ; c1ccc(-c2nc(-c3ccccc3)nc(-c3cccc4ccc(-c5ccc6ccccc6c5)cc34)n2)cc1 ; c1ccc(-c2nc(-c3cccc4ccccc34)nc(-c3cccc4ccc(-c5ccc6ccccc6c5)cc34)n2)cc1]

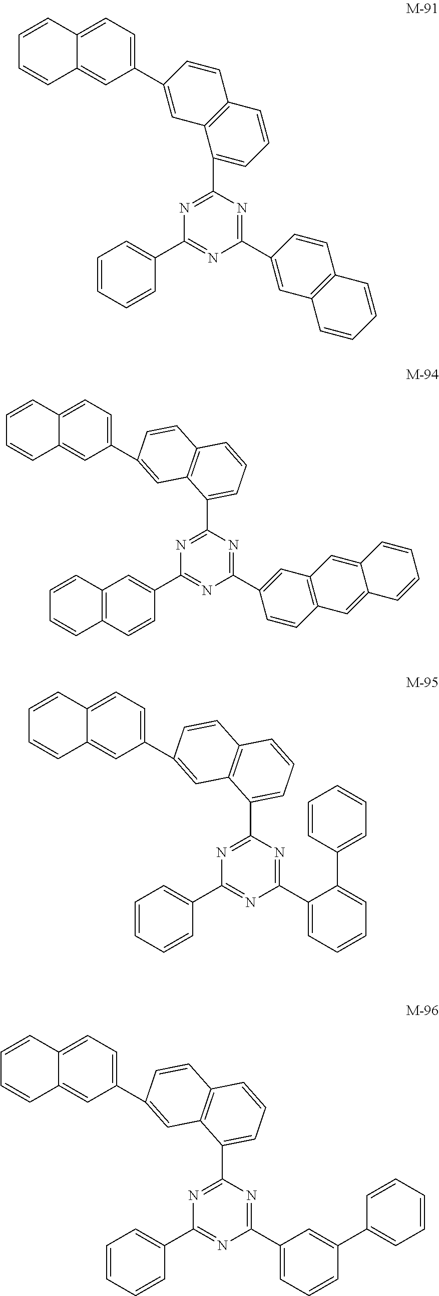 [SMILES: c1ccc(-c2nc(-c3ccc4ccccc4c3)nc(-c3cccc4ccc(-c5ccc6ccccc6c5)cc34)n2)cc1 ; c1ccc2cc(-c3ccc4cccc(-c5nc(-c6ccc7ccccc7c6)nc(-c6ccc7cc8ccccc8cc7c6)n5)c4c3)ccc2c1 ; c1ccc(-c2nc(-c3ccccc3-c3ccccc3)nc(-c3cccc4ccc(-c5ccc6ccccc6c5)cc34)n2)cc1 ; c1ccc(-c2cccc(-c3nc(-c4ccccc4)nc(-c4cccc5ccc(-c6ccc7ccccc7c6)cc45)n3)c2)cc1]

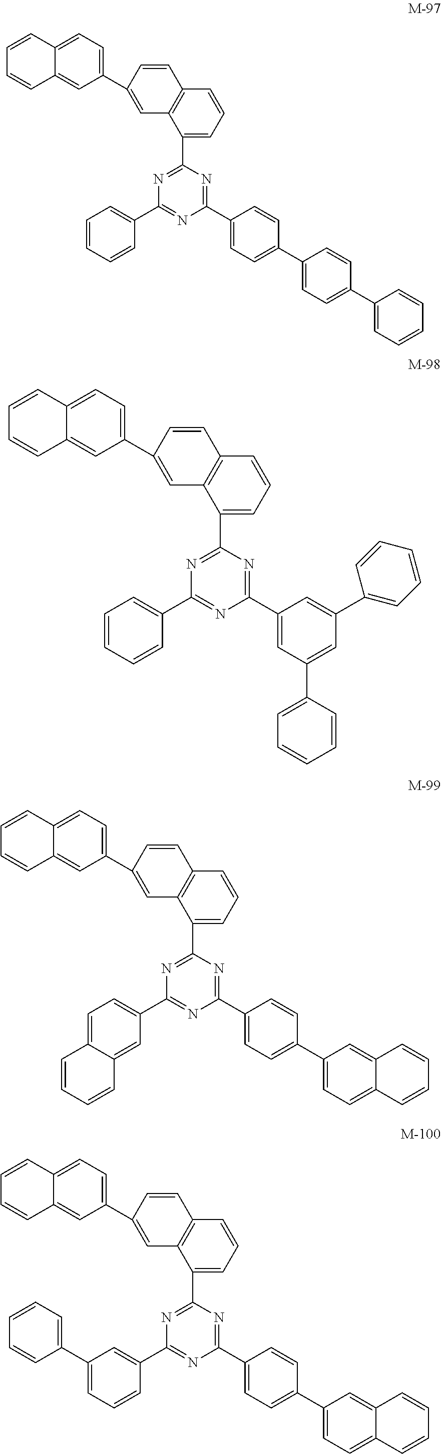 [SMILES: c1ccc(-c2ccc(-c3ccc(-c4nc(-c5ccccc5)nc(-c5cccc6ccc(-c7ccc8ccccc8c7)cc56)n4)cc3)cc2)cc1 ; c1ccc(-c2cc(-c3ccccc3)cc(-c3nc(-c4ccccc4)nc(-c4cccc5ccc(-c6ccc7ccccc7c6)cc45)n3)c2)cc1 ; c1ccc2cc(-c3ccc(-c4nc(-c5ccc6ccccc6c5)nc(-c5cccc6ccc(-c7ccc8ccccc8c7)cc56)n4)cc3)ccc2c1 ; c1ccc(-c2cccc(-c3nc(-c4ccc(-c5ccc6ccccc6c5)cc4)nc(-c4cccc5ccc(-c6ccc7ccccc7c6)cc45)n3)c2)cc1]

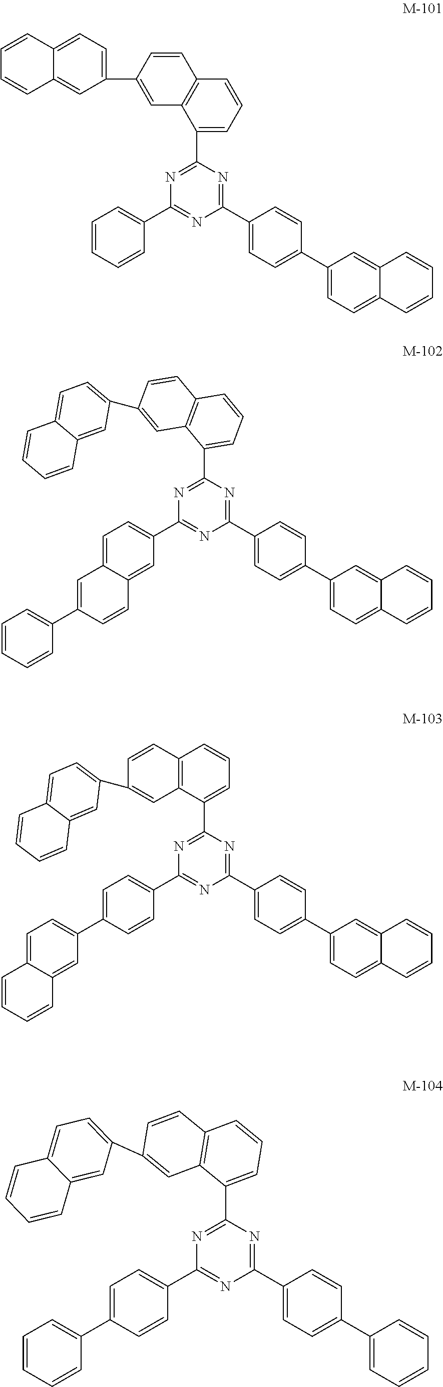 [SMILES: c1ccc(-c2nc(-c3ccc(-c4ccc5ccccc5c4)cc3)nc(-c3cccc4ccc(-c5ccc6ccccc6c5)cc34)n2)cc1 ; c1ccc(-c2ccc3cc(-c4nc(-c5ccc(-c6ccc7ccccc7c6)cc5)nc(-c5cccc6ccc(-c7ccc8ccccc8c7)cc56)n4)ccc3c2)cc1 ; c1ccc2cc(-c3ccc(-c4nc(-c5ccc(-c6ccc7ccccc7c6)cc5)nc(-c5cccc6ccc(-c7ccc8ccccc8c7)cc56)n4)cc3)ccc2c1 ; c1ccc(-c2ccc(-c3nc(-c4ccc(-c5ccccc5)cc4)nc(-c4cccc5ccc(-c6ccc7ccccc7c6)cc45)n3)cc2)cc1]

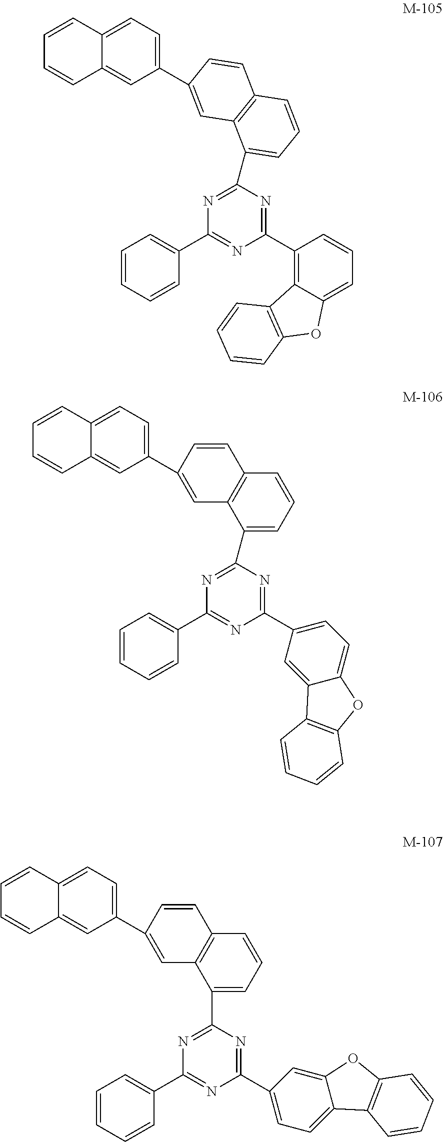 [SMILES: c1ccc(-c2nc(-c3cccc4ccc(-c5ccc6ccccc6c5)cc34)nc(-c3cccc4oc5ccccc5c34)n2)cc1 ; c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4c(c3)oc3ccccc34)nc(-c3cccc4ccc(-c5ccc6ccccc6c5)cc34)n2)cc1]

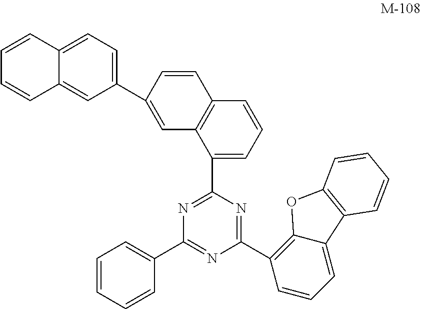 [SMILES: c1ccc(-c2nc(-c3cccc4ccc(-c5ccc6ccccc6c5)cc34)nc(-c3cccc4c3oc3ccccc34)n2)cc1]

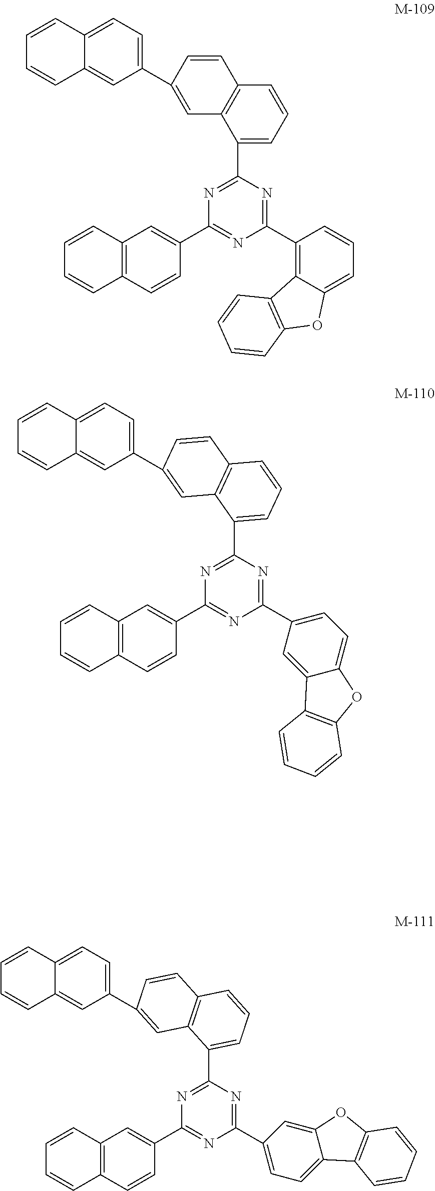 [SMILES: c1ccc2cc(-c3ccc4cccc(-c5nc(-c6ccc7ccccc7c6)nc(-c6cccc7oc8ccccc8c67)n5)c4c3)ccc2c1 ; c1ccc2cc(-c3ccc4cccc(-c5nc(-c6ccc7ccccc7c6)nc(-c6ccc7oc8ccccc8c7c6)n5)c4c3)ccc2c1 ; c1ccc2cc(-c3ccc4cccc(-c5nc(-c6ccc7ccccc7c6)nc(-c6ccc7c(c6)oc6ccccc67)n5)c4c3)ccc2c1]

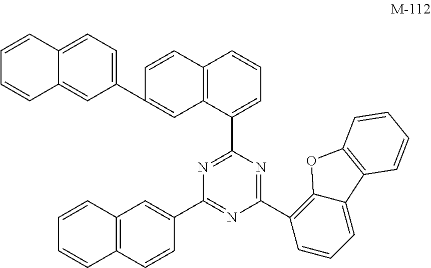 [SMILES: c1ccc2cc(-c3ccc4cccc(-c5nc(-c6ccc7ccccc7c6)nc(-c6cccc7c6oc6ccccc67)n5)c4c3)ccc2c1]

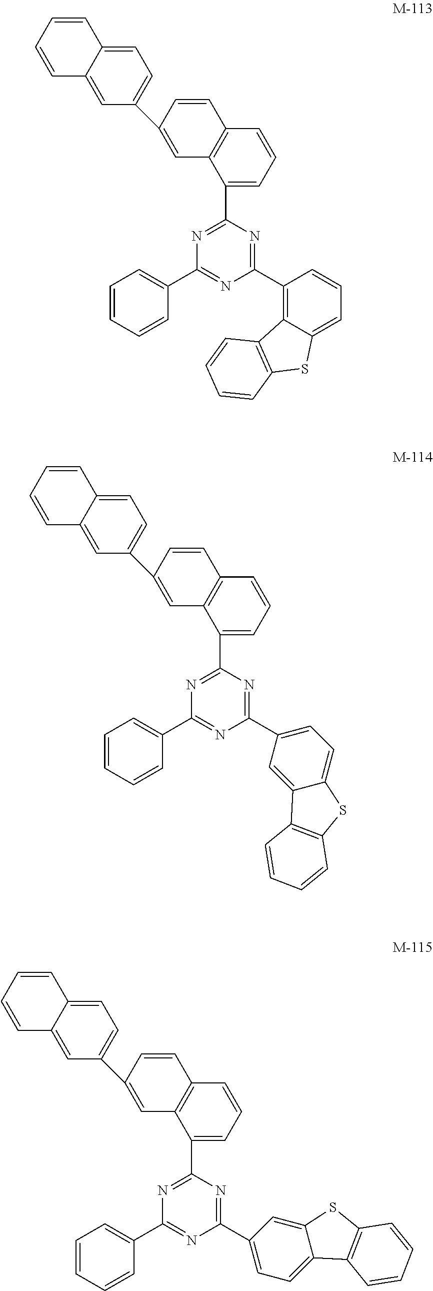 [SMILES: c1ccc(-c2nc(-c3cccc4ccc(-c5ccc6ccccc6c5)cc34)nc(-c3cccc4sc5ccccc5c34)n2)cc1 ; c1ccc(-c2nc(-c3ccc4sc5ccccc5c4c3)nc(-c3cccc4ccc(-c5ccc6ccccc6c5)cc34)n2)cc1 ; c1ccc(-c2nc(-c3ccc4c(c3)sc3ccccc34)nc(-c3cccc4ccc(-c5ccc6ccccc6c5)cc34)n2)cc1]

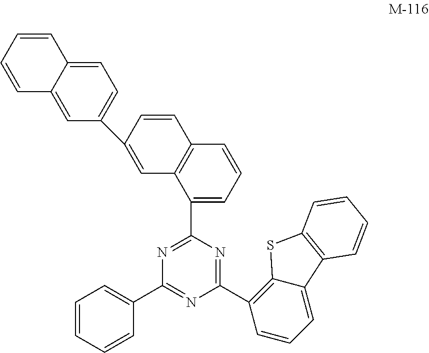 [SMILES: c1ccc(-c2nc(-c3cccc4ccc(-c5ccc6ccccc6c5)cc34)nc(-c3cccc4c3sc3ccccc34)n2)cc1]

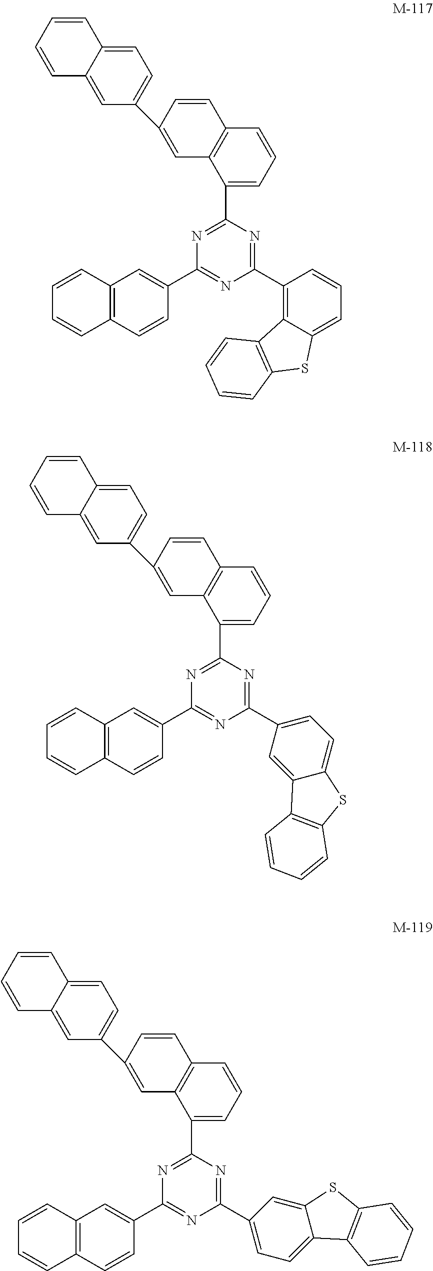 [SMILES: c1ccc2cc(-c3ccc4cccc(-c5nc(-c6ccc7ccccc7c6)nc(-c6cccc7sc8ccccc8c67)n5)c4c3)ccc2c1 ; c1ccc2cc(-c3ccc4cccc(-c5nc(-c6ccc7ccccc7c6)nc(-c6ccc7sc8ccccc8c7c6)n5)c4c3)ccc2c1 ; c1ccc2cc(-c3ccc4cccc(-c5nc(-c6ccc7ccccc7c6)nc(-c6ccc7c(c6)sc6ccccc67)n5)c4c3)ccc2c1]

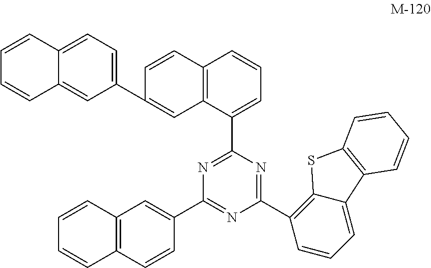 [SMILES: c1ccc2cc(-c3ccc4cccc(-c5nc(-c6ccc7ccccc7c6)nc(-c6cccc7c6sc6ccccc67)n5)c4c3)ccc2c1]

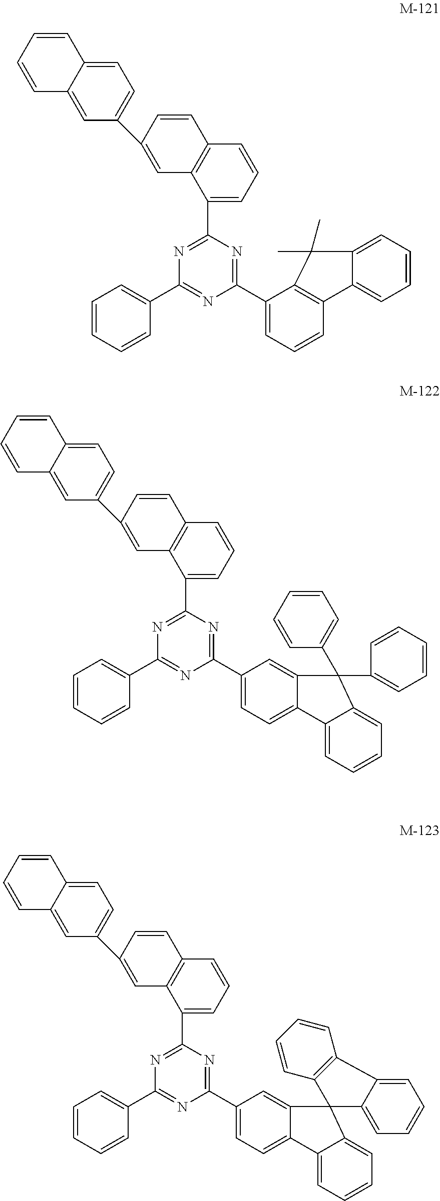 [SMILES: CC1(C)c2ccccc2-c2cccc(-c3nc(-c4ccccc4)nc(-c4cccc5ccc(-c6ccc7ccccc7c6)cc45)n3)c21 ; c1ccc(-c2nc(-c3ccc4c(c3)C(c3ccccc3)(c3ccccc3)c3ccccc3-4)nc(-c3cccc4ccc(-c5ccc6ccccc6c5)cc34)n2)cc1 ; c1ccc(-c2nc(-c3ccc4c(c3)C3(c5ccccc5-c5ccccc53)c3ccccc3-4)nc(-c3cccc4ccc(-c5ccc6ccccc6c5)cc34)n2)cc1]

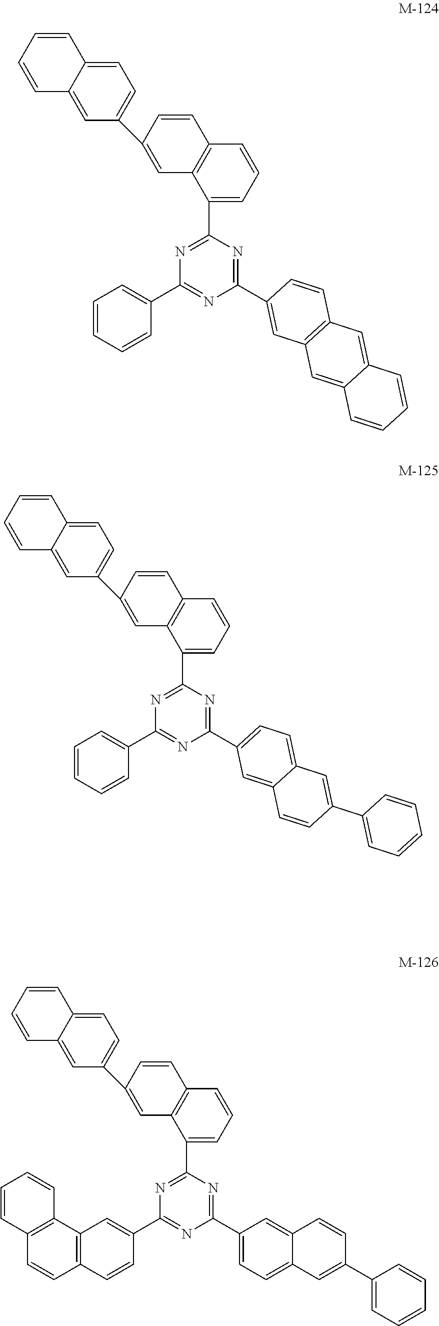 [SMILES: c1ccc(-c2nc(-c3ccc4cc5ccccc5cc4c3)nc(-c3cccc4ccc(-c5ccc6ccccc6c5)cc34)n2)cc1 ; c1ccc(-c2ccc3cc(-c4nc(-c5ccccc5)nc(-c5cccc6ccc(-c7ccc8ccccc8c7)cc56)n4)ccc3c2)cc1 ; c1ccc(-c2ccc3cc(-c4nc(-c5ccc6ccc7ccccc7c6c5)nc(-c5cccc6ccc(-c7ccc8ccccc8c7)cc56)n4)ccc3c2)cc1]

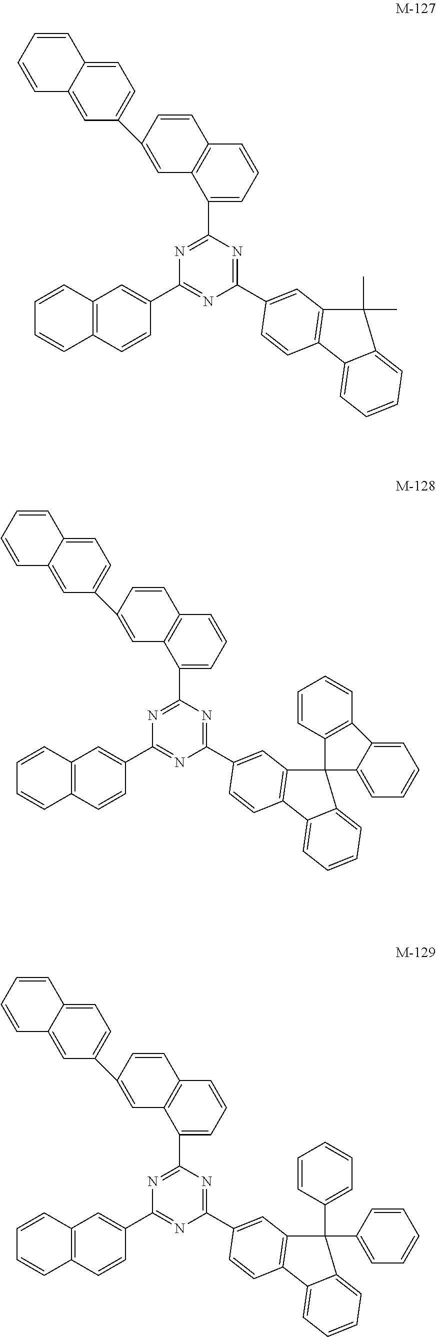 [SMILES: CC1(C)c2ccccc2-c2ccc(-c3nc(-c4ccc5ccccc5c4)nc(-c4cccc5ccc(-c6ccc7ccccc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7ccccc7c6)cc45)n3)cc21 ; c1ccc(C2(c3ccccc3)c3ccccc3-c3ccc(-c4nc(-c5ccc6ccccc6c5)nc(-c5cccc6ccc(-c7ccc8ccccc8c7)cc56)n4)cc32)cc1]

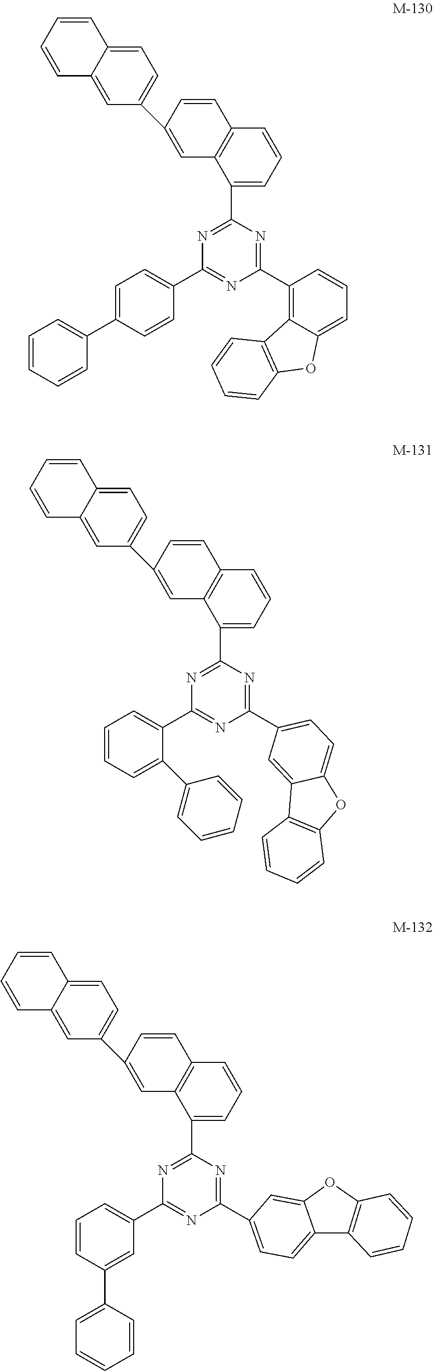 [SMILES: c1ccc(-c2ccc(-c3nc(-c4cccc5ccc(-c6ccc7ccccc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6ccccc6c5)cc34)n2)cc1 ; c1ccc(-c2cccc(-c3nc(-c4ccc5c(c4)oc4ccccc45)nc(-c4cccc5ccc(-c6ccc7ccccc7c6)cc45)n3)c2)cc1]

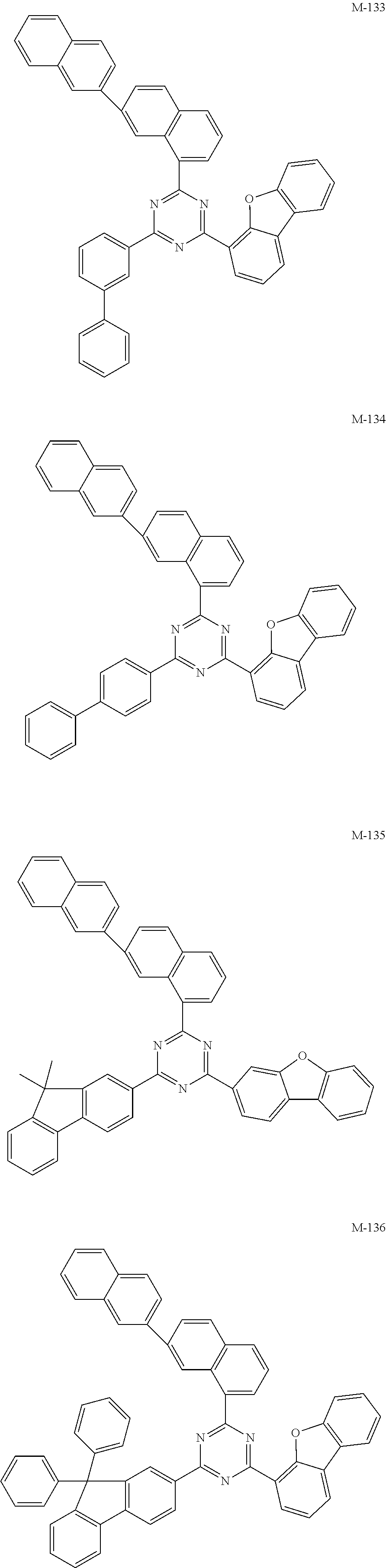 [SMILES: c1ccc(-c2cccc(-c3nc(-c4cccc5ccc(-c6ccc7ccccc7c6)cc45)nc(-c4cccc5c4oc4ccccc45)n3)c2)cc1 ; c1ccc(-c2ccc(-c3nc(-c4cccc5ccc(-c6ccc7ccccc7c6)cc45)nc(-c4cccc5c4oc4ccccc45)n3)cc2)cc1 ; CC1(C)c2ccccc2-c2ccc(-c3nc(-c4ccc5c(c4)oc4ccccc45)nc(-c4cccc5ccc(-c6ccc7ccccc7c6)cc45)n3)cc21 ; c1ccc(C2(c3ccccc3)c3ccccc3-c3ccc(-c4nc(-c5cccc6ccc(-c7ccc8ccccc8c7)cc56)nc(-c5cccc6c5oc5ccccc56)n4)cc32)cc1]

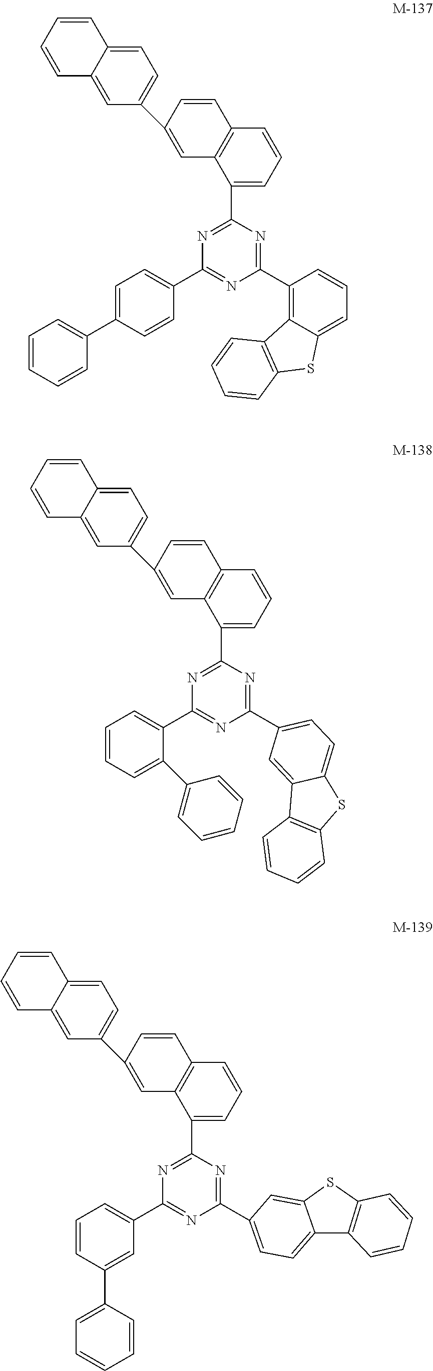 [SMILES: c1ccc(-c2ccc(-c3nc(-c4cccc5ccc(-c6ccc7ccccc7c6)cc45)nc(-c4cccc5sc6ccccc6c45)n3)cc2)cc1 ; c1ccc(-c2ccccc2-c2nc(-c3ccc4sc5ccccc5c4c3)nc(-c3cccc4ccc(-c5ccc6ccccc6c5)cc34)n2)cc1 ; c1ccc(-c2cccc(-c3nc(-c4ccc5c(c4)sc4ccccc45)nc(-c4cccc5ccc(-c6ccc7ccccc7c6)cc45)n3)c2)cc1]

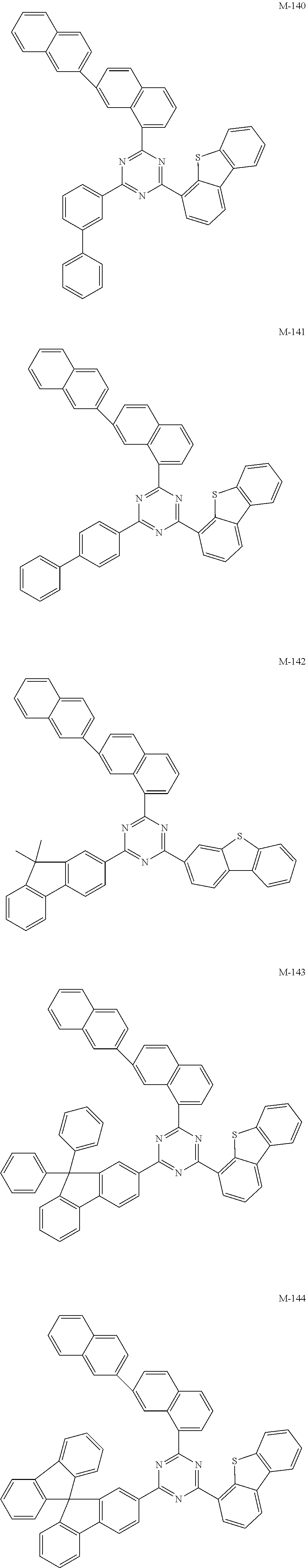 [SMILES: c1ccc(-c2cccc(-c3nc(-c4cccc5ccc(-c6ccc7ccccc7c6)cc45)nc(-c4cccc5c4sc4ccccc45)n3)c2)cc1 ; c1ccc(-c2ccc(-c3nc(-c4cccc5ccc(-c6ccc7ccccc7c6)cc45)nc(-c4cccc5c4sc4ccccc45)n3)cc2)cc1 ; CC1(C)c2ccccc2-c2ccc(-c3nc(-c4ccc5c(c4)sc4ccccc45)nc(-c4cccc5ccc(-c6ccc7ccccc7c6)cc45)n3)cc21 ; c1ccc(C2(c3ccccc3)c3ccccc3-c3ccc(-c4nc(-c5cccc6ccc(-c7ccc8ccccc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7ccccc7c6)cc45)nc(-c4cccc5c4sc4ccccc45)n3)cc21]

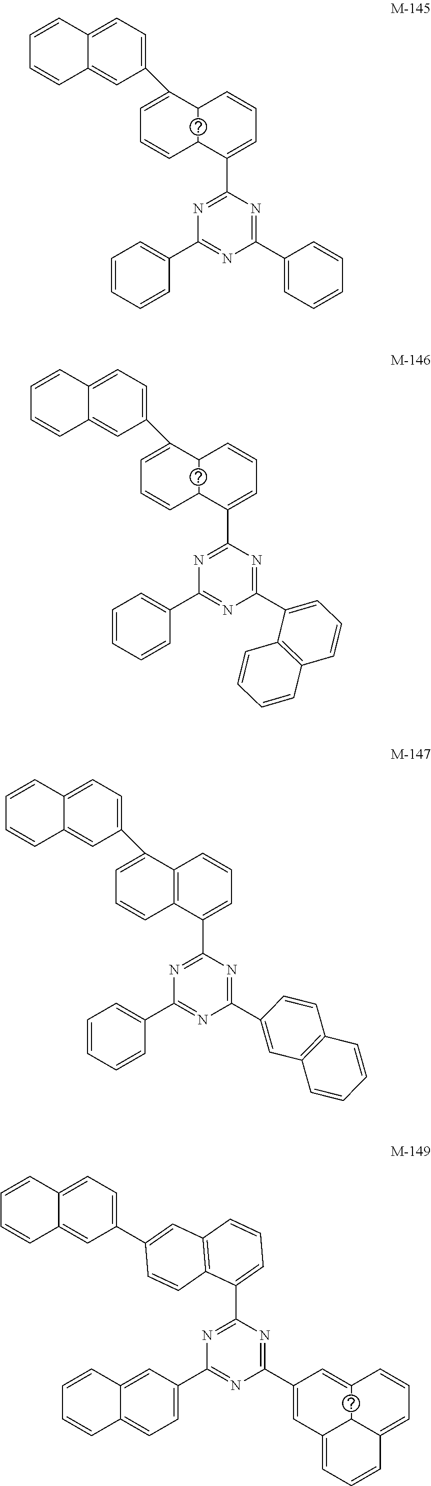 [SMILES: C1=CC2C(c3ccc4ccccc4c3)=CC=CC2C(c2nc(-c3ccccc3)nc(-c3ccccc3)n2)=C1 ; C1=CC2C(c3ccc4ccccc4c3)=CC=CC2C(c2nc(-c3ccccc3)nc(-c3cccc4ccccc34)n2)=C1 ; c1ccc(-c2nc(-c3ccc4ccccc4c3)nc(-c3cccc4c(-c5ccc6ccccc6c5)cccc34)n2)cc1 ; C1=CC2=CC=CC3=CC(c4nc(-c5ccc6ccccc6c5)nc(-c5cccc6cc(-c7ccc8ccccc8c7)ccc56)n4)=CC(=C1)C23]

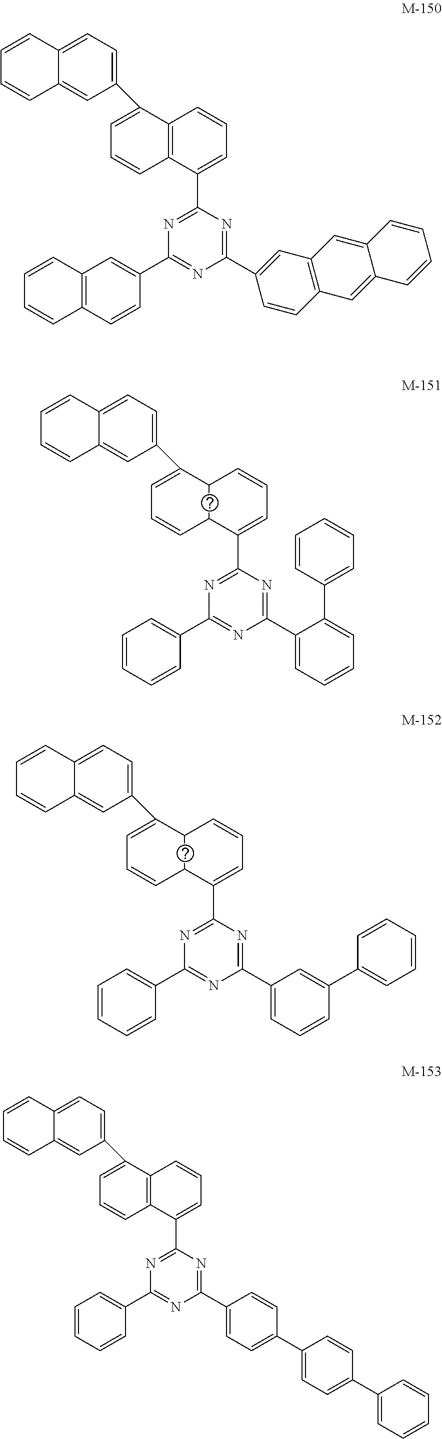 [SMILES: c1ccc2cc(-c3nc(-c4ccc5cc6ccccc6cc5c4)nc(-c4cccc5c(-c6ccc7ccccc7c6)cccc45)n3)ccc2c1 ; C1=CC2C(c3ccc4ccccc4c3)=CC=CC2C(c2nc(-c3ccccc3)nc(-c3ccccc3-c3ccccc3)n2)=C1 ; C1=CC2C(c3ccc4ccccc4c3)=CC=CC2C(c2nc(-c3ccccc3)nc(-c3cccc(-c4ccccc4)c3)n2)=C1 ; c1ccc(-c2ccc(-c3ccc(-c4nc(-c5ccccc5)nc(-c5cccc6c(-c7ccc8ccccc8c7)cccc56)n4)cc3)cc2)cc1]

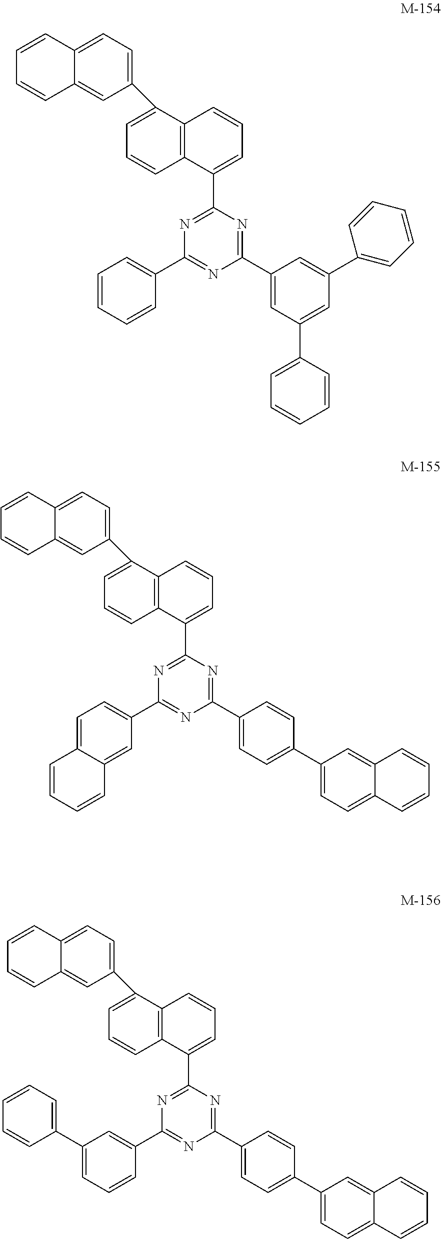 [SMILES: c1ccc(-c2cc(-c3ccccc3)cc(-c3nc(-c4ccccc4)nc(-c4cccc5c(-c6ccc7ccccc7c6)cccc45)n3)c2)cc1 ; c1ccc2cc(-c3ccc(-c4nc(-c5ccc6ccccc6c5)nc(-c5cccc6c(-c7ccc8ccccc8c7)cccc56)n4)cc3)ccc2c1 ; c1ccc(-c2cccc(-c3nc(-c4ccc(-c5ccc6ccccc6c5)cc4)nc(-c4cccc5c(-c6ccc7ccccc7c6)cccc45)n3)c2)cc1]

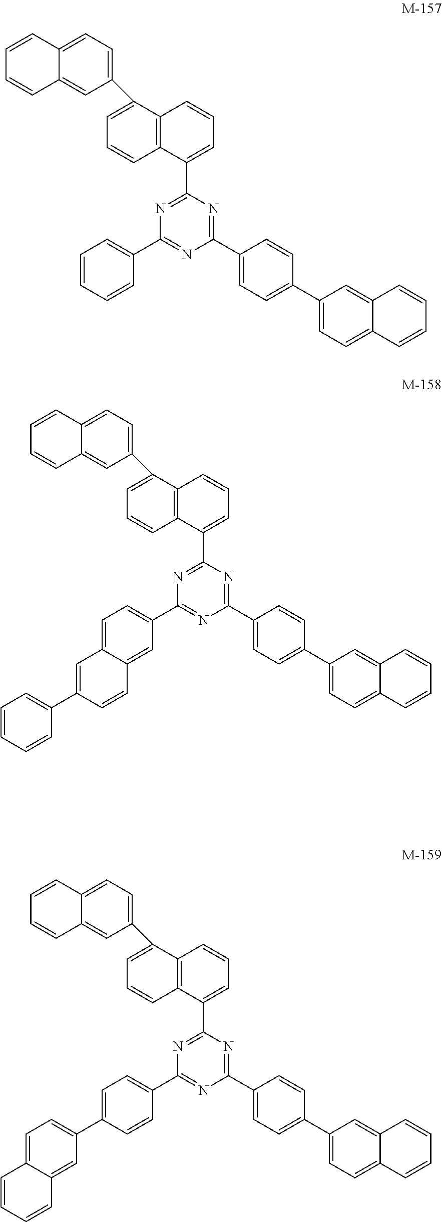 [SMILES: c1ccc(-c2nc(-c3ccc(-c4ccc5ccccc5c4)cc3)nc(-c3cccc4c(-c5ccc6ccccc6c5)cccc34)n2)cc1 ; c1ccc(-c2ccc3cc(-c4nc(-c5ccc(-c6ccc7ccccc7c6)cc5)nc(-c5cccc6c(-c7ccc8ccccc8c7)cccc56)n4)ccc3c2)cc1 ; c1ccc2cc(-c3ccc(-c4nc(-c5ccc(-c6ccc7ccccc7c6)cc5)nc(-c5cccc6c(-c7ccc8ccccc8c7)cccc56)n4)cc3)ccc2c1]

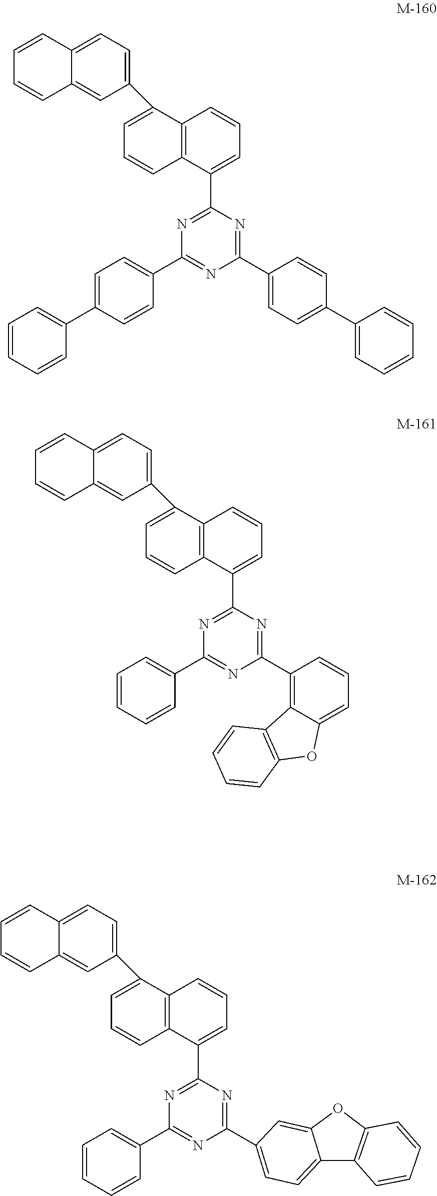 [SMILES: c1ccc(-c2ccc(-c3nc(-c4ccc(-c5ccccc5)cc4)nc(-c4cccc5c(-c6ccc7ccccc7c6)cccc45)n3)cc2)cc1 ; c1ccc(-c2nc(-c3cccc4c(-c5ccc6ccccc6c5)cccc34)nc(-c3cccc4oc5ccccc5c34)n2)cc1 ; c1ccc(-c2nc(-c3ccc4c(c3)oc3ccccc34)nc(-c3cccc4c(-c5ccc6ccccc6c5)cccc34)n2)cc1]

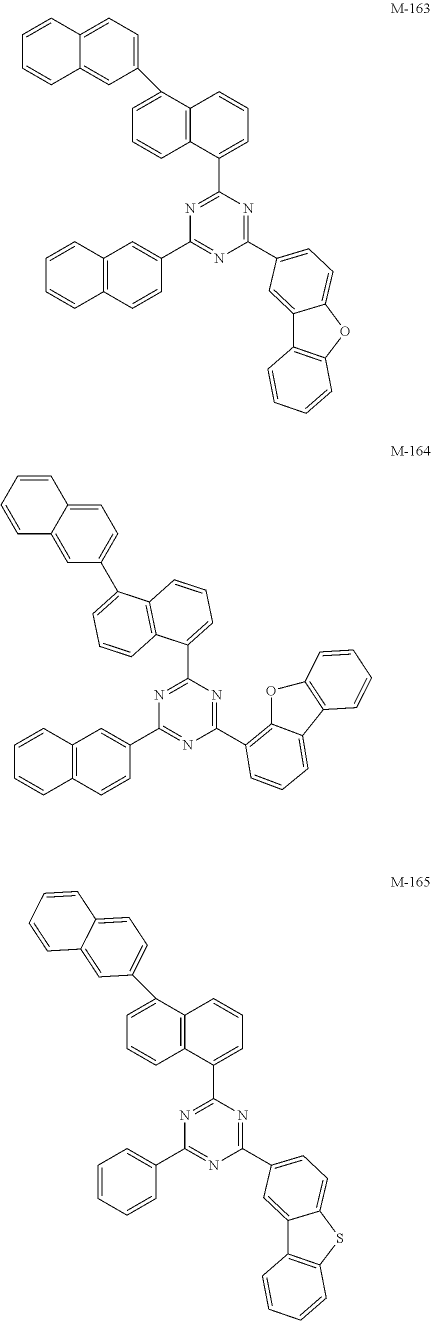 [SMILES: c1ccc2cc(-c3nc(-c4ccc5oc6ccccc6c5c4)nc(-c4cccc5c(-c6ccc7ccccc7c6)cccc45)n3)ccc2c1 ; c1ccc2cc(-c3nc(-c4cccc5c(-c6ccc7ccccc7c6)cccc45)nc(-c4cccc5c4oc4ccccc45)n3)ccc2c1 ; c1ccc(-c2nc(-c3ccc4sc5ccccc5c4c3)nc(-c3cccc4c(-c5ccc6ccccc6c5)cccc34)n2)cc1]

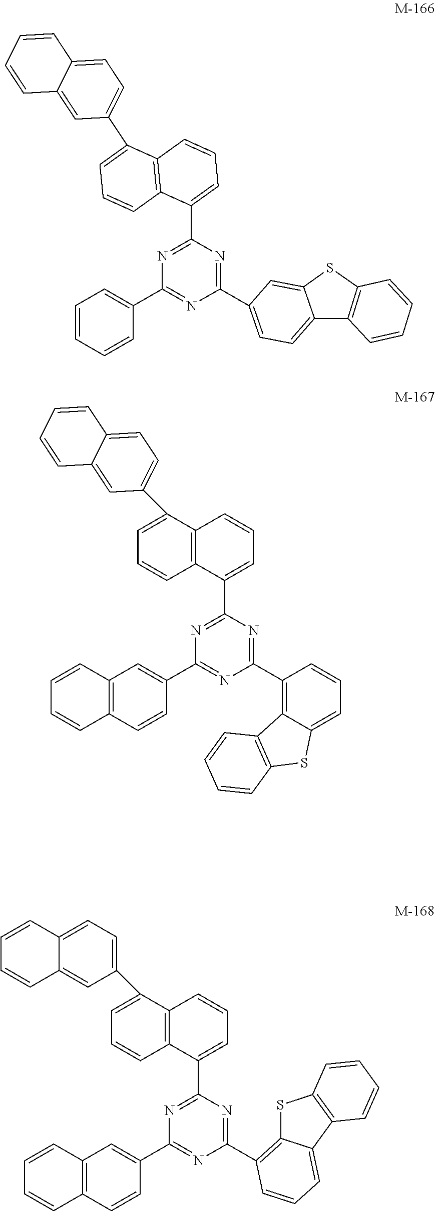 [SMILES: c1ccc(-c2nc(-c3ccc4c(c3)sc3ccccc34)nc(-c3cccc4c(-c5ccc6ccccc6c5)cccc34)n2)cc1 ; c1ccc2cc(-c3nc(-c4cccc5c(-c6ccc7ccccc7c6)cccc45)nc(-c4cccc5sc6ccccc6c45)n3)ccc2c1 ; c1ccc2cc(-c3nc(-c4cccc5c(-c6ccc7ccccc7c6)cccc45)nc(-c4cccc5c4sc4ccccc45)n3)ccc2c1]

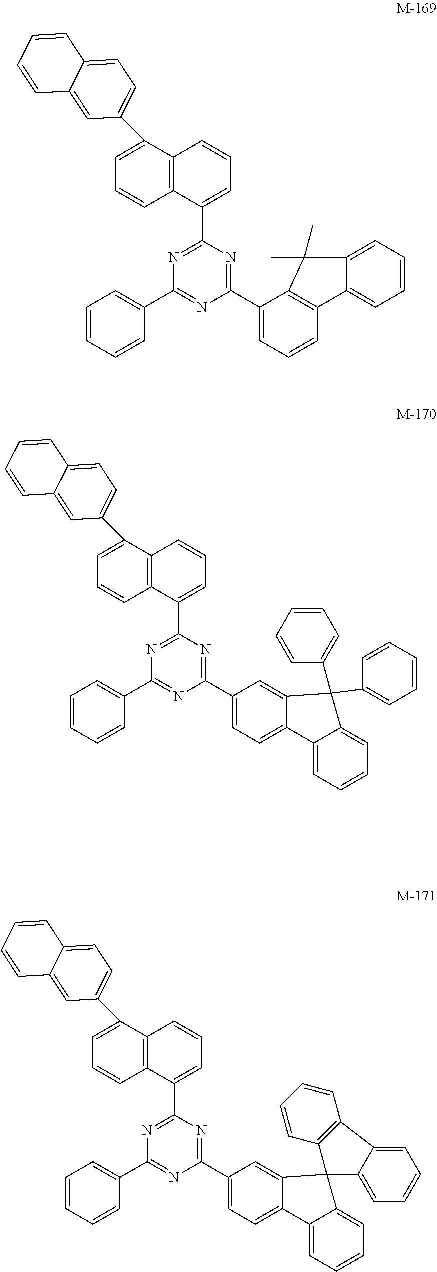 [SMILES: CC1(C)c2ccccc2-c2cccc(-c3nc(-c4ccccc4)nc(-c4cccc5c(-c6ccc7ccccc7c6)cccc45)n3)c21 ; c1ccc(-c2nc(-c3ccc4c(c3)C(c3ccccc3)(c3ccccc3)c3ccccc3-4)nc(-c3cccc4c(-c5ccc6ccccc6c5)cccc34)n2)cc1 ; c1ccc(-c2nc(-c3ccc4c(c3)C3(c5ccccc5-c5ccccc53)c3ccccc3-4)nc(-c3cccc4c(-c5ccc6ccccc6c5)cccc34)n2)cc1]

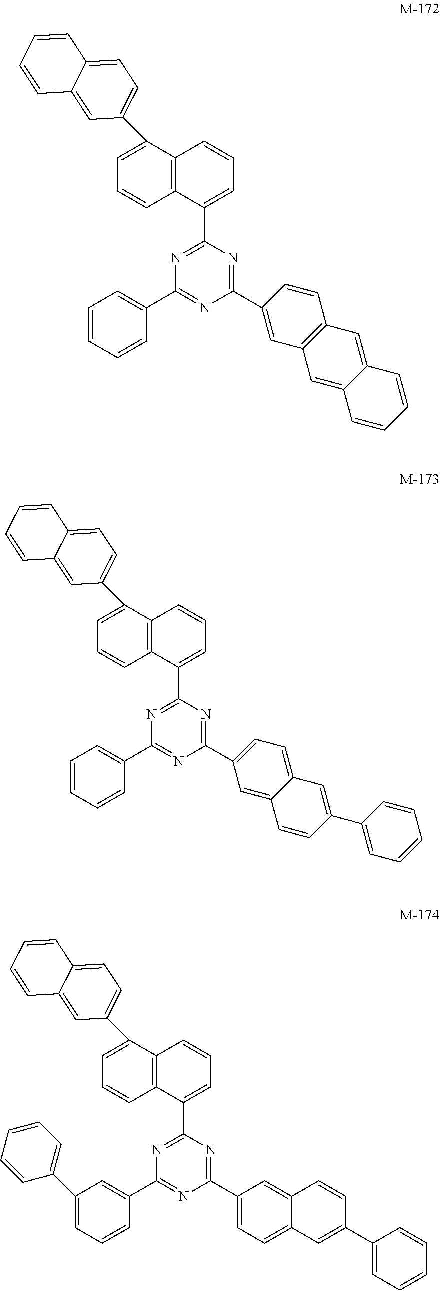 [SMILES: c1ccc(-c2nc(-c3ccc4cc5ccccc5cc4c3)nc(-c3cccc4c(-c5ccc6ccccc6c5)cccc34)n2)cc1 ; c1ccc(-c2ccc3cc(-c4nc(-c5ccccc5)nc(-c5cccc6c(-c7ccc8ccccc8c7)cccc56)n4)ccc3c2)cc1 ; c1ccc(-c2cccc(-c3nc(-c4ccc5cc(-c6ccccc6)ccc5c4)nc(-c4cccc5c(-c6ccc7ccccc7c6)cccc45)n3)c2)cc1]

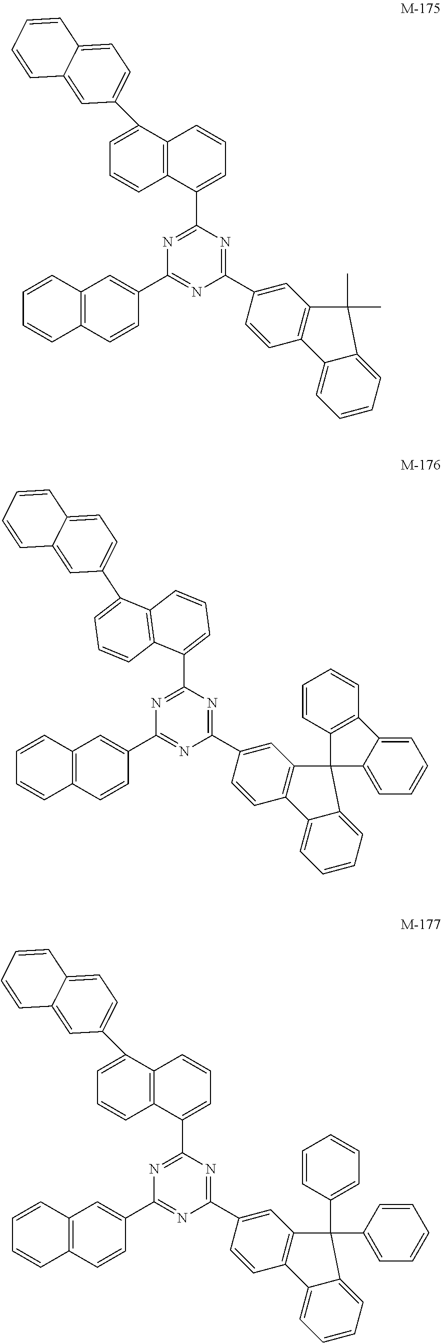 [SMILES: CC1(C)c2ccccc2-c2ccc(-c3nc(-c4ccc5ccccc5c4)nc(-c4cccc5c(-c6ccc7ccccc7c6)cccc45)n3)cc21 ; c1ccc2c(c1)-c1ccccc1C21c2ccccc2-c2ccc(-c3nc(-c4ccc5ccccc5c4)nc(-c4cccc5c(-c6ccc7ccccc7c6)cccc45)n3)cc21 ; c1ccc(C2(c3ccccc3)c3ccccc3-c3ccc(-c4nc(-c5ccc6ccccc6c5)nc(-c5cccc6c(-c7ccc8ccccc8c7)cccc56)n4)cc32)cc1]

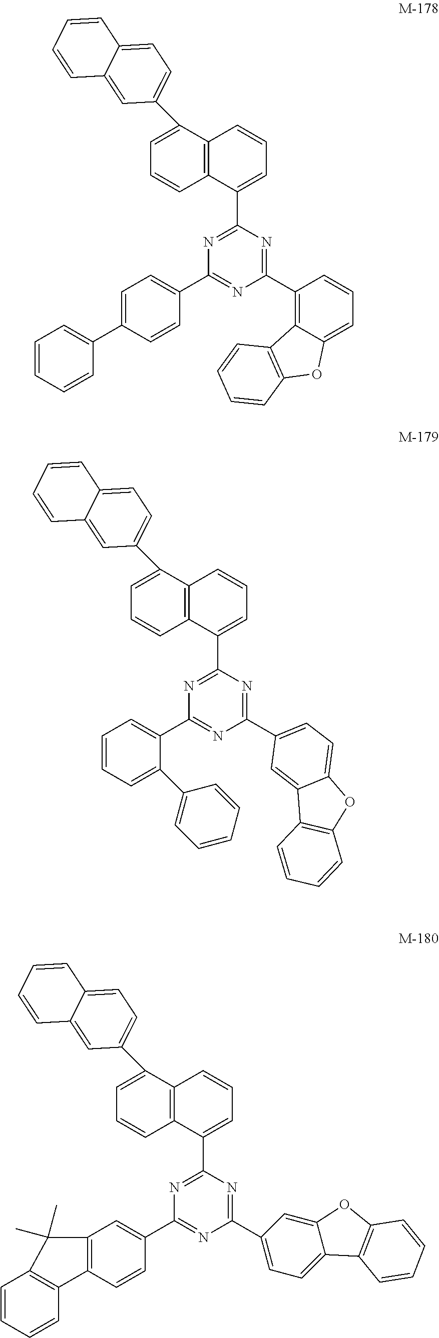 [SMILES: c1ccc(-c2ccc(-c3nc(-c4cccc5c(-c6ccc7ccccc7c6)cccc45)nc(-c4cccc5oc6ccccc6c45)n3)cc2)cc1 ; c1ccc(-c2ccccc2-c2nc(-c3ccc4oc5ccccc5c4c3)nc(-c3cccc4c(-c5ccc6ccccc6c5)cccc34)n2)cc1 ; CC1(C)c2ccccc2-c2ccc(-c3nc(-c4ccc5c(c4)oc4ccccc45)nc(-c4cccc5c(-c6ccc7ccccc7c6)cccc45)n3)cc21]

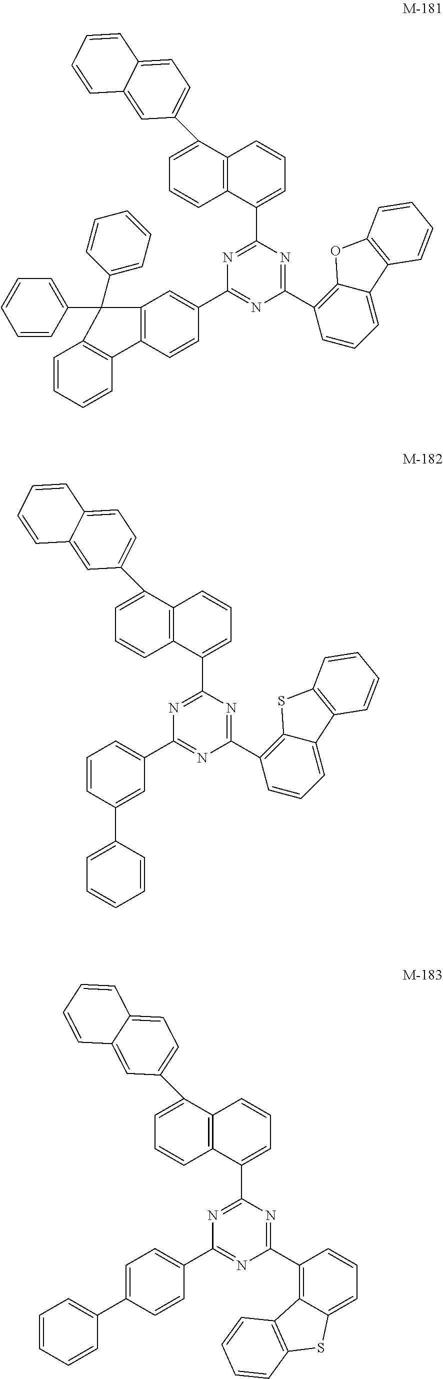 [SMILES: c1ccc(C2(c3ccccc3)c3ccccc3-c3ccc(-c4nc(-c5cccc6c(-c7ccc8ccccc8c7)cccc56)nc(-c5cccc6c5oc5ccccc56)n4)cc32)cc1 ; c1ccc(-c2cccc(-c3nc(-c4cccc5c(-c6ccc7ccccc7c6)cccc45)nc(-c4cccc5c4sc4ccccc45)n3)c2)cc1 ; c1ccc(-c2ccc(-c3nc(-c4cccc5c(-c6ccc7ccccc7c6)cccc45)nc(-c4cccc5sc6ccccc6c45)n3)cc2)cc1]

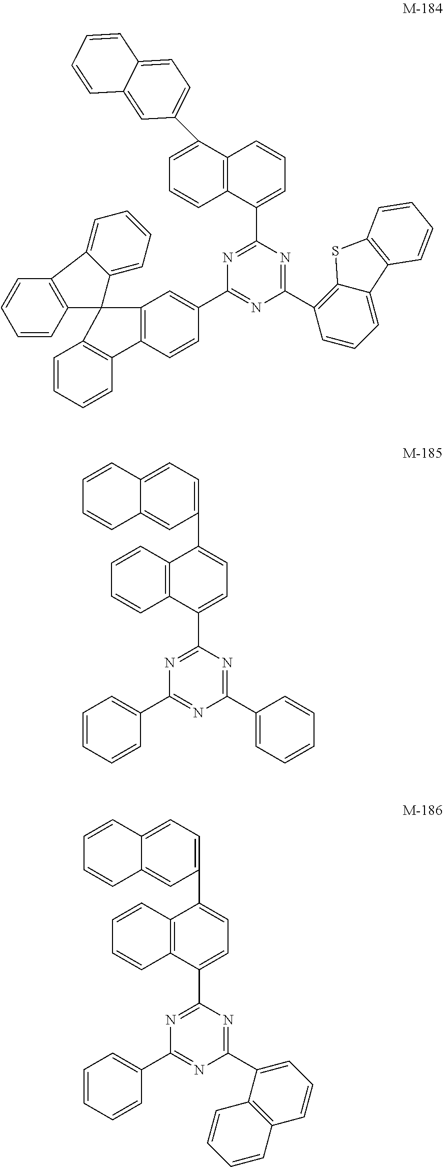 [SMILES: c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21 ; c1ccc(-c2nc(-c3ccccc3)nc(-c3ccc(-c4ccc5ccccc5c4)c4ccccc34)n2)cc1 ; c1ccc(-c2nc(-c3cccc4ccccc34)nc(-c3ccc(-c4ccc5ccccc5c4)c4ccccc34)n2)cc1]

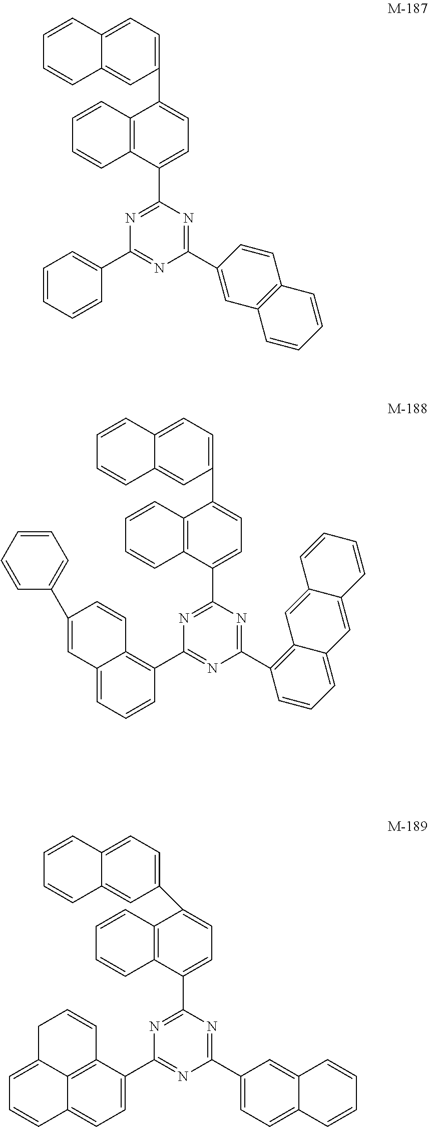 [SMILES: c1ccc(-c2nc(-c3ccc4ccccc4c3)nc(-c3ccc(-c4ccc5ccccc5c4)c4ccccc34)n2)cc1 ; c1ccc(-c2ccc3c(-c4nc(-c5cccc6cc7ccccc7cc56)nc(-c5ccc(-c6ccc7ccccc7c6)c6ccccc56)n4)cccc3c2)cc1 ; C1=Cc2c(-c3nc(-c4ccc5ccccc5c4)nc(-c4ccc(-c5ccc6ccccc6c5)c5ccccc45)n3)ccc3cccc(c23)C1]

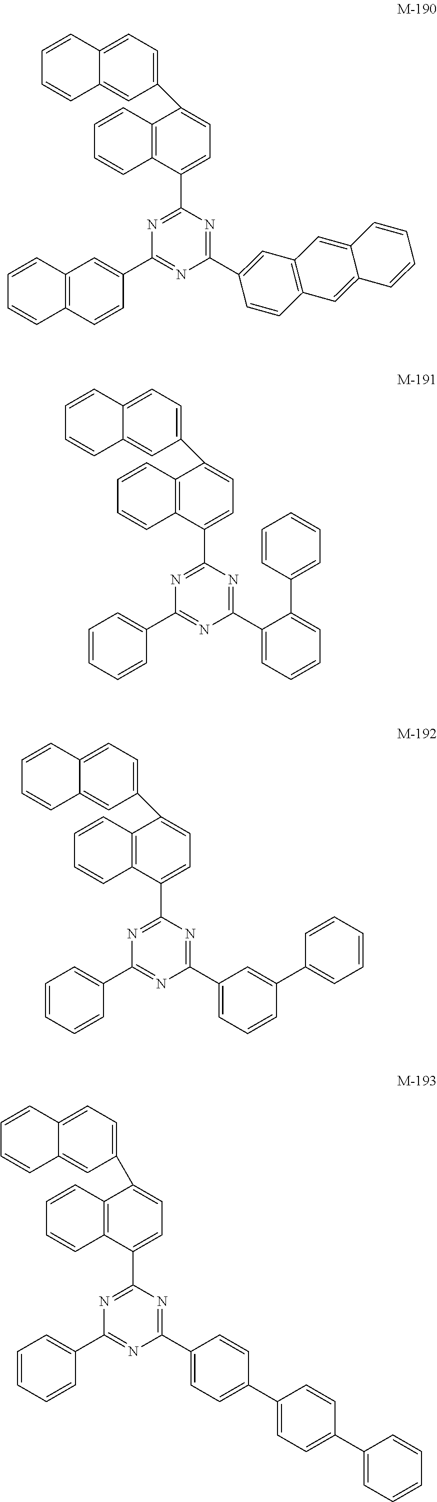 [SMILES: c1ccc2cc(-c3nc(-c4ccc5cc6ccccc6cc5c4)nc(-c4ccc(-c5ccc6ccccc6c5)c5ccccc45)n3)ccc2c1 ; c1ccc(-c2nc(-c3ccccc3-c3ccccc3)nc(-c3ccc(-c4ccc5ccccc5c4)c4ccccc34)n2)cc1 ; c1ccc(-c2cccc(-c3nc(-c4ccccc4)nc(-c4ccc(-c5ccc6ccccc6c5)c5ccccc45)n3)c2)cc1 ; c1ccc(-c2ccc(-c3ccc(-c4nc(-c5ccccc5)nc(-c5ccc(-c6ccc7ccccc7c6)c6ccccc56)n4)cc3)cc2)cc1]

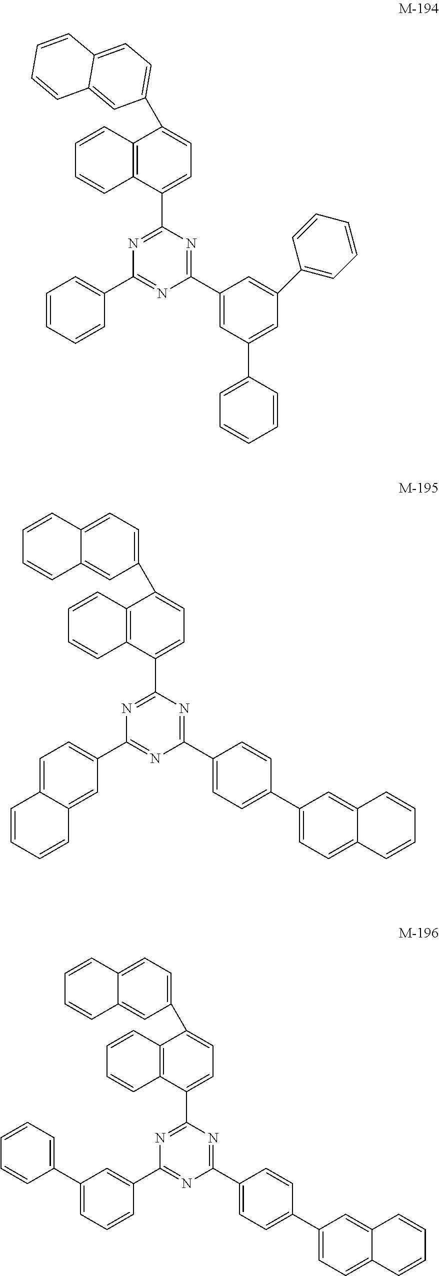 [SMILES: c1ccc(-c2cc(-c3ccccc3)cc(-c3nc(-c4ccccc4)nc(-c4ccc(-c5ccc6ccccc6c5)c5ccccc45)n3)c2)cc1 ; c1ccc2cc(-c3ccc(-c4nc(-c5ccc6ccccc6c5)nc(-c5ccc(-c6ccc7ccccc7c6)c6ccccc56)n4)cc3)ccc2c1 ; c1ccc(-c2cccc(-c3nc(-c4ccc(-c5ccc6ccccc6c5)cc4)nc(-c4ccc(-c5ccc6ccccc6c5)c5ccccc45)n3)c2)cc1]

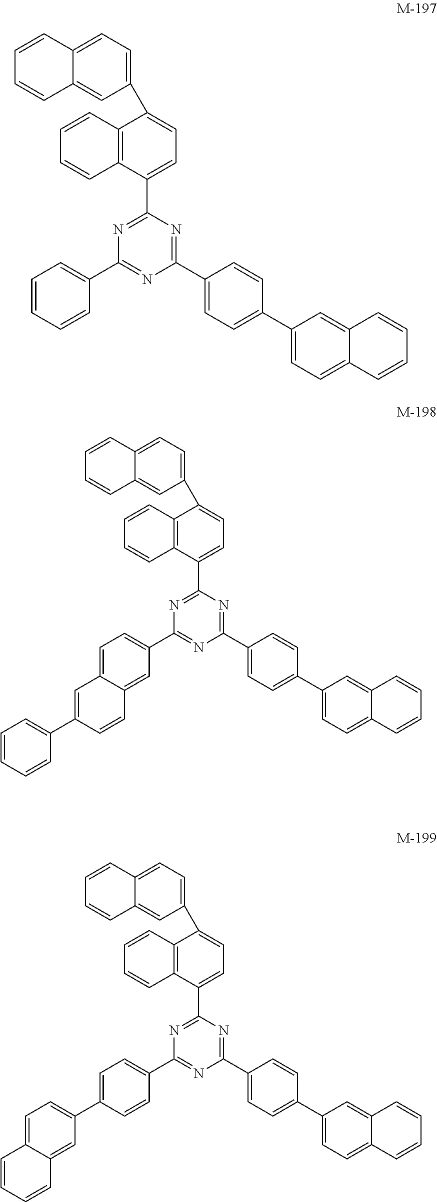 [SMILES: c1ccc(-c2nc(-c3ccc(-c4ccc5ccccc5c4)cc3)nc(-c3ccc(-c4ccc5ccccc5c4)c4ccccc34)n2)cc1 ; c1ccc(-c2ccc3cc(-c4nc(-c5ccc(-c6ccc7ccccc7c6)cc5)nc(-c5ccc(-c6ccc7ccccc7c6)c6ccccc56)n4)ccc3c2)cc1 ; c1ccc2cc(-c3ccc(-c4nc(-c5ccc(-c6ccc7ccccc7c6)cc5)nc(-c5ccc(-c6ccc7ccccc7c6)c6ccccc56)n4)cc3)ccc2c1]

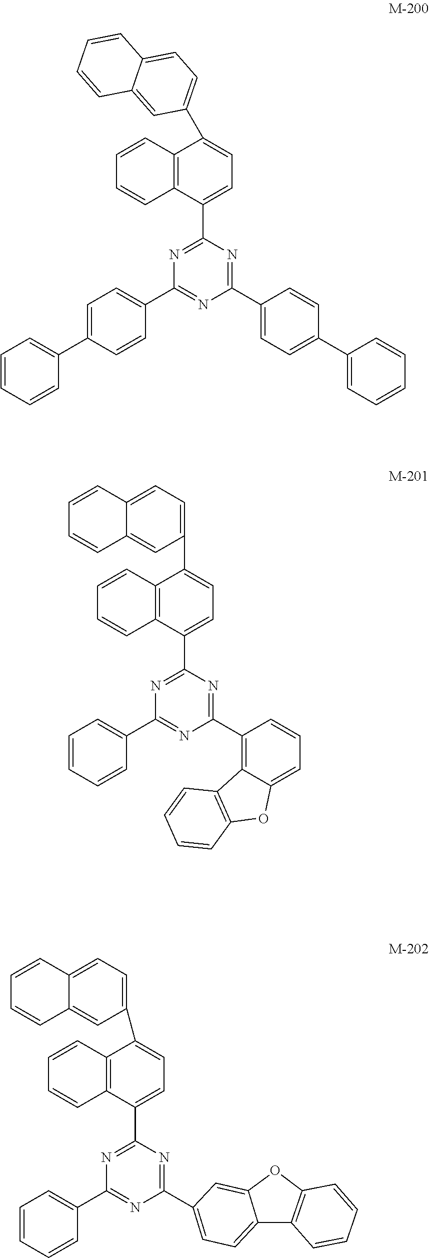 [SMILES: c1ccc(-c2ccc(-c3nc(-c4ccc(-c5ccccc5)cc4)nc(-c4ccc(-c5ccc6ccccc6c5)c5ccccc45)n3)cc2)cc1 ; c1ccc(-c2nc(-c3ccc(-c4ccc5ccccc5c4)c4ccccc34)nc(-c3cccc4oc5ccccc5c34)n2)cc1 ; c1ccc(-c2nc(-c3ccc4c(c3)oc3ccccc34)nc(-c3ccc(-c4ccc5ccccc5c4)c4ccccc34)n2)cc1]

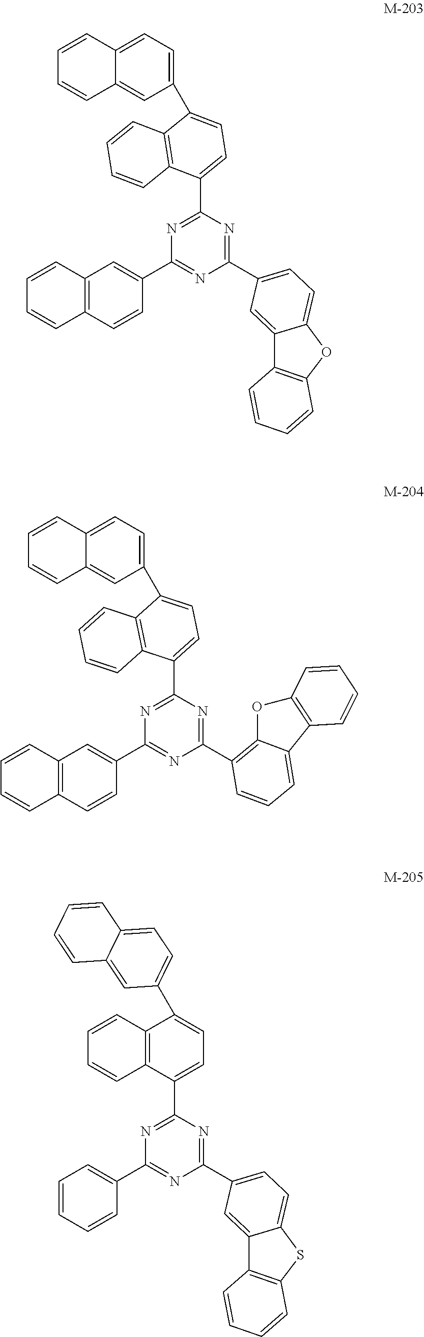 [SMILES: c1ccc2cc(-c3nc(-c4ccc5oc6ccccc6c5c4)nc(-c4ccc(-c5ccc6ccccc6c5)c5ccccc45)n3)ccc2c1 ; c1ccc2cc(-c3nc(-c4ccc(-c5ccc6ccccc6c5)c5ccccc45)nc(-c4cccc5c4oc4ccccc45)n3)ccc2c1 ; c1ccc(-c2nc(-c3ccc4sc5ccccc5c4c3)nc(-c3ccc(-c4ccc5ccccc5c4)c4ccccc34)n2)cc1]

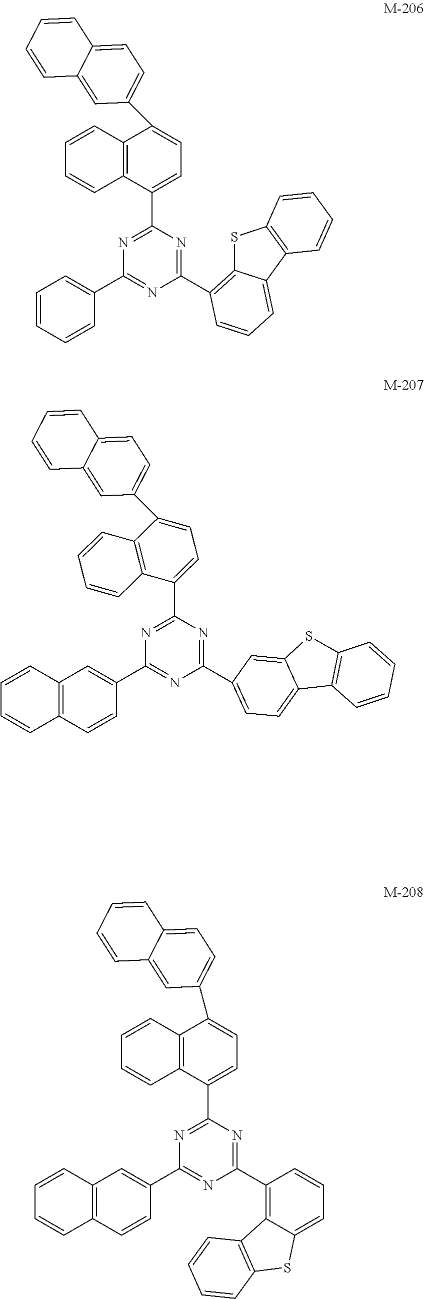 [SMILES: c1ccc(-c2nc(-c3ccc(-c4ccc5ccccc5c4)c4ccccc34)nc(-c3cccc4c3sc3ccccc34)n2)cc1 ; c1ccc2cc(-c3nc(-c4ccc5c(c4)sc4ccccc45)nc(-c4ccc(-c5ccc6ccccc6c5)c5ccccc45)n3)ccc2c1 ; c1ccc2cc(-c3nc(-c4ccc(-c5ccc6ccccc6c5)c5ccccc45)nc(-c4cccc5sc6ccccc6c45)n3)ccc2c1]

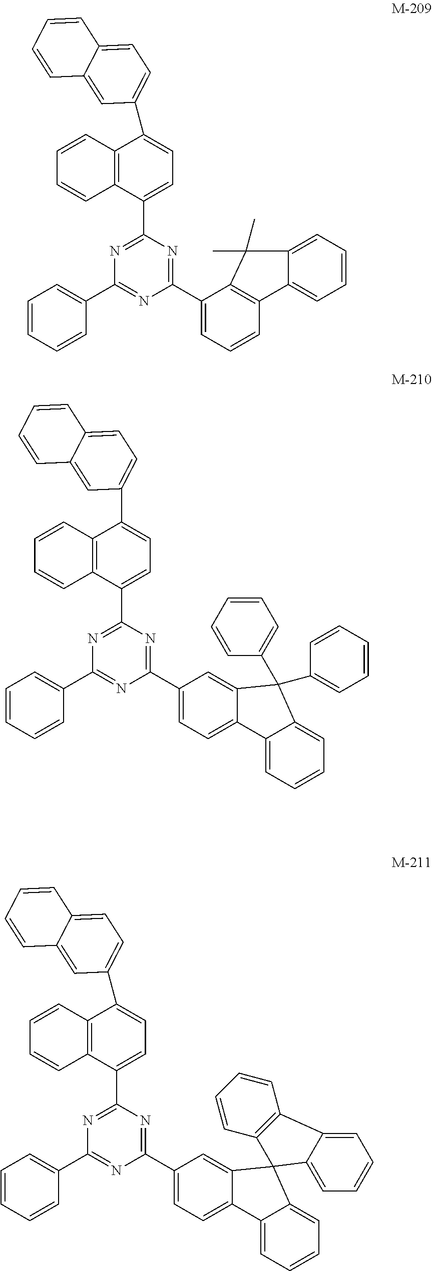 [SMILES: CC1(C)c2ccccc2-c2cccc(-c3nc(-c4ccccc4)nc(-c4ccc(-c5ccc6ccccc6c5)c5ccccc45)n3)c21 ; c1ccc(-c2nc(-c3ccc4c(c3)C(c3ccccc3)(c3ccccc3)c3ccccc3-4)nc(-c3ccc(-c4ccc5ccccc5c4)c4ccccc34)n2)cc1 ; c1ccc(-c2nc(-c3ccc4c(c3)C3(c5ccccc5-c5ccccc53)c3ccccc3-4)nc(-c3ccc(-c4ccc5ccccc5c4)c4ccccc34)n2)cc1]

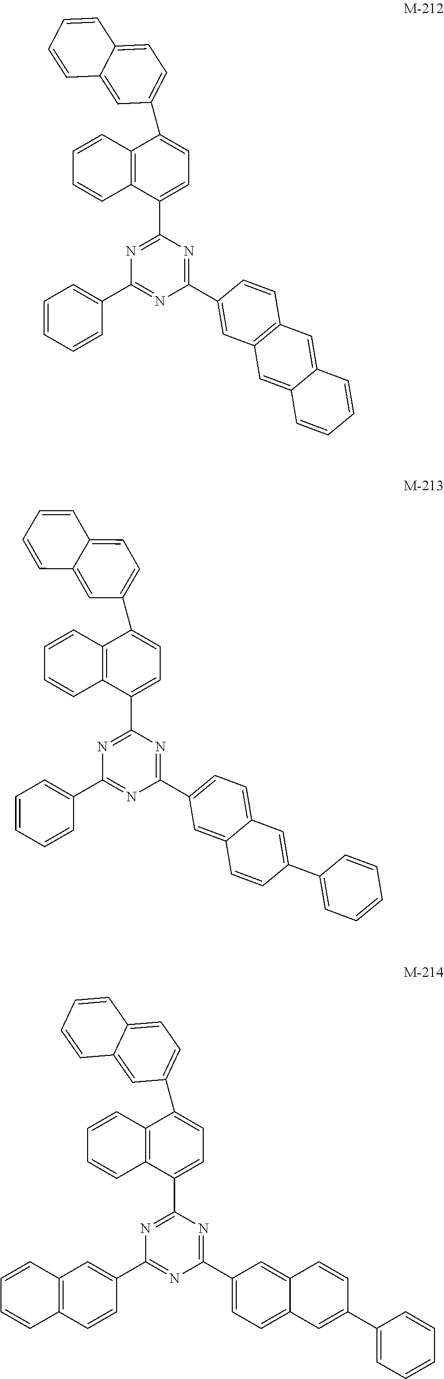 [SMILES: c1ccc(-c2nc(-c3ccc4cc5ccccc5cc4c3)nc(-c3ccc(-c4ccc5ccccc5c4)c4ccccc34)n2)cc1 ; c1ccc(-c2ccc3cc(-c4nc(-c5ccccc5)nc(-c5ccc(-c6ccc7ccccc7c6)c6ccccc56)n4)ccc3c2)cc1 ; c1ccc(-c2ccc3cc(-c4nc(-c5ccc6ccccc6c5)nc(-c5ccc(-c6ccc7ccccc7c6)c6ccccc56)n4)ccc3c2)cc1]

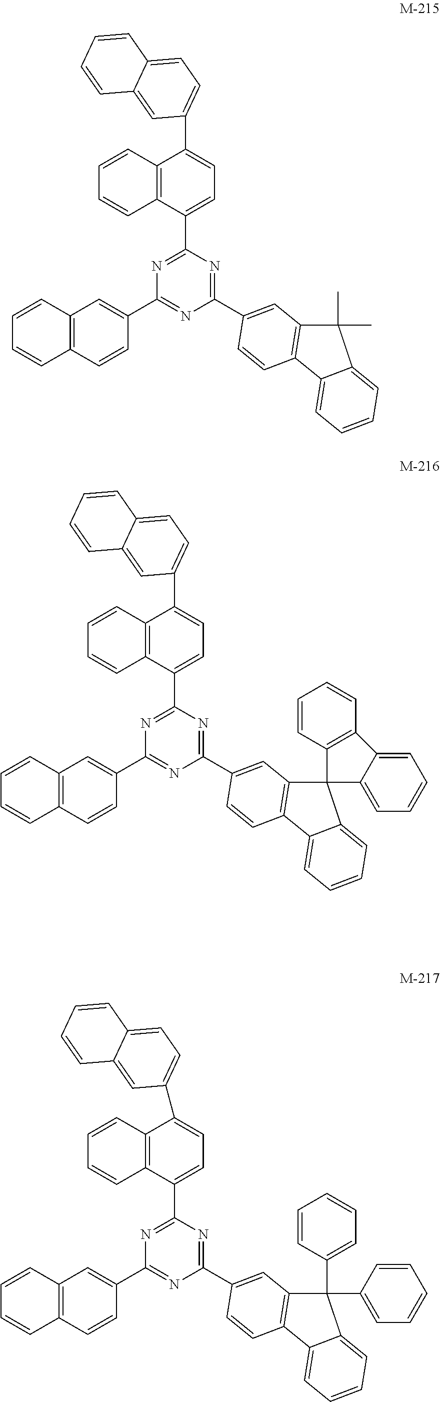 [SMILES: CC1(C)c2ccccc2-c2ccc(-c3nc(-c4ccc5ccccc5c4)nc(-c4ccc(-c5ccc6ccccc6c5)c5ccccc45)n3)cc21 ; c1ccc2c(c1)-c1ccccc1C21c2ccccc2-c2ccc(-c3nc(-c4ccc5ccccc5c4)nc(-c4ccc(-c5ccc6ccccc6c5)c5ccccc45)n3)cc21 ; c1ccc(C2(c3ccccc3)c3ccccc3-c3ccc(-c4nc(-c5ccc6ccccc6c5)nc(-c5ccc(-c6ccc7ccccc7c6)c6ccccc56)n4)cc32)cc1]

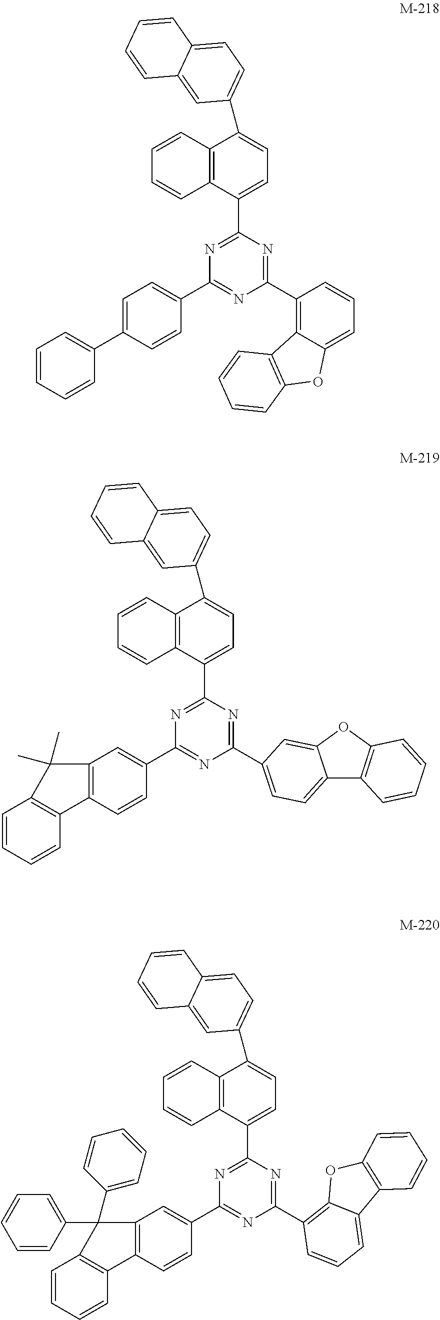 [SMILES: c1ccc(-c2ccc(-c3nc(-c4ccc(-c5ccc6ccccc6c5)c5ccccc45)nc(-c4cccc5oc6ccccc6c45)n3)cc2)cc1 ; CC1(C)c2ccccc2-c2ccc(-c3nc(-c4ccc5c(c4)oc4ccccc45)nc(-c4ccc(-c5ccc6ccccc6c5)c5ccccc45)n3)cc21 ; c1ccc(C2(c3ccccc3)c3ccccc3-c3ccc(-c4nc(-c5ccc(-c6ccc7ccccc7c6)c6ccccc56)nc(-c5cccc6c5oc5ccccc56)n4)cc32)cc1]

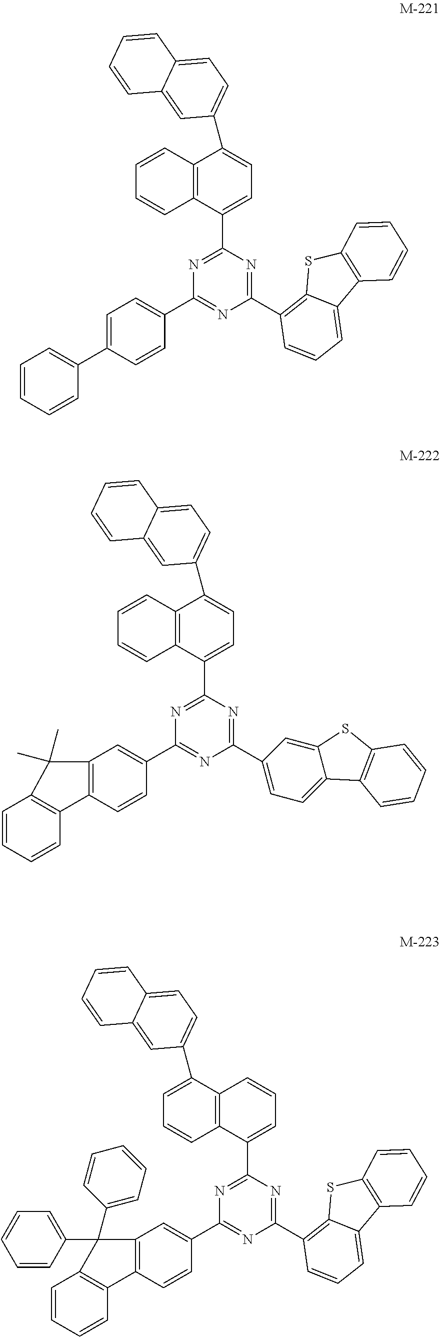 [SMILES: c1ccc(-c2ccc(-c3nc(-c4ccc(-c5ccc6ccccc6c5)c5ccccc45)nc(-c4cccc5c4sc4ccccc45)n3)cc2)cc1 ; CC1(C)c2ccccc2-c2ccc(-c3nc(-c4ccc5c(c4)sc4ccccc45)nc(-c4ccc(-c5ccc6ccccc6c5)c5ccccc45)n3)cc21 ; c1ccc(C2(c3ccccc3)c3ccccc3-c3ccc(-c4nc(-c5cccc6c(-c7ccc8ccccc8c7)cccc56)nc(-c5cccc6c5sc5ccccc56)n4)cc32)cc1]

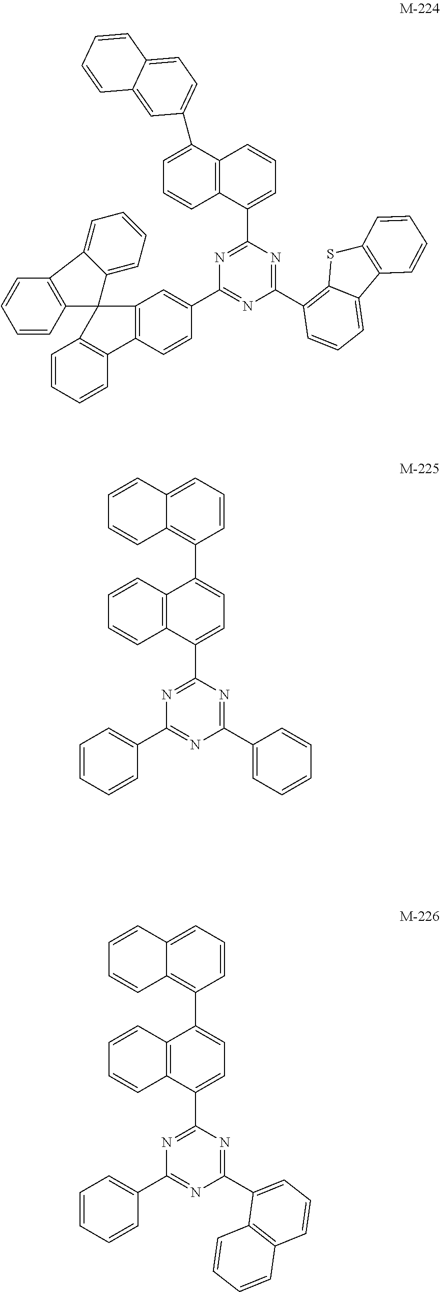 [SMILES: c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21 ; c1ccc(-c2nc(-c3ccccc3)nc(-c3ccc(-c4cccc5ccccc45)c4ccccc34)n2)cc1 ; c1ccc(-c2nc(-c3cccc4ccccc34)nc(-c3ccc(-c4cccc5ccccc45)c4ccccc34)n2)cc1]

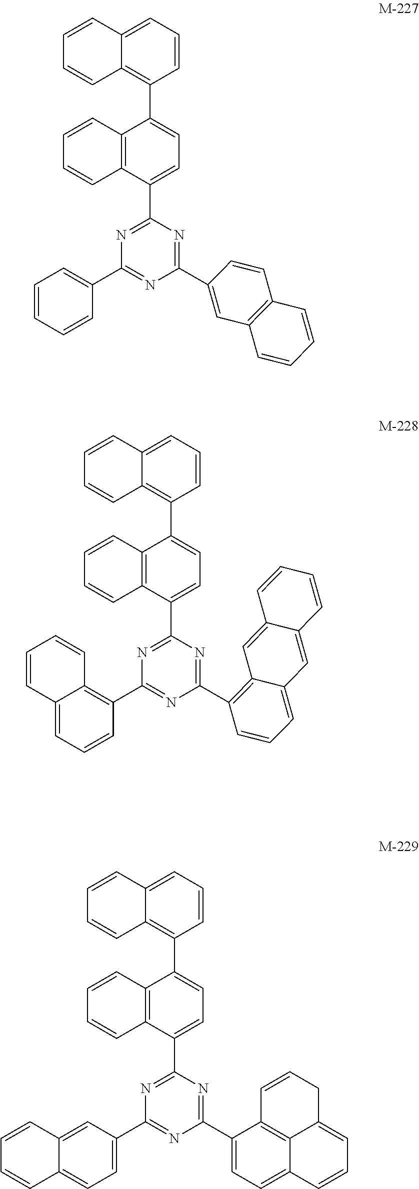 [SMILES: c1ccc(-c2nc(-c3ccc4ccccc4c3)nc(-c3ccc(-c4cccc5ccccc45)c4ccccc34)n2)cc1 ; c1ccc2cc3c(-c4nc(-c5cccc6ccccc56)nc(-c5ccc(-c6cccc7ccccc67)c6ccccc56)n4)cccc3cc2c1 ; C1=Cc2c(-c3nc(-c4ccc5ccccc5c4)nc(-c4ccc(-c5cccc6ccccc56)c5ccccc45)n3)ccc3cccc(c23)C1]

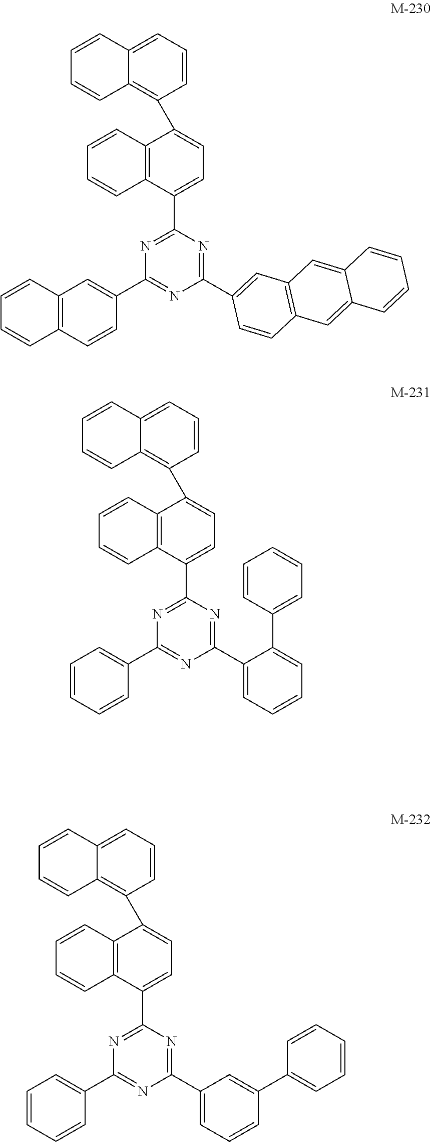 [SMILES: c1ccc2cc(-c3nc(-c4ccc5cc6ccccc6cc5c4)nc(-c4ccc(-c5cccc6ccccc56)c5ccccc45)n3)ccc2c1 ; c1ccc(-c2nc(-c3ccccc3-c3ccccc3)nc(-c3ccc(-c4cccc5ccccc45)c4ccccc34)n2)cc1 ; c1ccc(-c2cccc(-c3nc(-c4ccccc4)nc(-c4ccc(-c5cccc6ccccc56)c5ccccc45)n3)c2)cc1]

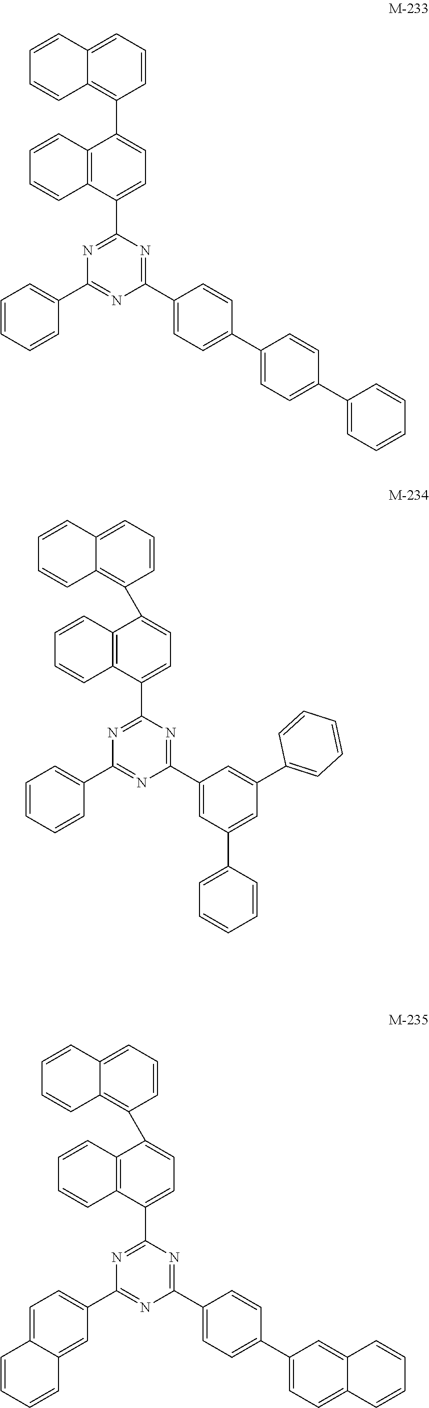 [SMILES: c1ccc(-c2ccc(-c3ccc(-c4nc(-c5ccccc5)nc(-c5ccc(-c6cccc7ccccc67)c6ccccc56)n4)cc3)cc2)cc1 ; c1ccc(-c2cc(-c3ccccc3)cc(-c3nc(-c4ccccc4)nc(-c4ccc(-c5cccc6ccccc56)c5ccccc45)n3)c2)cc1 ; c1ccc2cc(-c3ccc(-c4nc(-c5ccc6ccccc6c5)nc(-c5ccc(-c6cccc7ccccc67)c6ccccc56)n4)cc3)ccc2c1]

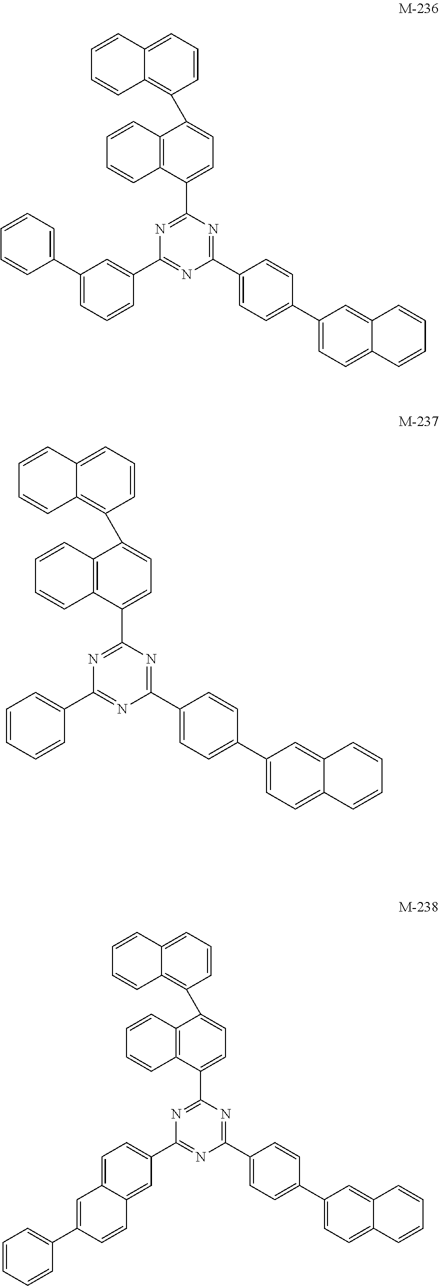 [SMILES: c1ccc(-c2cccc(-c3nc(-c4ccc(-c5ccc6ccccc6c5)cc4)nc(-c4ccc(-c5cccc6ccccc56)c5ccccc45)n3)c2)cc1 ; c1ccc(-c2nc(-c3ccc(-c4ccc5ccccc5c4)cc3)nc(-c3ccc(-c4cccc5ccccc45)c4ccccc34)n2)cc1 ; c1ccc(-c2ccc3cc(-c4nc(-c5ccc(-c6ccc7ccccc7c6)cc5)nc(-c5ccc(-c6cccc7ccccc67)c6ccccc56)n4)ccc3c2)cc1]

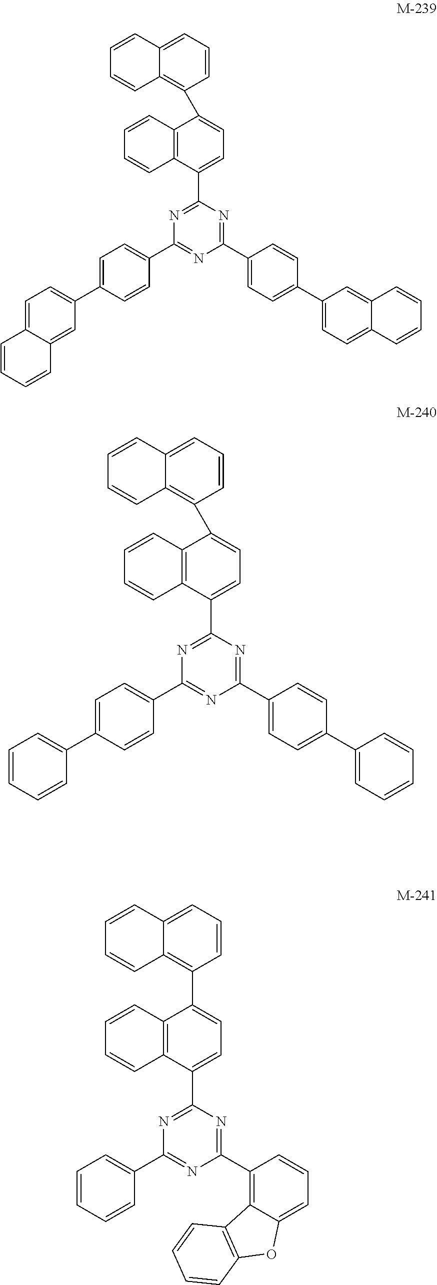 [SMILES: c1ccc2cc(-c3ccc(-c4nc(-c5ccc(-c6ccc7ccccc7c6)cc5)nc(-c5ccc(-c6cccc7ccccc67)c6ccccc56)n4)cc3)ccc2c1 ; c1ccc(-c2ccc(-c3nc(-c4ccc(-c5ccccc5)cc4)nc(-c4ccc(-c5cccc6ccccc56)c5ccccc45)n3)cc2)cc1 ; c1ccc(-c2nc(-c3ccc(-c4cccc5ccccc45)c4ccccc34)nc(-c3cccc4oc5ccccc5c34)n2)cc1]

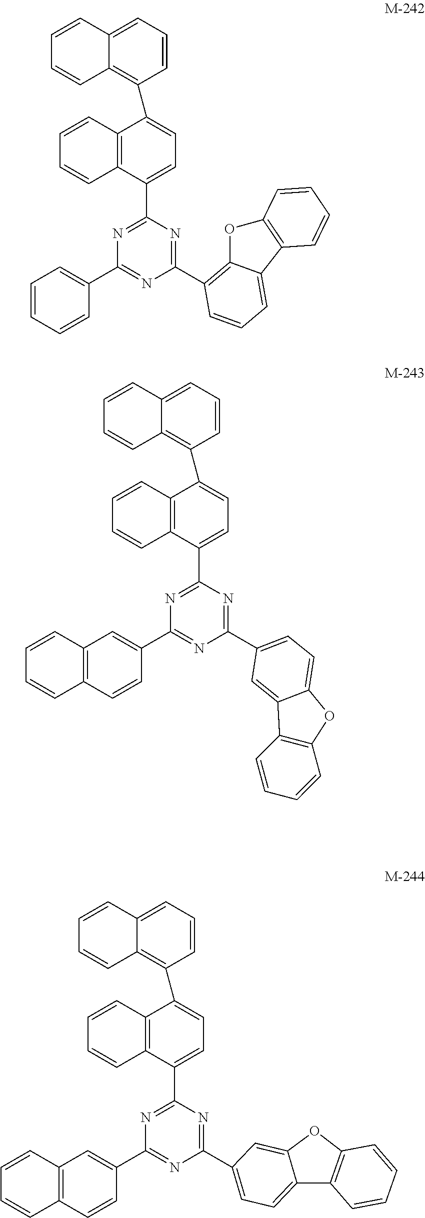 [SMILES: c1ccc(-c2nc(-c3ccc(-c4cccc5ccccc45)c4ccccc34)nc(-c3cccc4c3oc3ccccc34)n2)cc1 ; c1ccc2cc(-c3nc(-c4ccc5oc6ccccc6c5c4)nc(-c4ccc(-c5cccc6ccccc56)c5ccccc45)n3)ccc2c1 ; c1ccc2cc(-c3nc(-c4ccc5c(c4)oc4ccccc45)nc(-c4ccc(-c5cccc6ccccc56)c5ccccc45)n3)ccc2c1]

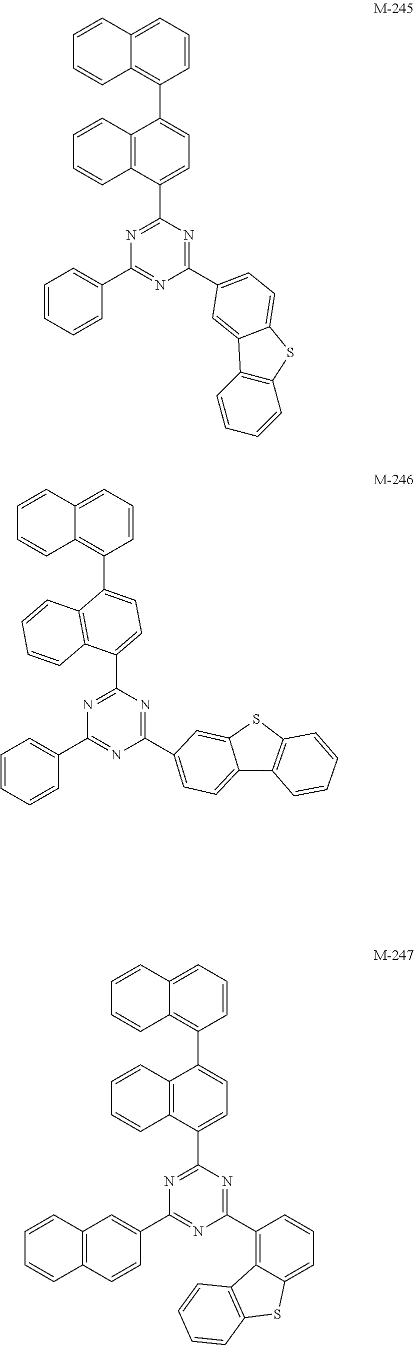 [SMILES: c1ccc(-c2nc(-c3ccc4sc5ccccc5c4c3)nc(-c3ccc(-c4cccc5ccccc45)c4ccccc34)n2)cc1 ; c1ccc(-c2nc(-c3ccc4c(c3)sc3ccccc34)nc(-c3ccc(-c4cccc5ccccc45)c4ccccc34)n2)cc1 ; c1ccc2cc(-c3nc(-c4ccc(-c5cccc6ccccc56)c5ccccc45)nc(-c4cccc5sc6ccccc6c45)n3)ccc2c1]

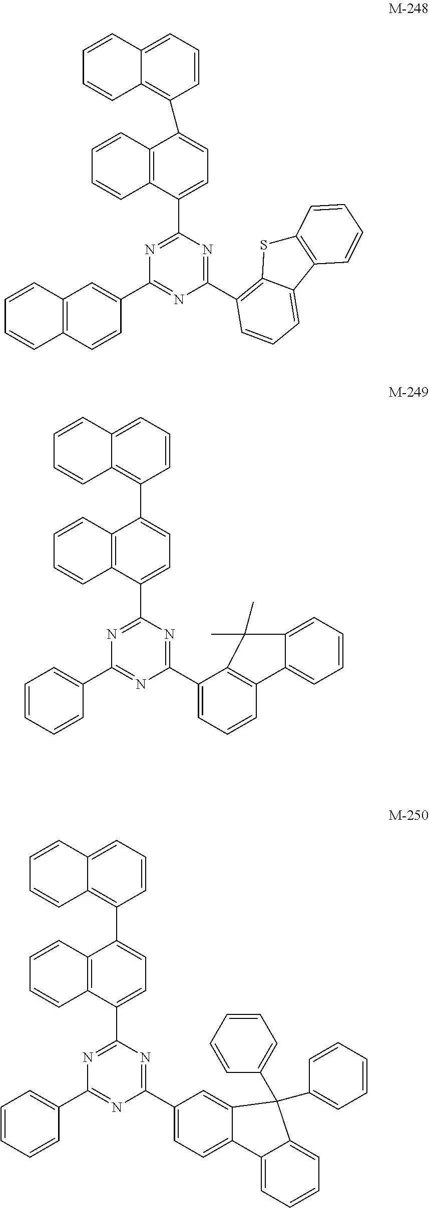 [SMILES: c1ccc2cc(-c3nc(-c4ccc(-c5cccc6ccccc56)c5ccccc45)nc(-c4cccc5c4sc4ccccc45)n3)ccc2c1 ; CC1(C)c2ccccc2-c2cccc(-c3nc(-c4ccccc4)nc(-c4ccc(-c5cccc6ccccc56)c5ccccc45)n3)c21 ; c1ccc(-c2nc(-c3ccc4c(c3)C(c3ccccc3)(c3ccccc3)c3ccccc3-4)nc(-c3ccc(-c4cccc5ccccc45)c4ccccc34)n2)cc1]

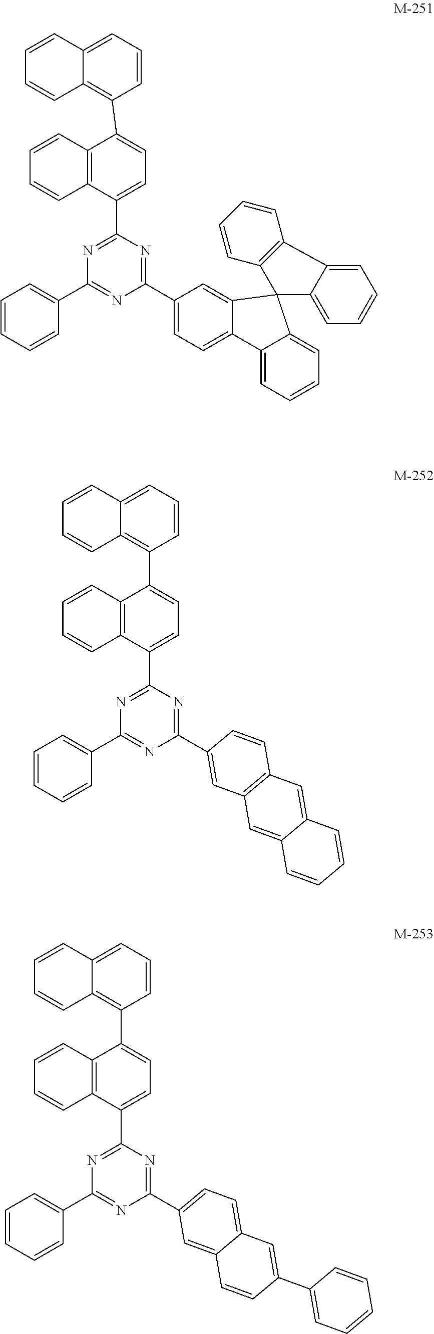 [SMILES: c1ccc(-c2nc(-c3ccc4c(c3)C3(c5ccccc5-c5ccccc53)c3ccccc3-4)nc(-c3ccc(-c4cccc5ccccc45)c4ccccc34)n2)cc1 ; c1ccc(-c2nc(-c3ccc4cc5ccccc5cc4c3)nc(-c3ccc(-c4cccc5ccccc45)c4ccccc34)n2)cc1 ; c1ccc(-c2ccc3cc(-c4nc(-c5ccccc5)nc(-c5ccc(-c6cccc7ccccc67)c6ccccc56)n4)ccc3c2)cc1]

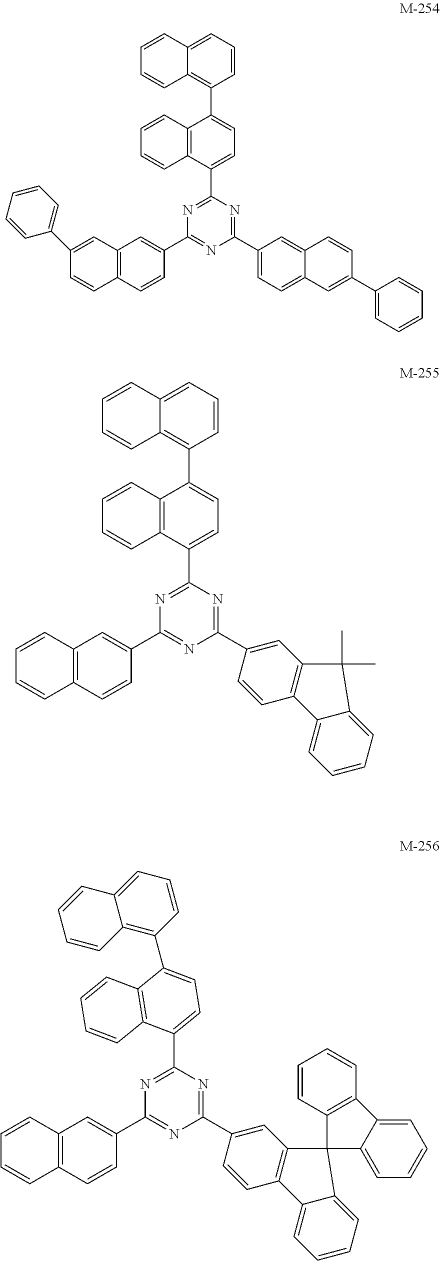 [SMILES: c1ccc(-c2ccc3cc(-c4nc(-c5ccc6ccc(-c7ccccc7)cc6c5)nc(-c5ccc(-c6cccc7ccccc67)c6ccccc56)n4)ccc3c2)cc1 ; CC1(C)c2ccccc2-c2ccc(-c3nc(-c4ccc5ccccc5c4)nc(-c4ccc(-c5cccc6ccccc56)c5ccccc45)n3)cc21 ; c1ccc2c(c1)-c1ccccc1C21c2ccccc2-c2ccc(-c3nc(-c4ccc5ccccc5c4)nc(-c4ccc(-c5cccc6ccccc56)c5ccccc45)n3)cc21]

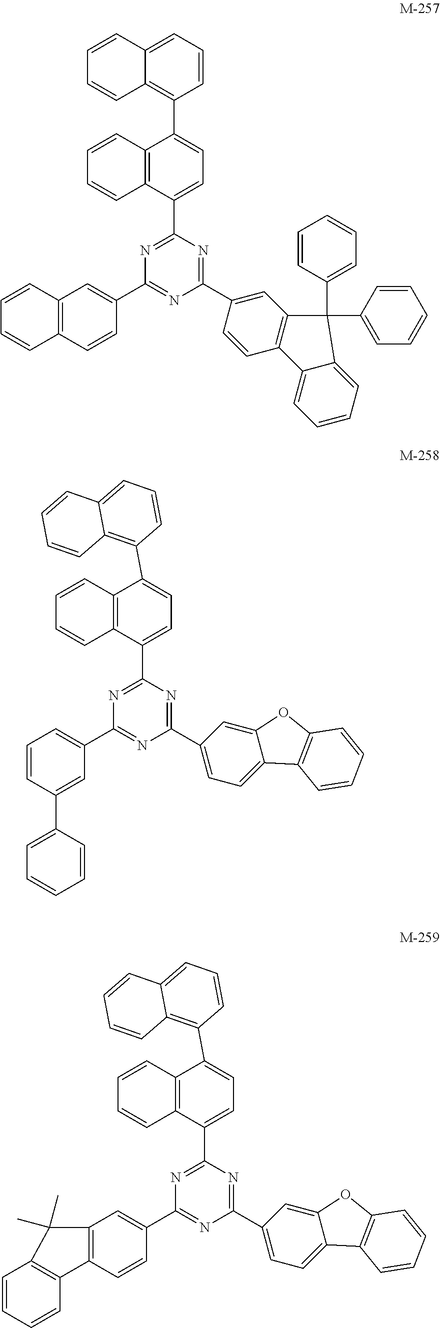 [SMILES: c1ccc(C2(c3ccccc3)c3ccccc3-c3ccc(-c4nc(-c5ccc6ccccc6c5)nc(-c5ccc(-c6cccc7ccccc67)c6ccccc56)n4)cc32)cc1 ; c1ccc(-c2cccc(-c3nc(-c4ccc5c(c4)oc4ccccc45)nc(-c4ccc(-c5cccc6ccccc56)c5ccccc45)n3)c2)cc1 ; CC1(C)c2ccccc2-c2ccc(-c3nc(-c4ccc5c(c4)oc4ccccc45)nc(-c4ccc(-c5cccc6ccccc56)c5ccccc45)n3)cc21]

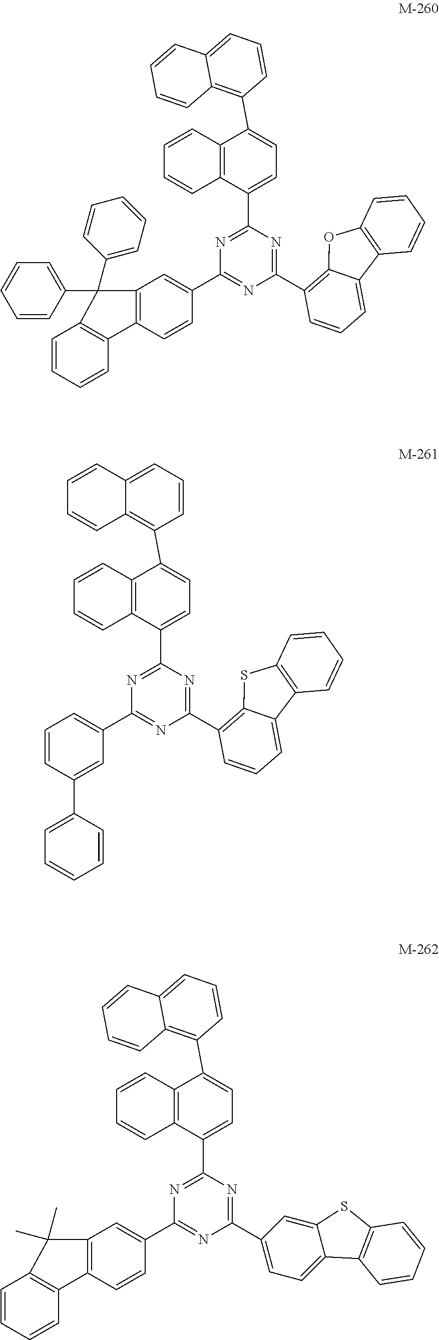 [SMILES: c1ccc(C2(c3ccccc3)c3ccccc3-c3ccc(-c4nc(-c5ccc(-c6cccc7ccccc67)c6ccccc56)nc(-c5cccc6c5oc5ccccc56)n4)cc32)cc1 ; c1ccc(-c2cccc(-c3nc(-c4ccc(-c5cccc6ccccc56)c5ccccc45)nc(-c4cccc5c4sc4ccccc45)n3)c2)cc1 ; CC1(C)c2ccccc2-c2ccc(-c3nc(-c4ccc5c(c4)sc4ccccc45)nc(-c4ccc(-c5cccc6ccccc56)c5ccccc45)n3)cc21]

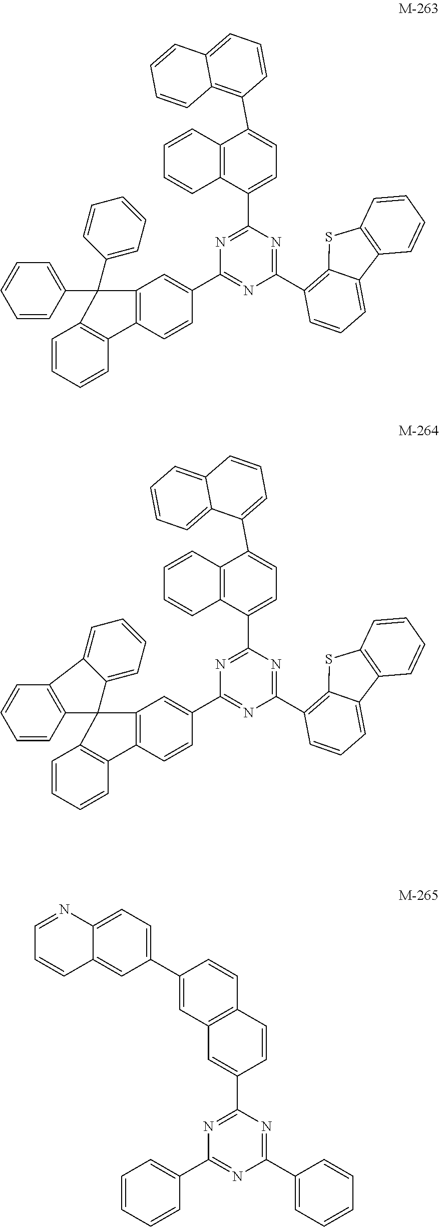 [SMILES: c1ccc(C2(c3ccccc3)c3ccccc3-c3ccc(-c4nc(-c5ccc(-c6cccc7ccccc67)c6ccccc56)nc(-c5cccc6c5sc5ccccc56)n4)cc32)cc1 ; c1ccc2c(c1)-c1ccccc1C21c2ccccc2-c2ccc(-c3nc(-c4ccc(-c5cccc6ccccc56)c5ccccc45)nc(-c4cccc5c4sc4ccccc45)n3)cc21 ; c1ccc(-c2nc(-c3ccccc3)nc(-c3ccc4ccc(-c5ccc6ncccc6c5)cc4c3)n2)cc1]

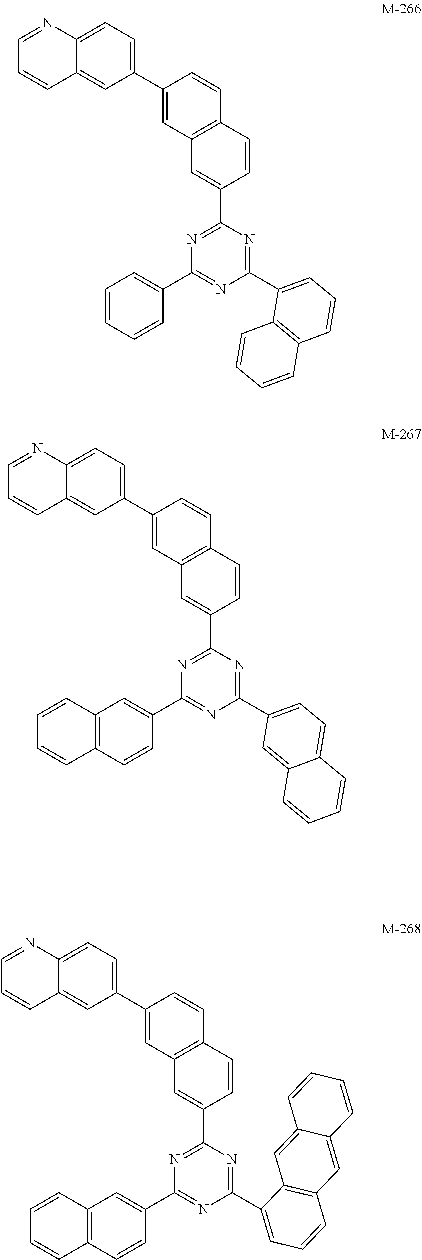 [SMILES: c1ccc(-c2nc(-c3ccc4ccc(-c5ccc6ncccc6c5)cc4c3)nc(-c3cccc4ccccc34)n2)cc1 ; c1ccc2cc(-c3nc(-c4ccc5ccccc5c4)nc(-c4ccc5ccc(-c6ccc7ncccc7c6)cc5c4)n3)ccc2c1 ; c1ccc2cc(-c3nc(-c4ccc5ccc(-c6ccc7ncccc7c6)cc5c4)nc(-c4cccc5cc6ccccc6cc45)n3)ccc2c1]

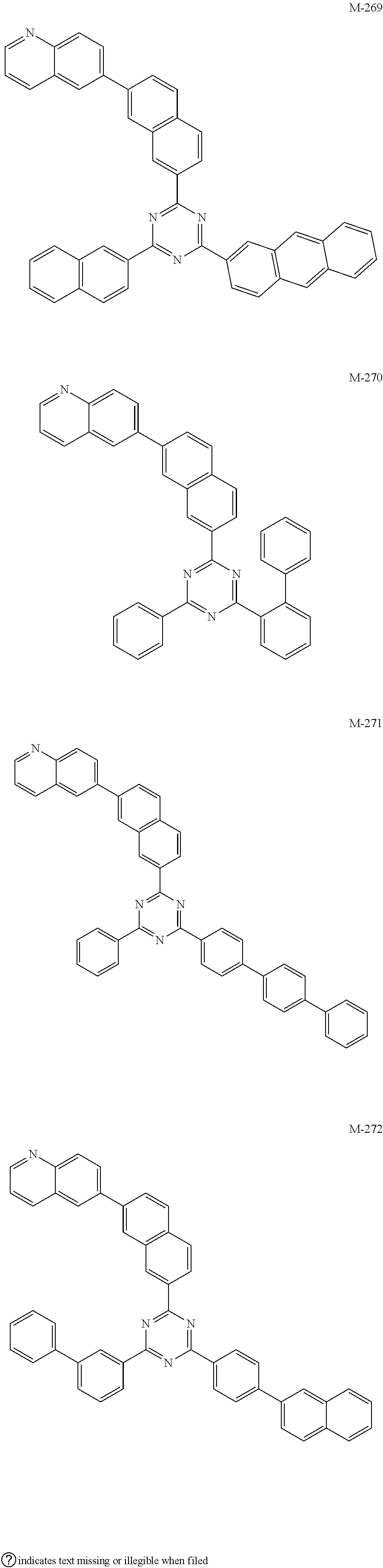 [SMILES: c1ccc2cc(-c3nc(-c4ccc5ccc(-c6ccc7ncccc7c6)cc5c4)nc(-c4ccc5cc6ccccc6cc5c4)n3)ccc2c1 ; c1ccc(-c2nc(-c3ccc4ccc(-c5ccc6ncccc6c5)cc4c3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6c5)n4)cc3)cc2)cc1 ; c1ccc(-c2cccc(-c3nc(-c4ccc(-c5ccc6ccccc6c5)cc4)nc(-c4ccc5ccc(-c6ccc7ncccc7c6)cc5c4)n3)c2)cc1]

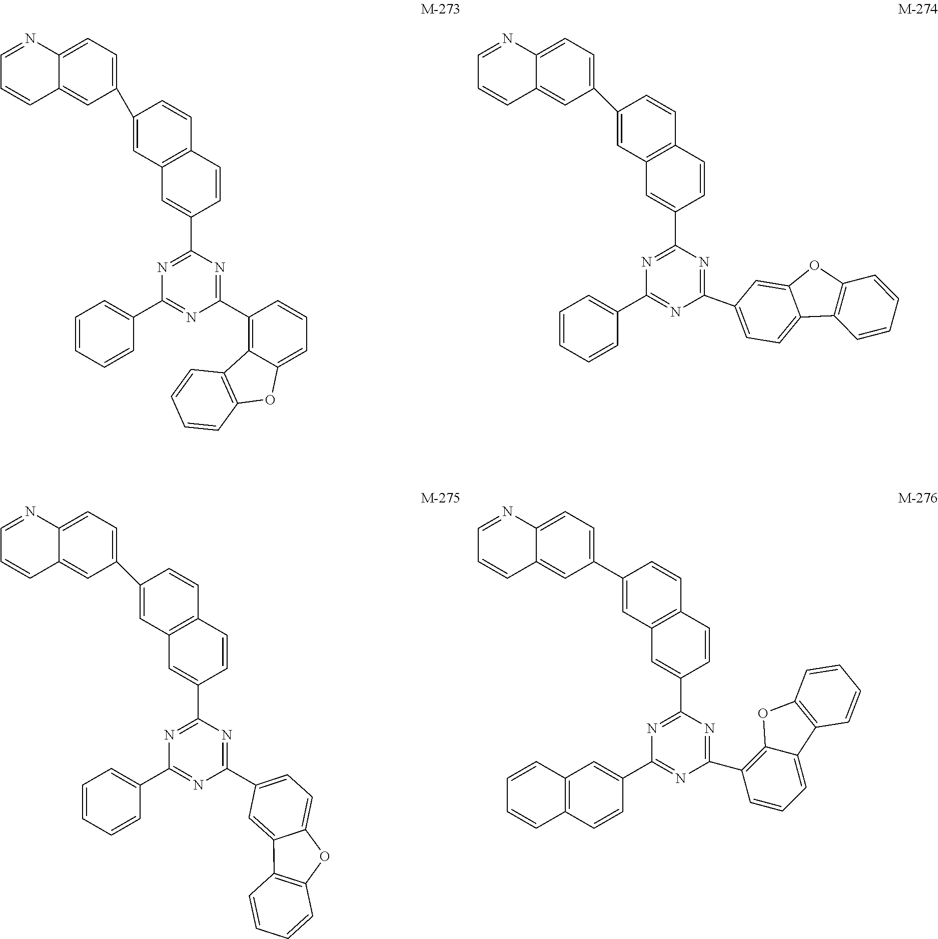 [SMILES: c1ccc(-c2nc(-c3ccc4ccc(-c5ccc6ncccc6c5)cc4c3)nc(-c3cccc4oc5ccccc5c34)n2)cc1 ; c1ccc(-c2nc(-c3ccc4ccc(-c5ccc6ncccc6c5)cc4c3)nc(-c3ccc4c(c3)oc3ccccc34)n2)cc1 ; c1ccc(-c2nc(-c3ccc4ccc(-c5ccc6ncccc6c5)cc4c3)nc(-c3ccc4oc5ccccc5c4c3)n2)cc1 ; c1ccc2cc(-c3nc(-c4ccc5ccc(-c6ccc7ncccc7c6)cc5c4)nc(-c4cccc5c4oc4ccccc45)n3)ccc2c1]

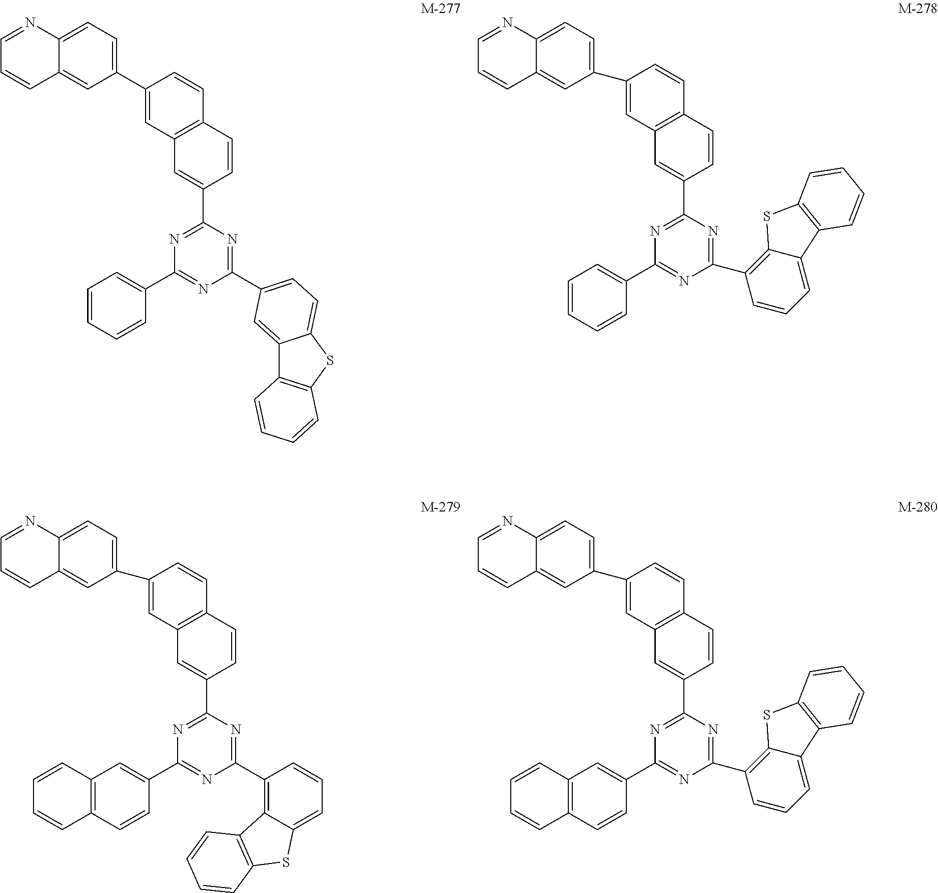 [SMILES: c1ccc(-c2nc(-c3ccc4ccc(-c5ccc6ncccc6c5)cc4c3)nc(-c3ccc4sc5ccccc5c4c3)n2)cc1 ; c1ccc(-c2nc(-c3ccc4ccc(-c5ccc6ncccc6c5)cc4c3)nc(-c3cccc4c3sc3ccccc34)n2)cc1 ; c1ccc2cc(-c3nc(-c4ccc5ccc(-c6ccc7ncccc7c6)cc5c4)nc(-c4cccc5sc6ccccc6c45)n3)ccc2c1 ; c1ccc2cc(-c3nc(-c4ccc5ccc(-c6ccc7ncccc7c6)cc5c4)nc(-c4cccc5c4sc4ccccc45)n3)ccc2c1]

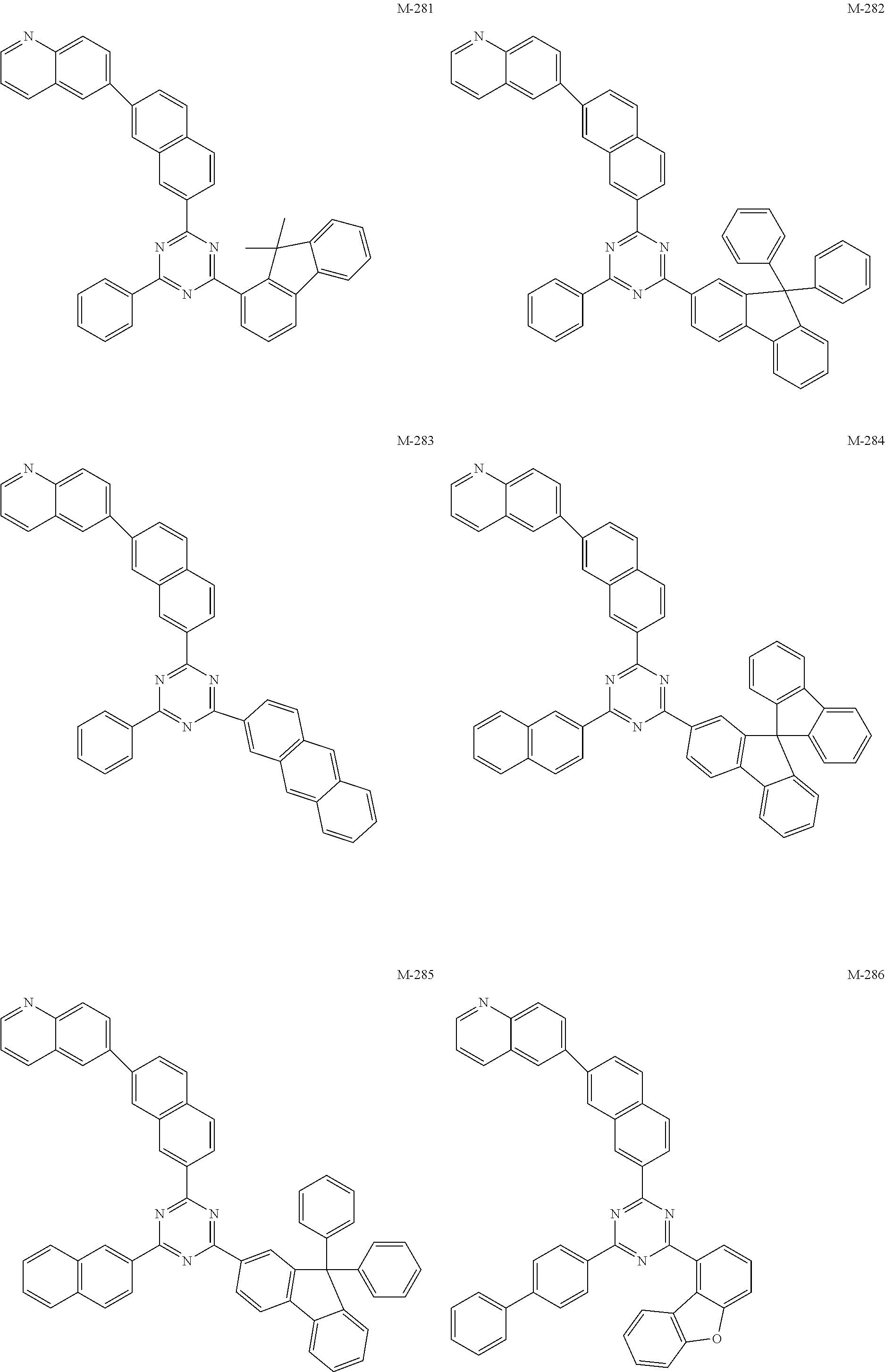

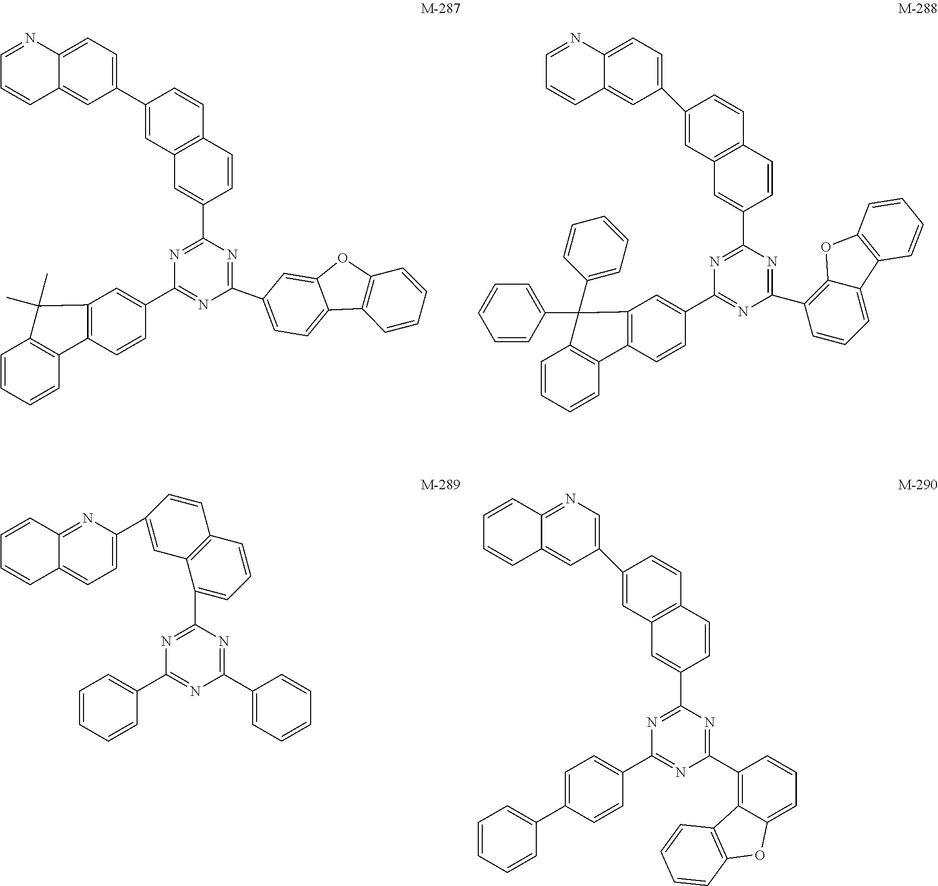 [SMILES: CC1(C)c2ccccc2-c2ccc(-c3nc(-c4ccc5ccc(-c6ccc7ncccc7c6)cc5c4)nc(-c4ccc5c(c4)oc4ccccc45)n3)cc21 ; c1ccc(C2(c3ccccc3)c3ccccc3-c3ccc(-c4nc(-c5ccc6ccc(-c7ccc8ncccc8c7)cc6c5)nc(-c5cccc6c5oc5ccccc56)n4)cc32)cc1 ; c1ccc(-c2nc(-c3ccccc3)nc(-c3cccc4ccc(-c5ccc6ccccc6n5)cc34)n2)cc1 ; c1ccc(-c2ccc(-c3nc(-c4ccc5ccc(-c6cnc7ccccc7c6)cc5c4)nc(-c4cccc5oc6ccccc6c45)n3)cc2)cc1]

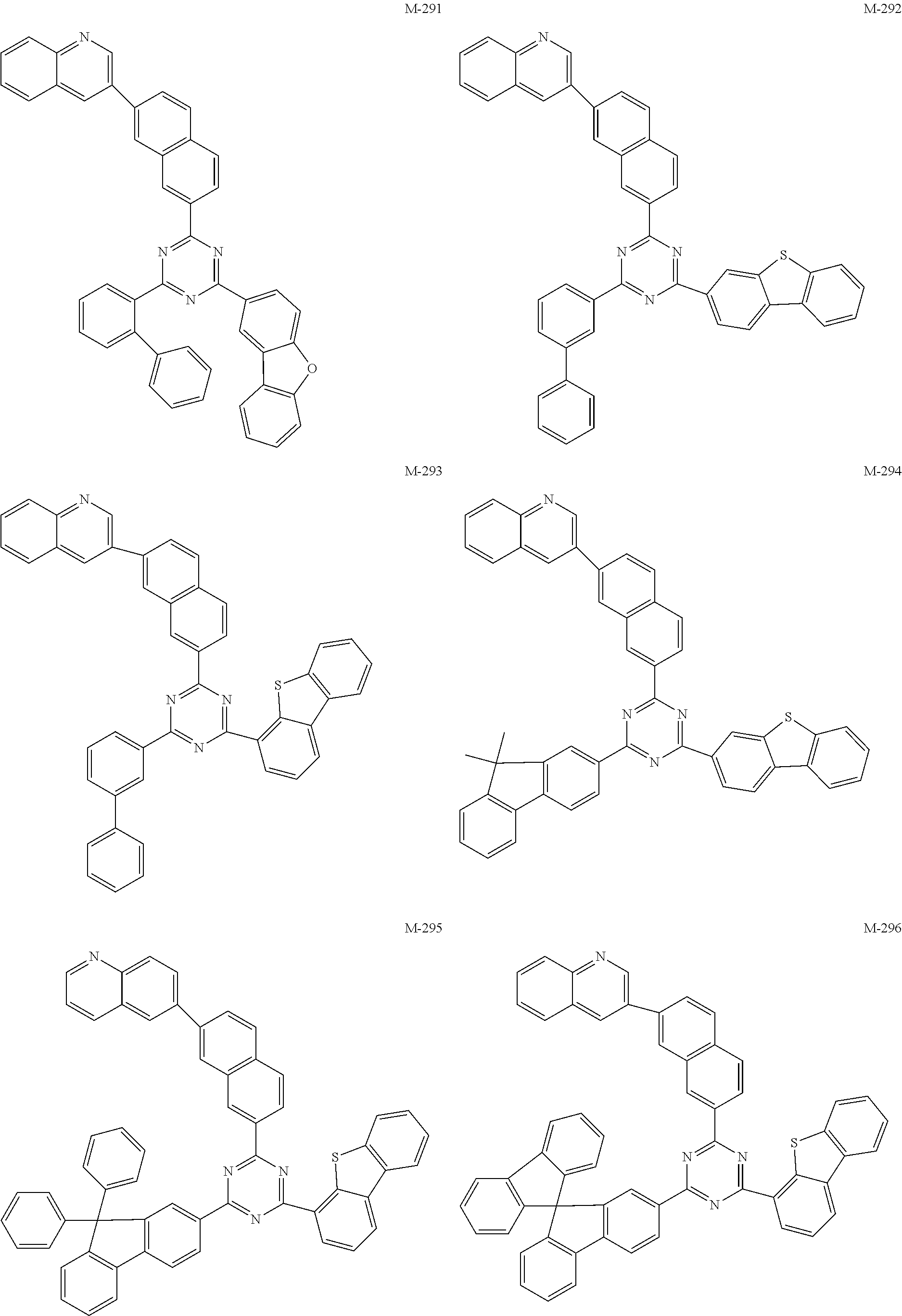

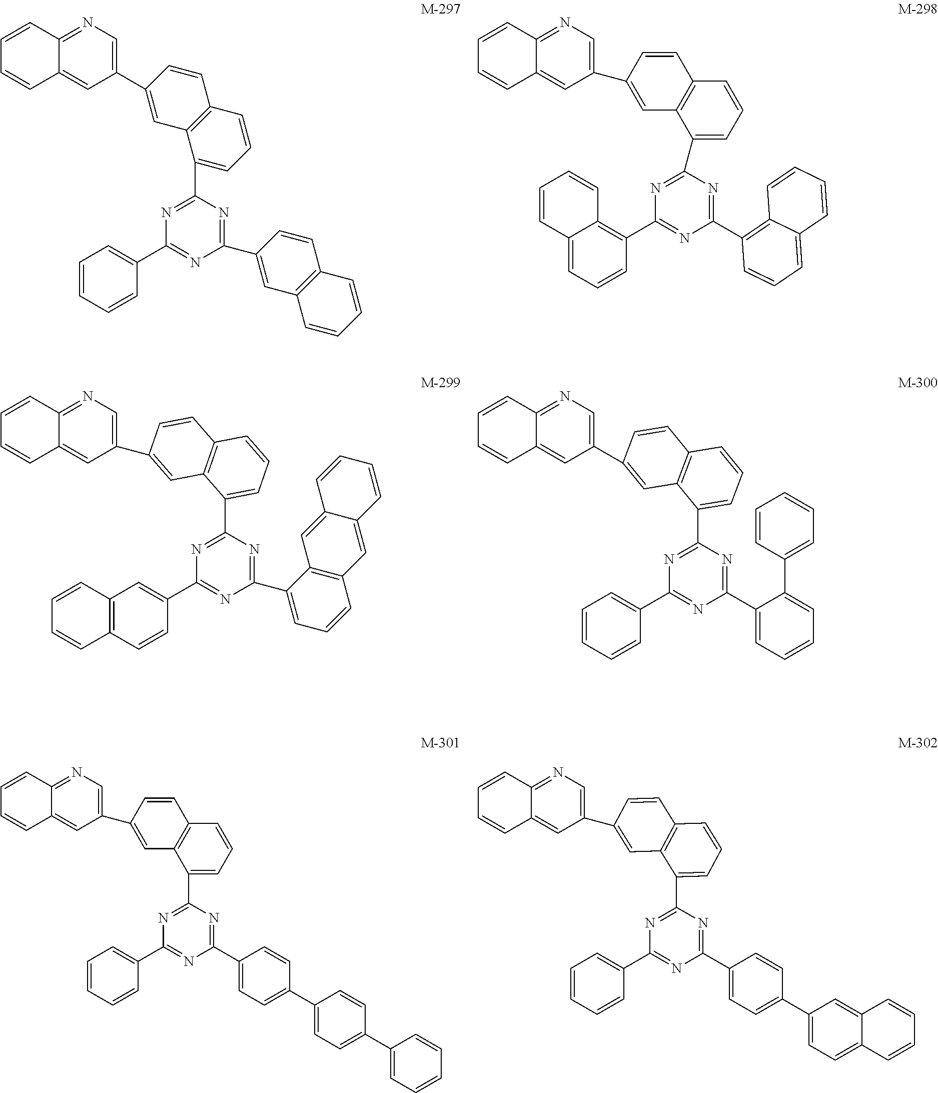 [SMILES: c1ccc(-c2nc(-c3ccc4ccccc4c3)nc(-c3cccc4ccc(-c5cnc6ccccc6c5)cc34)n2)cc1 ; c1ccc2ncc(-c3ccc4cccc(-c5nc(-c6cccc7ccccc67)nc(-c6cccc7ccccc67)n5)c4c3)cc2c1 ; c1ccc2cc(-c3nc(-c4cccc5ccc(-c6cnc7ccccc7c6)cc45)nc(-c4cccc5cc6ccccc6cc45)n3)ccc2c1 ; c1ccc(-c2nc(-c3ccccc3-c3ccccc3)nc(-c3cccc4ccc(-c5cnc6ccccc6c5)cc34)n2)cc1 ; c1ccc(-c2ccc(-c3ccc(-c4nc(-c5ccccc5)nc(-c5cccc6ccc(-c7cnc8ccccc8c7)cc56)n4)cc3)cc2)cc1 ; c1ccc(-c2nc(-c3ccc(-c4ccc5ccccc5c4)cc3)nc(-c3cccc4ccc(-c5cnc6ccccc6c5)cc34)n2)cc1]

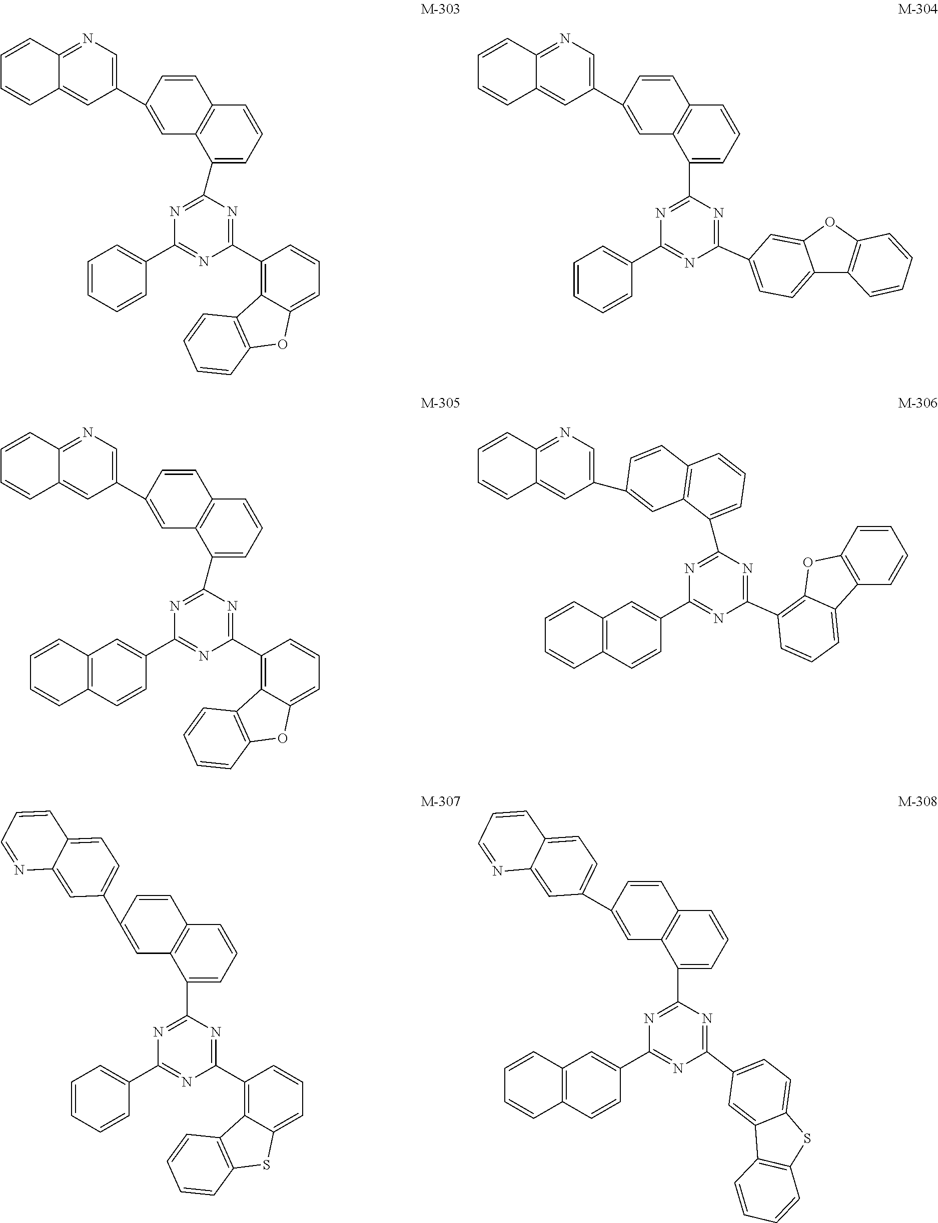 [SMILES: c1ccc(-c2nc(-c3cccc4ccc(-c5cnc6ccccc6c5)cc34)nc(-c3cccc4oc5ccccc5c34)n2)cc1 ; c1ccc(-c2nc(-c3ccc4c(c3)oc3ccccc34)nc(-c3cccc4ccc(-c5cnc6ccccc6c5)cc34)n2)cc1 ; c1ccc2cc(-c3nc(-c4cccc5ccc(-c6cnc7ccccc7c6)cc45)nc(-c4cccc5oc6ccccc6c45)n3)ccc2c1 ; c1ccc2cc(-c3nc(-c4cccc5ccc(-c6cnc7ccccc7c6)cc45)nc(-c4cccc5c4oc4ccccc45)n3)ccc2c1 ; c1ccc(-c2nc(-c3cccc4ccc(-c5ccc6cccnc6c5)cc34)nc(-c3cccc4sc5ccccc5c34)n2)cc1 ; c1ccc2cc(-c3nc(-c4ccc5sc6ccccc6c5c4)nc(-c4cccc5ccc(-c6ccc7cccnc7c6)cc45)n3)ccc2c1]

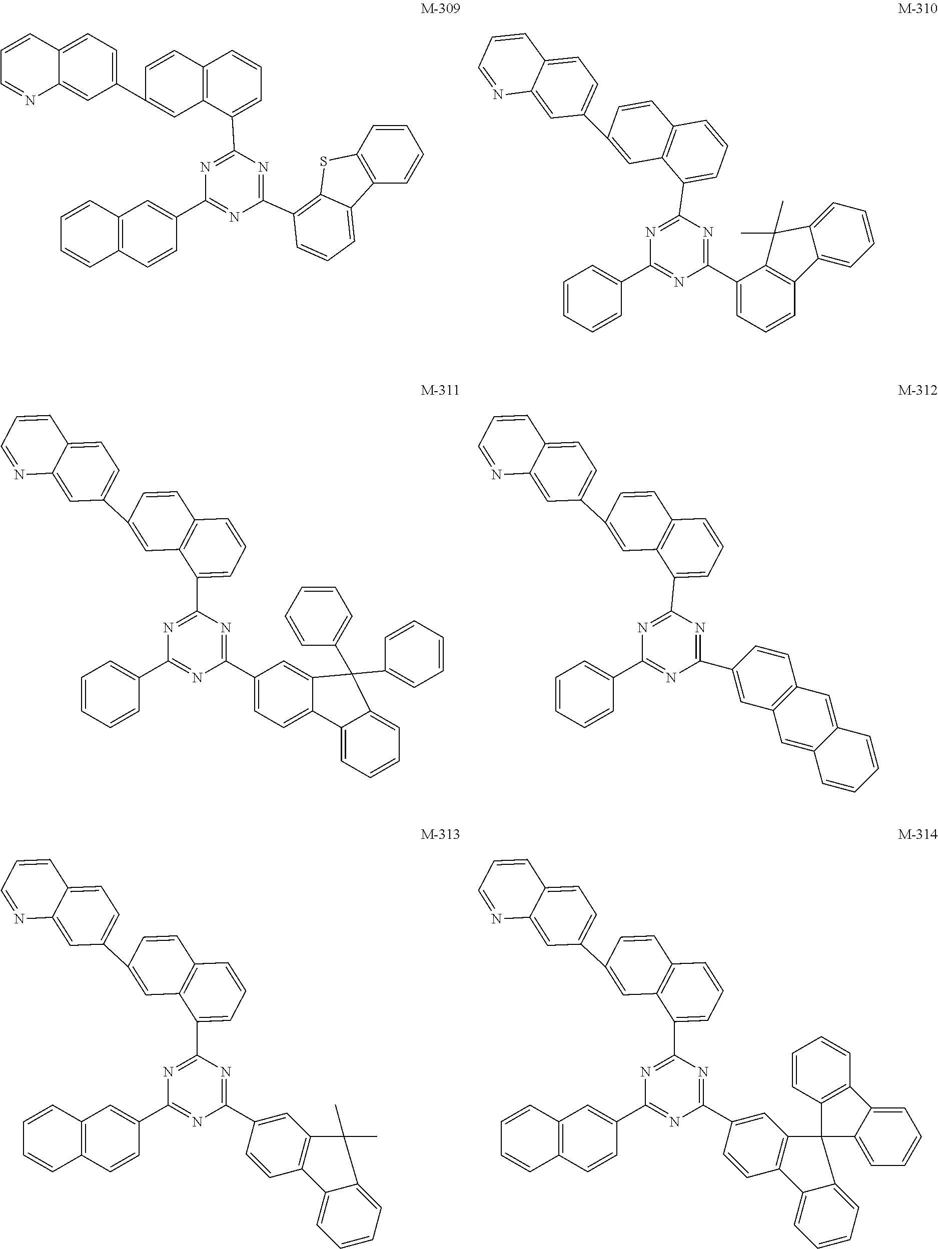

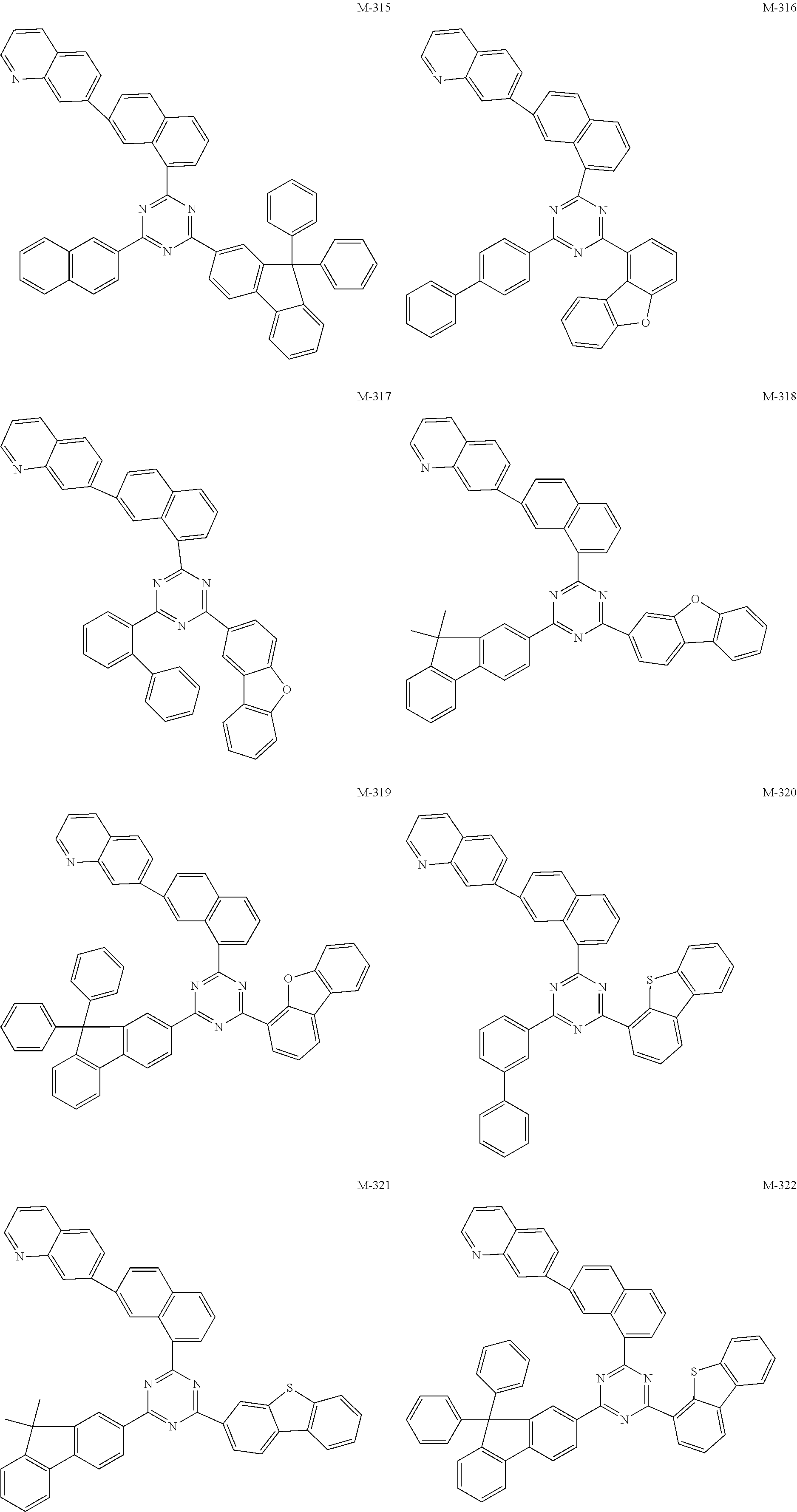

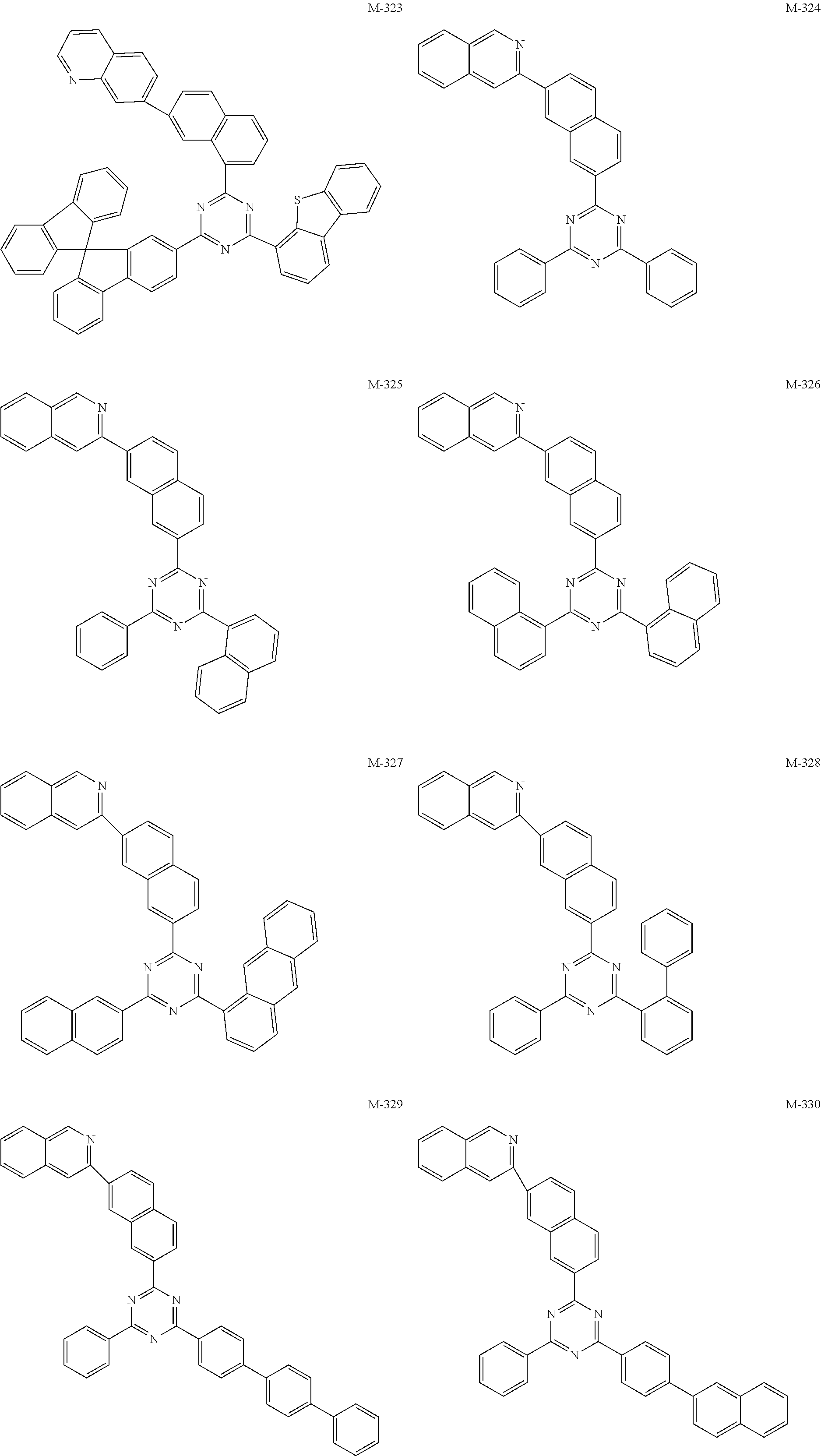

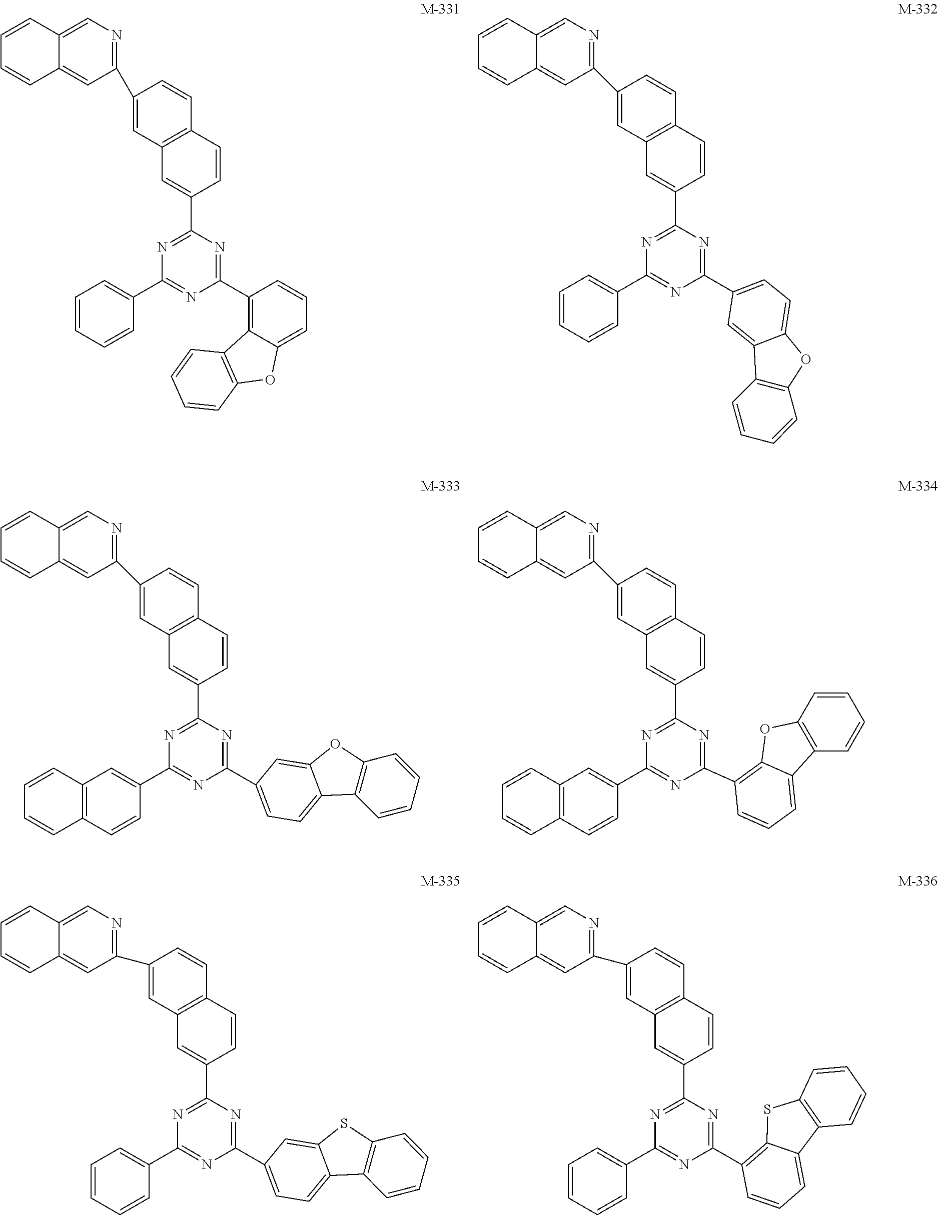 [SMILES: c1ccc(-c2nc(-c3ccc4ccc(-c5cc6ccccc6cn5)cc4c3)nc(-c3cccc4oc5ccccc5c34)n2)cc1 ; c1ccc(-c2nc(-c3ccc4ccc(-c5cc6ccccc6cn5)cc4c3)nc(-c3ccc4oc5ccccc5c4c3)n2)cc1 ; c1ccc2cc(-c3nc(-c4ccc5ccc(-c6cc7ccccc7cn6)cc5c4)nc(-c4ccc5c(c4)oc4ccccc45)n3)ccc2c1 ; c1ccc2cc(-c3nc(-c4ccc5ccc(-c6cc7ccccc7cn6)cc5c4)nc(-c4cccc5c4oc4ccccc45)n3)ccc2c1 ; c1ccc(-c2nc(-c3ccc4ccc(-c5cc6ccccc6cn5)cc4c3)nc(-c3ccc4c(c3)sc3ccccc34)n2)cc1 ; c1ccc(-c2nc(-c3ccc4ccc(-c5cc6ccccc6cn5)cc4c3)nc(-c3cccc4c3sc3ccccc34)n2)cc1]

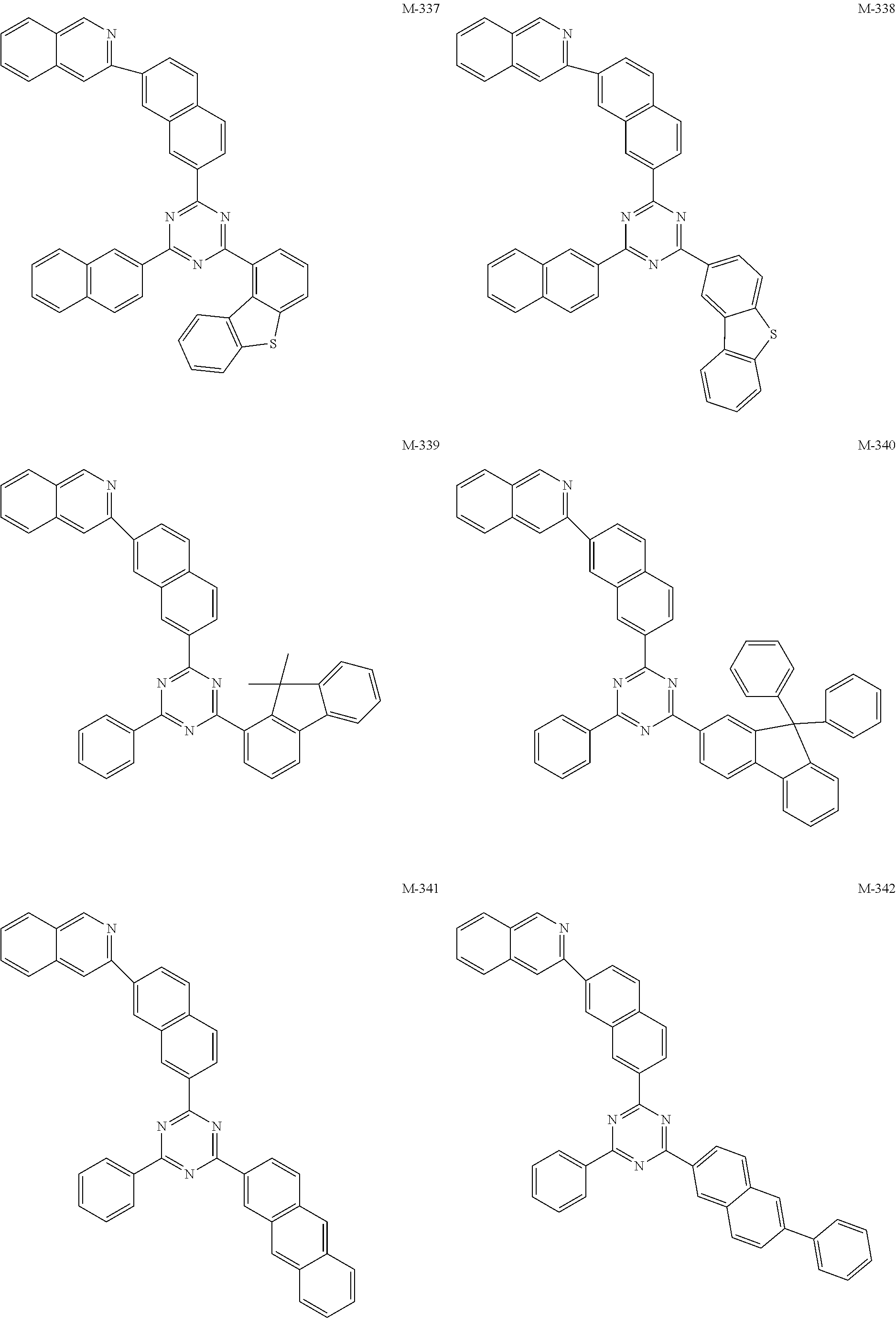

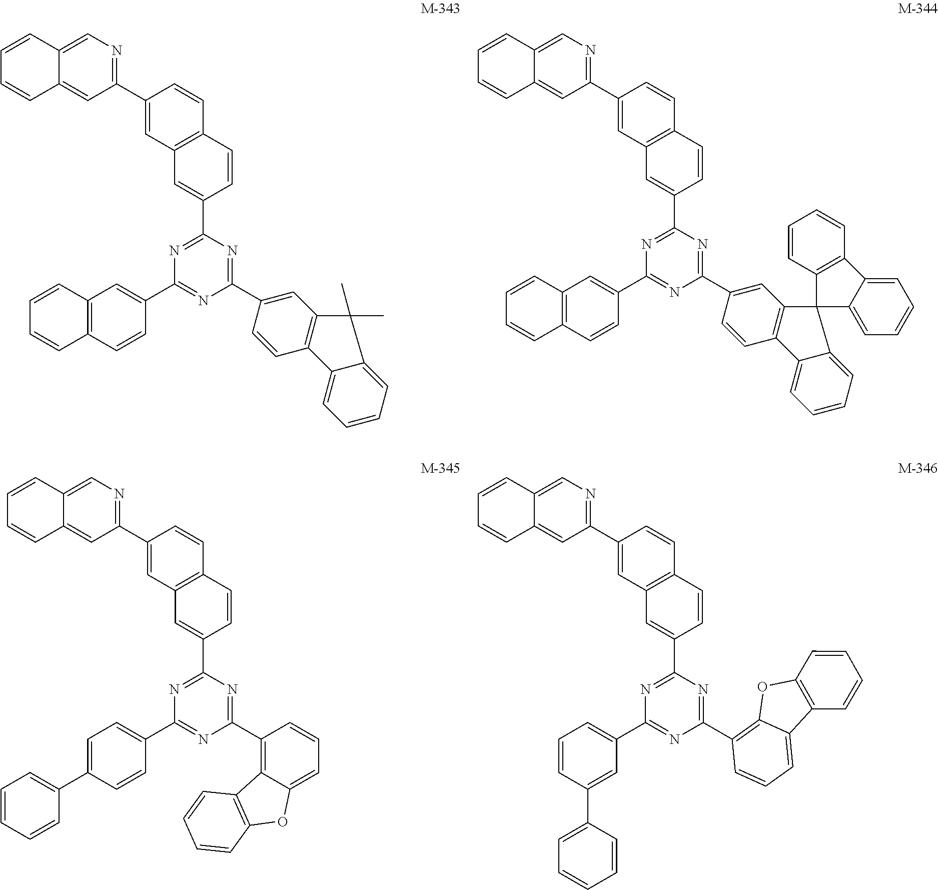 [SMILES: CC1(C)c2ccccc2-c2ccc(-c3nc(-c4ccc5ccccc5c4)nc(-c4ccc5ccc(-c6cc7ccccc7cn6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6cc7ccccc7cn6)cc5c4)n3)cc21 ; c1ccc(-c2ccc(-c3nc(-c4ccc5ccc(-c6cc7ccccc7cn6)cc5c4)nc(-c4cccc5oc6ccccc6c45)n3)cc2)cc1 ; c1ccc(-c2cccc(-c3nc(-c4ccc5ccc(-c6cc7ccccc7cn6)cc5c4)nc(-c4cccc5c4oc4ccccc45)n3)c2)cc1]

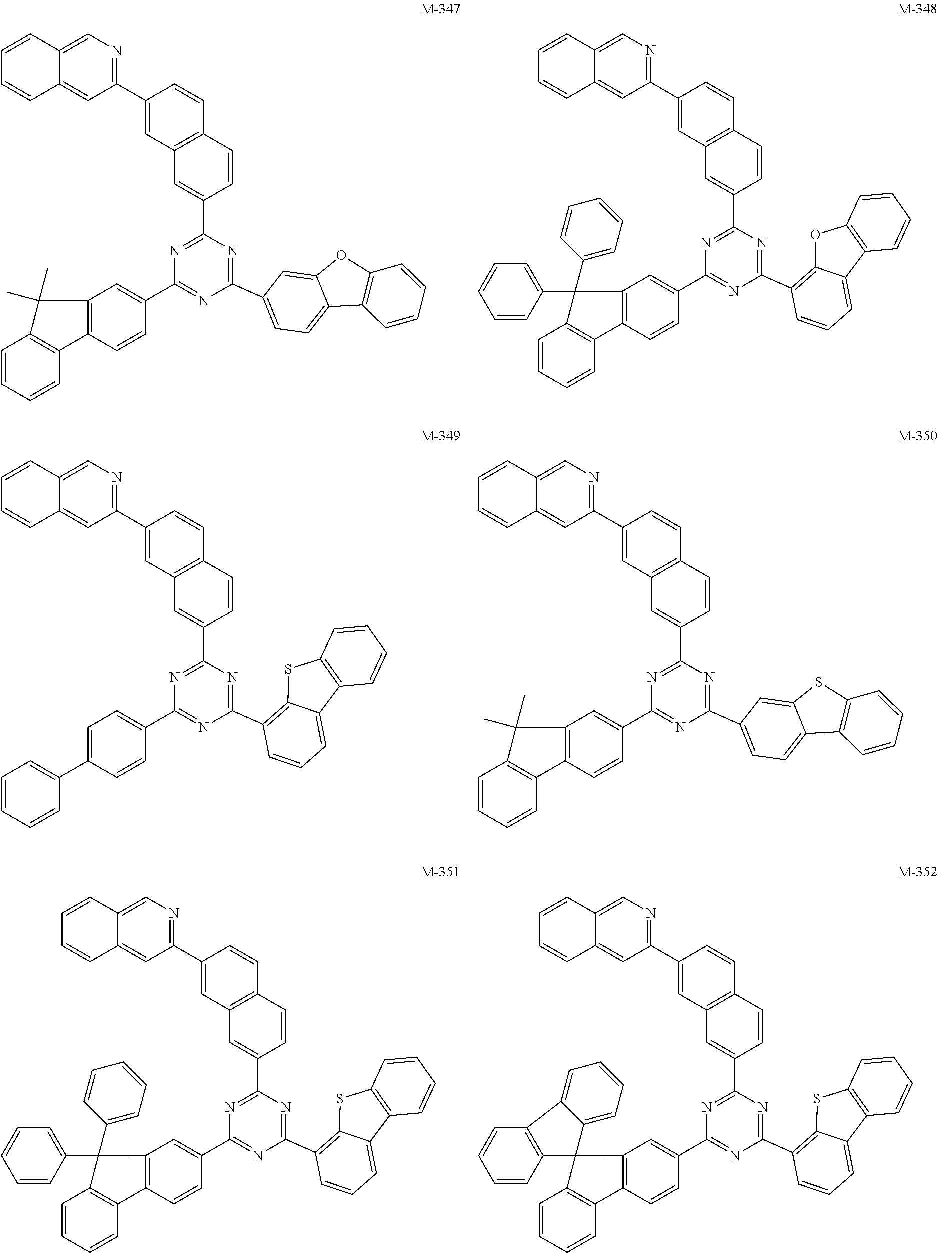

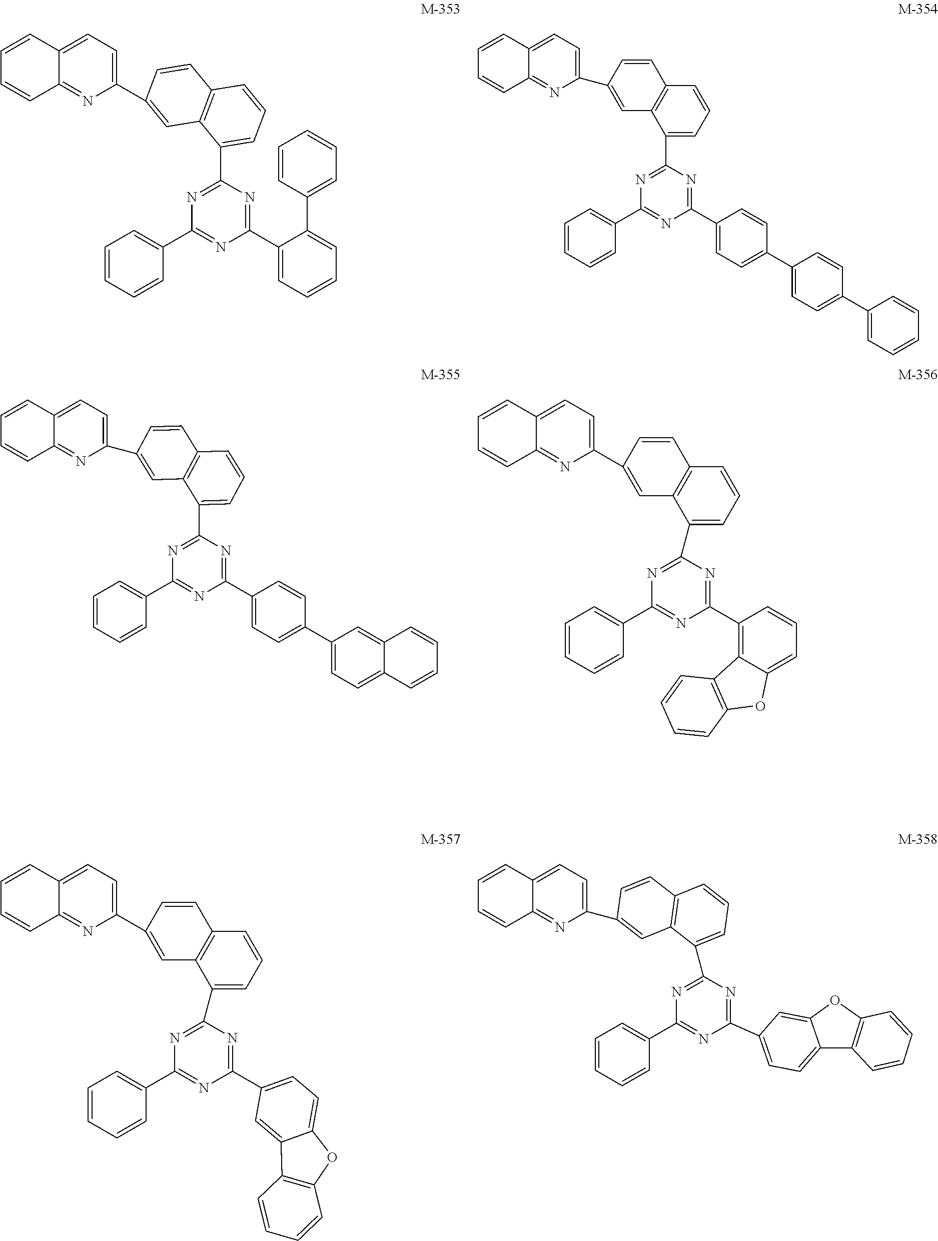 [SMILES: c1ccc(-c2nc(-c3ccccc3-c3ccccc3)nc(-c3cccc4ccc(-c5ccc6ccccc6n5)cc34)n2)cc1 ; c1ccc(-c2ccc(-c3ccc(-c4nc(-c5ccccc5)nc(-c5cccc6ccc(-c7ccc8ccccc8n7)cc56)n4)cc3)cc2)cc1 ; c1ccc(-c2nc(-c3ccc(-c4ccc5ccccc5c4)cc3)nc(-c3cccc4ccc(-c5ccc6ccccc6n5)cc34)n2)cc1 ; c1ccc(-c2nc(-c3cccc4ccc(-c5ccc6ccccc6n5)cc34)nc(-c3cccc4oc5ccccc5c34)n2)cc1 ; c1ccc(-c2nc(-c3ccc4oc5ccccc5c4c3)nc(-c3cccc4ccc(-c5ccc6ccccc6n5)cc34)n2)cc1 ; c1ccc(-c2nc(-c3ccc4c(c3)oc3ccccc34)nc(-c3cccc4ccc(-c5ccc6ccccc6n5)cc34)n2)cc1]

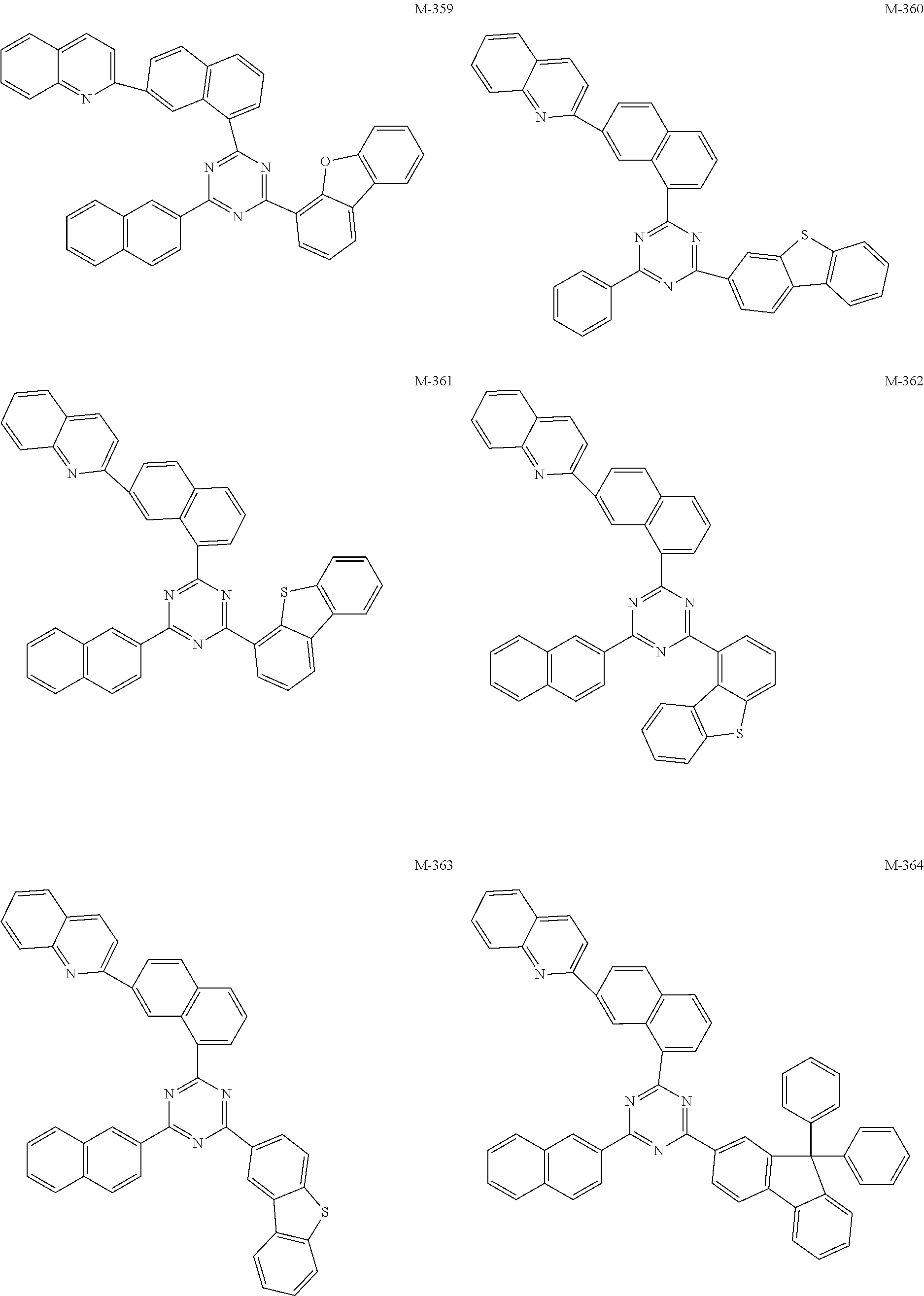

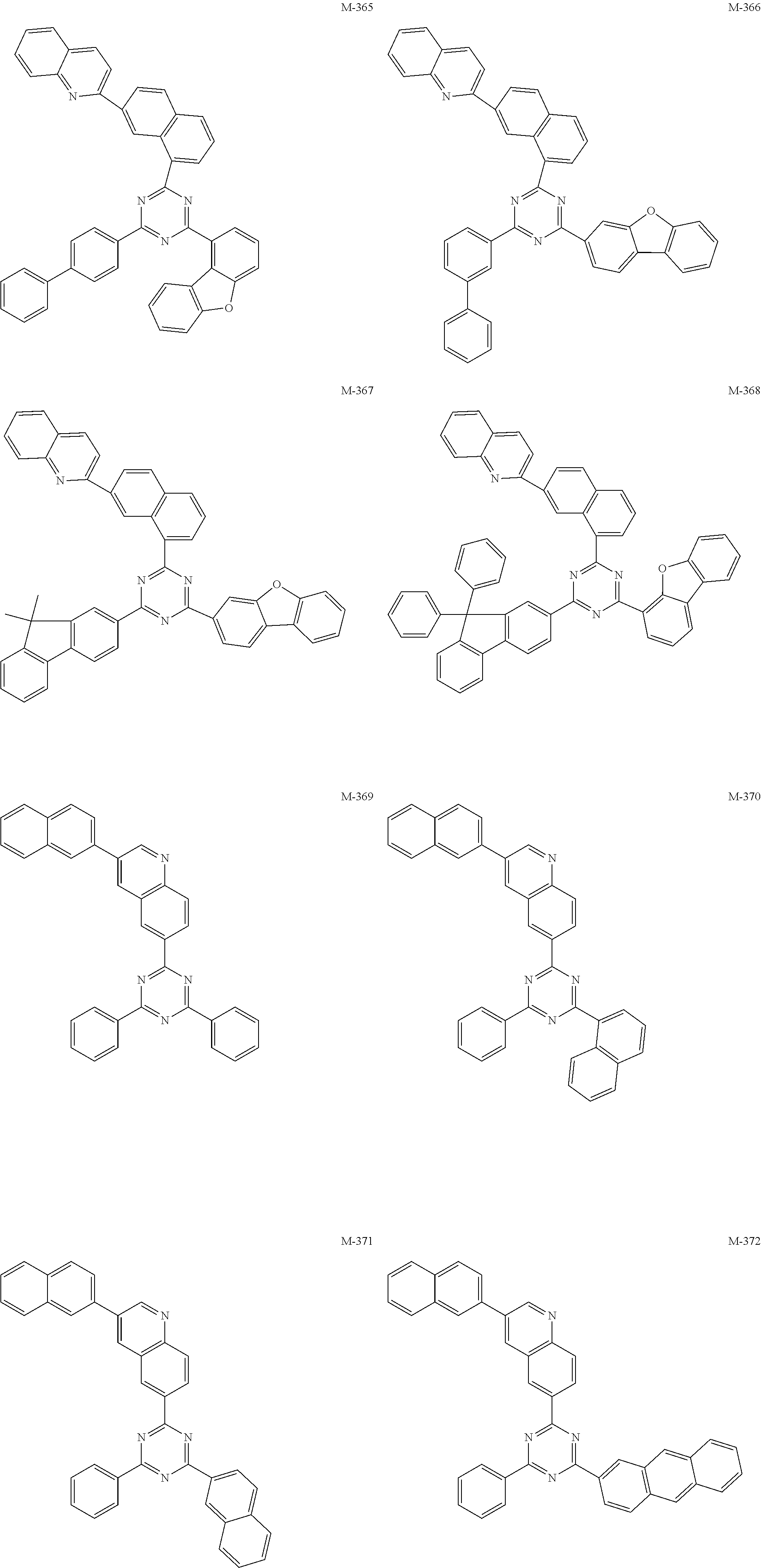

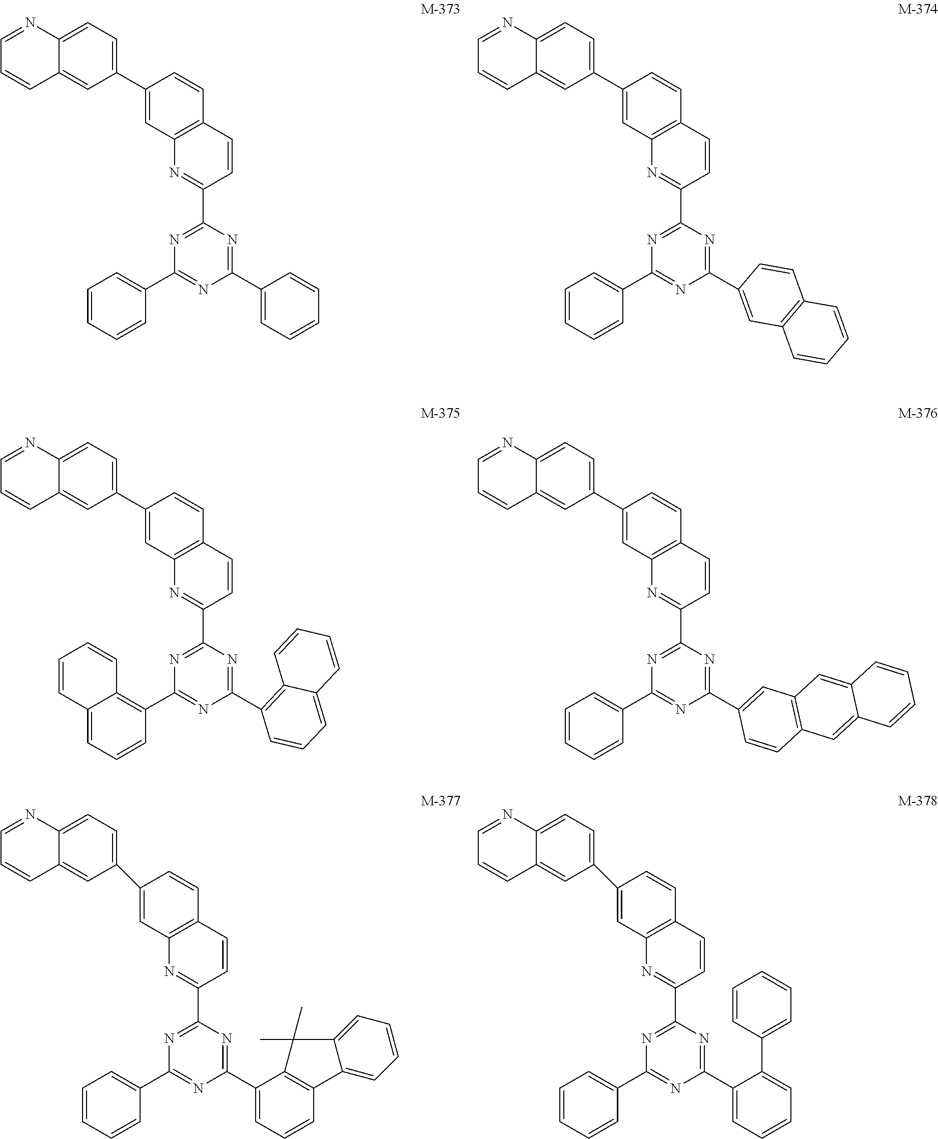 [SMILES: c1ccc(-c2nc(-c3ccccc3)nc(-c3ccc4ccc(-c5ccc6ncccc6c5)cc4n3)n2)cc1 ; c1ccc(-c2nc(-c3ccc4ccccc4c3)nc(-c3ccc4ccc(-c5ccc6ncccc6c5)cc4n3)n2)cc1 ; c1cnc2ccc(-c3ccc4ccc(-c5nc(-c6cccc7ccccc67)nc(-c6cccc7ccccc67)n5)nc4c3)cc2c1 ; c1ccc(-c2nc(-c3ccc4cc5ccccc5cc4c3)nc(-c3ccc4ccc(-c5ccc6ncccc6c5)cc4n3)n2)cc1 ; CC1(C)c2ccccc2-c2cccc(-c3nc(-c4ccccc4)nc(-c4ccc5ccc(-c6ccc7ncccc7c6)cc5n4)n3)c21 ; c1ccc(-c2nc(-c3ccc4ccc(-c5ccc6ncccc6c5)cc4n3)nc(-c3ccccc3-c3ccccc3)n2)cc1]

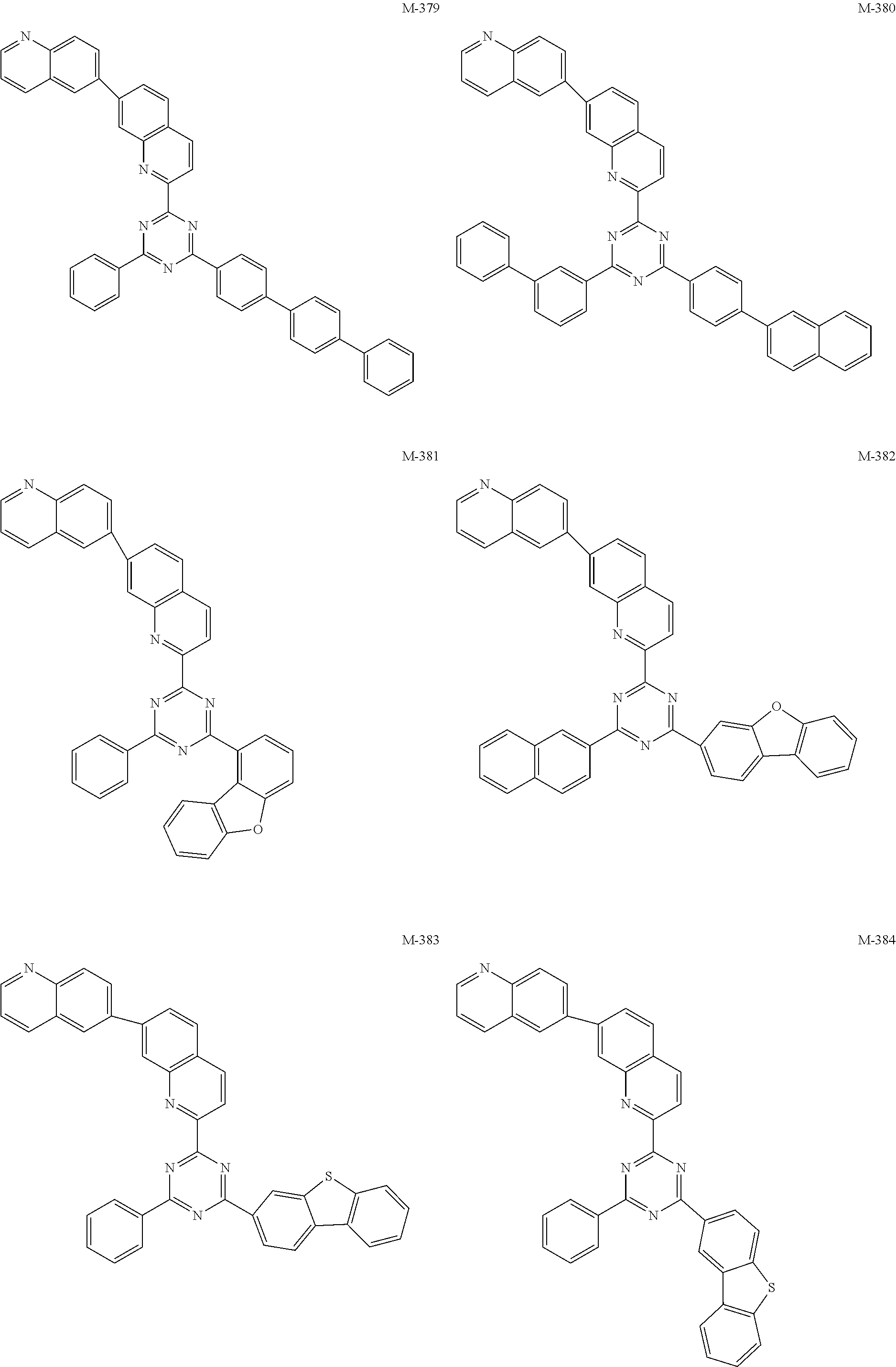

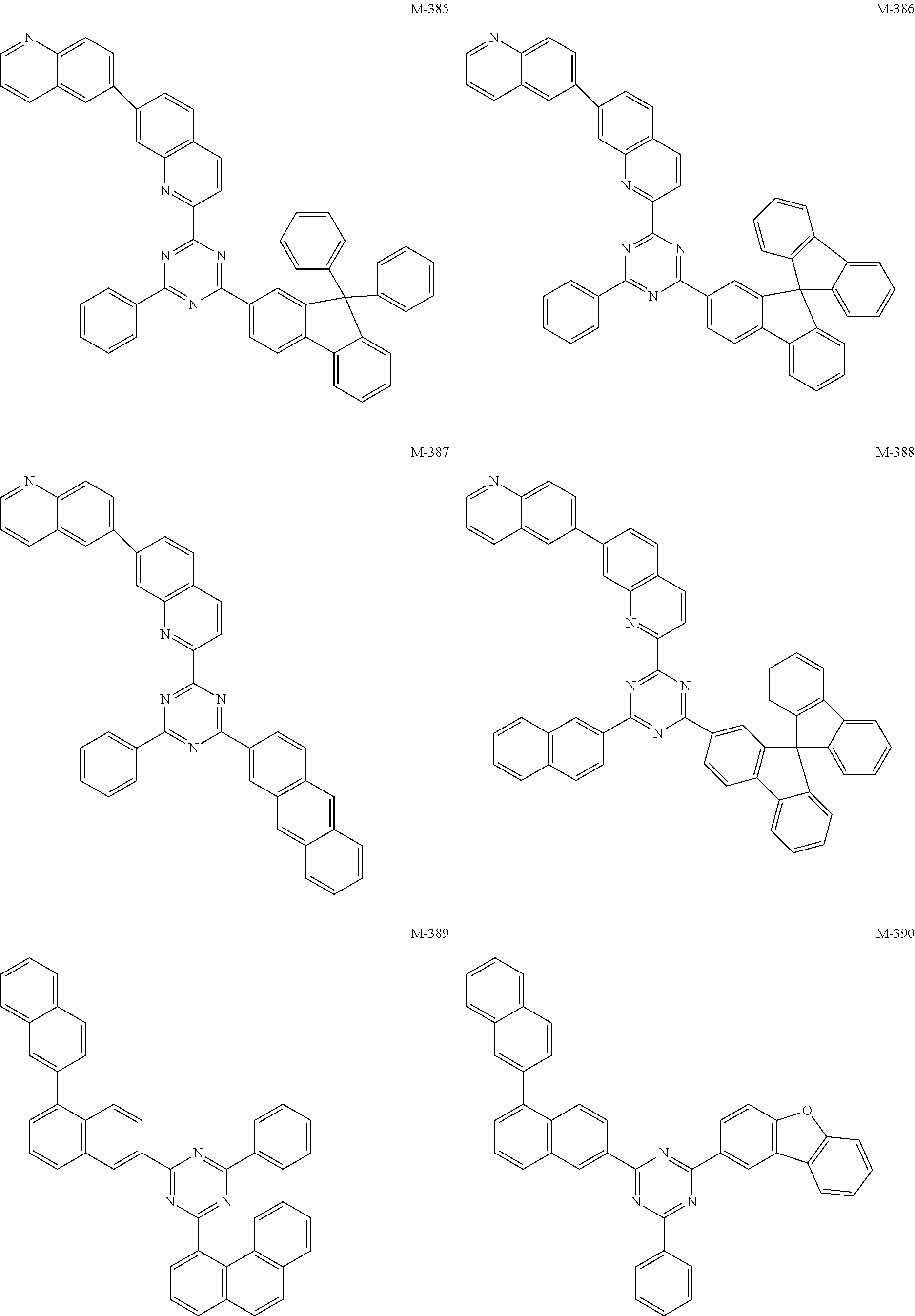

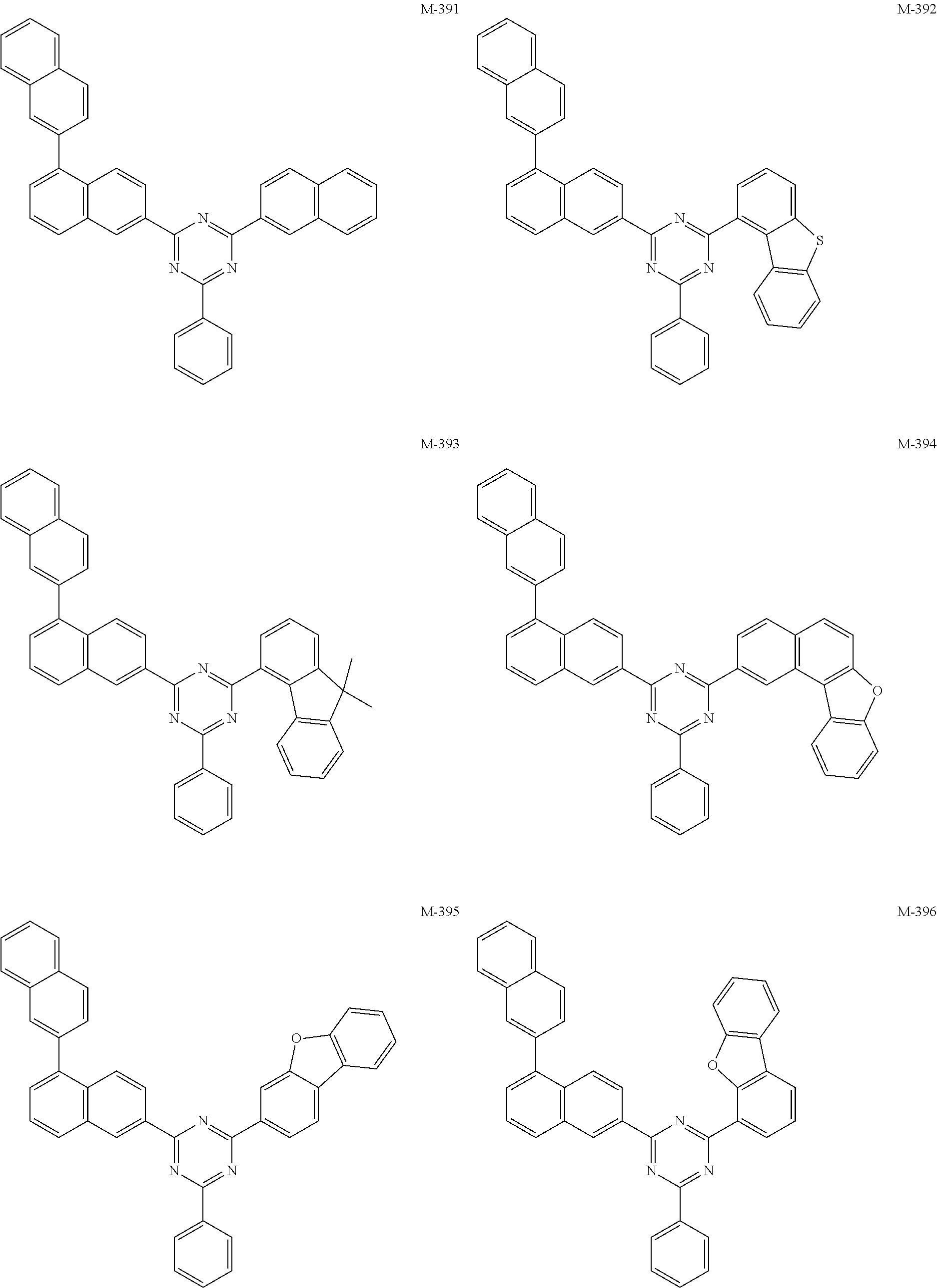 [SMILES: c1ccc(-c2nc(-c3ccc4ccccc4c3)nc(-c3ccc4c(-c5ccc6ccccc6c5)cccc4c3)n2)cc1 ; c1ccc(-c2nc(-c3ccc4c(-c5ccc6ccccc6c5)cccc4c3)nc(-c3cccc4sc5ccccc5c34)n2)cc1 ; CC1(C)c2ccccc2-c2c(-c3nc(-c4ccccc4)nc(-c4ccc5c(-c6ccc7ccccc7c6)cccc5c4)n3)cccc21 ; c1ccc(-c2nc(-c3ccc4c(-c5ccc6ccccc6c5)cccc4c3)nc(-c3ccc4ccc5oc6ccccc6c5c4c3)n2)cc1 ; c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4c(-c5ccc6ccccc6c5)cccc4c3)nc(-c3cccc4c3oc3ccccc34)n2)cc1]

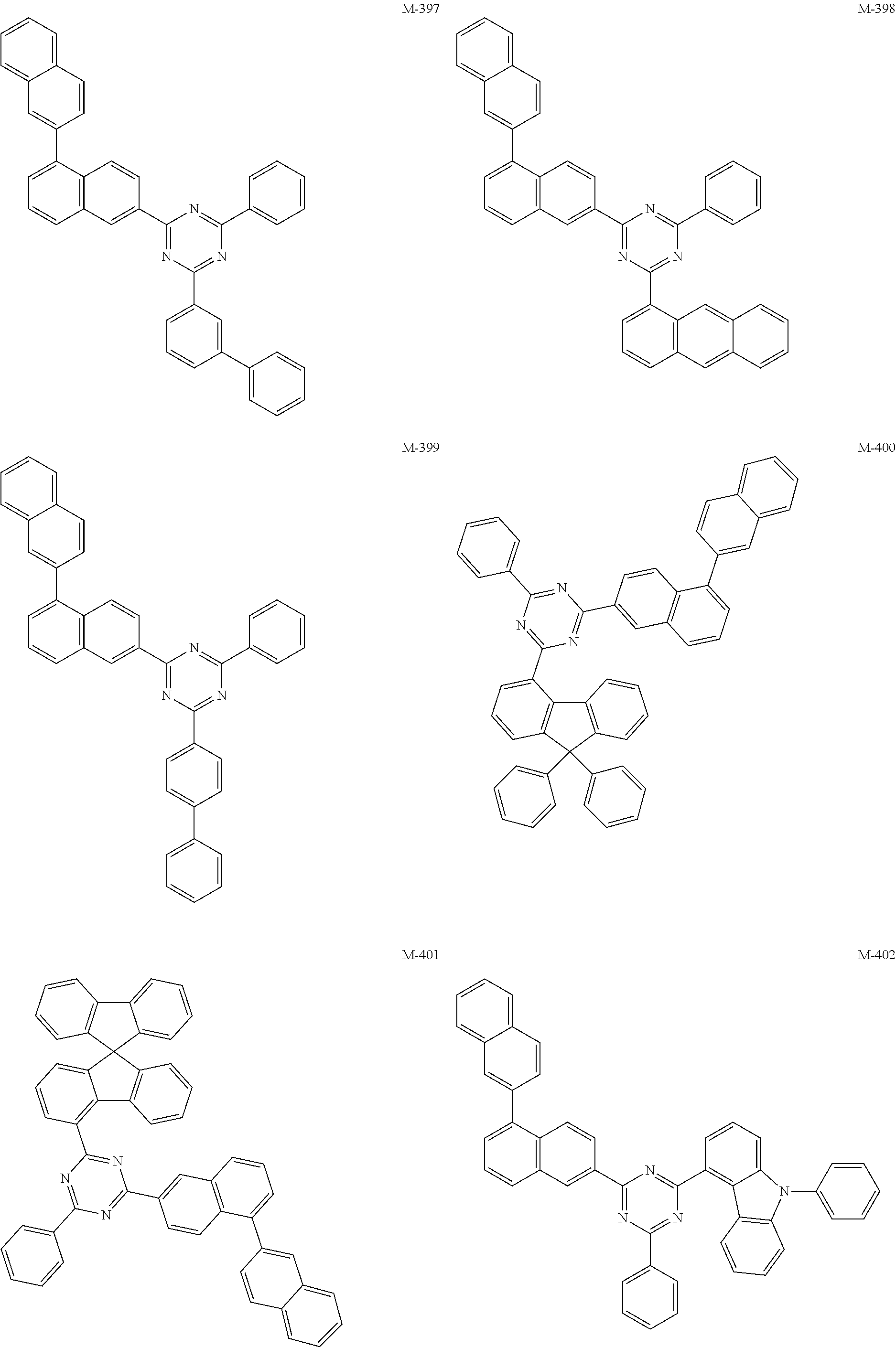

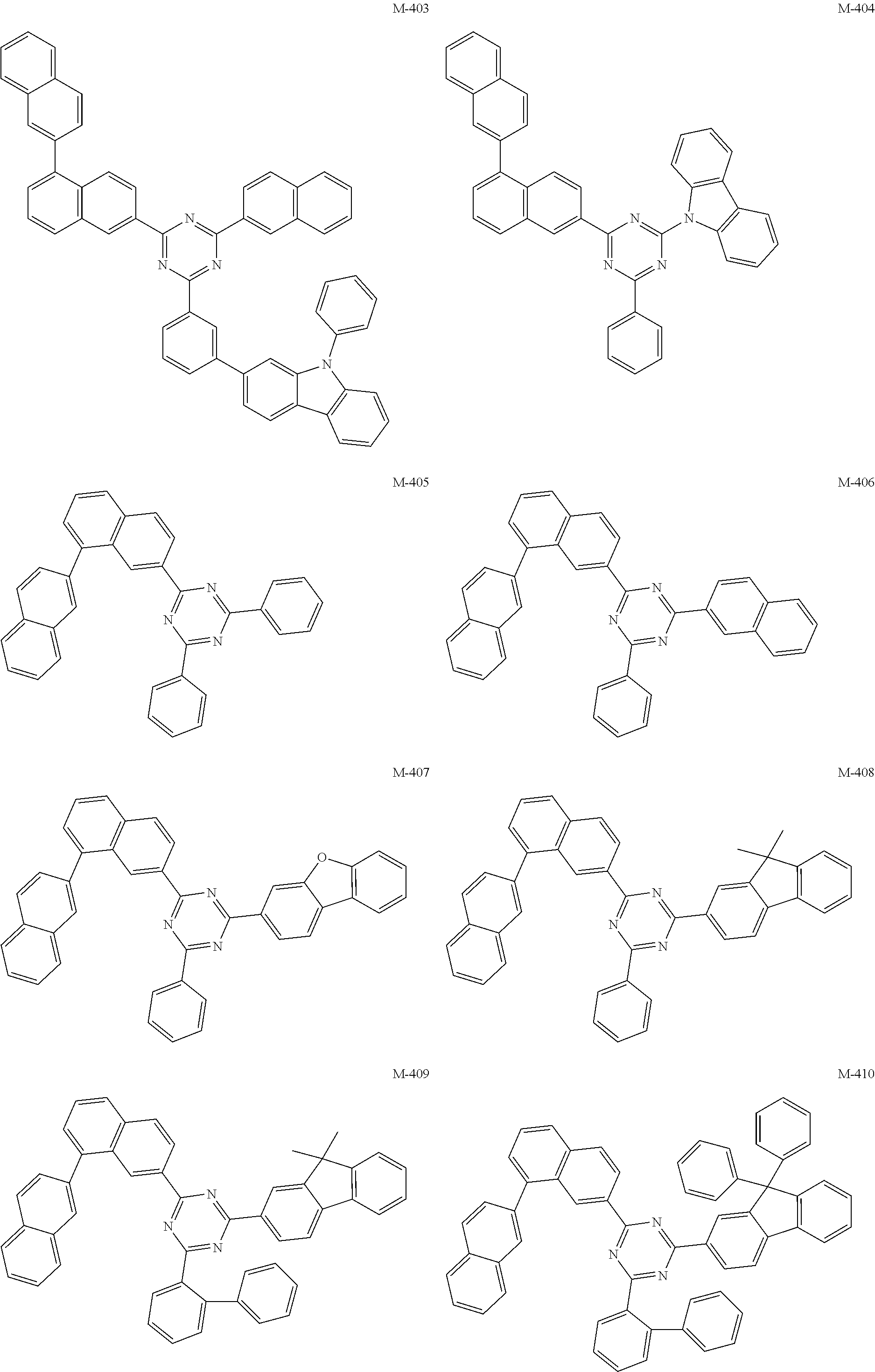

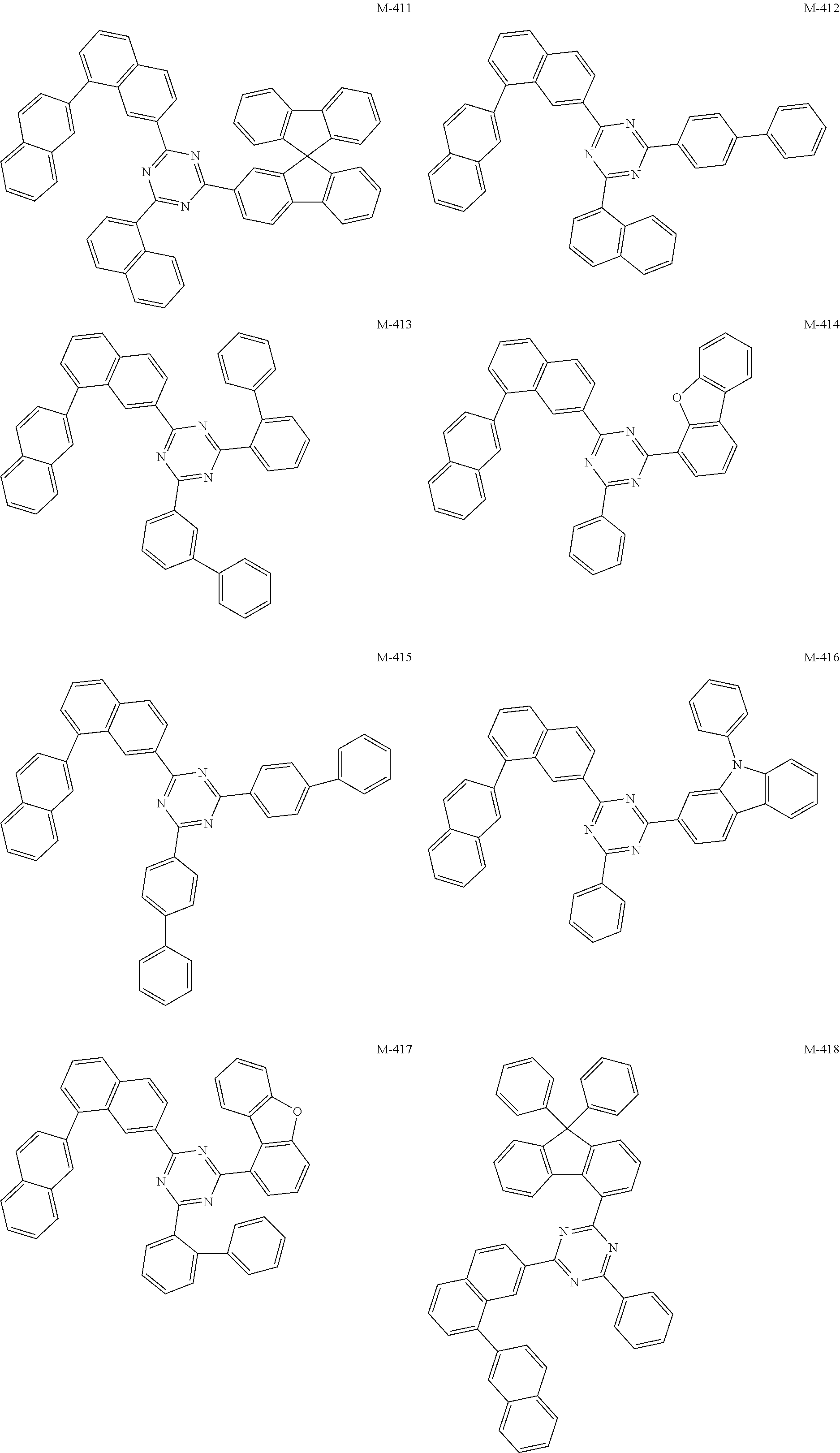

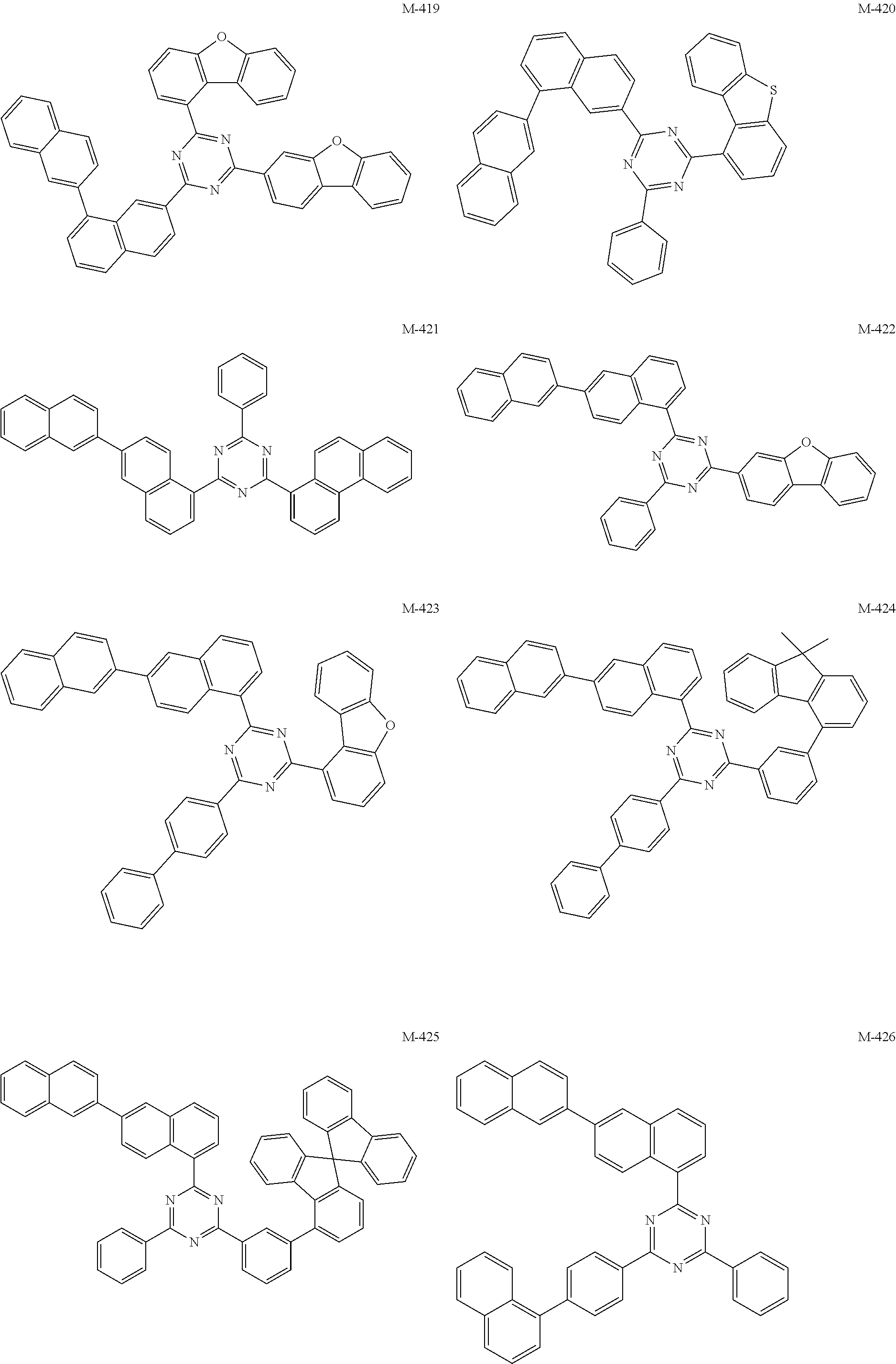

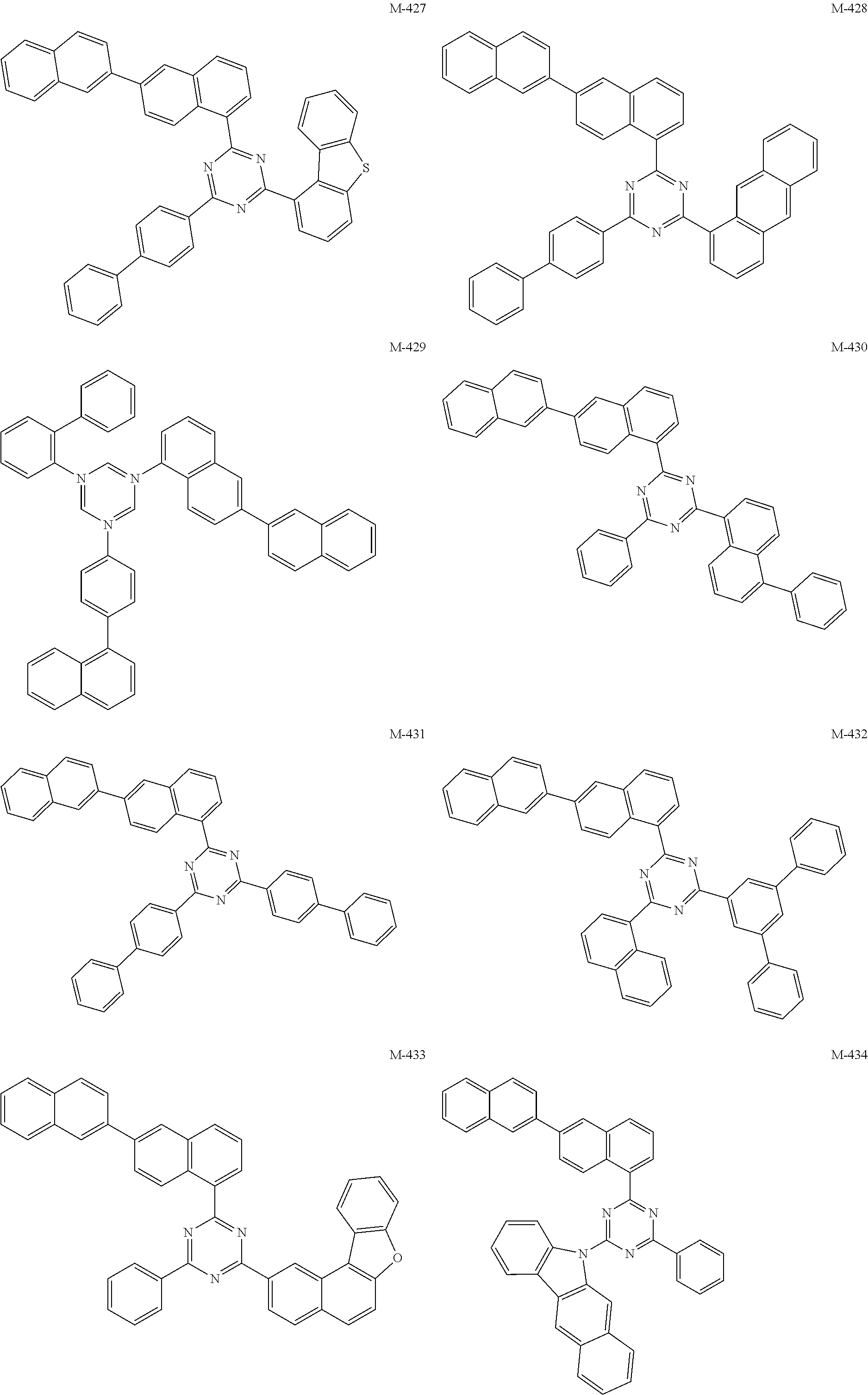

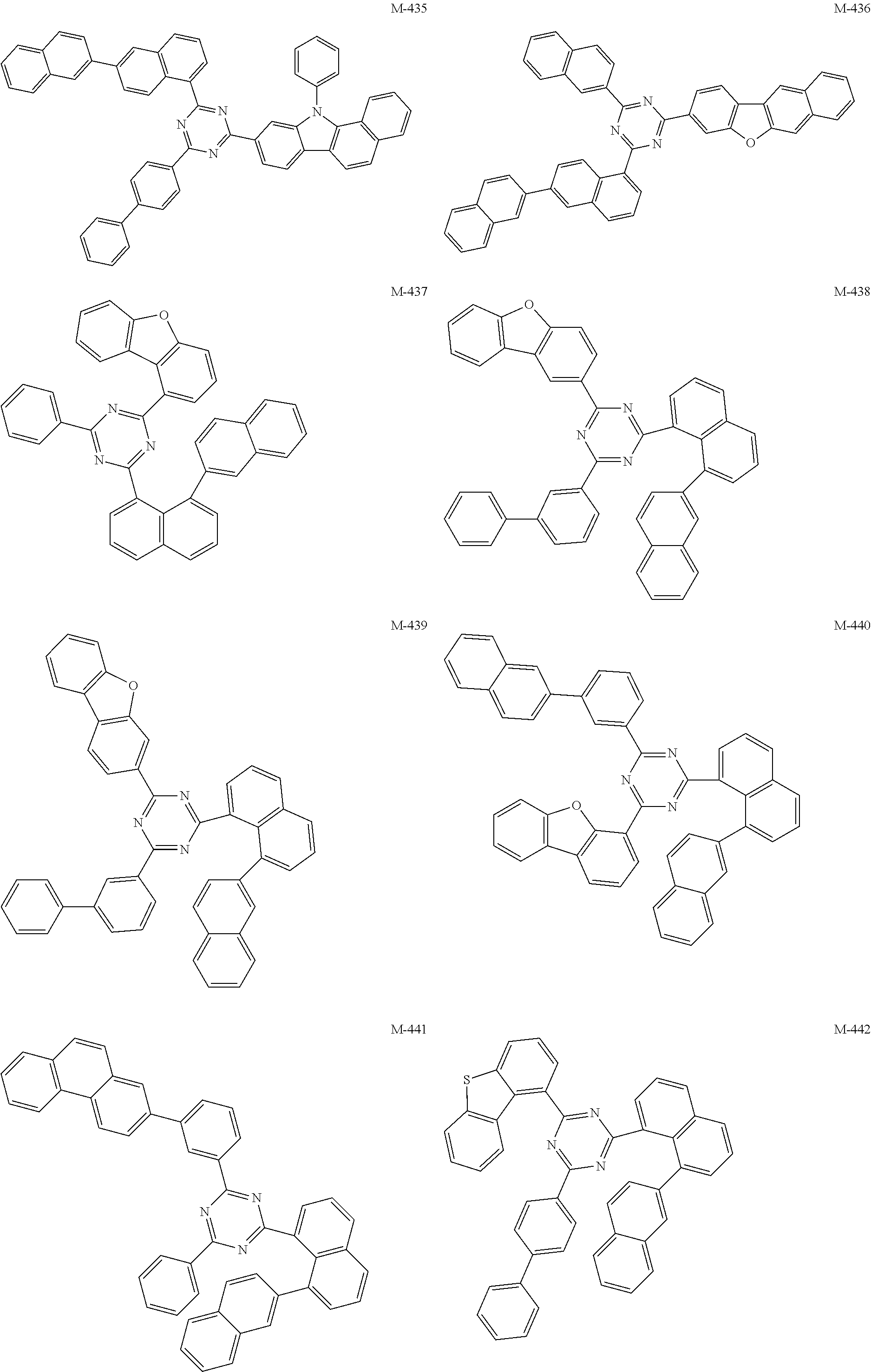

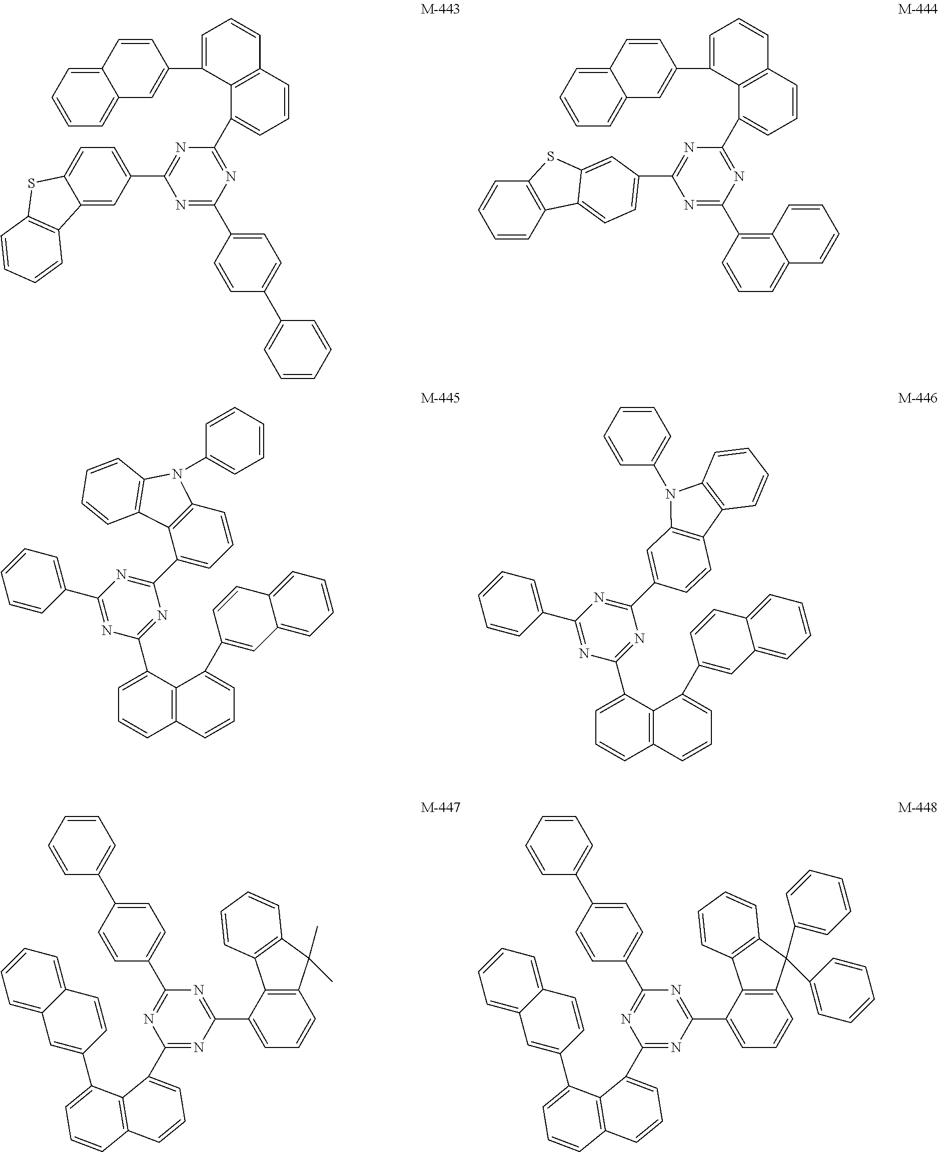

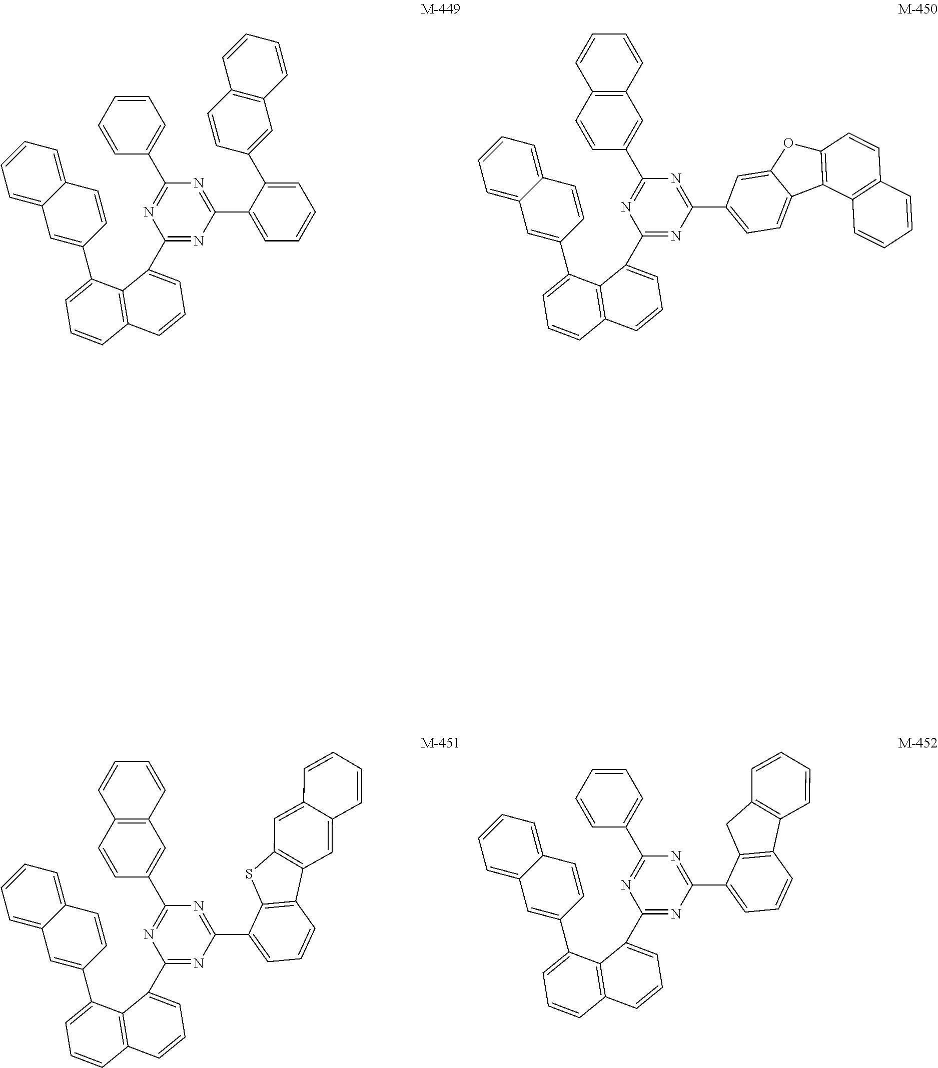 [SMILES: c1ccc(-c2nc(-c3ccccc3-c3ccc4ccccc4c3)nc(-c3cccc4cccc(-c5ccc6ccccc6c5)c34)n2)cc1 ; c1ccc2cc(-c3nc(-c4ccc5c(c4)oc4ccc6ccccc6c45)nc(-c4cccc5cccc(-c6ccc7ccccc7c6)c45)n3)ccc2c1 ; c1ccc2cc(-c3nc(-c4cccc5c4sc4cc6ccccc6cc45)nc(-c4cccc5cccc(-c6ccc7ccccc7c6)c45)n3)ccc2c1 ; c1ccc(-c2nc(-c3cccc4c3Cc3ccccc3-4)nc(-c3cccc4cccc(-c5ccc6ccccc6c5)c34)n2)cc1]

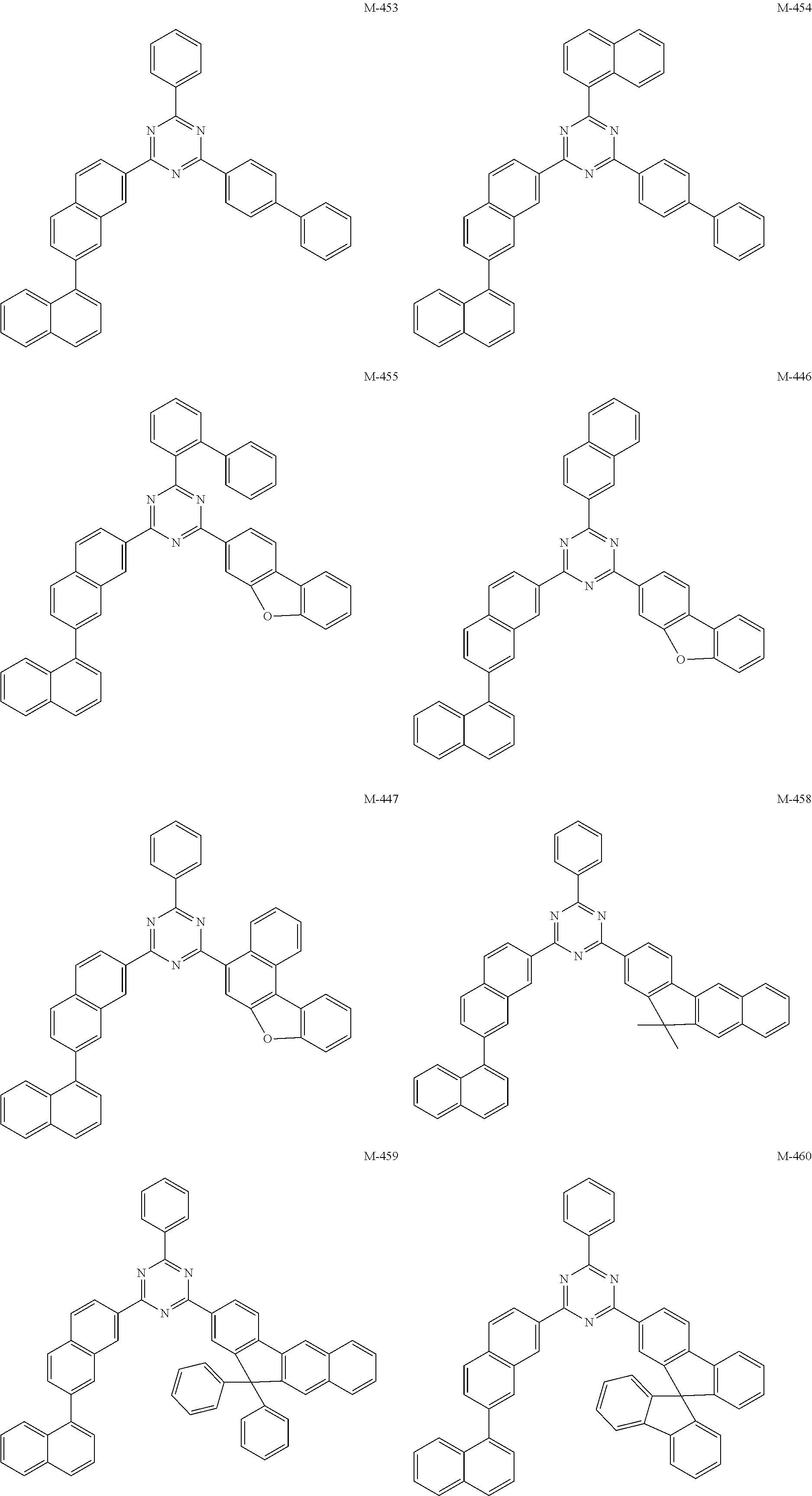

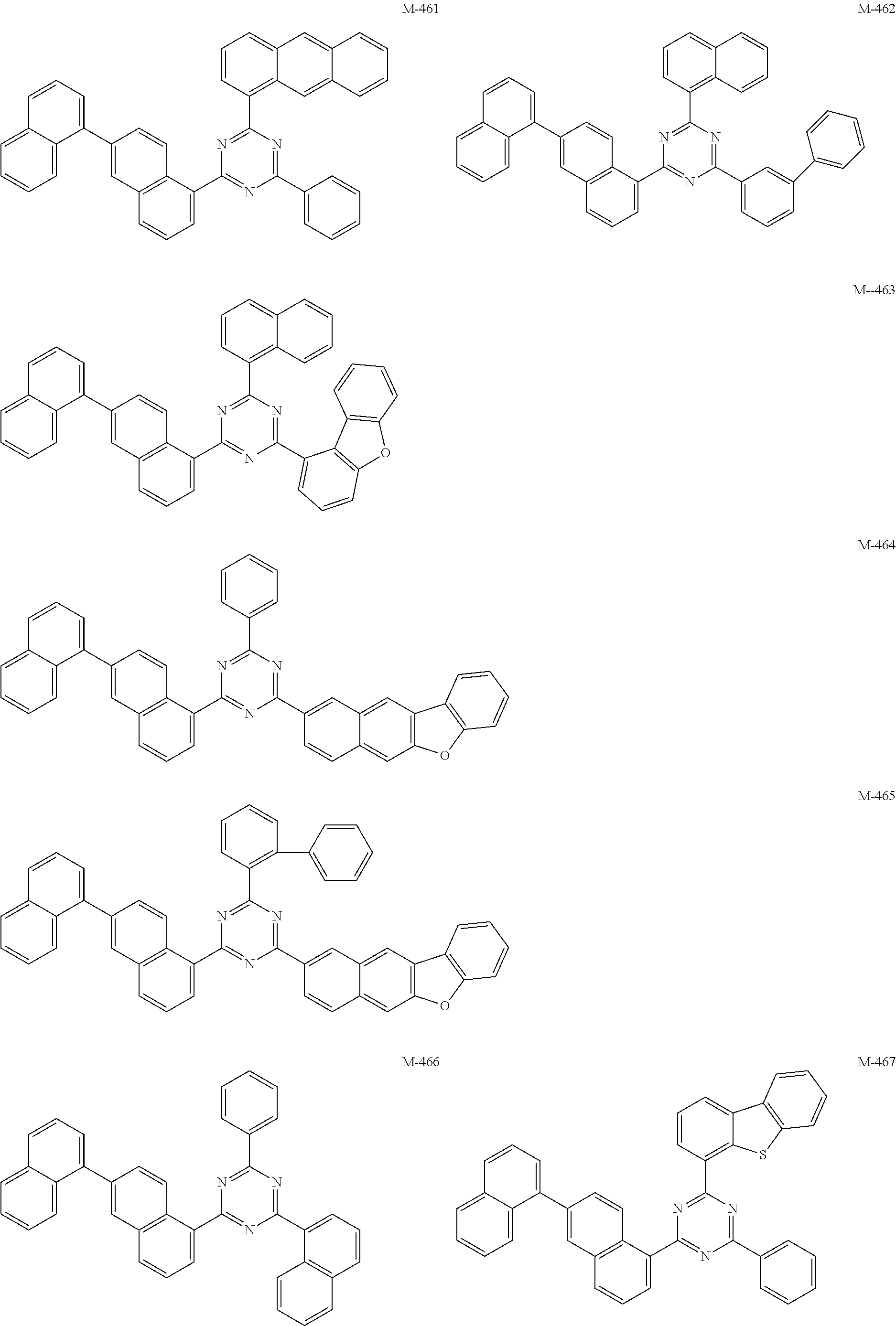

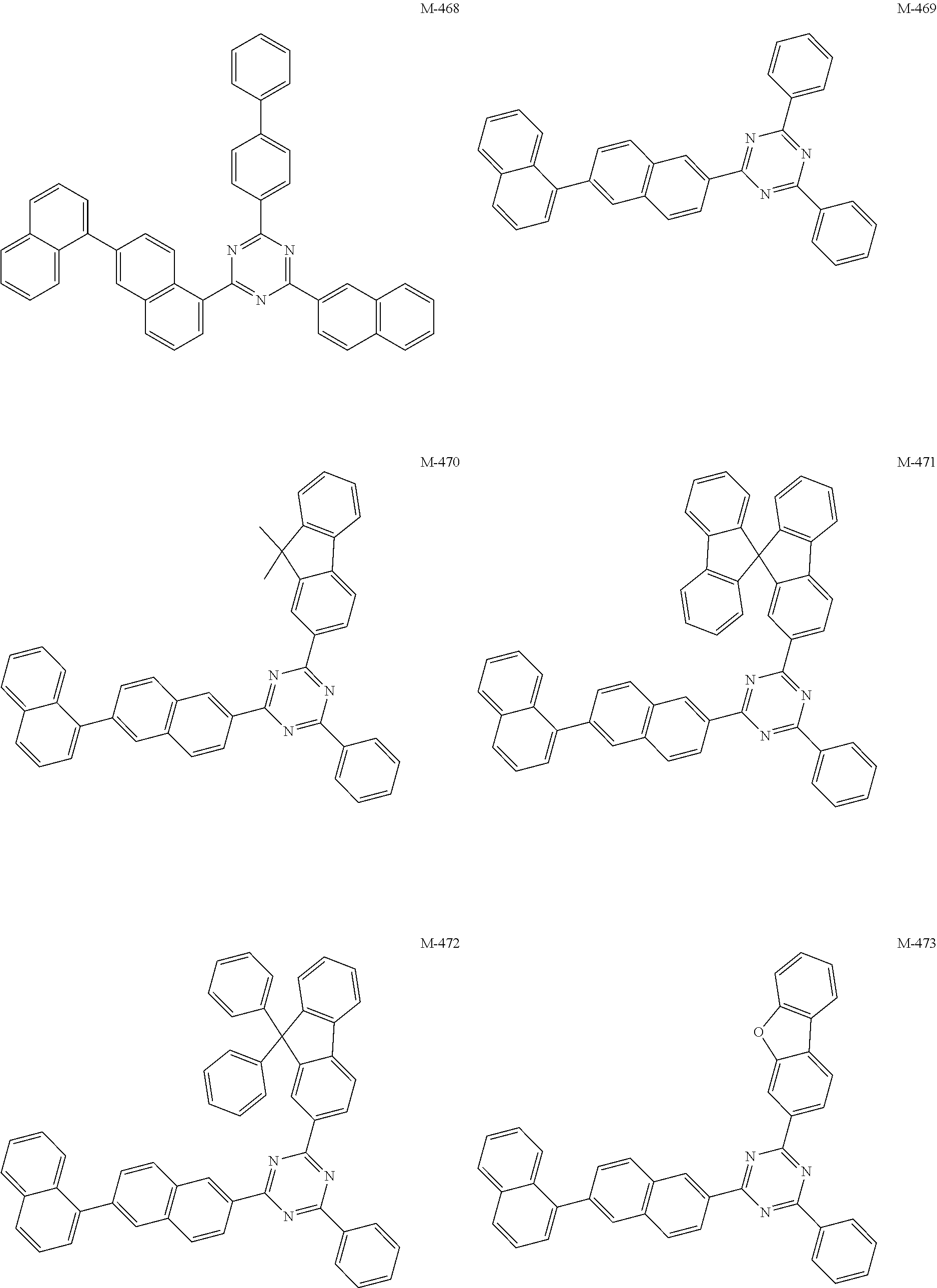

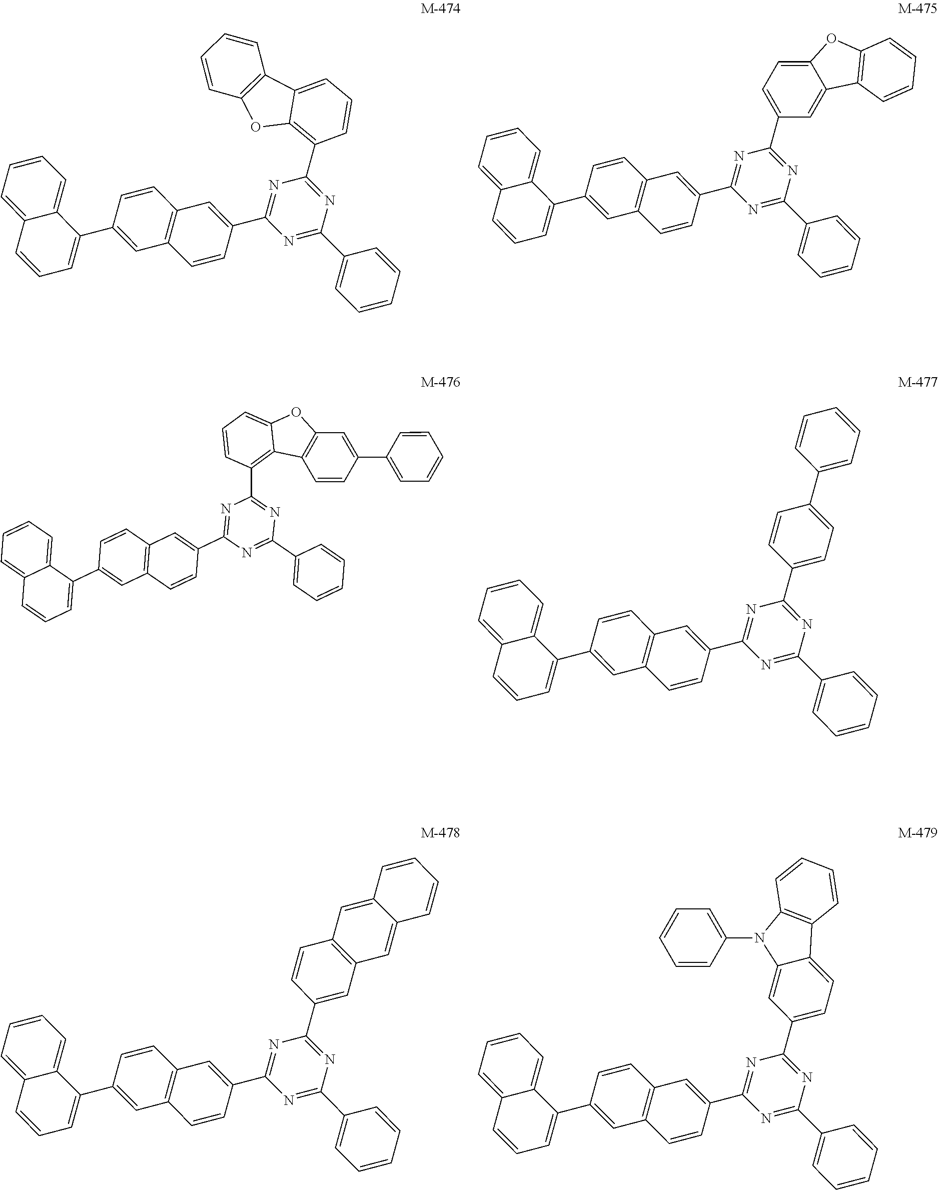 [SMILES: c1ccc(-c2nc(-c3ccc4cc(-c5cccc6ccccc56)ccc4c3)nc(-c3cccc4c3oc3ccccc34)n2)cc1 ; c1ccc(-c2nc(-c3ccc4cc(-c5cccc6ccccc56)ccc4c3)nc(-c3ccc4oc5ccccc5c4c3)n2)cc1 ; c1ccc(-c2ccc3c(c2)oc2cccc(-c4nc(-c5ccccc5)nc(-c5ccc6cc(-c7cccc8ccccc78)ccc6c5)n4)c23)cc1 ; c1ccc(-c2ccc(-c3nc(-c4ccccc4)nc(-c4ccc5cc(-c6cccc7ccccc67)ccc5c4)n3)cc2)cc1 ; c1ccc(-c2nc(-c3ccc4cc(-c5cccc6ccccc56)ccc4c3)nc(-c3ccc4cc5ccccc5cc4c3)n2)cc1 ; c1ccc(-c2nc(-c3ccc4cc(-c5cccc6ccccc56)ccc4c3)nc(-c3ccc4c5ccccc5n(-c5ccccc5)c4c3)n2)cc1]

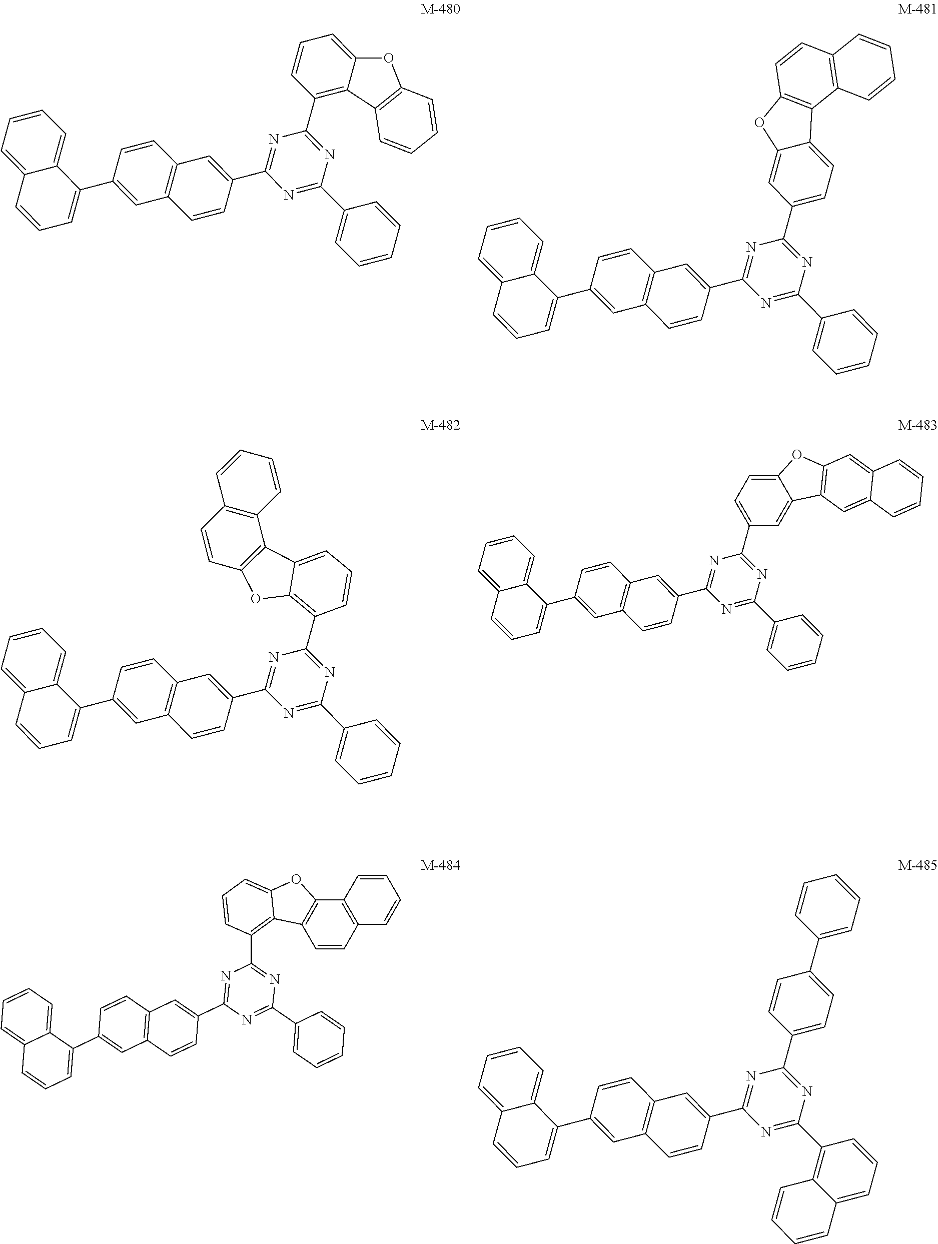 [SMILES: c1ccc(-c2nc(-c3ccc4cc(-c5cccc6ccccc56)ccc4c3)nc(-c3cccc4oc5ccccc5c34)n2)cc1 ; c1ccc(-c2nc(-c3ccc4cc(-c5cccc6ccccc56)ccc4c3)nc(-c3ccc4c(c3)oc3ccc5ccccc5c34)n2)cc1 ; c1ccc(-c2nc(-c3ccc4cc(-c5cccc6ccccc56)ccc4c3)nc(-c3cccc4c3oc3ccc5ccccc5c34)n2)cc1 ; c1ccc(-c2nc(-c3ccc4cc(-c5cccc6ccccc56)ccc4c3)nc(-c3ccc4oc5cc6ccccc6cc5c4c3)n2)cc1 ; c1ccc(-c2nc(-c3ccc4cc(-c5cccc6ccccc56)ccc4c3)nc(-c3cccc4oc5c6ccccc6ccc5c34)n2)cc1 ; c1ccc(-c2ccc(-c3nc(-c4ccc5cc(-c6cccc7ccccc67)ccc5c4)nc(-c4cccc5ccccc45)n3)cc2)cc1]

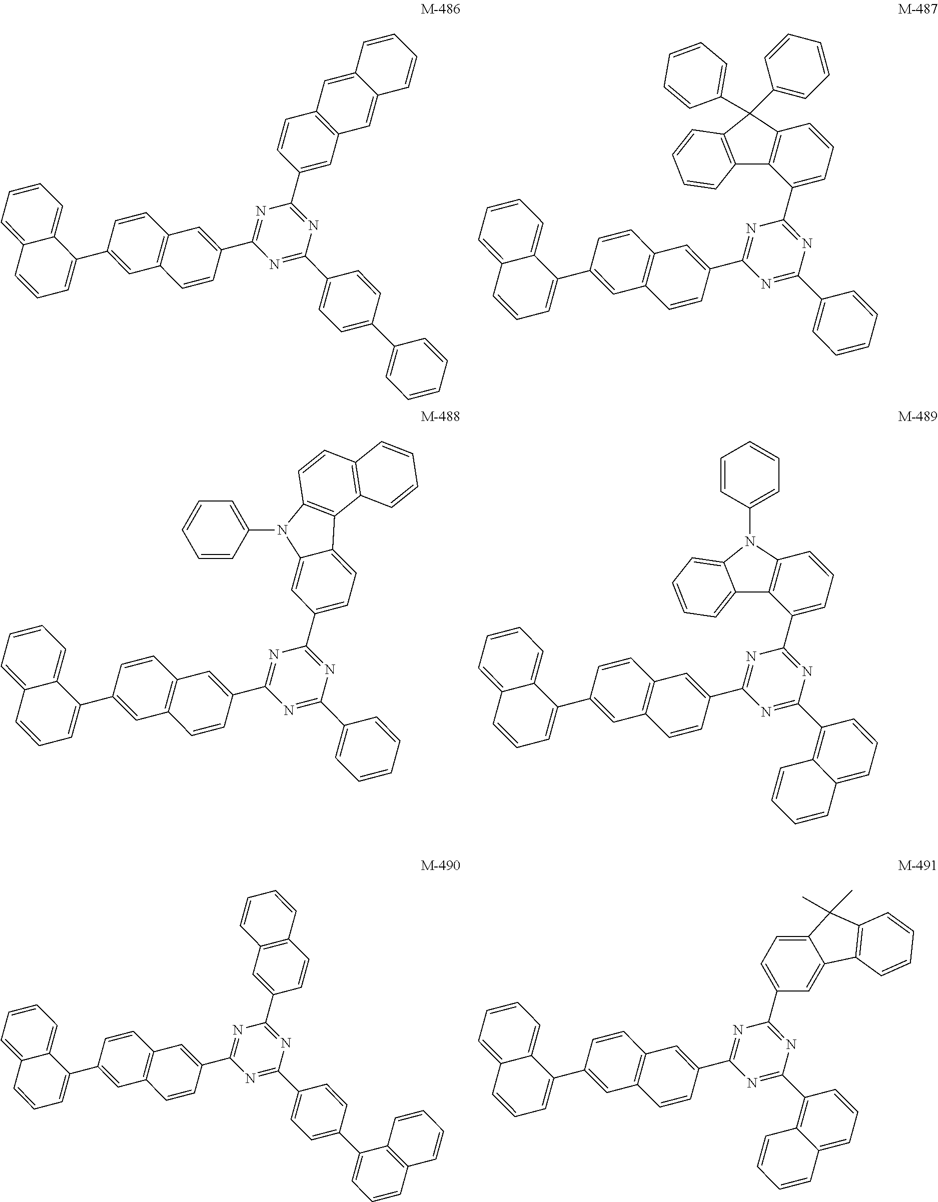

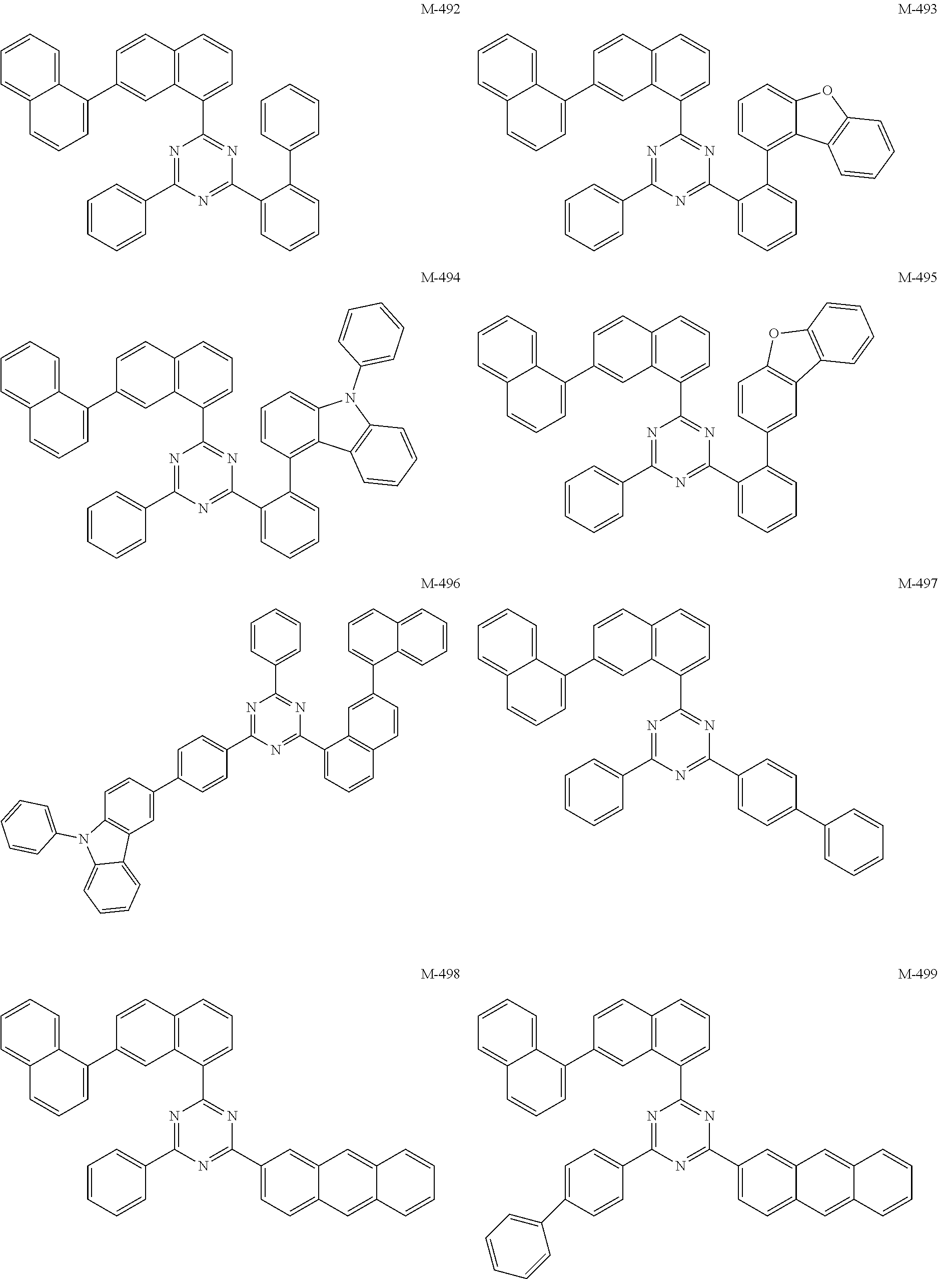

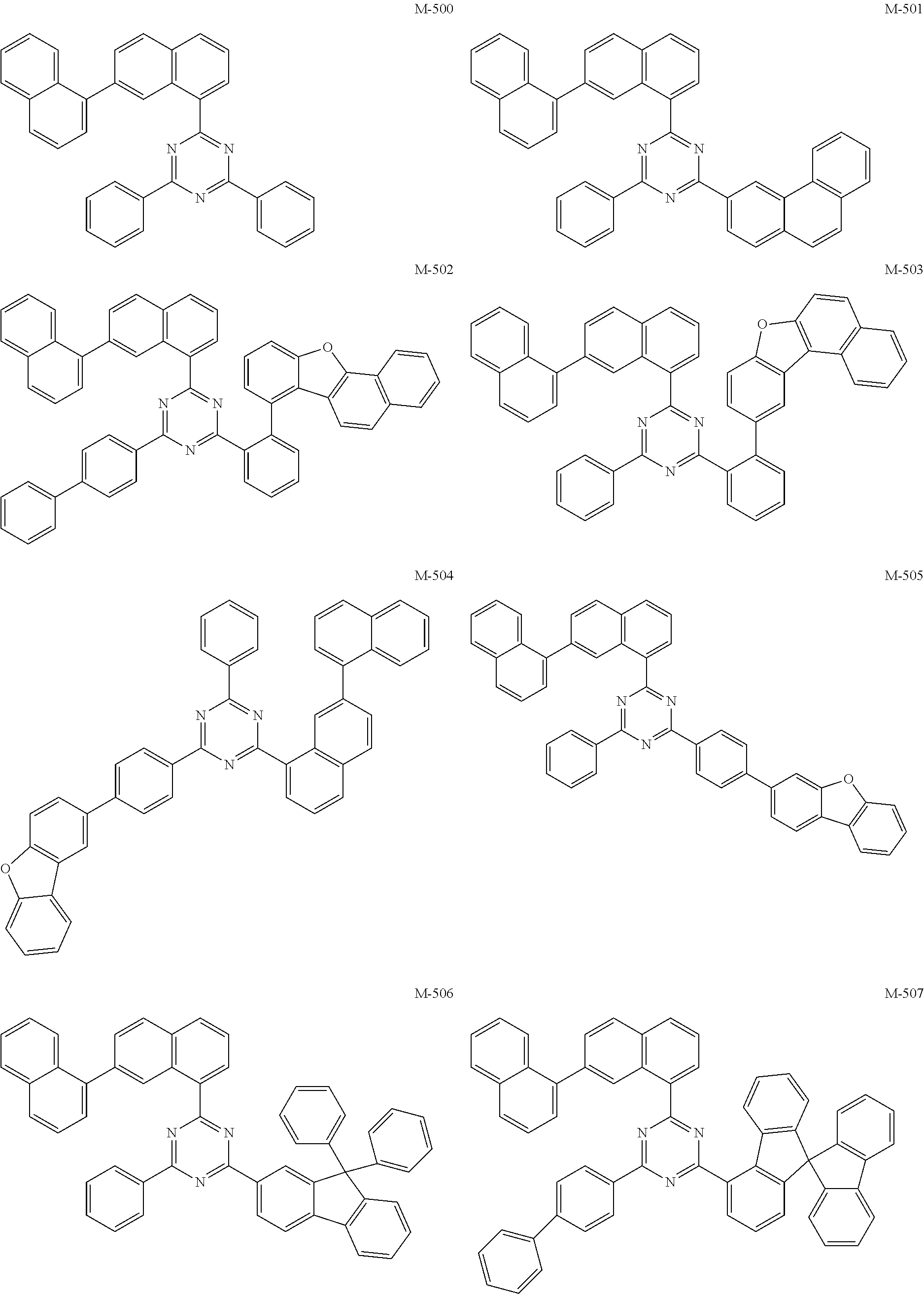

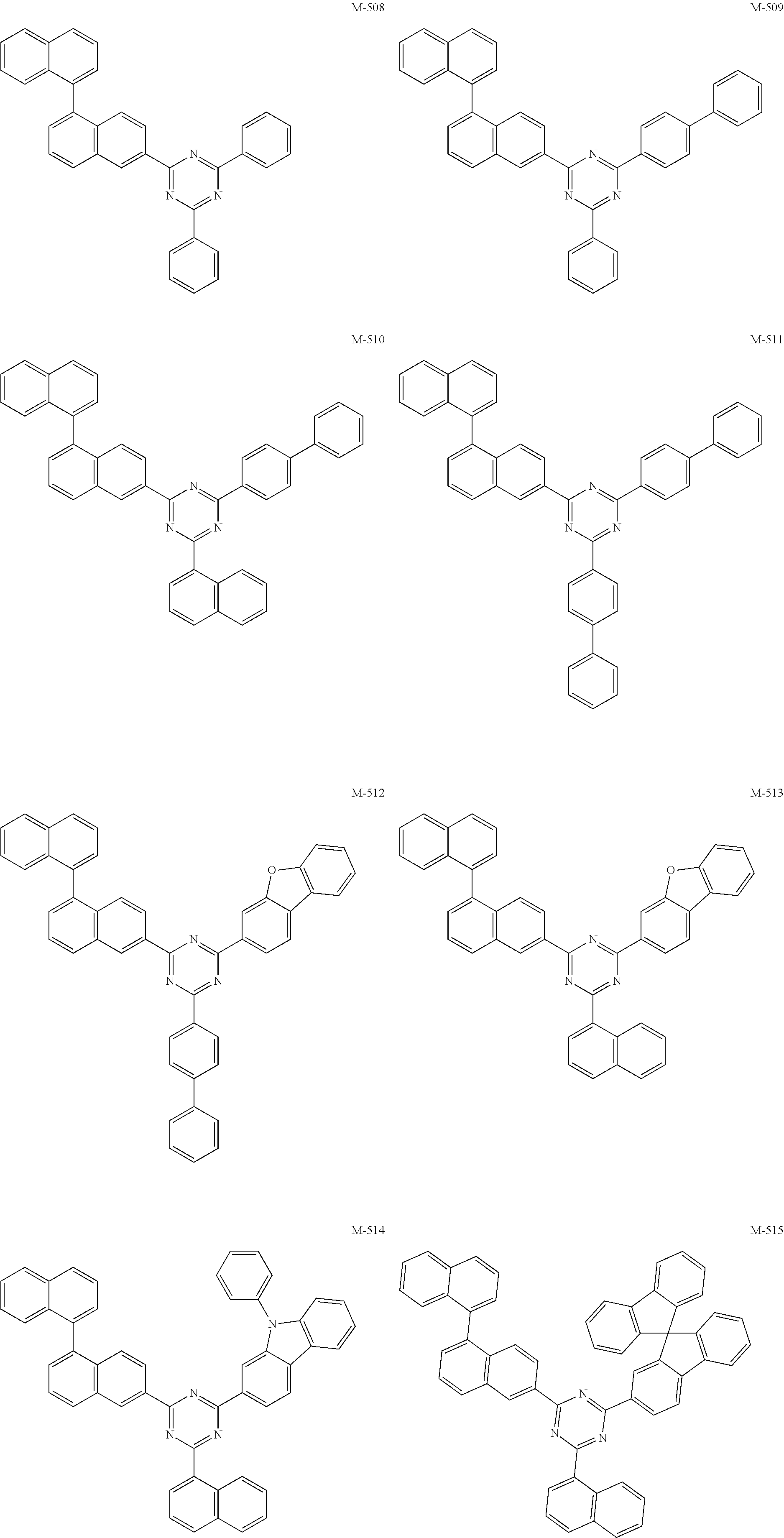

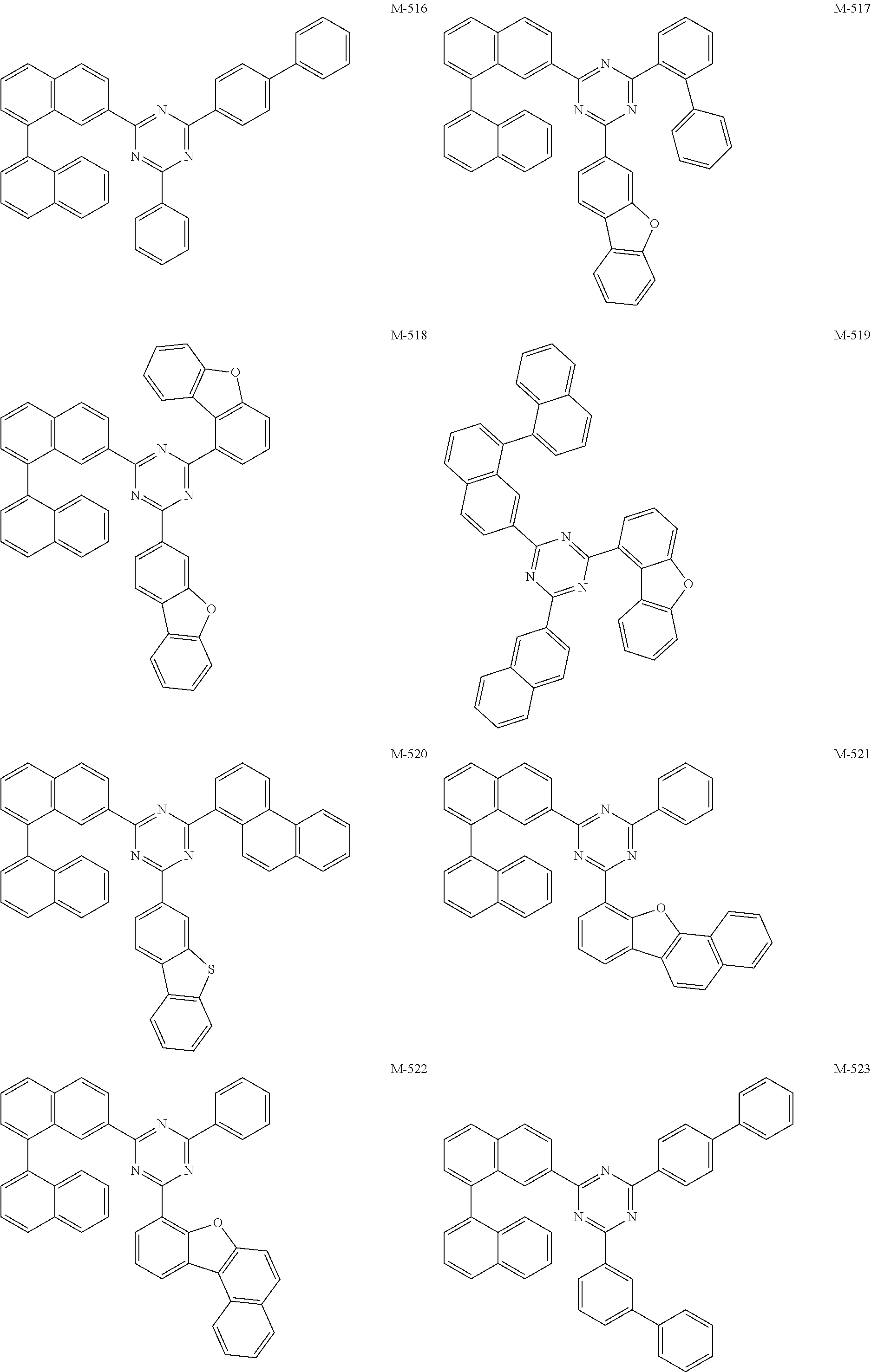

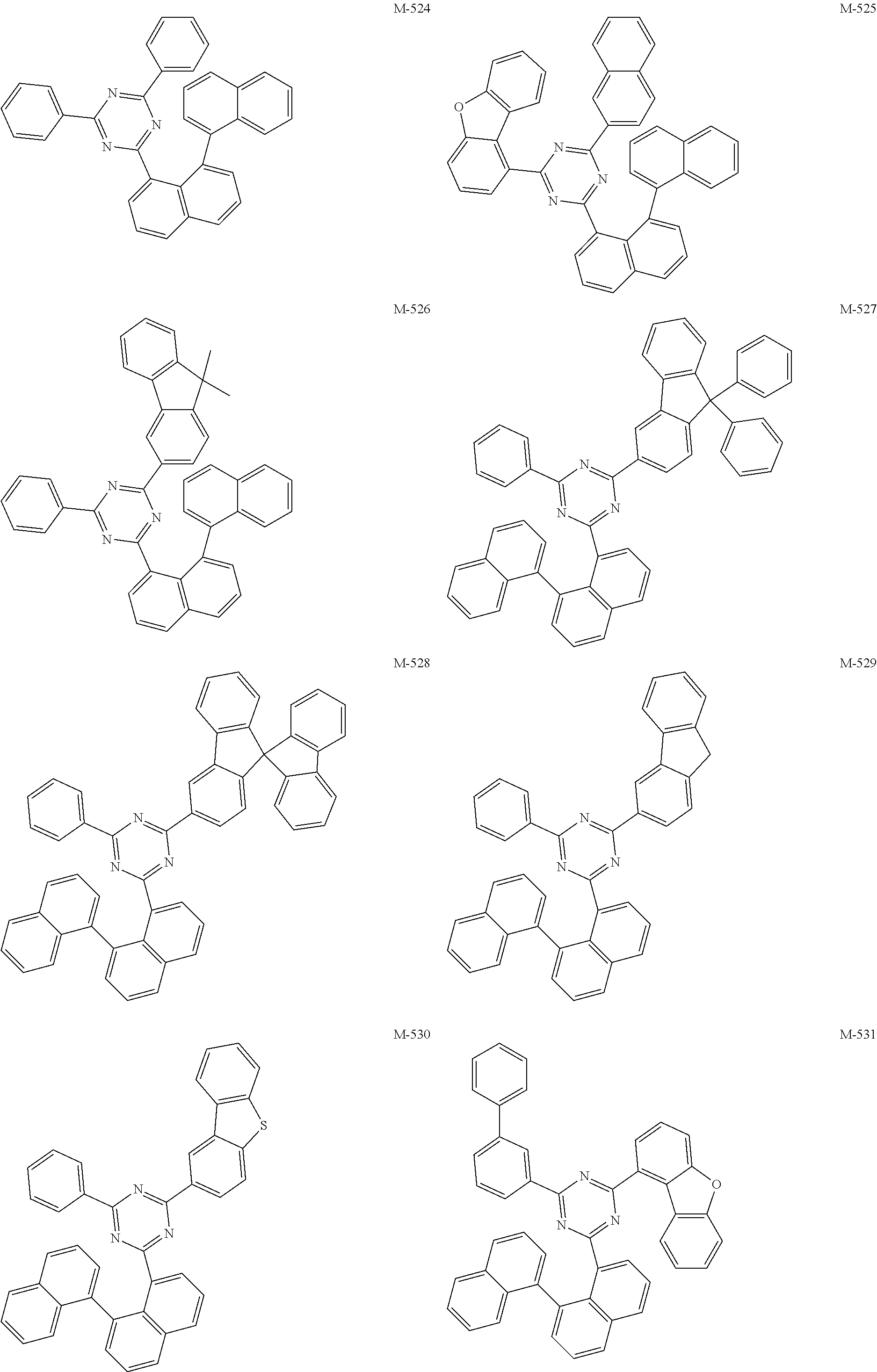

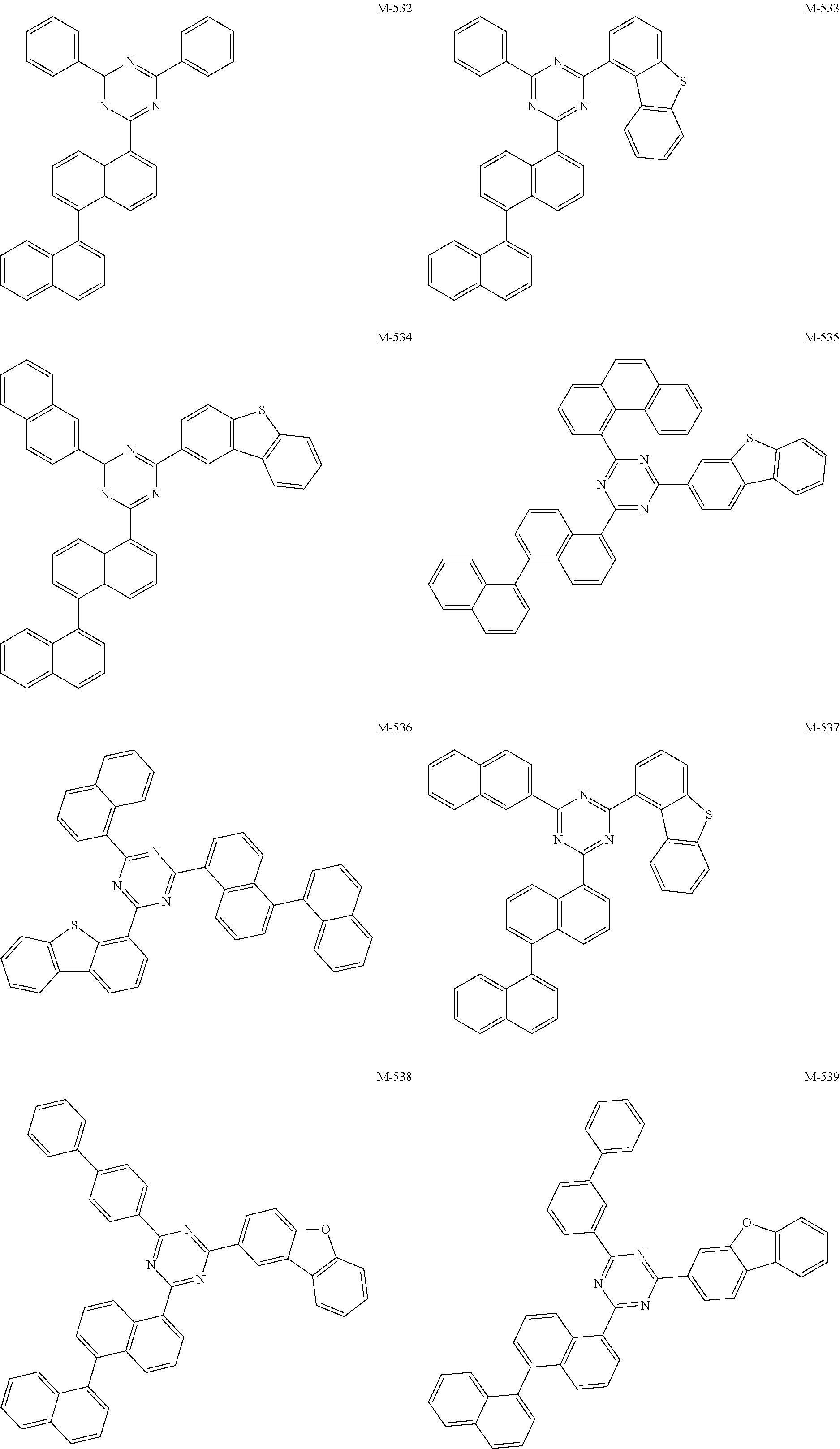

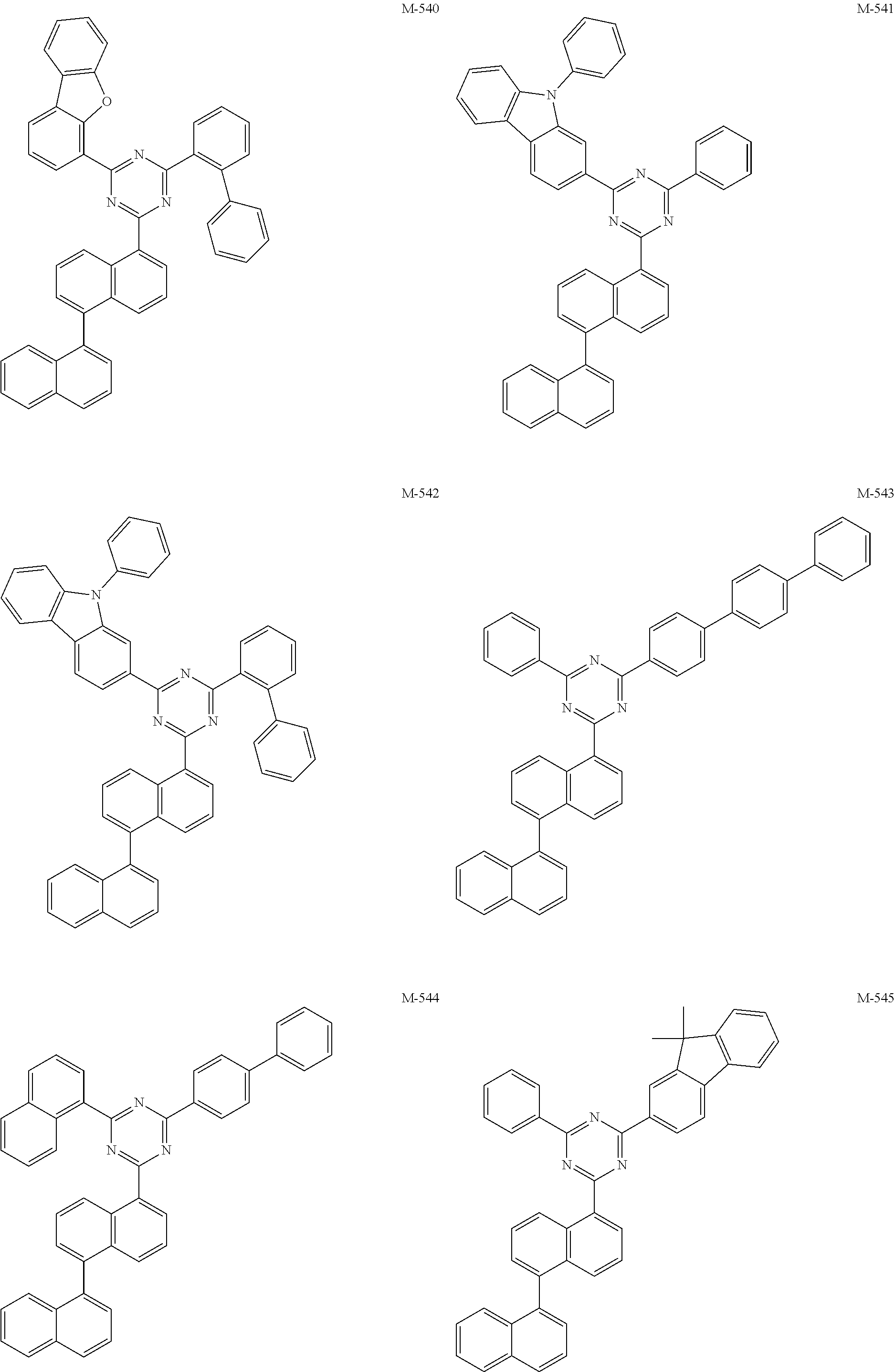

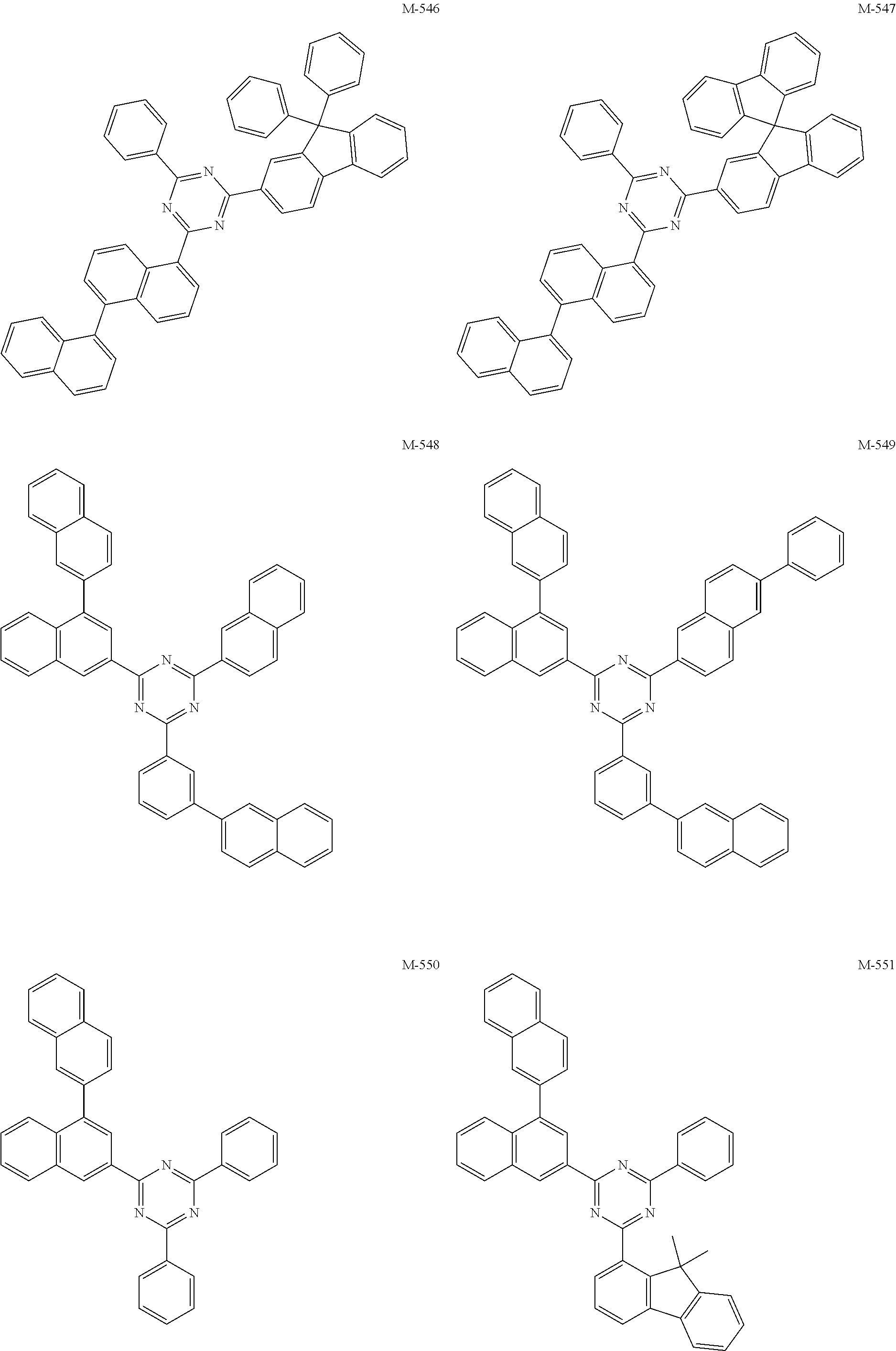

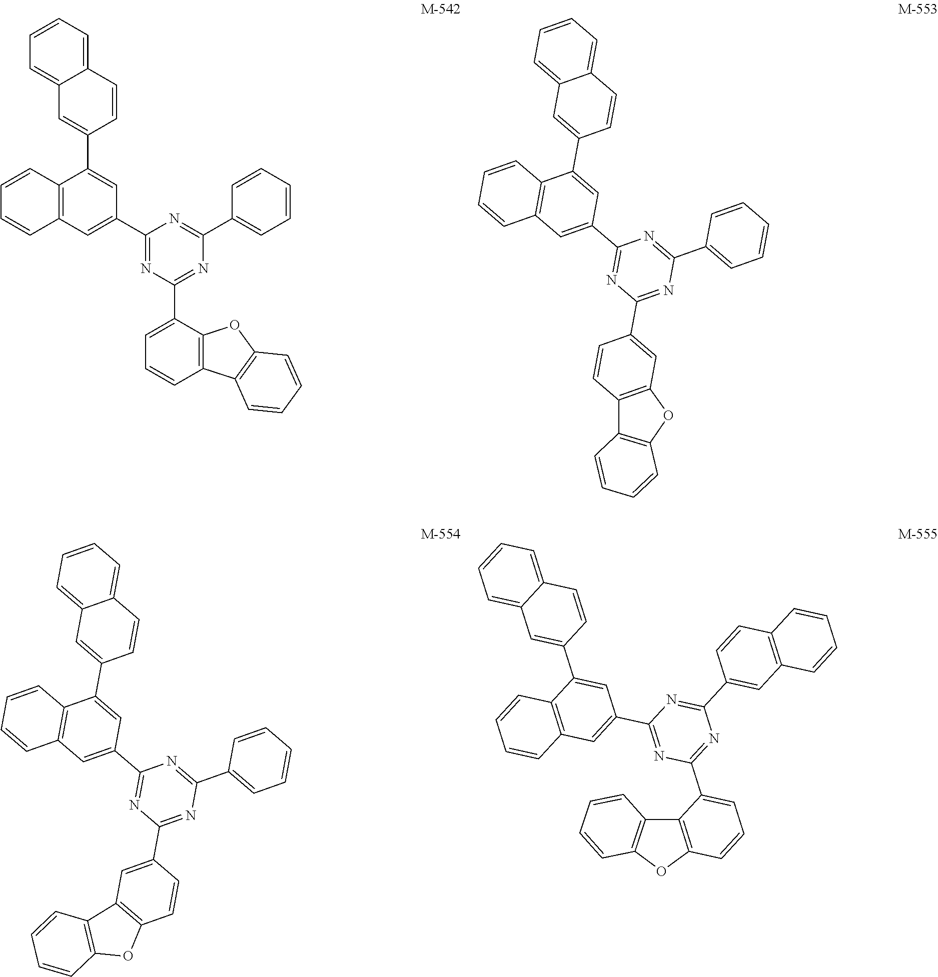 [SMILES: c1ccc(-c2nc(-c3cc(-c4ccc5ccccc5c4)c4ccccc4c3)nc(-c3cccc4c3oc3ccccc34)n2)cc1 ; c1ccc(-c2nc(-c3cc(-c4ccc5ccccc5c4)c4ccccc4c3)nc(-c3ccc4c(c3)oc3ccccc34)n2)cc1 ; c1ccc(-c2nc(-c3cc(-c4ccc5ccccc5c4)c4ccccc4c3)nc(-c3ccc4oc5ccccc5c4c3)n2)cc1 ; c1ccc2cc(-c3nc(-c4cc(-c5ccc6ccccc6c5)c5ccccc5c4)nc(-c4cccc5oc6ccccc6c45)n3)ccc2c1]

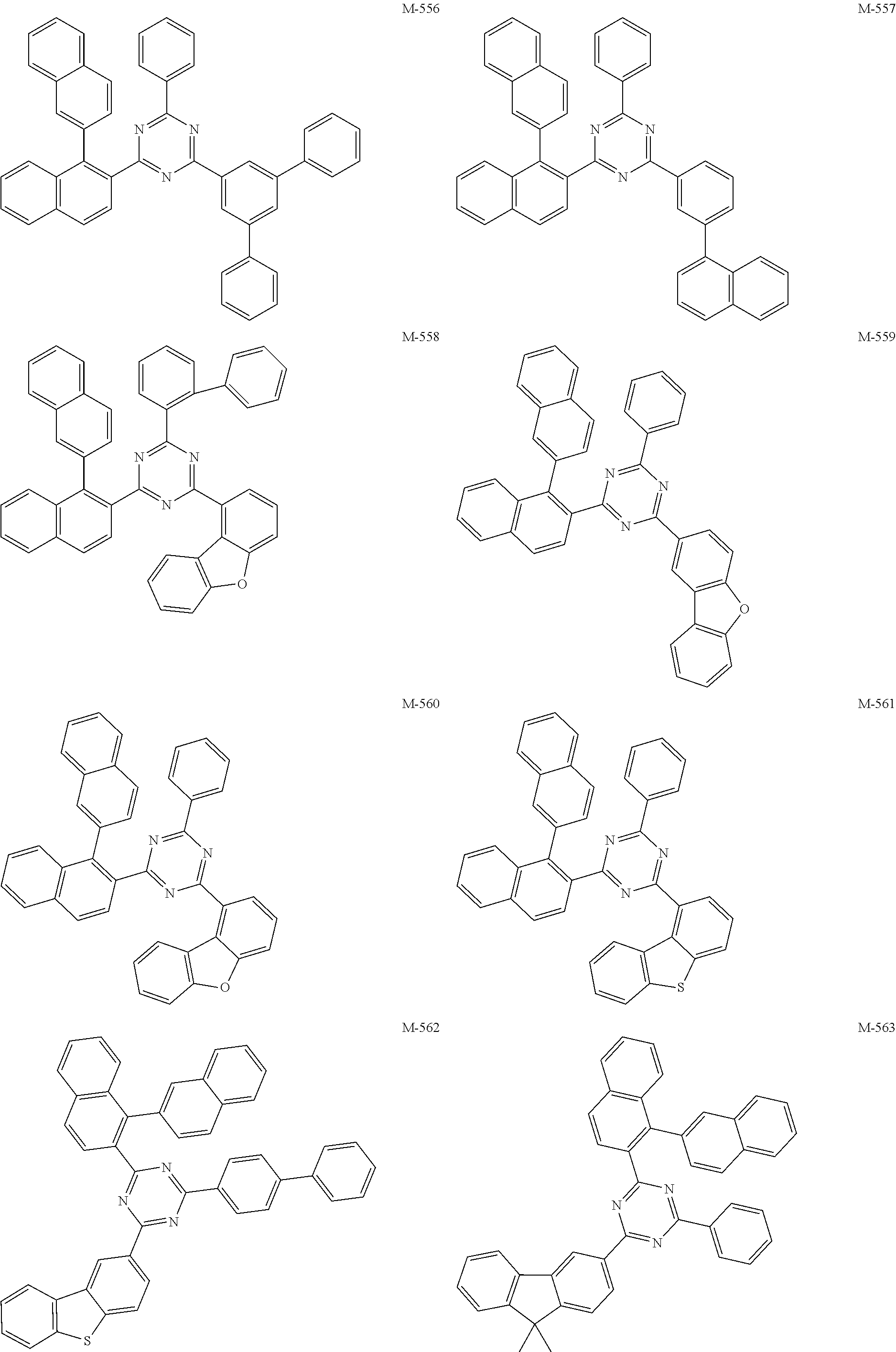

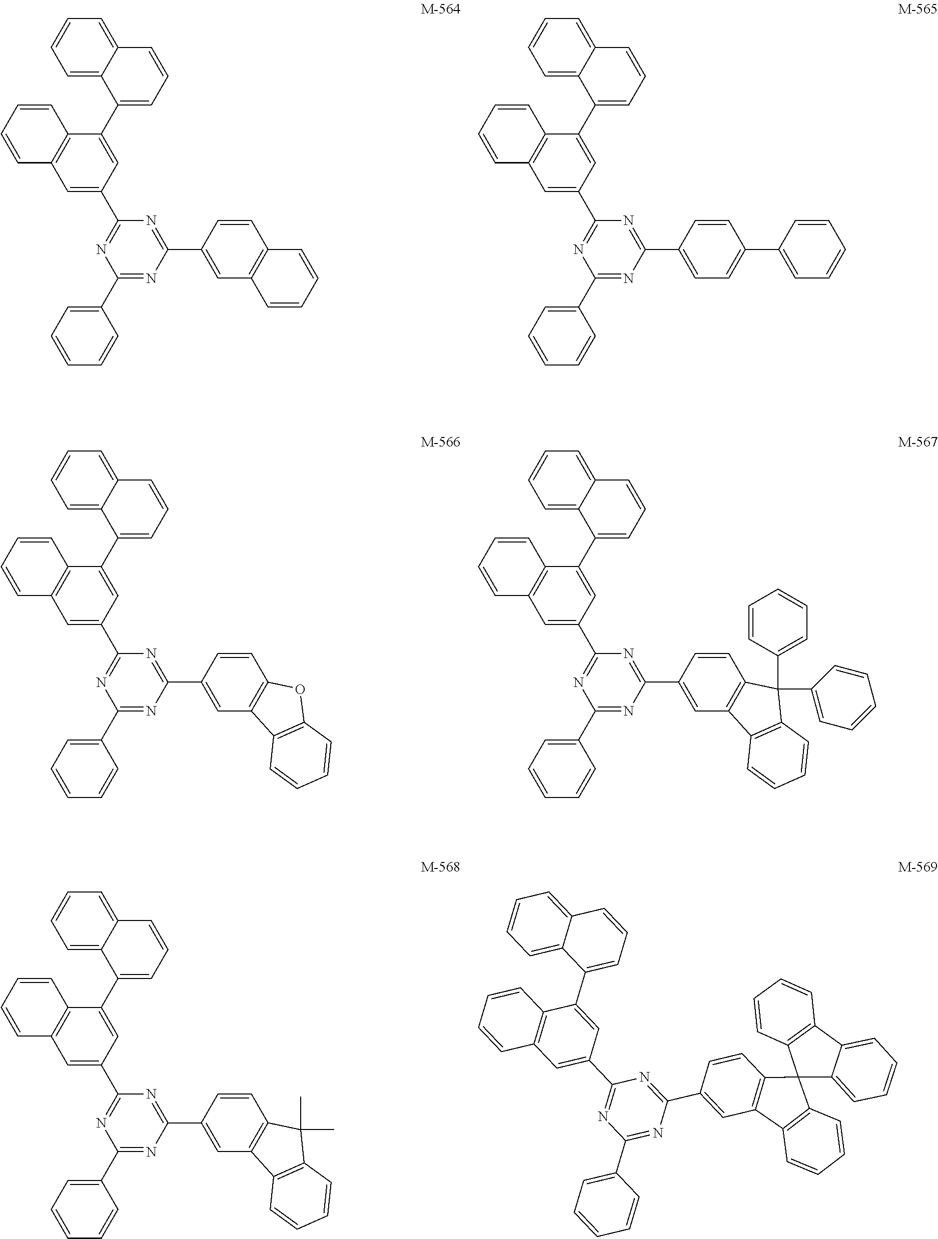

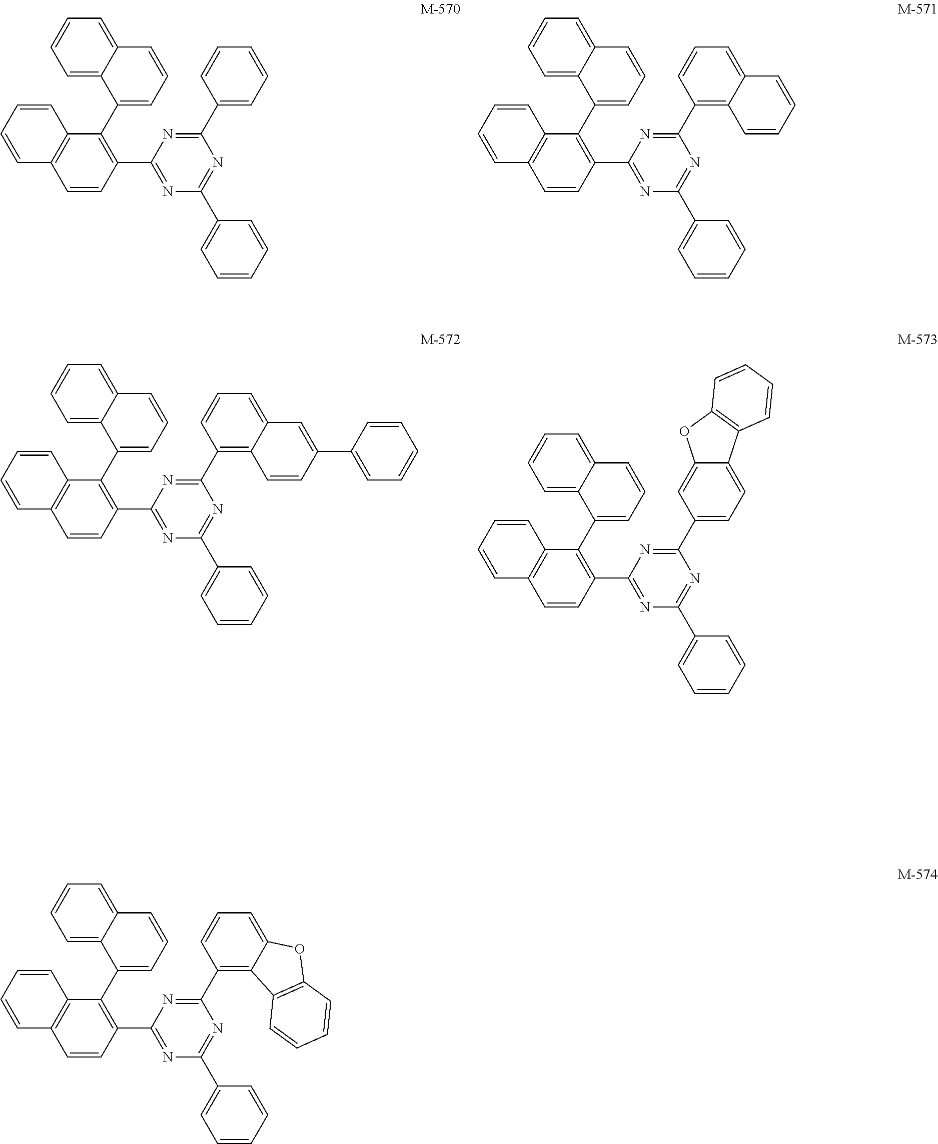 [SMILES: c1ccc(-c2nc(-c3ccccc3)nc(-c3ccc4ccccc4c3-c3cccc4ccccc34)n2)cc1 ; c1ccc(-c2nc(-c3ccc4ccccc4c3-c3cccc4ccccc34)nc(-c3cccc4ccccc34)n2)cc1 ; c1ccc(-c2ccc3c(-c4nc(-c5ccccc5)nc(-c5ccc6ccccc6c5-c5cccc6ccccc56)n4)cccc3c2)cc1 ; c1ccc(-c2nc(-c3ccc4c(c3)oc3ccccc34)nc(-c3ccc4ccccc4c3-c3cccc4ccccc34)n2)cc1 ; c1ccc(-c2nc(-c3ccc4ccccc4c3-c3cccc4ccccc34)nc(-c3cccc4oc5ccccc5c34)n2)cc1]

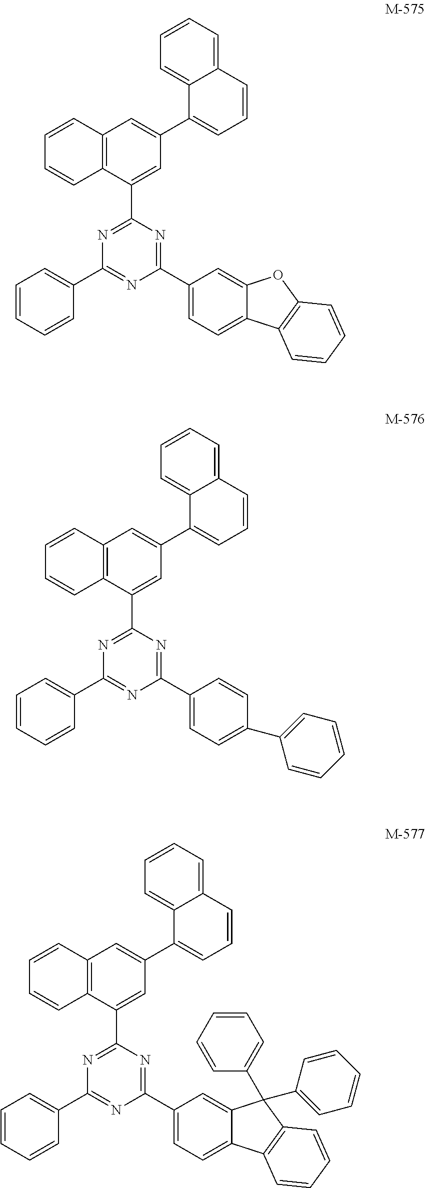 [SMILES: c1ccc(-c2nc(-c3ccc4c(c3)oc3ccccc34)nc(-c3cc(-c4cccc5ccccc45)cc4ccccc34)n2)cc1 ; c1ccc(-c2ccc(-c3nc(-c4ccccc4)nc(-c4cc(-c5cccc6ccccc56)cc5ccccc45)n3)cc2)cc1 ; c1ccc(-c2nc(-c3ccc4c(c3)C(c3ccccc3)(c3ccccc3)c3ccccc3-4)nc(-c3cc(-c4cccc5ccccc45)cc4ccccc34)n2)cc1]

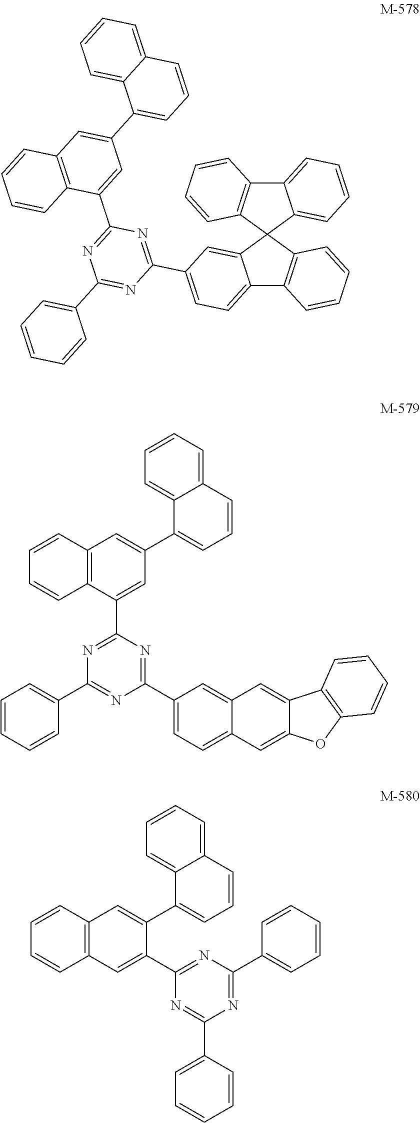 [SMILES: c1ccc(-c2nc(-c3ccc4c(c3)C3(c5ccccc5-c5ccccc53)c3ccccc3-4)nc(-c3cc(-c4cccc5ccccc45)cc4ccccc34)n2)cc1 ; c1ccc(-c2nc(-c3ccc4cc5oc6ccccc6c5cc4c3)nc(-c3cc(-c4cccc5ccccc45)cc4ccccc34)n2)cc1 ; c1ccc(-c2nc(-c3ccccc3)nc(-c3cc4ccccc4cc3-c3cccc4ccccc34)n2)cc1]

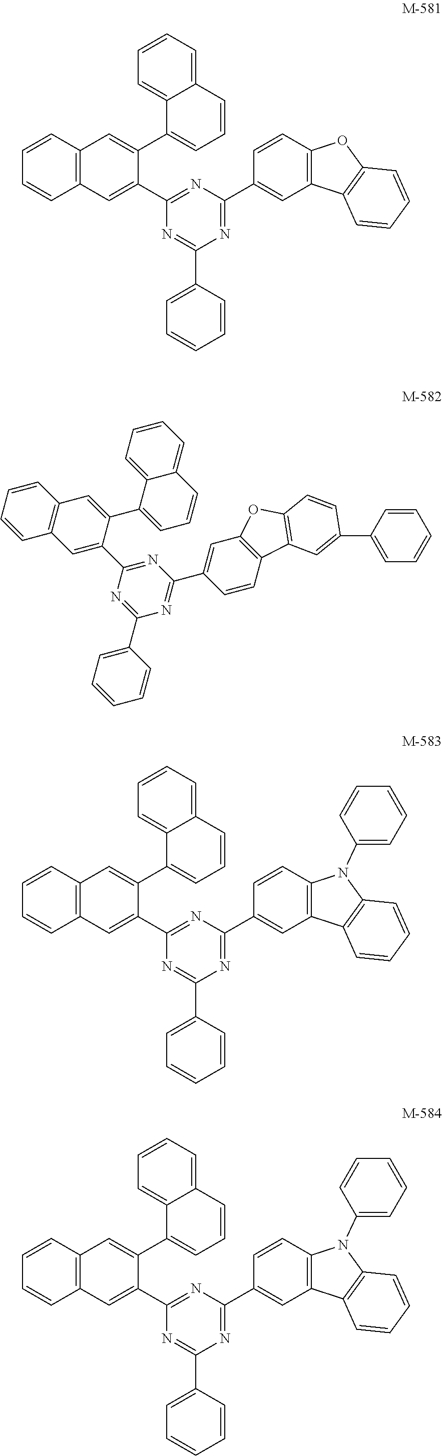 [SMILES: c1ccc(-c2nc(-c3ccc4oc5ccccc5c4c3)nc(-c3cc4ccccc4cc3-c3cccc4ccccc34)n2)cc1 ; c1ccc(-c2ccc3oc4cc(-c5nc(-c6ccccc6)nc(-c6cc7ccccc7cc6-c6cccc7ccccc67)n5)ccc4c3c2)cc1 ; c1ccc(-c2nc(-c3ccc4c(c3)c3ccccc3n4-c3ccccc3)nc(-c3cc4ccccc4cc3-c3cccc4ccccc34)n2)cc1 ; c1ccc(-c2nc(-c3ccc4c(c3)c3ccccc3n4-c3ccccc3)nc(-c3cc4ccccc4cc3-c3cccc4ccccc34)n2)cc1]

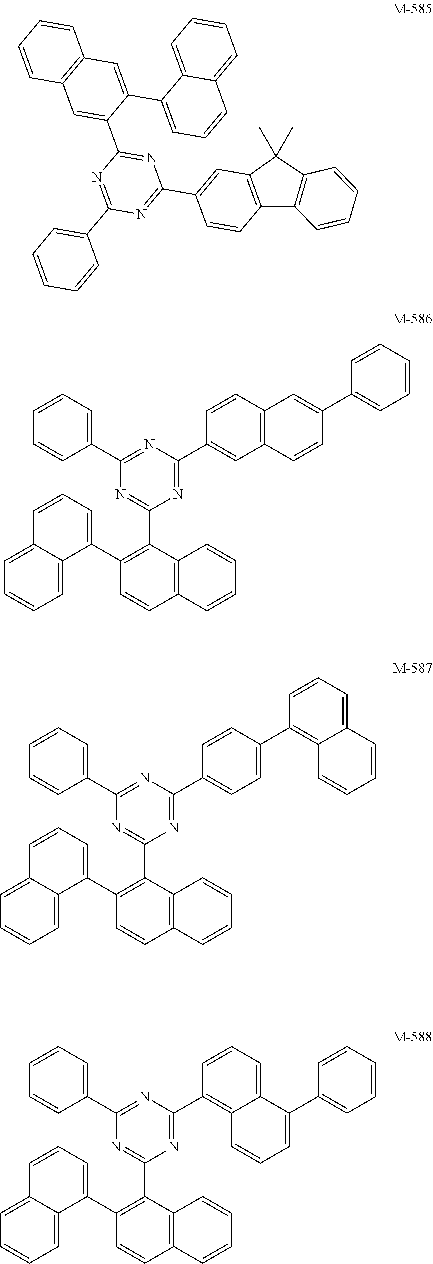 [SMILES: CC1(C)c2ccccc2-c2ccc(-c3nc(-c4ccccc4)nc(-c4cc5ccccc5cc4-c4cccc5ccccc45)n3)cc21 ; c1ccc(-c2ccc3cc(-c4nc(-c5ccccc5)nc(-c5c(-c6cccc7ccccc67)ccc6ccccc56)n4)ccc3c2)cc1 ; c1ccc(-c2nc(-c3ccc(-c4cccc5ccccc45)cc3)nc(-c3c(-c4cccc5ccccc45)ccc4ccccc34)n2)cc1 ; c1ccc(-c2nc(-c3c(-c4cccc5ccccc45)ccc4ccccc34)nc(-c3cccc4c(-c5ccccc5)cccc34)n2)cc1]

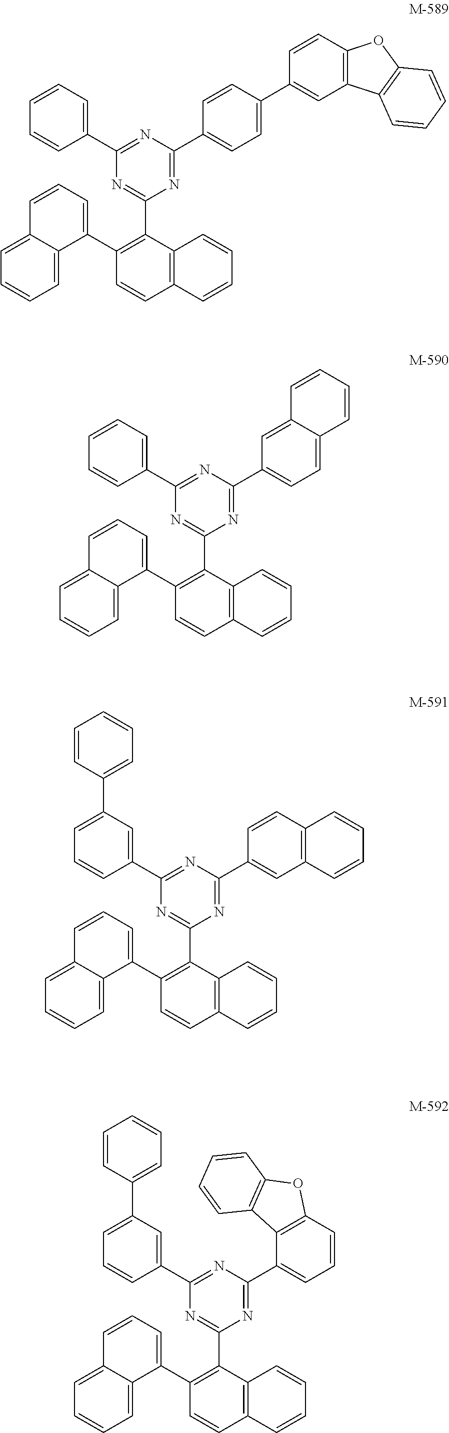 [SMILES: c1ccc(-c2nc(-c3ccc(-c4ccc5oc6ccccc6c5c4)cc3)nc(-c3c(-c4cccc5ccccc45)ccc4ccccc34)n2)cc1 ; c1ccc(-c2nc(-c3ccc4ccccc4c3)nc(-c3c(-c4cccc5ccccc45)ccc4ccccc34)n2)cc1 ; c1ccc(-c2cccc(-c3nc(-c4ccc5ccccc5c4)nc(-c4c(-c5cccc6ccccc56)ccc5ccccc45)n3)c2)cc1 ; c1ccc(-c2cccc(-c3nc(-c4c(-c5cccc6ccccc56)ccc5ccccc45)nc(-c4cccc5oc6ccccc6c45)n3)c2)cc1]

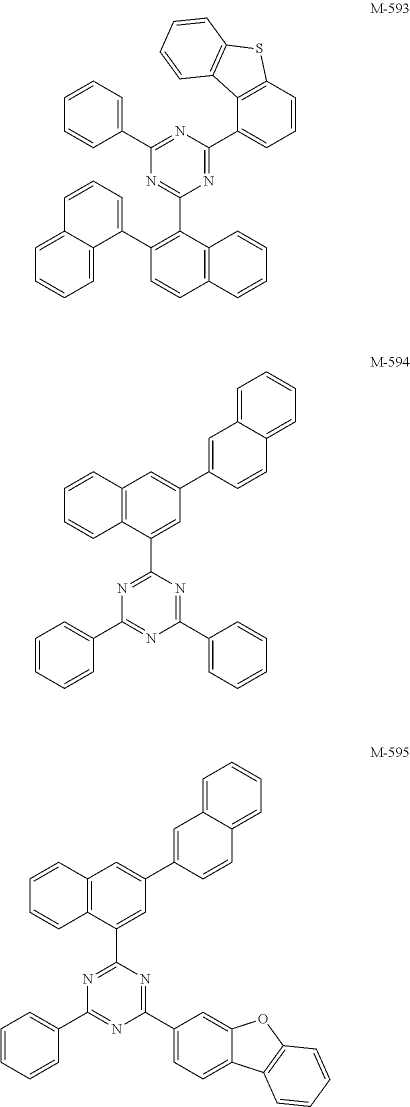 [SMILES: c1ccc(-c2nc(-c3c(-c4cccc5ccccc45)ccc4ccccc34)nc(-c3cccc4sc5ccccc5c34)n2)cc1 ; c1ccc(-c2nc(-c3ccccc3)nc(-c3cc(-c4ccc5ccccc5c4)cc4ccccc34)n2)cc1 ; c1ccc(-c2nc(-c3ccc4c(c3)oc3ccccc34)nc(-c3cc(-c4ccc5ccccc5c4)cc4ccccc34)n2)cc1]

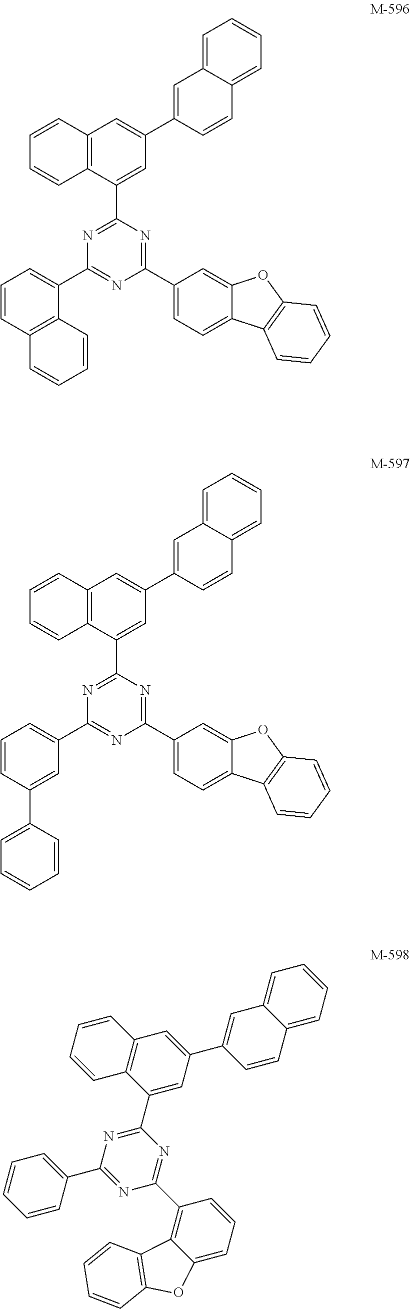 [SMILES: c1ccc2cc(-c3cc(-c4nc(-c5ccc6c(c5)oc5ccccc56)nc(-c5cccc6ccccc56)n4)c4ccccc4c3)ccc2c1 ; c1ccc(-c2cccc(-c3nc(-c4ccc5c(c4)oc4ccccc45)nc(-c4cc(-c5ccc6ccccc6c5)cc5ccccc45)n3)c2)cc1 ; c1ccc(-c2nc(-c3cc(-c4ccc5ccccc5c4)cc4ccccc34)nc(-c3cccc4oc5ccccc5c34)n2)cc1]

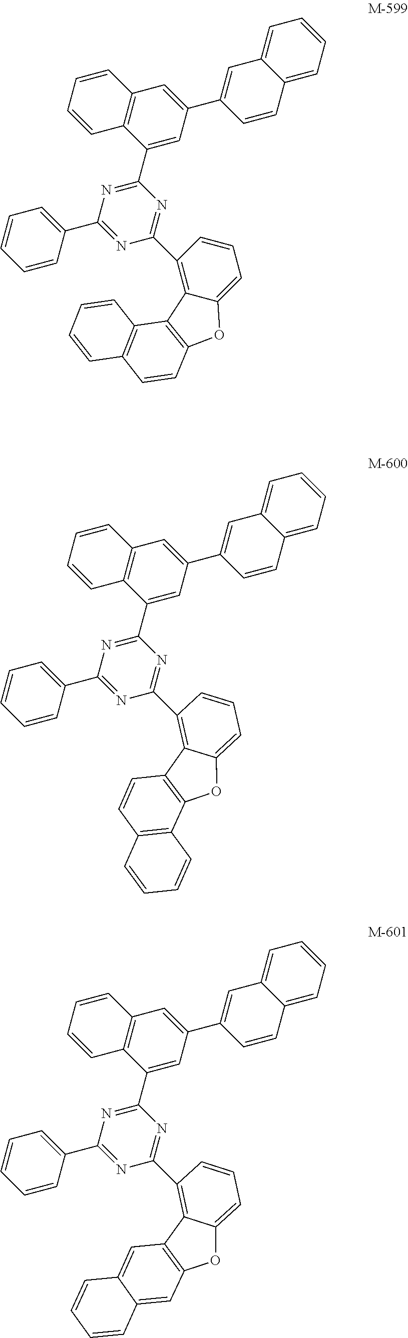 [SMILES: c1ccc(-c2nc(-c3cc(-c4ccc5ccccc5c4)cc4ccccc34)nc(-c3cccc4oc5ccc6ccccc6c5c34)n2)cc1 ; c1ccc(-c2nc(-c3cc(-c4ccc5ccccc5c4)cc4ccccc34)nc(-c3cccc4oc5c6ccccc6ccc5c34)n2)cc1 ; c1ccc(-c2nc(-c3cc(-c4ccc5ccccc5c4)cc4ccccc34)nc(-c3cccc4oc5cc6ccccc6cc5c34)n2)cc1]

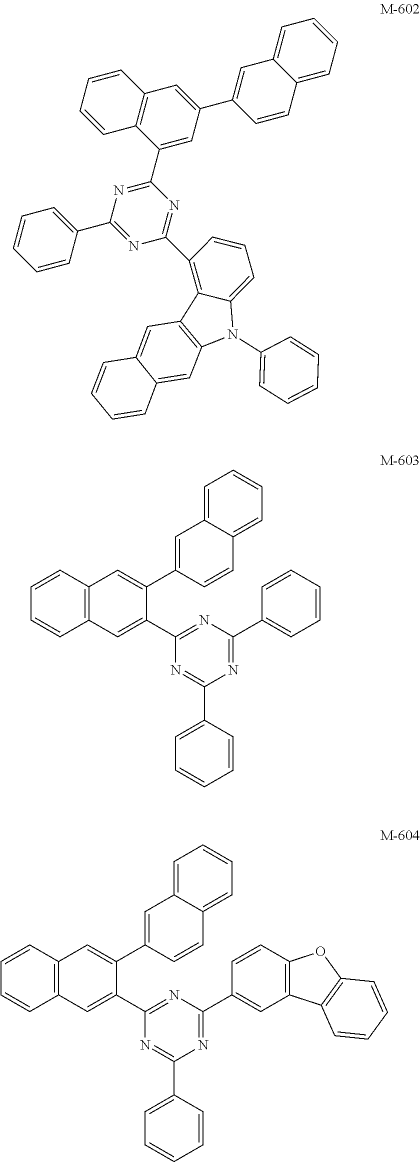 [SMILES: c1ccc(-c2nc(-c3cc(-c4ccc5ccccc5c4)cc4ccccc34)nc(-c3cccc4c3c3cc5ccccc5cc3n4-c3ccccc3)n2)cc1 ; c1ccc(-c2nc(-c3ccccc3)nc(-c3cc4ccccc4cc3-c3ccc4ccccc4c3)n2)cc1 ; c1ccc(-c2nc(-c3ccc4oc5ccccc5c4c3)nc(-c3cc4ccccc4cc3-c3ccc4ccccc4c3)n2)cc1]

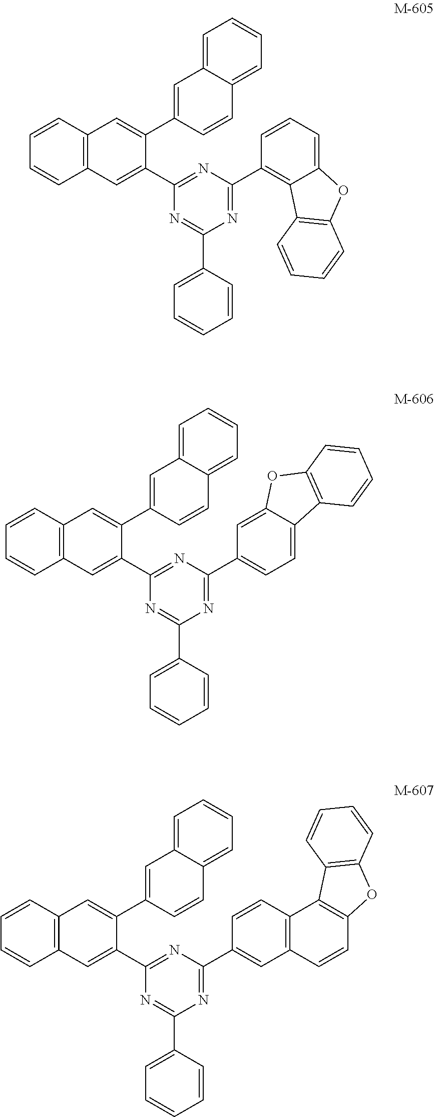 [SMILES: c1ccc(-c2nc(-c3cc4ccccc4cc3-c3ccc4ccccc4c3)nc(-c3cccc4oc5ccccc5c34)n2)cc1 ; c1ccc(-c2nc(-c3ccc4c(c3)oc3ccccc34)nc(-c3cc4ccccc4cc3-c3ccc4ccccc4c3)n2)cc1 ; c1ccc(-c2nc(-c3ccc4c(ccc5oc6ccccc6c54)c3)nc(-c3cc4ccccc4cc3-c3ccc4ccccc4c3)n2)cc1]

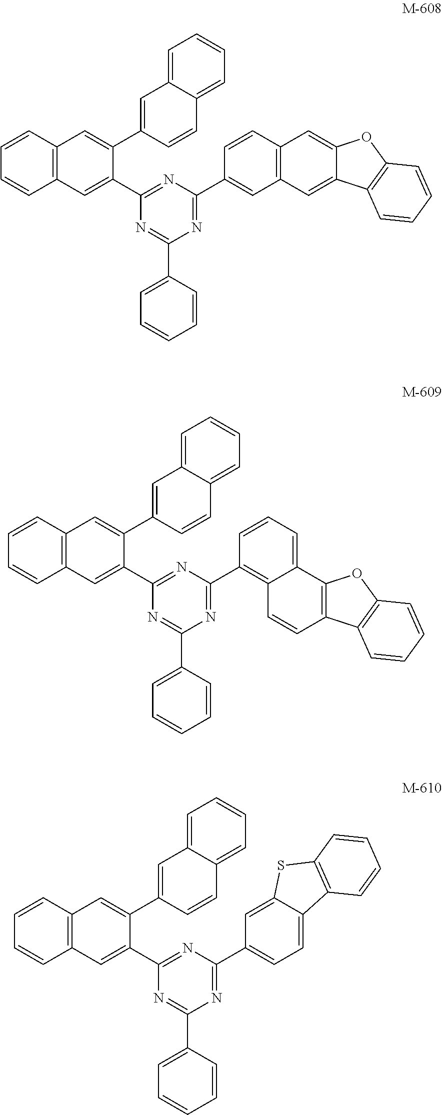 [SMILES: c1ccc(-c2nc(-c3ccc4cc5oc6ccccc6c5cc4c3)nc(-c3cc4ccccc4cc3-c3ccc4ccccc4c3)n2)cc1 ; c1ccc(-c2nc(-c3cc4ccccc4cc3-c3ccc4ccccc4c3)nc(-c3cccc4c3ccc3c5ccccc5oc43)n2)cc1 ; c1ccc(-c2nc(-c3ccc4c(c3)sc3ccccc34)nc(-c3cc4ccccc4cc3-c3ccc4ccccc4c3)n2)cc1]

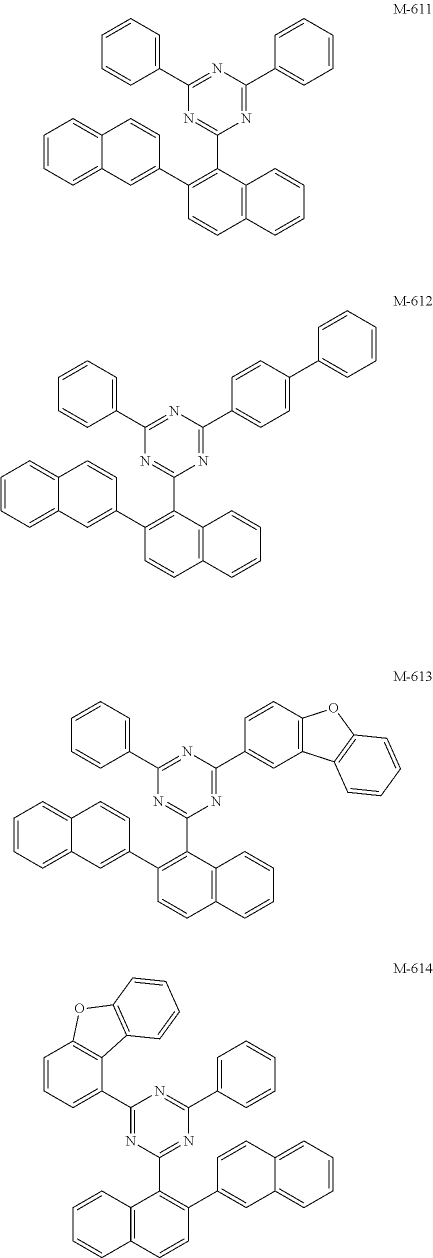 [SMILES: c1ccc(-c2nc(-c3ccccc3)nc(-c3c(-c4ccc5ccccc5c4)ccc4ccccc34)n2)cc1 ; c1ccc(-c2ccc(-c3nc(-c4ccccc4)nc(-c4c(-c5ccc6ccccc6c5)ccc5ccccc45)n3)cc2)cc1 ; c1ccc(-c2nc(-c3ccc4oc5ccccc5c4c3)nc(-c3c(-c4ccc5ccccc5c4)ccc4ccccc34)n2)cc1 ; c1ccc(-c2nc(-c3c(-c4ccc5ccccc5c4)ccc4ccccc34)nc(-c3cccc4oc5ccccc5c34)n2)cc1]

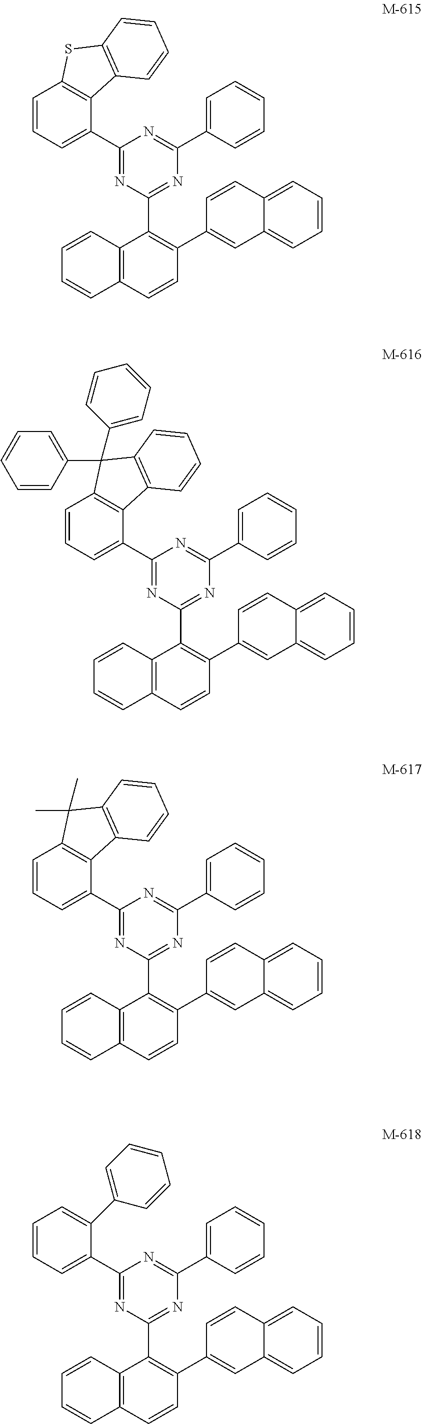 [SMILES: c1ccc(-c2nc(-c3c(-c4ccc5ccccc5c4)ccc4ccccc34)nc(-c3cccc4sc5ccccc5c34)n2)cc1 ; c1ccc(-c2nc(-c3cccc4c3-c3ccccc3C4(c3ccccc3)c3ccccc3)nc(-c3c(-c4ccc5ccccc5c4)ccc4ccccc34)n2)cc1 ; CC1(C)c2ccccc2-c2c(-c3nc(-c4ccccc4)nc(-c4c(-c5ccc6ccccc6c5)ccc5ccccc45)n3)cccc21 ; c1ccc(-c2nc(-c3ccccc3-c3ccccc3)nc(-c3c(-c4ccc5ccccc5c4)ccc4ccccc34)n2)cc1]

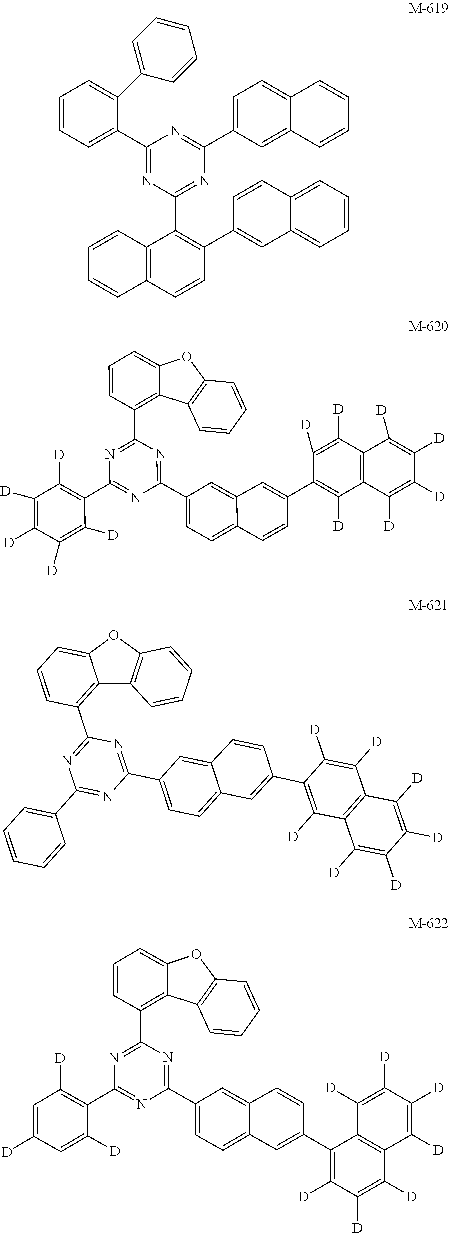 [SMILES: c1ccc(-c2ccccc2-c2nc(-c3ccc4ccccc4c3)nc(-c3c(-c4ccc5ccccc5c4)ccc4ccccc34)n2)cc1 ; [2H]c1c([2H])c([2H])c(-c2nc(-c3ccc4ccc(-c5c([2H])c([2H])c6c([2H])c([2H])c([2H])c([2H])c6c5[2H])cc4c3)nc(-c3cccc4oc5ccccc5c34)n2)c([2H])c1[2H] ; [2H]c1c([2H])c([2H])c2c([2H])c(-c3ccc4cc(-c5nc(-c6ccccc6)nc(-c6cccc7oc8ccccc8c67)n5)ccc4c3)c([2H])c([2H])c2c1[2H] ; [2H]c1cc([2H])c(-c2nc(-c3ccc4cc(-c5c([2H])c([2H])c([2H])c6c([2H])c([2H])c([2H])c([2H])c56)ccc4c3)nc(-c3cccc4oc5ccccc5c34)n2)c([2H])c1]

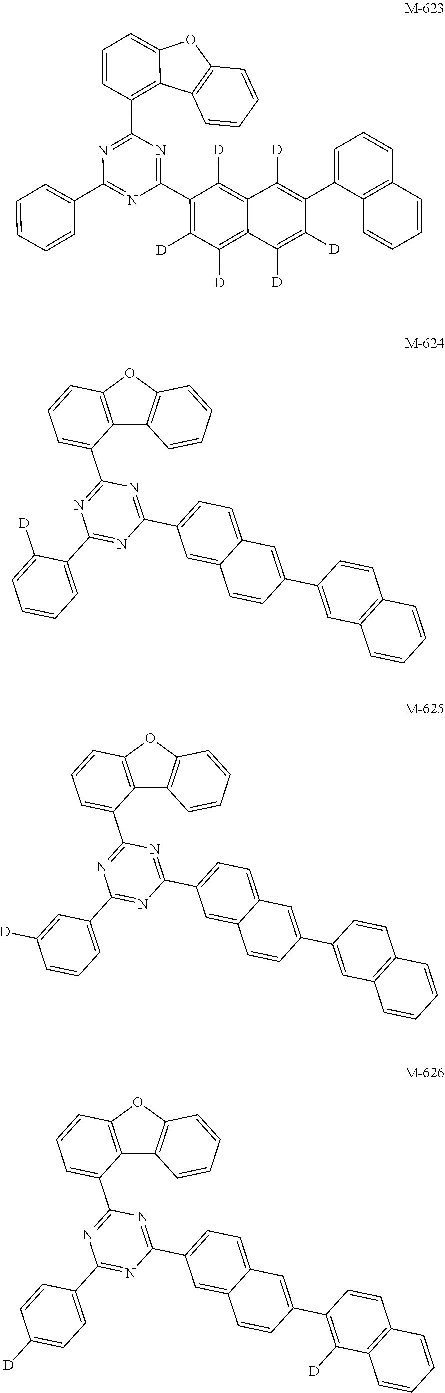 [SMILES: [2H]c1c(-c2nc(-c3ccccc3)nc(-c3cccc4oc5ccccc5c34)n2)c([2H])c2c([2H])c(-c3cccc4ccccc34)c([2H])c([2H])c2c1[2H] ; [2H]c1ccccc1-c1nc(-c2ccc3cc(-c4ccc5ccccc5c4)ccc3c2)nc(-c2cccc3oc4ccccc4c23)n1 ; [2H]c1cccc(-c2nc(-c3ccc4cc(-c5ccc6ccccc6c5)ccc4c3)nc(-c3cccc4oc5ccccc5c34)n2)c1 ; [2H]c1ccc(-c2nc(-c3ccc4cc(-c5ccc6ccccc6c5[2H])ccc4c3)nc(-c3cccc4oc5ccccc5c34)n2)cc1]

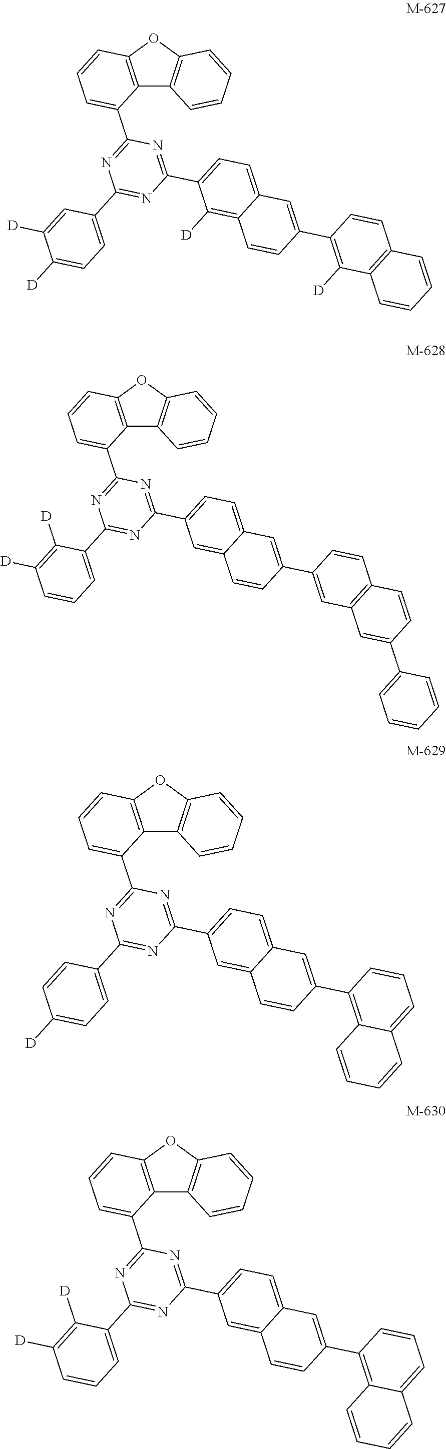 [SMILES: [2H]c1ccc(-c2nc(-c3ccc4cc(-c5ccc6ccccc6c5[2H])ccc4c3[2H])nc(-c3cccc4oc5ccccc5c34)n2)cc1[2H] ; [2H]c1cccc(-c2nc(-c3ccc4cc(-c5ccc6ccc(-c7ccccc7)cc6c5)ccc4c3)nc(-c3cccc4oc5ccccc5c34)n2)c1[2H] ; [2H]c1ccc(-c2nc(-c3ccc4cc(-c5cccc6ccccc56)ccc4c3)nc(-c3cccc4oc5ccccc5c34)n2)cc1 ; [2H]c1cccc(-c2nc(-c3ccc4cc(-c5cccc6ccccc56)ccc4c3)nc(-c3cccc4oc5ccccc5c34)n2)c1[2H]]

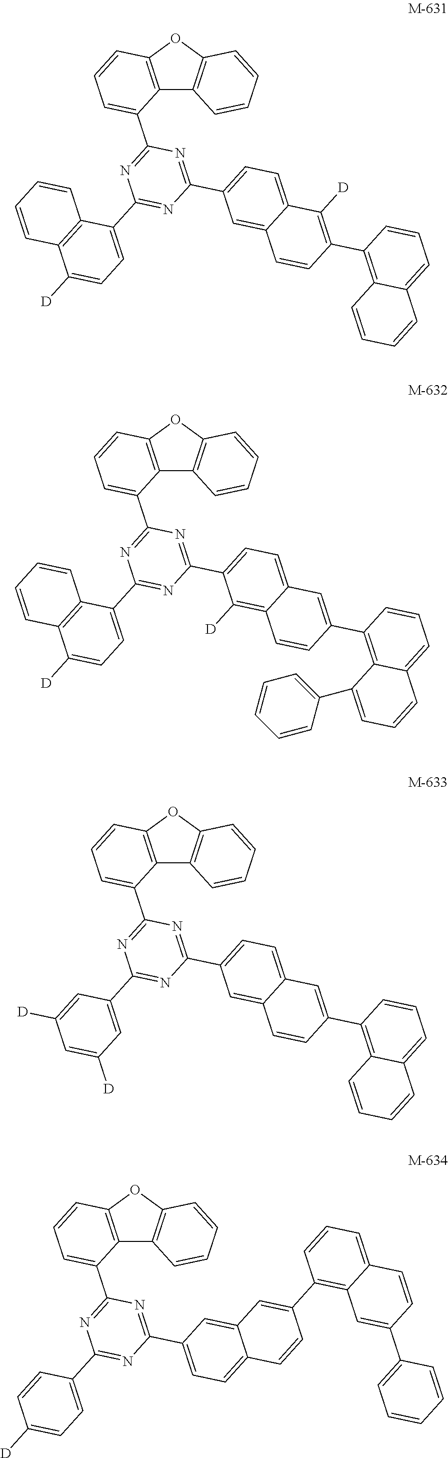 [SMILES: [2H]c1c(-c2cccc3ccccc23)ccc2cc(-c3nc(-c4ccc([2H])c5ccccc45)nc(-c4cccc5oc6ccccc6c45)n3)ccc12 ; [2H]c1c(-c2nc(-c3ccc([2H])c4ccccc34)nc(-c3cccc4oc5ccccc5c34)n2)ccc2cc(-c3cccc4cccc(-c5ccccc5)c34)ccc12 ; [2H]c1cc([2H])cc(-c2nc(-c3ccc4cc(-c5cccc6ccccc56)ccc4c3)nc(-c3cccc4oc5ccccc5c34)n2)c1 ; [2H]c1ccc(-c2nc(-c3ccc4ccc(-c5cccc6ccc(-c7ccccc7)cc56)cc4c3)nc(-c3cccc4oc5ccccc5c34)n2)cc1]

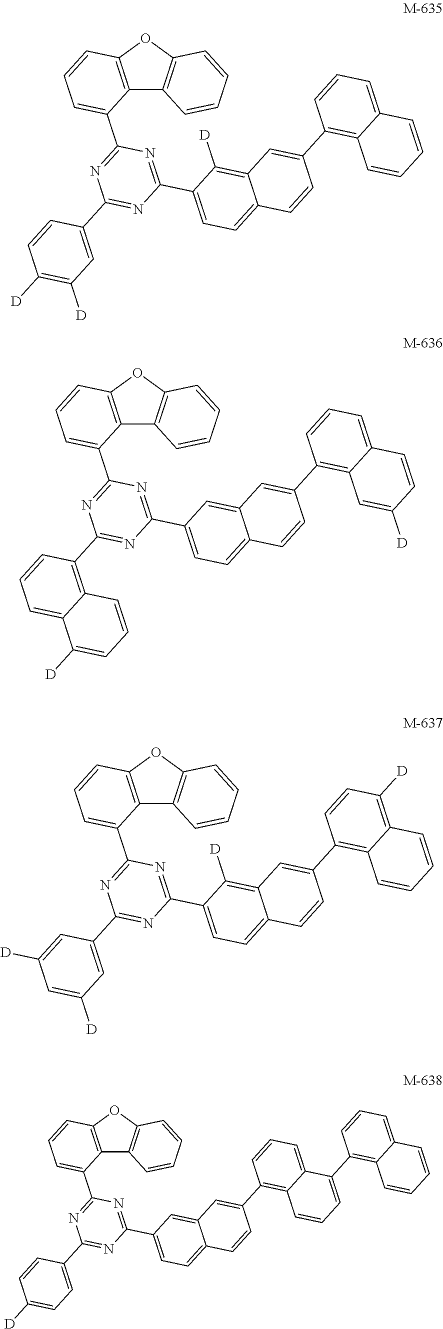 [SMILES: [2H]c1ccc(-c2nc(-c3ccc4ccc(-c5cccc6ccccc56)cc4c3[2H])nc(-c3cccc4oc5ccccc5c34)n2)cc1[2H] ; [2H]c1ccc2cccc(-c3ccc4ccc(-c5nc(-c6cccc7c([2H])cccc67)nc(-c6cccc7oc8ccccc8c67)n5)cc4c3)c2c1 ; [2H]c1cc([2H])cc(-c2nc(-c3ccc4ccc(-c5ccc([2H])c6ccccc56)cc4c3[2H])nc(-c3cccc4oc5ccccc5c34)n2)c1 ; [2H]c1ccc(-c2nc(-c3ccc4ccc(-c5cccc6c(-c7cccc8ccccc78)cccc56)cc4c3)nc(-c3cccc4oc5ccccc5c34)n2)cc1]

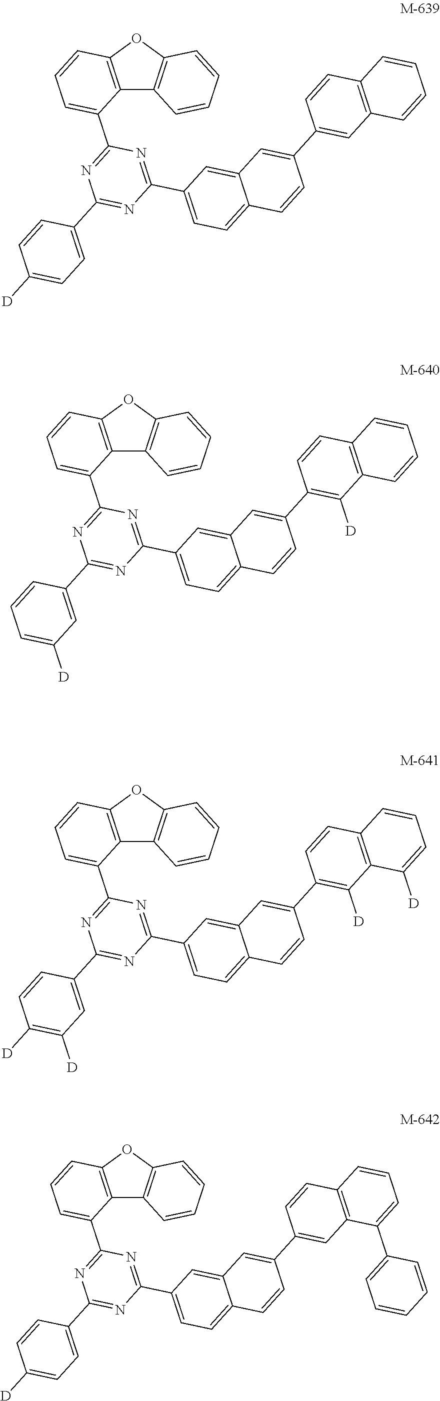 [SMILES: [2H]c1ccc(-c2nc(-c3ccc4ccc(-c5ccc6ccccc6c5)cc4c3)nc(-c3cccc4oc5ccccc5c34)n2)cc1 ; [2H]c1cccc(-c2nc(-c3ccc4ccc(-c5ccc6ccccc6c5[2H])cc4c3)nc(-c3cccc4oc5ccccc5c34)n2)c1 ; [2H]c1ccc(-c2nc(-c3ccc4ccc(-c5ccc6cccc([2H])c6c5[2H])cc4c3)nc(-c3cccc4oc5ccccc5c34)n2)cc1[2H] ; [2H]c1ccc(-c2nc(-c3ccc4ccc(-c5ccc6cccc(-c7ccccc7)c6c5)cc4c3)nc(-c3cccc4oc5ccccc5c34)n2)cc1]

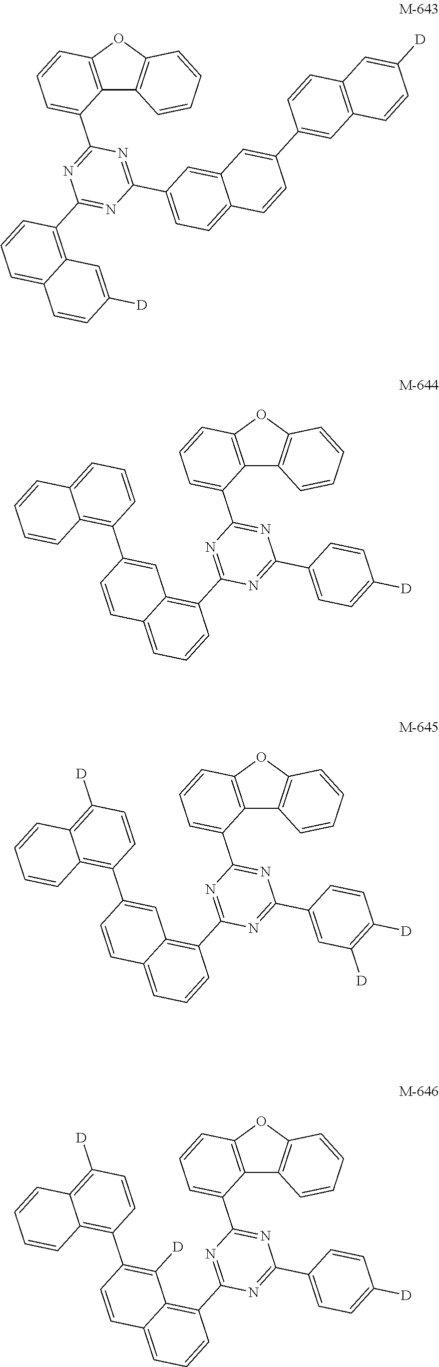 [SMILES: [2H]c1ccc2cc(-c3ccc4ccc(-c5nc(-c6cccc7ccc([2H])cc67)nc(-c6cccc7oc8ccccc8c67)n5)cc4c3)ccc2c1 ; [2H]c1ccc(-c2nc(-c3cccc4ccc(-c5cccc6ccccc56)cc34)nc(-c3cccc4oc5ccccc5c34)n2)cc1 ; [2H]c1ccc(-c2nc(-c3cccc4ccc(-c5ccc([2H])c6ccccc56)cc34)nc(-c3cccc4oc5ccccc5c34)n2)cc1[2H] ; [2H]c1ccc(-c2nc(-c3cccc4ccc(-c5ccc([2H])c6ccccc56)c([2H])c34)nc(-c3cccc4oc5ccccc5c34)n2)cc1]

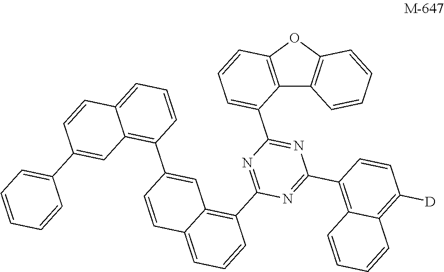 [SMILES: [2H]c1ccc(-c2nc(-c3cccc4ccc(-c5cccc6ccc(-c7ccccc7)cc56)cc34)nc(-c3cccc4oc5ccccc5c34)n2)c2ccccc12]

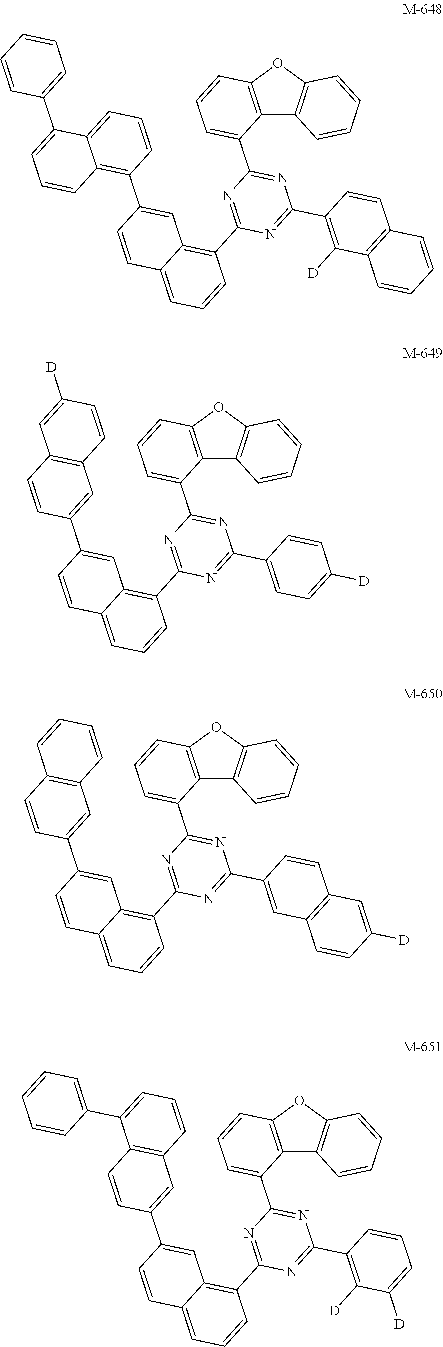 [SMILES: [2H]c1c(-c2nc(-c3cccc4ccc(-c5cccc6c(-c7ccccc7)cccc56)cc34)nc(-c3cccc4oc5ccccc5c34)n2)ccc2ccccc12 ; [2H]c1ccc(-c2nc(-c3cccc4ccc(-c5ccc6cc([2H])ccc6c5)cc34)nc(-c3cccc4oc5ccccc5c34)n2)cc1 ; [2H]c1ccc2cc(-c3nc(-c4cccc5ccc(-c6ccc7ccccc7c6)cc45)nc(-c4cccc5oc6ccccc6c45)n3)ccc2c1 ; [2H]c1cccc(-c2nc(-c3cccc4ccc(-c5ccc6c(-c7ccccc7)cccc6c5)cc34)nc(-c3cccc4oc5ccccc5c34)n2)c1[2H]]

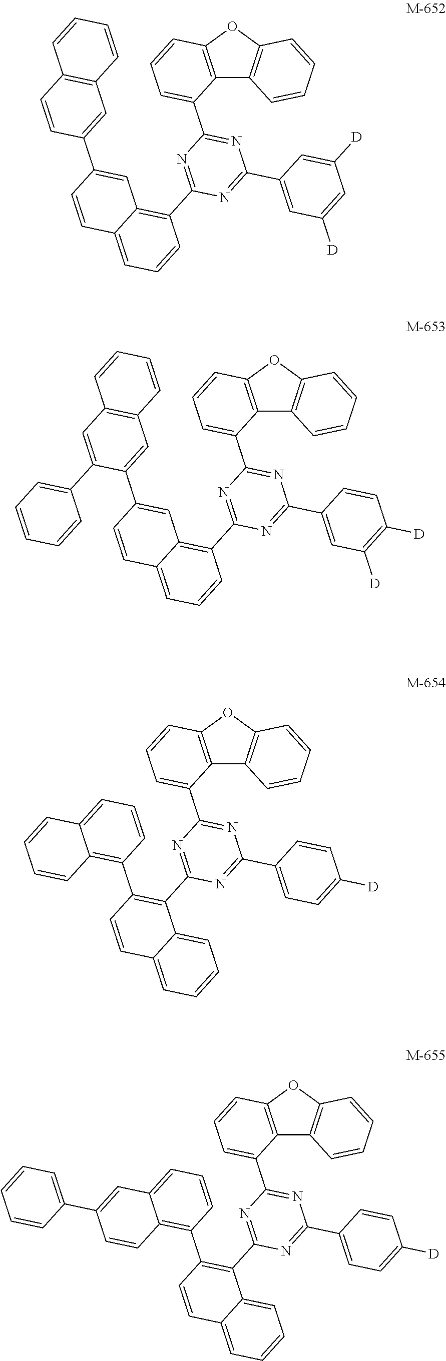 [SMILES: [2H]c1cc([2H])cc(-c2nc(-c3cccc4ccc(-c5ccc6ccccc6c5)cc34)nc(-c3cccc4oc5ccccc5c34)n2)c1 ; [2H]c1ccc(-c2nc(-c3cccc4ccc(-c5cc6ccccc6cc5-c5ccccc5)cc34)nc(-c3cccc4oc5ccccc5c34)n2)cc1[2H] ; [2H]c1ccc(-c2nc(-c3c(-c4cccc5ccccc45)ccc4ccccc34)nc(-c3cccc4oc5ccccc5c34)n2)cc1 ; [2H]c1ccc(-c2nc(-c3c(-c4cccc5cc(-c6ccccc6)ccc45)ccc4ccccc34)nc(-c3cccc4oc5ccccc5c34)n2)cc1]

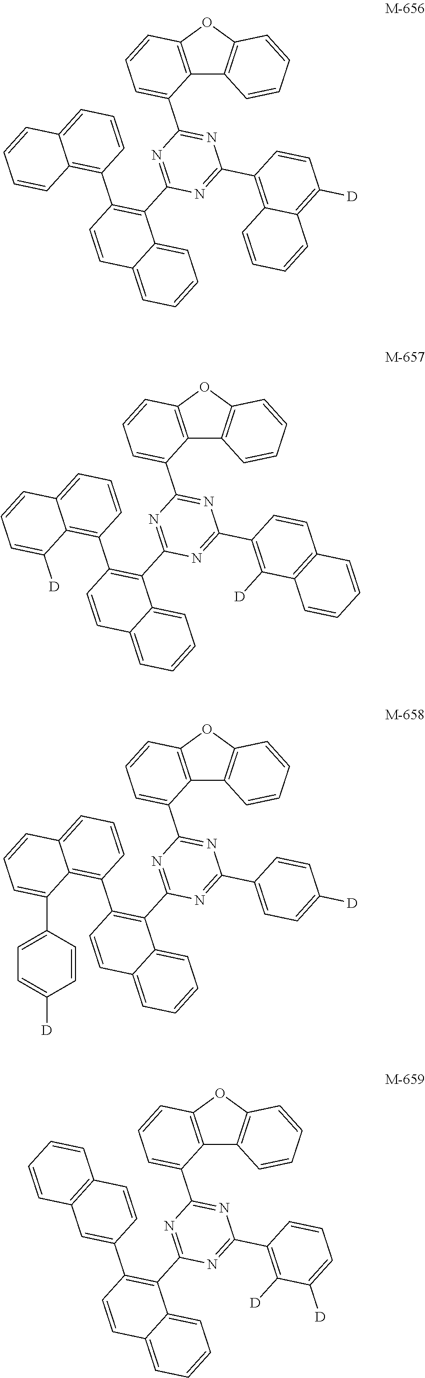 [SMILES: [2H]c1ccc(-c2nc(-c3c(-c4cccc5ccccc45)ccc4ccccc34)nc(-c3cccc4oc5ccccc5c34)n2)c2ccccc12 ; [2H]c1c(-c2nc(-c3c(-c4cccc5cccc([2H])c45)ccc4ccccc34)nc(-c3cccc4oc5ccccc5c34)n2)ccc2ccccc12 ; [2H]c1ccc(-c2nc(-c3c(-c4cccc5cccc(-c6ccc([2H])cc6)c45)ccc4ccccc34)nc(-c3cccc4oc5ccccc5c34)n2)cc1 ; [2H]c1cccc(-c2nc(-c3c(-c4ccc5ccccc5c4)ccc4ccccc34)nc(-c3cccc4oc5ccccc5c34)n2)c1[2H]]

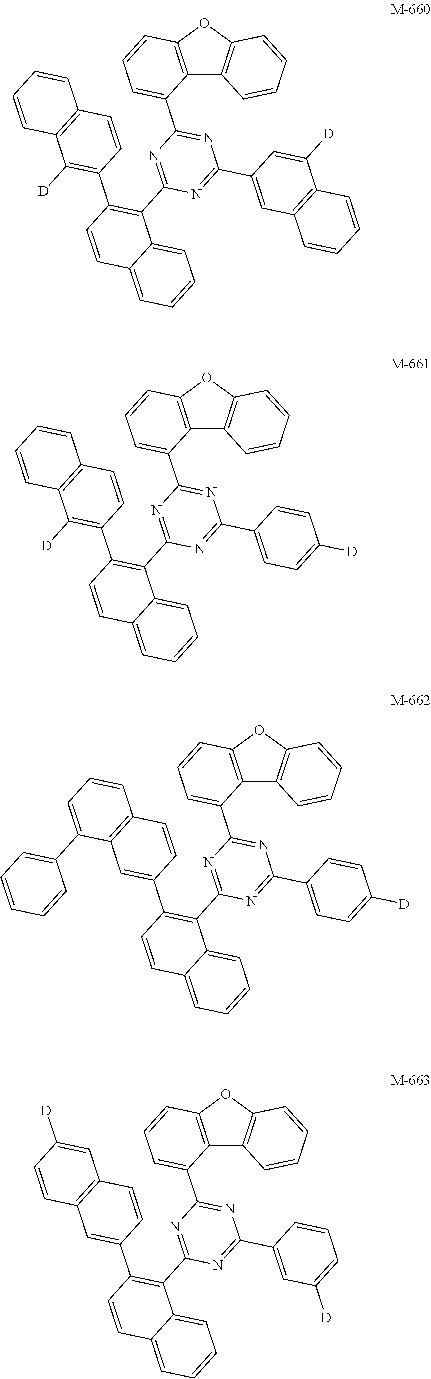 [SMILES: [2H]c1cc(-c2nc(-c3c(-c4ccc5ccccc5c4[2H])ccc4ccccc34)nc(-c3cccc4oc5ccccc5c34)n2)cc2ccccc12 ; [2H]c1ccc(-c2nc(-c3c(-c4ccc5ccccc5c4[2H])ccc4ccccc34)nc(-c3cccc4oc5ccccc5c34)n2)cc1 ; [2H]c1ccc(-c2nc(-c3c(-c4ccc5cccc(-c6ccccc6)c5c4)ccc4ccccc34)nc(-c3cccc4oc5ccccc5c34)n2)cc1 ; [2H]c1cccc(-c2nc(-c3c(-c4ccc5cc([2H])ccc5c4)ccc4ccccc34)nc(-c3cccc4oc5ccccc5c34)n2)c1]

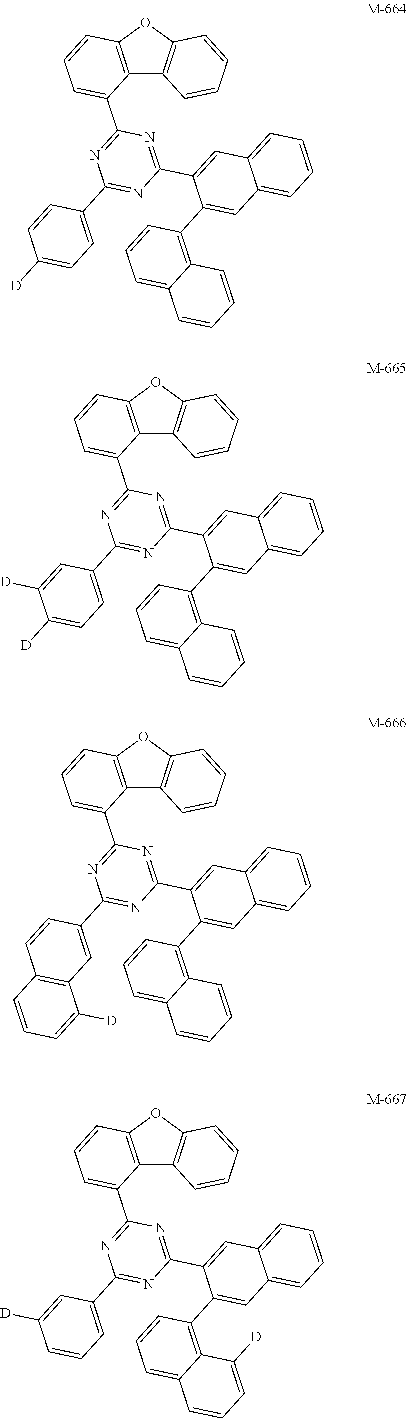 [SMILES: [2H]c1ccc(-c2nc(-c3cc4ccccc4cc3-c3cccc4ccccc34)nc(-c3cccc4oc5ccccc5c34)n2)cc1 ; [2H]c1ccc(-c2nc(-c3cc4ccccc4cc3-c3cccc4ccccc34)nc(-c3cccc4oc5ccccc5c34)n2)cc1[2H] ; [2H]c1cccc2ccc(-c3nc(-c4cc5ccccc5cc4-c4cccc5ccccc45)nc(-c4cccc5oc6ccccc6c45)n3)cc12 ; [2H]c1cccc(-c2nc(-c3cc4ccccc4cc3-c3cccc4cccc([2H])c34)nc(-c3cccc4oc5ccccc5c34)n2)c1]

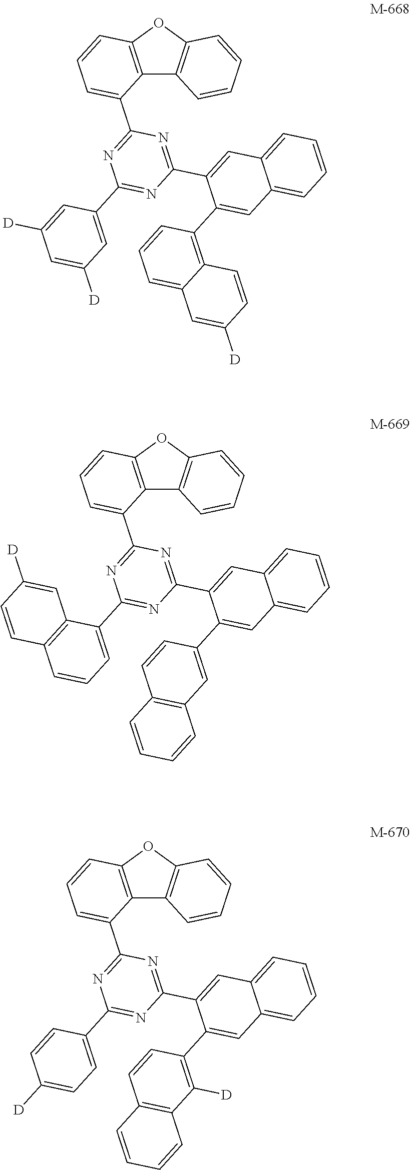 [SMILES: [2H]c1cc([2H])cc(-c2nc(-c3cc4ccccc4cc3-c3cccc4cc([2H])ccc34)nc(-c3cccc4oc5ccccc5c34)n2)c1 ; [2H]c1ccc2cccc(-c3nc(-c4cc5ccccc5cc4-c4ccc5ccccc5c4)nc(-c4cccc5oc6ccccc6c45)n3)c2c1 ; [2H]c1ccc(-c2nc(-c3cc4ccccc4cc3-c3ccc4ccccc4c3[2H])nc(-c3cccc4oc5ccccc5c34)n2)cc1]

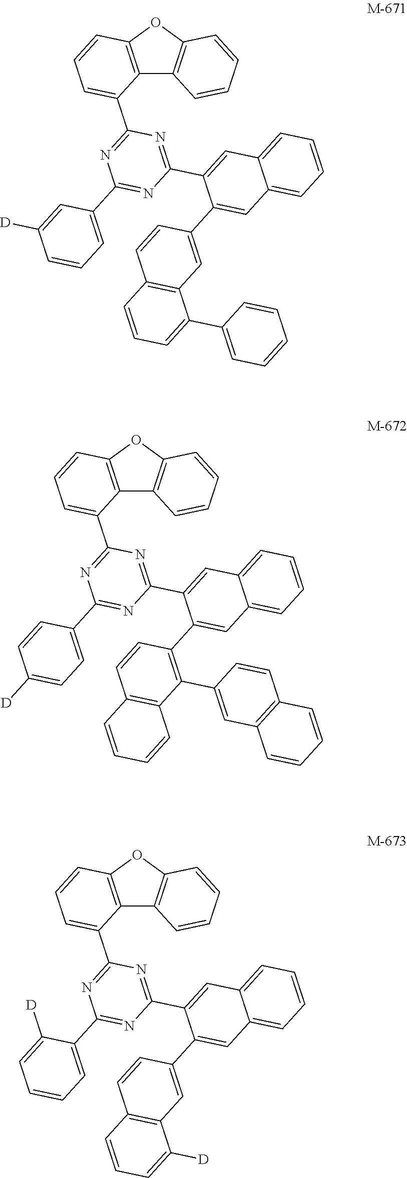 [SMILES: [2H]c1cccc(-c2nc(-c3cc4ccccc4cc3-c3ccc4cccc(-c5ccccc5)c4c3)nc(-c3cccc4oc5ccccc5c34)n2)c1 ; [2H]c1ccc(-c2nc(-c3cc4ccccc4cc3-c3ccc4ccccc4c3-c3ccc4ccccc4c3)nc(-c3cccc4oc5ccccc5c34)n2)cc1 ; [2H]c1ccccc1-c1nc(-c2cc3ccccc3cc2-c2ccc3cccc([2H])c3c2)nc(-c2cccc3oc4ccccc4c23)n1]

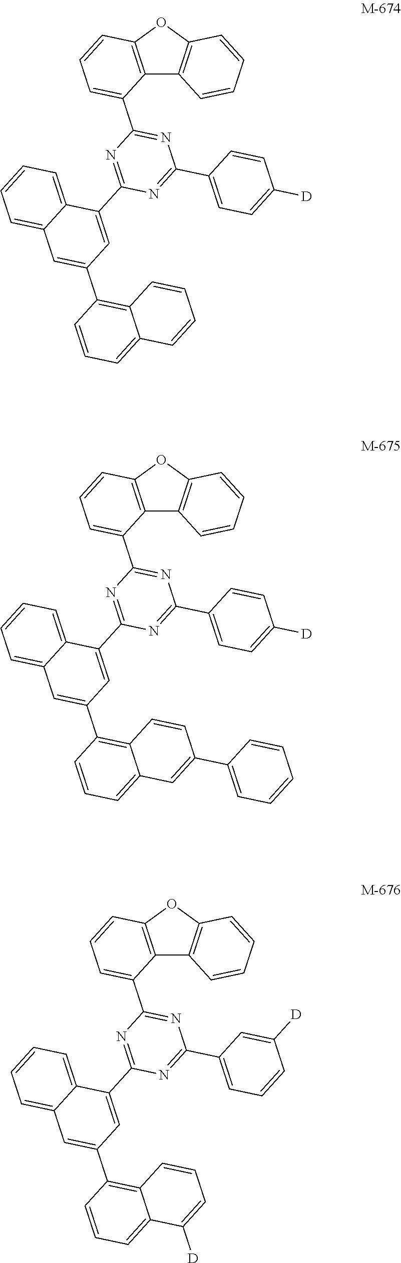 [SMILES: [2H]c1ccc(-c2nc(-c3cc(-c4cccc5ccccc45)cc4ccccc34)nc(-c3cccc4oc5ccccc5c34)n2)cc1 ; [2H]c1ccc(-c2nc(-c3cc(-c4cccc5cc(-c6ccccc6)ccc45)cc4ccccc34)nc(-c3cccc4oc5ccccc5c34)n2)cc1 ; [2H]c1cccc(-c2nc(-c3cc(-c4cccc5c([2H])cccc45)cc4ccccc34)nc(-c3cccc4oc5ccccc5c34)n2)c1]

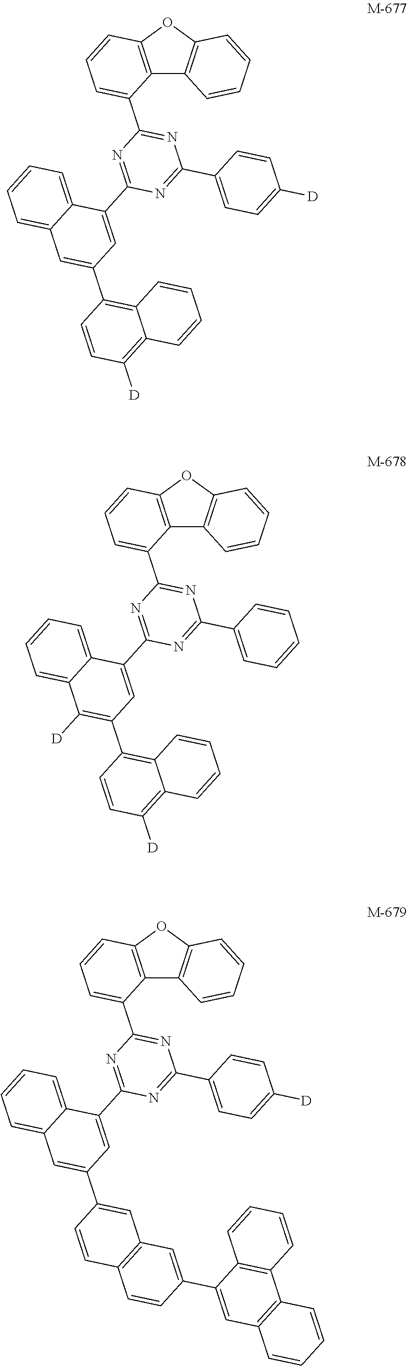 [SMILES: [2H]c1ccc(-c2nc(-c3cc(-c4ccc([2H])c5ccccc45)cc4ccccc34)nc(-c3cccc4oc5ccccc5c34)n2)cc1 ; [2H]c1ccc(-c2cc(-c3nc(-c4ccccc4)nc(-c4cccc5oc6ccccc6c45)n3)c3ccccc3c2[2H])c2ccccc12 ; [2H]c1ccc(-c2nc(-c3cc(-c4ccc5ccc(-c6cc7ccccc7c7ccccc67)cc5c4)cc4ccccc34)nc(-c3cccc4oc5ccccc5c34)n2)cc1]

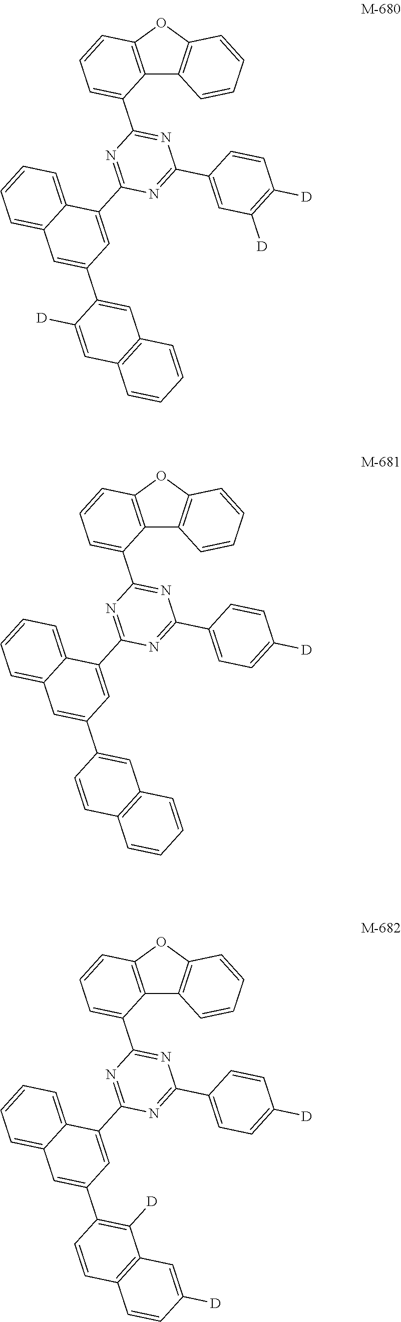 [SMILES: [2H]c1ccc(-c2nc(-c3cc(-c4cc5ccccc5cc4[2H])cc4ccccc34)nc(-c3cccc4oc5ccccc5c34)n2)cc1[2H] ; [2H]c1ccc(-c2nc(-c3cc(-c4ccc5ccccc5c4)cc4ccccc34)nc(-c3cccc4oc5ccccc5c34)n2)cc1 ; [2H]c1ccc(-c2nc(-c3cc(-c4ccc5ccc([2H])cc5c4[2H])cc4ccccc34)nc(-c3cccc4oc5ccccc5c34)n2)cc1]

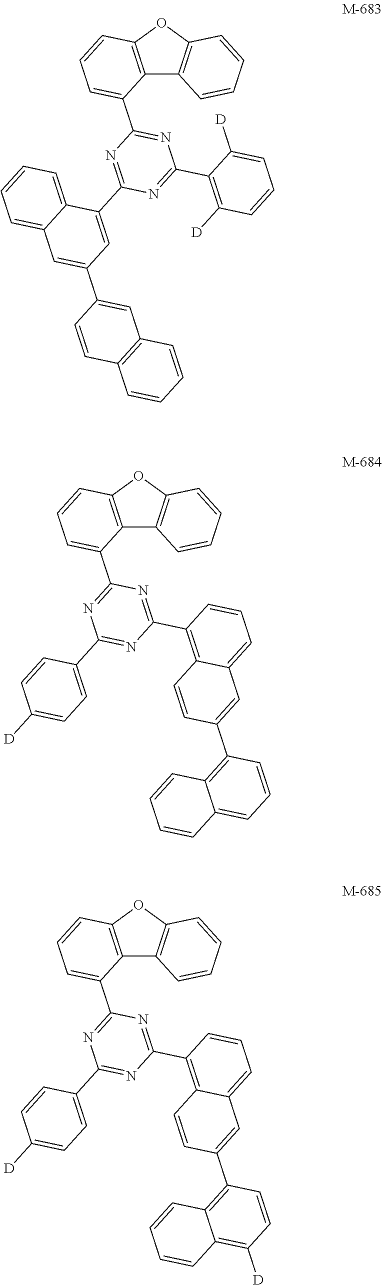 [SMILES: [2H]c1cccc([2H])c1-c1nc(-c2cc(-c3ccc4ccccc4c3)cc3ccccc23)nc(-c2cccc3oc4ccccc4c23)n1 ; [2H]c1ccc(-c2nc(-c3cccc4cc(-c5cccc6ccccc56)ccc34)nc(-c3cccc4oc5ccccc5c34)n2)cc1 ; [2H]c1ccc(-c2nc(-c3cccc4cc(-c5ccc([2H])c6ccccc56)ccc34)nc(-c3cccc4oc5ccccc5c34)n2)cc1]

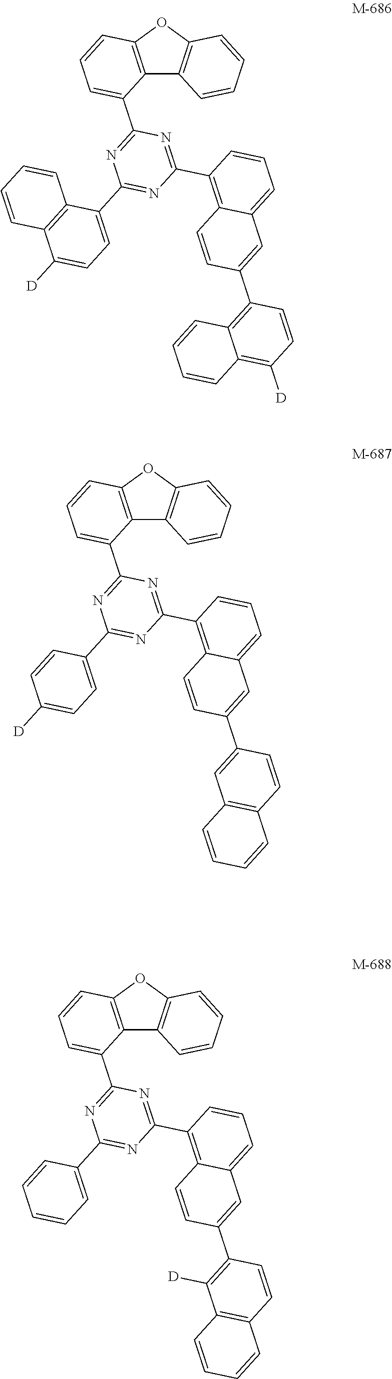 [SMILES: [2H]c1ccc(-c2ccc3c(-c4nc(-c5ccc([2H])c6ccccc56)nc(-c5cccc6oc7ccccc7c56)n4)cccc3c2)c2ccccc12 ; [2H]c1ccc(-c2nc(-c3cccc4cc(-c5ccc6ccccc6c5)ccc34)nc(-c3cccc4oc5ccccc5c34)n2)cc1 ; [2H]c1c(-c2ccc3c(-c4nc(-c5ccccc5)nc(-c5cccc6oc7ccccc7c56)n4)cccc3c2)ccc2ccccc12]

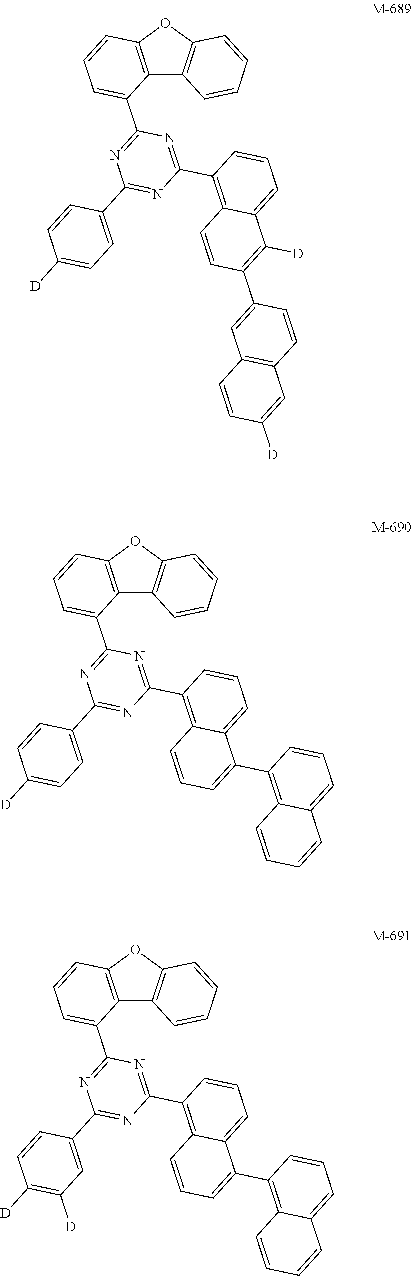 [SMILES: [2H]c1ccc(-c2nc(-c3cccc4c([2H])c(-c5ccc6cc([2H])ccc6c5)ccc34)nc(-c3cccc4oc5ccccc5c34)n2)cc1 ; [2H]c1ccc(-c2nc(-c3cccc4c(-c5cccc6ccccc56)cccc34)nc(-c3cccc4oc5ccccc5c34)n2)cc1 ; [2H]c1ccc(-c2nc(-c3cccc4c(-c5cccc6ccccc56)cccc34)nc(-c3cccc4oc5ccccc5c34)n2)cc1[2H]]

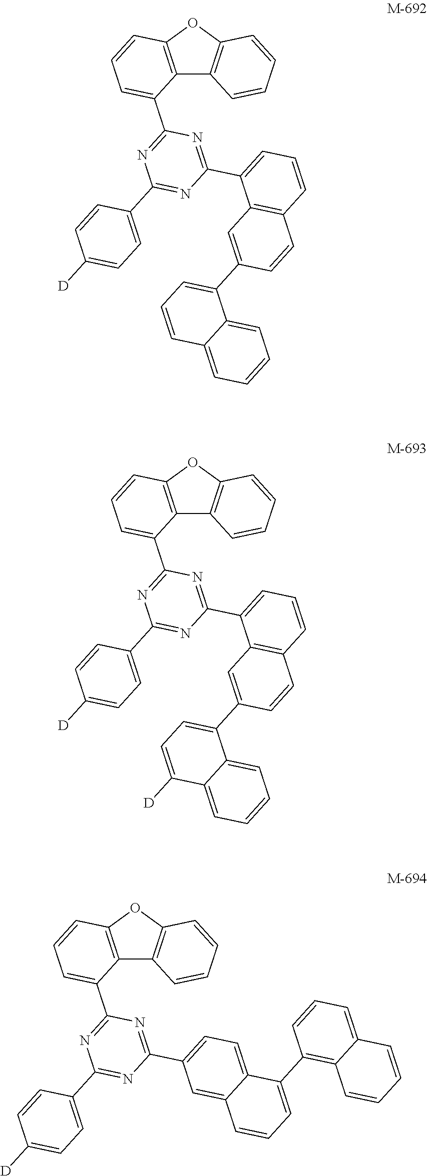 [SMILES: [2H]c1ccc(-c2nc(-c3cccc4ccc(-c5cccc6ccccc56)cc34)nc(-c3cccc4oc5ccccc5c34)n2)cc1 ; [2H]c1ccc(-c2nc(-c3cccc4ccc(-c5ccc([2H])c6ccccc56)cc34)nc(-c3cccc4oc5ccccc5c34)n2)cc1 ; [2H]c1ccc(-c2nc(-c3ccc4c(-c5cccc6ccccc56)cccc4c3)nc(-c3cccc4oc5ccccc5c34)n2)cc1]

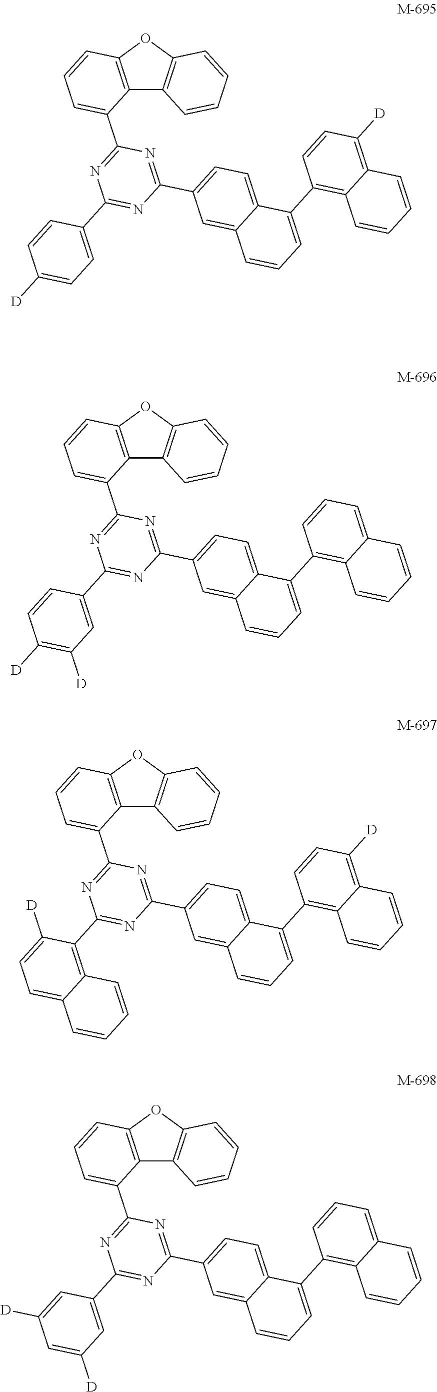 [SMILES: [2H]c1ccc(-c2nc(-c3ccc4c(-c5ccc([2H])c6ccccc56)cccc4c3)nc(-c3cccc4oc5ccccc5c34)n2)cc1 ; [2H]c1ccc(-c2nc(-c3ccc4c(-c5cccc6ccccc56)cccc4c3)nc(-c3cccc4oc5ccccc5c34)n2)cc1[2H] ; [2H]c1ccc2ccccc2c1-c1nc(-c2ccc3c(-c4ccc([2H])c5ccccc45)cccc3c2)nc(-c2cccc3oc4ccccc4c23)n1 ; [2H]c1cc([2H])cc(-c2nc(-c3ccc4c(-c5cccc6ccccc56)cccc4c3)nc(-c3cccc4oc5ccccc5c34)n2)c1]

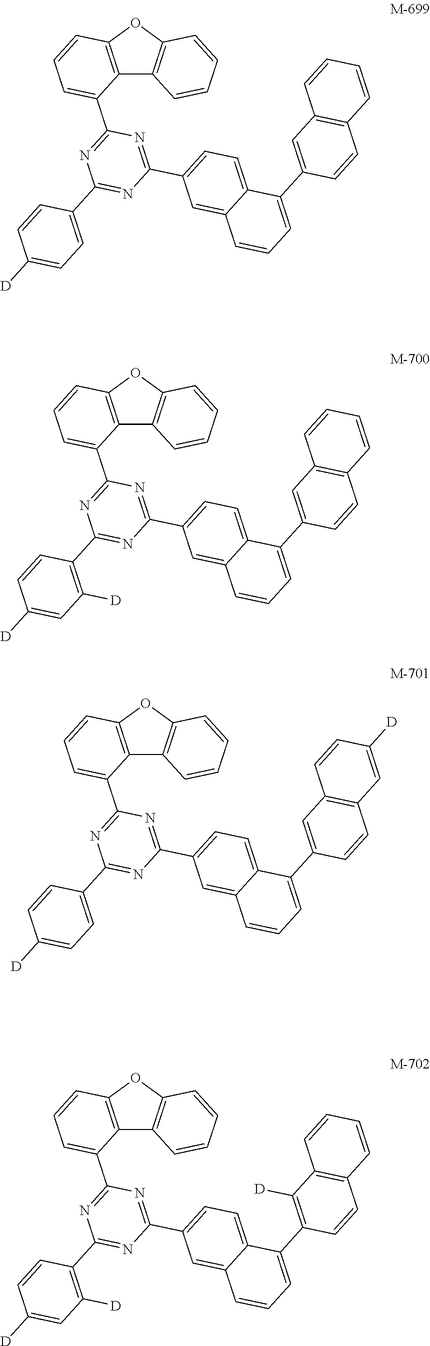 [SMILES: [2H]c1ccc(-c2nc(-c3ccc4c(-c5ccc6ccccc6c5)cccc4c3)nc(-c3cccc4oc5ccccc5c34)n2)cc1 ; [2H]c1ccc(-c2nc(-c3ccc4c(-c5ccc6ccccc6c5)cccc4c3)nc(-c3cccc4oc5ccccc5c34)n2)c([2H])c1 ; [2H]c1ccc(-c2nc(-c3ccc4c(-c5ccc6cc([2H])ccc6c5)cccc4c3)nc(-c3cccc4oc5ccccc5c34)n2)cc1 ; [2H]c1ccc(-c2nc(-c3ccc4c(-c5ccc6ccccc6c5[2H])cccc4c3)nc(-c3cccc4oc5ccccc5c34)n2)c([2H])c1]

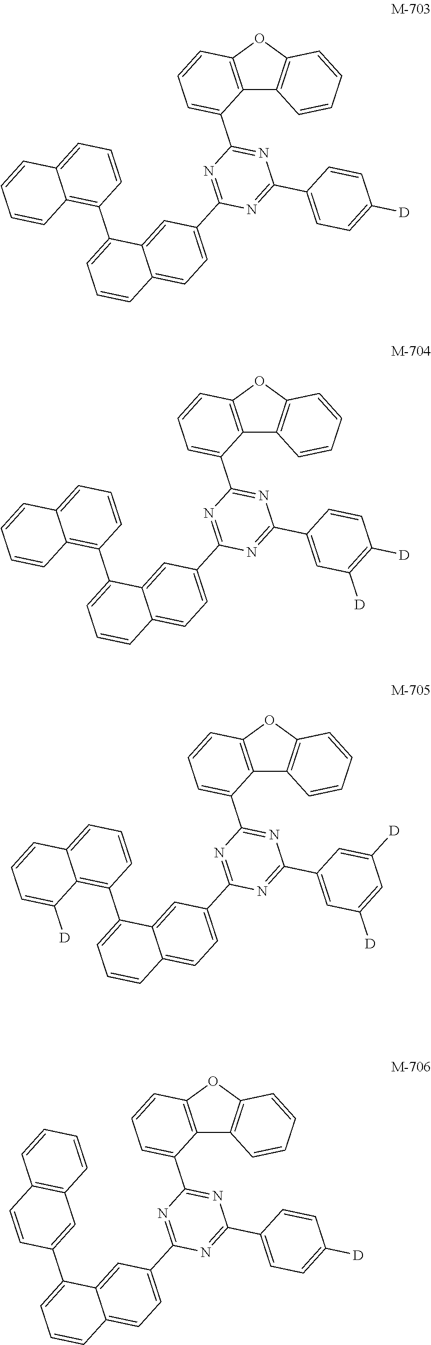 [SMILES: [2H]c1ccc(-c2nc(-c3ccc4cccc(-c5cccc6ccccc56)c4c3)nc(-c3cccc4oc5ccccc5c34)n2)cc1 ; [2H]c1ccc(-c2nc(-c3ccc4cccc(-c5cccc6ccccc56)c4c3)nc(-c3cccc4oc5ccccc5c34)n2)cc1[2H] ; [2H]c1cc([2H])cc(-c2nc(-c3ccc4cccc(-c5cccc6cccc([2H])c56)c4c3)nc(-c3cccc4oc5ccccc5c34)n2)c1 ; [2H]c1ccc(-c2nc(-c3ccc4cccc(-c5ccc6ccccc6c5)c4c3)nc(-c3cccc4oc5ccccc5c34)n2)cc1]

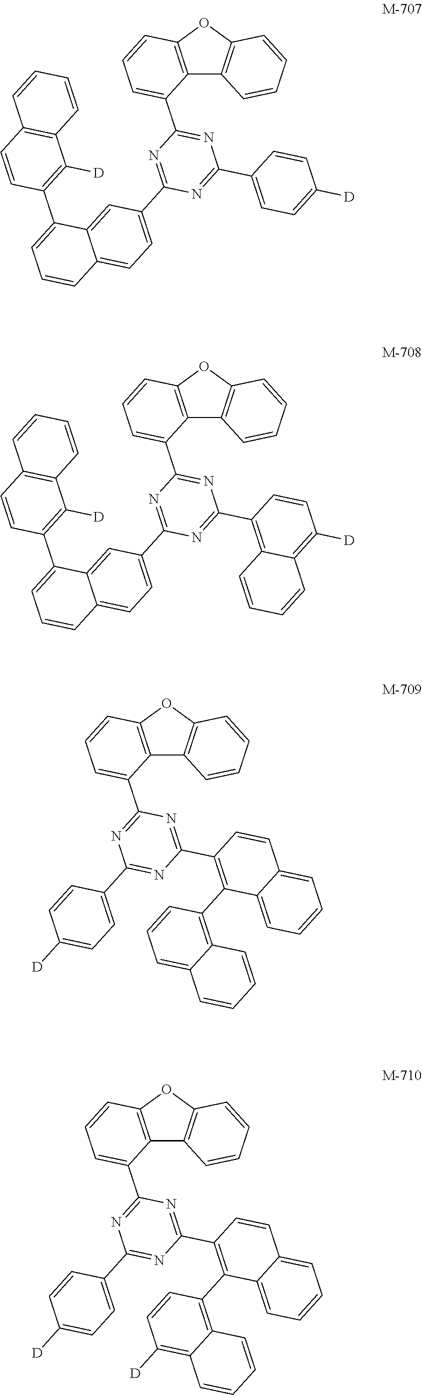 [SMILES: [2H]c1ccc(-c2nc(-c3ccc4cccc(-c5ccc6ccccc6c5[2H])c4c3)nc(-c3cccc4oc5ccccc5c34)n2)cc1 ; [2H]c1c(-c2cccc3ccc(-c4nc(-c5ccc([2H])c6ccccc56)nc(-c5cccc6oc7ccccc7c56)n4)cc23)ccc2ccccc12 ; [2H]c1ccc(-c2nc(-c3ccc4ccccc4c3-c3cccc4ccccc34)nc(-c3cccc4oc5ccccc5c34)n2)cc1 ; [2H]c1ccc(-c2nc(-c3ccc4ccccc4c3-c3ccc([2H])c4ccccc34)nc(-c3cccc4oc5ccccc5c34)n2)cc1]

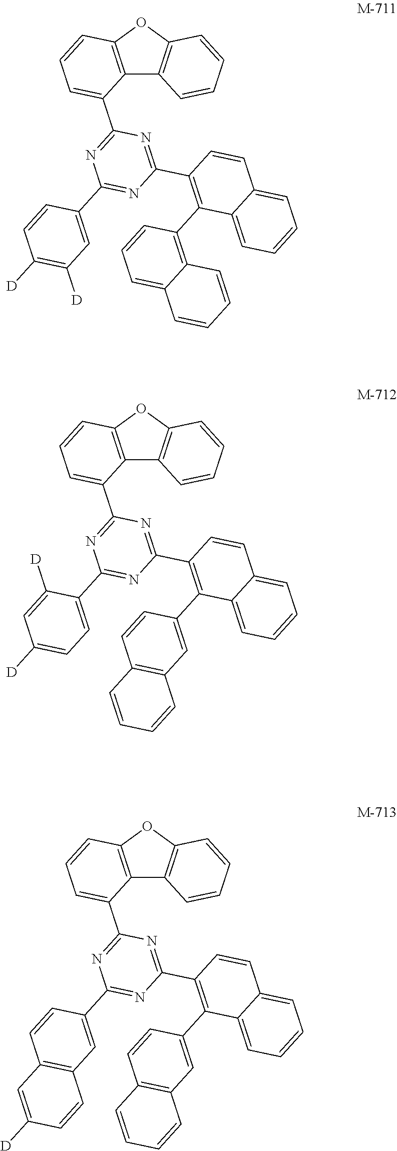 [SMILES: [2H]c1ccc(-c2nc(-c3ccc4ccccc4c3-c3cccc4ccccc34)nc(-c3cccc4oc5ccccc5c34)n2)cc1[2H] ; [2H]c1ccc(-c2nc(-c3ccc4ccccc4c3-c3ccc4ccccc4c3)nc(-c3cccc4oc5ccccc5c34)n2)c([2H])c1 ; [2H]c1ccc2cc(-c3nc(-c4ccc5ccccc5c4-c4ccc5ccccc5c4)nc(-c4cccc5oc6ccccc6c45)n3)ccc2c1]

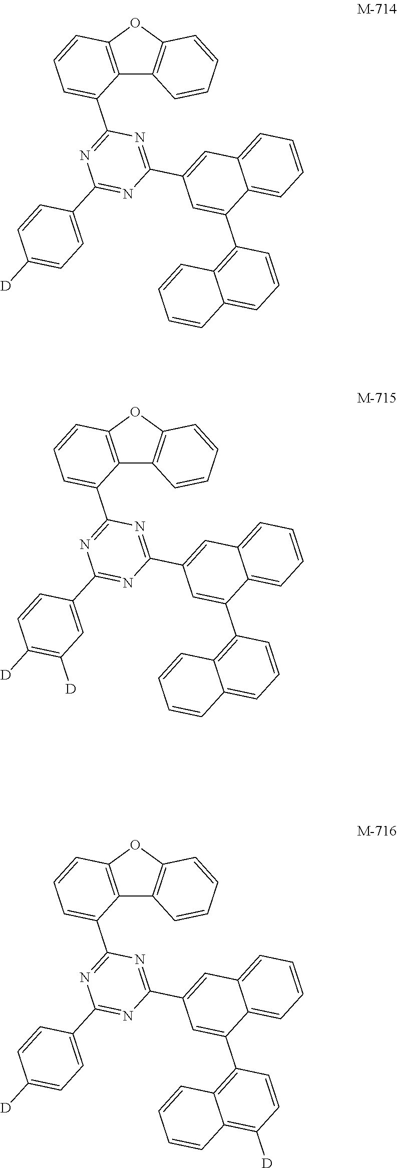 [SMILES: [2H]c1ccc(-c2nc(-c3cc(-c4cccc5ccccc45)c4ccccc4c3)nc(-c3cccc4oc5ccccc5c34)n2)cc1 ; [2H]c1ccc(-c2nc(-c3cc(-c4cccc5ccccc45)c4ccccc4c3)nc(-c3cccc4oc5ccccc5c34)n2)cc1[2H] ; [2H]c1ccc(-c2nc(-c3cc(-c4ccc([2H])c5ccccc45)c4ccccc4c3)nc(-c3cccc4oc5ccccc5c34)n2)cc1]

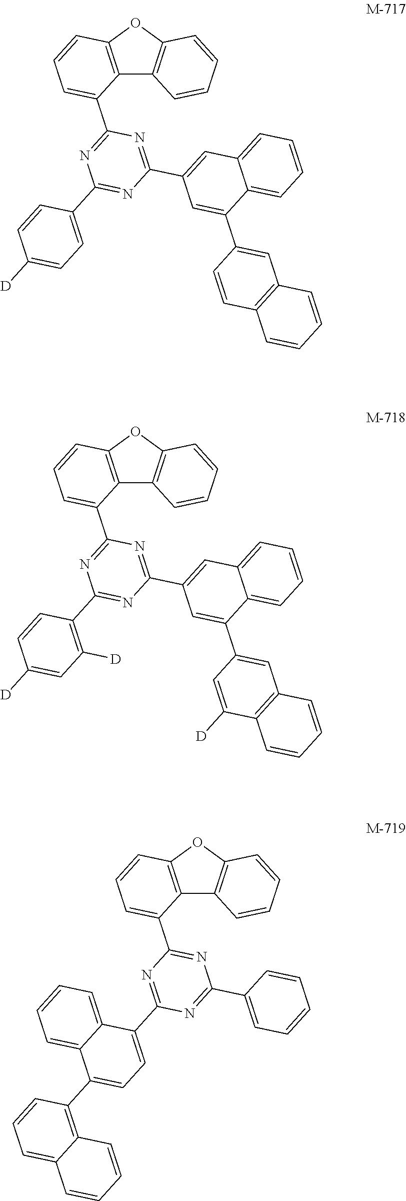 [SMILES: [2H]c1ccc(-c2nc(-c3cc(-c4ccc5ccccc5c4)c4ccccc4c3)nc(-c3cccc4oc5ccccc5c34)n2)cc1 ; [2H]c1ccc(-c2nc(-c3cc(-c4cc([2H])c5ccccc5c4)c4ccccc4c3)nc(-c3cccc4oc5ccccc5c34)n2)c([2H])c1 ; c1ccc(-c2nc(-c3ccc(-c4cccc5ccccc45)c4ccccc34)nc(-c3cccc4oc5ccccc5c34)n2)cc1]

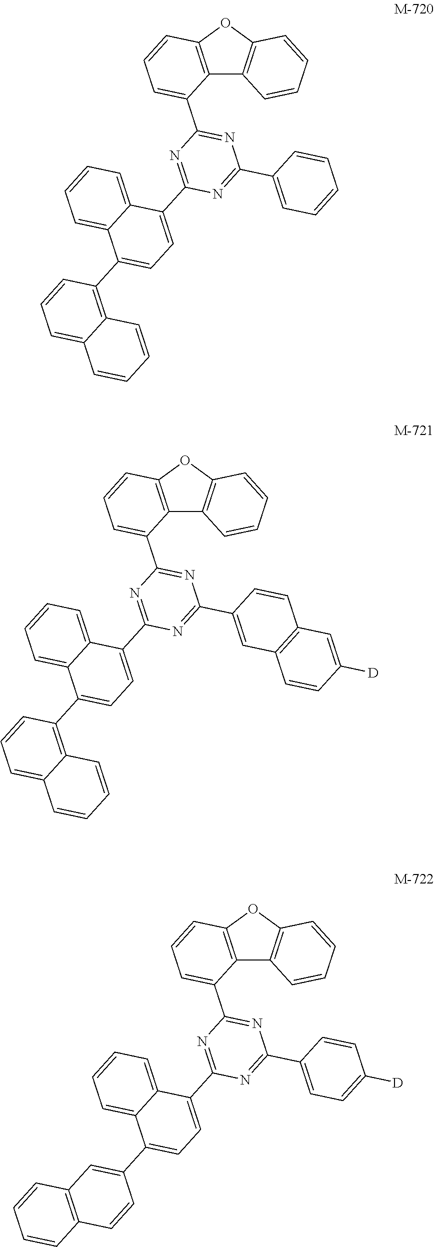 [SMILES: c1ccc(-c2nc(-c3ccc(-c4cccc5ccccc45)c4ccccc34)nc(-c3cccc4oc5ccccc5c34)n2)cc1 ; [2H]c1ccc2cc(-c3nc(-c4ccc(-c5cccc6ccccc56)c5ccccc45)nc(-c4cccc5oc6ccccc6c45)n3)ccc2c1 ; [2H]c1ccc(-c2nc(-c3ccc(-c4ccc5ccccc5c4)c4ccccc34)nc(-c3cccc4oc5ccccc5c34)n2)cc1]

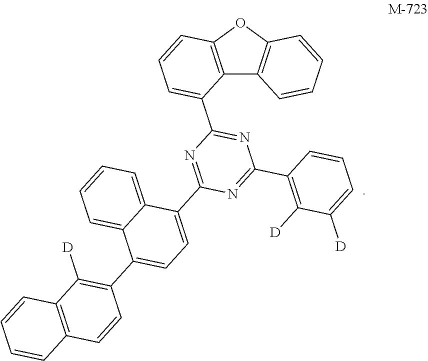 [SMILES: [2H]c1cccc(-c2nc(-c3ccc(-c4ccc5ccccc5c4[2H])c4ccccc34)nc(-c3cccc4oc5ccccc5c34)n2)c1[2H]]

Optionally, in the material composition, a mass ratio of the first compound to the second compound is 1:9-9:1;
optionally, in the material composition, a mass ratio of the first compound to the second compound is 2:8-8:2;
more optionally, in the material composition, a mass ratio of the first compound to the second compound is 3:7-7:3;
further optionally, in the material composition, a mass ratio of the first compound to the second compound is 4:6-6:4.

The present application provides an organic electroluminescent host material composition, comprising the organic material composition described above.

The present application provides the use of the above-mentioned organic material composition or the above-mentioned organic electroluminescent host material composition in an optical device;
optionally, the optical device comprises an organic electroluminescent device.

The present application provides an organic electroluminescent device, wherein the organic electroluminescent device comprises an anode and a cathode, and an organic layer disposed between the anode and the cathode, wherein the organic layer comprises the above-mentioned organic material composition or the above-mentioned organic electroluminescent host material composition described above;
optionally, the organic layer comprises a light-emitting layer, and the light-emitting layer comprises the above-mentioned organic material composition or the above-mentioned organic electroluminescent host material composition.

Optionally, the organic layer comprises a hole injection layer, a hole transport layer, an electron blocking layer, a light emitting layer, a hole blocking layer, an electron transport layer and an electron injection layer stacked in sequence from the anode side to the cathode side.

Optionally, the material of the light-emitting layer comprises a host material and a guest material, and the host material comprises the above-mentioned organic material composition or the above-mentioned organic electroluminescent host material composition.

Optionally, the guest material comprises a phosphorescent dopant, and the phosphorescent dopant comprises a complex containing a transition metal.

The present application provides an organic electroluminescent device, comprising the above-mentioned organic electroluminescent device.

The term “organic electroluminescent material” in the disclosure of the present application means a material that can be used in an organic electroluminescent device and may include at least one compound. If necessary, the organic electroluminescent material may be contained in any layer constituting the organic electroluminescent device. For example, the organic electroluminescent material can be a hole injection material, a hole transport material, a hole-assisting material, an emission-assisting material, an electron blocking material, a luminescent material (containing an organic electroluminescent main material and a dopant material), an electron buffer material, a hole blocking material, an electron transport material, an electron injection material, and so on.

An organic electroluminescent material disclosed in the present application may include one organic electroluminescent material or plurality of organic electroluminescent materials, wherein the plurality of organic electroluminescent materials refer to materials comprising a combination of at least two organic electroluminescent materials, and the materials may be included in any layer constituting an organic electroluminescent device. It may mean both a material included before the organic electroluminescent device (for example, before vapor deposition) and a material included after the organic electroluminescent device (for example, after vapor deposition). For example, the material can be a combination of at least two compositions, which can be included in at least one of the following: a hole injection layer, a hole transport layer, a hole-assisting layer, an emission-assisting layer, an electron blocking layer, a light-emitting layer, an electron buffer layer, a hole blocking layer, an electron transport layer and an electron injection layer. Two compositions of the plurality of organic electroluminescent materials may be contained in the same layer or in different layers, and may be mix-evaporated or co-evaporated, or may be evaporated individually.

The term □organic electroluminescent host material composition□ disclosed in the present application means an organic electroluminescent material including a combination of at least two host materials. It may mean both a material before being included in the organic electroluminescent device (for example, before vapor deposition) and a material after being included in the organic electroluminescent device (for example, after vapor deposition). The composition disclosed in the present application may be included in any light-emitting layer constituting an organic electroluminescent device. Two or more compounds among the plurality of host materials included in the composition disclosed in the present application may be included in one light-emitting layer, or may be respectively included in different light-emitting layers. For example, when two or more host materials are included in one layer, the layer may be formed by mixed evaporation, or may be simultaneously formed by separate co-evaporations.

In the present application, the first compound can be prepared by the following synthetic route, comprising the following steps:
1. Synthesis of Intermediate Nn-A: the reaction raw materials Nn-1 and Nn-2 are subjected to Suzuki cross-coupling reaction, and the reaction equation is as follows:

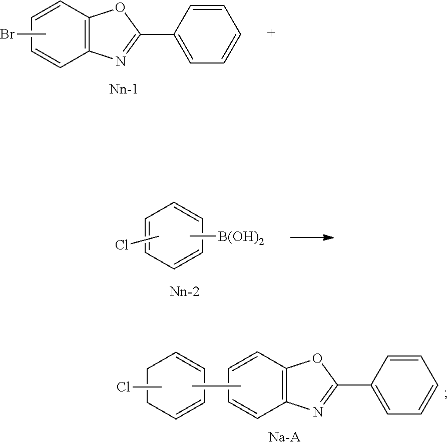

2. Synthesis of compound N-n: intermediate Nn-A and Nn-B undergo Buchwald-Hartwig cross-coupling reaction, the reaction equation is as follows:

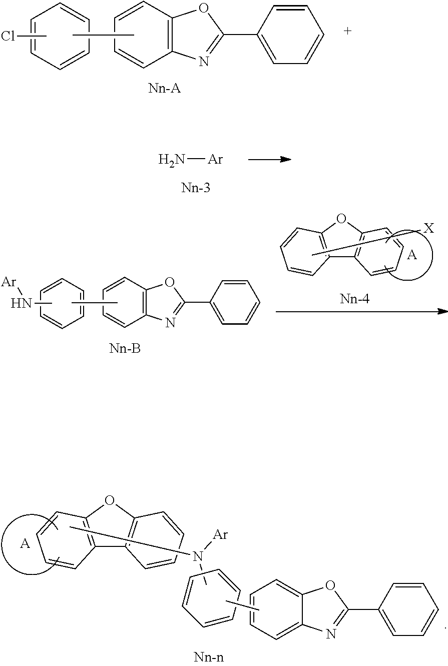

The beneficial effects of the present application are as follows.

The organic material composition of the present application comprises a first compound and a second compound, wherein the first compound has a structure as shown in Formula (1), and the second compound has a structure as shown in Formula (2); the first compound having a structure as shown in Formula (1) and the second compound having a structure as shown in Formula (2) cooperate with each other, which is beneficial for matching HOMO and LUMO energy levels with adjacent energy levels, so that the organic material composition obtains higher stability and more balanced carrier mobility, thereby making the organic electroluminescent device containing the material have a better lifespan, and also has a lower driving voltage and higher efficiency.

### BRIEF DESCRIPTION OF THE DRAWINGS

In order to more clearly illustrate the specific implementation methods of the present application or the technical solutions in the prior art, the drawings required for use in the specific implementation methods or the description of the prior art will be briefly introduced below.

Obviously, the drawings described below are some implementation methods of the present application. For those skilled in the art, other drawings can be obtained based on these drawings without paying creative work.

FIG. 1 is a structural diagram of an organic electroluminescent device in an embodiment of the device according to the present application;

wherein the reference signs are as follows: 1—substrate; 2—anode; 3—hole injection layer; 4—hole transport layer; 5—light-emitting layer; 6—electron transport layer; 7—electron injection layer; 8—cathode.

### DETAILED DESCRIPTION

The following examples are provided for a better understanding of the present application, but are not intended to limit the best implementation, nor to limit the content and protection scope of the present application. Any product identical or similar to the present application obtained by anyone under the inspiration of the present application or by combining the features of the present application with other prior arts shall fall within the protection scope of the present application.

If no specific experimental steps or conditions are specified in the examples, the experiments can be carried out according to the conventional experimental steps or conditions described in the literature in the art. The reagents or instruments used without indicating the manufacturer are all conventional reagent products that can be obtained through commercial purchase.

### Example 1

This example provides an oxazole organic compound N-1 containing benzonaphthofuran. The synthesis of the oxazole organic compound N-1 containing benzonaphthofuran specifically comprises the following steps:

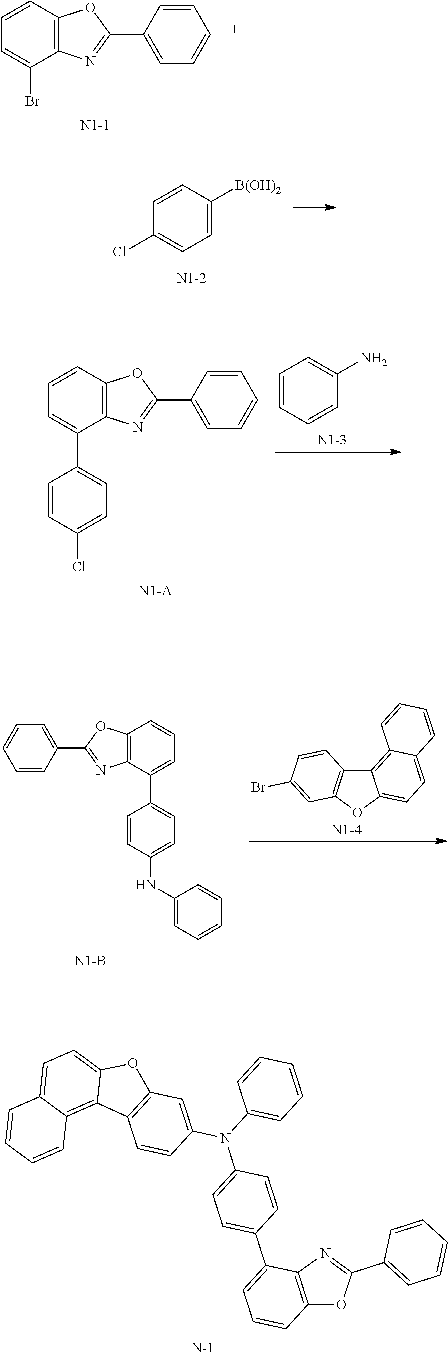

### Synthesis of Intermediate N1-A

After replacing the air in a three port reaction bottle equipped with mechanical stirring, thermometer, and condenser tube with nitrogen, the raw materials 10 g of N1-1 (36.63 mmol), 5.72 g of N1-2 (36.63 mmol), 0.85 g of tetrakistriphenylphosphine palladium (0.73 mmol), 10.11 g of potassium carbonate (73.27 mmol), 70 mL of toluene, 30 mL of ethanol and 30 mL of water were added in sequence. The mixture was stirred at 65° C. for 2 hours. After the reaction was completed, the organic layer was extracted with ethyl acetate (EA), and residual moisture was removed by using anhydrous sodium sulfate, and the residue was dried and purified by column chromatography to obtain 10.06 g of compound N1-A (yield 90%).

### Synthesis of Intermediate N1-B

After replacing the air in a three port reaction bottle equipped with mechanical stirring, thermometer, and condenser tube with nitrogen, 10 g of the intermediate N1-A (32.78 mmol), 3.1 g of N1-3 (32.78 mmol), 0.6 g of tris(dibenzylideneacetone)dipalladium (0.66 mmol), 0.67 g of 2-dicyclohexylphosphine-2′,6′-dimethoxybiphenyl (1.64 mmol), 6.3 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 8.31 g of compound N1-B (yield 82%).

### Synthesis of Compound N-1

After replacing the air in a three port reaction bottle equipped with mechanical stirring, thermometer, and condenser tube with nitrogen, 8.30 g of the intermediate N1-B (26.85 mmol), 7.95 g of N1-4 (26.85 mmol), 0.49 g of tris(dibenzylideneacetone)dipalladium (0.54 mmol), 0.55 g of 2-dicyclohexylphosphine-2′,6′-dimethoxybiphenyl (1.34 mmol), 5.16 g of sodium tert-butoxide (53.70 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 11.80 g of benzonaphthofuran-containing oxazole organic compound N-1 (yield 76%).

Elemental analysis: C₄₁H₂₆N₂O₂; theoretical value: C, 85.10; H, 4.53; N, 4.84; O, 5.53; measured value: C, 85.12; H, 4.52; N, 4.83; HRMS(ESI) m/z [M+H]+: theoretical value: 578.20; measured value: 579.21.

### Example 2

This example provides an oxazole organic compound N-6 containing benzonaphthofuran. The synthesis of the oxazole organic compound N-6 containing benzonaphthofuran specifically comprises the following steps:

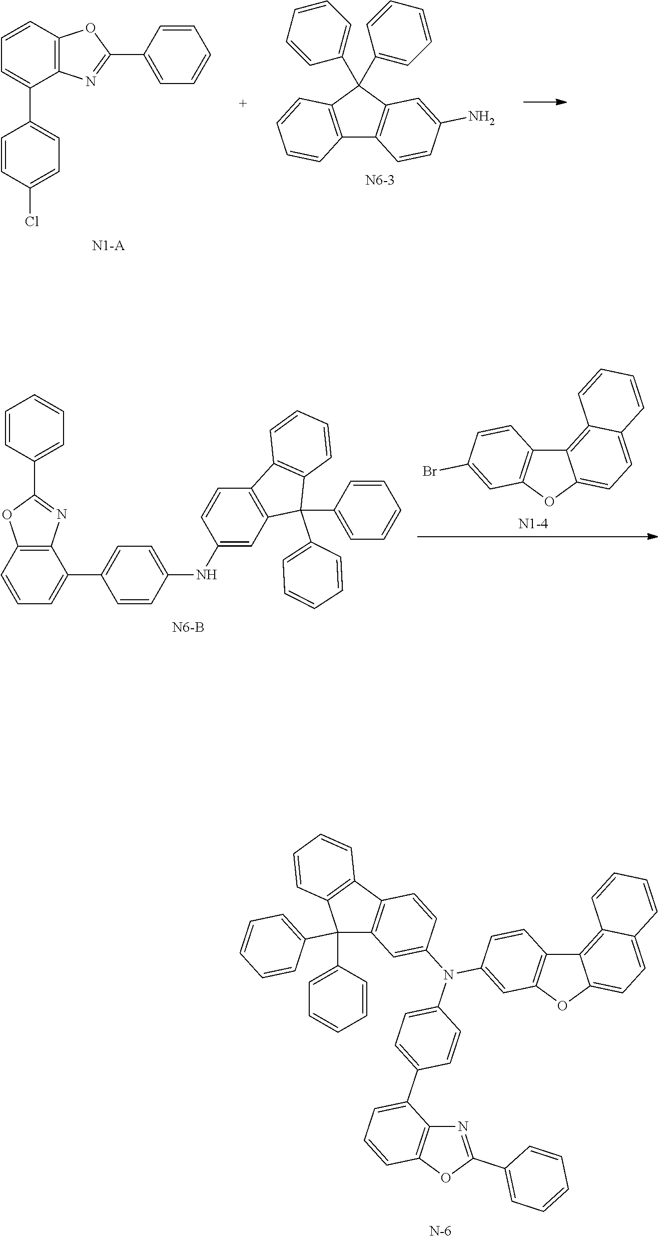

After replacing the air in a three port reaction bottle equipped with mechanical stirring, thermometer, and condenser tube with nitrogen, 10 g of the intermediate N1-A (32.78 mmol), 10.92 g of N6-3 (32.78 mmol), 0.6 g of tris(dibenzylideneacetone)dipalladium (0.66 mmol), 0.67 g of 2-dicyclohexylphosphine-2′,6′-dimethoxybiphenyl (1.64 mmol), 6.3 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 14.41 g of compound N6-B (yield 73%).

After replacing the air in a three port reaction bottle equipped with mechanical stirring, thermometer, and condenser tube with nitrogen, 10 g of the intermediate N6-B (16.60 mmol), 4.91 g of N1-4 (16.60 mmol), 0.30 g of tris(dibenzylideneacetone)dipalladium (0.33 mmol), 0.34 g of 2-dicyclohexylphosphine-2′,6′-dimethoxybiphenyl (0.83 mmol), 3.19 g of sodium tert-butoxide (33.21 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 9.24 g of benzonaphthofuran-containing oxazole organic compound N-6 (yield 68%).

Elemental analysis: C₆₀H₃₈N₂O₂; theoretical value: C, 88.00; H, 4.68; N, 3.42; O, 3.91; measured value: C, 88.01; H, 4.68; N, 3.41; HRMS(ESI) m/z [M+H]+: theoretical value: 818.29; measured value: 819.28.

### Example 3

This example provides an oxazole organic compound N-18 containing benzonaphthofuran. The synthesis of the oxazole organic compound N-18 containing benzonaphthofuran specifically comprises the following steps:

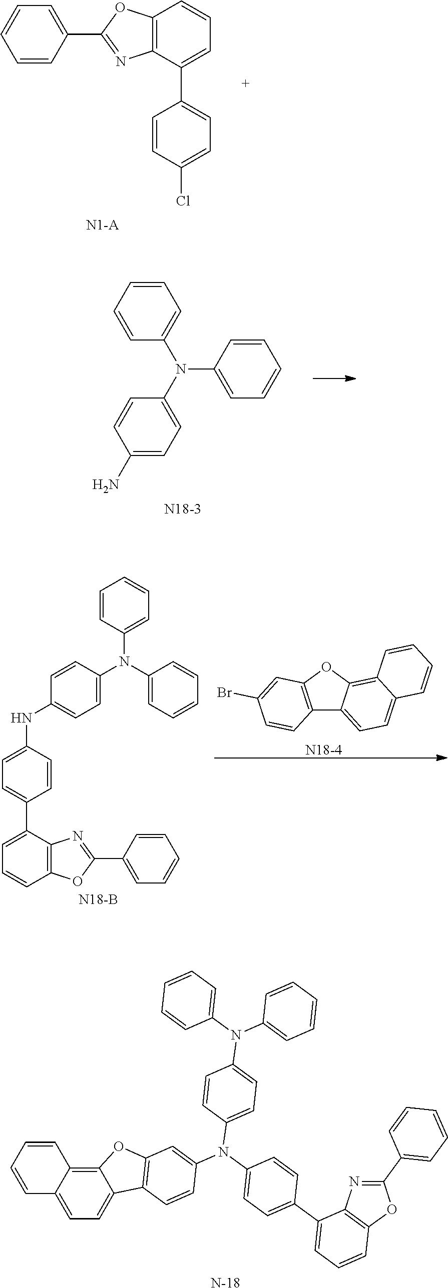

After replacing the air in a three port reaction bottle equipped with mechanical stirring, thermometer, and condenser tube with nitrogen, 10 g of the intermediate N1-A (32.78 mmol), 8.53 g of N18-3 (32.78 mmol), 0.60 g of tris(dibenzylideneacetone)dipalladium (0.66 mmol), 0.67 g of 2-dicyclohexylphosphine-2′,6′-dimethoxybiphenyl (1.64 mmol), 6.3 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 13.19 g of compound N18-B (yield 76%).

After replacing the air in a three port reaction bottle equipped with mechanical stirring, thermometer, and condenser tube with nitrogen, 10 g of N18-B (18.90 mmol), 5.59 g of N18-4 (18.90 mmol), 0.35 g of tris(dibenzylideneacetone)dipalladium (0.38 mmol), 0.39 g of 2-dicyclohexylphosphine-2′,6′-dimethoxybiphenyl (0.94 mmol), 3.63 g of sodium tert-butoxide (37.79 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.70 g of organic electroluminescent compound N-18 (yield 76%).

Elemental analysis: C₅₃H₃₅N₃O₂; theoretical value: C, 85.35; H, 4.73; N, 5.63; O, 4.29; measured value: C, 85.37; H, 4.72; N, 5.62; HRMS(ESI) m/z [M+H]+: theoretical value: 745.27; measured value: 746.26.

### Example 4

This example provides an oxazole organic compound N-30 containing benzonaphthofuran. The synthesis of the oxazole organic compound N-30 containing benzonaphthofuran specifically comprises the following steps:

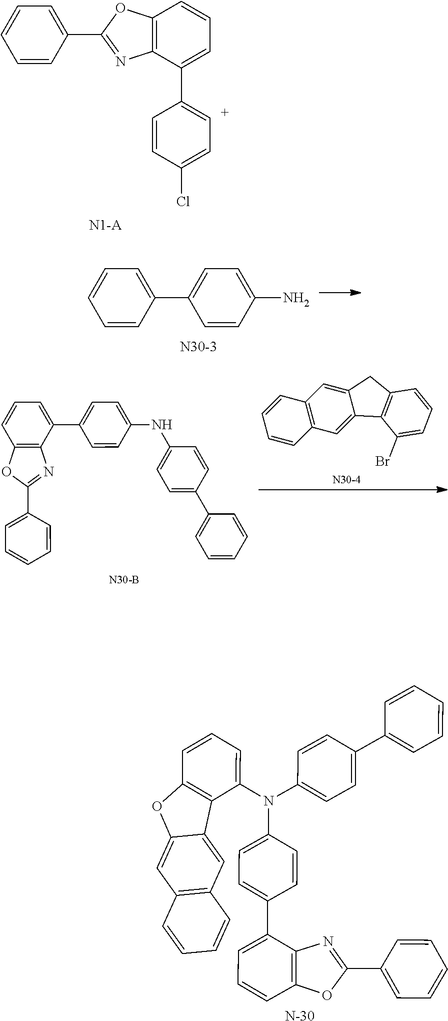

After replacing the air in a three port reaction bottle equipped with mechanical stirring, thermometer, and condenser tube with nitrogen, 10 g of the intermediate N1-A (32.78 mmol), 5.54 g of N30-3 (32.78 mmol), 0.60 g of tris(dibenzylideneacetone)dipalladium (0.66 mmol), 0.67 g of 2-dicyclohexylphosphine-2′,6′-dimethoxybiphenyl (1.64 mmol), 6.30 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 11.49 g of compound N30-B (yield 80%).

After replacing the air in a three port reaction bottle equipped with mechanical stirring, thermometer, and condenser tube with nitrogen, 10 g of the intermediate N30-B (22.82 mmol), 6.75 g of N30-4 (22.82 mmol), 0.42 g of tris(dibenzylideneacetone)dipalladium (0.46 mmol), 0.47 g of 2-dicyclohexylphosphine-2′,6′-dimethoxybiphenyl (1.14 mmol), 4.39 g of sodium tert-butoxide (45.64 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.75 g of benzonaphthofuran-containing oxazole organic compound N-30 (yield 72%).

Elemental analysis: C₄₇H₃₀N₂O₂; theoretical value: C, 86.22; H, 4.62; N, 4.28; O, 4.89; measured value: C, 86.20; H, 4.63; N, 4.29; HRMS(ESI) m/z [M+H]+: theoretical value: 654.23; measured value: 654.24.

### Example 5

This example provides an oxazole organic compound N-45 containing benzonaphthofuran. The synthesis of the oxazole organic compound N-45 containing benzonaphthofuran specifically comprises the following steps:

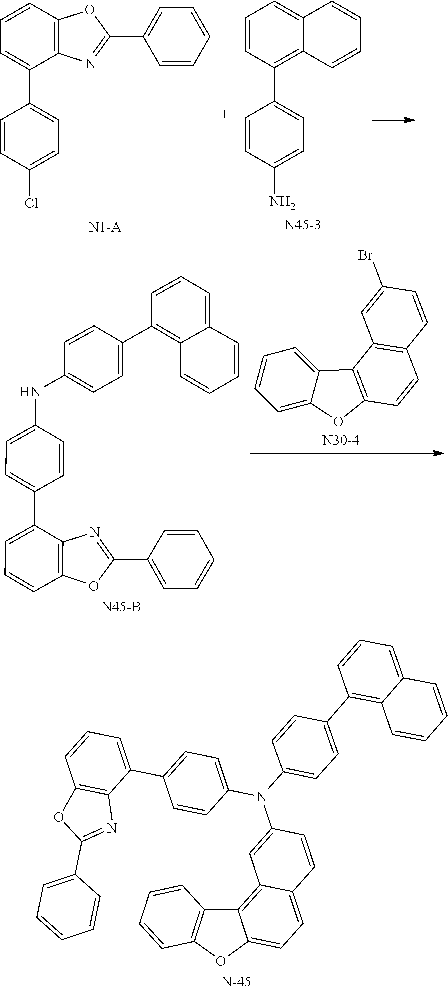

After replacing the air in a three port reaction bottle equipped with mechanical stirring, thermometer, and condenser tube with nitrogen, 10 g of the intermediate N1-A (32.78 mmol), 7.18 g of N45-3 (32.78 mmol), 0.60 g of tris(dibenzylideneacetone)dipalladium (0.66 mmol), 0.67 g of 2-dicyclohexylphosphine-2′,6′-dimethoxybiphenyl (1.64 mmol), 6.30 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 12.16 g of compound N45-B (yield 76%).

After replacing the air in a three port reaction bottle equipped with mechanical stirring, thermometer, and condenser tube with nitrogen, 7.50 g of the intermediate N45-B (15.36 mmol), 3.87 g of N45-4 (15.36 mmol), 0.28 g of tris(dibenzylideneacetone)dipalladium (0.31 mmol), 0.31 g of 2-dicyclohexylphosphine-2′,6′-dimethoxybiphenyl (0.77 mmol), 2.95 g of sodium tert-butoxide (30.73 mmol) and 80 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 8.22 g of benzonaphthofuran-containing oxazole organic compound N-45 (yield 76%).

Elemental analysis: C₅₁H₃₂N₂O₂; theoretical value: C, 86.91; H, 4.58; N, 3.97; O, 4.54; measured value: C, 86.93; H, 4.57; N, 3.96; HRMS(ESI) m/z [M+H]+: theoretical value: 704.25; measured value: 705.26.

### Example 6

This example provides an oxazole organic compound N-72 containing benzonaphthofuran. The synthesis of the oxazole organic compound N-72 containing benzonaphthofuran specifically comprises the following steps:

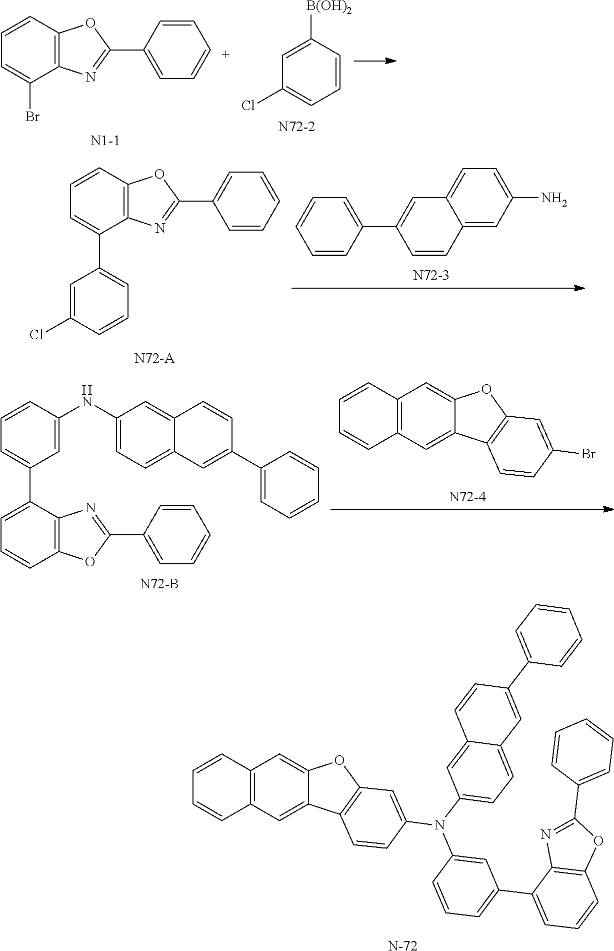

### Synthesis of Intermediate N72-A

After replacing the air in a three port reaction bottle equipped with mechanical stirring, thermometer, and condenser tube with nitrogen, the raw materials 10 g of N1-1 (36.63 mmol), 5.72 g of N72-2 (36.63 mmol), 0.85 g of tetrakistriphenylphosphine palladium (0.73 mmol), 10.11 g of potassium carbonate (73.27 mmol), 70 mL of toluene, 30 mL of ethanol and 30 mL of water were added in sequence. The mixture was stirred at 65° C. for 2 hours. After the reaction was completed, the organic layer was extracted with ethyl acetate (EA), and residual moisture was removed by using anhydrous sodium sulfate, the residue was dried and purified by column chromatography to obtain 9.50 g of compound N72-A (yield 85%).

### Synthesis of Intermediate N72-B

After replacing the air in a three port reaction bottle equipped with mechanical stirring, thermometer, and condenser tube with nitrogen, 9.50 g of the intermediate N72-A (31.14 mmol), 6.82 g of N73-3 (31.14 mmol), 0.57 g of tris(dibenzylideneacetone)dipalladium (0.62 mmol), 0.64 g of 2-dicyclohexylphosphine-2′,6′-dimethoxybiphenyl (1.56 mmol), 5.99 g of sodium tert-butoxide (62.28 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 11.10 g of compound N72-B (yield 73%).

After replacing the air in a three port reaction bottle equipped with mechanical stirring, thermometer, and condenser tube with nitrogen, 10 g of the intermediate N72-B (20.48 mmol), 6.06 g of N72-4 (20.48 mmol), 0.37 g of tris(dibenzylideneacetone)dipalladium (0.41 mmol), 0.42 g of 2-dicyclohexylphosphine-2′,6′-dimethoxybiphenyl (1.02 mmol), 3.94 g of sodium tert-butoxide (40.97 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.82 g of benzonaphthofuran-containing oxazole organic compound N-72 (yield 75%).

Elemental analysis: C₅₁H₃₂N₂O₂; theoretical value: C, 86.91; H, 4.58; N, 3.97; O, 4.54; measured value: C, 86.93; H, 4.57; N, 3.96; HRMS(ESI) m/z [M+H]+: theoretical value: 704.25; measured value: 705.26.

### Example 7

This example provides an oxazole organic compound N-95 containing benzonaphthofuran. The synthesis of the oxazole organic compound N-95 containing benzonaphthofuran specifically comprises the following steps:

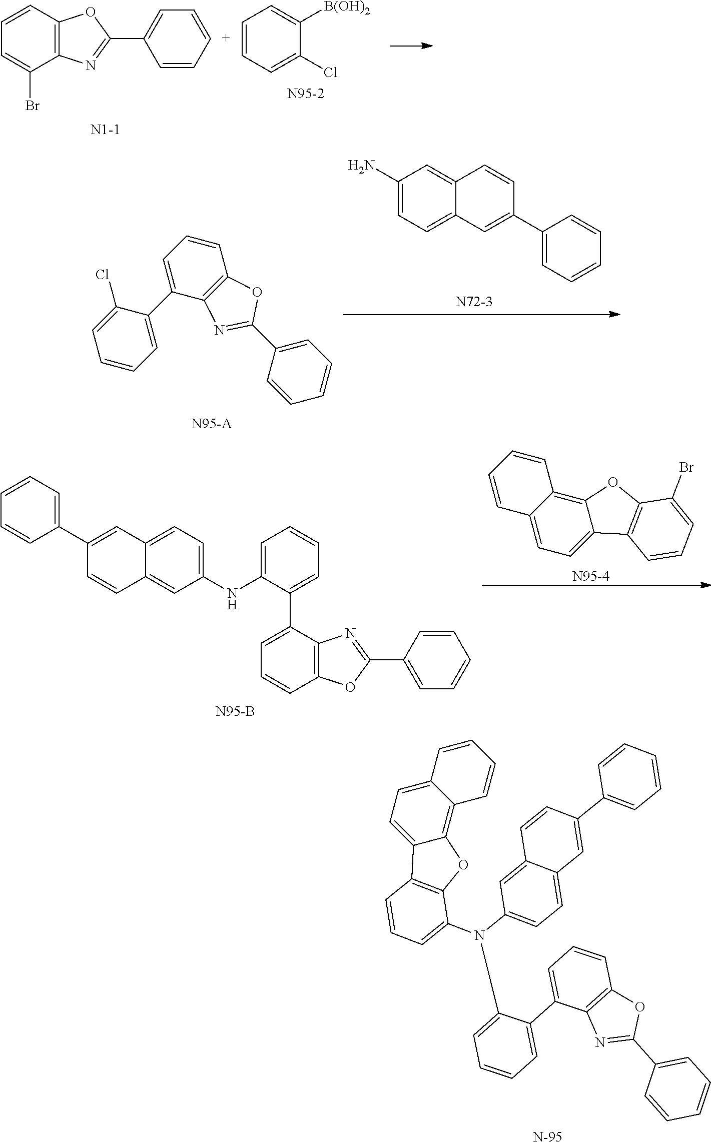

After replacing the air in a three port reaction bottle equipped with mechanical stirring, thermometer, and condenser tube with nitrogen, the raw materials 10 g of N1-1 (36.63 mmol), 5.72 g of N95-2 (36.63 mmol), 0.85 g of tetrakistriphenylphosphine palladium (0.73 mmol), 10.11 g of potassium carbonate (73.27 mmol), 70 mL of toluene, 30 mL of ethanol and 30 mL of water were added in sequence. The mixture was stirred at 65° C. for 2 hours. After the reaction was completed, the organic layer was extracted with ethyl acetate (EA), and residual moisture was removed by using anhydrous sodium sulfate, and the residue was dried and purified by column chromatography to obtain 9.28 g of compound N95-A (yield 83%).

After replacing the air in a three port reaction bottle equipped with mechanical stirring, thermometer, and condenser tube with nitrogen, 9.28 g of the intermediate N95-A (30.42 mmol), 6.67 g of N72-3 (30.42 mmol), 0.56 g of tris(dibenzylideneacetone)dipalladium (0.61 mmol), 0.62 g of 2-dicyclohexylphosphine-2′,6′-dimethoxybiphenyl (1.52 mmol), 5.85 g of sodium tert-butoxide (60.84 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 10.69 g of compound N95-B (yield 72%).

After replacing the air in a three port reaction bottle equipped with mechanical stirring, thermometer, and condenser tube with nitrogen, 10 g of the intermediate N95-B (20.48 mmol), 6.06 g of N95-4 (20.48 mmol), 0.37 g of tris(dibenzylideneacetone)dipalladium (0.41 mmol), 0.42 g of 2-dicyclohexylphosphine-2′,6′-dimethoxybiphenyl (1.02 mmol), 3.94 g of sodium tert-butoxide (40.97 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.10 g of benzonaphthofuran-containing oxazole organic compound N-95 (yield 70%).

Elemental analysis: C₅₁H₃₂N₂O₂; theoretical value: C, 86.91; H, 4.58; N, 3.97; O, 4.54; measured value: C, 86.90; H, 4.58; N, 3.98; HRMS(ESI) m/z [M+H]+: theoretical value: 704.25; measured value: 705.26.

### Example 8

This example provides an oxazole organic compound N-134 containing benzonaphthofuran. The synthesis of the oxazole organic compound N-134 containing benzonaphthofuran specifically comprises the following steps:

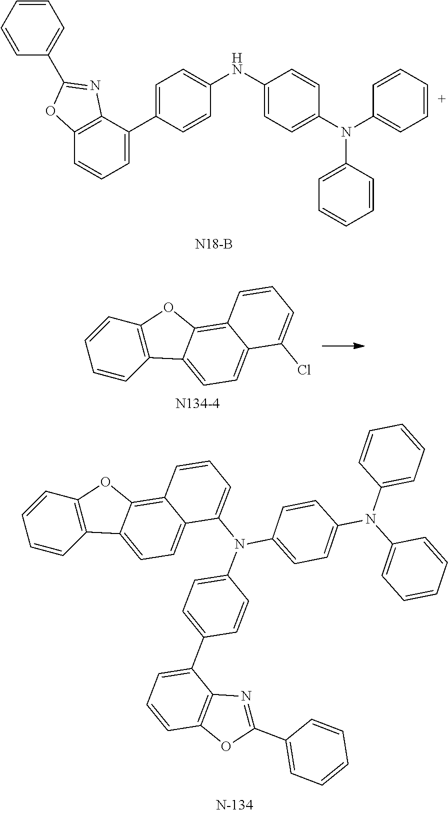 [SMILES: c1ccc(-c2nc3c(-c4ccc(Nc5ccc(N(c6ccccc6)c6ccccc6)cc5)cc4)cccc3o2)cc1 ; Clc1cccc2c1ccc1c3ccccc3oc21 ; "N134-4" ; c1ccc(-c2nc3c(-c4ccc(N(c5ccc(N(c6ccccc6)c6ccccc6)cc5)c5cccc6c5ccc5c7ccccc7oc65)cc4)cccc3o2)cc1]

After replacing the air in a three port reaction bottle equipped with mechanical stirring, thermometer, and condenser tube with nitrogen, 8.60 g of the intermediate N18-B (16.25 mmol), 4.10 g of N134-4 (16.25 mmol), 0.30 g of tris(dibenzylideneacetone)dipalladium (0.33 mmol), 0.33 g of 2-dicyclohexylphosphine-2′,6′-dimethoxybiphenyl (0.81 mmol), 3.12 g of sodium tert-butoxide (32.50 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 9.20 g of benzonaphthofuran-containing oxazole organic compound N-134 (yield 76%).

Elemental analysis: C₅₃H₃₅N₃O₂; theoretical value: C, 85.35; H, 4.73; N, 5.63; O, 4.29; measured value: C, 85.33; H, 4.74; N, 5.64; HRMS(ESI) m/z [M+H]+: theoretical value: 745.27; measured value: 746.29.

### Example 9

This example provides an oxazole organic compound N-148 containing benzonaphthofuran. The synthesis of the oxazole organic compound N-148 containing benzonaphthofuran specifically comprises the following steps:

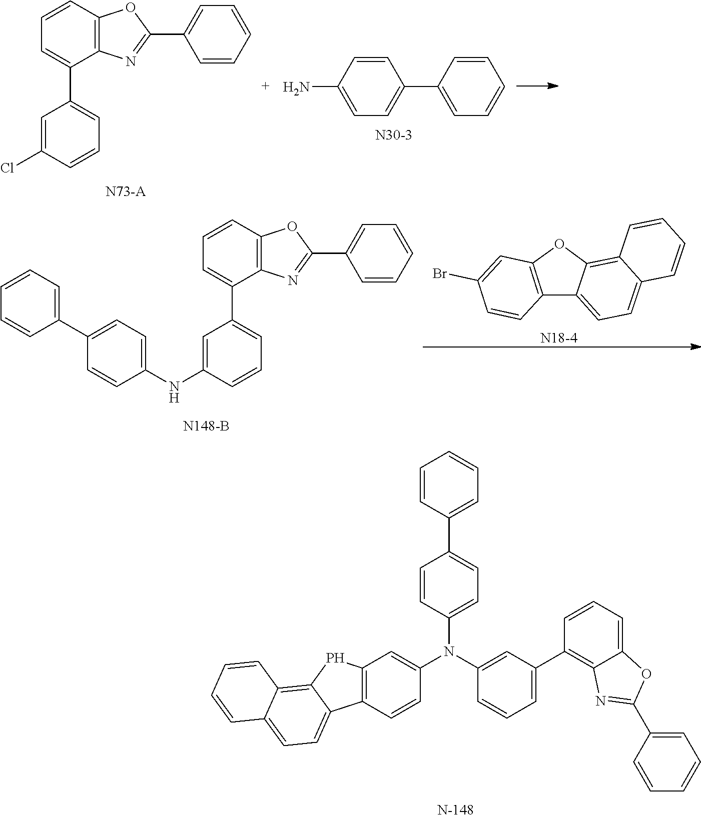

After replacing the air in a three port reaction bottle equipped with mechanical stirring, thermometer, and condenser tube with nitrogen, the intermediate 10 g of N73-A (32.78 mmol), 5.54 g of N30-3 (32.78 mmol), 0.60 g of tris(dibenzylideneacetone)dipalladium (0.66 mmol), 0.67 g of 2-dicyclohexylphosphine-2′,6′-dimethoxybiphenyl (1.64 mmol), 6.3 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 11.34 g of compound N148-B (yield 79%).

After replacing the air in a three port reaction bottle equipped with mechanical stirring, thermometer, and condenser tube with nitrogen, 10 g of the intermediate N148-B (22.82 mmol), 6.57 g of N18-4 (22.82 mmol), 0.42 g of tris(dibenzylideneacetone)dipalladium (0.46 mmol), 0.47 g of 2-dicyclohexylphosphine-2′,6′-dimethoxybiphenyl (1.14 mmol), 4.39 g of sodium tert-butoxide (45.64 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 11.20 g of benzonaphthofuran-containing oxazole organic compound N-148 (yield 75%).

Elemental analysis: C₄₇H₃₀N₂O₂; theoretical value: C, 86.22; H, 4.62; N, 4.28; O, 4.89; measured value: C, 86.22; H, 4.62; N, 4.28; HRMS(ESI) m/z [M+H]+: theoretical value: 654.23; measured value: 655.21.

### Example 10

This example provides an organic electroluminescent compound N-173. The synthesis of the organic electroluminescent compound N-173 specifically comprises the following steps:

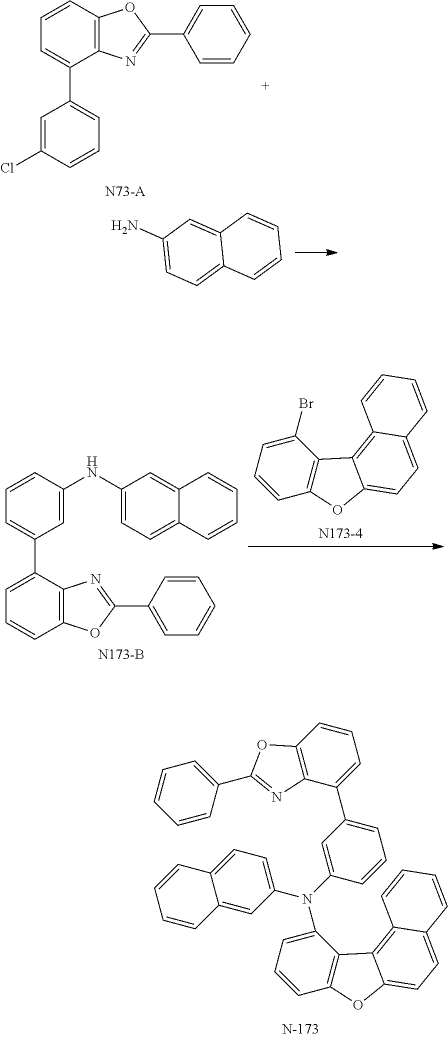

After replacing the air in a three port reaction bottle equipped with mechanical stirring, thermometer, and condenser tube with nitrogen, the intermediate 10 g of N73-A (32.78 mmol), 4.69 g of N173-3 (32.78 mmol), 0.60 g of tris(dibenzylideneacetone)dipalladium (0.66 mmol), 0.67 g of 2-dicyclohexylphosphine-2′,6′-dimethoxybiphenyl (1.64 mmol), 6.30 g of sodium tert-butoxide (65.56 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, and the residue was dried and purified by column chromatography to obtain 9.73 g of compound N173-B (yield 72%).

After replacing the air in a three port reaction bottle equipped with mechanical stirring, thermometer, and condenser tube with nitrogen, 9.70 g of the intermediate N173-B (23.53 mmol), 6.97 g of N173-4 (23.53 mmol), 0.43 g of tris(dibenzylideneacetone)dipalladium (0.47 mmol), 0.48 g of 2-dicyclohexylphosphine-2′,6′-dimethoxybiphenyl (1.18 mmol), 4.52 g of sodium tert-butoxide (47.07 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.35 g of compound N-173 (yield 70%).

Elemental analysis: C₄₅H₂₈N₂O₂; theoretical value: C, 85.97; H, 4.49; N, 4.46; O, 5.09; measured value: C, 85.96; H, 4.48; N, 4.48; HRMS(ESI) m/z [M+H]+: theoretical value: 628.22; measured value: 629.24.

### Example 11

This example provides an organic electroluminescent compound N-182. The synthesis of the organic electroluminescent compound N-182 specifically comprises the following steps:

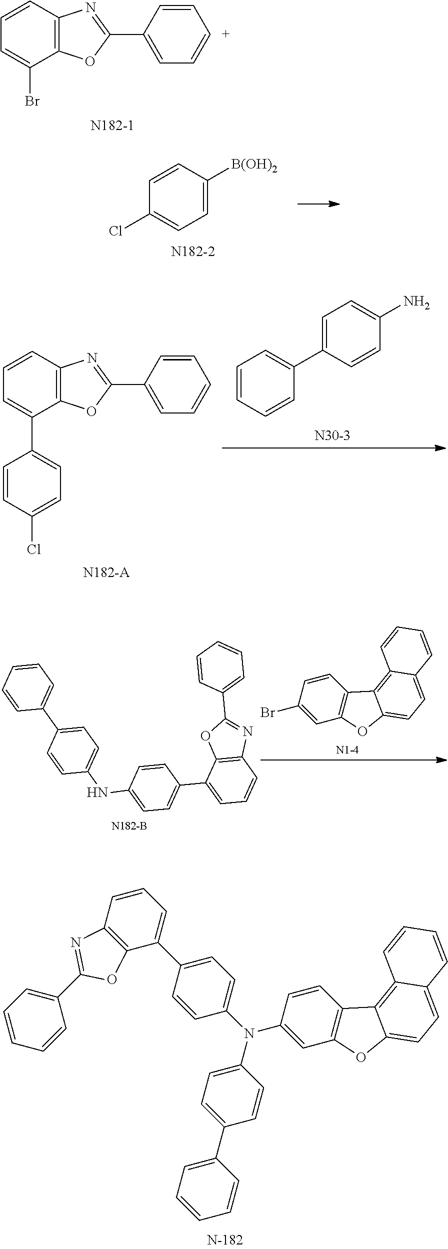

After replacing the air in a three port reaction bottle equipped with mechanical stirring, thermometer, and condenser tube with nitrogen, the raw materials 10 g of N182-1 (36.63 mmol), 5.72 g of N182-2 (36.63 mmol), 0.85 g of tetrakistriphenylphosphine palladium (0.73 mmol), 10.11 g of potassium carbonate (73.27 mmol), 70 mL of toluene, 30 mL of ethanol and 30 mL of water were added in sequence. The mixture was stirred at 65° C. for 2 hours. After the reaction was completed, the organic layer was extracted with ethyl acetate (EA), and residual moisture was removed by using anhydrous sodium sulfate, and the residue was dried and purified by column chromatography to obtain 8.94 g of compound N182-A (yield 80%).

After replacing the air in a three port reaction bottle equipped with mechanical stirring, thermometer, and condenser tube with nitrogen, 8.94 g of the intermediate N182-A (29.31 mmol), 4.96 g of N30-3 (29.31 mmol), 0.54 g of tris(dibenzylideneacetone) dipalladium (0.59 mmol), 0.60 g of 2-dicyclohexylphosphine-2′,6′-dimethoxybiphenyl (1.47 mmol), 5.63 g of sodium tert-butoxide (58.61 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.01 g of compound N182-B (yield 78%).

After replacing the air in a three port reaction bottle equipped with mechanical stirring, thermometer, and condenser tube with nitrogen, 10.01 g of the intermediate N182-B (22.85 mmol), 6.76 g of N1-4 (22.85 mmol), 0.42 g of tris(dibenzylideneacetone) dipalladium (0.46 mmol), 0.47 g of 2-dicyclohexylphosphine-2′,6′-dimethoxybiphenyl (1.14 mmol), 4.39 g of sodium tert-butoxide (45.69 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 11.21 g of compound N-182 (yield 75%).

Elemental analysis: C₄₇H₃₀N₂O₂; theoretical value: C, 86.22; H, 4.62; N, 4.28; O, 4.89; measured value: C, 86.24; H, 4.61; N, 4.27; HRMS(ESI) m/z [M+H]+: theoretical value: 654.23; measured value: 654.21.

### Example 12

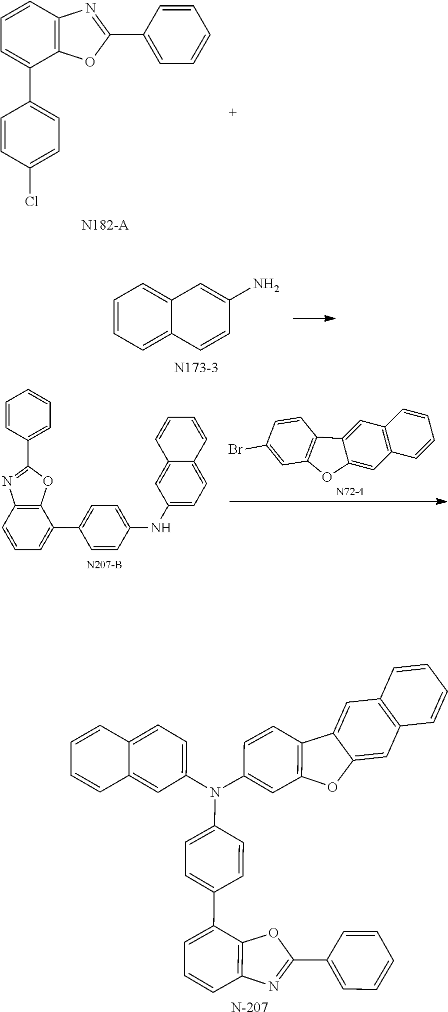

After replacing the air in a three port reaction bottle equipped with mechanical stirring, thermometer, and condenser tube with nitrogen, 8.94 g of the intermediate N182-A (29.31 mmol), 4.96 g of N173-3 (29.31 mmol), 0.54 g of tris(dibenzylideneacetone) dipalladium (0.59 mmol), 0.60 g of 2-dicyclohexylphosphine-2′,6′-dimethoxybiphenyl (1.47 mmol), 5.63 g of sodium tert-butoxide (58.61 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.01 g of compound N207-B (yield 78%).

After replacing the air in a three port reaction bottle equipped with mechanical stirring, thermometer, and condenser tube with nitrogen, 10 g of the intermediate N207-B (24.26 mmol), 7.18 g of N72-4 (24.26 mmol), 0.44 g of tris(dibenzylideneacetone)dipalladium (0.49 mmol), 0.50 g of 2-dicyclohexylphosphine-2′,6′-dimethoxybiphenyl (1.21 mmol), 4.66 g of sodium tert-butoxide (48.52 mmol) and 100 mL of toluene were added in sequence, and the mixture was refluxed and stirred at 110° C. for 3 hours. After the reaction was completed, the organic layer was extracted with ethyl acetate (EA), and residual moisture was removed by using anhydrous sodium sulfate, the residue was dried and purified by column chromatography to obtain 10.97 g of compound N-207 (yield 72%).

Elemental analysis: C₄₅H₂₈N₂O₂; theoretical value: C, 85.97; H, 4.49; N, 4.46; O, 5.09; measured value: C, 85.95; H, 4.49; N, 4.48; HRMS(ESI) m/z [M+H]+: theoretical value: 628.22; measured value: 628.21.

The preparation methods of Examples 13-21 are similar to those of Example 1. Specifically, the raw materials used in Examples 13-21 and the products obtained are shown in Table 1 below.

TABLE 1

| Example | Intermediate Nn-B | Raw material Nn-4 | Product | Yield % |
|---|---|---|---|---|
| 13 | 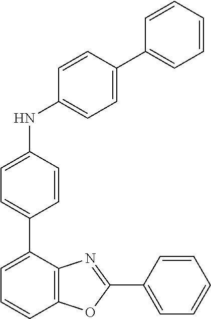 [SMILES: c1ccc(-c2ccc(Nc3ccc(-c4cccc5oc(-c6ccccc6)nc45)cc3)cc2)cc1] | 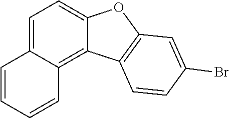 [SMILES: Brc1ccc2c(c1)oc1ccc3ccccc3c12] | 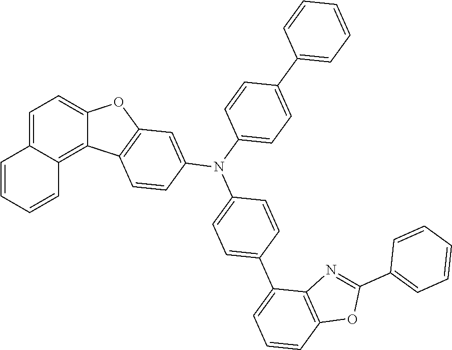 [SMILES: c1ccc(-c2ccc(N(c3ccc(-c4cccc5oc(-c6ccccc6)nc45)cc3)c3ccc4c(c3)oc3ccc5ccccc5c34)cc2)cc1] | 78% |
| | N30-B | N1-4 | N-3 | |
| 14 | 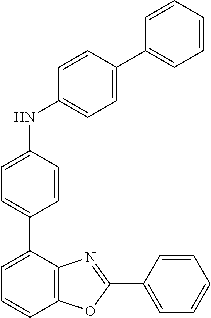 [SMILES: c1ccc(-c2ccc(Nc3ccc(-c4cccc5oc(-c6ccccc6)nc45)cc3)cc2)cc1] | 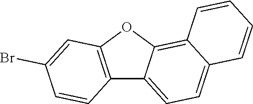 [SMILES: Brc1ccc2c(c1)oc1c3ccccc3ccc21] | 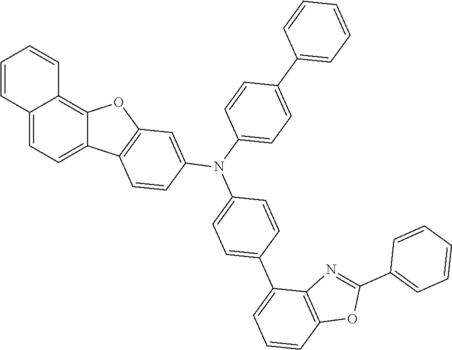 [SMILES: c1ccc(-c2ccc(N(c3ccc(-c4cccc5oc(-c6ccccc6)nc45)cc3)c3ccc4c(c3)oc3c5ccccc5ccc43)cc2)cc1] | 76% |
| | N30-B | N18-4 | N-12 | |
| 15 | 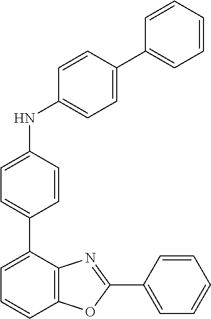 [SMILES: c1ccc(-c2ccc(Nc3ccc(-c4cccc5oc(-c6ccccc6)nc45)cc3)cc2)cc1] | 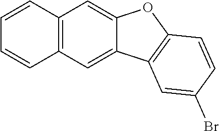 [SMILES: Brc1ccc2oc3cc4ccccc4cc3c2c1] | 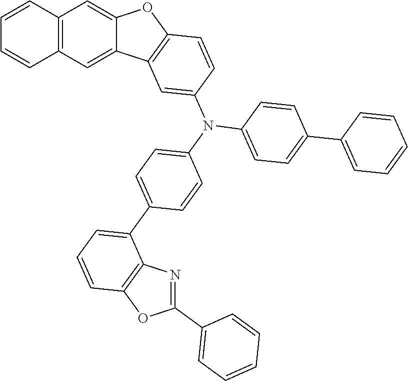 [SMILES: c1ccc(-c2ccc(N(c3ccc(-c4cccc5oc(-c6ccccc6)nc45)cc3)c3ccc4oc5cc6ccccc6cc5c4c3)cc2)cc1] | 80% |
| | N30-B | N21-4 | N-21 | |
| 16 | 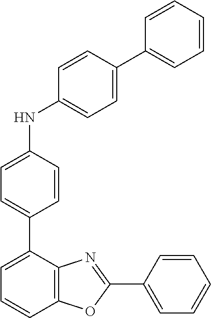 [SMILES: c1ccc(-c2ccc(Nc3ccc(-c4cccc5oc(-c6ccccc6)nc45)cc3)cc2)cc1] | 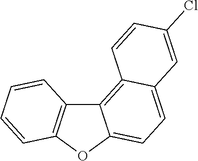 [SMILES: Clc1ccc2c(ccc3oc4ccccc4c32)c1] | 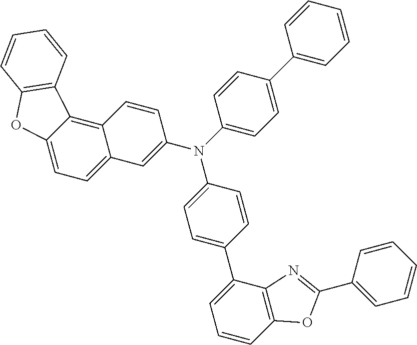 [SMILES: c1ccc(-c2ccc(N(c3ccc(-c4cccc5oc(-c6ccccc6)nc45)cc3)c3ccc4c(ccc5oc6ccccc6c54)c3)cc2)cc1] | 68% |
| | N30-B | N37-4 | N-37 | |
| 17 | 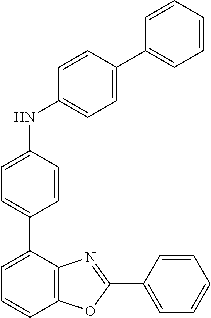 [SMILES: c1ccc(-c2ccc(Nc3ccc(-c4cccc5oc(-c6ccccc6)nc45)cc3)cc2)cc1] | 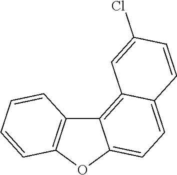 [SMILES: Clc1ccc2ccc3oc4ccccc4c3c2c1] | 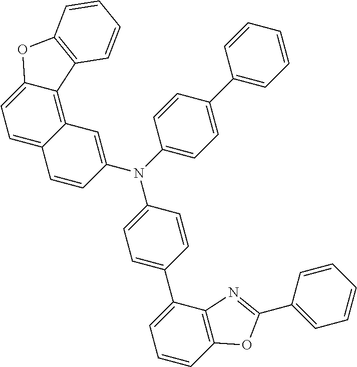 [SMILES: c1ccc(-c2ccc(N(c3ccc(-c4cccc5oc(-c6ccccc6)nc45)cc3)c3ccc4ccc5oc6ccccc6c5c4c3)cc2)cc1] | 65% |
| | N30-B | N44-4 | N-44 | |
| 18 | 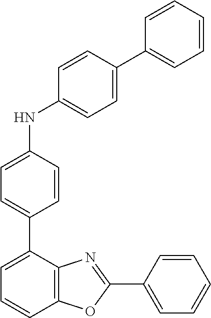 [SMILES: c1ccc(-c2ccc(Nc3ccc(-c4cccc5oc(-c6ccccc6)nc45)cc3)cc2)cc1] | 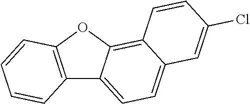 [SMILES: Clc1ccc2c(ccc3c4ccccc4oc23)c1] | 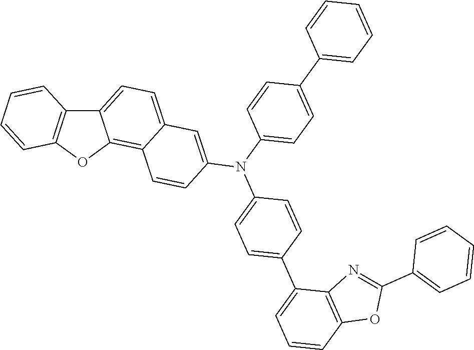 [SMILES: c1ccc(-c2ccc(N(c3ccc(-c4cccc5oc(-c6ccccc6)nc45)cc3)c3ccc4c(ccc5c6ccccc6oc45)c3)cc2)cc1] | 70% |
| | N30-B | N47-4 | N-47 | |
| 19 | 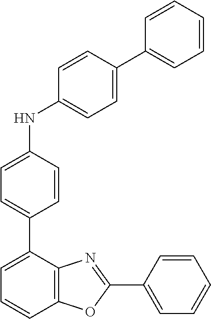 [SMILES: c1ccc(-c2ccc(Nc3ccc(-c4cccc5oc(-c6ccccc6)nc45)cc3)cc2)cc1] | 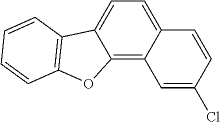 [SMILES: Clc1ccc2ccc3c4ccccc4oc3c2c1] | 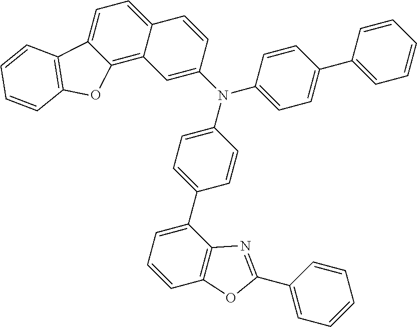 [SMILES: c1ccc(-c2ccc(N(c3ccc(-c4cccc5oc(-c6ccccc6)nc45)cc3)c3ccc4ccc5c6ccccc6oc5c4c3)cc2)cc1] | 68% |
| | N30-B | N52-4 | N-52 | |
| 20 | 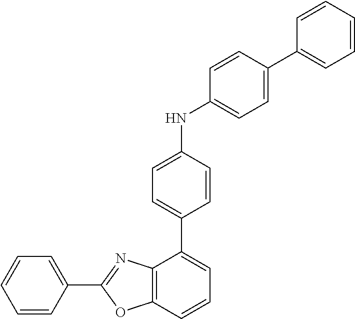 [SMILES: c1ccc(-c2ccc(Nc3ccc(-c4cccc5oc(-c6ccccc6)nc45)cc3)cc2)cc1] | 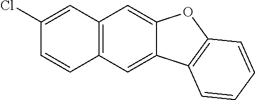 [SMILES: Clc1ccc2cc3c(cc2c1)oc1ccccc13] | 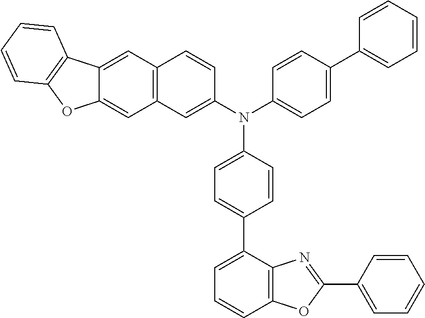 [SMILES: c1ccc(-c2ccc(N(c3ccc(-c4cccc5oc(-c6ccccc6)nc45)cc3)c3ccc4cc5c(cc4c3)oc3ccccc35)cc2)cc1] | 72% |
| | N30-B | N110-4 | N-110 | |
| 21 | 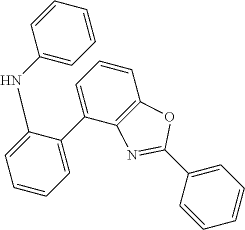 [SMILES: c1ccc(Nc2ccccc2-c2cccc3oc(-c4ccccc4)nc23)cc1] | 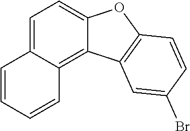 [SMILES: Brc1ccc2oc3ccc4ccccc4c3c2c1] | 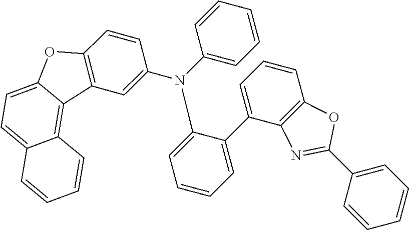 [SMILES: c1ccc(-c2nc3c(-c4ccccc4N(c4ccccc4)c4ccc5oc6ccc7ccccc7c6c5c4)cccc3o2)cc1] | 75% |
| | N156-B | N156-4 | N-156 | |

The characterization date of the products prepared in Examples 13-21 are shown in Table 2:

TABLE 2

| | | | HRMS (ESI) m/z | |
|---|---|---|---|---|
| | | | [M + H]+ | |
| Com- | Elemental analysis | | Theoretical | Measured |
| pound | Theoretical Value | Measured Value | Value | Value |
| N-3 | C, 86.22; H, 4.62; | C, 86.24; H, 4.61; | 654.23 | 655.16 |
| | N, 4.28; O, 4.89 | N, 4.27 | | |
| N-12 | C, 86.22; H, 4.62; | C, 86.23; H, 4.62; | 654.23 | 655.32 |
| | N, 4.28; O, 4.89 | N, 4.27 | | |
| N-21 | C, 86.22; H, 4.62; | C, 86.24; H, 4.62; | 654.23 | 655.35 |
| | N, 4.28; O, 4.89 | N, 4.26 | | |
| N-37 | C, 86.22; H, 4.62; | C, 86.20; H, 4.63; | 654.23 | 654.78 |
| | N, 4.28; O, 4.89 | N, 4.29 | | |
| N-44 | C, 86.22; H, 4.62; | C, 86.25; H, 4.60; | 654.23 | 654.98 |
| | N, 4.28; O, 4.89 | N, 4.27 | | |
| N-47 | C, 86.22; H, 4.62; | C, 86.24; H, 4.61; | 654.23 | 654.93 |
| | N, 4.28; O, 4.89 | N, 4.27 | | |
| N-52 | C, 86.22; H, 4.62; | C, 86.20; H, 4.63; | 654.23 | 654.82 |
| | N, 4.28; O, 4.89 | N, 4.29 | | |
| N-110 | C, 86.22; H, 4.62; | C, 86.21; H, 4.61; | 654.23 | 654.97 |
| | N, 4.28; O, 4.89 | N, 4.28 | | |
| N-156 | C, 85.10; H, 4.53; | C, 85.12; H, 4.55; | 578.20 | 579.26 |
| | N, 4.84; O, 5.53 | N, 4.80 | | |

### Example 22

This example provides a compound M-17 in an organic material composition, and its preparation method comprises the following steps:

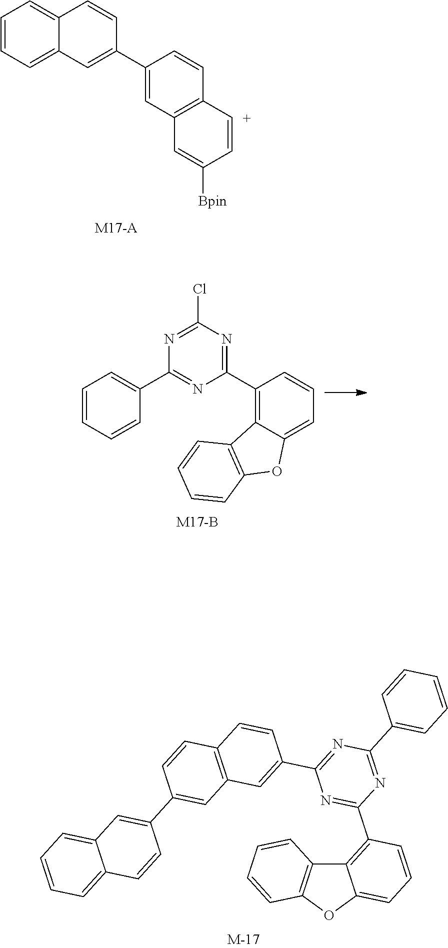

A 50 ml double-necked round-bottom flask was used, a stirring bar was put into it, connected to a reflux tube, then dried, filled with nitrogen, compounds M17-A (19.8 mmol, CAS: 1884145-03-2), M17-B (20.75 mmol, CAS: 1883265-32-4), tetrakistriphenylphosphine palladium (0.396 mmol), potassium carbonate (39.6 mmol), 35 ml of toluene, 15 ml of ethanol and 15 ml of distilled water were added to obtain a mixture, and the mixture was stirred at 90 degrees Celsius for 8 hours. After the reaction was completed, the mixture was added dropwise to methanol, and the obtained solid was filtered. The obtained solid was purified by column chromatography to obtain compound M-17 (8.5 g, yield: 75%).

Elemental analysis: C₄₁H₂₅N₃O; theoretical value: C, 85.54; H, 4.38; N, 7.30; O, 2.78; measured value: C, 85.52; H, 4.38; N, 7.32; HRMS (ESI) m/z (M+): theoretical value: 575.20; measured value: 576.34.

### Example 23

This example provides a compound M-296 in an organic material composition, and its preparation method comprises the following steps: The synthesis route of intermediate M296-A is as follows:

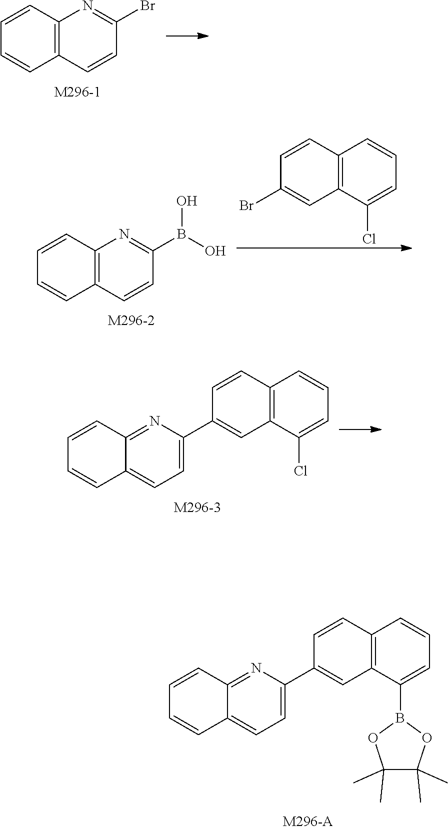 [SMILES: Brc1ccc2ccccc2n1 ; OB(O)c1ccc2ccccc2n1 ; Clc1cccc2ccc(Br)cc12 ; Clc1cccc2ccc(-c3ccc4ccccc4n3)cc12 ; CC1(C)OB(c2cccc3ccc(-c4ccc5ccccc5n4)cc23)OC1(C)C]

To a 250 mL three port bottle equipped with a thermometer and magnetic stirring, intermediate M296-1 (2-bromoquinoline, CAS: 2005-43-8, 20 g) and 200 mL anhydrous tetrahydrofuran were added. Under nitrogen protection, it was cooled to −78° C., the temperature was controlled and n-butyl lithium (1.6M, 45.2 mL) was dropwise added, stirred for 1 hour, then the temperature was controlled to −78° C. and triisopropyl borate (19.52 g) was added, transferred to room temperature and was reacted for 12 h, hydrochloric acid solution (6.5 mL of 36% hydrochloric acid+24 mL of water) was added, 50 mL of ethyl acetate was added to the reaction solution, extracted with 25 mL of water, the organic phase was spin dried and 50 mL of n-hexane was added, refluxed for 1 hour to produce a slurry, the slurry was filtered at room temperature and dried to obtain intermediate M296-2, 15 g.

To a 250 mL three port bottle equipped with a thermometer and magnetic stirring, intermediate M296-2 (15 g), intermediate 7-bromo-1-chloronaphthalene (21.9 g), potassium carbonate (16.6 g) and tetrakistriphenylphosphine palladium (2.0 g) were added, toluene (80 mL), ethanol (35 mL) and water (35 mL) were added and heated to 85° C. under nitrogen protection for 6 hours. 50 mL of ethyl acetate and 25 mL of water were added to the reaction solution for extraction and separation. The organic phase was mixed and passed through a column to obtain intermediate M296-3, 15 g.

To a 250 mL three port bottle equipped with a thermometer and magnetic stirring, intermediate M296-3 (15 g), bis(pinacolato)diboron (15.8 g), potassium acetate (10 g) and Pd(dppf)Cl₂ (0.64 g) were added, 1,4-dioxane (150 mL) was added, and heated to 110° C. and reacted for 4 hours under nitrogen protection. 100 mL of toluene and 100 mL of water were added to the reaction solution for extraction and separation. The organic phase was mixed and passed through a column to obtain intermediate M296-A, 16 g.

The synthesis route of compound M-296 is as follows:

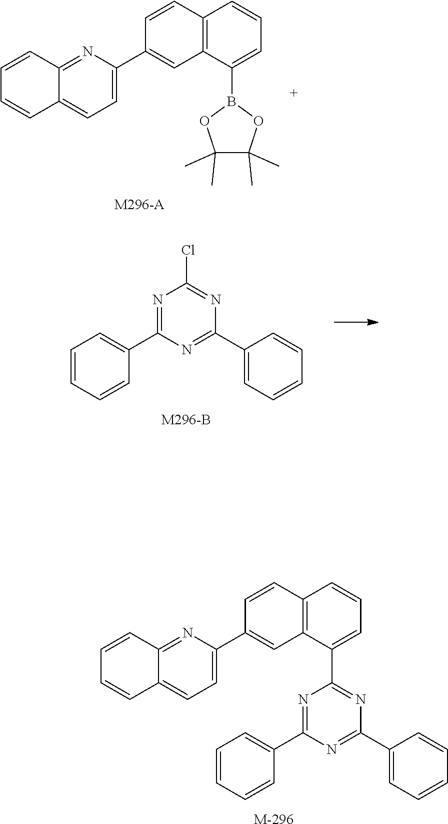

To a 250 mL three port bottle equipped with a thermometer and magnetic stirring, intermediate M296-A (16 g), intermediate M296-B (2-chloro-4,6-diphenyl-1,3,5-triazine, CAS: 3842-55-5, 11.2 g), potassium carbonate (11.6 g) and tetrakistriphenylphosphine palladium (1.3 g) were added, toluene (110 mL), ethanol (50 mL) and water (50 mL) were added under nitrogen protection, heated to 85° C. and reacted for 6 hours, water and ethanol were added at room temperature, the reaction solution was filtered, and dried to obtain the product M-296, 16 g (yield 78%).

Elemental analysis: C₃₄H₂₂N₄ theoretical value: C, 83.93; H, 4.56; N, 11.51; measured value: C, 83.95; H, 4.56; N, 11.49; HRMS (ESI) m/z (M+): theoretical value: 486.18; measured value: 487.12.

### Examples 24-27

Examples 24-27 provide methods for preparing compounds M-76, M-253, M-394 or M-480. The specific preparation methods are as follows:

To raw materials Mn—B, raw materials Mn-A, potassium carbonate and tetrakistriphenylphosphine palladium, toluene, ethanol and water were added under nitrogen protection conditions, and the temperature was raised to react. After the reaction was completed, the final product was purified; the amount of the substances used and the experimental parameters were the same as those in Example 1.

The structures and yields of raw material Mn—B, raw material Mn-A and products were shown in Table 3 below. The elemental analysis results of the prepared compound were shown in Table 4; the amount of substances used and the experimental parameters were the same as those in Example 22.

TABLE 3

| Ex- | | | | Yield |
|---|---|---|---|---|
| ample | Raw material Mn-A | Raw material Mn-B | Product M-n | % |
| 24 | 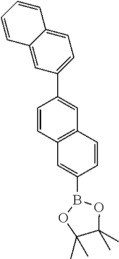 [SMILES: CC1(C)OB(c2ccc3cc(-c4ccc5ccccc5c4)ccc3c2)OC1(C)C] | 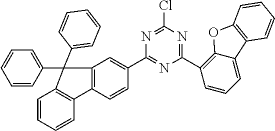 [SMILES: Clc1nc(-c2ccc3c(c2)C(c2ccccc2)(c2ccccc2)c2ccccc2-3)nc(-c2cccc3c2oc2ccccc23)n1] | 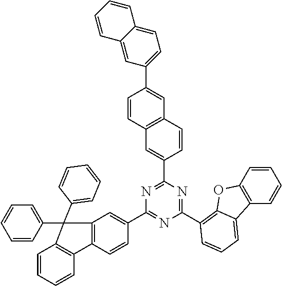 [SMILES: c1ccc(C2(c3ccccc3)c3ccccc3-c3ccc(-c4nc(-c5ccc6cc(-c7ccc8ccccc8c7)ccc6c5)nc(-c5cccc6c5oc5ccccc56)n4)cc32)cc1] | 76 |
| | M76-A | M76-B | M-76 | |
| 25 | 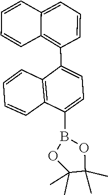 [SMILES: CC1(C)OB(c2ccc(-c3cccc4ccccc34)c3ccccc23)OC1(C)C] | 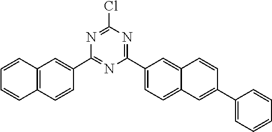 [SMILES: Clc1nc(-c2ccc3ccccc3c2)nc(-c2ccc3cc(-c4ccccc4)ccc3c2)n1] | 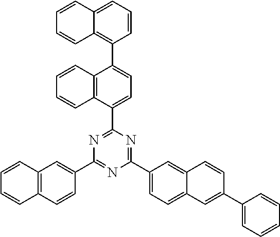 [SMILES: c1ccc(-c2ccc3cc(-c4nc(-c5ccc6ccccc6c5)nc(-c5ccc(-c6cccc7ccccc67)c6ccccc56)n4)ccc3c2)cc1] | 77 |
| | M253-A | M253-B | M-253 | |
| 26 | 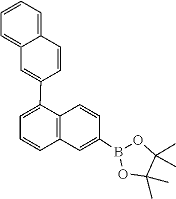 [SMILES: CC1(C)OB(c2ccc3c(-c4ccc5ccccc5c4)cccc3c2)OC1(C)C] | 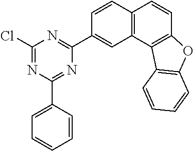 [SMILES: Clc1nc(-c2ccccc2)nc(-c2ccc3ccc4oc5ccccc5c4c3c2)n1] | 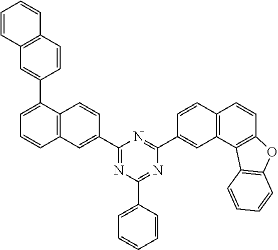 [SMILES: c1ccc(-c2nc(-c3ccc4c(-c5ccc6ccccc6c5)cccc4c3)nc(-c3ccc4ccc5oc6ccccc6c5c4c3)n2)cc1] | 79 |
| | M394-A | M394-B | M-394 | |
| 27 | 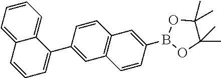 [SMILES: CC1(C)OB(c2ccc3cc(-c4cccc5ccccc45)ccc3c2)OC1(C)C] | 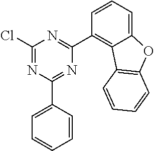 [SMILES: Clc1nc(-c2ccccc2)nc(-c2cccc3oc4ccccc4c23)n1] | 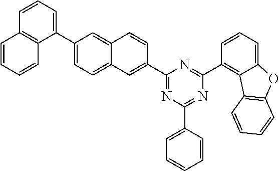 [SMILES: c1ccc(-c2nc(-c3ccc4cc(-c5cccc6ccccc56)ccc4c3)nc(-c3cccc4oc5ccccc5c34)n2)cc1] | 79 |
| | M480-A | M480-B | M-480 | |

The product characterization data are shown in Table 4:

TABLE 4

| | | | HRMS (ESI) m/z | |
|---|---|---|---|---|
| | | | [M + H]+ | |
| Com- | Elemental analysis | | Theoretical | Measured |
| pound | Theoretical Value | Measured Value | Value | Value |
| M-76 | C, 88.32; H, 4.57; | C, 88.30; H, 4.57; | 815.29 | 816.32 |
| | N, 5.15; O, 1.96; | N, 5.17; | | |
| M-253 | C, 88.93; H, 4.72; | C, 88.91; H, 4.73; | 661.25 | 662.40 |
| | N, 6.35; | N, 6.36; | | |
| M-394 | C, 86.38; H, 4.35; | C, 86.42; H, 4.34; | 625.22 | 626.42 |
| | N, 6.72; O, 2.56 | N, 6.68; | | |
| M-480 | C, 85.54; H, 4.38; | C, 85.50; H, 4.40; | 575.20 | 576.31 |
| | N, 7.30; O, 2.78 | N, 7.33; | | |

### Example 28

This example provides a compound M-620 in an organic material composition, and its preparation method comprises the following steps:

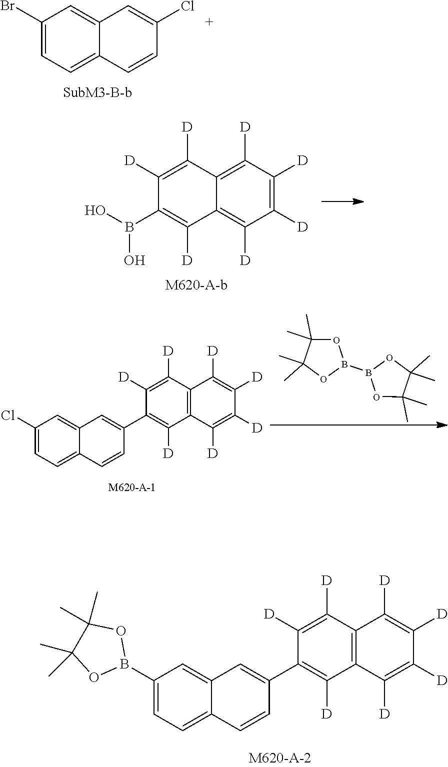

To a 250 mL three port bottle equipped with a thermometer and magnetic stirring, the raw material SubM3-B-b (42 mmol), the raw material deuterated 1-naphthaleneboronic acid (42.5 mmol), potassium carbonate (84 mmol) and tetrakistriphenylphosphine palladium (2 mmol) were added, toluene (80 mL), ethanol (35 mL) and water (35 mL) were added, and under nitrogen protection, heated to 85° C. and reacted for 6 hours. 50 mL of ethyl acetate and 25 mL of water were added to the reaction solution for extraction and separation. The organic phase was mixed and passed through a column to obtain the intermediate M620-A-1, 10 g.

To a 250 mL three port bottle equipped with a thermometer and magnetic stirring, intermediate M620-A-1 (34 mmol), bis(pinacolato)diboron (40 mmol), potassium acetate (68 mmol) and Pd(dppf)Cl₂ (1.7 mmol) were added, 1,4-dioxane (150 mL) was added under nitrogen protection, heated to 110° C. and reacted for 4 hours. 100 mL of toluene and 100 mL of water were added to the reaction solution for extraction and separation. The organic phase was mixed and passed through a column to obtain intermediate M620-A-2, 8 g.

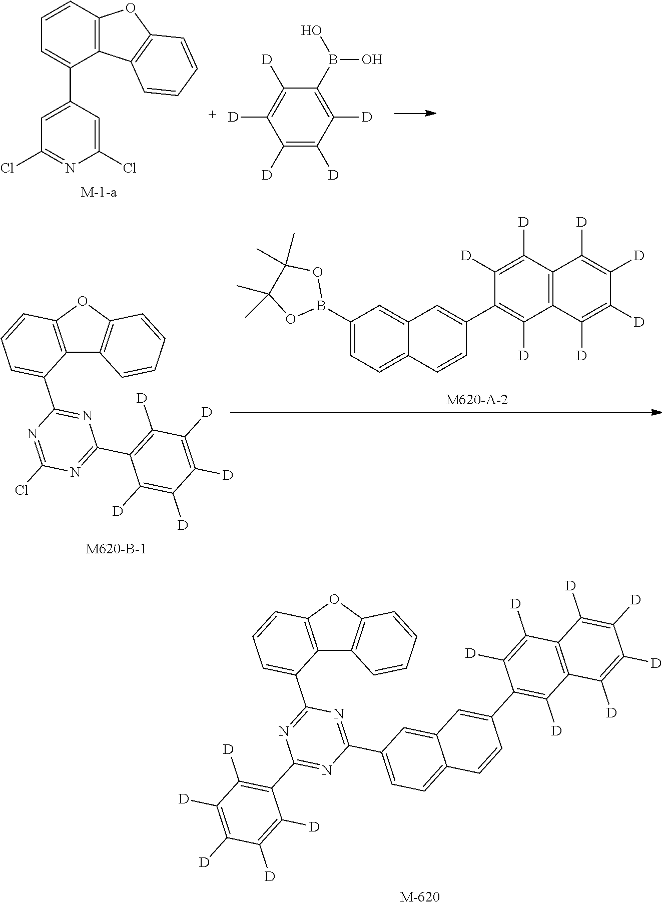 [SMILES: Clc1cc(-c2cccc3oc4ccccc4c23)cc(Cl)n1 ; [2H]c1c([2H])c([2H])c(B(O)O)c([2H])c1[2H] ; [2H]c1c([2H])c([2H])c(-c2nc(Cl)nc(-c3cccc4oc5ccccc5c34)n2)c([2H])c1[2H] ; [2H]c1c([2H])c([2H])c2c([2H])c(-c3ccc4ccc(B5OC(C)(C)C(C)(C)O5)cc4c3)c([2H])c([2H])c2c1[2H] ; [2H]c1c([2H])c([2H])c(-c2nc(-c3ccc4ccc(-c5c([2H])c([2H])c6c([2H])c([2H])c([2H])c([2H])c6c5[2H])cc4c3)nc(-c3cccc4oc5ccccc5c34)n2)c([2H])c1[2H] ; "M-620"]

After replacing the air in a three port reaction bottle equipped with mechanical stirring, thermometer, and condenser tube with nitrogen, M620-B-a (10 mmol), M-1-a (1.05 mmol), 1,4-dioxane 100 mL, water 30 mL, sodium carbonate (20 mmol), Pd(PPh₃)₄ (0.05 mmol) were added in sequence, and was heated to 70-80° C. and reacted for 3 hours. The temperature was lowered to 25-30° C., 100 mL of water and 100 mL of toluene was added, and the mixture was stirred and separated. The organic phases were combined, 7 g of anhydrous sodium sulfate was added to the organic phase, and the mixture was stirred and dried. The organic phase was filtered and concentrated (−0.08-0.09 MPa, 55-60° C.) until no liquid flowed out. 50 mL of a mixed solvent of dichloromethane and petroleum ether was added with stirring, cooled to 0-5° C., filtered, to obtain the intermediate M620-B-1 with a yield of 46%.

After replacing the air in a three port reaction bottle equipped with mechanical stirring, thermometer, and condenser tube with nitrogen, intermediate M620-B-1 (10 mmol), intermediate 1-A (10 mmol), potassium carbonate (20 mmol), Pd(PPh₃)₄ (0.05 mmol), 100 mL of 1,4-dioxane, and 30 mL of water were added in sequence, stirring was started, and heated to 70-75° C. for reaction for 3 hours. The temperature was lowered to 25-30° C., 100 mL of water and 100 mL of dichloromethane were added, and the mixture was stirred and separated. The aqueous phase was extracted once with 100 mL of dichloromethane, and the mixture was separated. The organic phases were combined, 7 g of anhydrous sodium sulfate was added to the organic phase, and the mixture was stirred and dried. The organic phase was filtered and concentrated (−0.08-0.09 MPa, 55-60° C.) until no liquid flowed out. 20 mL of petroleum ether was added with stirring, the temperature was lowered by 0-5° C., and the crude product was filtered to obtain the crude product.

The crude product was recrystallized from toluene to obtain the product M-620 with a yield of 54%.

Elemental analysis: C₄₁H₁₃D₁₂N₃O theoretical value: C, 83.79; H, 6.34; N, 7.15; O, 2.72; measured value: C, 83.77; H, 6.33; N, 7.18; HRMS(ESI) m/z [M+H]+: theoretical value: 587.28; measured value: 588.27.

### Example 29

This example provides a compound M-624 in an organic material composition, and its preparation method comprises the following steps:

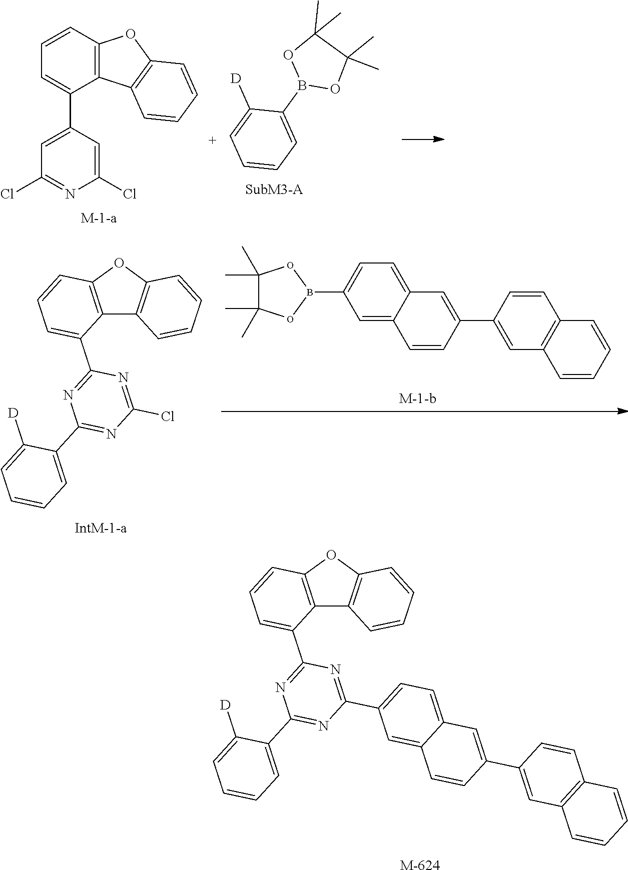

1) After replacing the air in a three port reaction bottle equipped with mechanical stirring, thermometer, and condenser tube with nitrogen, SubM3-A (10 mmol), M-1-a (1.05 mmol), 100 mL of 1,4-dioxane, 30 mL of water, sodium carbonate (20 mmol), Pd(PPh₃)₄ (0.05 mmol) were added in sequence, and was heated to 70-80° C. and reacted for 3 hours. The temperature was lowered to 25-30° C., 100 mL of water and 100 mL of toluene was added, and the mixture was stirred and separated. The aqueous phase was extracted once with 100 mL of toluene, and the liquid was separated. The organic phases were combined, 7 g of anhydrous sodium sulfate was added to the organic phase, and the mixture was stirred and dried. The organic phase was filtered and concentrated (−0.08-0.09 MPa, 55-60° C.) until no liquid flowed out. 50 mL of a mixed solvent of dichloromethane and petroleum ether was added with stirring, cooled to 0-5° C., filtered, to obtain the intermediate IntM-1-a with a yield of 43%.
2) After replacing the air in a three port reaction bottle equipped with mechanical stirring, thermometer, and condenser tube with nitrogen, intermediate IntM-1-a (10 mmol), intermediate 1-A (10 mmol), potassium carbonate (20 mmol), Pd(PPh₃)₄ (0.05 mmol), 100 mL of 1,4-dioxane, and 30 mL of water were added in sequence. Stirring was started, and heated to 70-75° C. for reaction for 3 hours. The temperature was lowered to 25-30° C., 100 mL of water and 100 mL of dichloromethane were added, and the mixture was stirred and separated. The aqueous phase was extracted once with 100 mL of dichloromethane, and the mixture was separated. The organic phases were combined, 7 g of anhydrous sodium sulfate was added to the organic phase, and the mixture was stirred and dried. The organic phase was filtered and concentrated (−0.08-0.09 MPa, 55-60° C.) until no liquid flowed out. 20 mL of petroleum ether was added with stirring, the temperature was lowered by 0-5° C., and the crude product was filtered to obtain the crude product. The crude product was recrystallized from toluene to obtain the product M-624 with a yield of 54%.

Elemental analysis: C₄₁H₂₄DN₃O theoretical value: C, 85.39; H, 4.54; N, 7.29; O, 2.77; measured value: C, 85.42; H, 4.55; N, 7.24; HRMS(ESI) m/z [M+H]+: theoretical value: 576.21; measured value: 577.32.

### Device Example

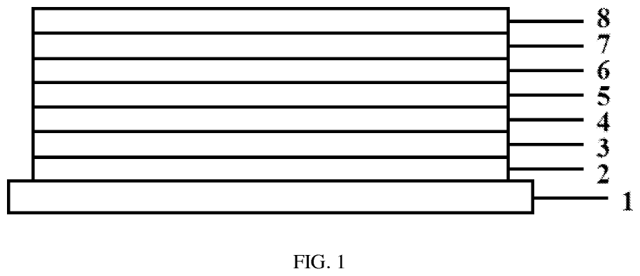

The present example provides an organic electroluminescent device, as shown in FIG. 1, comprising an anode 2, a hole injection layer 3, a hole transport layer 4, a light-emitting layer 5, an electron transport layer 6, an electron injection layer 7 and a cathode 8 stacked in sequence on a substrate 1, and the device structure is: anode (indium tin oxide (ITO) coated glass substrate)/hole injection layer (HIL)/hole transport layer (HTL)/light-emitting layer (EML)/electron transport layer (ETL)/electron injection layer (EIL)/cathode (Al).

The materials for manufacturing the organic electroluminescent device are as follows:

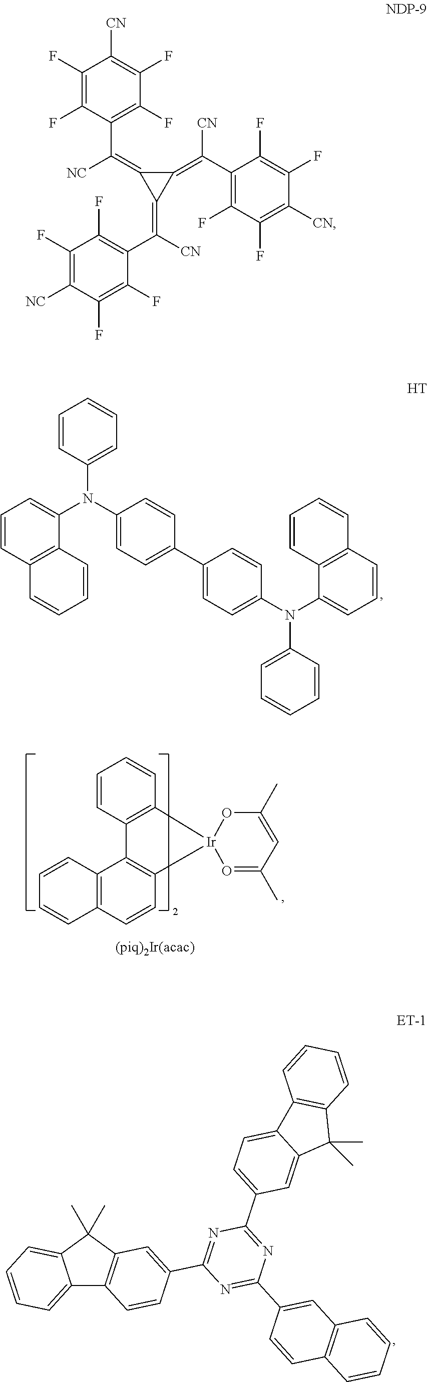

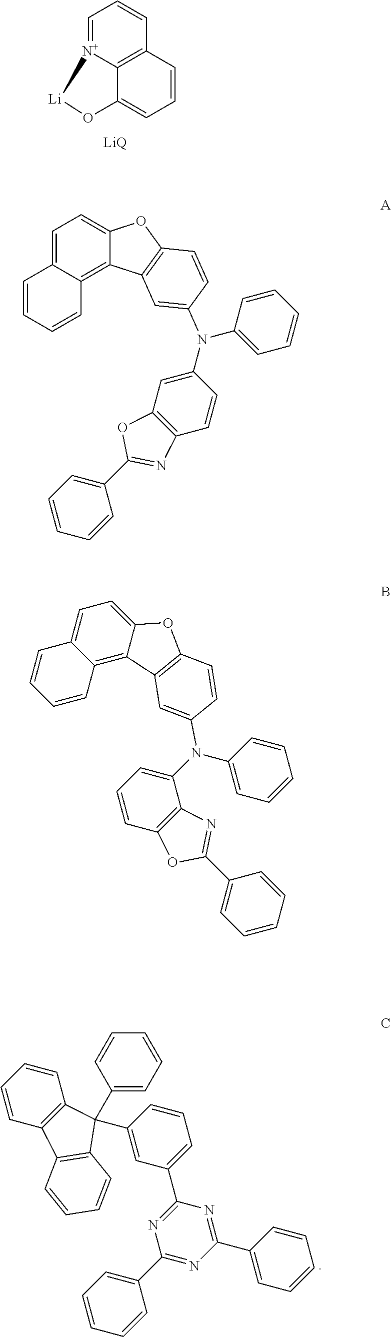

The preparation of the organic electroluminescent device comprises the following steps:

### 1) Substrate Cleaning:

The glass substrate coated with transparent ITO was ultrasonically treated in an aqueous cleaning agent (the composition and concentration of the aqueous cleaning agent: ethylene glycol solvent ≤10 wt %, triethanolamine ≥1 wt %), then rinsed in deionized water, ultrasonically degreased in a mixed solvent of acetone and ethanol (the volume ratio of acetone to ethanol is 1:1), baked in a clean environment until the moisture was completely removed, and then cleaned with ultraviolet light and ozone.

### 2) Preparation of Organic Layer:

The ITO transparent substrate was transferred to the evaporation equipment and evacuated to 1×10⁻⁶ to 2×10⁻⁴ Pa, and the hole injection layer (HIL)/hole transport layer (HTL)/light-emitting layer (EML)/electron transport layer (ETL)/electron injection layer (EIL)/thick cathode (Al) were sequentially evaporated on the anode film;
wherein the material of the hole injection layer (HIL) is a mixture of NDP-9 and HT, and the specific mass ratio is shown in Table 5;
The materials of the hole transport layer (HTL) are shown in Table 5;
The light-emitting layer (EML) is vacuum evaporated by co-evaporation. The material of the light-emitting layer includes a host material and a guest material, wherein the guest material is (piq)₂Ir(acac). The specific material of the host material and the ratio of the host material to the guest material are shown in Table 5.

The materials of the electron transport layer (ETL) are shown in Table 5;
The material of the electron injection layer (EIL) is LiQ;
The cathode is aluminum;
Some layers of the organic electroluminescent device and their materials and thicknesses are shown in Table 5:

TABLE 5

| | HIL | HTL | EML | ETL | EIL | Cathode |
|---|---|---|---|---|---|---|
| Example No. | thickness | thickness | thickness | thickness | thickness | thickness |
| Example 1 | NDP-9:HT(mass | HT | M-17:N-1:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 2 | NDP-9:HT(mass | HT | M-76:N-3:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 3 | NDP-9:HT(mass | HT | M-253:N-6:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 4 | NDP-9:HT(mass | HT | M-296:N-12:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 5 | NDP-9:HT(mass | HT | M-394:N-18:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 6 | NDP-9:HT(mass | HT | M-480:N-21:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 7 | NDP-9:HT(mass | HT | M-620:N-37:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 8 | NDP-9:HT(mass | HT | M-624:N-45:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 9 | NDP-9:HT(mass | HT | M-480:N-47:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 10 | NDP-9:HT(mass | HT | M-480:N-72:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 11 | NDP-9:HT(mass | HT | M-480:N-95:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 12 | NDP-9:HT(mass | HT | M-480:N-134:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 13 | NDP-9:HT(mass | HT | M-480:N-148:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 14 | NDP-9:HT(mass | HT | M-480:N-156:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 15 | NDP-9:HT(mass | HT | M-480:N-182:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Example 16 | NDP-9:HT(mass | HT | M-480:N-1:(piq)2Ir(acac) | ET-1:LiQ | LiQ | Al |
| | ratio 3:97) | 80 nm | (mass ratio 47.5:47.5:5) | (mass ratio | 1 nm | 80 nm |
| | 10 nm | | 38 nm | 1:1) 30 nm | | |
| Comparative | NDP-9:HT(mass | HT | M-17:(piq)2Ir(acac) | ET-1:LiQ | LiQ | Al |
| example 1 | ratio 3:97) | 80 nm | (mass ratio 95:5) | (mass ratio | 1 nm | 80 nm |
| | 10 nm | | 38 nm | 1:1) 30 nm | | |
| Comparative | NDP-9:HT(mass | HT | M-76:(piq)2Ir(acac) | ET-1:LiQ | LiQ | Al |
| example 2 | ratio 3:97) | 80 nm | (mass ratio 95:5) | (mass ratio | 1 nm | 80 nm |
| | 10 nm | | 38 nm | 1:1) 30 nm | | |
| Comparative | NDP-9:HT(mass | HT | M-480:(piq)2Ir(acac) | ET-1:LiQ | LiQ | Al |
| example 3 | ratio 3:97) | 80 nm | (mass ratio 95:5) | (mass ratio | 1 nm | 80 nm |
| | 10 nm | | 38 nm | 1:1) 30 nm | | |
| Comparative | NDP-9:HT(mass | HT | N-1:(piq)2Ir(acac) | ET-1:LiQ | LiQ | Al |
| example 4 | ratio 3:97) | 80 nm | (mass ratio 95:5) | (mass ratio | 1 nm | 80 nm |
| | 10 nm | | 38 nm | 1:1) 30 nm | | |
| Comparative | NDP-9:HT(mass | HT | N-21:(piq)2Ir(acac) | ET-1:LiQ | LiQ | Al |
| example 5 | ratio 3:97) | 80 nm | (mass ratio 95:5) | (mass ratio | 1 nm | 80 nm |
| | 10 nm | | 38 nm | 1:1) 30 nm | | |
| Comparative | NDP-9:HT(mass | HT | A:(piq)2Ir(acac) | ET-1:LiQ | LiQ | Al |
| example 6 | ratio 3:97) | 80 nm | (mass ratio 95:5) | (mass ratio | 1 nm | 80 nm |
| | 10 nm | | 38 nm | 1:1) 30 nm | | |
| Comparative | NDP-9:HT(mass | HT | B:(piq)2Ir(acac) | ET-1:LiQ | LiQ | Al |
| example 7 | ratio 3:97) | 80 nm | (mass ratio 47.5:47.5:5) | (mass ratio | 1 nm | 80 nm |
| | 10 nm | | 38 nm | 1:1) 30 nm | | |
| Comparative | NDP-9:HT(mass | HT | M-480:A:(piq)2Ir(acac) | ET-1:LiQ | LiQ | Al |
| example 8 | ratio 3:97) | 80 nm | (mass ratio 47.5:47.5:5) | (mass ratio | 1 nm | 80 nm |
| | 10 nm | | 38 nm | 1:1) 30 nm | | |
| Comparative | NDP-9:HT(mass | HT | M-480:B:(piq)2Ir(acac) | ET-1:LiQ | LiQ | Al |
| example 9 | ratio 3:97) | 80 nm | (mass ratio 47.5:47.5:5) | (mass ratio | 1 nm | 80 nm |
| | 10 nm | | 38 nm | 1:1) 30 nm | | |
| Comparative | NDP-9:HT(mass | HT | C:A:(piq)2Ir(acac) | ET-1:LiQ | LiQ | Al |
| example 10 | ratio 3:97) | 80 nm | (mass ratio 47.5:47.5:5) | (mass ratio | 1 nm | 80 nm |
| | 10 nm | | 38 nm | 1:1) 30 nm | | |
| Comparative | NDP-9:HT(mass | HT | C:B:(piq)2Ir(acac) | ET-1:LiQ | LiQ | Al |
| example 11 | ratio 3:97) | 80 nm | (mass ratio 47.5:47.5:5) | (mass ratio | 1 nm | 80 nm |
| | 10 nm | | 38 nm | 1:1) 30 nm | | |

The examples in the table represent device examples, and the comparative examples in the table are device comparative examples.

### Test Example

The organic electroluminescent devices obtained from device examples 1 to 16 and comparative examples 1 to 11 in the device examples were tested.
Instruments: The current, voltage, brightness and other characteristics of the device were tested synchronously using a PR 650 spectrum scanning brightness meter and a Keithley K2400 digital source meter system;
Test conditions: Photoelectric characteristics test conditions: Current density was 10 mA/cm².
Lifetime test: The current density was 50 mA/cm², and the time (in hours) when the device brightness drops to 95% of the original brightness was recorded.

The device performance test results are shown in Table 6:

TABLE 6

| | Driving voltage | Current efficiency | Lifetime T95 |
|---|---|---|---|
| No. | (V) | (Cd/A) | (hours) |
| Example1 | 3.22 | 35.68 | 350.7 |
| Example2 | 3.31 | 33.52 | 351.5 |
| Example3 | 3.33 | 32.58 | 342.1 |
| Example4 | 3.30 | 33.67 | 320.5 |
| Example5 | 3.38 | 31.28 | 320.8 |
| Example6 | 3.25 | 35.02 | 349.8 |
| Example7 | 3.20 | 35.10 | 359.6 |
| Example8 | 3.20 | 35.60 | 357.4 |
| Example9 | 3.32 | 33.51 | 325.0 |
| Example10 | 3.28 | 32.65 | 326.7 |
| Example11 | 3.28 | 32.52 | 322.4 |
| Example12 | 3.36 | 32.52 | 318.5 |
| Example13 | 3.25 | 34.62 | 335.5 |
| Example14 | 3.27 | 35.40 | 350.0 |
| Example15 | 3.22 | 35.27 | 350.5 |
| Example16 | 3.14 | 37.27 | 355.7 |
| Comparative | 3.72 | 25.11 | 140.20 |
| example1 | | | |
| Comparative | 3.79 | 25.69 | 139.70 |
| example2 | | | |
| Comparative | 3.75 | 25.89 | 153.50 |
| example3 | | | |
| Comparative | 3.90 | 14.55 | 62.30 |
| example4 | | | |
| Comparative | 3.92 | 12.11 | 41.50 |
| example5 | | | |
| Comparative | 4.05 | 8.65 | 22.70 |
| example6 | | | |
| Comparative | 4.01 | 10.35 | 35.40 |
| example7 | | | |
| Comparative | 3.70 | 28.69 | 293.5 |
| example8 | | | |
| Comparative | 3.62 | 30.89 | 315.0 |
| example9 | | | |
| Comparative | 3.94 | 16.41 | 99.65 |
| example10 | | | |
| Comparative | 3.92 | 17.52 | 96.84 |
| example11 | | | |

By comparing the data corresponding to the examples and comparative examples in Table 6, it can be seen that the organic material composition developed by the present invention has significantly better performance than the combination of compounds A, B, C disclosed in the prior art, and can have a lower turn-on voltage after being prepared into a device.

Obviously, the above examples are merely examples for clear description and are not limitations of the implementation methods. For those skilled in the art, other different forms of changes or modifications can be made based on the above description. It is not necessary and impossible to list all implementation methods exhaustively here. The obvious changes or modifications derived therefrom are still within the protection scope of the present invention.

1-13. (canceled)

14. An organic material composition, wherein the organic material composition comprises a first compound and a second compound, wherein the first compound has a structure shown in Formula (1): 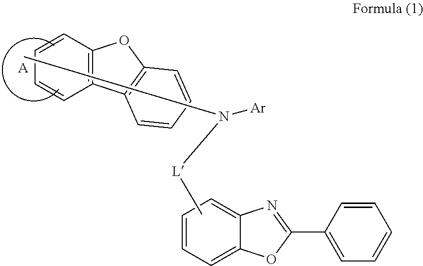
wherein ring A is a benzene ring;
Ar is selected from the group consisting of substituted or unsubstituted C6-C25 aryl;
L′ is selected from phenylene;
wherein the second compound has a structure shown in Formula (2): 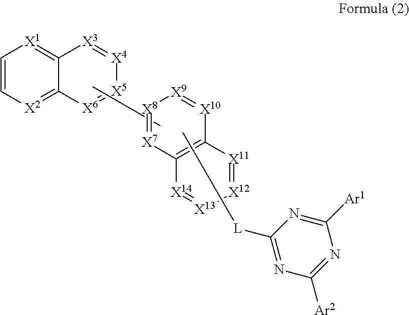
wherein X¹-X¹⁴ are each independently selected from N or CR, and R is selected from the group consisting of hydrogen, deuterium;
L is independently selected from a connecting bond;
Ar1 and Ar2 are each independently selected from unsubstituted C6-C25 aryl, unsubstituted C3-C20 heteroaryl;
the substituents in the substituted C6-C25 aryl is selected from the group consisting of deuterium and diphenylamine group.

15. The organic material composition according to claim 14, wherein in
Ar is selected from substituted or unsubstituted B group, and the B group is selected from the group consisting of phenyl, naphthyl, biphenyl, phenanthryl, fluoranthenyl, chrysenyl, terphenyl, triphenylenyl, phenalenyl, phenylnaphthyl, naphthylphenyl, phenylfluorenyl, and spirobifluorenyl.

16. The organic material composition according to claim 14, wherein Ar is selected from the group consisting of phenyl, naphthyl, biphenyl, chrysenyl, phenanthryl, terphenyl, phenylnaphthyl, naphthylphenyl, phenylfluorenyl, spirobifluorenyl, and phenalenyl.

17. The organic material composition according to claim 14, wherein the first compound has a structure shown in any one of the following below:

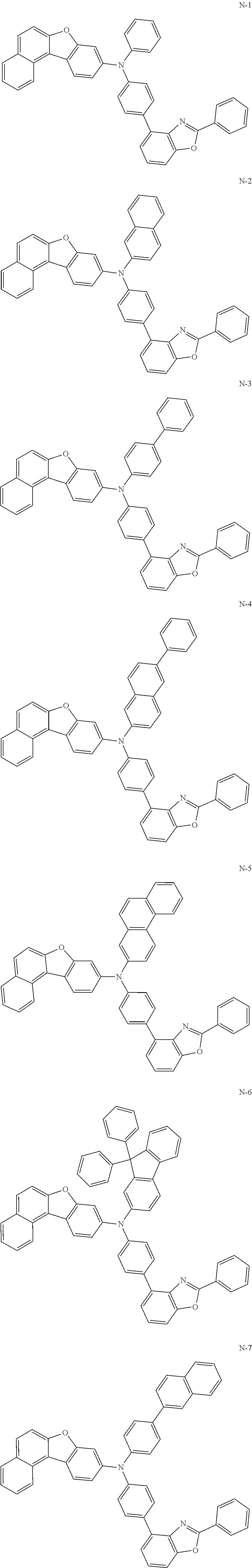

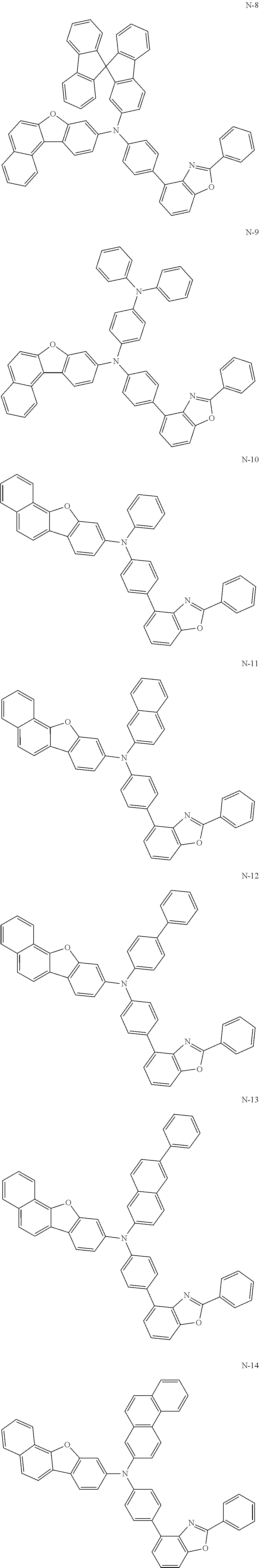

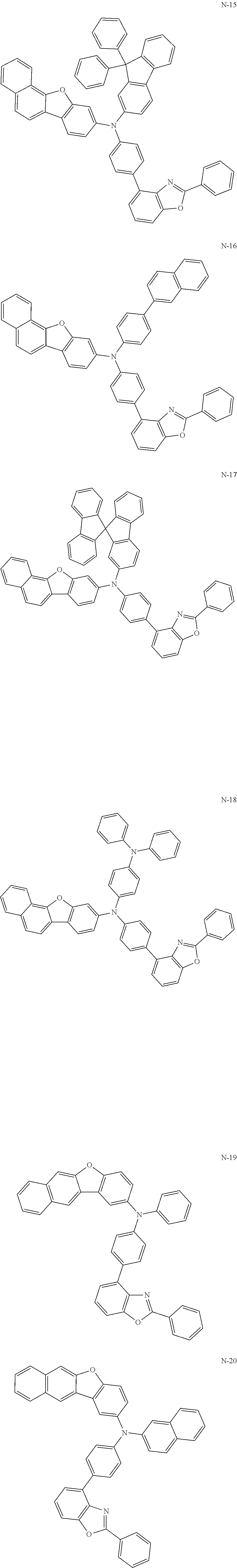

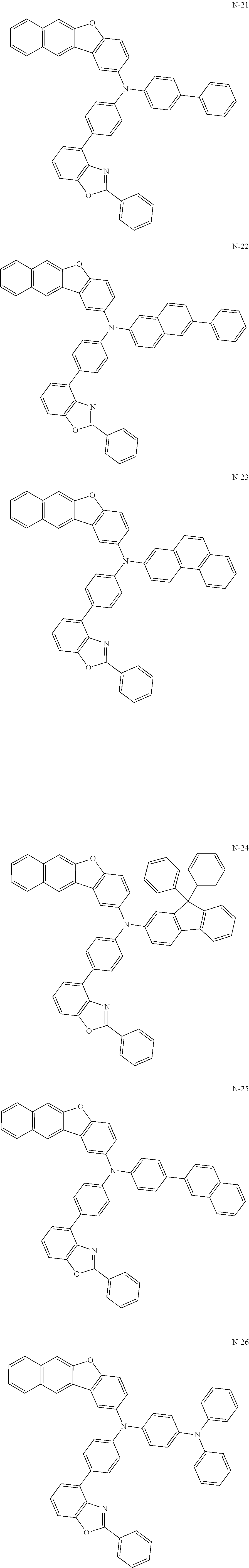

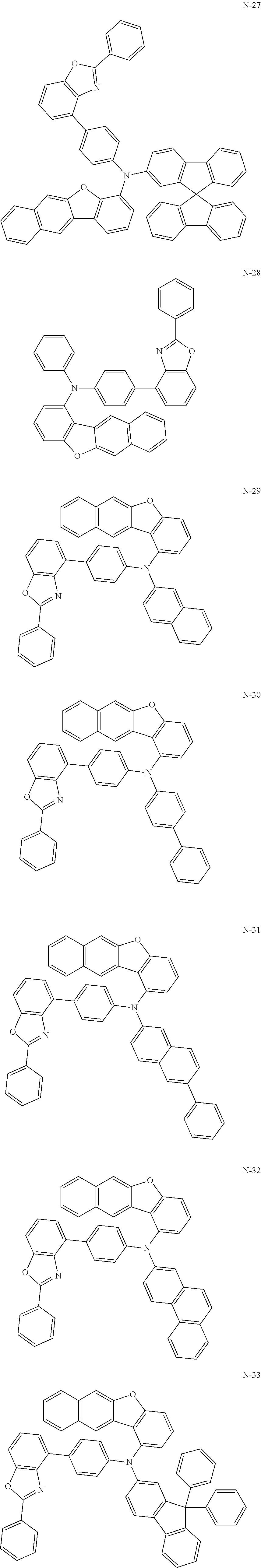

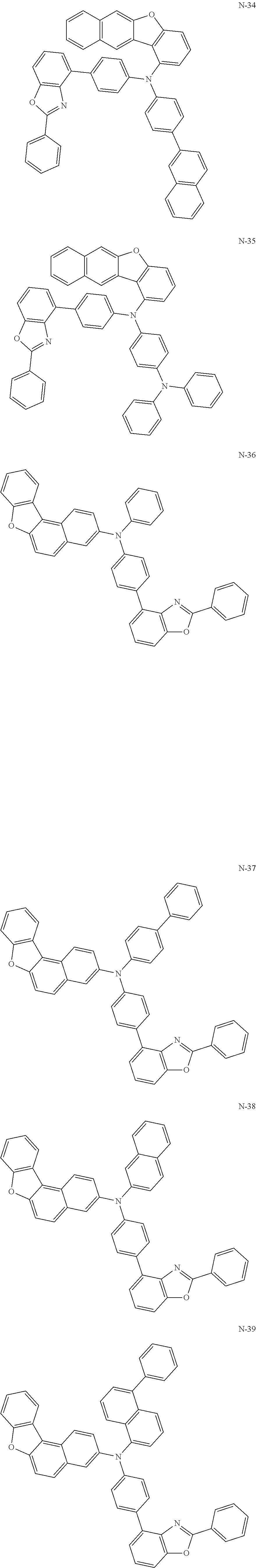

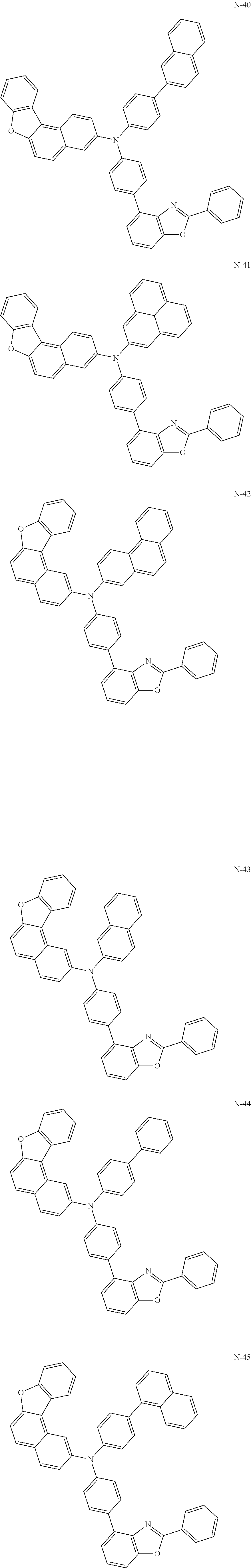

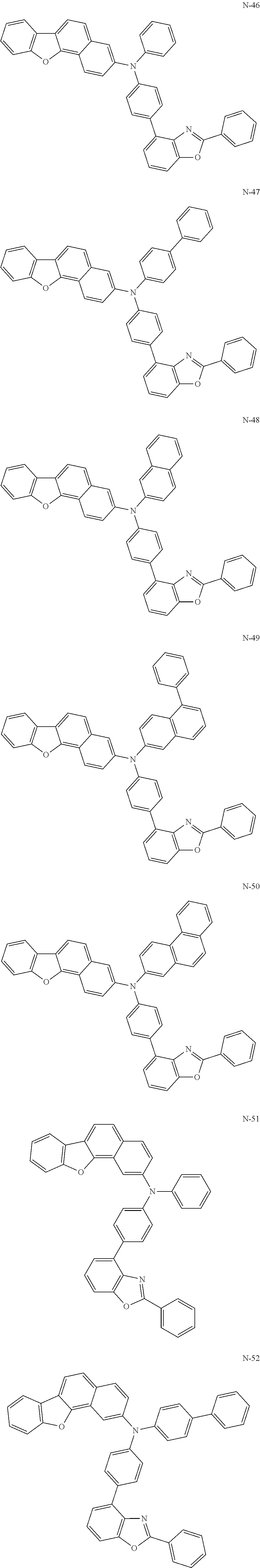

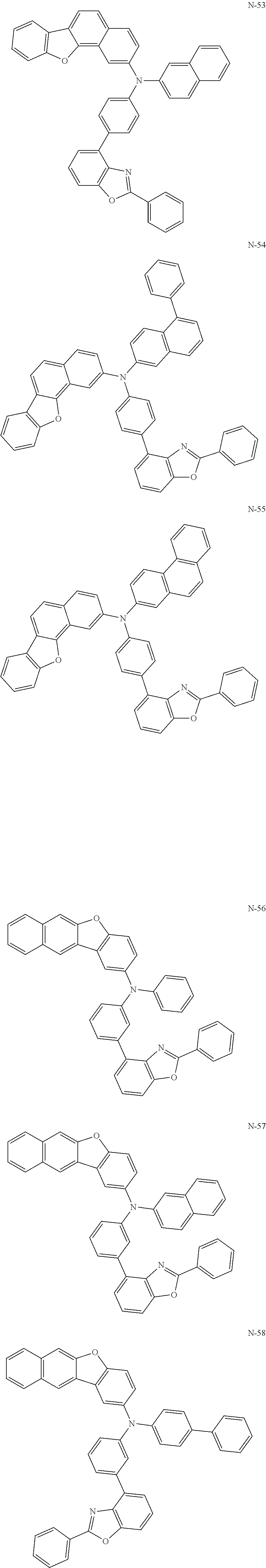

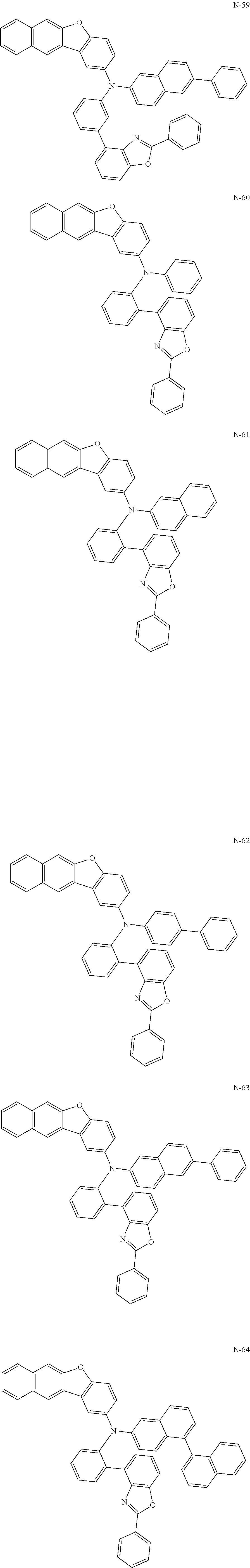

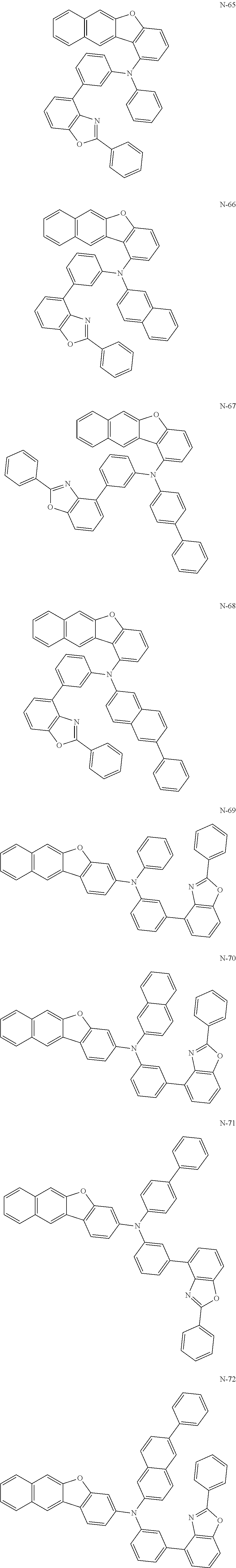

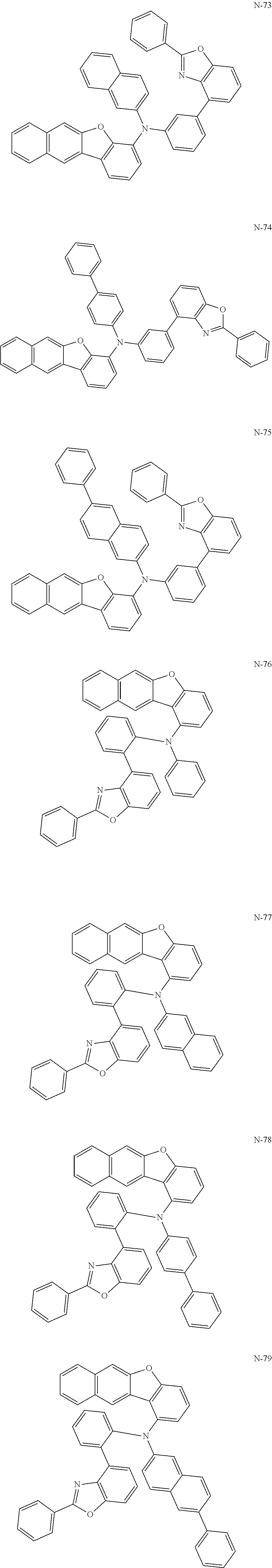

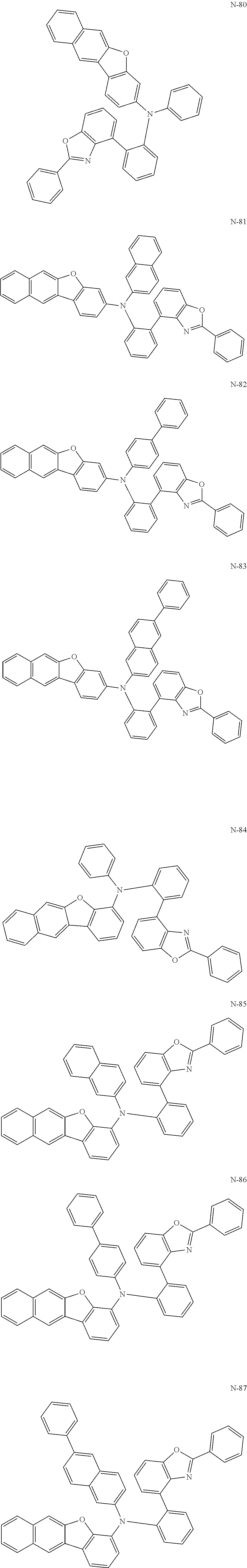

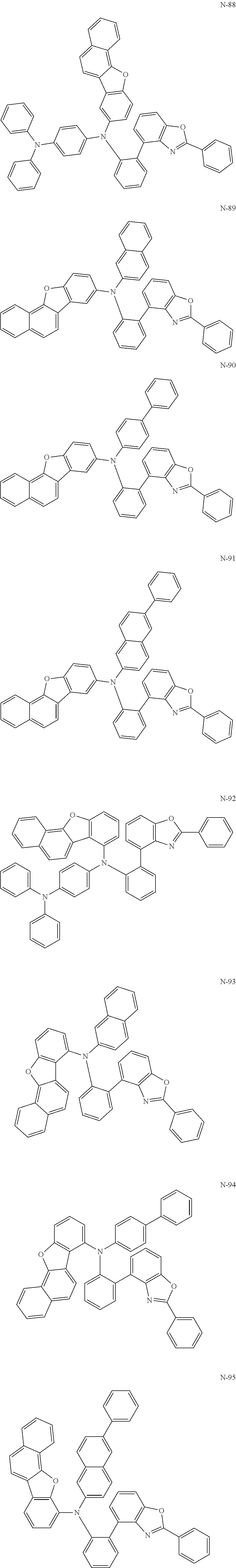

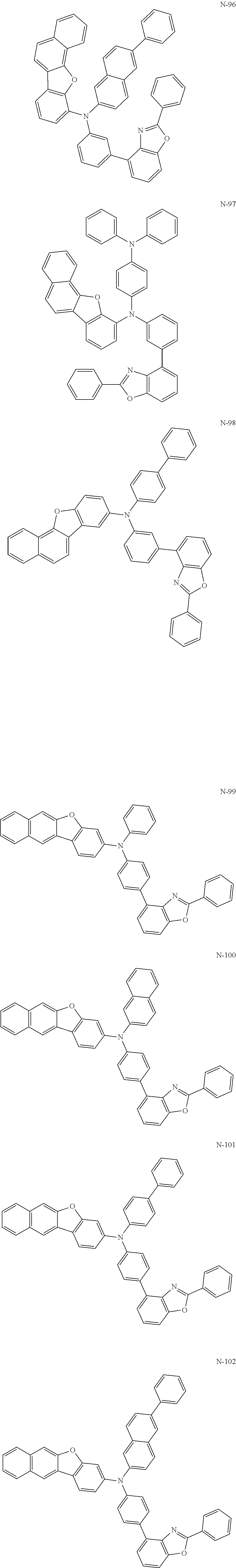

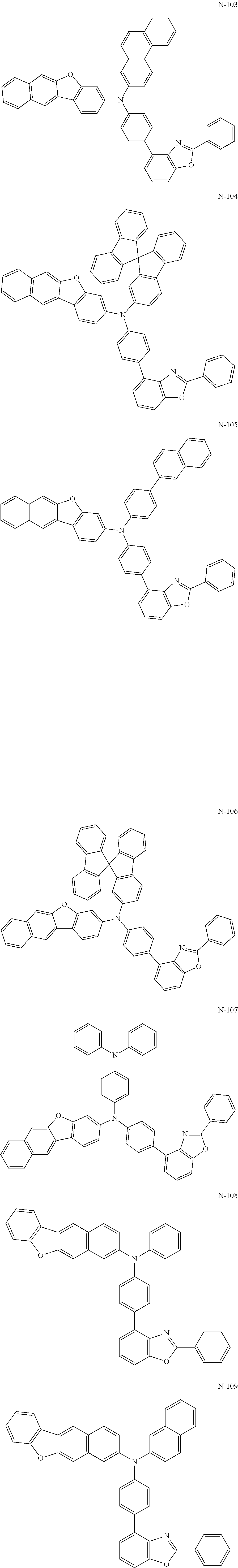

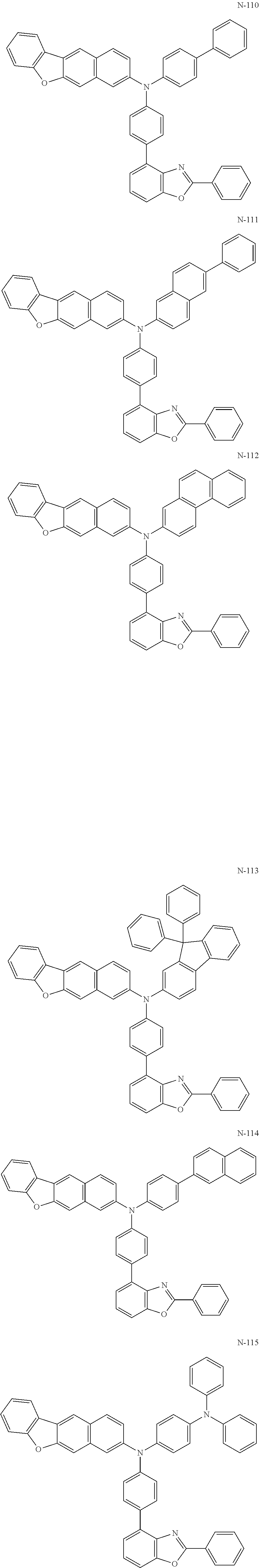

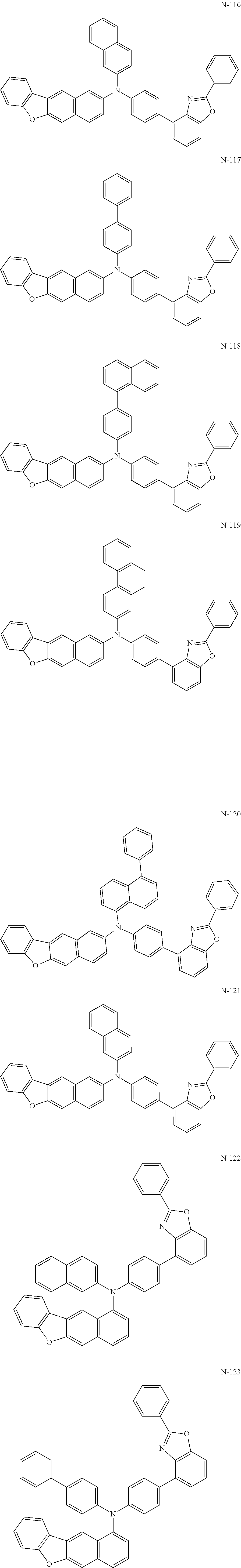

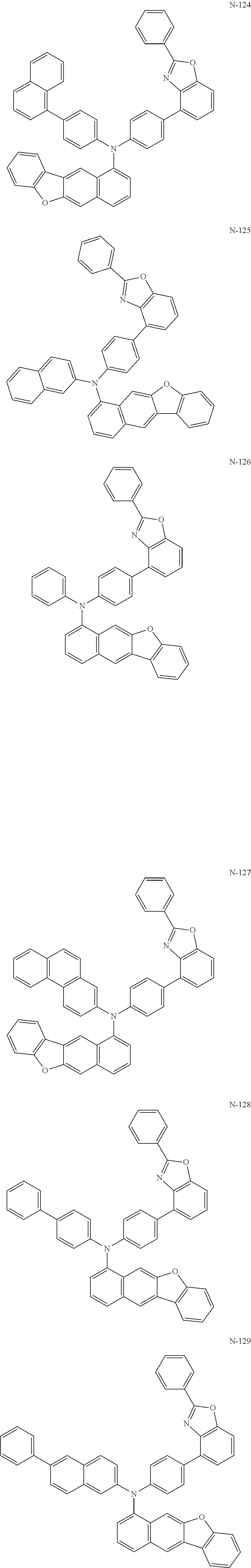

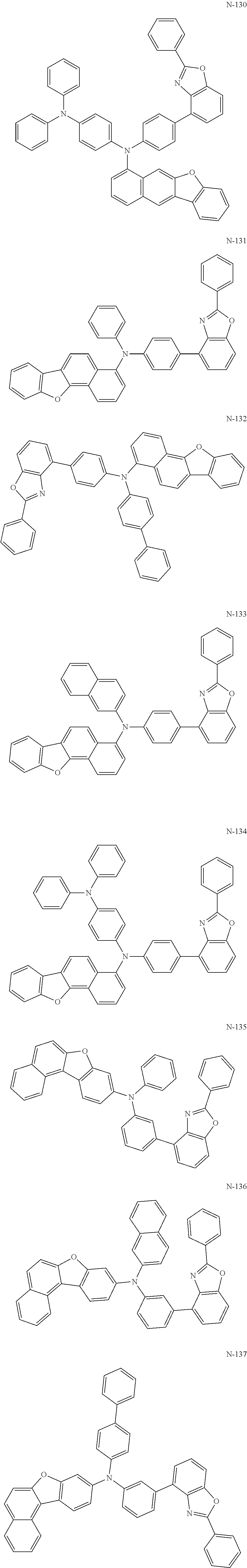

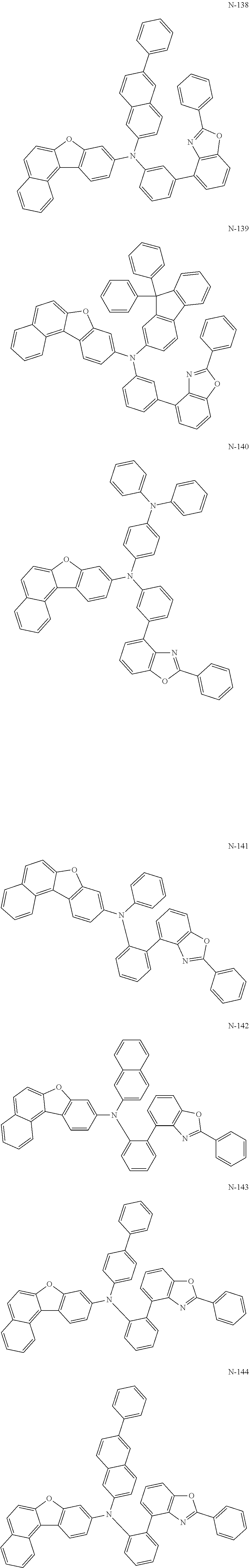

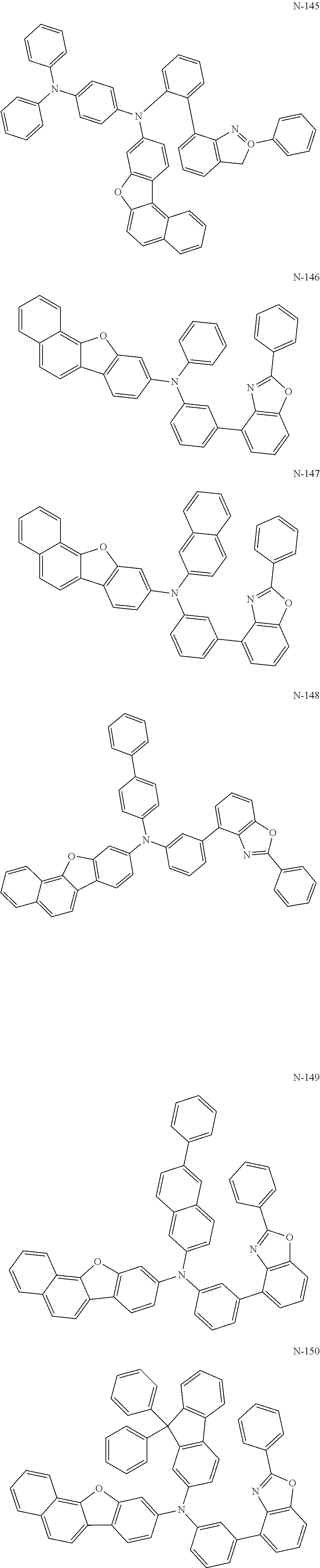

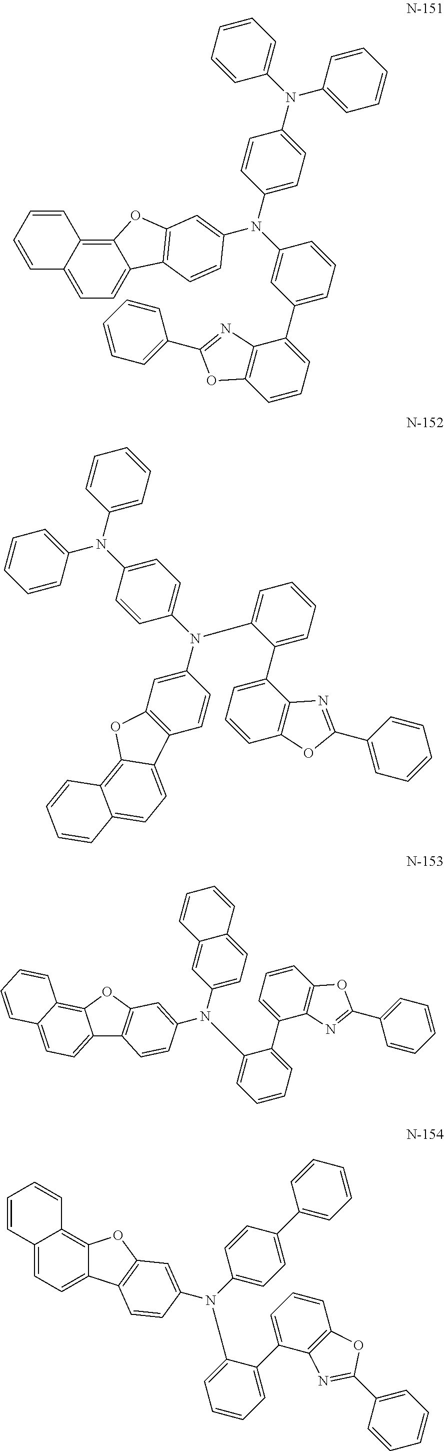 [SMILES: c1ccc(-c2nc3c(-c4cccc(N(c5ccc(N(c6ccccc6)c6ccccc6)cc5)c5ccc6c(c5)oc5c7ccccc7ccc65)c4)cccc3o2)cc1 ; c1ccc(-c2nc3c(-c4ccccc4N(c4ccc(N(c5ccccc5)c5ccccc5)cc4)c4ccc5c(c4)oc4c6ccccc6ccc54)cccc3o2)cc1 ; c1ccc(-c2nc3c(-c4ccccc4N(c4ccc5ccccc5c4)c4ccc5c(c4)oc4c6ccccc6ccc54)cccc3o2)cc1 ; c1ccc(-c2ccc(N(c3ccc4c(c3)oc3c5ccccc5ccc43)c3ccccc3-c3cccc4oc(-c5ccccc5)nc34)cc2)cc1]

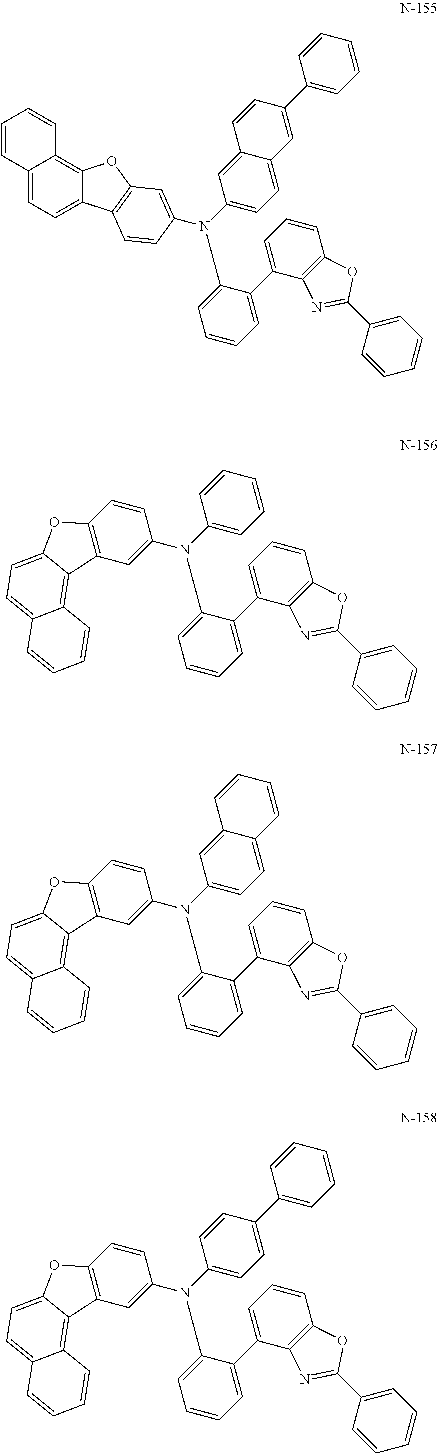 [SMILES: c1ccc(-c2ccc3cc(N(c4ccc5c(c4)oc4c6ccccc6ccc54)c4ccccc4-c4cccc5oc(-c6ccccc6)nc45)ccc3c2)cc1 ; c1ccc(-c2nc3c(-c4ccccc4N(c4ccccc4)c4ccc5oc6ccc7ccccc7c6c5c4)cccc3o2)cc1 ; c1ccc(-c2nc3c(-c4ccccc4N(c4ccc5ccccc5c4)c4ccc5oc6ccc7ccccc7c6c5c4)cccc3o2)cc1 ; c1ccc(-c2ccc(N(c3ccc4oc5ccc6ccccc6c5c4c3)c3ccccc3-c3cccc4oc(-c5ccccc5)nc34)cc2)cc1]

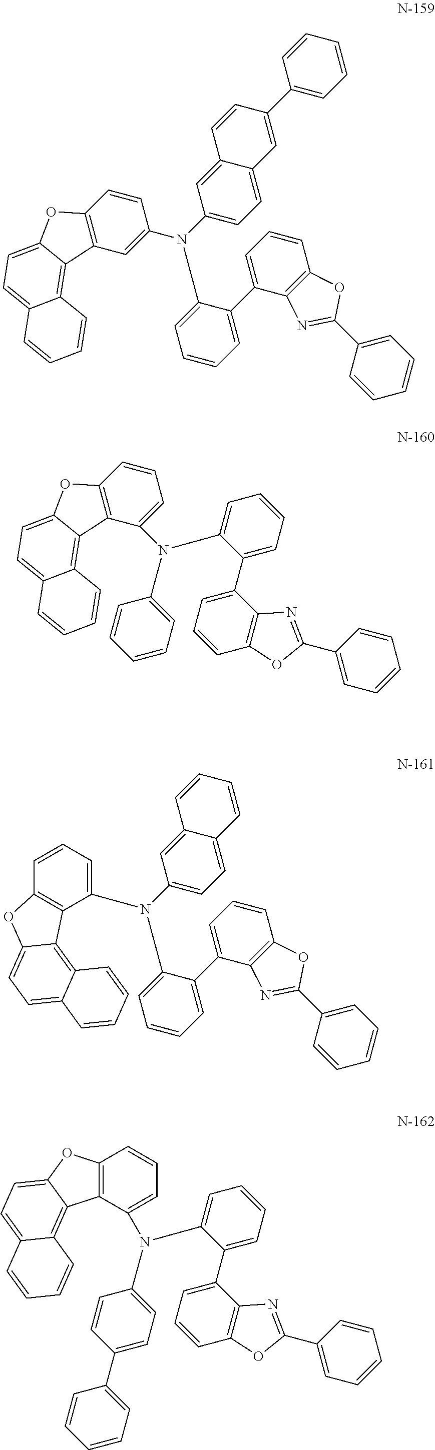 [SMILES: c1ccc(-c2ccc3cc(N(c4ccc5oc6ccc7ccccc7c6c5c4)c4ccccc4-c4cccc5oc(-c6ccccc6)nc45)ccc3c2)cc1 ; c1ccc(-c2nc3c(-c4ccccc4N(c4ccccc4)c4cccc5oc6ccc7ccccc7c6c45)cccc3o2)cc1 ; c1ccc(-c2nc3c(-c4ccccc4N(c4ccc5ccccc5c4)c4cccc5oc6ccc7ccccc7c6c45)cccc3o2)cc1 ; c1ccc(-c2ccc(N(c3ccccc3-c3cccc4oc(-c5ccccc5)nc34)c3cccc4oc5ccc6ccccc6c5c34)cc2)cc1]

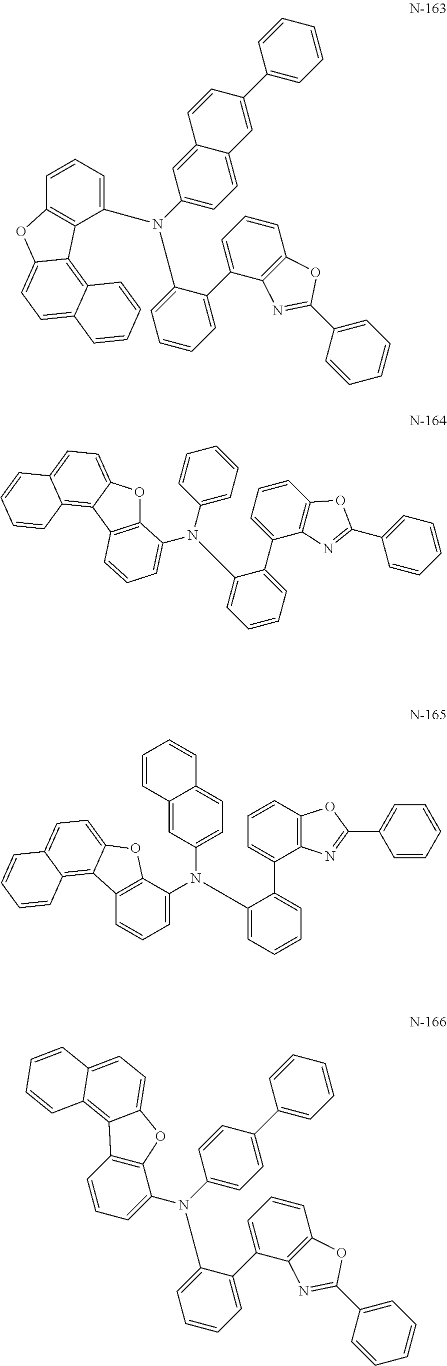 [SMILES: c1ccc(-c2ccc3cc(N(c4ccccc4-c4cccc5oc(-c6ccccc6)nc45)c4cccc5oc6ccc7ccccc7c6c45)ccc3c2)cc1 ; c1ccc(-c2nc3c(-c4ccccc4N(c4ccccc4)c4cccc5c4oc4ccc6ccccc6c45)cccc3o2)cc1 ; c1ccc(-c2nc3c(-c4ccccc4N(c4ccc5ccccc5c4)c4cccc5c4oc4ccc6ccccc6c45)cccc3o2)cc1 ; c1ccc(-c2ccc(N(c3ccccc3-c3cccc4oc(-c5ccccc5)nc34)c3cccc4c3oc3ccc5ccccc5c34)cc2)cc1]

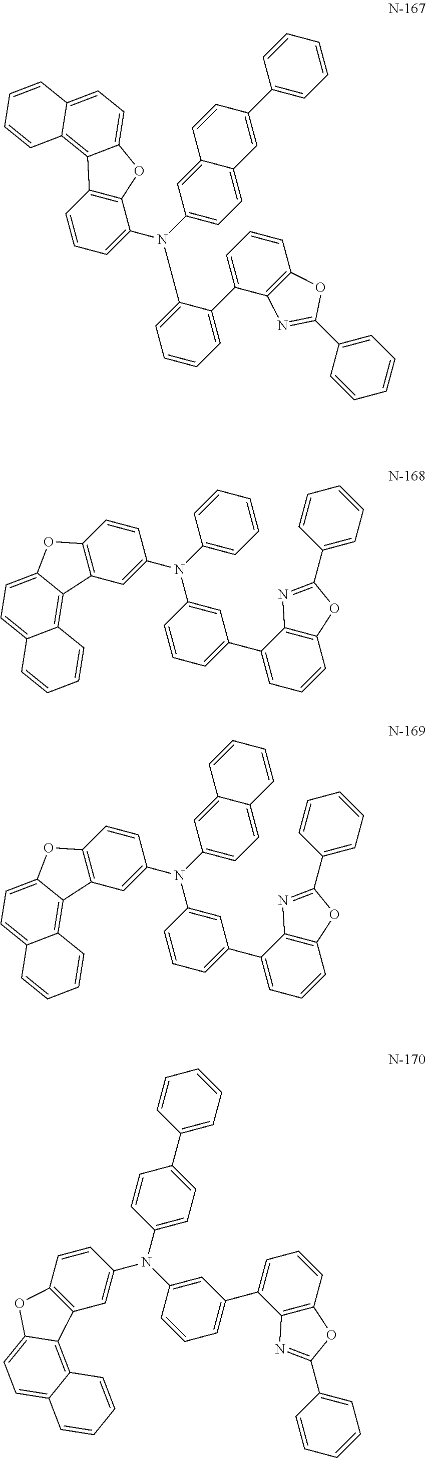 [SMILES: c1ccc(-c2ccc3cc(N(c4ccccc4-c4cccc5oc(-c6ccccc6)nc45)c4cccc5c4oc4ccc6ccccc6c45)ccc3c2)cc1 ; c1ccc(-c2nc3c(-c4cccc(N(c5ccccc5)c5ccc6oc7ccc8ccccc8c7c6c5)c4)cccc3o2)cc1 ; c1ccc(-c2nc3c(-c4cccc(N(c5ccc6ccccc6c5)c5ccc6oc7ccc8ccccc8c7c6c5)c4)cccc3o2)cc1 ; c1ccc(-c2ccc(N(c3cccc(-c4cccc5oc(-c6ccccc6)nc45)c3)c3ccc4oc5ccc6ccccc6c5c4c3)cc2)cc1]

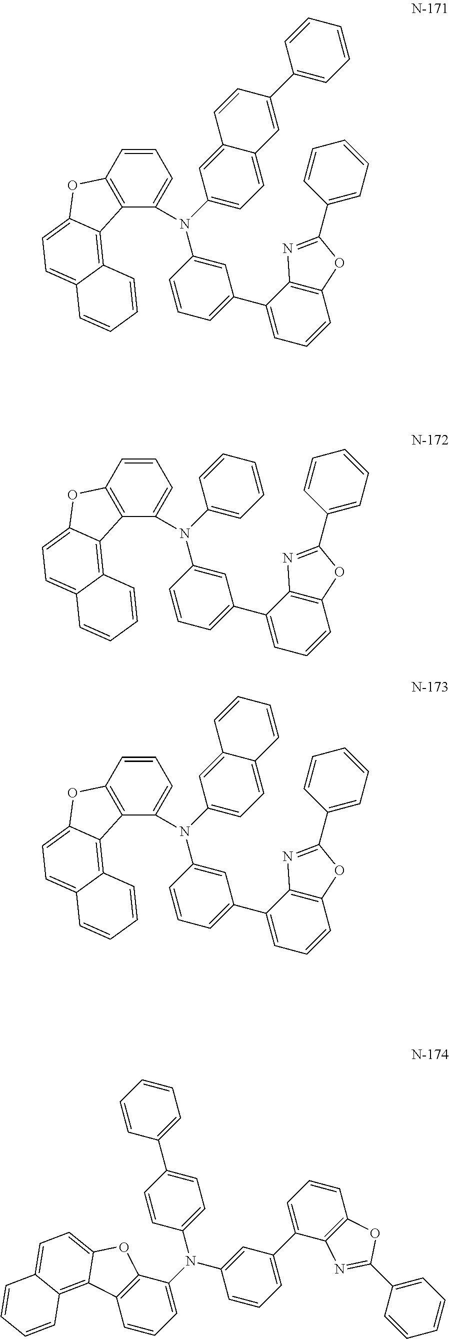 [SMILES: c1ccc(-c2ccc3cc(N(c4cccc(-c5cccc6oc(-c7ccccc7)nc56)c4)c4cccc5oc6ccc7ccccc7c6c45)ccc3c2)cc1 ; c1ccc(-c2nc3c(-c4cccc(N(c5ccccc5)c5cccc6oc7ccc8ccccc8c7c56)c4)cccc3o2)cc1 ; c1ccc(-c2nc3c(-c4cccc(N(c5ccc6ccccc6c5)c5cccc6oc7ccc8ccccc8c7c56)c4)cccc3o2)cc1 ; c1ccc(-c2ccc(N(c3cccc(-c4cccc5oc(-c6ccccc6)nc45)c3)c3cccc4c3oc3ccc5ccccc5c34)cc2)cc1]

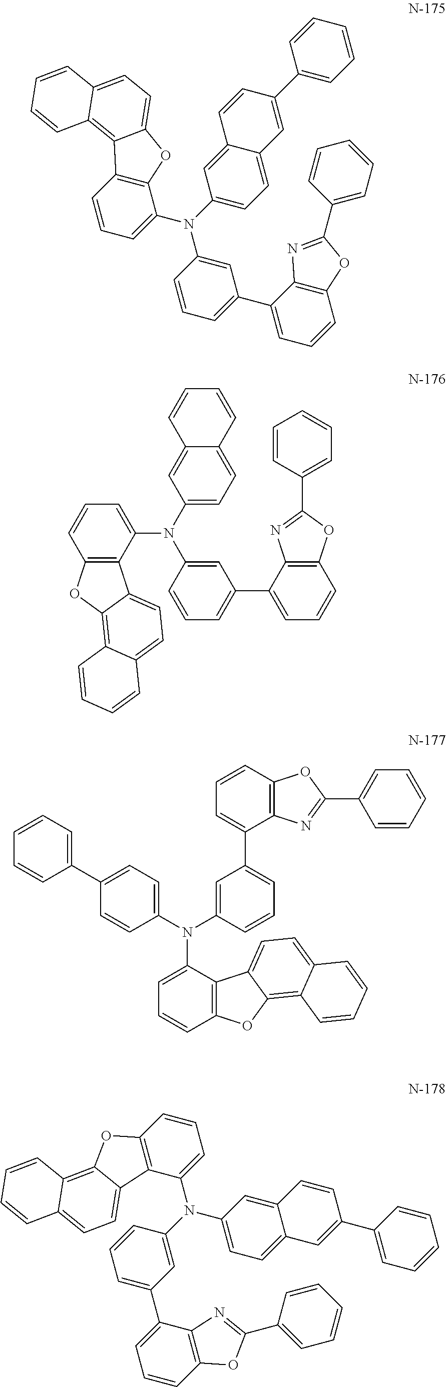 [SMILES: c1ccc(-c2ccc3cc(N(c4cccc(-c5cccc6oc(-c7ccccc7)nc56)c4)c4cccc5c4oc4ccc6ccccc6c45)ccc3c2)cc1 ; c1ccc(-c2nc3c(-c4cccc(N(c5ccc6ccccc6c5)c5cccc6oc7c8ccccc8ccc7c56)c4)cccc3o2)cc1 ; c1ccc(-c2ccc(N(c3cccc(-c4cccc5oc(-c6ccccc6)nc45)c3)c3cccc4oc5c6ccccc6ccc5c34)cc2)cc1 ; c1ccc(-c2ccc3cc(N(c4cccc(-c5cccc6oc(-c7ccccc7)nc56)c4)c4cccc5oc6c7ccccc7ccc6c45)ccc3c2)cc1]

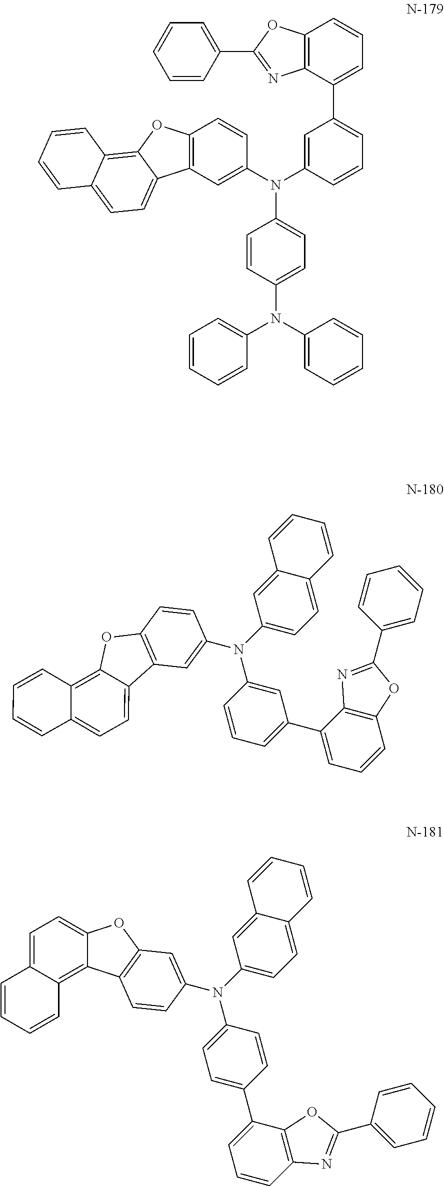 [SMILES: c1ccc(-c2nc3c(-c4cccc(N(c5ccc(N(c6ccccc6)c6ccccc6)cc5)c5ccc6oc7c8ccccc8ccc7c6c5)c4)cccc3o2)cc1 ; c1ccc(-c2nc3c(-c4cccc(N(c5ccc6ccccc6c5)c5ccc6oc7c8ccccc8ccc7c6c5)c4)cccc3o2)cc1 ; c1ccc(-c2nc3cccc(-c4ccc(N(c5ccc6ccccc6c5)c5ccc6c(c5)oc5ccc7ccccc7c56)cc4)c3o2)cc1]

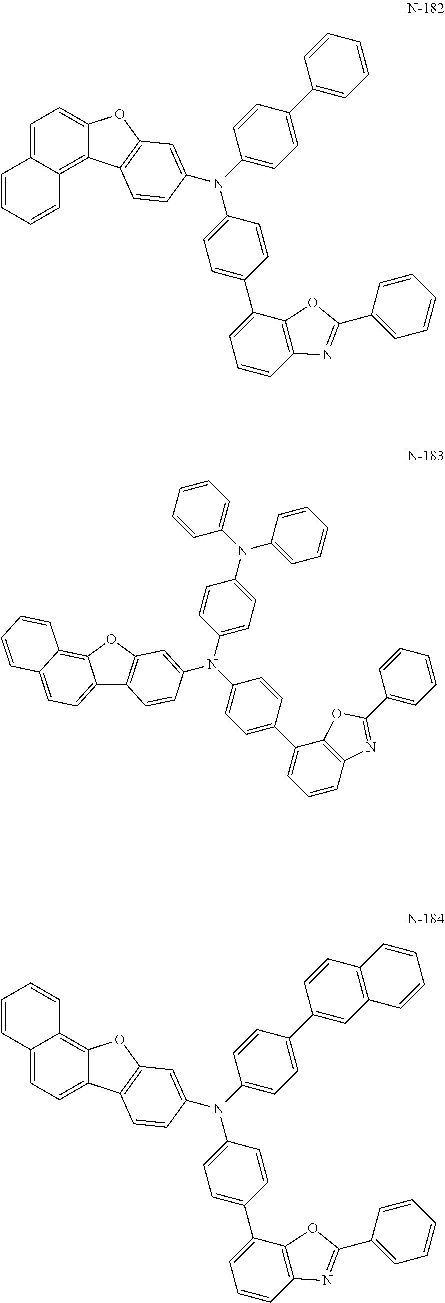 [SMILES: c1ccc(-c2ccc(N(c3ccc(-c4cccc5nc(-c6ccccc6)oc45)cc3)c3ccc4c(c3)oc3ccc5ccccc5c34)cc2)cc1 ; c1ccc(-c2nc3cccc(-c4ccc(N(c5ccc(N(c6ccccc6)c6ccccc6)cc5)c5ccc6c(c5)oc5c7ccccc7ccc65)cc4)c3o2)cc1 ; c1ccc(-c2nc3cccc(-c4ccc(N(c5ccc(-c6ccc7ccccc7c6)cc5)c5ccc6c(c5)oc5c7ccccc7ccc65)cc4)c3o2)cc1]

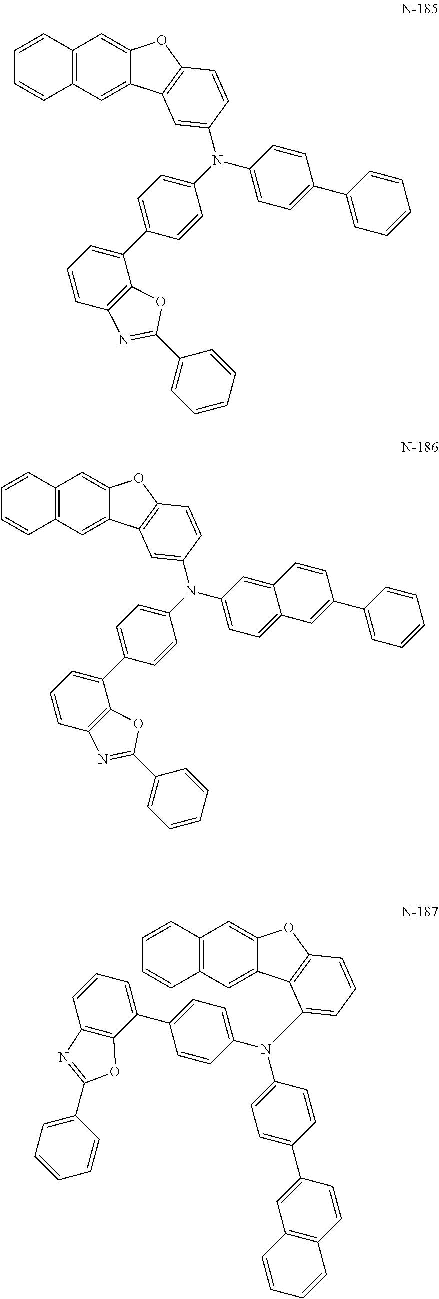 [SMILES: c1ccc(-c2ccc(N(c3ccc(-c4cccc5nc(-c6ccccc6)oc45)cc3)c3ccc4oc5cc6ccccc6cc5c4c3)cc2)cc1 ; c1ccc(-c2ccc3cc(N(c4ccc(-c5cccc6nc(-c7ccccc7)oc56)cc4)c4ccc5oc6cc7ccccc7cc6c5c4)ccc3c2)cc1 ; c1ccc(-c2nc3cccc(-c4ccc(N(c5ccc(-c6ccc7ccccc7c6)cc5)c5cccc6oc7cc8ccccc8cc7c56)cc4)c3o2)cc1]

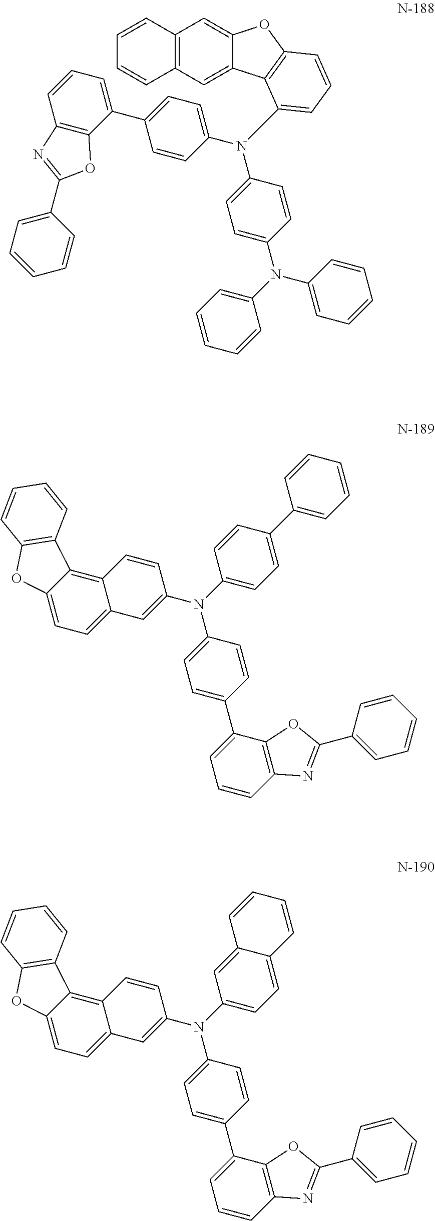 [SMILES: c1ccc(-c2nc3cccc(-c4ccc(N(c5ccc(N(c6ccccc6)c6ccccc6)cc5)c5cccc6oc7cc8ccccc8cc7c56)cc4)c3o2)cc1 ; c1ccc(-c2ccc(N(c3ccc(-c4cccc5nc(-c6ccccc6)oc45)cc3)c3ccc4c(ccc5oc6ccccc6c54)c3)cc2)cc1 ; c1ccc(-c2nc3cccc(-c4ccc(N(c5ccc6ccccc6c5)c5ccc6c(ccc7oc8ccccc8c76)c5)cc4)c3o2)cc1]

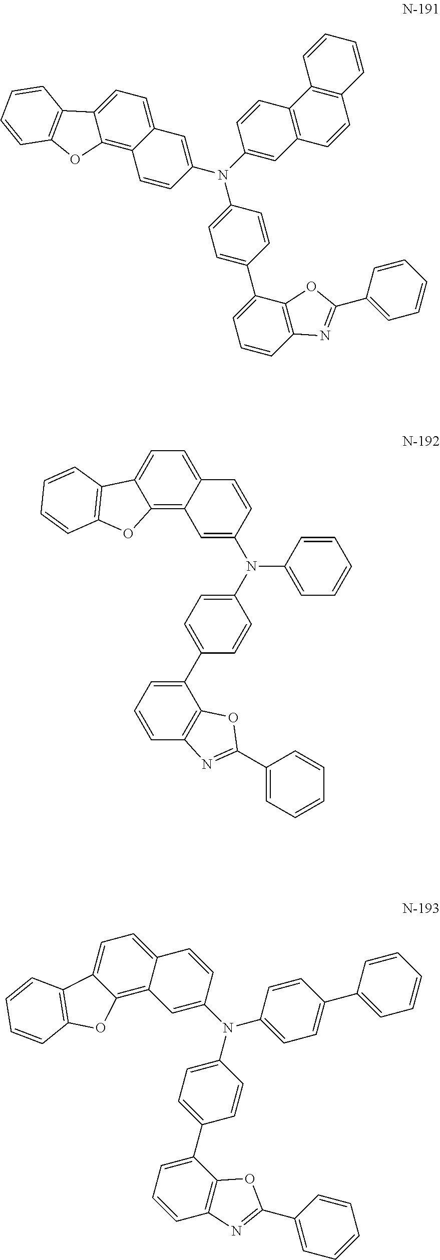 [SMILES: c1ccc(-c2nc3cccc(-c4ccc(N(c5ccc6c(ccc7ccccc76)c5)c5ccc6c(ccc7c8ccccc8oc67)c5)cc4)c3o2)cc1 ; c1ccc(-c2nc3cccc(-c4ccc(N(c5ccccc5)c5ccc6ccc7c8ccccc8oc7c6c5)cc4)c3o2)cc1 ; c1ccc(-c2ccc(N(c3ccc(-c4cccc5nc(-c6ccccc6)oc45)cc3)c3ccc4ccc5c6ccccc6oc5c4c3)cc2)cc1]

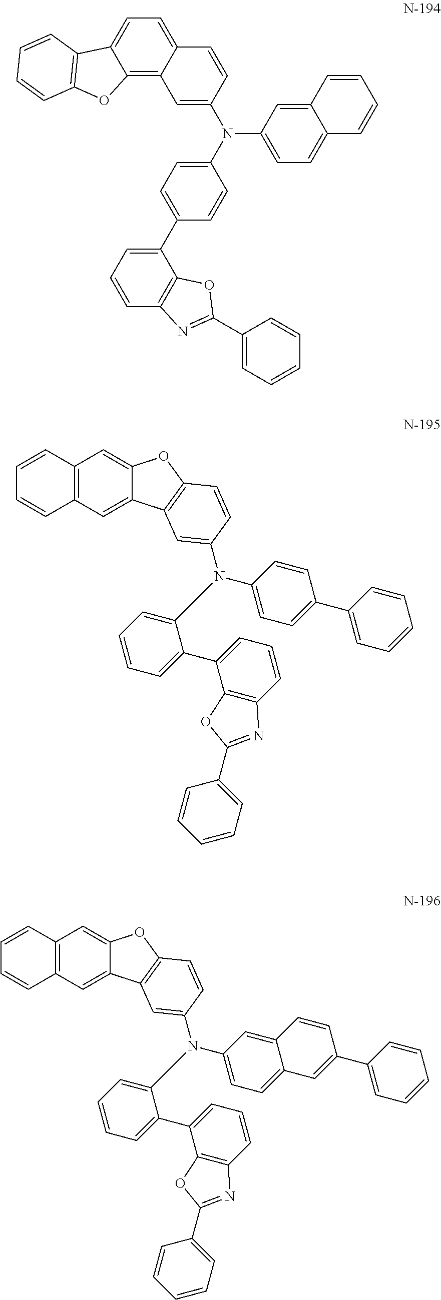 [SMILES: c1ccc(-c2nc3cccc(-c4ccc(N(c5ccc6ccccc6c5)c5ccc6ccc7c8ccccc8oc7c6c5)cc4)c3o2)cc1 ; c1ccc(-c2ccc(N(c3ccc4oc5cc6ccccc6cc5c4c3)c3ccccc3-c3cccc4nc(-c5ccccc5)oc34)cc2)cc1 ; c1ccc(-c2ccc3cc(N(c4ccc5oc6cc7ccccc7cc6c5c4)c4ccccc4-c4cccc5nc(-c6ccccc6)oc45)ccc3c2)cc1]

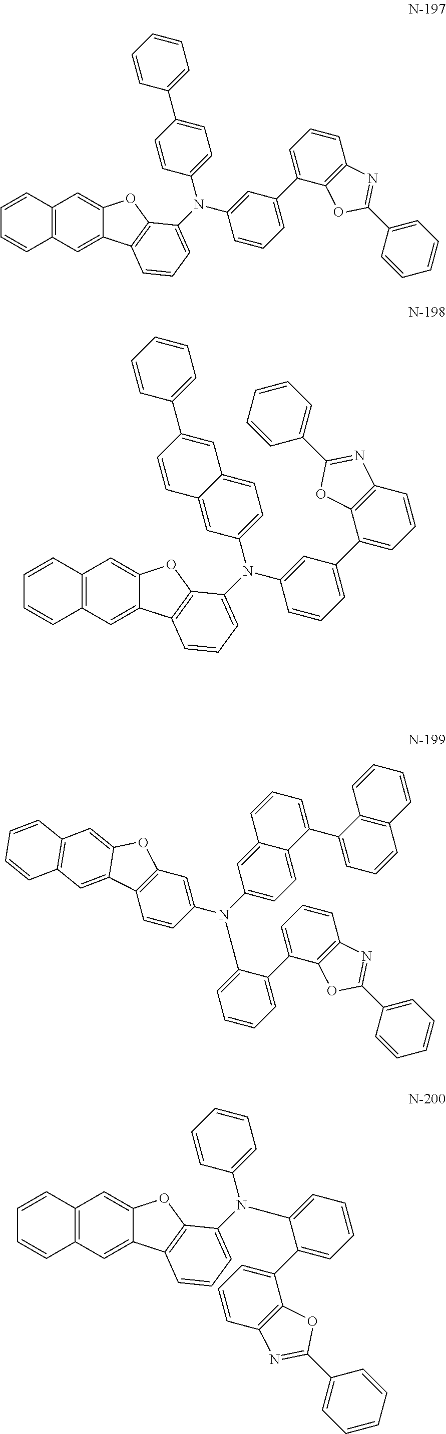 [SMILES: c1ccc(-c2ccc(N(c3cccc(-c4cccc5nc(-c6ccccc6)oc45)c3)c3cccc4c3oc3cc5ccccc5cc34)cc2)cc1 ; c1ccc(-c2ccc3cc(N(c4cccc(-c5cccc6nc(-c7ccccc7)oc56)c4)c4cccc5c4oc4cc6ccccc6cc45)ccc3c2)cc1 ; c1ccc(-c2nc3cccc(-c4ccccc4N(c4ccc5c(-c6cccc7ccccc67)cccc5c4)c4ccc5c(c4)oc4cc6ccccc6cc45)c3o2)cc1 ; c1ccc(-c2nc3cccc(-c4ccccc4N(c4ccccc4)c4cccc5c4oc4cc6ccccc6cc45)c3o2)cc1]

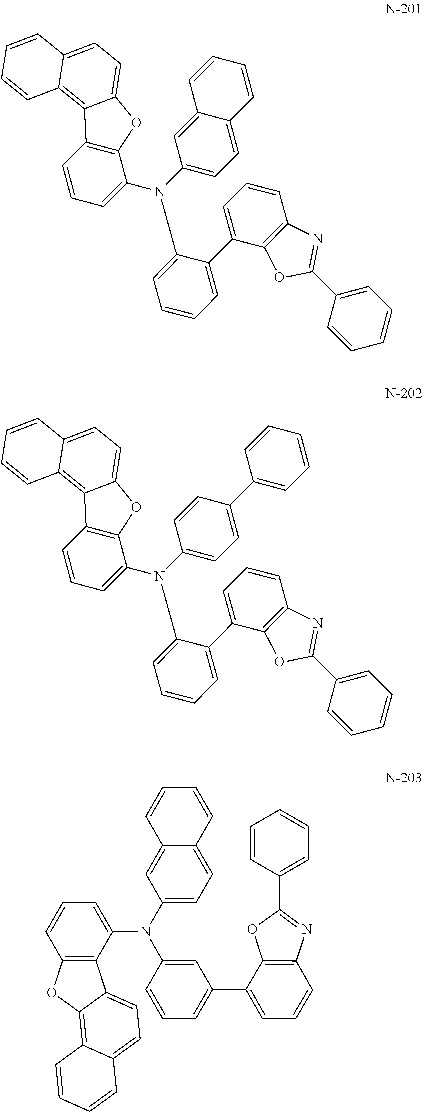 [SMILES: c1ccc(-c2nc3cccc(-c4ccccc4N(c4ccc5ccccc5c4)c4cccc5c4oc4ccc6ccccc6c45)c3o2)cc1 ; c1ccc(-c2ccc(N(c3ccccc3-c3cccc4nc(-c5ccccc5)oc34)c3cccc4c3oc3ccc5ccccc5c34)cc2)cc1 ; c1ccc(-c2nc3cccc(-c4cccc(N(c5ccc6ccccc6c5)c5cccc6oc7c8ccccc8ccc7c56)c4)c3o2)cc1]

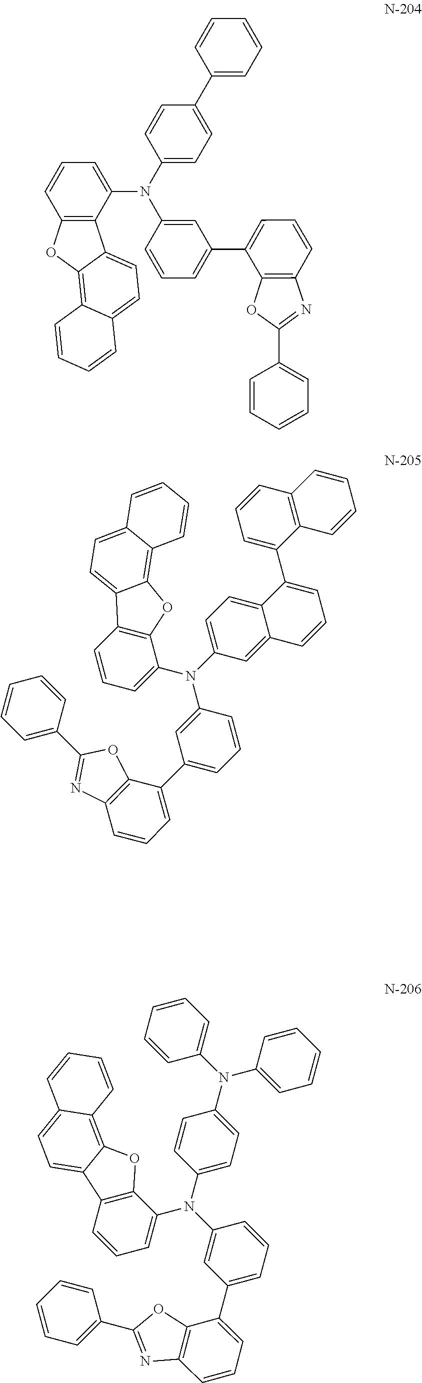 [SMILES: c1ccc(-c2ccc(N(c3cccc(-c4cccc5nc(-c6ccccc6)oc45)c3)c3cccc4oc5c6ccccc6ccc5c34)cc2)cc1 ; c1ccc(-c2nc3cccc(-c4cccc(N(c5ccc6c(-c7cccc8ccccc78)cccc6c5)c5cccc6c5oc5c7ccccc7ccc65)c4)c3o2)cc1 ; c1ccc(-c2nc3cccc(-c4cccc(N(c5ccc(N(c6ccccc6)c6ccccc6)cc5)c5cccc6c5oc5c7ccccc7ccc65)c4)c3o2)cc1]

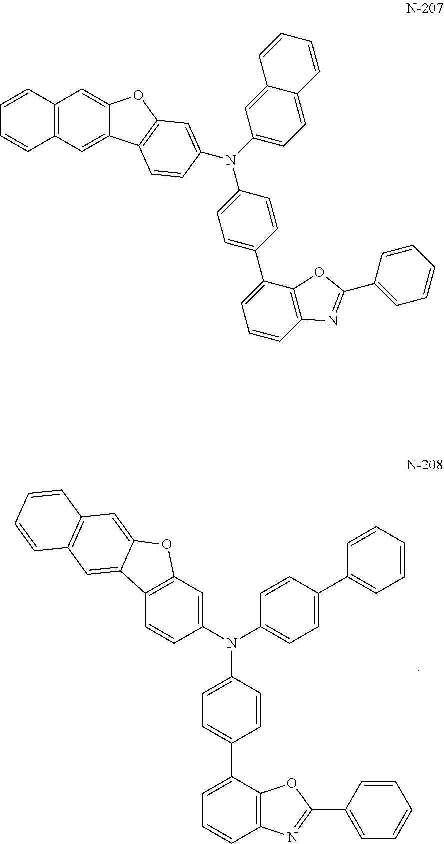 [SMILES: c1ccc(-c2nc3cccc(-c4ccc(N(c5ccc6ccccc6c5)c5ccc6c(c5)oc5cc7ccccc7cc56)cc4)c3o2)cc1 ; c1ccc(-c2ccc(N(c3ccc(-c4cccc5nc(-c6ccccc6)oc45)cc3)c3ccc4c(c3)oc3cc5ccccc5cc34)cc2)cc1]

18. The organic material composition according to claim 14, wherein in the Formula (2), X¹-X¹⁴ are all selected from CR, and R is defined as in claim 14.

19. The organic material composition according to claim 14, wherein any one of X¹-X⁶ is selected from N, and the others are CR, and R is defined as in claim 14.

20. The organic material composition according to claim 14, wherein any one of X¹-X⁶ is selected from N, and the others are CR; any one of X⁷-X¹⁴ is selected from N, and the others are CR, and R is defined as in claim 14.

21. The organic material composition according to claim 14, wherein Ar1 and Ar2 are each independently selected from the group consisting of unsubstituted C6-C15 aryl, unsubstituted C3-C20 heteroaryl.

22. The organic material composition according to claim 14, wherein Ar1 and Ar2 are each independently selected from unsubstituted A group;
the A group includes phenyl, naphthyl, biphenyl, terphenyl, phenanthryl, fluoranthenyl, triphenylene, fluorenyl, phenylfluorenyl, spirobifluorenyl, benzofuranyl, dibenzofuranyl, naphthobenzofuranyl, dinaphthofuranyl, benzothienyl, dibenzothienyl, naphthobenzothienyl, carbazolyl, phenylcarbazolyl, benzophenylcarbazolyl, and phenanthrenylbenzofuranyl.

23. The organic material composition according to claim 14, wherein Ar1 and Ar2 are each independently selected from the group consisting of phenyl, naphthyl, biphenyl, terphenyl, phenanthryl, fluoranthenyl, triphenylenyl, fluorenyl, phenylfluorenyl, spirobifluorenyl, dibenzofuranyl, naphthobenzofuranyl, dibenzothiophenyl, naphthobenzothiophenyl, carbazolyl, phenylcarbazolyl, benzocarbazolyl, and dibenzocarbazolyl.

24. The organic material composition according to claim 14, wherein the second compound has a structure as shown in any one of the following Formulas 2-1 to 2-28: 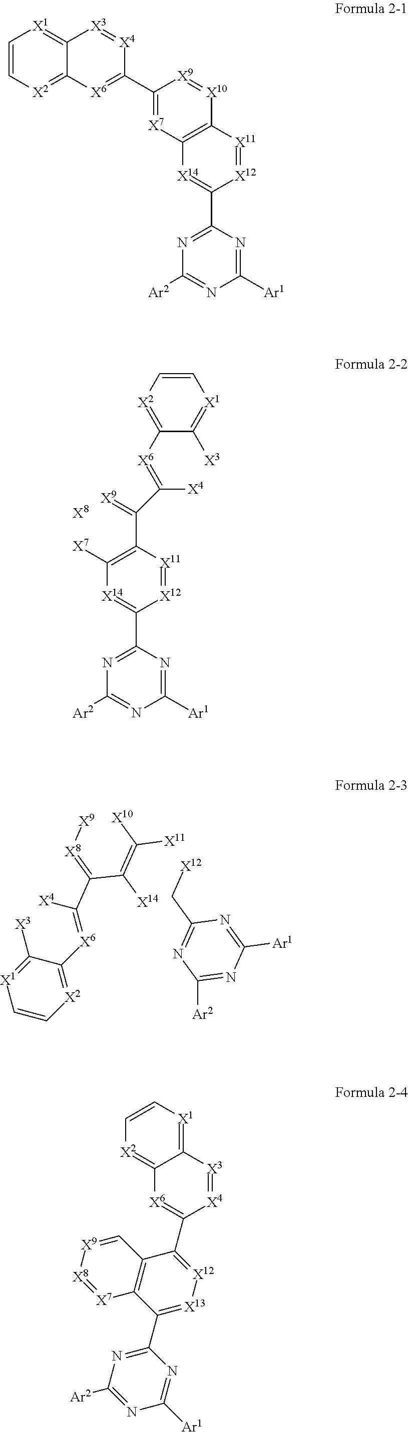 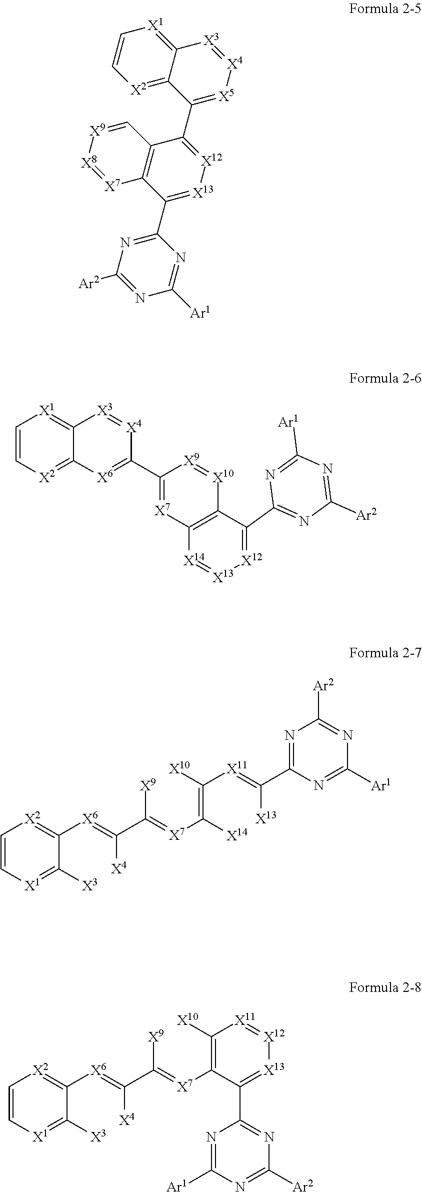 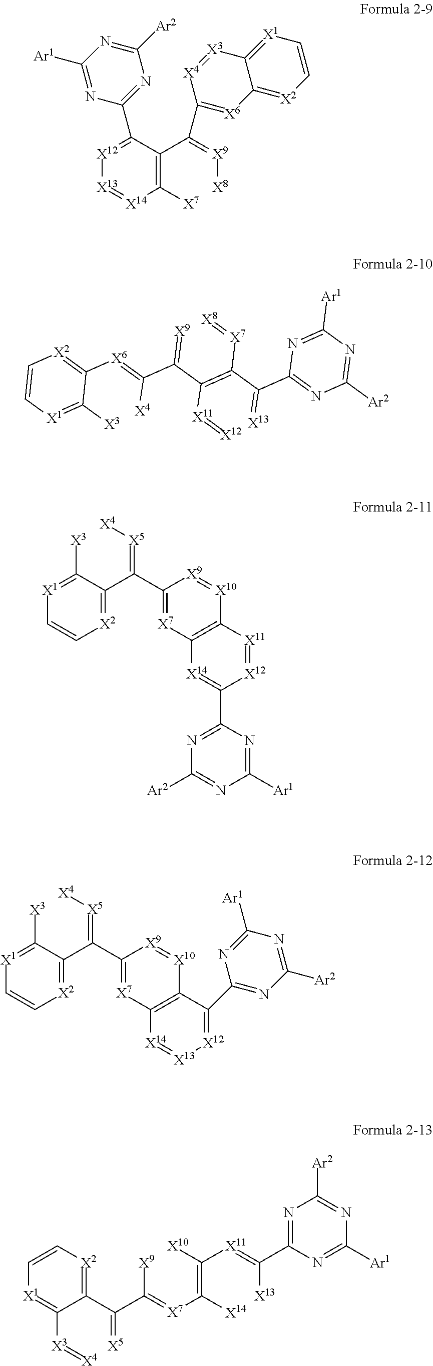 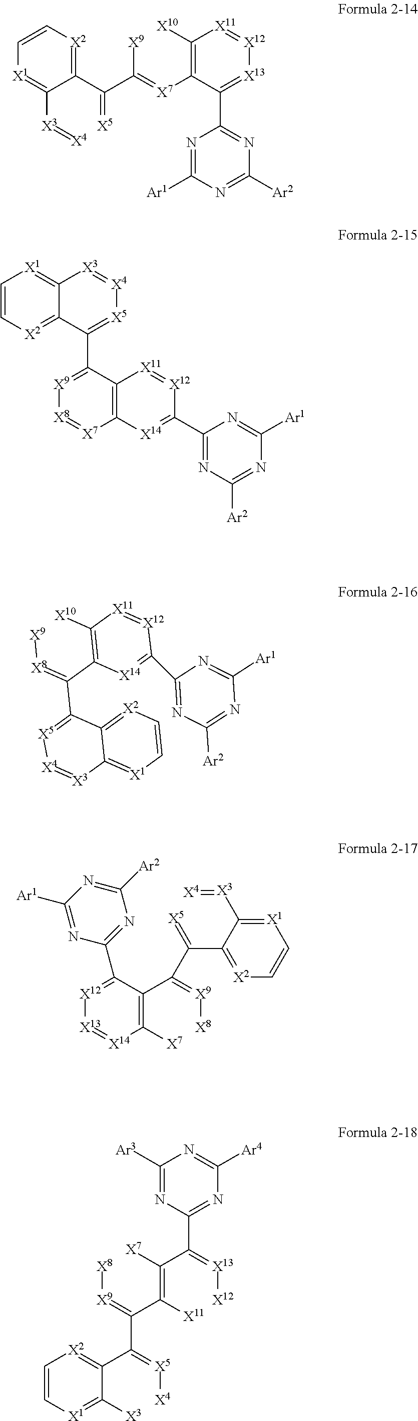 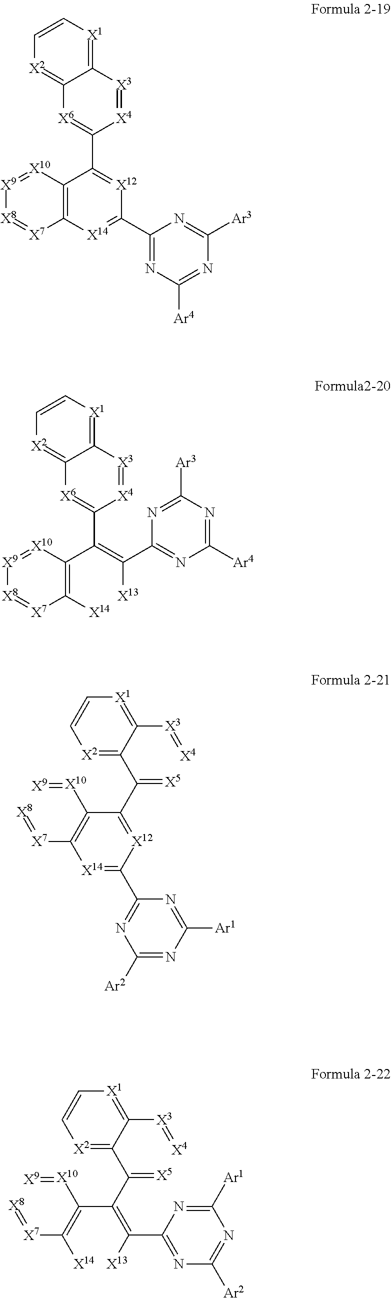 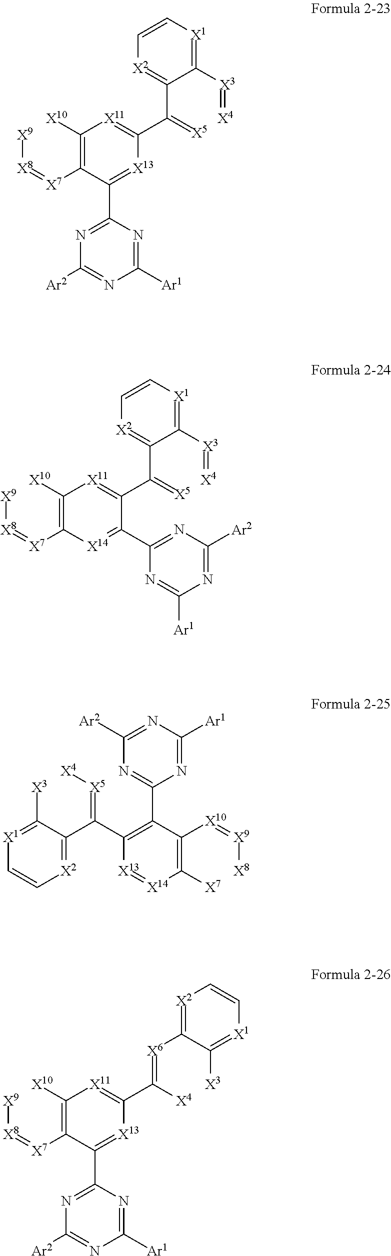 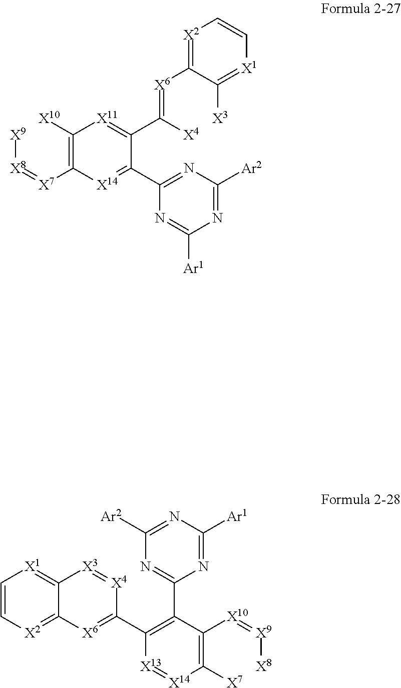
the definitions of X¹-X¹⁴, Ar1, and Ar2 are the same as those of claim 14.

25. The organic material composition according to claim 24, wherein the second compound has a structure shown in Formula 2-4 or Formula 2-5.

26. The organic material composition according to claim 24, wherein the second compound has any one structure of Formula 2-6, Formula 2-22, Formula 2-25, Formula 2-26, Formula 2-27, Formula 2-28, Formula 2-8 to Formula 2-20.

27. The organic material composition according to claim 14, wherein the second compound has a structure shown in any one of the following:

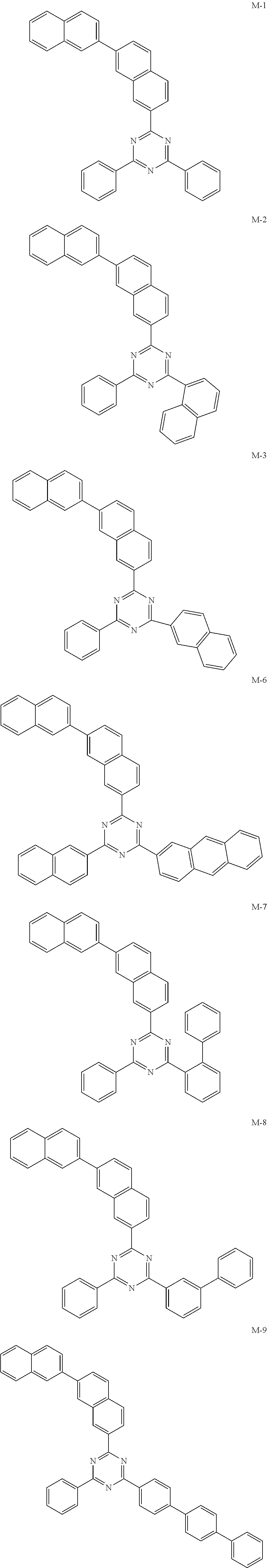

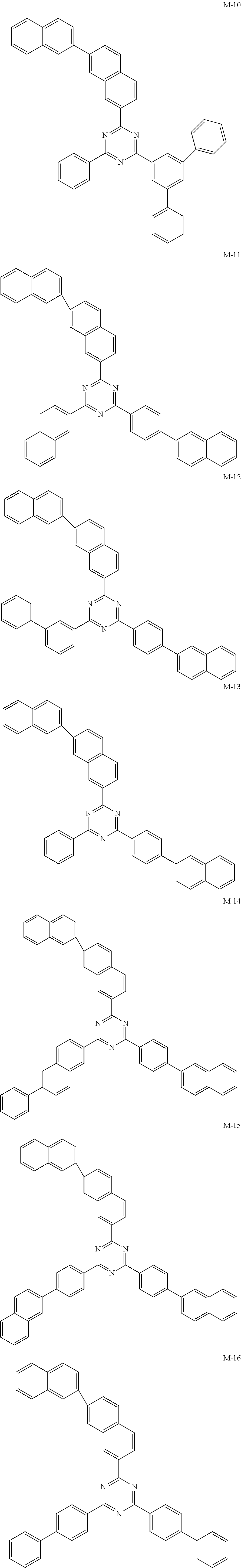

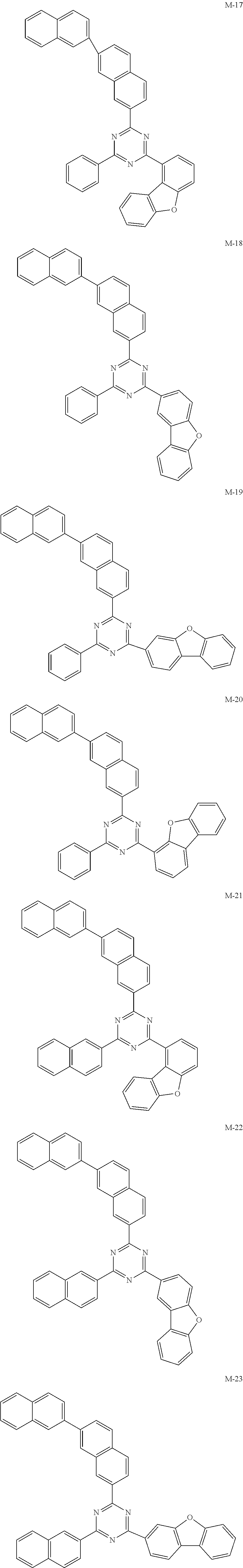

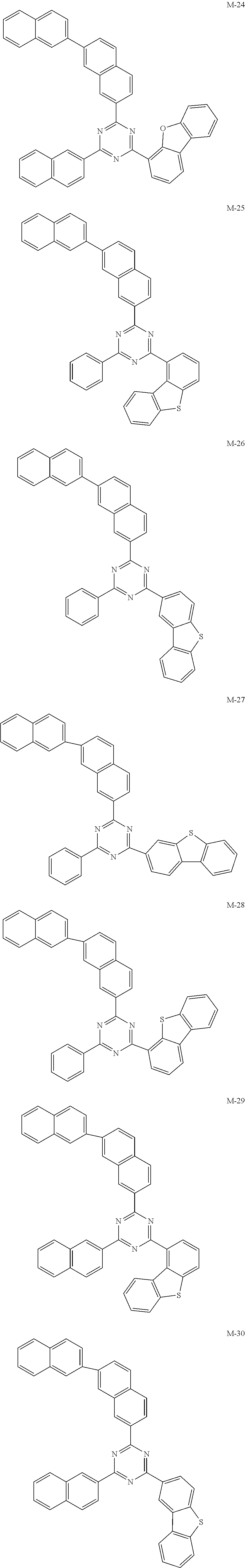

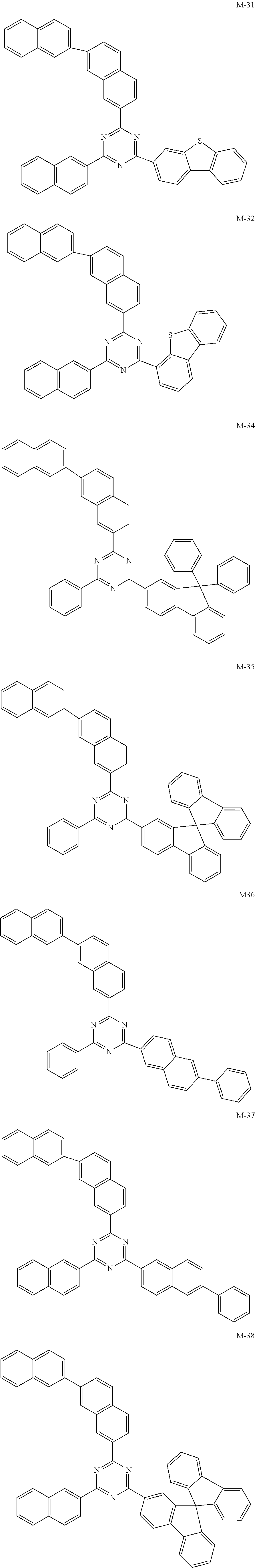

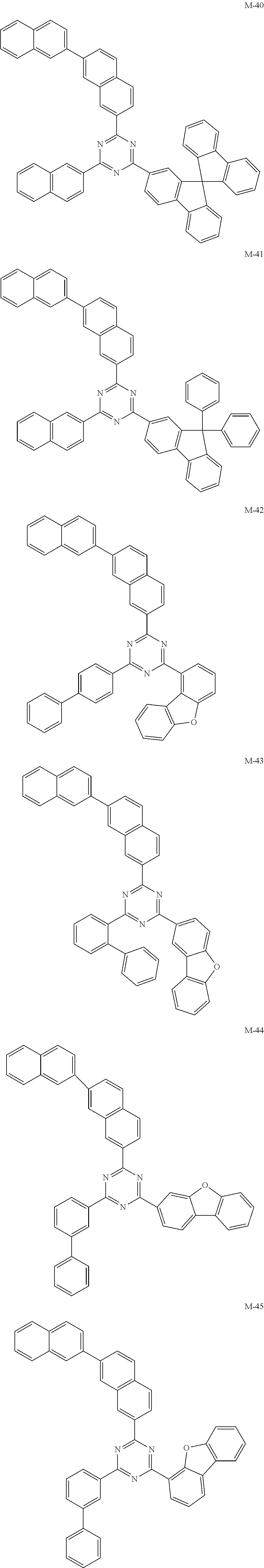

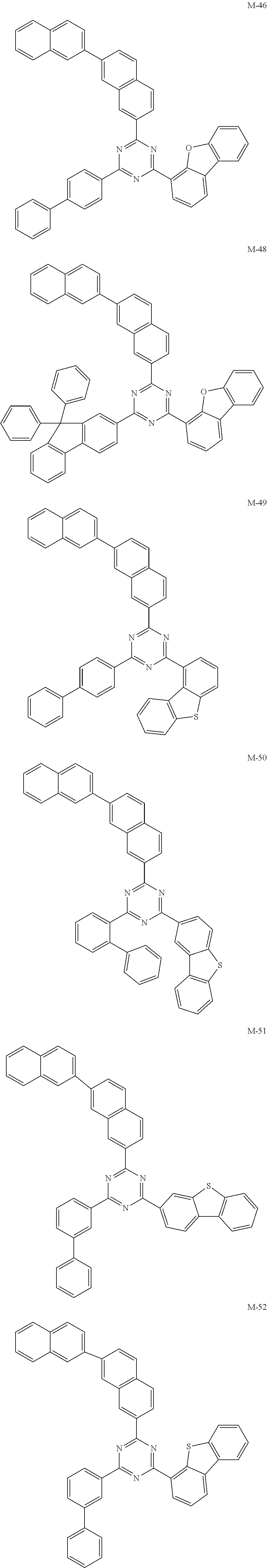

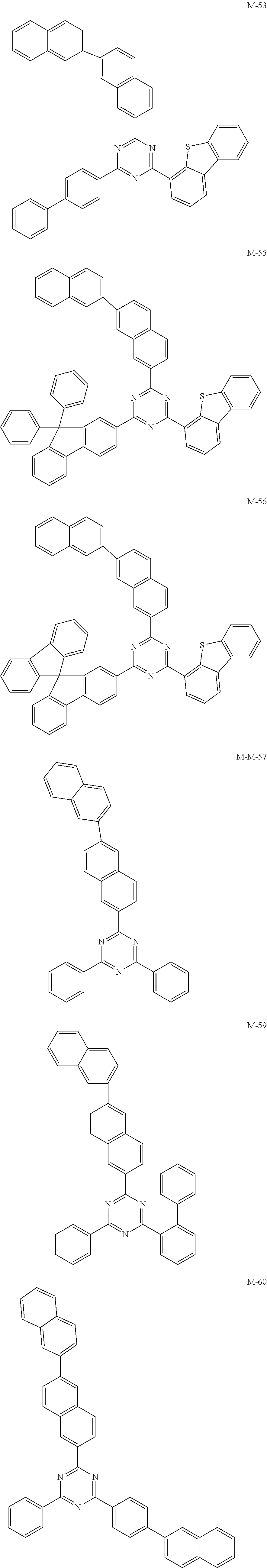

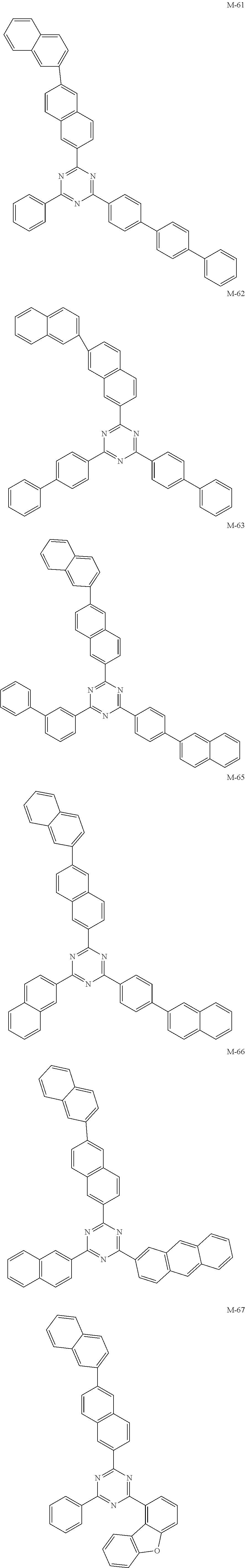

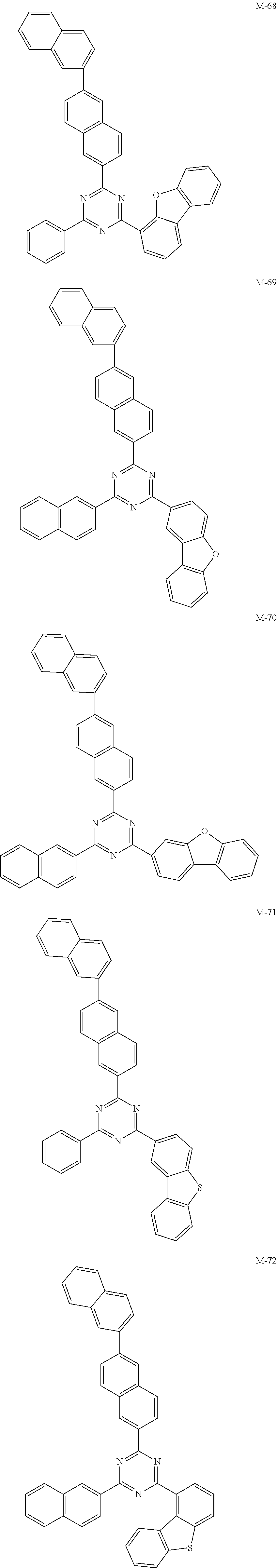

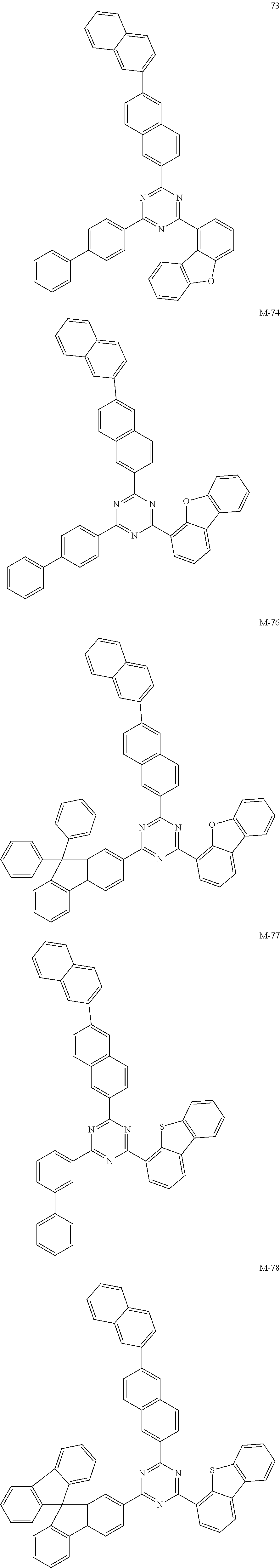

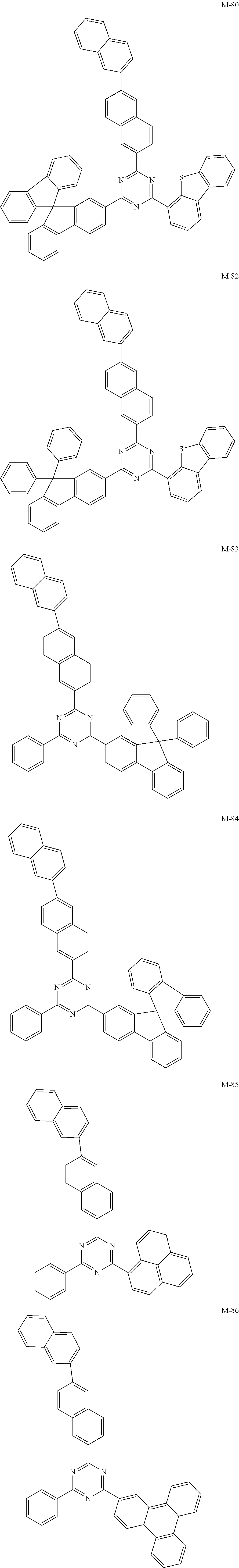

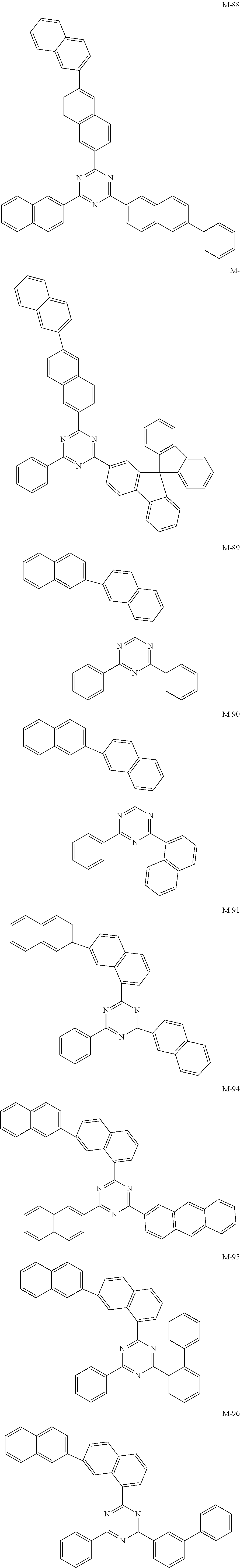

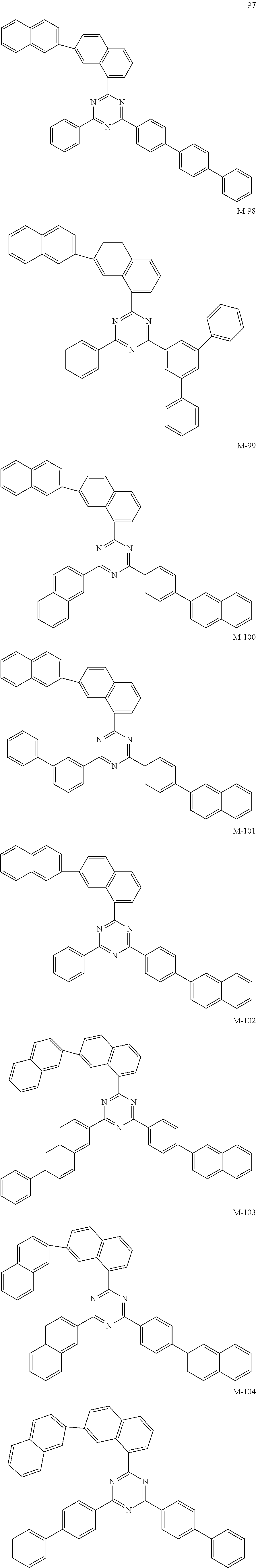

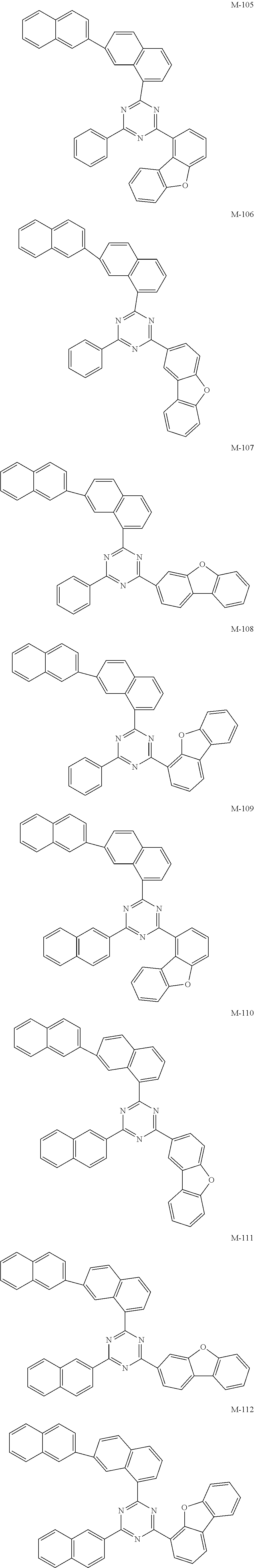

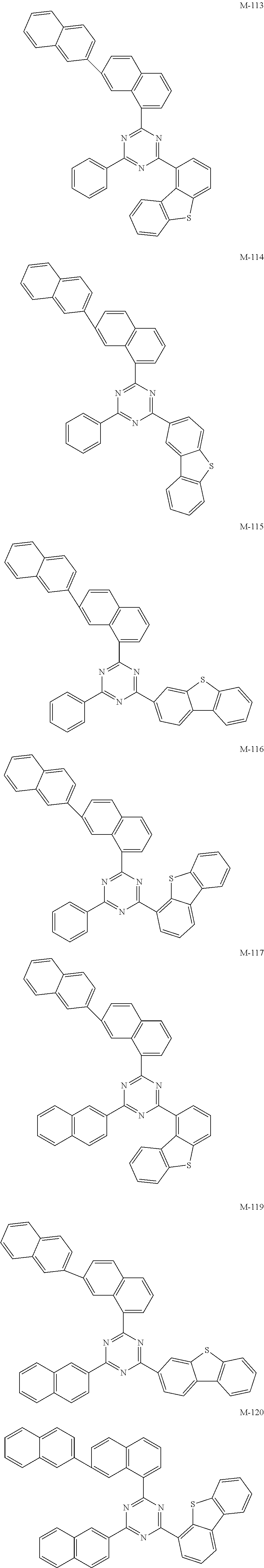

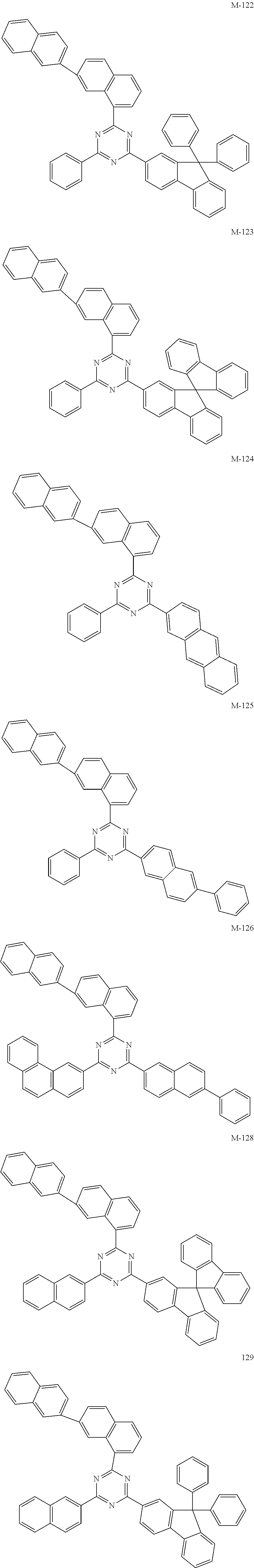

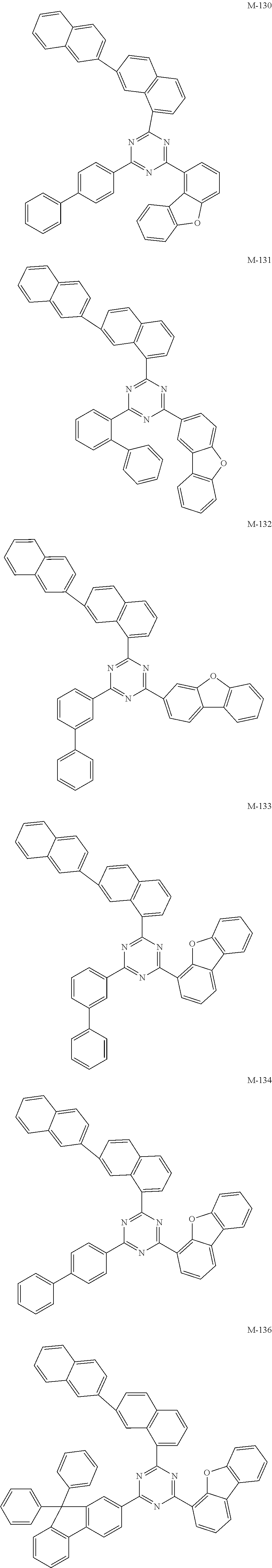

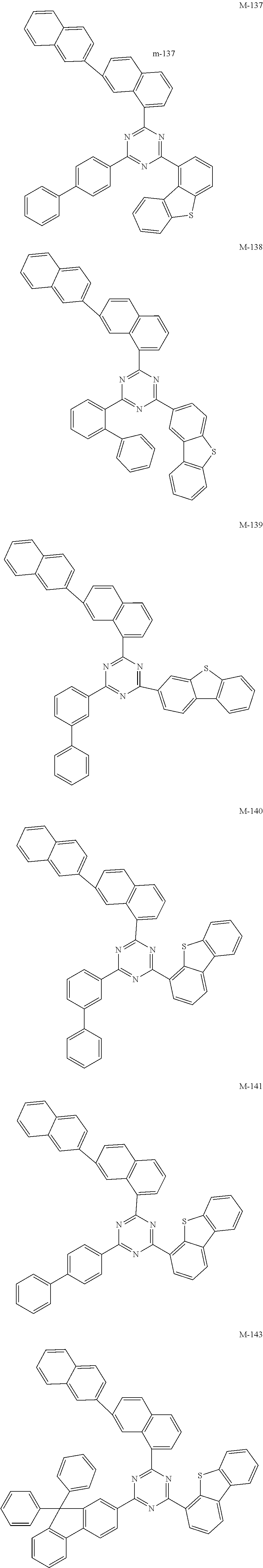

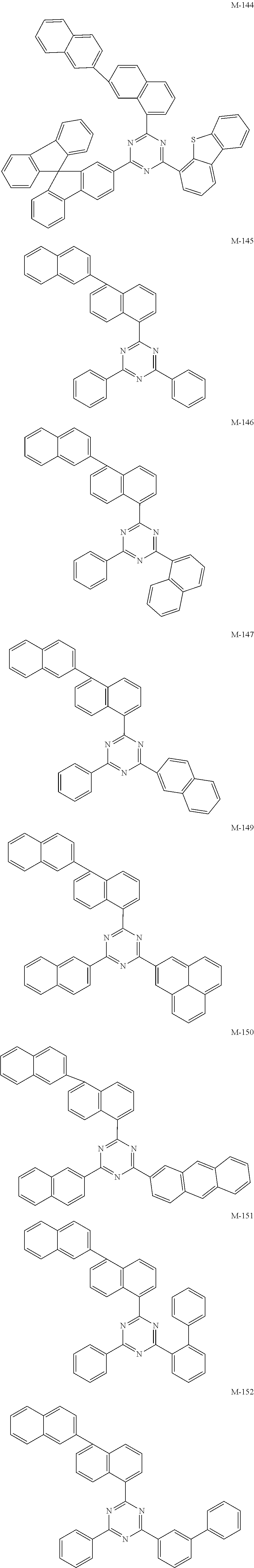

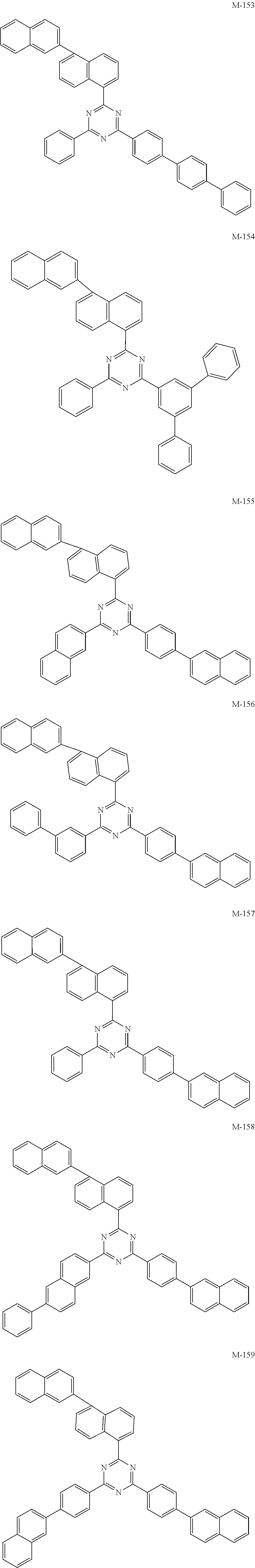

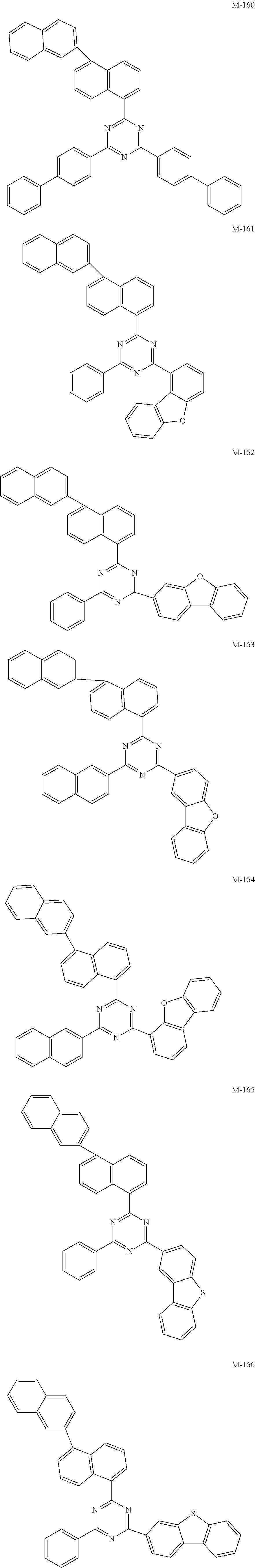

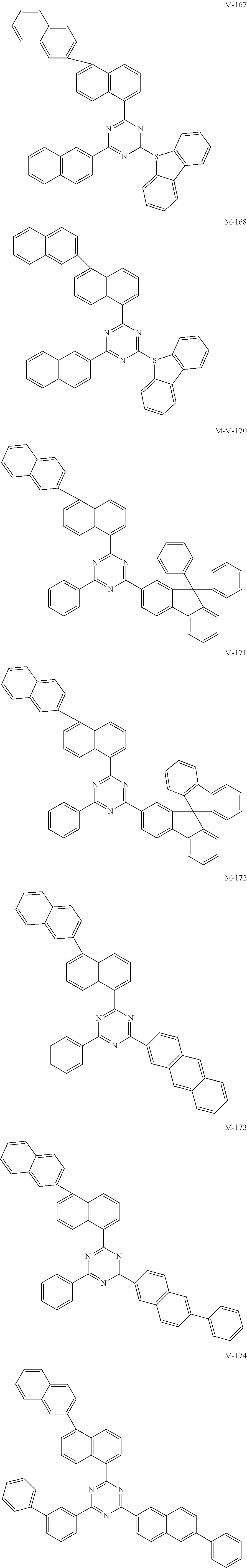

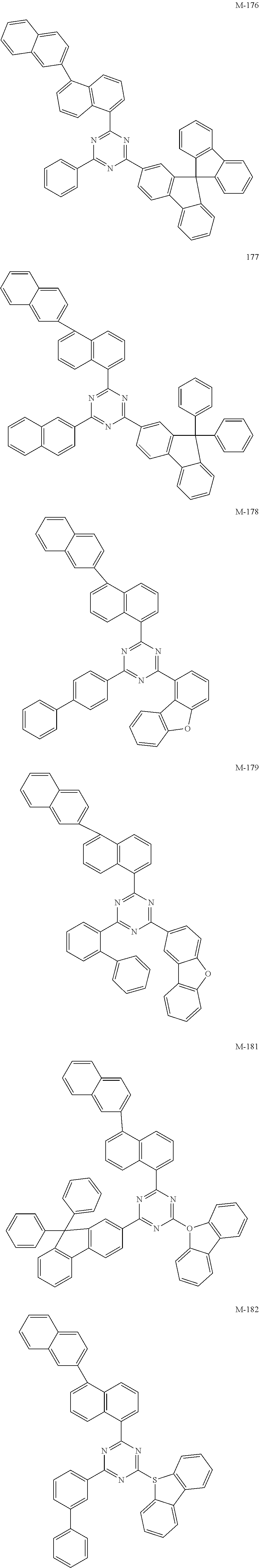

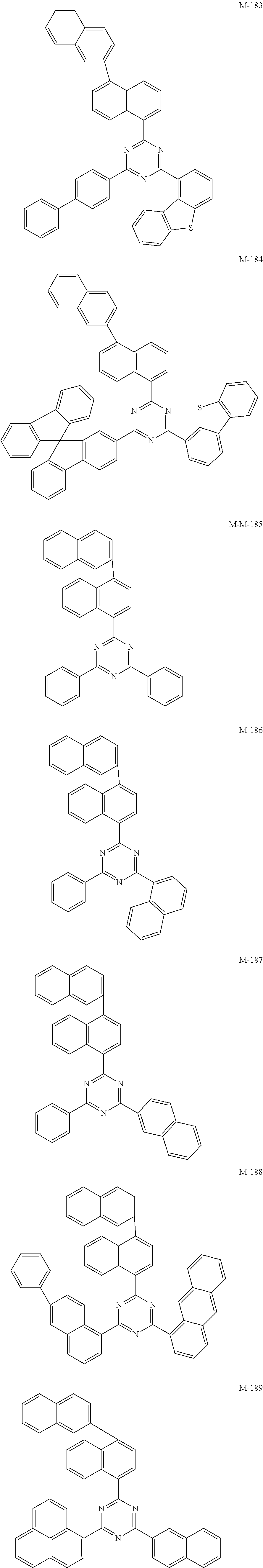

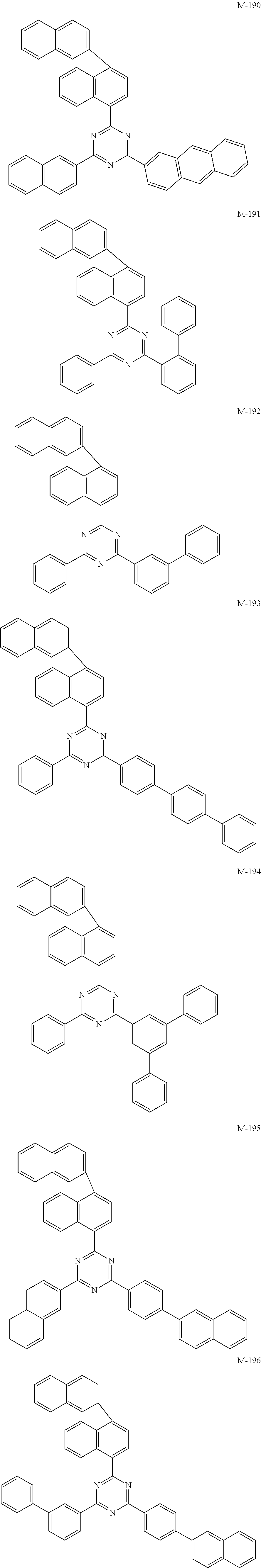

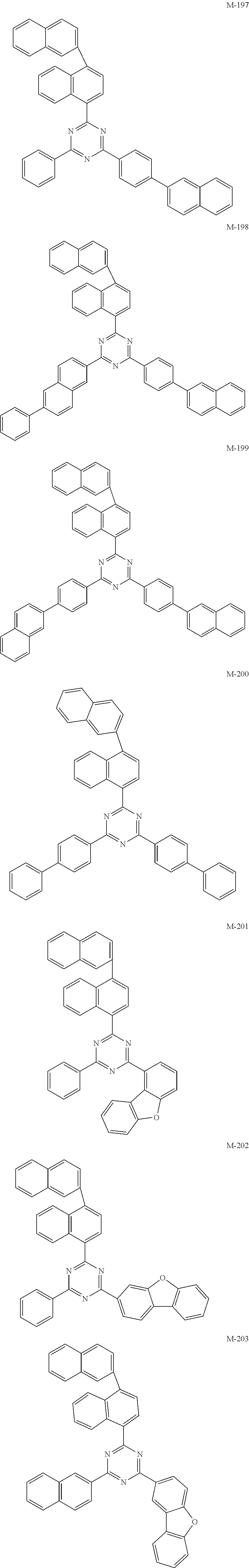

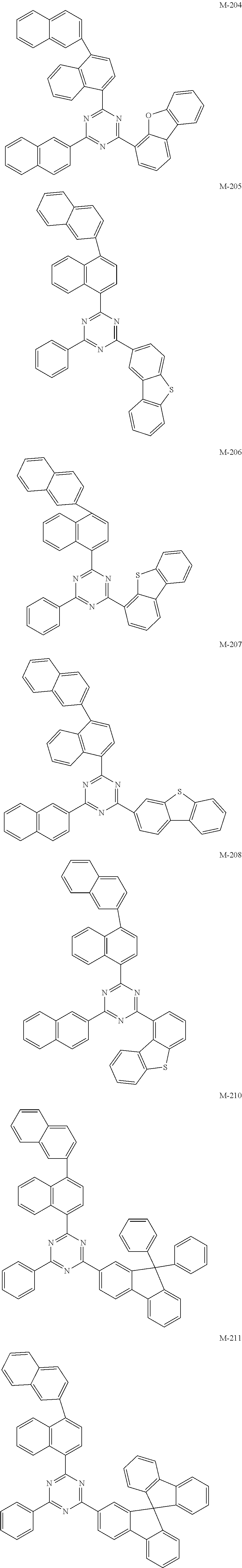

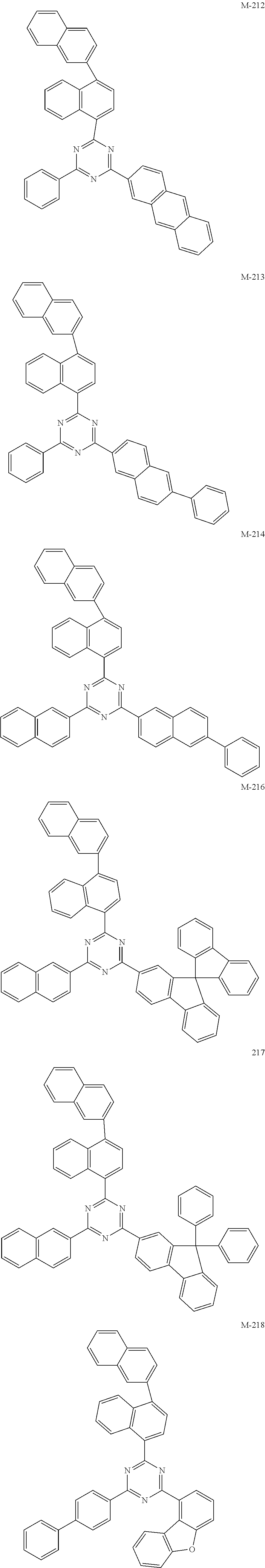

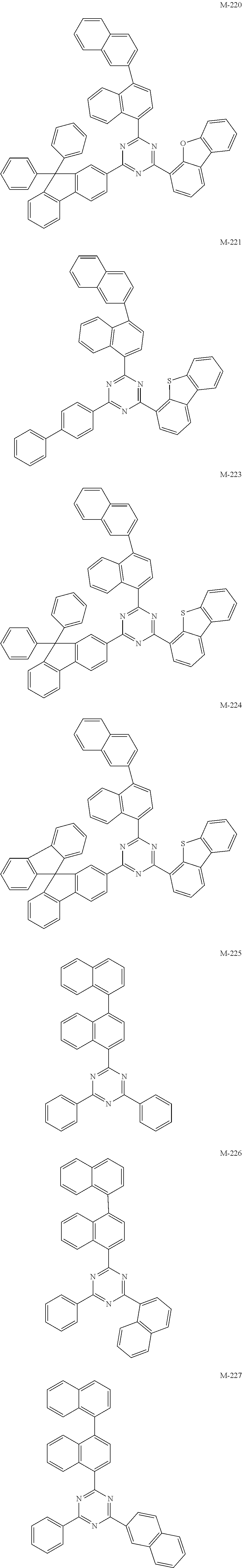

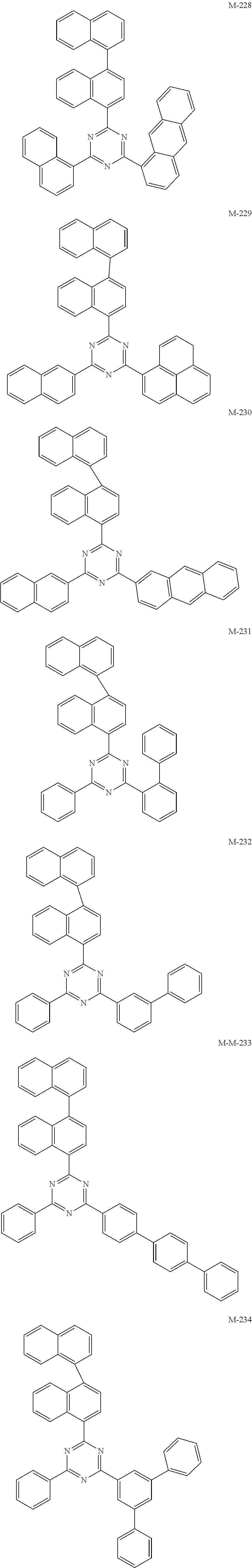

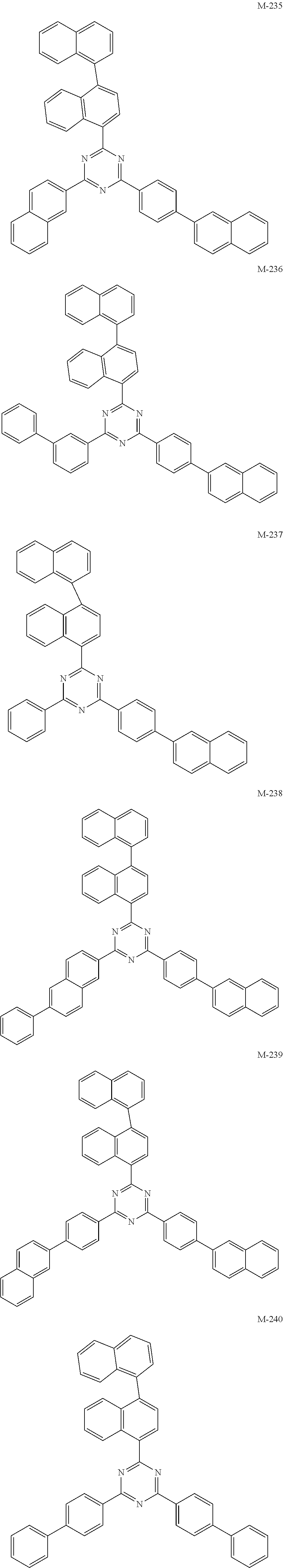

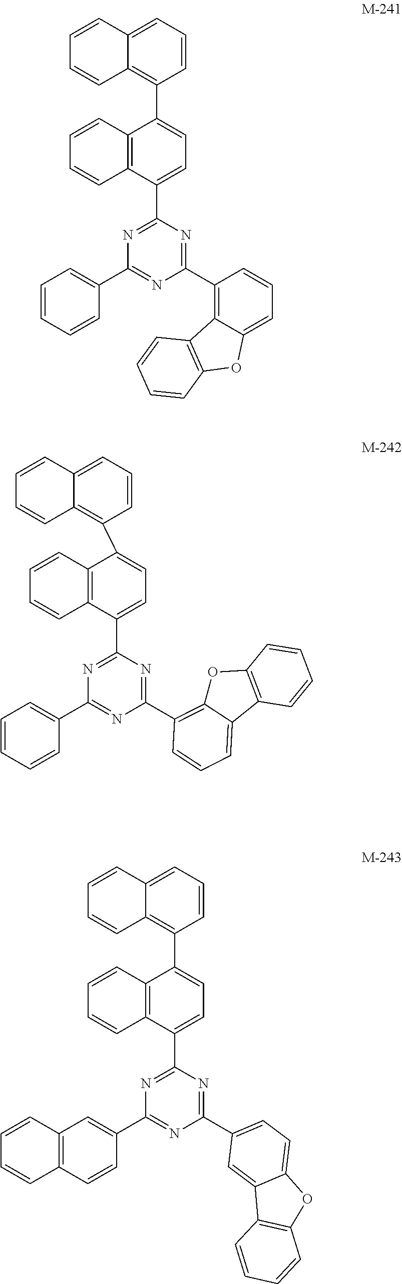 [SMILES: c1ccc(-c2nc(-c3ccc(-c4cccc5ccccc45)c4ccccc34)nc(-c3cccc4oc5ccccc5c34)n2)cc1 ; c1ccc(-c2nc(-c3ccc(-c4cccc5ccccc45)c4ccccc34)nc(-c3cccc4c3oc3ccccc34)n2)cc1 ; c1ccc2cc(-c3nc(-c4ccc5oc6ccccc6c5c4)nc(-c4ccc(-c5cccc6ccccc56)c5ccccc45)n3)ccc2c1]

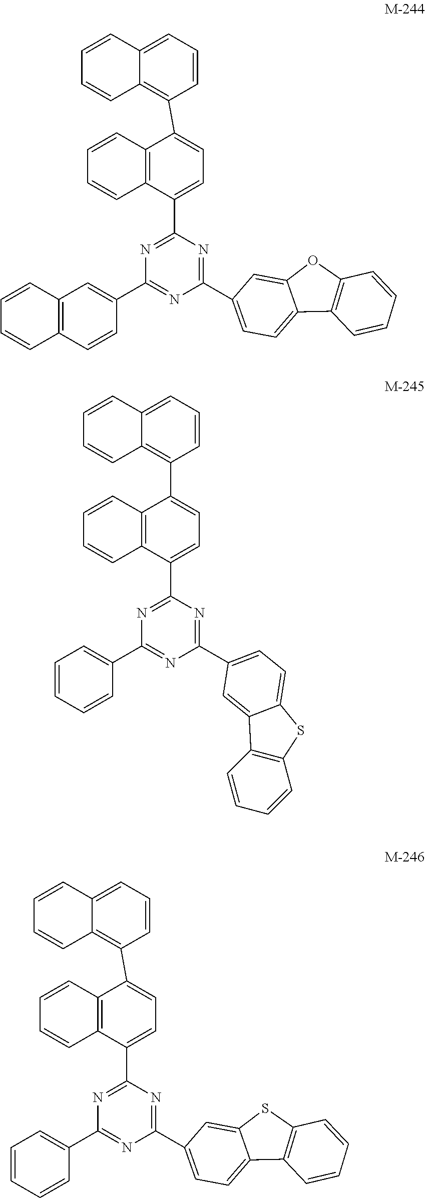 [SMILES: c1ccc2cc(-c3nc(-c4ccc5c(c4)oc4ccccc45)nc(-c4ccc(-c5cccc6ccccc56)c5ccccc45)n3)ccc2c1 ; c1ccc(-c2nc(-c3ccc4sc5ccccc5c4c3)nc(-c3ccc(-c4cccc5ccccc45)c4ccccc34)n2)cc1 ; c1ccc(-c2nc(-c3ccc4c(c3)sc3ccccc34)nc(-c3ccc(-c4cccc5ccccc45)c4ccccc34)n2)cc1]

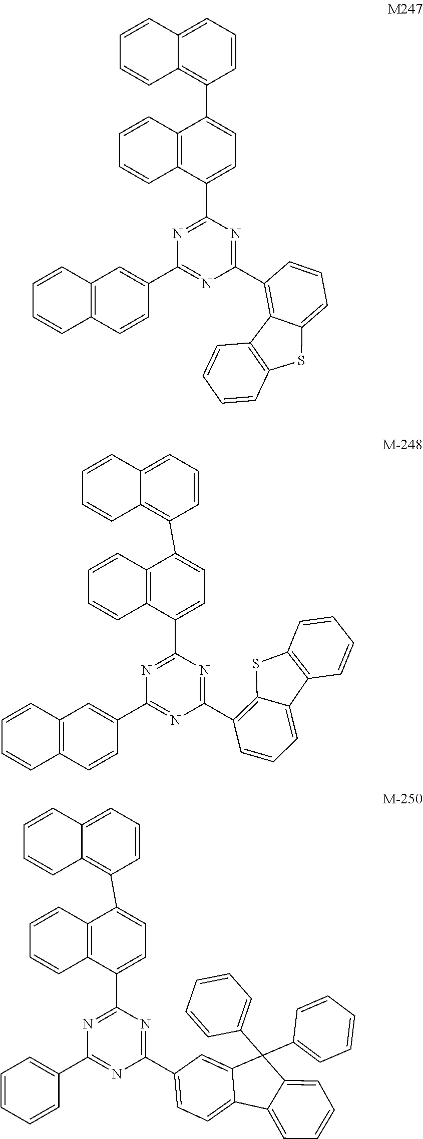

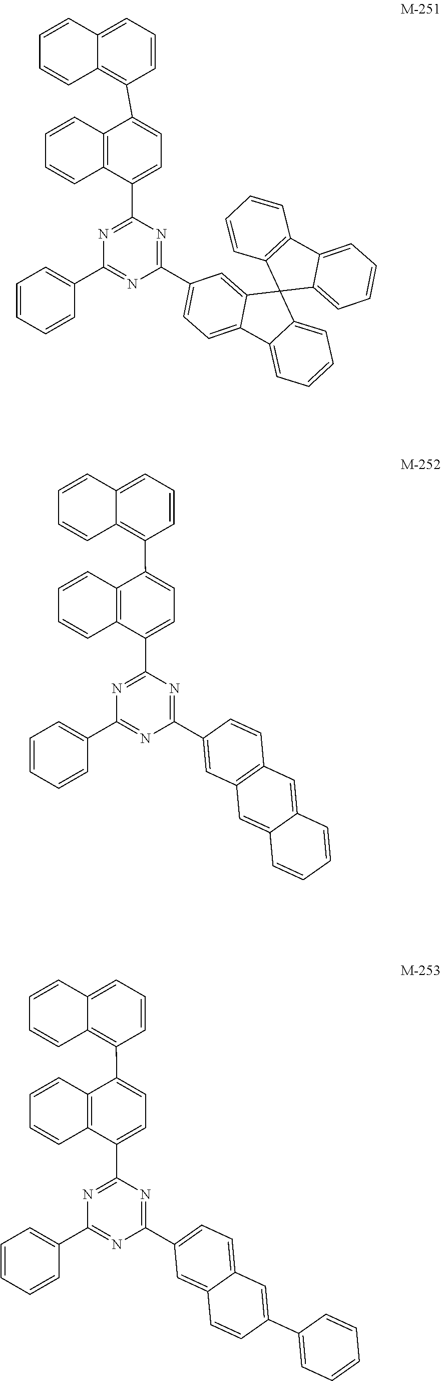 [SMILES: c1ccc(-c2nc(-c3ccc4c(c3)C3(c5ccccc5-c5ccccc53)c3ccccc3-4)nc(-c3ccc(-c4cccc5ccccc45)c4ccccc34)n2)cc1 ; c1ccc(-c2nc(-c3ccc4cc5ccccc5cc4c3)nc(-c3ccc(-c4cccc5ccccc45)c4ccccc34)n2)cc1 ; c1ccc(-c2ccc3cc(-c4nc(-c5ccccc5)nc(-c5ccc(-c6cccc7ccccc67)c6ccccc56)n4)ccc3c2)cc1]

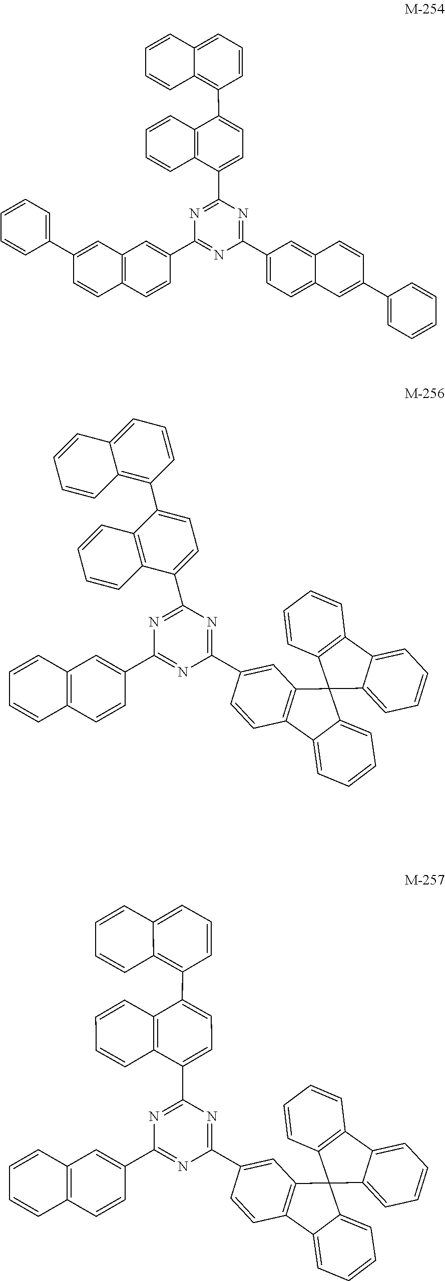 [SMILES: c1ccc(-c2ccc3cc(-c4nc(-c5ccc6ccc(-c7ccccc7)cc6c5)nc(-c5ccc(-c6cccc7ccccc67)c6ccccc56)n4)ccc3c2)cc1 ; c1ccc2c(c1)-c1ccccc1C21c2ccccc2-c2ccc(-c3nc(-c4ccc5ccccc5c4)nc(-c4ccc(-c5cccc6ccccc56)c5ccccc45)n3)cc21 ; c1ccc2c(c1)-c1ccccc1C21c2ccccc2-c2ccc(-c3nc(-c4ccc5ccccc5c4)nc(-c4ccc(-c5cccc6ccccc56)c5ccccc45)n3)cc21]

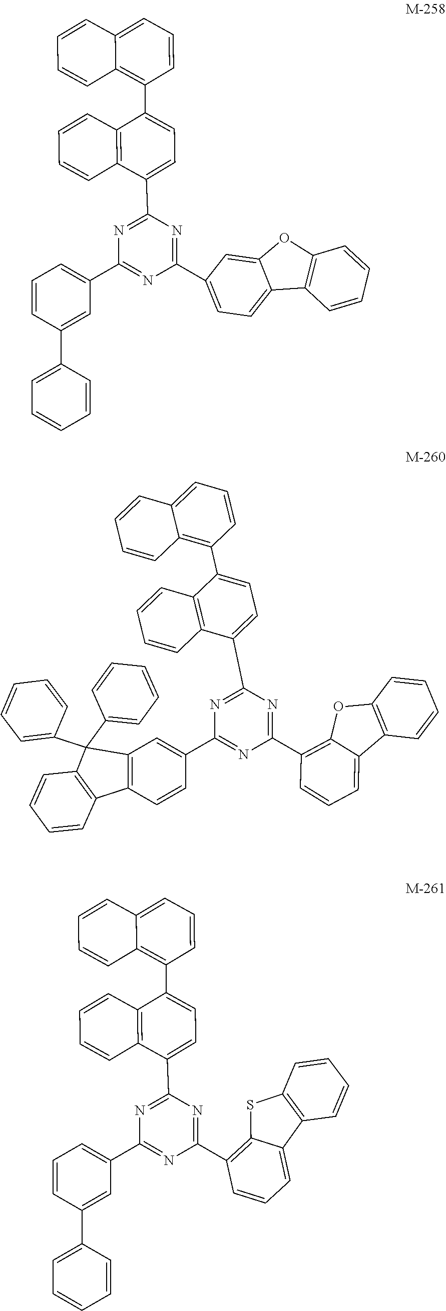 [SMILES: c1ccc(-c2cccc(-c3nc(-c4ccc5c(c4)oc4ccccc45)nc(-c4ccc(-c5cccc6ccccc56)c5ccccc45)n3)c2)cc1 ; c1ccc(C2(c3ccccc3)c3ccccc3-c3ccc(-c4nc(-c5ccc(-c6cccc7ccccc67)c6ccccc56)nc(-c5cccc6c5oc5ccccc56)n4)cc32)cc1 ; c1ccc(-c2cccc(-c3nc(-c4ccc(-c5cccc6ccccc56)c5ccccc45)nc(-c4cccc5c4sc4ccccc45)n3)c2)cc1]

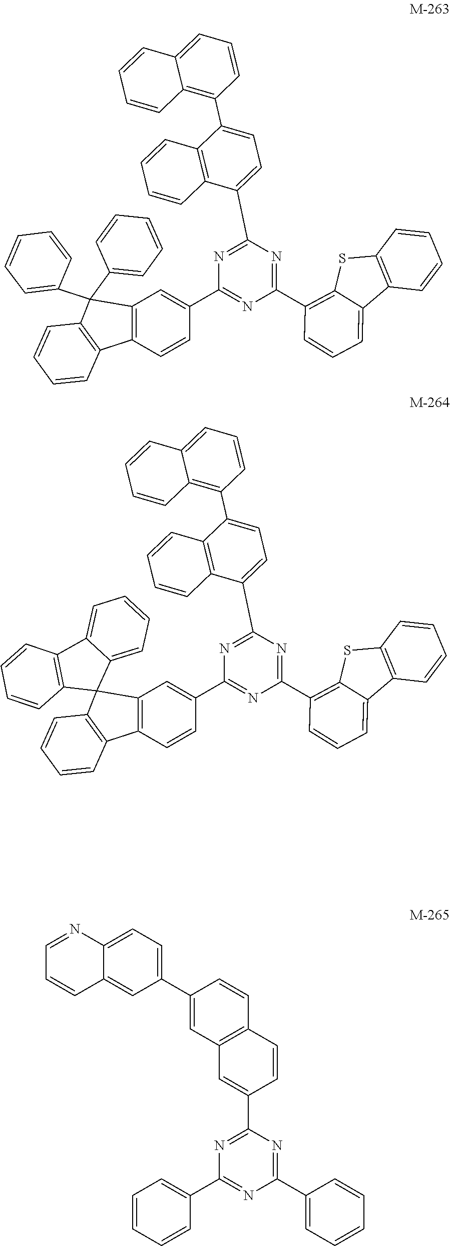 [SMILES: c1ccc(C2(c3ccccc3)c3ccccc3-c3ccc(-c4nc(-c5ccc(-c6cccc7ccccc67)c6ccccc56)nc(-c5cccc6c5sc5ccccc56)n4)cc32)cc1 ; c1ccc2c(c1)-c1ccccc1C21c2ccccc2-c2ccc(-c3nc(-c4ccc(-c5cccc6ccccc56)c5ccccc45)nc(-c4cccc5c4sc4ccccc45)n3)cc21 ; c1ccc(-c2nc(-c3ccccc3)nc(-c3ccc4ccc(-c5ccc6ncccc6c5)cc4c3)n2)cc1]

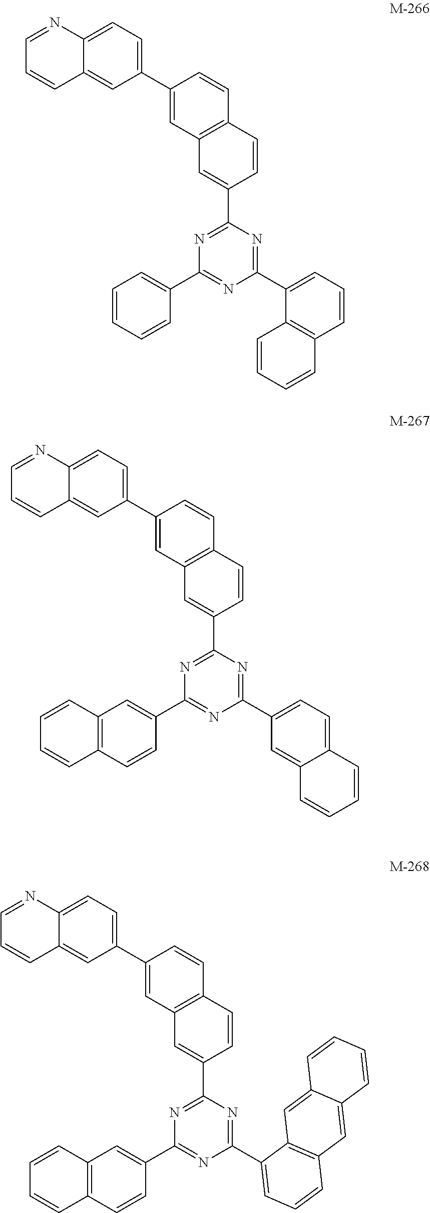 [SMILES: c1ccc(-c2nc(-c3ccc4ccc(-c5ccc6ncccc6c5)cc4c3)nc(-c3cccc4ccccc34)n2)cc1 ; c1ccc2cc(-c3nc(-c4ccc5ccccc5c4)nc(-c4ccc5ccc(-c6ccc7ncccc7c6)cc5c4)n3)ccc2c1 ; c1ccc2cc(-c3nc(-c4ccc5ccc(-c6ccc7ncccc7c6)cc5c4)nc(-c4cccc5cc6ccccc6cc45)n3)ccc2c1]

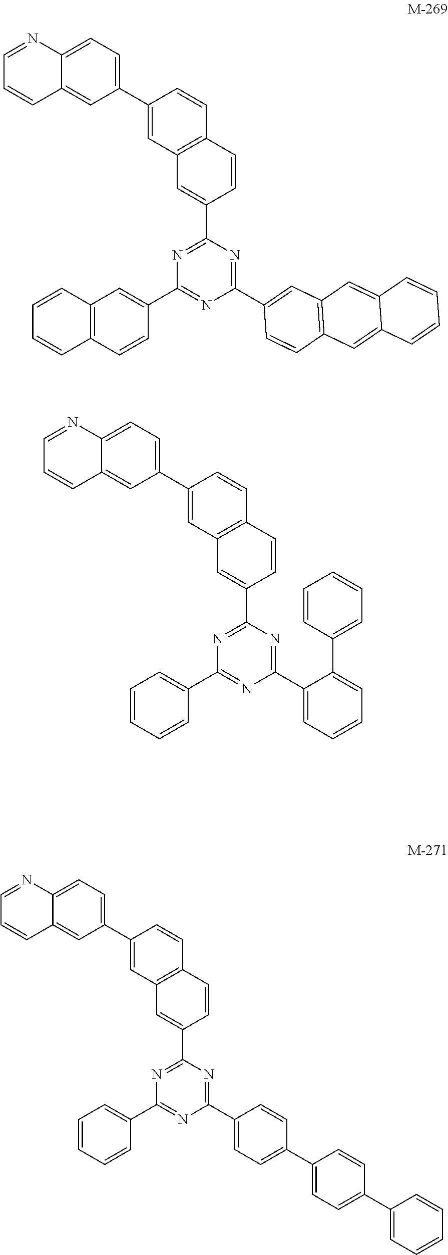 [SMILES: c1ccc2cc(-c3nc(-c4ccc5ccc(-c6ccc7ncccc7c6)cc5c4)nc(-c4ccc5cc6ccccc6cc5c4)n3)ccc2c1 ; c1ccc(-c2nc(-c3ccc4ccc(-c5ccc6ncccc6c5)cc4c3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6c5)n4)cc3)cc2)cc1]

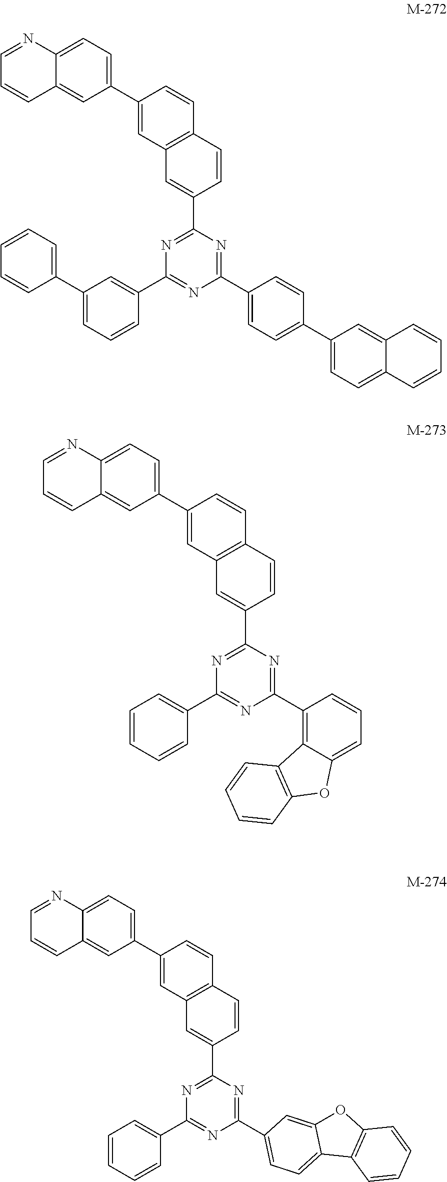 [SMILES: c1ccc(-c2cccc(-c3nc(-c4ccc(-c5ccc6ccccc6c5)cc4)nc(-c4ccc5ccc(-c6ccc7ncccc7c6)cc5c4)n3)c2)cc1 ; c1ccc(-c2nc(-c3ccc4ccc(-c5ccc6ncccc6c5)cc4c3)nc(-c3cccc4oc5ccccc5c34)n2)cc1 ; c1ccc(-c2nc(-c3ccc4ccc(-c5ccc6ncccc6c5)cc4c3)nc(-c3ccc4c(c3)oc3ccccc34)n2)cc1]

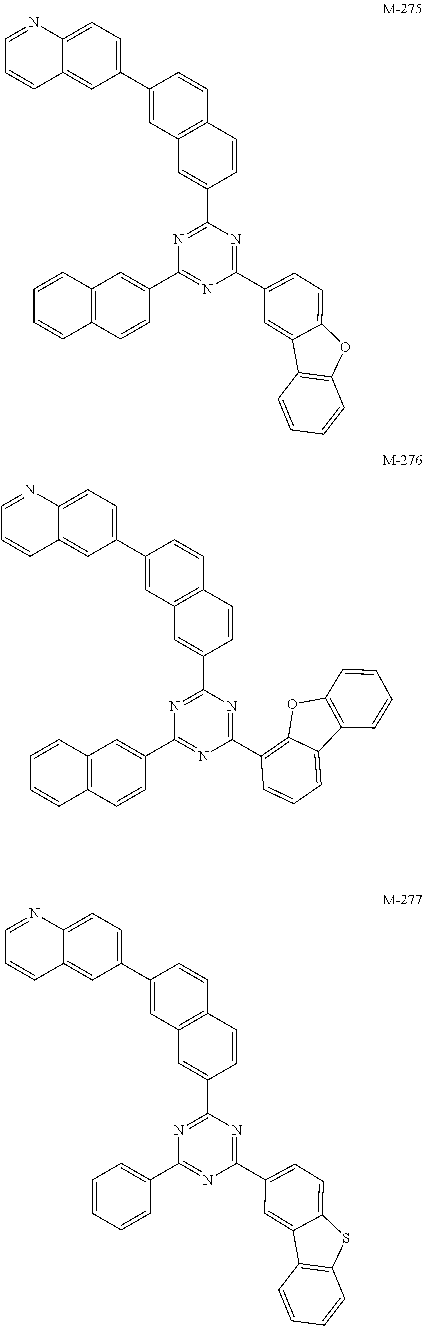 [SMILES: c1ccc2cc(-c3nc(-c4ccc5ccc(-c6ccc7ncccc7c6)cc5c4)nc(-c4ccc5oc6ccccc6c5c4)n3)ccc2c1 ; c1ccc2cc(-c3nc(-c4ccc5ccc(-c6ccc7ncccc7c6)cc5c4)nc(-c4cccc5c4oc4ccccc45)n3)ccc2c1 ; c1ccc(-c2nc(-c3ccc4ccc(-c5ccc6ncccc6c5)cc4c3)nc(-c3ccc4sc5ccccc5c4c3)n2)cc1]

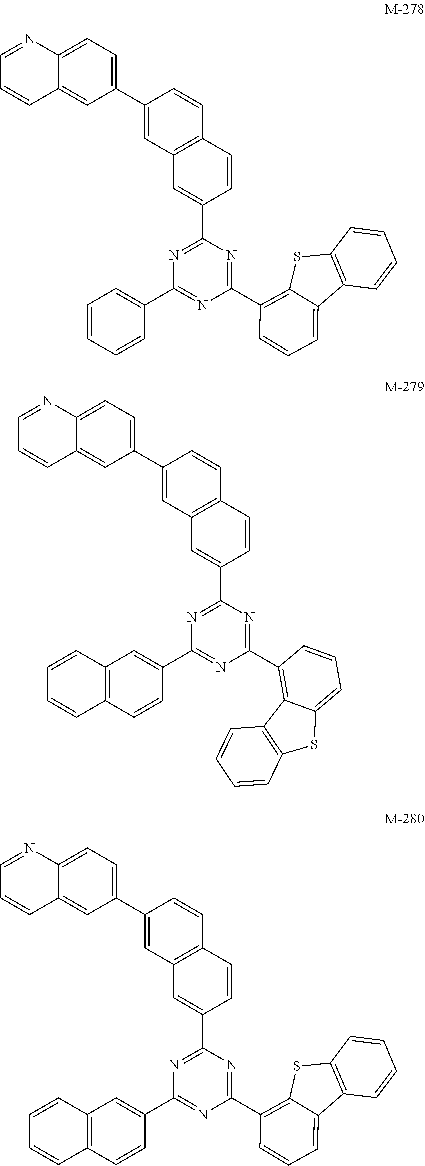 [SMILES: c1ccc(-c2nc(-c3ccc4ccc(-c5ccc6ncccc6c5)cc4c3)nc(-c3cccc4c3sc3ccccc34)n2)cc1 ; c1ccc2cc(-c3nc(-c4ccc5ccc(-c6ccc7ncccc7c6)cc5c4)nc(-c4cccc5sc6ccccc6c45)n3)ccc2c1 ; c1ccc2cc(-c3nc(-c4ccc5ccc(-c6ccc7ncccc7c6)cc5c4)nc(-c4cccc5c4sc4ccccc45)n3)ccc2c1]

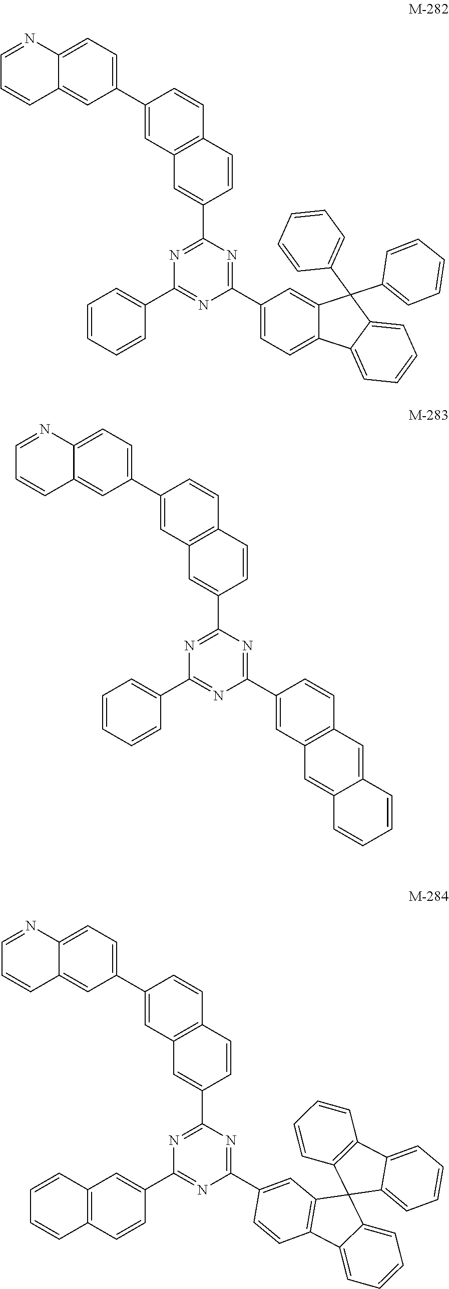 [SMILES: c1ccc(-c2nc(-c3ccc4c(c3)C(c3ccccc3)(c3ccccc3)c3ccccc3-4)nc(-c3ccc4ccc(-c5ccc6ncccc6c5)cc4c3)n2)cc1 ; c1ccc(-c2nc(-c3ccc4ccc(-c5ccc6ncccc6c5)cc4c3)nc(-c3ccc4cc5ccccc5cc4c3)n2)cc1 ; c1ccc2c(c1)-c1ccccc1C21c2ccccc2-c2ccc(-c3nc(-c4ccc5ccccc5c4)nc(-c4ccc5ccc(-c6ccc7ncccc7c6)cc5c4)n3)cc21]

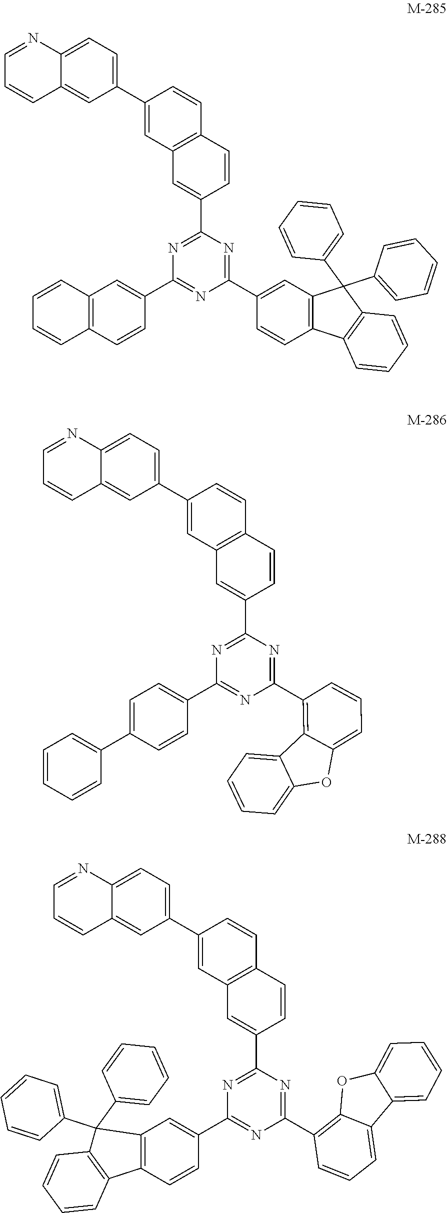 [SMILES: c1ccc(C2(c3ccccc3)c3ccccc3-c3ccc(-c4nc(-c5ccc6ccccc6c5)nc(-c5ccc6ccc(-c7ccc8ncccc8c7)cc6c5)n4)cc32)cc1 ; c1ccc(-c2ccc(-c3nc(-c4ccc5ccc(-c6ccc7ncccc7c6)cc5c4)nc(-c4cccc5oc6ccccc6c45)n3)cc2)cc1 ; c1ccc(C2(c3ccccc3)c3ccccc3-c3ccc(-c4nc(-c5ccc6ccc(-c7ccc8ncccc8c7)cc6c5)nc(-c5cccc6c5oc5ccccc56)n4)cc32)cc1]

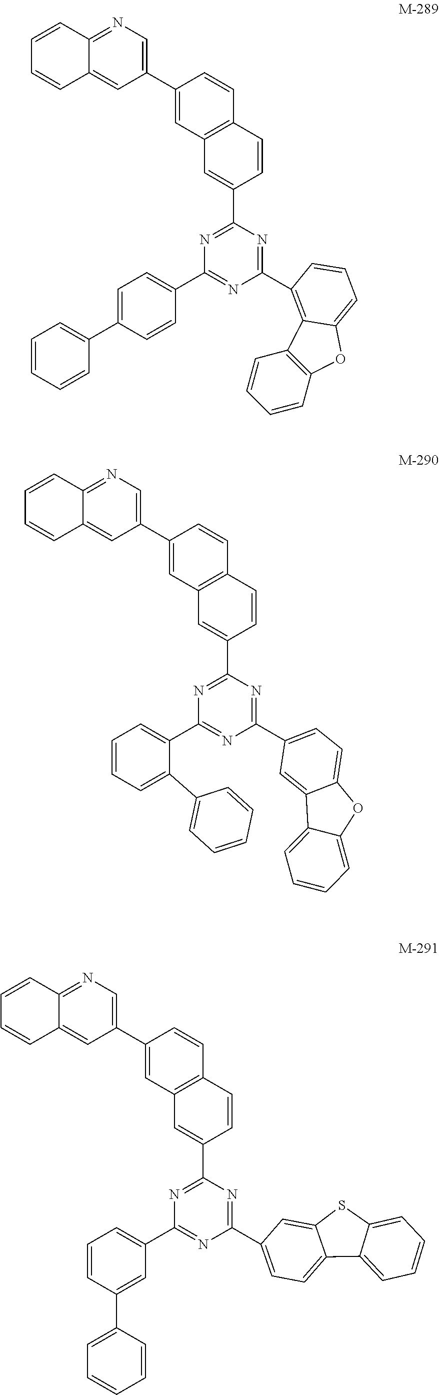 [SMILES: c1ccc(-c2ccc(-c3nc(-c4ccc5ccc(-c6cnc7ccccc7c6)cc5c4)nc(-c4cccc5oc6ccccc6c45)n3)cc2)cc1 ; c1ccc(-c2ccccc2-c2nc(-c3ccc4ccc(-c5cnc6ccccc6c5)cc4c3)nc(-c3ccc4oc5ccccc5c4c3)n2)cc1 ; c1ccc(-c2cccc(-c3nc(-c4ccc5ccc(-c6cnc7ccccc7c6)cc5c4)nc(-c4ccc5c(c4)sc4ccccc45)n3)c2)cc1]

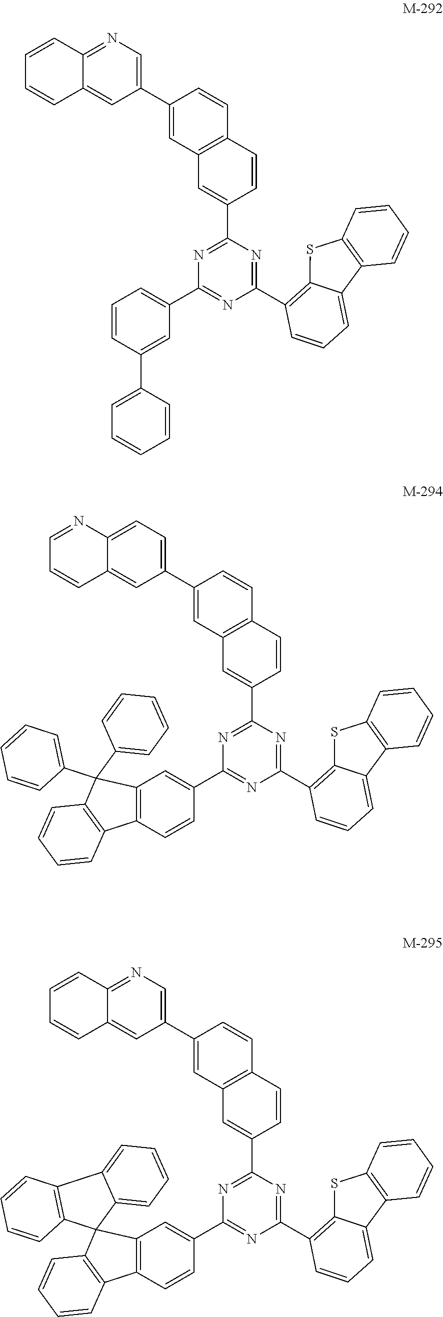 [SMILES: c1ccc(-c2cccc(-c3nc(-c4ccc5ccc(-c6cnc7ccccc7c6)cc5c4)nc(-c4cccc5c4sc4ccccc45)n3)c2)cc1 ; c1ccc(C2(c3ccccc3)c3ccccc3-c3ccc(-c4nc(-c5ccc6ccc(-c7ccc8ncccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6cnc7ccccc7c6)cc5c4)nc(-c4cccc5c4sc4ccccc45)n3)cc21]

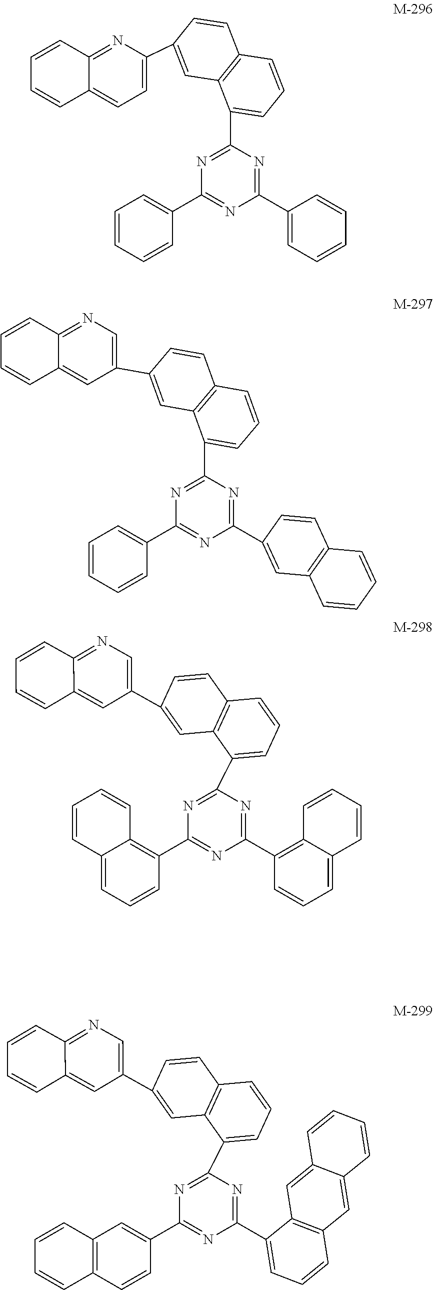 [SMILES: c1ccc(-c2nc(-c3ccccc3)nc(-c3cccc4ccc(-c5ccc6ccccc6n5)cc34)n2)cc1 ; c1ccc(-c2nc(-c3ccc4ccccc4c3)nc(-c3cccc4ccc(-c5cnc6ccccc6c5)cc34)n2)cc1 ; c1ccc2ncc(-c3ccc4cccc(-c5nc(-c6cccc7ccccc67)nc(-c6cccc7ccccc67)n5)c4c3)cc2c1 ; c1ccc2cc(-c3nc(-c4cccc5ccc(-c6cnc7ccccc7c6)cc45)nc(-c4cccc5cc6ccccc6cc45)n3)ccc2c1]

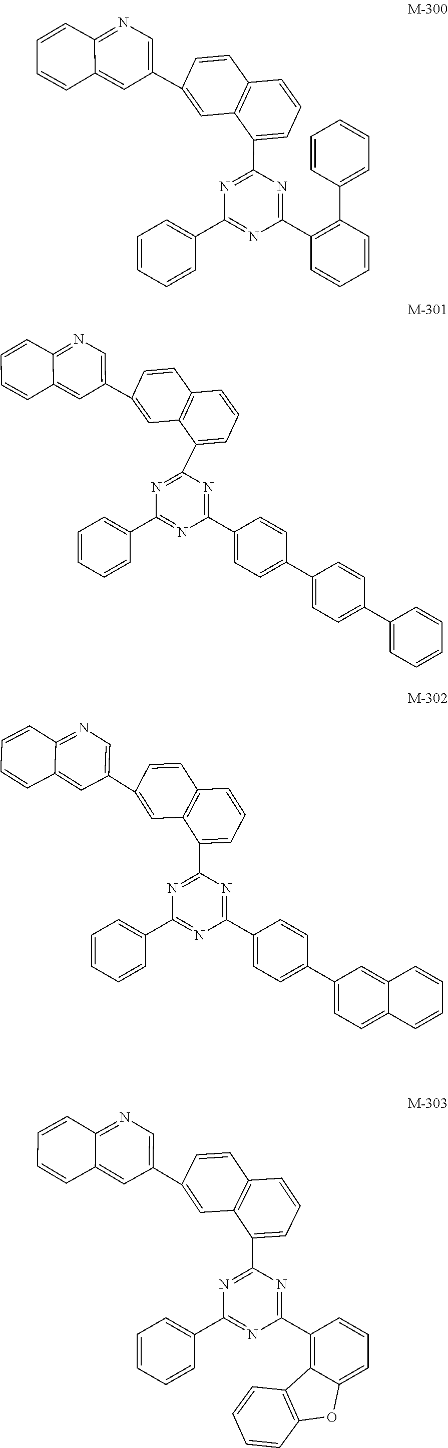 [SMILES: c1ccc(-c2nc(-c3ccccc3-c3ccccc3)nc(-c3cccc4ccc(-c5cnc6ccccc6c5)cc34)n2)cc1 ; c1ccc(-c2ccc(-c3ccc(-c4nc(-c5ccccc5)nc(-c5cccc6ccc(-c7cnc8ccccc8c7)cc56)n4)cc3)cc2)cc1 ; c1ccc(-c2nc(-c3ccc(-c4ccc5ccccc5c4)cc3)nc(-c3cccc4ccc(-c5cnc6ccccc6c5)cc34)n2)cc1 ; c1ccc(-c2nc(-c3cccc4ccc(-c5cnc6ccccc6c5)cc34)nc(-c3cccc4oc5ccccc5c34)n2)cc1]

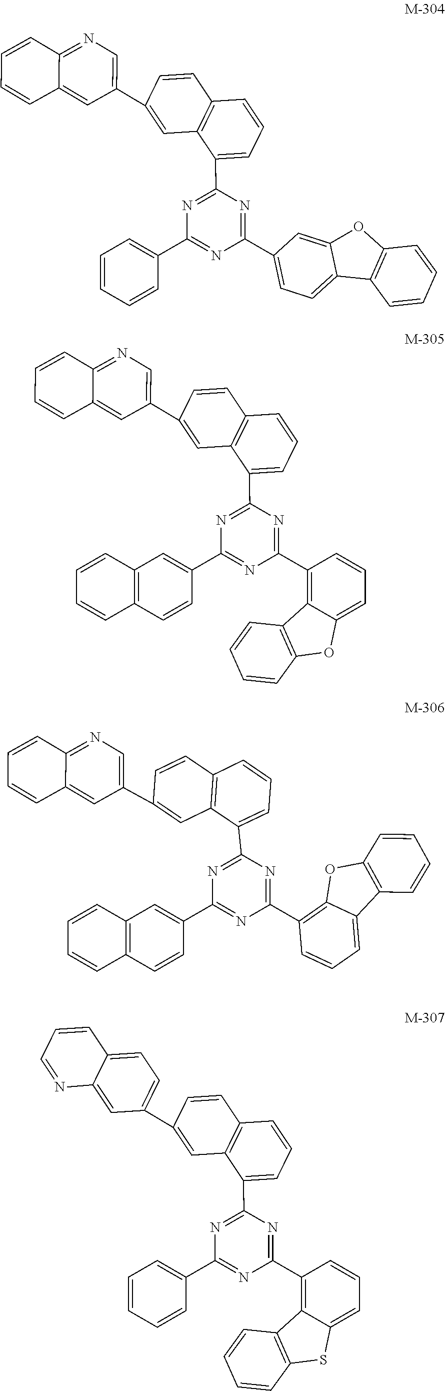 [SMILES: c1ccc(-c2nc(-c3ccc4c(c3)oc3ccccc34)nc(-c3cccc4ccc(-c5cnc6ccccc6c5)cc34)n2)cc1 ; c1ccc2cc(-c3nc(-c4cccc5ccc(-c6cnc7ccccc7c6)cc45)nc(-c4cccc5oc6ccccc6c45)n3)ccc2c1 ; c1ccc2cc(-c3nc(-c4cccc5ccc(-c6cnc7ccccc7c6)cc45)nc(-c4cccc5c4oc4ccccc45)n3)ccc2c1 ; c1ccc(-c2nc(-c3cccc4ccc(-c5ccc6cccnc6c5)cc34)nc(-c3cccc4sc5ccccc5c34)n2)cc1]

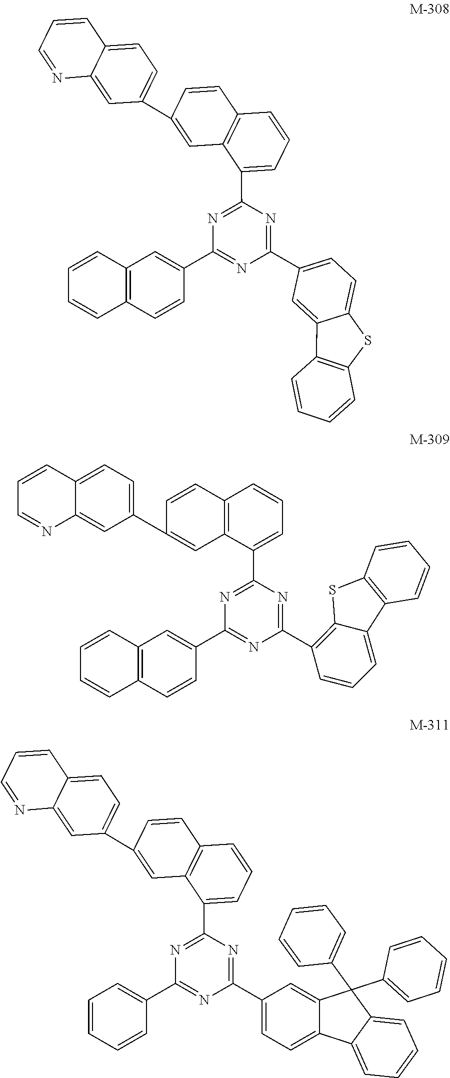 [SMILES: c1ccc2cc(-c3nc(-c4ccc5sc6ccccc6c5c4)nc(-c4cccc5ccc(-c6ccc7cccnc7c6)cc45)n3)ccc2c1 ; c1ccc2cc(-c3nc(-c4cccc5ccc(-c6ccc7cccnc7c6)cc45)nc(-c4cccc5c4sc4ccccc45)n3)ccc2c1 ; c1ccc(-c2nc(-c3ccc4c(c3)C(c3ccccc3)(c3ccccc3)c3ccccc3-4)nc(-c3cccc4ccc(-c5ccc6cccnc6c5)cc34)n2)cc1]

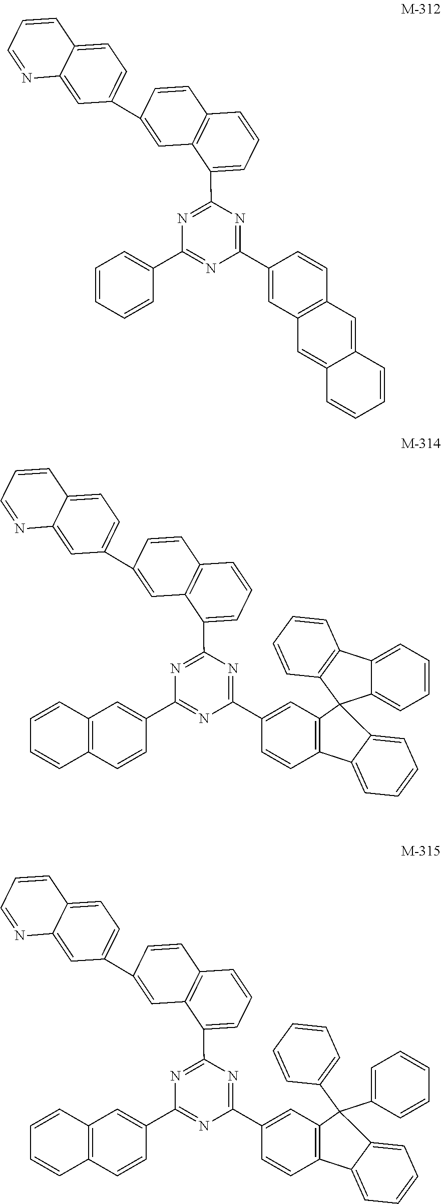 [SMILES: c1ccc(-c2nc(-c3ccc4cc5ccccc5cc4c3)nc(-c3cccc4ccc(-c5ccc6cccnc6c5)cc34)n2)cc1 ; c1ccc2c(c1)-c1ccccc1C21c2ccccc2-c2ccc(-c3nc(-c4ccc5ccccc5c4)nc(-c4cccc5ccc(-c6ccc7cccnc7c6)cc45)n3)cc21 ; c1ccc(C2(c3ccccc3)c3ccccc3-c3ccc(-c4nc(-c5ccc6ccccc6c5)nc(-c5cccc6ccc(-c7ccc8cccnc8c7)cc56)n4)cc32)cc1]

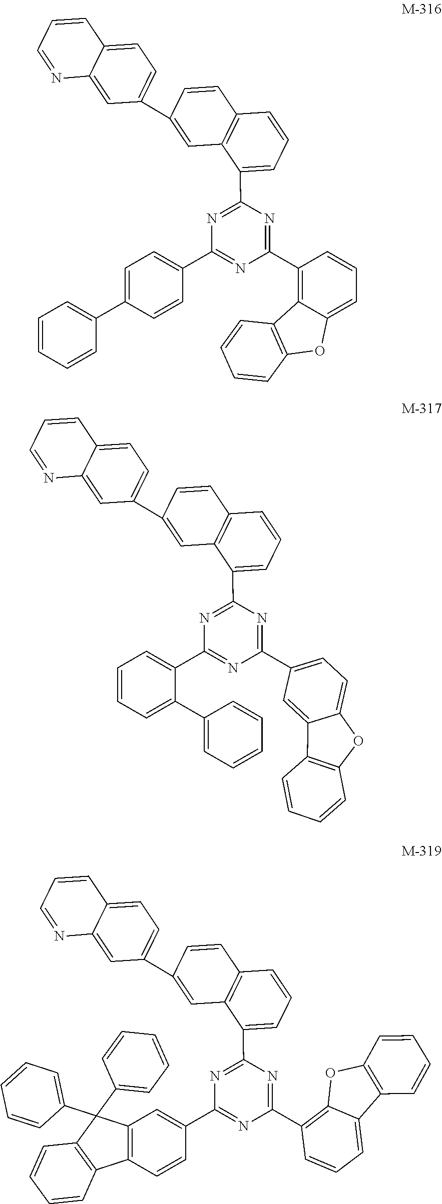 [SMILES: c1ccc(-c2ccc(-c3nc(-c4cccc5ccc(-c6ccc7cccnc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6cccnc6c5)cc34)n2)cc1 ; c1ccc(C2(c3ccccc3)c3ccccc3-c3ccc(-c4nc(-c5cccc6ccc(-c7ccc8cccnc8c7)cc56)nc(-c5cccc6c5oc5ccccc56)n4)cc32)cc1]

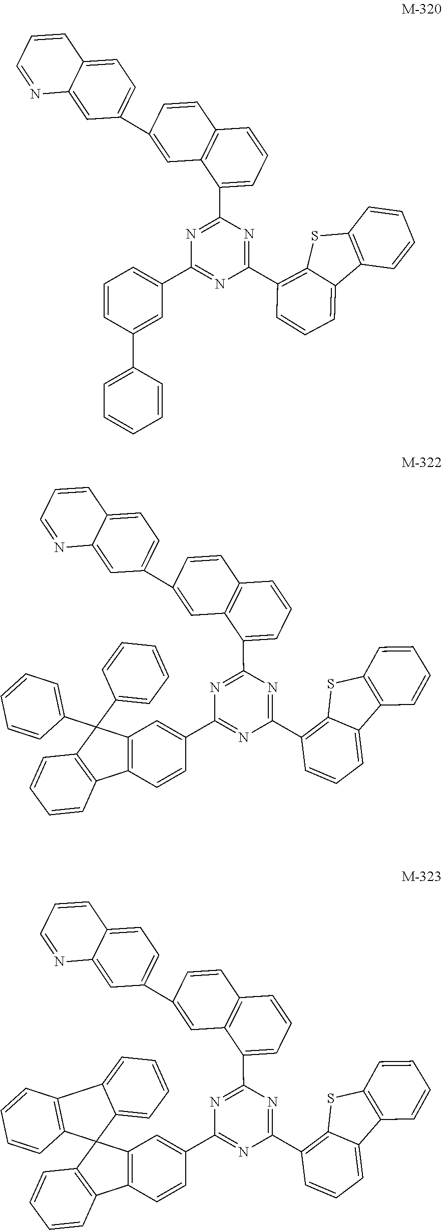 [SMILES: c1ccc(-c2cccc(-c3nc(-c4cccc5ccc(-c6ccc7cccnc7c6)cc45)nc(-c4cccc5c4sc4ccccc45)n3)c2)cc1 ; c1ccc(C2(c3ccccc3)c3ccccc3-c3ccc(-c4nc(-c5cccc6ccc(-c7ccc8cccnc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7cccnc7c6)cc45)nc(-c4cccc5c4sc4ccccc45)n3)cc21]

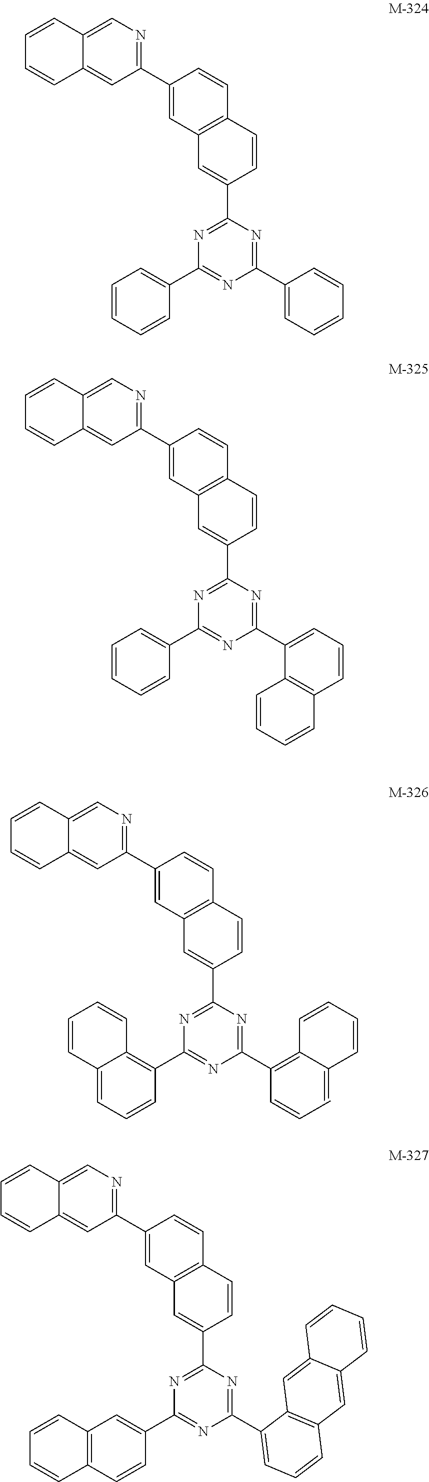 [SMILES: c1ccc(-c2nc(-c3ccccc3)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3cccc4ccccc34)n2)cc1 ; C=c1cccc/c1=C(/C=C\C)c1nc(-c2ccc3ccc(-c4cc5ccccc5cn4)cc3c2)nc(-c2cccc3ccccc23)n1 ; c1ccc2cc(-c3nc(-c4ccc5ccc(-c6cc7ccccc7cn6)cc5c4)nc(-c4cccc5cc6ccccc6cc45)n3)ccc2c1]

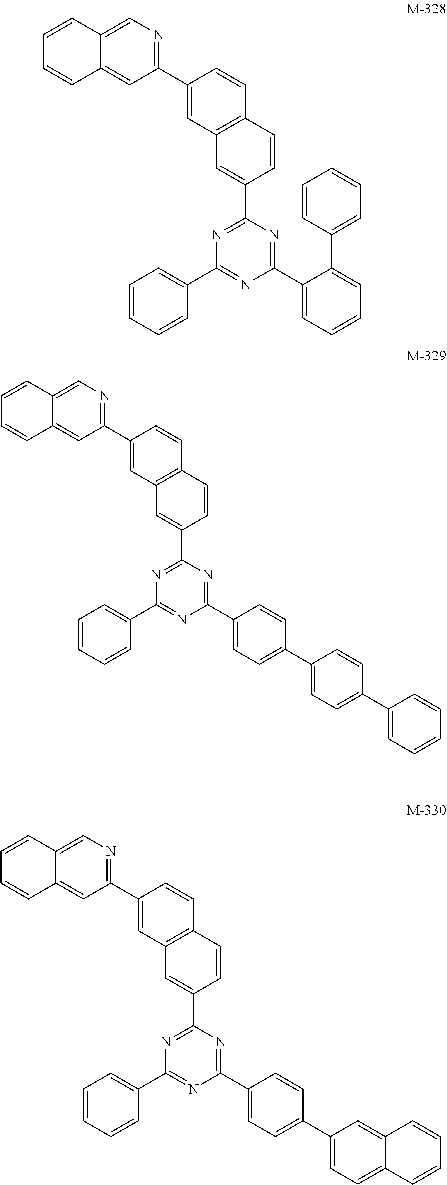 [SMILES: c1ccc(-c2nc(-c3ccc4ccc(-c5cc6ccccc6cn5)cc4c3)nc(-c3ccccc3-c3ccccc3)n2)cc1 ; c1ccc(-c2ccc(-c3ccc(-c4nc(-c5ccccc5)nc(-c5ccc6ccc(-c7cc8ccccc8cn7)cc6c5)n4)cc3)cc2)cc1 ; c1ccc(-c2nc(-c3ccc(-c4ccc5ccccc5c4)cc3)nc(-c3ccc4ccc(-c5cc6ccccc6cn5)cc4c3)n2)cc1]

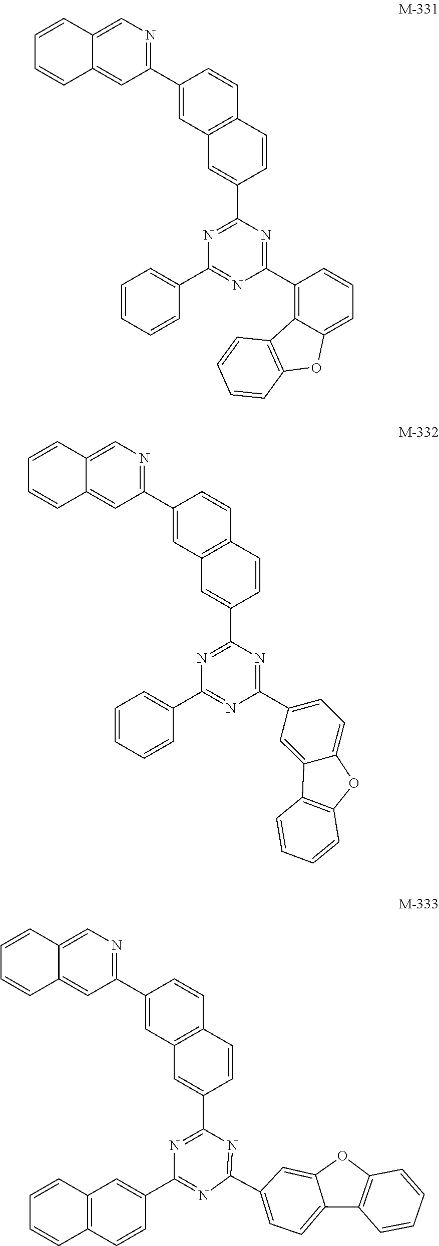 [SMILES: c1ccc(-c2nc(-c3ccc4ccc(-c5cc6ccccc6cn5)cc4c3)nc(-c3cccc4oc5ccccc5c34)n2)cc1 ; c1ccc(-c2nc(-c3ccc4ccc(-c5cc6ccccc6cn5)cc4c3)nc(-c3ccc4oc5ccccc5c4c3)n2)cc1 ; c1ccc2cc(-c3nc(-c4ccc5ccc(-c6cc7ccccc7cn6)cc5c4)nc(-c4ccc5c(c4)oc4ccccc45)n3)ccc2c1]

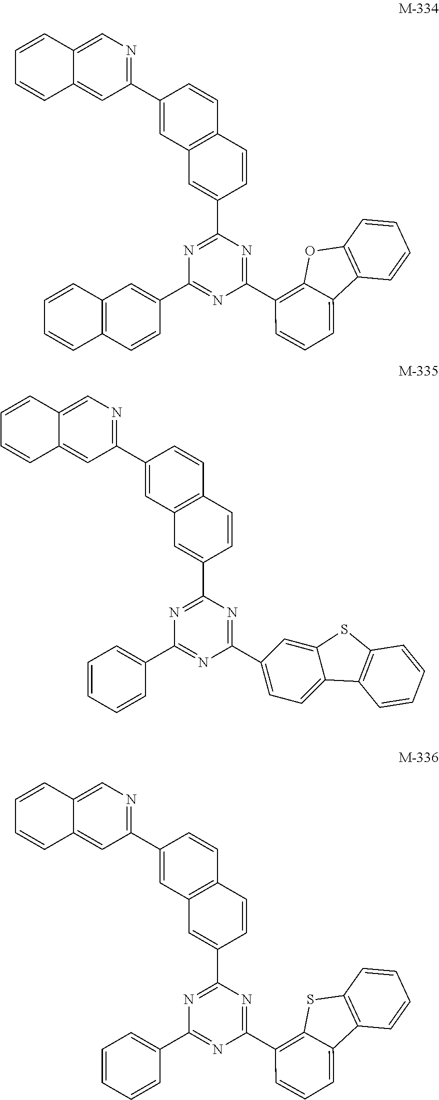 [SMILES: c1ccc2cc(-c3nc(-c4ccc5ccc(-c6cc7ccccc7cn6)cc5c4)nc(-c4cccc5c4oc4ccccc45)n3)ccc2c1 ; c1ccc(-c2nc(-c3ccc4ccc(-c5cc6ccccc6cn5)cc4c3)nc(-c3ccc4c(c3)sc3ccccc34)n2)cc1 ; c1ccc(-c2nc(-c3ccc4ccc(-c5cc6ccccc6cn5)cc4c3)nc(-c3cccc4c3sc3ccccc34)n2)cc1]

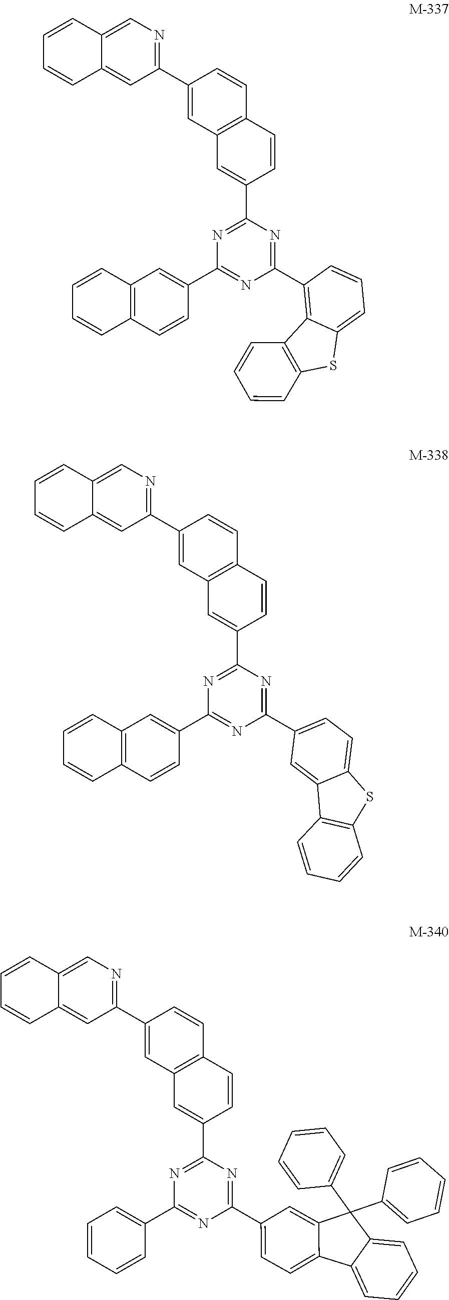 [SMILES: c1ccc2cc(-c3nc(-c4ccc5ccc(-c6cc7ccccc7cn6)cc5c4)nc(-c4cccc5sc6ccccc6c45)n3)ccc2c1 ; c1ccc2cc(-c3nc(-c4ccc5ccc(-c6cc7ccccc7cn6)cc5c4)nc(-c4ccc5sc6ccccc6c5c4)n3)ccc2c1 ; c1ccc(-c2nc(-c3ccc4c(c3)C(c3ccccc3)(c3ccccc3)c3ccccc3-4)nc(-c3ccc4ccc(-c5cc6ccccc6cn5)cc4c3)n2)cc1]

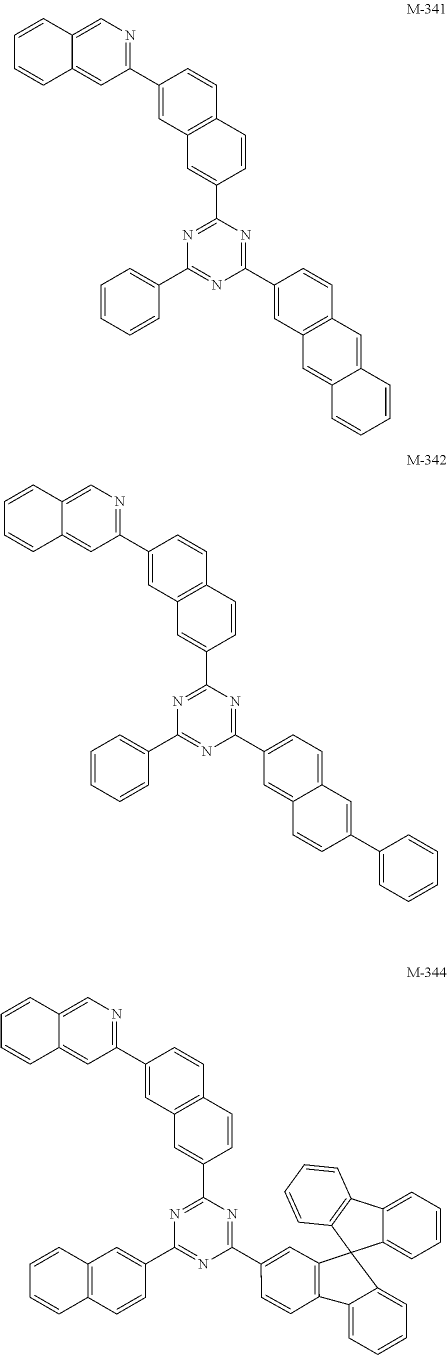 [SMILES: c1ccc(-c2nc(-c3ccc4ccc(-c5cc6ccccc6cn5)cc4c3)nc(-c3ccc4cc5ccccc5cc4c3)n2)cc1 ; c1ccc(-c2ccc3cc(-c4nc(-c5ccccc5)nc(-c5ccc6ccc(-c7cc8ccccc8cn7)cc6c5)n4)ccc3c2)cc1 ; c1ccc2c(c1)-c1ccccc1C21c2ccccc2-c2ccc(-c3nc(-c4ccc5ccccc5c4)nc(-c4ccc5ccc(-c6cc7ccccc7cn6)cc5c4)n3)cc21]

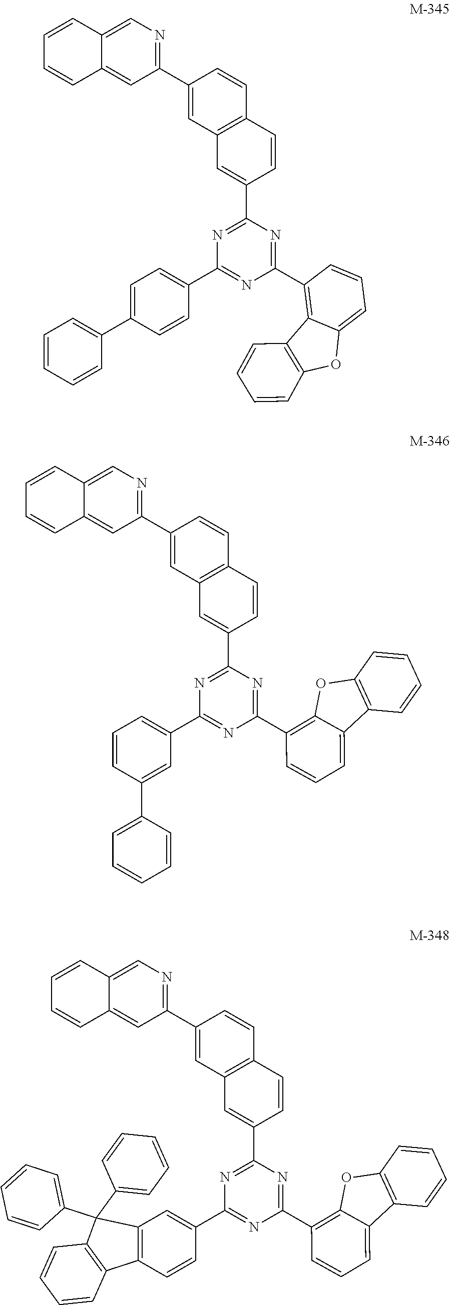 [SMILES: c1ccc(-c2ccc(-c3nc(-c4ccc5ccc(-c6cc7ccccc7cn6)cc5c4)nc(-c4cccc5oc6ccccc6c45)n3)cc2)cc1 ; c1ccc(-c2cccc(-c3nc(-c4ccc5ccc(-c6cc7ccccc7cn6)cc5c4)nc(-c4cccc5c4oc4ccccc45)n3)c2)cc1 ; c1ccc(C2(c3ccccc3)c3ccccc3-c3ccc(-c4nc(-c5ccc6ccc(-c7cc8ccccc8cn7)cc6c5)nc(-c5cccc6c5oc5ccccc56)n4)cc32)cc1]

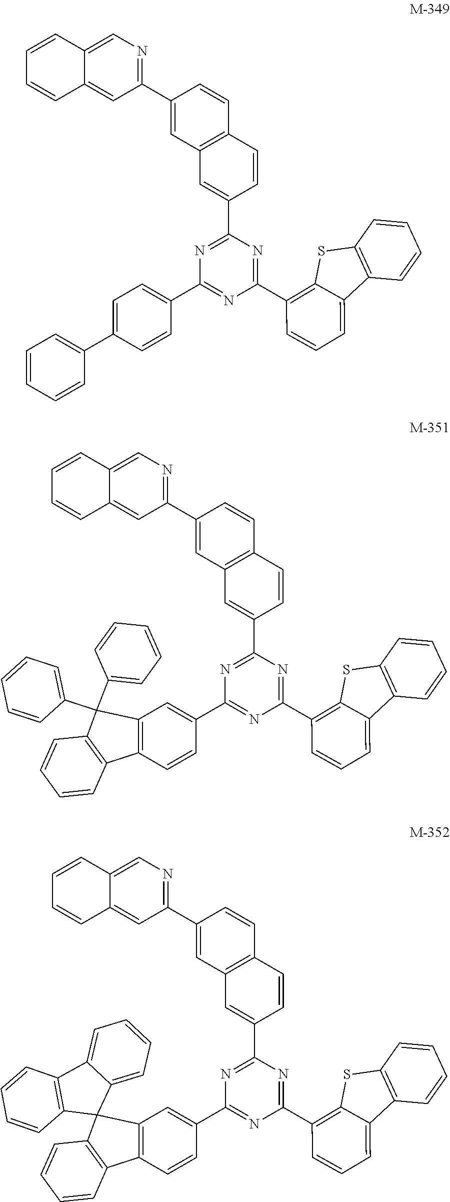 [SMILES: c1ccc(-c2ccc(-c3nc(-c4ccc5ccc(-c6cc7ccccc7cn6)cc5c4)nc(-c4cccc5c4sc4ccccc45)n3)cc2)cc1 ; c1ccc(C2(c3ccccc3)c3ccccc3-c3ccc(-c4nc(-c5ccc6ccc(-c7cc8ccccc8cn7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6cc7ccccc7cn6)cc5c4)nc(-c4cccc5c4sc4ccccc45)n3)cc21]

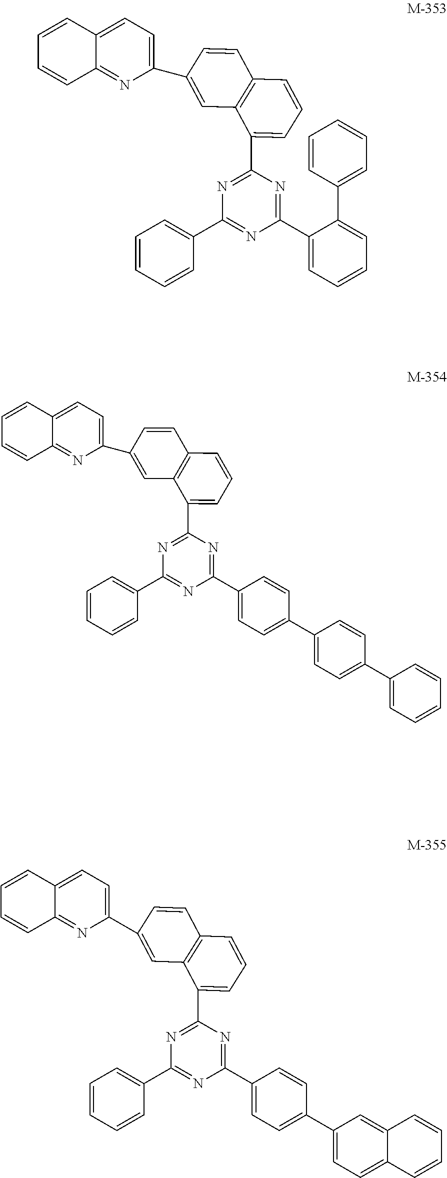 [SMILES: c1ccc(-c2nc(-c3ccccc3-c3ccccc3)nc(-c3cccc4ccc(-c5ccc6ccccc6n5)cc34)n2)cc1 ; c1ccc(-c2ccc(-c3ccc(-c4nc(-c5ccccc5)nc(-c5cccc6ccc(-c7ccc8ccccc8n7)cc56)n4)cc3)cc2)cc1 ; c1ccc(-c2nc(-c3ccc(-c4ccc5ccccc5c4)cc3)nc(-c3cccc4ccc(-c5ccc6ccccc6n5)cc34)n2)cc1]

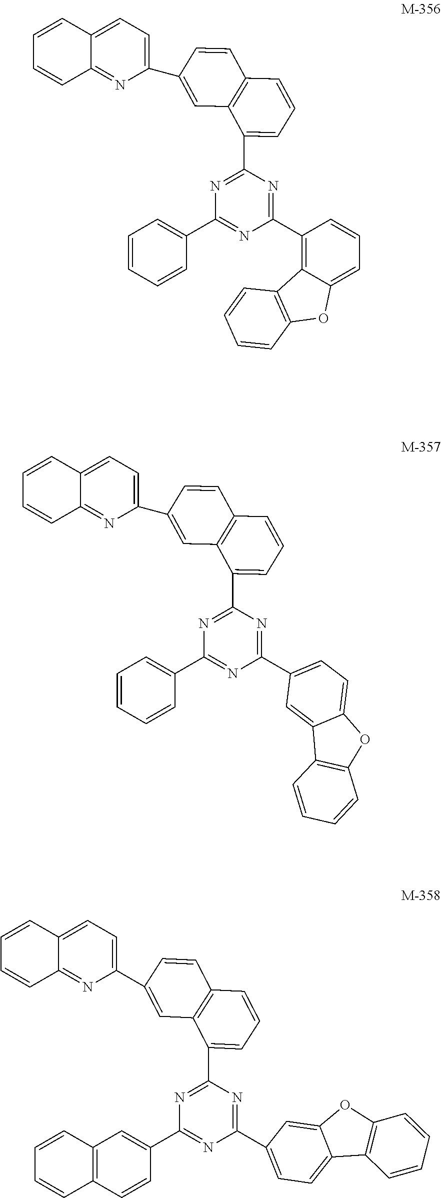 [SMILES: c1ccc(-c2nc(-c3cccc4ccc(-c5ccc6ccccc6n5)cc34)nc(-c3cccc4oc5ccccc5c34)n2)cc1 ; c1ccc(-c2nc(-c3ccc4oc5ccccc5c4c3)nc(-c3cccc4ccc(-c5ccc6ccccc6n5)cc34)n2)cc1 ; c1ccc2cc(-c3nc(-c4ccc5c(c4)oc4ccccc45)nc(-c4cccc5ccc(-c6ccc7ccccc7n6)cc45)n3)ccc2c1]

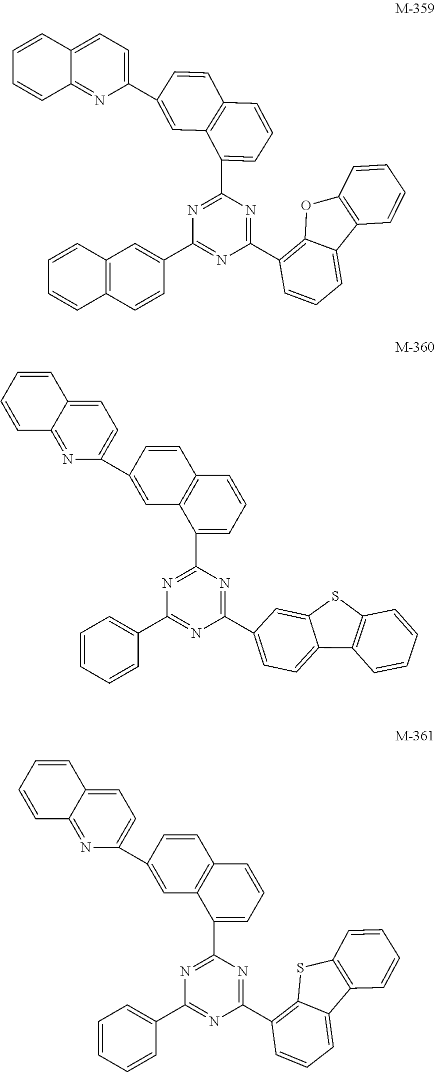 [SMILES: c1ccc2cc(-c3nc(-c4cccc5ccc(-c6ccc7ccccc7n6)cc45)nc(-c4cccc5c4oc4ccccc45)n3)ccc2c1 ; c1ccc(-c2nc(-c3ccc4c(c3)sc3ccccc34)nc(-c3cccc4ccc(-c5ccc6ccccc6n5)cc34)n2)cc1 ; c1ccc(-c2nc(-c3cccc4ccc(-c5ccc6ccccc6n5)cc34)nc(-c3cccc4c3sc3ccccc34)n2)cc1]

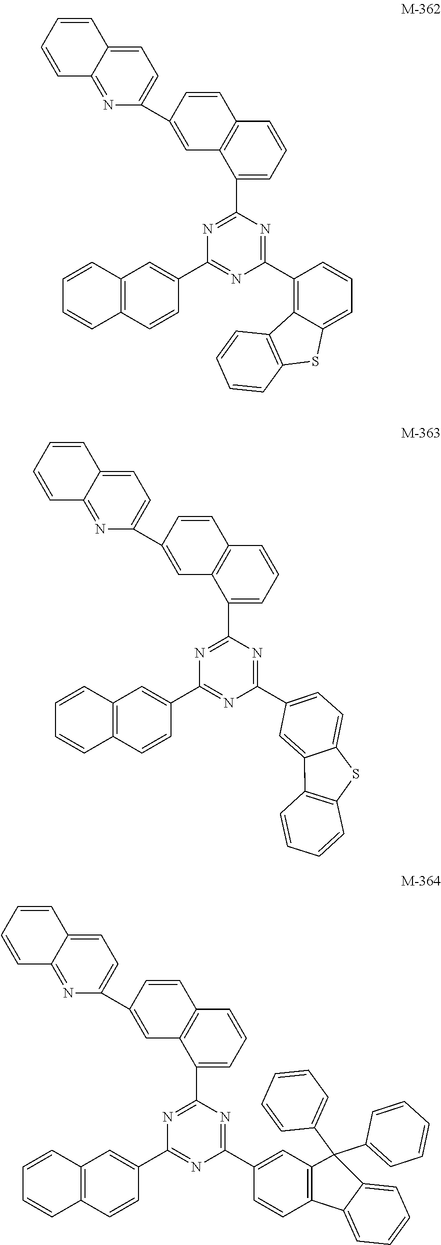 [SMILES: c1ccc2cc(-c3nc(-c4cccc5ccc(-c6ccc7ccccc7n6)cc45)nc(-c4cccc5sc6ccccc6c45)n3)ccc2c1 ; c1ccc2cc(-c3nc(-c4ccc5sc6ccccc6c5c4)nc(-c4cccc5ccc(-c6ccc7ccccc7n6)cc45)n3)ccc2c1 ; c1ccc(C2(c3ccccc3)c3ccccc3-c3ccc(-c4nc(-c5ccc6ccccc6c5)nc(-c5cccc6ccc(-c7ccc8ccccc8n7)cc56)n4)cc32)cc1]

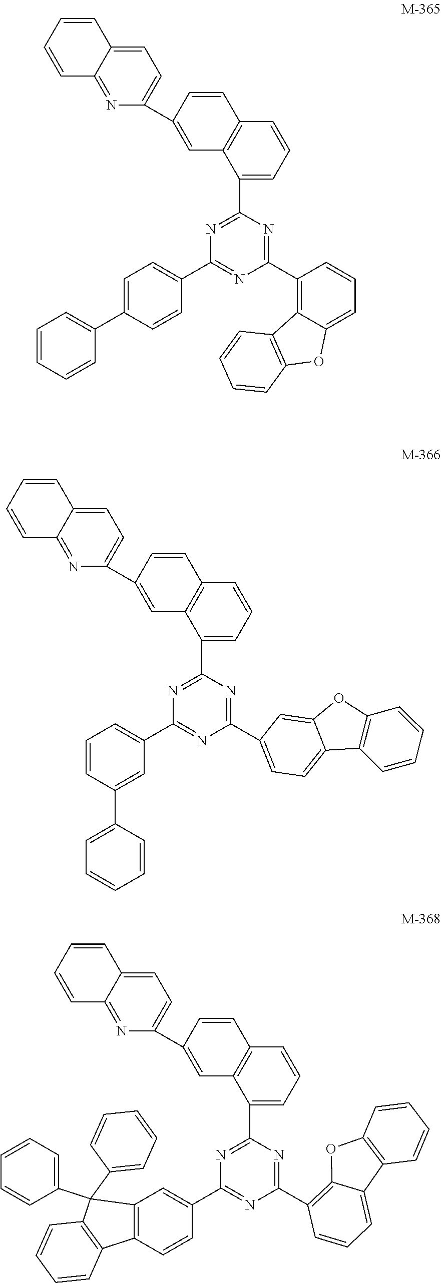 [SMILES: c1ccc(-c2ccc(-c3nc(-c4cccc5ccc(-c6ccc7ccccc7n6)cc45)nc(-c4cccc5oc6ccccc6c45)n3)cc2)cc1 ; c1ccc(-c2cccc(-c3nc(-c4ccc5c(c4)oc4ccccc45)nc(-c4cccc5ccc(-c6ccc7ccccc7n6)cc45)n3)c2)cc1 ; c1ccc(C2(c3ccccc3)c3ccccc3-c3ccc(-c4nc(-c5cccc6ccc(-c7ccc8ccccc8n7)cc56)nc(-c5cccc6c5oc5ccccc56)n4)cc32)cc1]

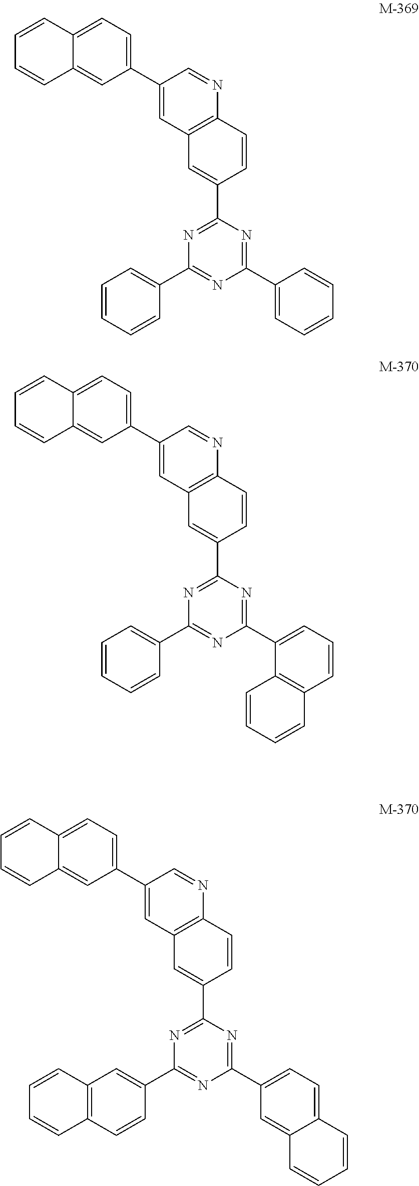 [SMILES: c1ccc(-c2nc(-c3ccccc3)nc(-c3ccc4ncc(-c5ccc6ccccc6c5)cc4c3)n2)cc1 ; c1ccc(-c2nc(-c3ccc4ncc(-c5ccc6ccccc6c5)cc4c3)nc(-c3cccc4ccccc34)n2)cc1 ; c1ccc2cc(-c3cnc4ccc(-c5nc(-c6ccc7ccccc7c6)nc(-c6ccc7ccccc7c6)n5)cc4c3)ccc2c1]

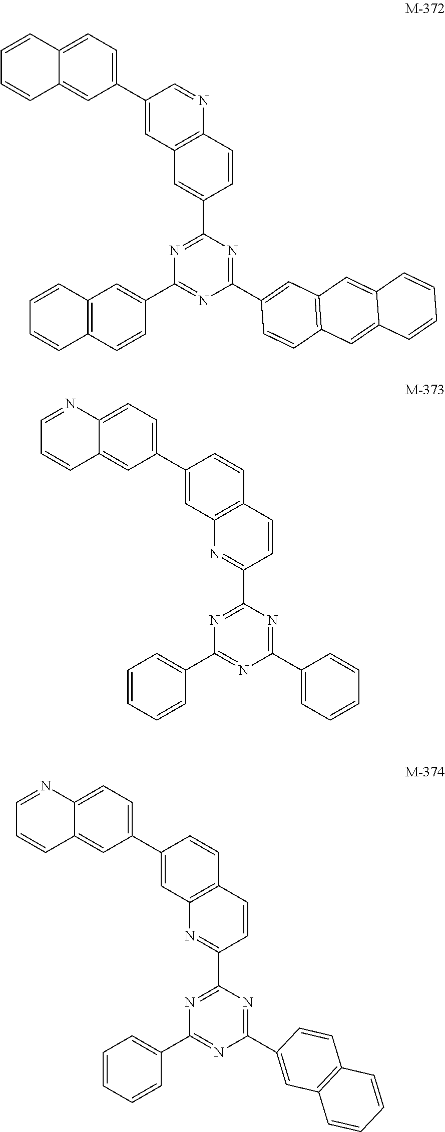 [SMILES: c1ccc2cc(-c3cnc4ccc(-c5nc(-c6ccc7ccccc7c6)nc(-c6ccc7cc8ccccc8cc7c6)n5)cc4c3)ccc2c1 ; c1ccc(-c2nc(-c3ccccc3)nc(-c3ccc4ccc(-c5ccc6ncccc6c5)cc4n3)n2)cc1 ; c1ccc(-c2nc(-c3ccc4ccccc4c3)nc(-c3ccc4ccc(-c5ccc6ncccc6c5)cc4n3)n2)cc1]

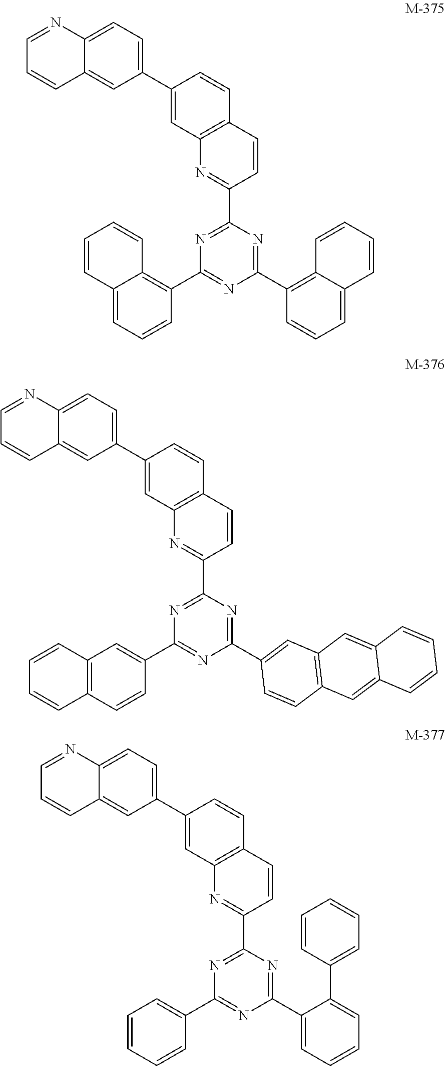

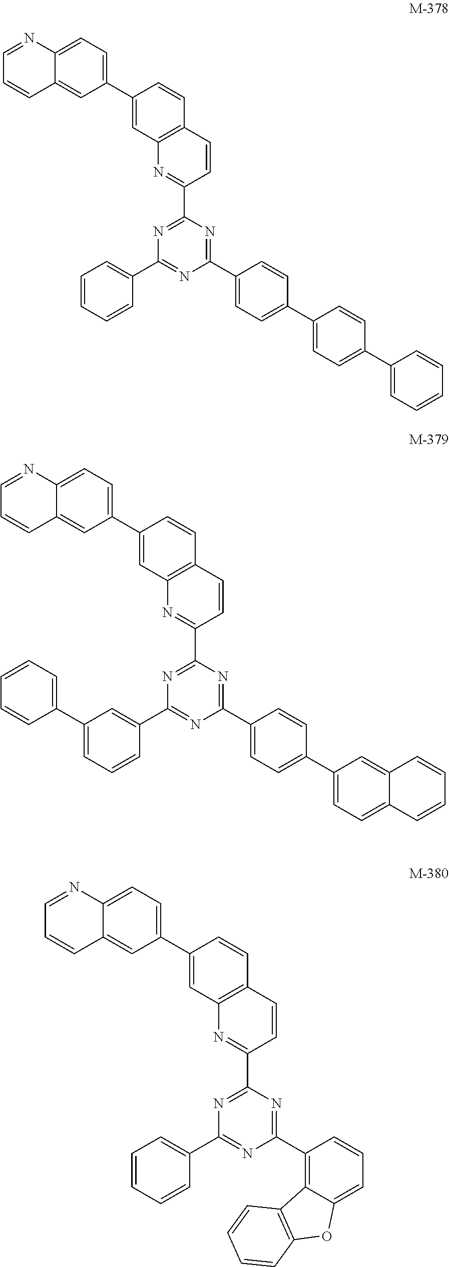 [SMILES: c1ccc(-c2ccc(-c3ccc(-c4nc(-c5ccccc5)nc(-c5ccc6ccc(-c7ccc8ncccc8c7)cc6n5)n4)cc3)cc2)cc1 ; c1ccc(-c2cccc(-c3nc(-c4ccc(-c5ccc6ccccc6c5)cc4)nc(-c4ccc5ccc(-c6ccc7ncccc7c6)cc5n4)n3)c2)cc1 ; c1ccc(-c2nc(-c3ccc4ccc(-c5ccc6ncccc6c5)cc4n3)nc(-c3cccc4oc5ccccc5c34)n2)cc1]

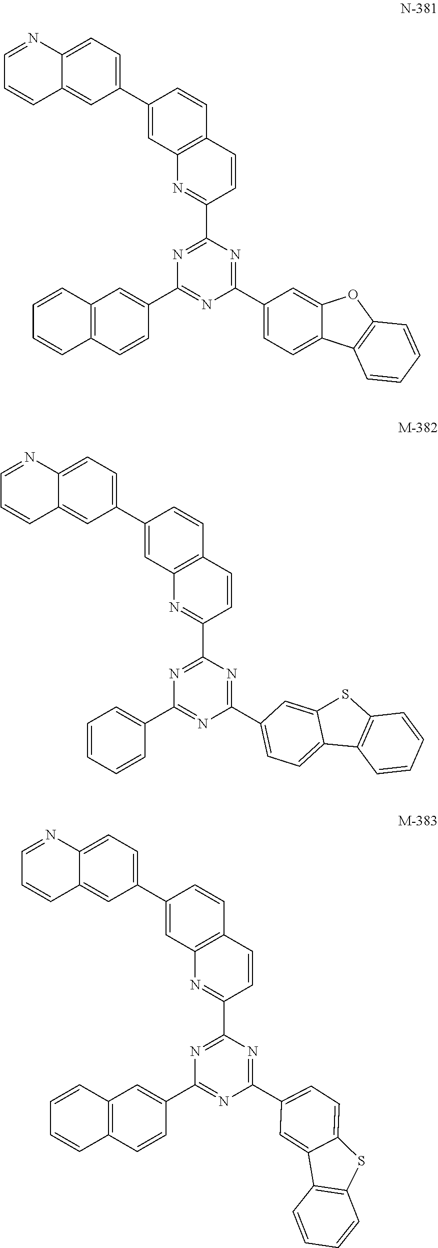 [SMILES: c1ccc2cc(-c3nc(-c4ccc5c(c4)oc4ccccc45)nc(-c4ccc5ccc(-c6ccc7ncccc7c6)cc5n4)n3)ccc2c1 ; c1ccc(-c2nc(-c3ccc4c(c3)sc3ccccc34)nc(-c3ccc4ccc(-c5ccc6ncccc6c5)cc4n3)n2)cc1 ; c1ccc2cc(-c3nc(-c4ccc5sc6ccccc6c5c4)nc(-c4ccc5ccc(-c6ccc7ncccc7c6)cc5n4)n3)ccc2c1]

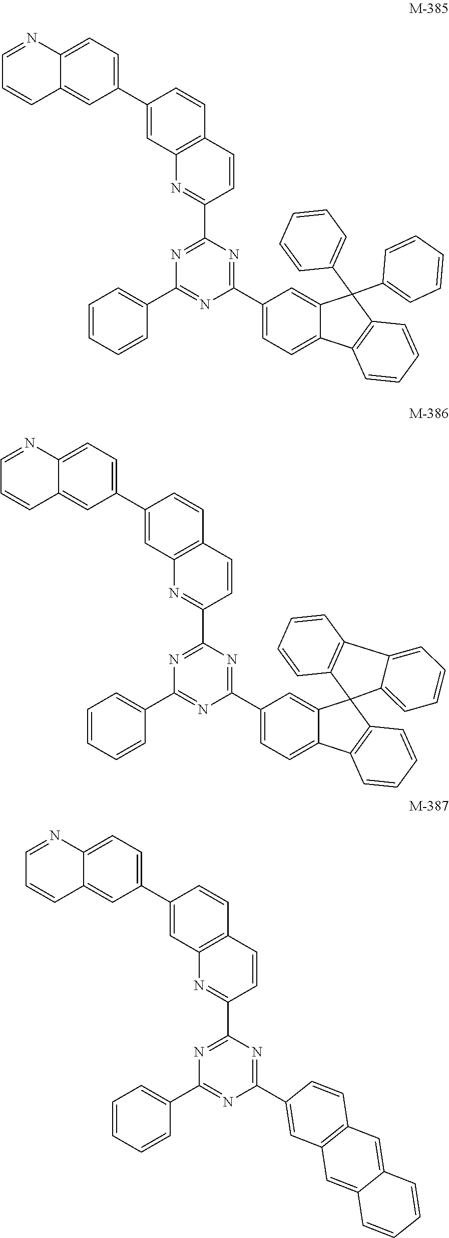 [SMILES: c1ccc(-c2nc(-c3ccc4c(c3)C(c3ccccc3)(c3ccccc3)c3ccccc3-4)nc(-c3ccc4ccc(-c5ccc6ncccc6c5)cc4n3)n2)cc1 ; c1ccc(-c2nc(-c3ccc4c(c3)C3(c5ccccc5-c5ccccc53)c3ccccc3-4)nc(-c3ccc4ccc(-c5ccc6ncccc6c5)cc4n3)n2)cc1 ; c1ccc(-c2nc(-c3ccc4cc5ccccc5cc4c3)nc(-c3ccc4ccc(-c5ccc6ncccc6c5)cc4n3)n2)cc1]

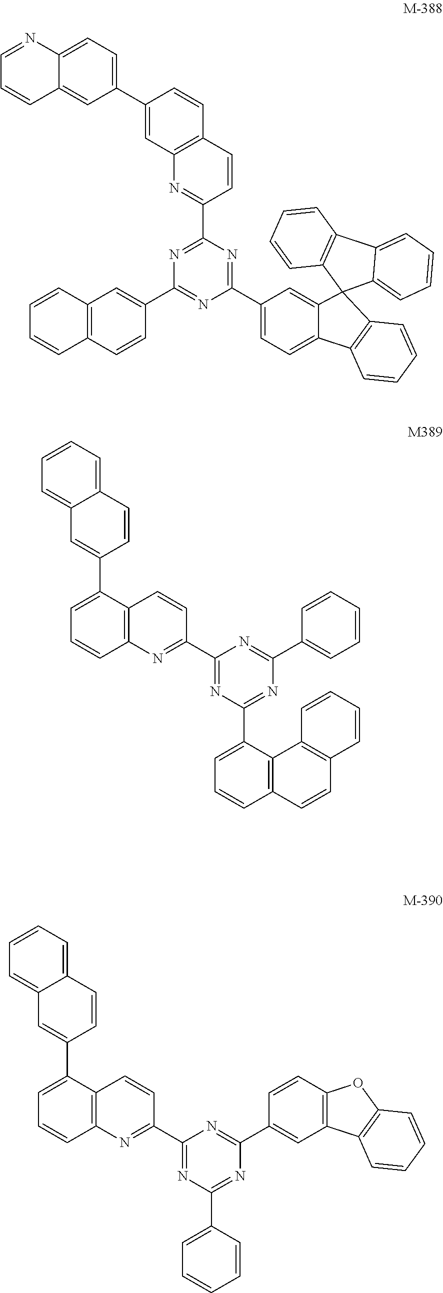 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5n4)n3)cc21 ; c1ccc(-c2nc(-c3ccc4c(-c5ccc6ccccc6c5)cccc4n3)nc(-c3cccc4ccc5ccccc5c34)n2)cc1 ; c1ccc(-c2nc(-c3ccc4oc5ccccc5c4c3)nc(-c3ccc4c(-c5ccc6ccccc6c5)cccc4n3)n2)cc1]

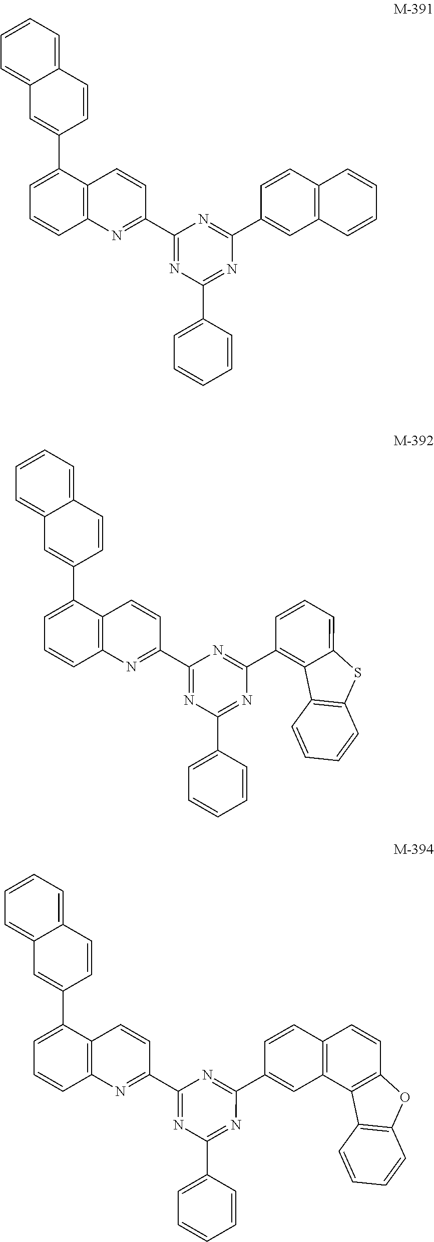 [SMILES: c1ccc(-c2nc(-c3ccc4ccccc4c3)nc(-c3ccc4c(-c5ccc6ccccc6c5)cccc4n3)n2)cc1 ; c1ccc(-c2nc(-c3ccc4c(-c5ccc6ccccc6c5)cccc4n3)nc(-c3cccc4sc5ccccc5c34)n2)cc1 ; c1ccc(-c2nc(-c3ccc4ccc5oc6ccccc6c5c4c3)nc(-c3ccc4c(-c5ccc6ccccc6c5)cccc4n3)n2)cc1]

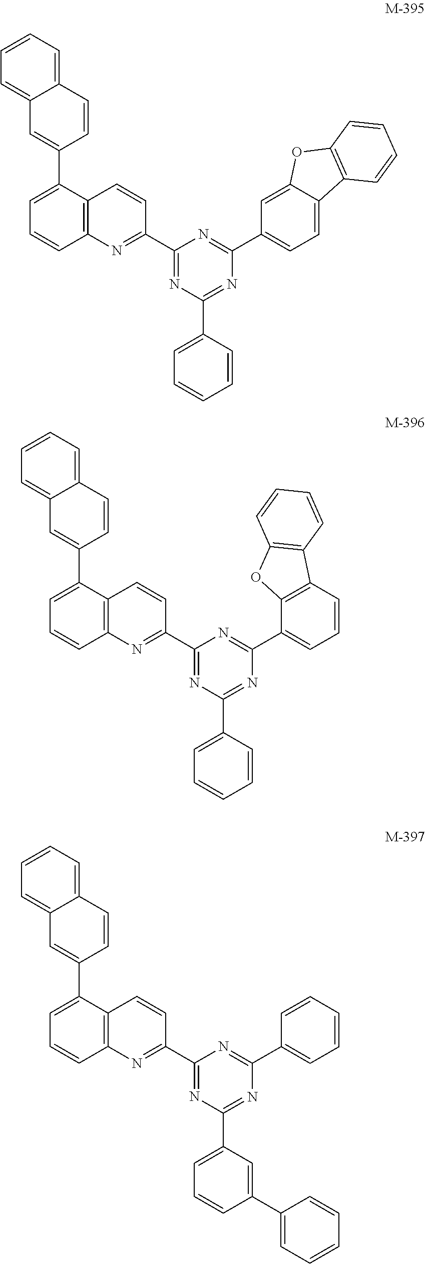 [SMILES: c1ccc(-c2nc(-c3ccc4c(c3)oc3ccccc34)nc(-c3ccc4c(-c5ccc6ccccc6c5)cccc4n3)n2)cc1 ; c1ccc(-c2nc(-c3ccc4c(-c5ccc6ccccc6c5)cccc4n3)nc(-c3cccc4c3oc3ccccc34)n2)cc1 ; c1ccc(-c2cccc(-c3nc(-c4ccccc4)nc(-c4ccc5c(-c6ccc7ccccc7c6)cccc5n4)n3)c2)cc1]

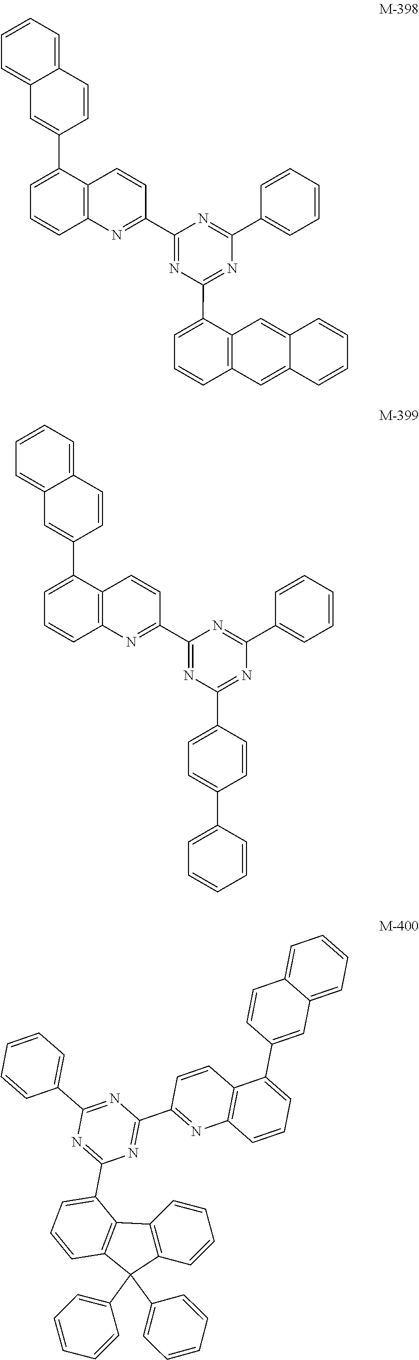 [SMILES: c1ccc(-c2nc(-c3ccc4c(-c5ccc6ccccc6c5)cccc4n3)nc(-c3cccc4cc5ccccc5cc34)n2)cc1 ; c1ccc(-c2ccc(-c3nc(-c4ccccc4)nc(-c4ccc5c(-c6ccc7ccccc7c6)cccc5n4)n3)cc2)cc1 ; c1ccc(-c2nc(-c3ccc4c(-c5ccc6ccccc6c5)cccc4n3)nc(-c3cccc4c3-c3ccccc3C4(c3ccccc3)c3ccccc3)n2)cc1]

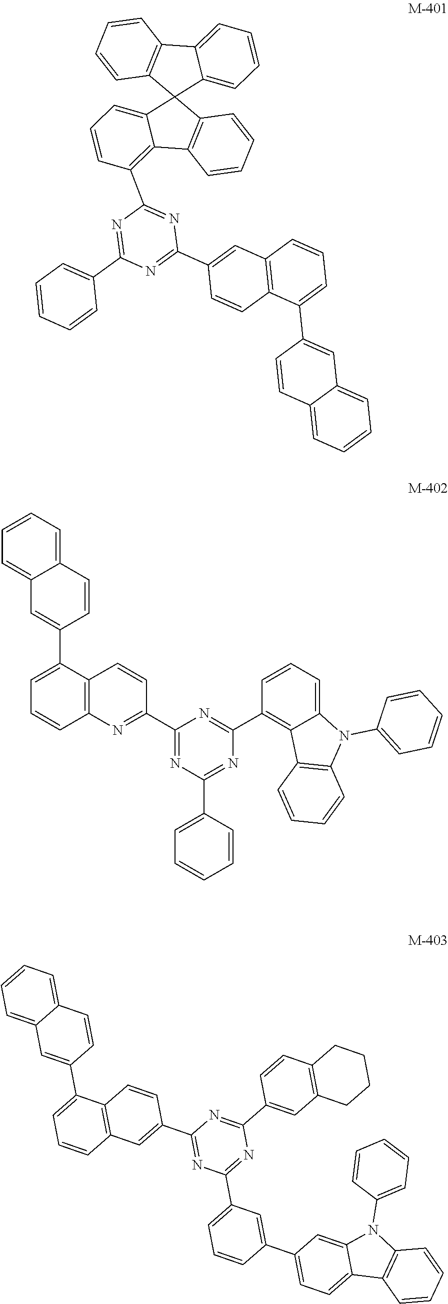 [SMILES: c1ccc(-c2nc(-c3ccc4c(-c5ccc6ccccc6c5)cccc4c3)nc(-c3cccc4c3-c3ccccc3C43c4ccccc4-c4ccccc43)n2)cc1 ; c1ccc(-c2nc(-c3ccc4c(-c5ccc6ccccc6c5)cccc4n3)nc(-c3cccc4c3c3ccccc3n4-c3ccccc3)n2)cc1 ; c1ccc(-n2c3ccccc3c3ccc(-c4cccc(-c5nc(-c6ccc7c(c6)CCCC7)nc(-c6ccc7c(-c8ccc9ccccc9c8)cccc7c6)n5)c4)cc32)cc1]

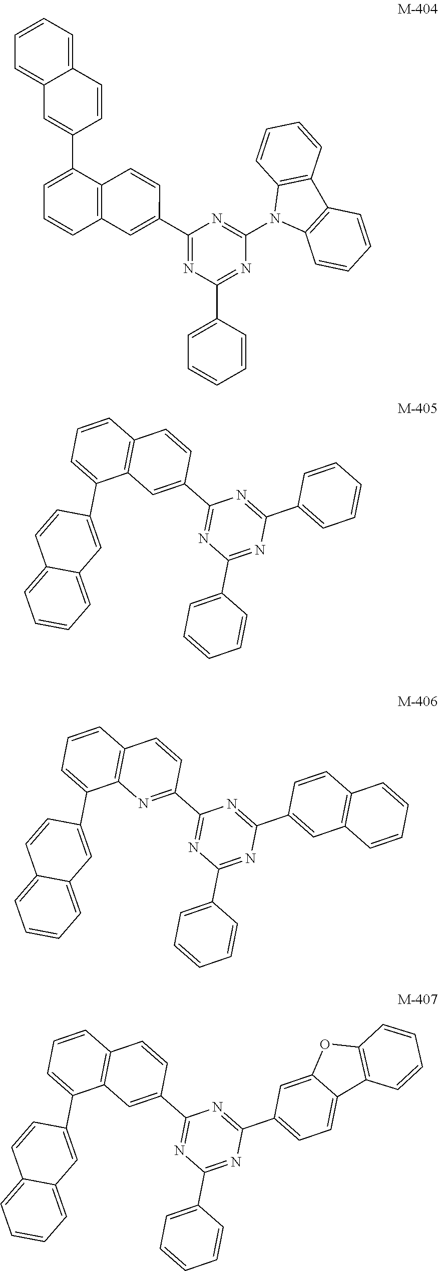 [SMILES: c1ccc(-c2nc(-c3ccc4c(-c5ccc6ccccc6c5)cccc4c3)nc(-n3c4ccccc4c4ccccc43)n2)cc1 ; c1ccc(-c2nc(-c3ccccc3)nc(-c3ccc4cccc(-c5ccc6ccccc6c5)c4c3)n2)cc1 ; c1ccc(-c2nc(-c3ccc4ccccc4c3)nc(-c3ccc4cccc(-c5ccc6ccccc6c5)c4n3)n2)cc1 ; c1ccc(-c2nc(-c3ccc4c(c3)oc3ccccc34)nc(-c3ccc4cccc(-c5ccc6ccccc6c5)c4c3)n2)cc1]

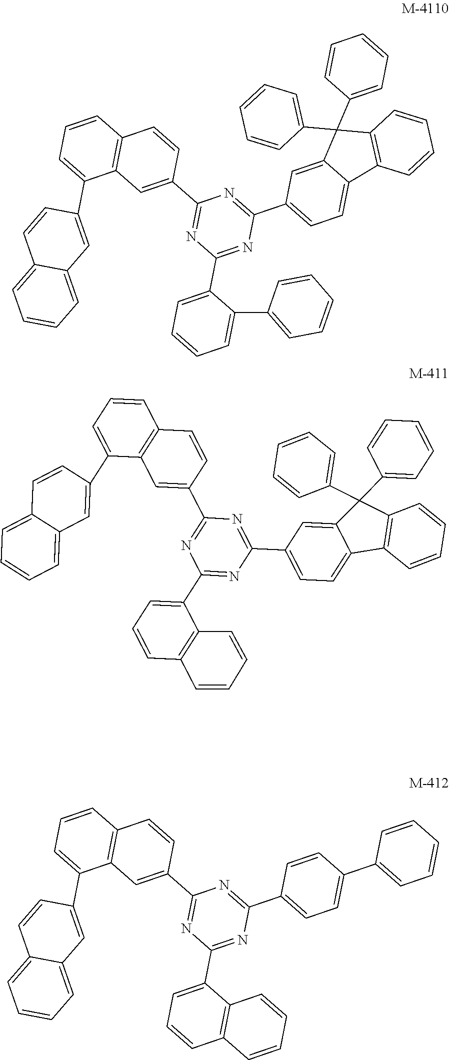 [SMILES: c1ccc(-c2ccccc2-c2nc(-c3ccc4c(c3)C(c3ccccc3)(c3ccccc3)c3ccccc3-4)nc(-c3ccc4cccc(-c5ccc6ccccc6c5)c4c3)n2)cc1 ; c1ccc(C2(c3ccccc3)c3ccccc3-c3ccc(-c4nc(-c5ccc6cccc(-c7ccc8ccccc8c7)c6c5)nc(-c5cccc6ccccc56)n4)cc32)cc1 ; c1ccc(-c2ccc(-c3nc(-c4ccc5cccc(-c6ccc7ccccc7c6)c5c4)nc(-c4cccc5ccccc45)n3)cc2)cc1]

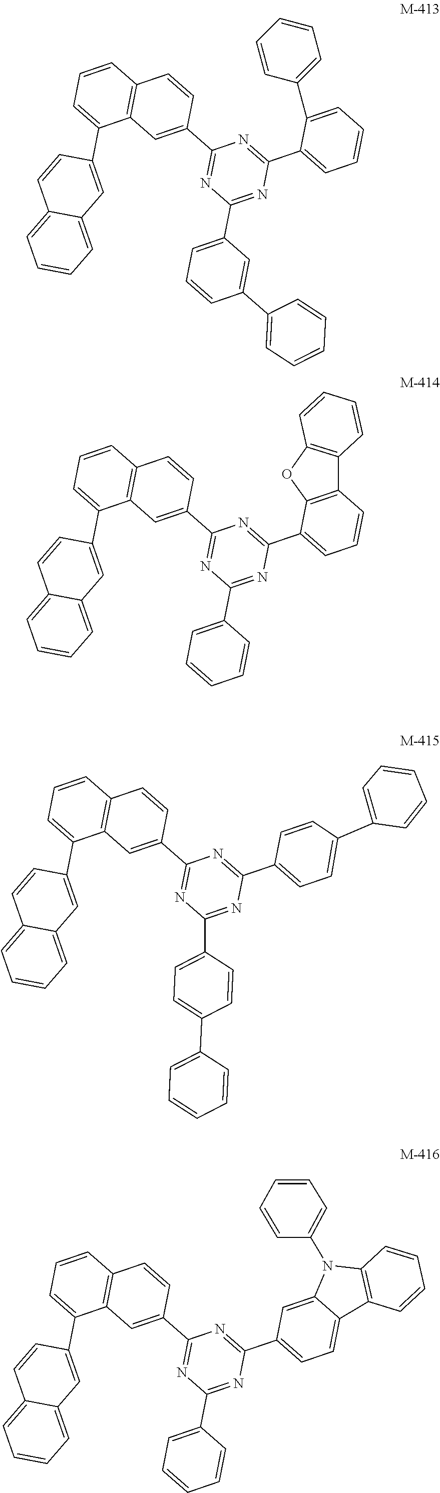

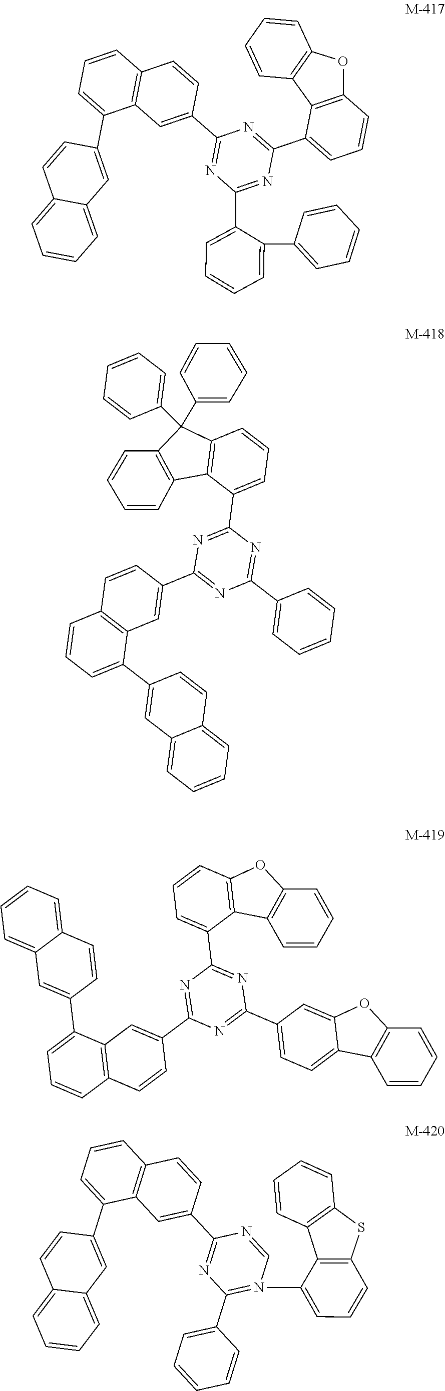

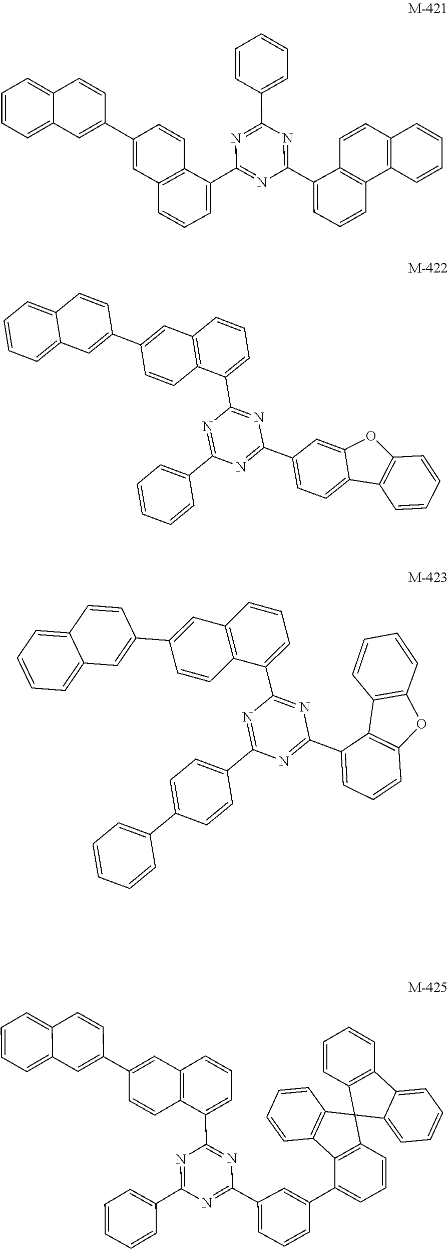 [SMILES: c1ccc(-c2nc(-c3cccc4cc(-c5ccc6ccccc6c5)ccc34)nc(-c3cccc4c3ccc3ccccc34)n2)cc1 ; c1ccc(-c2nc(-c3ccc4c(c3)oc3ccccc34)nc(-c3cccc4cc(-c5ccc6ccccc6c5)ccc34)n2)cc1 ; c1ccc(-c2ccc(-c3nc(-c4cccc5cc(-c6ccc7ccccc7c6)ccc45)nc(-c4cccc5oc6ccccc6c45)n3)cc2)cc1 ; c1ccc(-c2nc(-c3cccc(-c4cccc5c4-c4ccccc4C54c5ccccc5-c5ccccc54)c3)nc(-c3cccc4cc(-c5ccc6ccccc6c5)ccc34)n2)cc1]

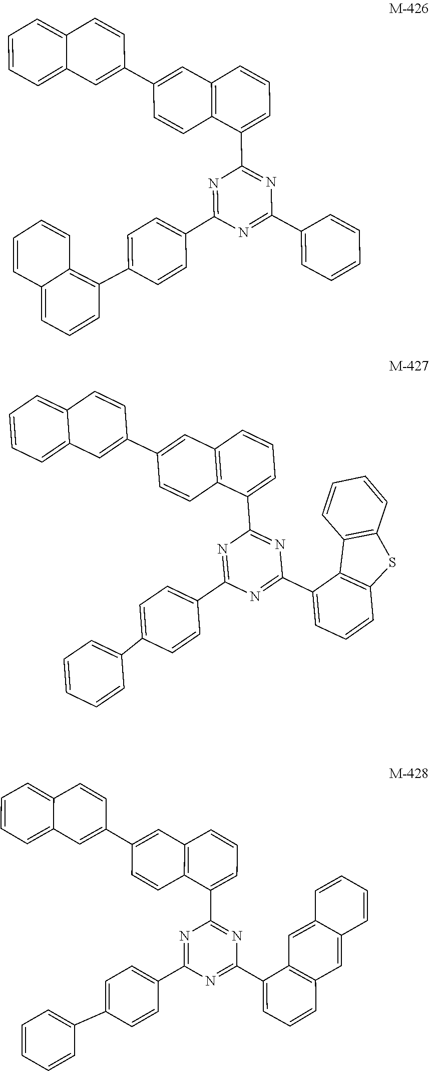 [SMILES: c1ccc(-c2nc(-c3ccc(-c4cccc5ccccc45)cc3)nc(-c3cccc4cc(-c5ccc6ccccc6c5)ccc34)n2)cc1 ; c1ccc(-c2ccc(-c3nc(-c4cccc5cc(-c6ccc7ccccc7c6)ccc45)nc(-c4cccc5sc6ccccc6c45)n3)cc2)cc1 ; c1ccc(-c2ccc(-c3nc(-c4cccc5cc(-c6ccc7ccccc7c6)ccc45)nc(-c4cccc5cc6ccccc6cc45)n3)cc2)cc1]

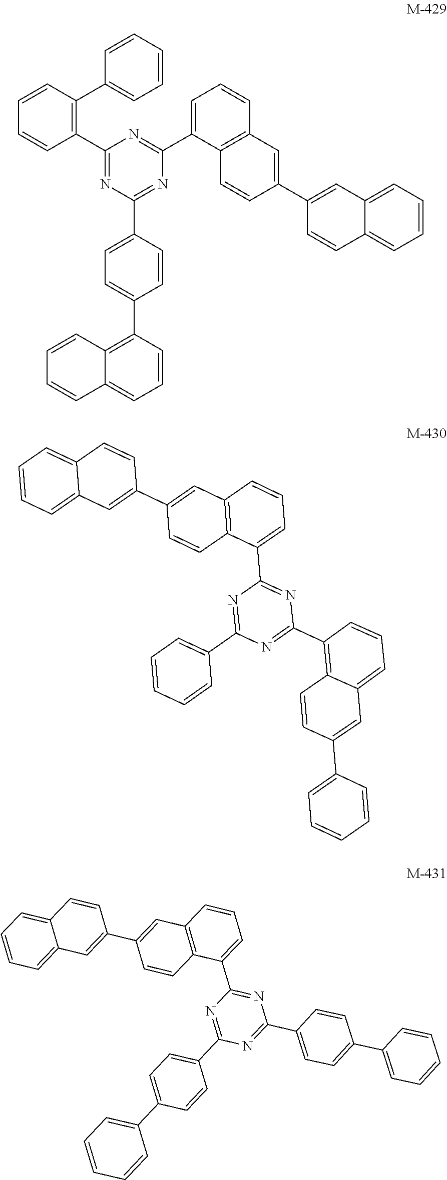 [SMILES: c1ccc(-c2ccccc2-c2nc(-c3ccc(-c4cccc5ccccc45)cc3)nc(-c3cccc4cc(-c5ccc6ccccc6c5)ccc34)n2)cc1 ; c1ccc(-c2ccc3c(-c4nc(-c5ccccc5)nc(-c5cccc6cc(-c7ccc8ccccc8c7)ccc56)n4)cccc3c2)cc1 ; c1ccc(-c2ccc(-c3nc(-c4ccc(-c5ccccc5)cc4)nc(-c4cccc5cc(-c6ccc7ccccc7c6)ccc45)n3)cc2)cc1]

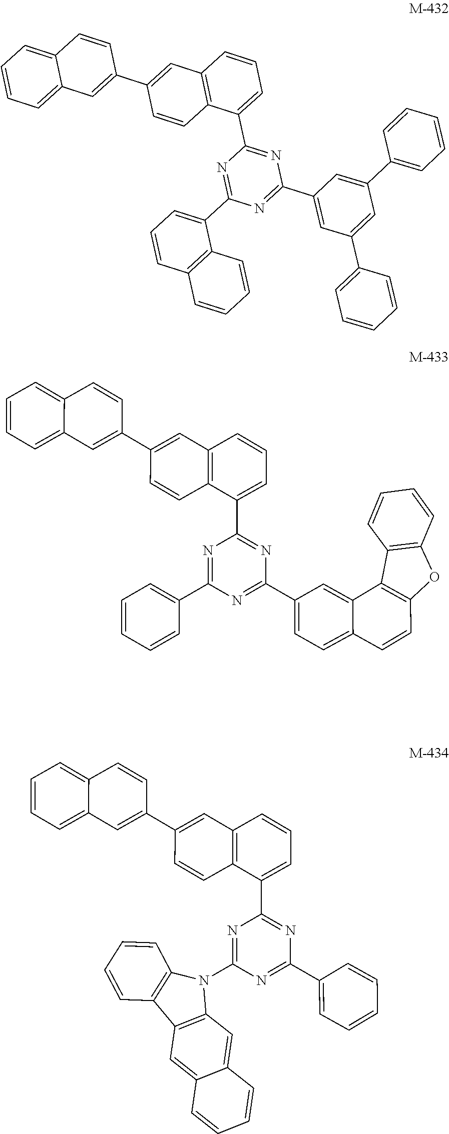 [SMILES: c1ccc(-c2cc(-c3ccccc3)cc(-c3nc(-c4cccc5ccccc45)nc(-c4cccc5cc(-c6ccc7ccccc7c6)ccc45)n3)c2)cc1 ; c1ccc(-c2nc(-c3ccc4ccc5oc6ccccc6c5c4c3)nc(-c3cccc4cc(-c5ccc6ccccc6c5)ccc34)n2)cc1 ; c1ccc(-c2nc(-c3cccc4cc(-c5ccc6ccccc6c5)ccc34)nc(-n3c4ccccc4c4cc5ccccc5cc43)n2)cc1]

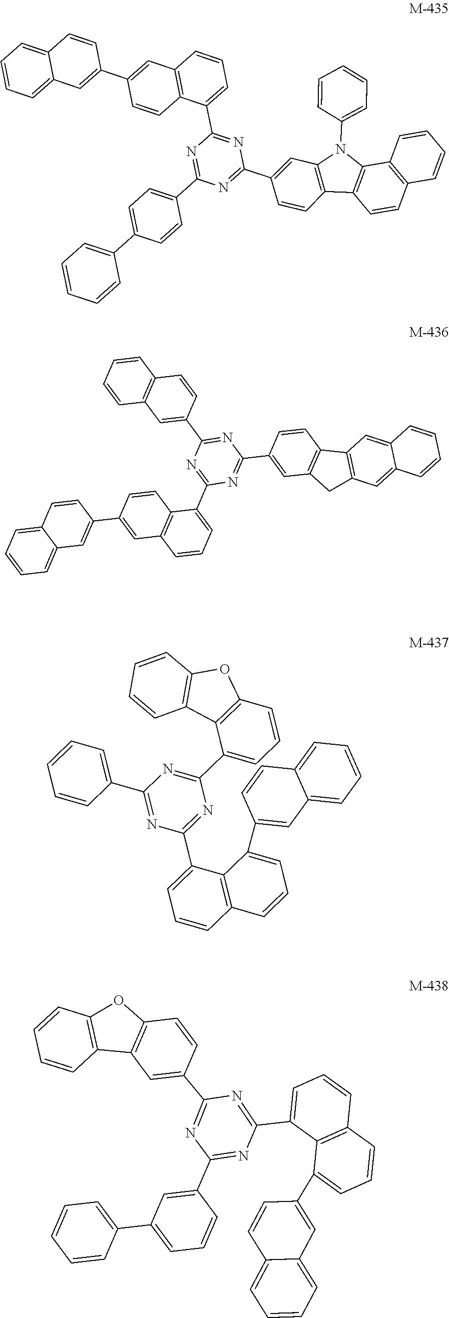 [SMILES: c1ccc(-c2ccc(-c3nc(-c4ccc5c6ccc7ccccc7c6n(-c6ccccc6)c5c4)nc(-c4cccc5cc(-c6ccc7ccccc7c6)ccc45)n3)cc2)cc1 ; c1ccc2cc(-c3ccc4c(-c5nc(-c6ccc7c(c6)Cc6cc8ccccc8cc6-7)nc(-c6ccc7ccccc7c6)n5)cccc4c3)ccc2c1 ; c1ccc(-c2nc(-c3cccc4cccc(-c5ccc6ccccc6c5)c34)nc(-c3cccc4oc5ccccc5c34)n2)cc1 ; c1ccc(-c2cccc(-c3nc(-c4ccc5oc6ccccc6c5c4)nc(-c4cccc5cccc(-c6ccc7ccccc7c6)c45)n3)c2)cc1]

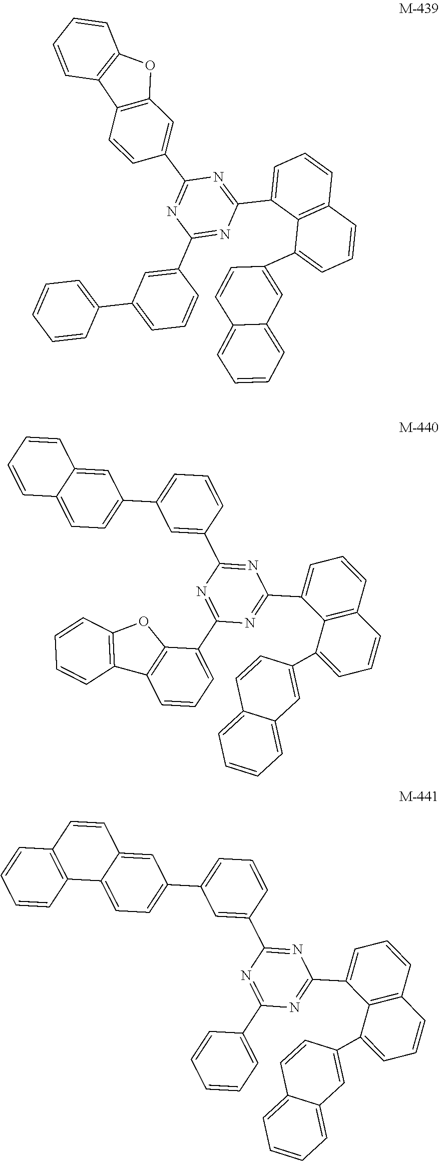 [SMILES: c1ccc(-c2cccc(-c3nc(-c4ccc5c(c4)oc4ccccc45)nc(-c4cccc5cccc(-c6ccc7ccccc7c6)c45)n3)c2)cc1 ; c1cc(-c2ccc3ccccc3c2)cc(-c2nc(-c3cccc4c3oc3ccccc34)nc(-c3cccc4cccc(-c5ccc6ccccc6c5)c34)n2)c1 ; c1ccc(-c2nc(-c3cccc(-c4ccc5c(ccc6ccccc65)c4)c3)nc(-c3cccc4cccc(-c5ccc6ccccc6c5)c34)n2)cc1]

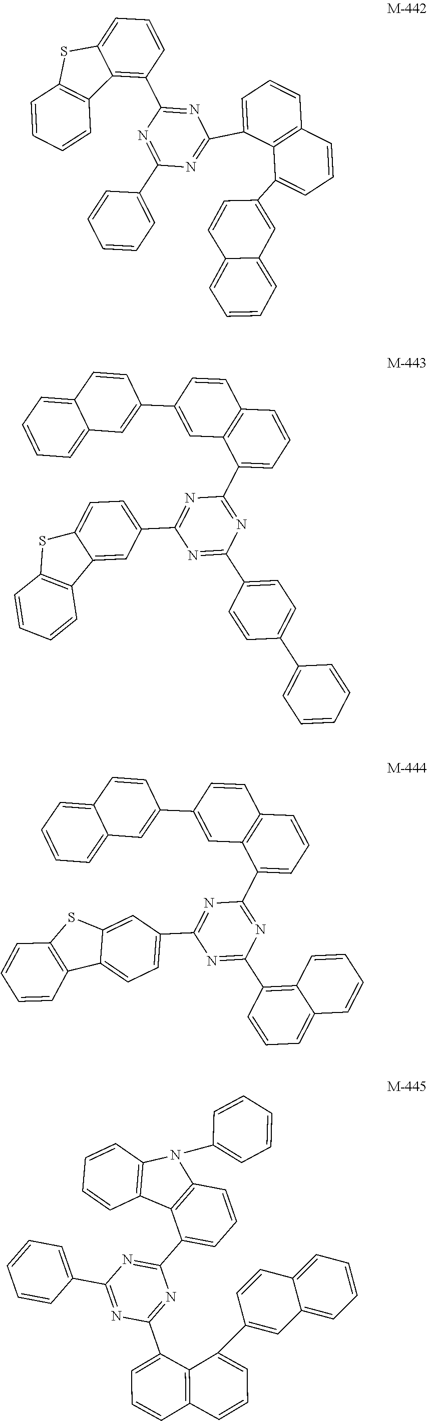 [SMILES: c1ccc(-c2nc(-c3cccc4cccc(-c5ccc6ccccc6c5)c34)nc(-c3cccc4sc5ccccc5c34)n2)cc1 ; c1ccc(-c2ccc(-c3nc(-c4ccc5sc6ccccc6c5c4)nc(-c4cccc5ccc(-c6ccc7ccccc7c6)cc45)n3)cc2)cc1 ; c1ccc2cc(-c3ccc4cccc(-c5nc(-c6ccc7c(c6)sc6ccccc67)nc(-c6cccc7ccccc67)n5)c4c3)ccc2c1 ; c1ccc(-c2nc(-c3cccc4cccc(-c5ccc6ccccc6c5)c34)nc(-c3cccc4c3c3ccccc3n4-c3ccccc3)n2)cc1]

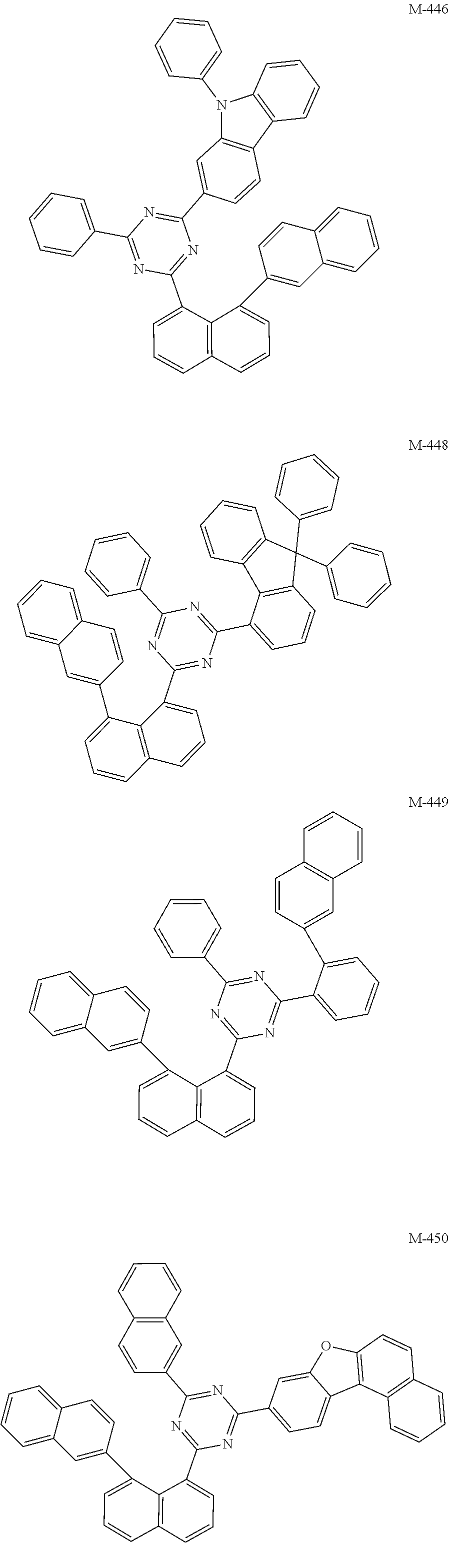 [SMILES: c1ccc(-c2nc(-c3ccc4c5ccccc5n(-c5ccccc5)c4c3)nc(-c3cccc4cccc(-c5ccc6ccccc6c5)c34)n2)cc1 ; c1ccc(-c2nc(-c3cccc4c3-c3ccccc3C4(c3ccccc3)c3ccccc3)nc(-c3cccc4cccc(-c5ccc6ccccc6c5)c34)n2)cc1 ; c1ccc(-c2nc(-c3ccccc3-c3ccc4ccccc4c3)nc(-c3cccc4cccc(-c5ccc6ccccc6c5)c34)n2)cc1 ; c1ccc2cc(-c3nc(-c4ccc5c(c4)oc4ccc6ccccc6c45)nc(-c4cccc5cccc(-c6ccc7ccccc7c6)c45)n3)ccc2c1]

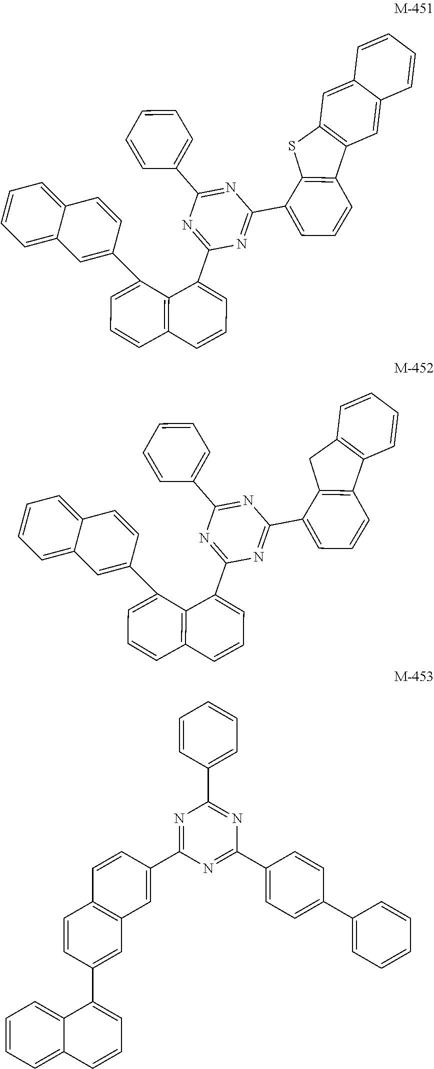

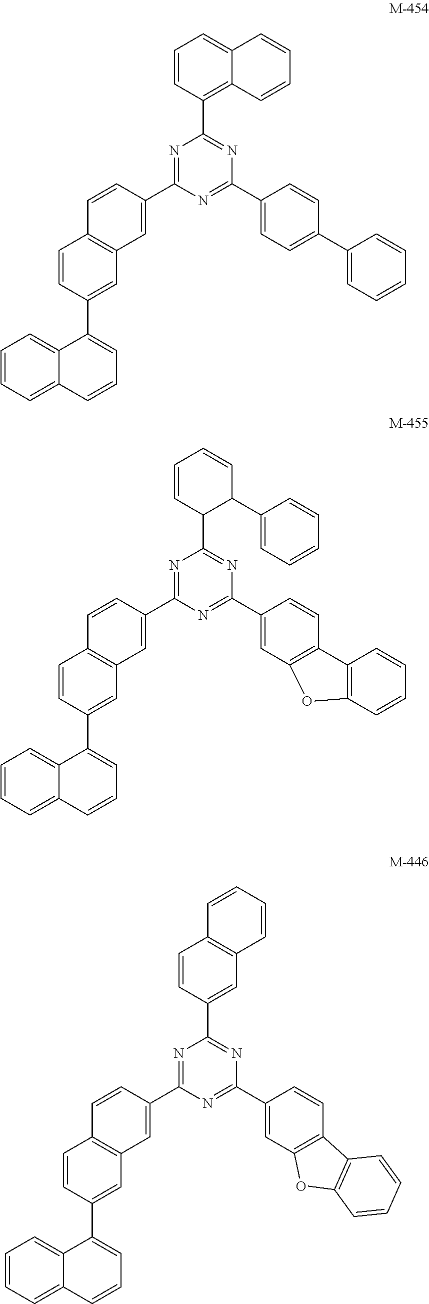 [SMILES: c1ccc(-c2ccc(-c3nc(-c4ccc5ccc(-c6cccc7ccccc67)cc5c4)nc(-c4cccc5ccccc45)n3)cc2)cc1 ; C1=CC(c2ccccc2)C(c2nc(-c3ccc4ccc(-c5cccc6ccccc56)cc4c3)nc(-c3ccc4c(c3)oc3ccccc34)n2)C=C1 ; c1ccc2cc(-c3nc(-c4ccc5ccc(-c6cccc7ccccc67)cc5c4)nc(-c4ccc5c(c4)oc4ccccc45)n3)ccc2c1]

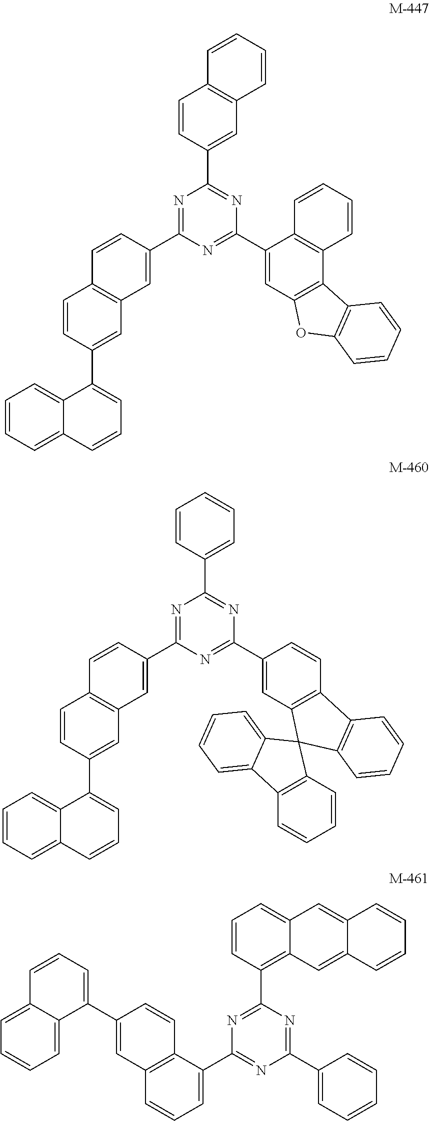 [SMILES: c1ccc2cc(-c3nc(-c4ccc5ccc(-c6cccc7ccccc67)cc5c4)nc(-c4cc5oc6ccccc6c5c5ccccc45)n3)ccc2c1 ; c1ccc(-c2nc(-c3ccc4c(c3)C3(c5ccccc5-c5ccccc53)c3ccccc3-4)nc(-c3ccc4ccc(-c5cccc6ccccc56)cc4c3)n2)cc1 ; c1ccc(-c2nc(-c3cccc4cc(-c5cccc6ccccc56)ccc34)nc(-c3cccc4cc5ccccc5cc34)n2)cc1]

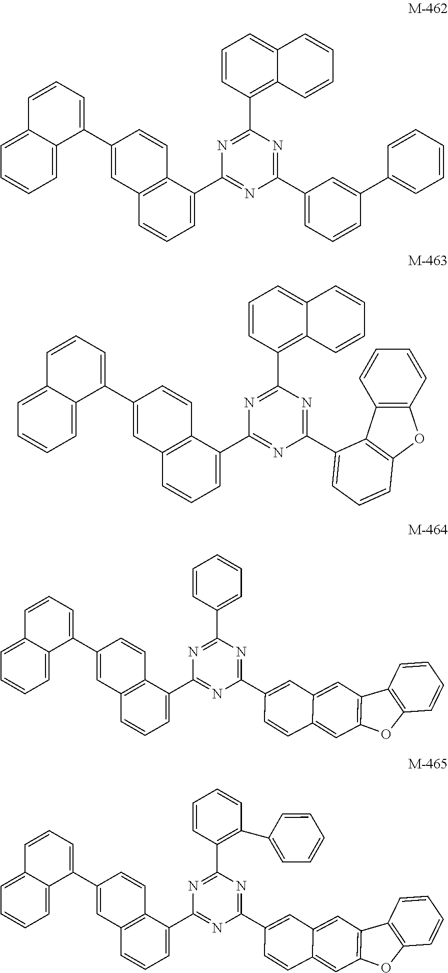 [SMILES: c1ccc(-c2cccc(-c3nc(-c4cccc5ccccc45)nc(-c4cccc5cc(-c6cccc7ccccc67)ccc45)n3)c2)cc1 ; c1ccc2c(-c3ccc4c(-c5nc(-c6cccc7ccccc67)nc(-c6cccc7oc8ccccc8c67)n5)cccc4c3)cccc2c1 ; c1ccc(-c2nc(-c3ccc4cc5oc6ccccc6c5cc4c3)nc(-c3cccc4cc(-c5cccc6ccccc56)ccc34)n2)cc1 ; c1ccc(-c2ccccc2-c2nc(-c3ccc4cc5oc6ccccc6c5cc4c3)nc(-c3cccc4cc(-c5cccc6ccccc56)ccc34)n2)cc1]

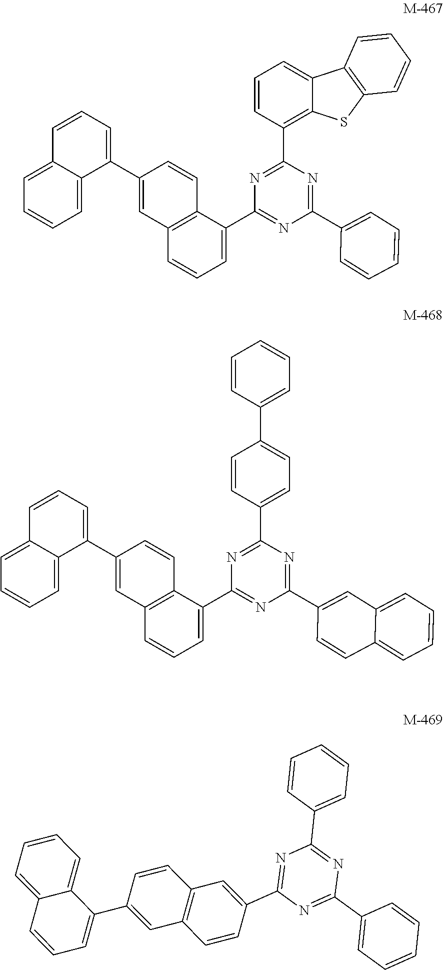 [SMILES: c1ccc(-c2nc(-c3cccc4cc(-c5cccc6ccccc56)ccc34)nc(-c3cccc4c3sc3ccccc34)n2)cc1 ; c1ccc(-c2ccc(-c3nc(-c4ccc5ccccc5c4)nc(-c4cccc5cc(-c6cccc7ccccc67)ccc45)n3)cc2)cc1 ; c1ccc(-c2nc(-c3ccccc3)nc(-c3ccc4cc(-c5cccc6ccccc56)ccc4c3)n2)cc1]

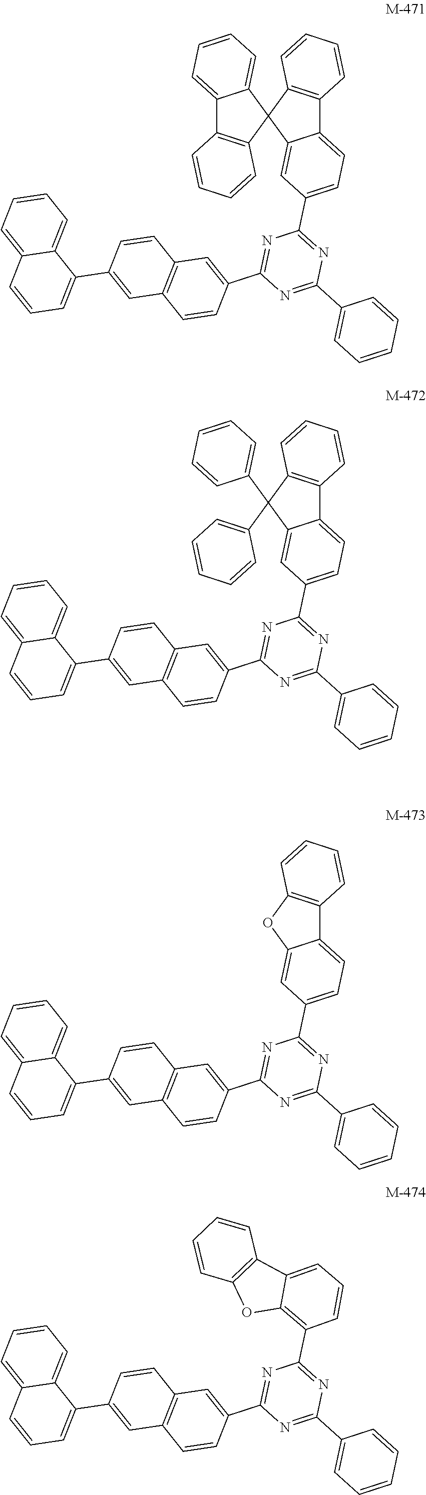 [SMILES: c1ccc(-c2nc(-c3ccc4c(c3)C3(c5ccccc5-c5ccccc53)c3ccccc3-4)nc(-c3ccc4cc(-c5cccc6ccccc56)ccc4c3)n2)cc1 ; c1ccc(-c2nc(-c3ccc4c(c3)C(c3ccccc3)(c3ccccc3)c3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccccc34)n2)cc1 ; c1ccc(-c2nc(-c3ccc4cc(-c5cccc6ccccc56)ccc4c3)nc(-c3cccc4c3oc3ccccc34)n2)cc1]

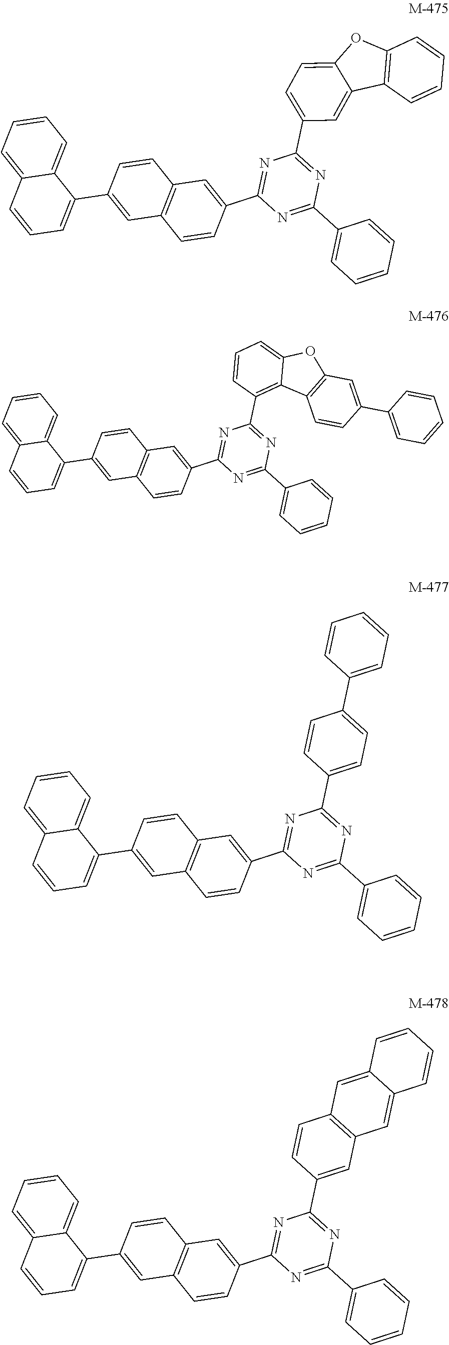 [SMILES: c1ccc(-c2nc(-c3ccc4cc(-c5cccc6ccccc56)ccc4c3)nc(-c3ccc4oc5ccccc5c4c3)n2)cc1 ; c1ccc(-c2ccc3c(c2)oc2cccc(-c4nc(-c5ccccc5)nc(-c5ccc6cc(-c7cccc8ccccc78)ccc6c5)n4)c23)cc1 ; c1ccc(-c2ccc(-c3nc(-c4ccccc4)nc(-c4ccc5cc(-c6cccc7ccccc67)ccc5c4)n3)cc2)cc1 ; c1ccc(-c2nc(-c3ccc4cc(-c5cccc6ccccc56)ccc4c3)nc(-c3ccc4cc5ccccc5cc4c3)n2)cc1]

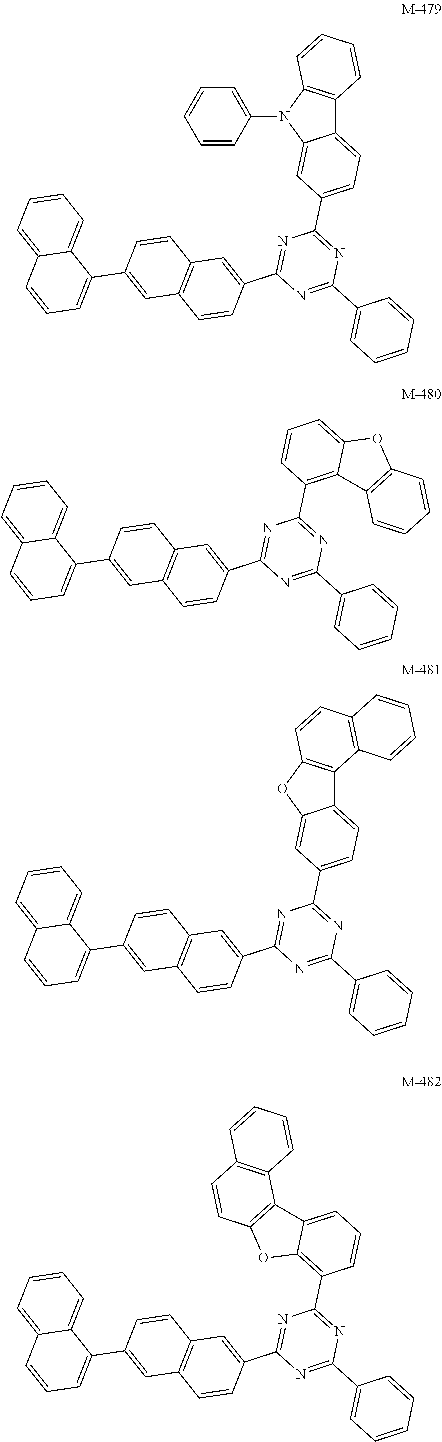 [SMILES: c1ccc(-c2nc(-c3ccc4cc(-c5cccc6ccccc56)ccc4c3)nc(-c3ccc4c5ccccc5n(-c5ccccc5)c4c3)n2)cc1 ; c1ccc(-c2nc(-c3ccc4cc(-c5cccc6ccccc56)ccc4c3)nc(-c3cccc4oc5ccccc5c34)n2)cc1 ; c1ccc(-c2nc(-c3ccc4cc(-c5cccc6ccccc56)ccc4c3)nc(-c3ccc4c(c3)oc3ccc5ccccc5c34)n2)cc1 ; c1ccc(-c2nc(-c3ccc4cc(-c5cccc6ccccc56)ccc4c3)nc(-c3cccc4c3oc3ccc5ccccc5c34)n2)cc1]

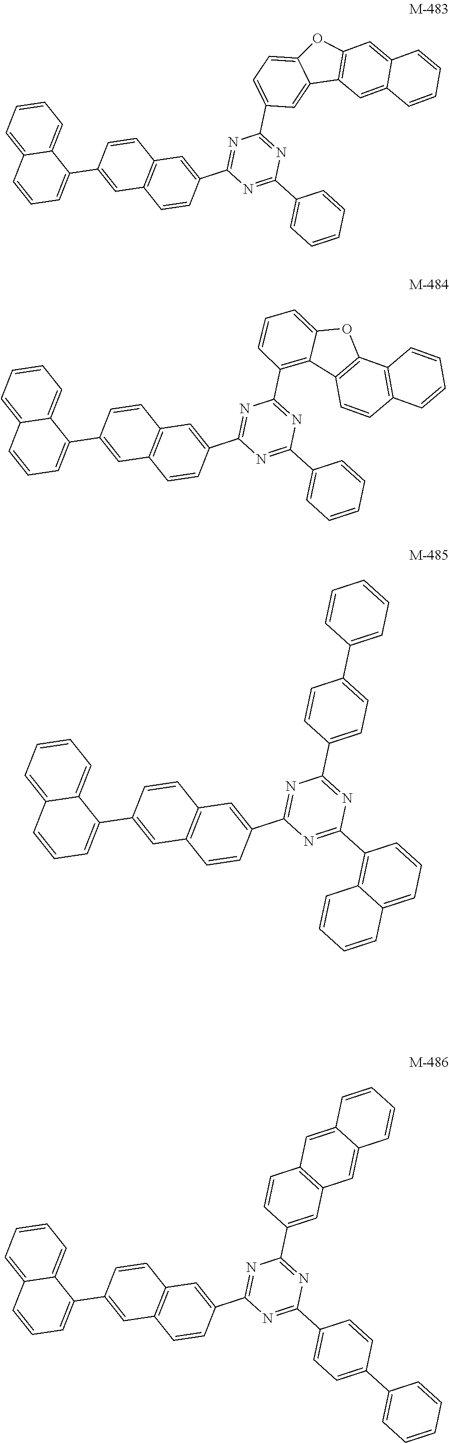 [SMILES: c1ccc(-c2nc(-c3ccc4cc(-c5cccc6ccccc56)ccc4c3)nc(-c3ccc4oc5cc6ccccc6cc5c4c3)n2)cc1 ; c1ccc(-c2nc(-c3ccc4cc(-c5cccc6ccccc56)ccc4c3)nc(-c3cccc4oc5c6ccccc6ccc5c34)n2)cc1 ; c1ccc(-c2ccc(-c3nc(-c4ccc5cc(-c6cccc7ccccc67)ccc5c4)nc(-c4cccc5ccccc45)n3)cc2)cc1 ; c1ccc(-c2ccc(-c3nc(-c4ccc5cc(-c6cccc7ccccc67)ccc5c4)nc(-c4ccc5cc6ccccc6cc5c4)n3)cc2)cc1]

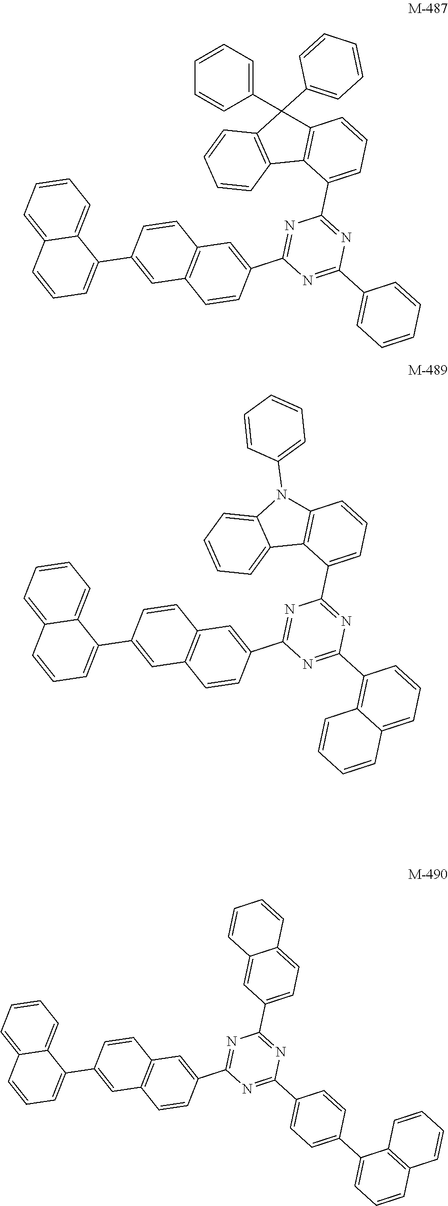 [SMILES: c1ccc(-c2nc(-c3ccc4cc(-c5cccc6ccccc56)ccc4c3)nc(-c3cccc4c3-c3ccccc3C4(c3ccccc3)c3ccccc3)n2)cc1 ; c1ccc(-n2c3ccccc3c3c(-c4nc(-c5ccc6cc(-c7cccc8ccccc78)ccc6c5)nc(-c5cccc6ccccc56)n4)cccc32)cc1 ; c1ccc2cc(-c3nc(-c4ccc(-c5cccc6ccccc56)cc4)nc(-c4ccc5cc(-c6cccc7ccccc67)ccc5c4)n3)ccc2c1]

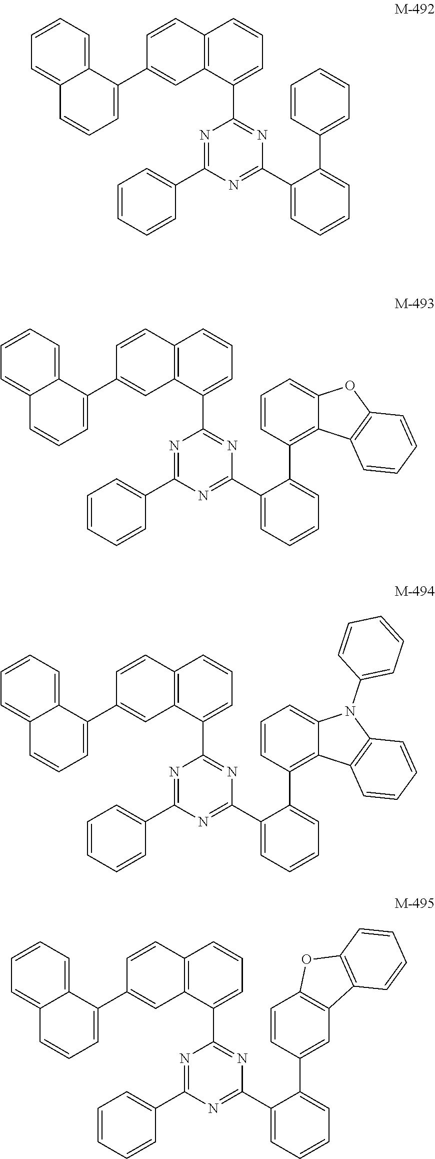 [SMILES: c1ccc(-c2nc(-c3ccccc3-c3ccccc3)nc(-c3cccc4ccc(-c5cccc6ccccc56)cc34)n2)cc1 ; c1ccc(-c2nc(-c3ccccc3-c3cccc4oc5ccccc5c34)nc(-c3cccc4ccc(-c5cccc6ccccc56)cc34)n2)cc1 ; c1ccc(-c2nc(-c3ccccc3-c3cccc4c3c3ccccc3n4-c3ccccc3)nc(-c3cccc4ccc(-c5cccc6ccccc56)cc34)n2)cc1 ; c1ccc(-c2nc(-c3ccccc3-c3ccc4oc5ccccc5c4c3)nc(-c3cccc4ccc(-c5cccc6ccccc56)cc34)n2)cc1]

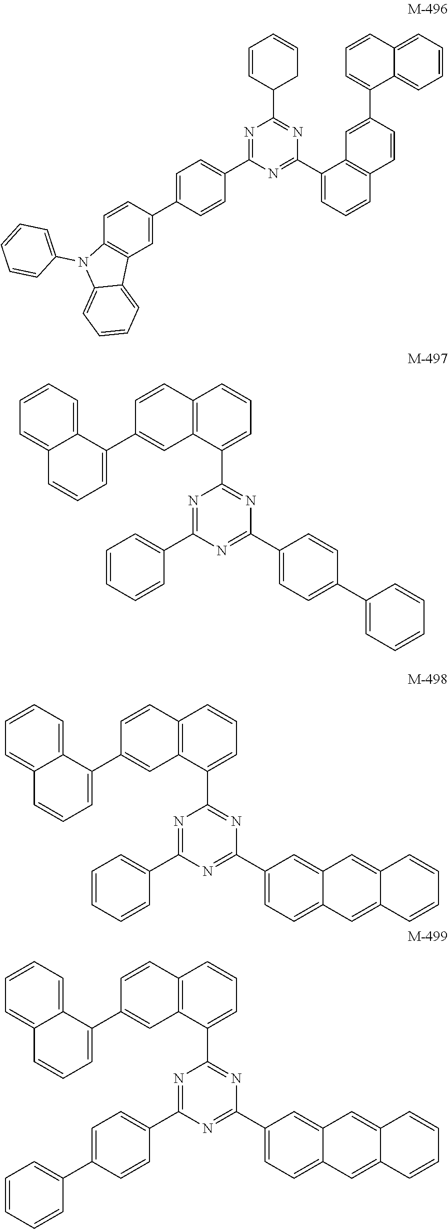 [SMILES: C1=CCC(c2nc(-c3ccc(-c4ccc5c(c4)c4ccccc4n5-c4ccccc4)cc3)nc(-c3cccc4ccc(-c5cccc6ccccc56)cc34)n2)C=C1 ; c1ccc(-c2ccc(-c3nc(-c4ccccc4)nc(-c4cccc5ccc(-c6cccc7ccccc67)cc45)n3)cc2)cc1 ; c1ccc(-c2nc(-c3ccc4cc5ccccc5cc4c3)nc(-c3cccc4ccc(-c5cccc6ccccc56)cc34)n2)cc1 ; c1ccc(-c2ccc(-c3nc(-c4ccc5cc6ccccc6cc5c4)nc(-c4cccc5ccc(-c6cccc7ccccc67)cc45)n3)cc2)cc1]

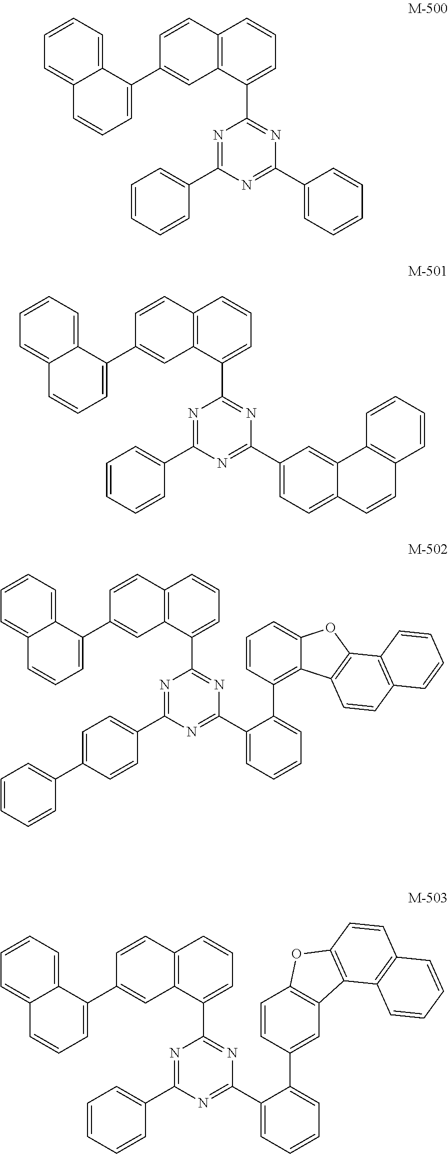 [SMILES: c1ccc(-c2nc(-c3ccccc3)nc(-c3cccc4ccc(-c5cccc6ccccc56)cc34)n2)cc1 ; c1ccc(-c2nc(-c3ccc4ccc5ccccc5c4c3)nc(-c3cccc4ccc(-c5cccc6ccccc56)cc34)n2)cc1 ; c1ccc(-c2ccc(-c3nc(-c4ccccc4-c4cccc5oc6c7ccccc7ccc6c45)nc(-c4cccc5ccc(-c6cccc7ccccc67)cc45)n3)cc2)cc1 ; c1ccc(-c2nc(-c3ccccc3-c3ccc4oc5ccc6ccccc6c5c4c3)nc(-c3cccc4ccc(-c5cccc6ccccc56)cc34)n2)cc1]

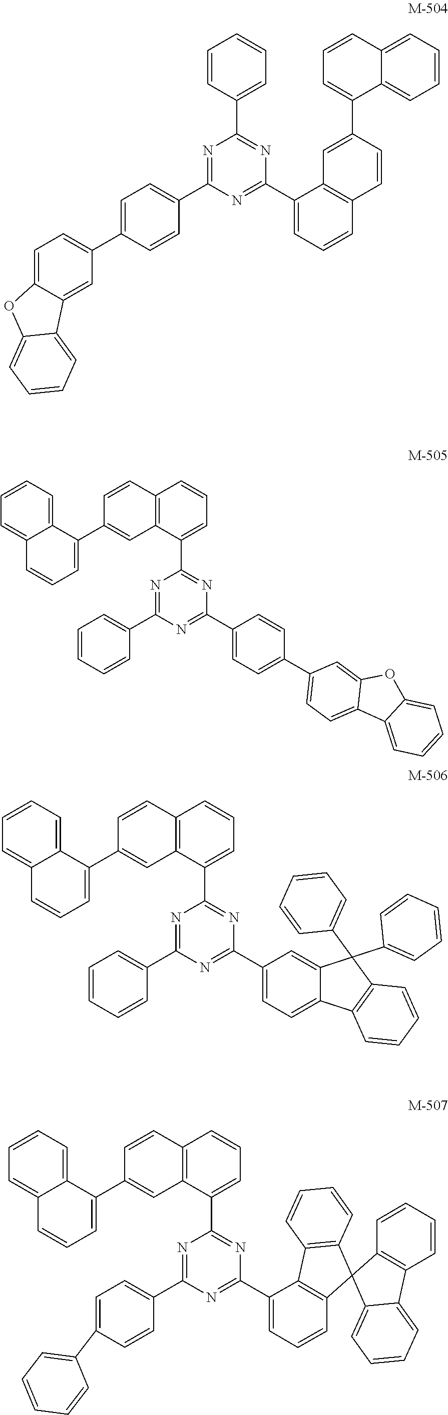 [SMILES: c1ccc(-c2nc(-c3ccc(-c4ccc5oc6ccccc6c5c4)cc3)nc(-c3cccc4ccc(-c5cccc6ccccc56)cc34)n2)cc1 ; c1ccc(-c2nc(-c3ccc(-c4ccc5c(c4)oc4ccccc45)cc3)nc(-c3cccc4ccc(-c5cccc6ccccc56)cc34)n2)cc1 ; c1ccc(-c2nc(-c3ccc4c(c3)C(c3ccccc3)(c3ccccc3)c3ccccc3-4)nc(-c3cccc4ccc(-c5cccc6ccccc56)cc34)n2)cc1 ; c1ccc(-c2ccc(-c3nc(-c4cccc5c4-c4ccccc4C54c5ccccc5-c5ccccc54)nc(-c4cccc5ccc(-c6cccc7ccccc67)cc45)n3)cc2)cc1]

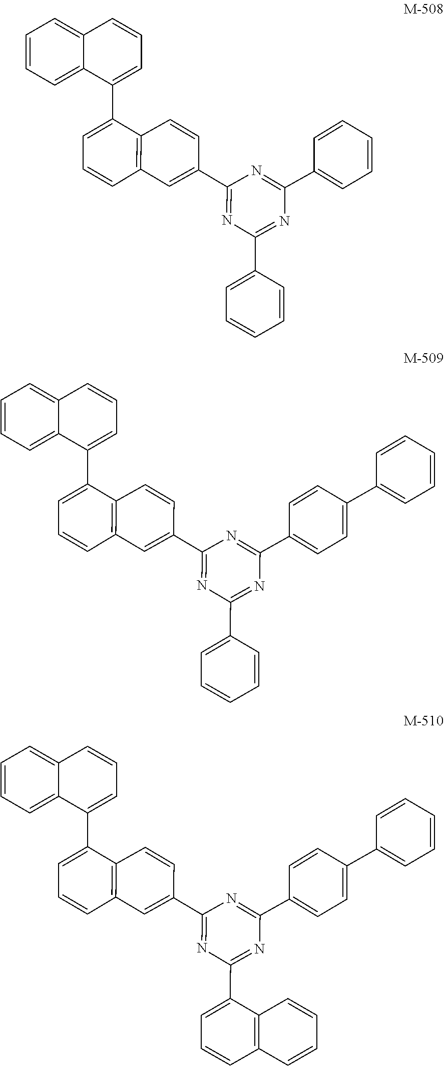 [SMILES: c1ccc(-c2nc(-c3ccccc3)nc(-c3ccc4c(-c5cccc6ccccc56)cccc4c3)n2)cc1 ; c1ccc(-c2ccc(-c3nc(-c4ccccc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4cccc5ccccc45)n3)cc2)cc1]

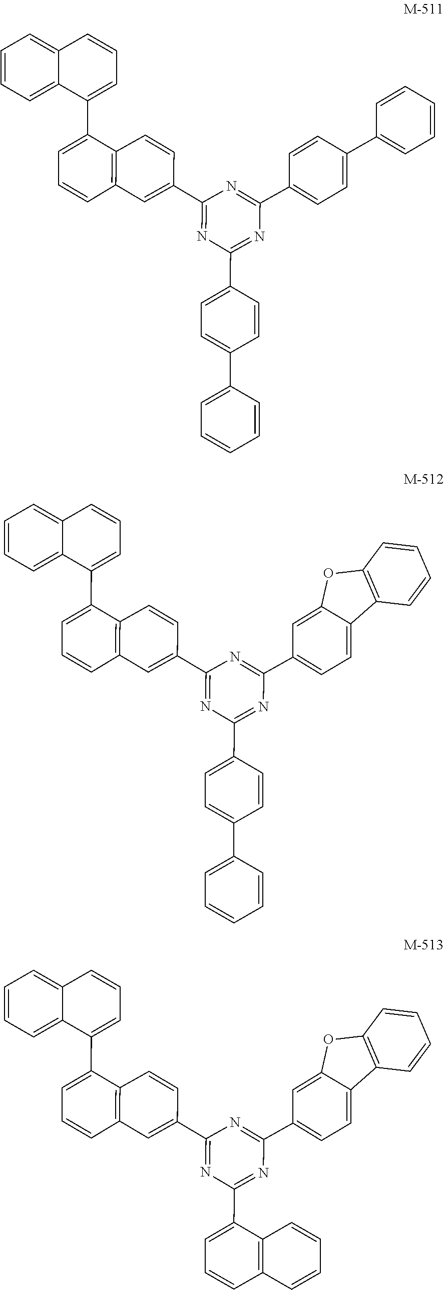 [SMILES: c1ccc(-c2ccc(-c3nc(-c4ccc(-c5ccccc5)cc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4ccc5c(c4)oc4ccccc45)n3)cc2)cc1 ; c1ccc2c(-c3nc(-c4ccc5c(-c6cccc7ccccc67)cccc5c4)nc(-c4ccc5c(c4)oc4ccccc45)n3)cccc2c1]

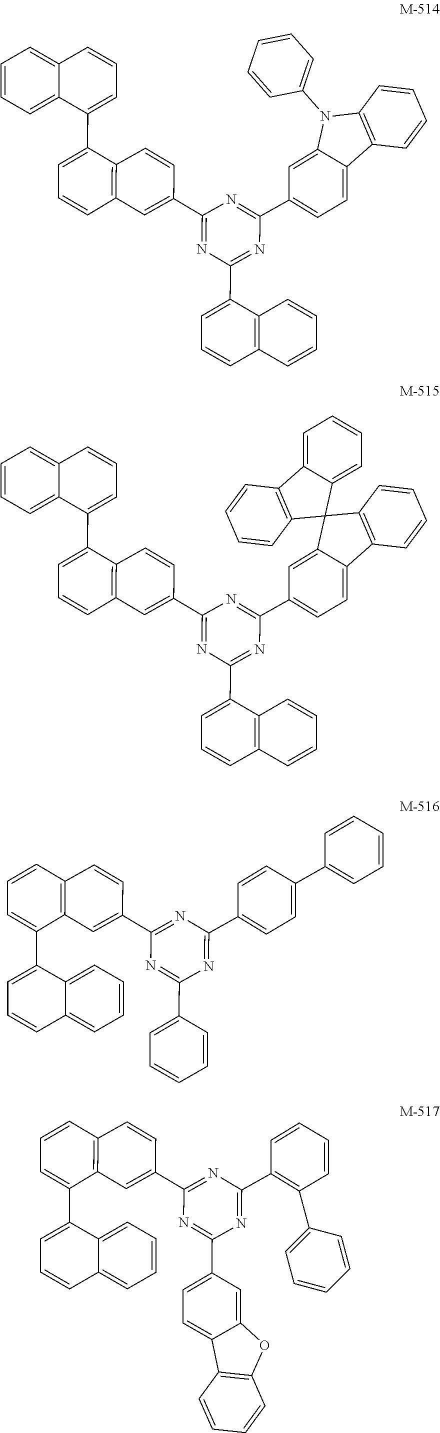 [SMILES: c1ccc(-n2c3ccccc3c3ccc(-c4nc(-c5ccc6c(-c7cccc8ccccc78)cccc6c5)nc(-c5cccc6ccccc56)n4)cc32)cc1 ; c1ccc2c(c1)-c1ccccc1C21c2ccccc2-c2ccc(-c3nc(-c4ccc5c(-c6cccc7ccccc67)cccc5c4)nc(-c4cccc5ccccc45)n3)cc21 ; c1ccc(-c2ccc(-c3nc(-c4ccccc4)nc(-c4ccc5cccc(-c6cccc7ccccc67)c5c4)n3)cc2)cc1 ; c1ccc(-c2ccccc2-c2nc(-c3ccc4c(c3)oc3ccccc34)nc(-c3ccc4cccc(-c5cccc6ccccc56)c4c3)n2)cc1]

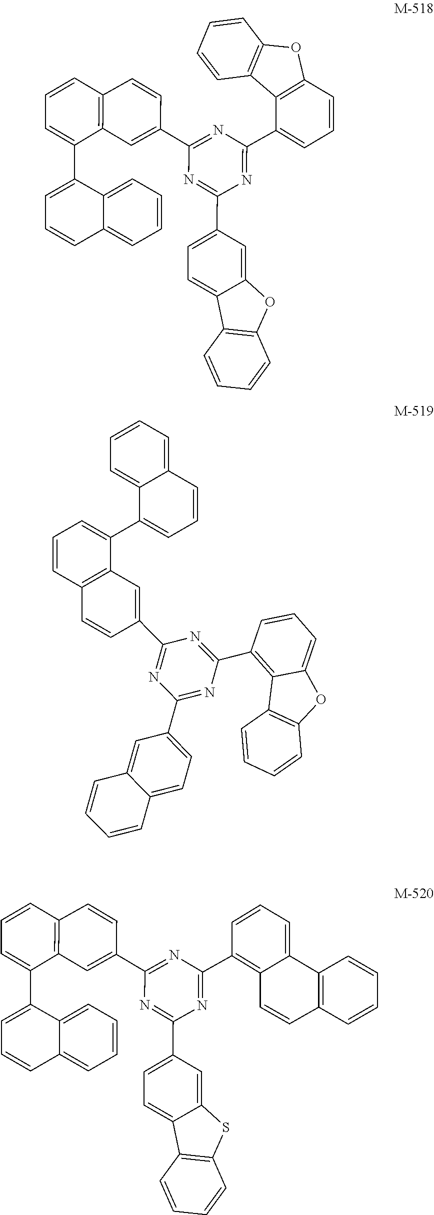 [SMILES: c1ccc2c(-c3cccc4ccc(-c5nc(-c6ccc7c(c6)oc6ccccc67)nc(-c6cccc7oc8ccccc8c67)n5)cc34)cccc2c1 ; c1ccc2cc(-c3nc(-c4ccc5cccc(-c6cccc7ccccc67)c5c4)nc(-c4cccc5oc6ccccc6c45)n3)ccc2c1 ; c1ccc2c(-c3cccc4ccc(-c5nc(-c6ccc7c(c6)sc6ccccc67)nc(-c6cccc7c6ccc6ccccc67)n5)cc34)cccc2c1]

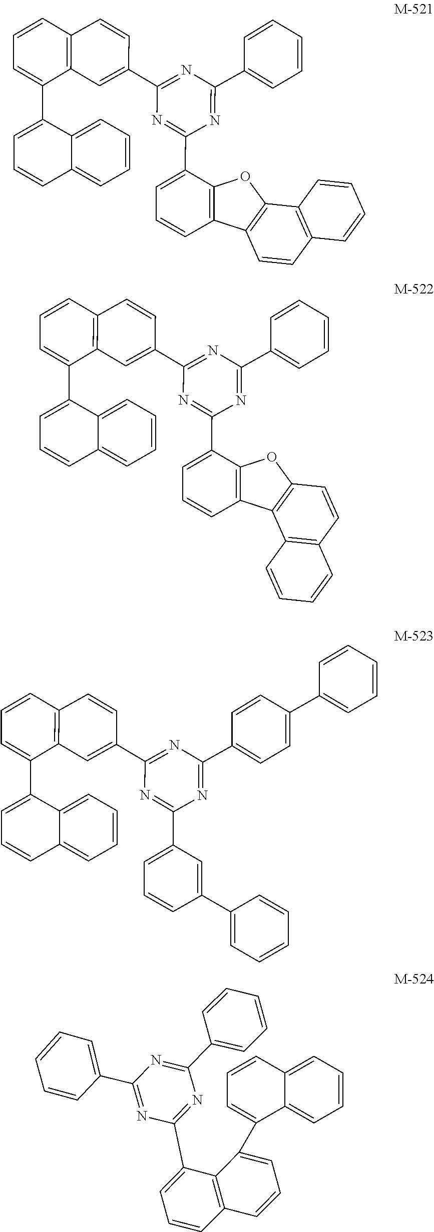

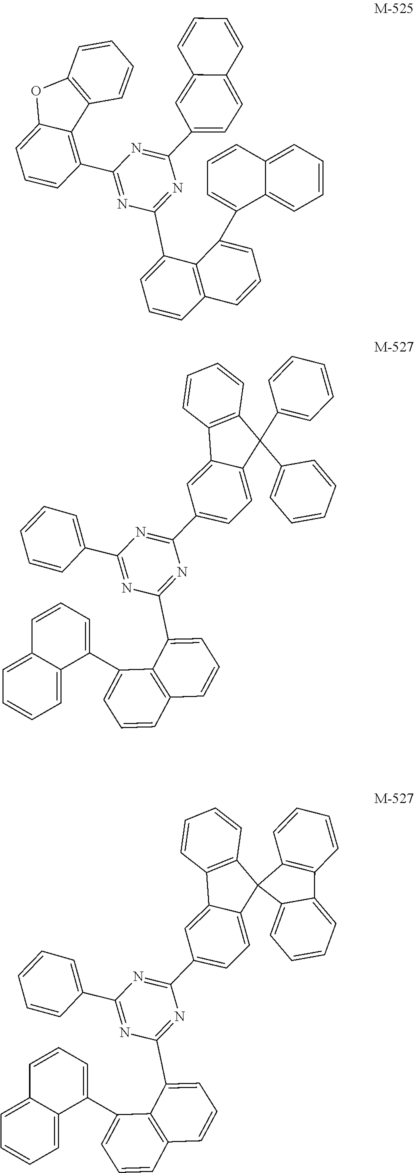 [SMILES: c1ccc2cc(-c3nc(-c4cccc5cccc(-c6cccc7ccccc67)c45)nc(-c4cccc5oc6ccccc6c45)n3)ccc2c1 ; c1ccc(-c2nc(-c3ccc4c(c3)-c3ccccc3C4(c3ccccc3)c3ccccc3)nc(-c3cccc4cccc(-c5cccc6ccccc56)c34)n2)cc1 ; c1ccc(-c2nc(-c3ccc4c(c3)-c3ccccc3C43c4ccccc4-c4ccccc43)nc(-c3cccc4cccc(-c5cccc6ccccc56)c34)n2)cc1]

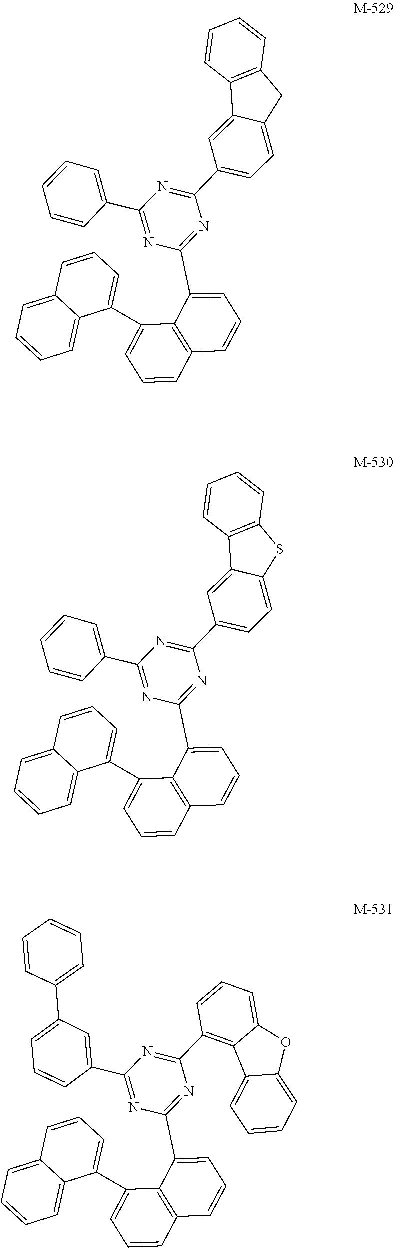 [SMILES: c1ccc(-c2nc(-c3ccc4c(c3)-c3ccccc3C4)nc(-c3cccc4cccc(-c5cccc6ccccc56)c34)n2)cc1 ; c1ccc(-c2nc(-c3ccc4sc5ccccc5c4c3)nc(-c3cccc4cccc(-c5cccc6ccccc56)c34)n2)cc1 ; c1ccc(-c2cccc(-c3nc(-c4cccc5cccc(-c6cccc7ccccc67)c45)nc(-c4cccc5oc6ccccc6c45)n3)c2)cc1]

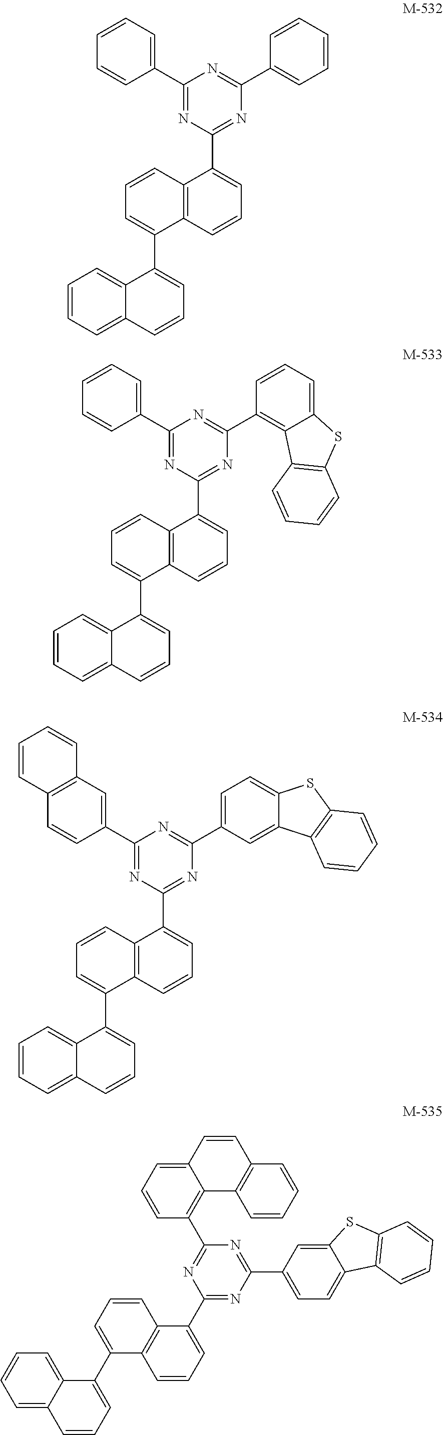

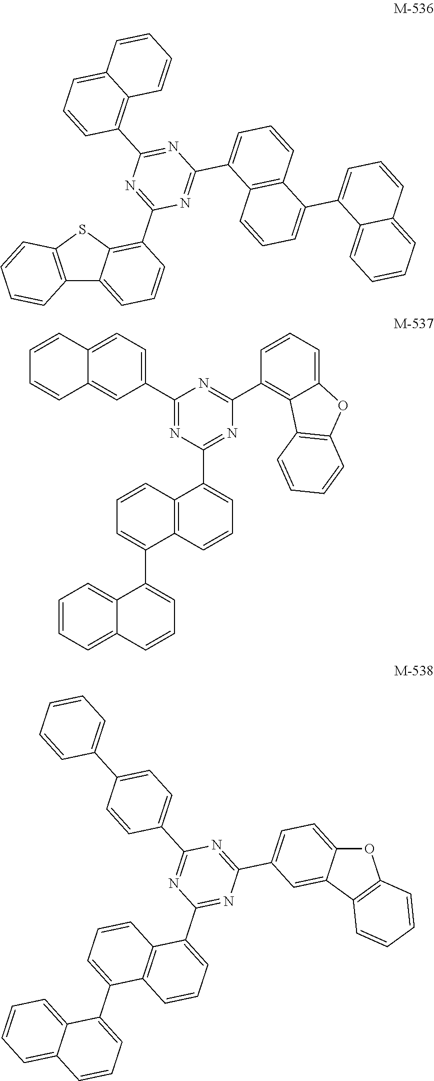

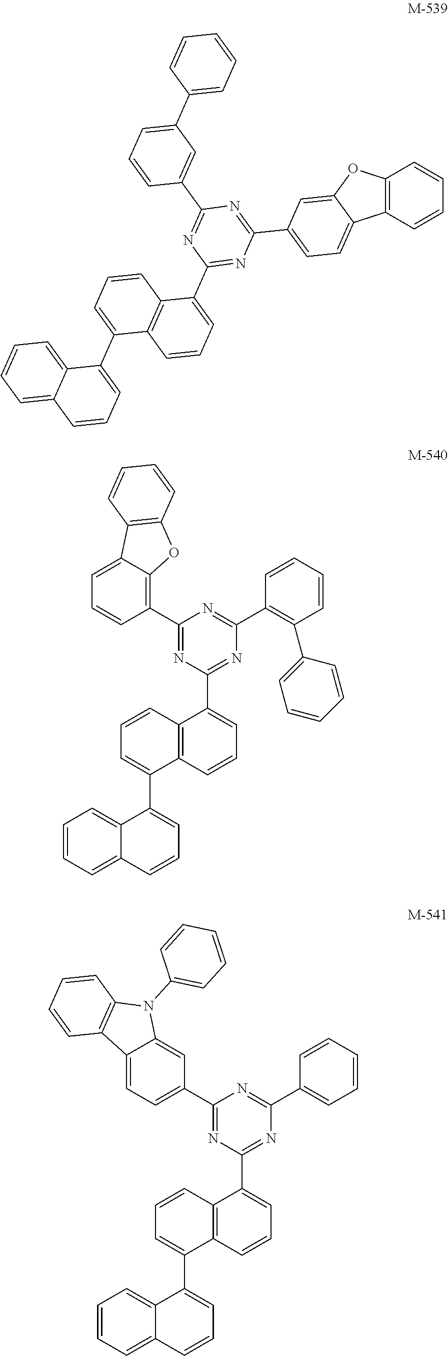 [SMILES: c1ccc(-c2cccc(-c3nc(-c4ccc5c(c4)oc4ccccc45)nc(-c4cccc5c(-c6cccc7ccccc67)cccc45)n3)c2)cc1 ; c1ccc(-c2ccccc2-c2nc(-c3cccc4c(-c5cccc6ccccc56)cccc34)nc(-c3cccc4c3oc3ccccc34)n2)cc1 ; c1ccc(-c2nc(-c3ccc4c5ccccc5n(-c5ccccc5)c4c3)nc(-c3cccc4c(-c5cccc6ccccc56)cccc34)n2)cc1]

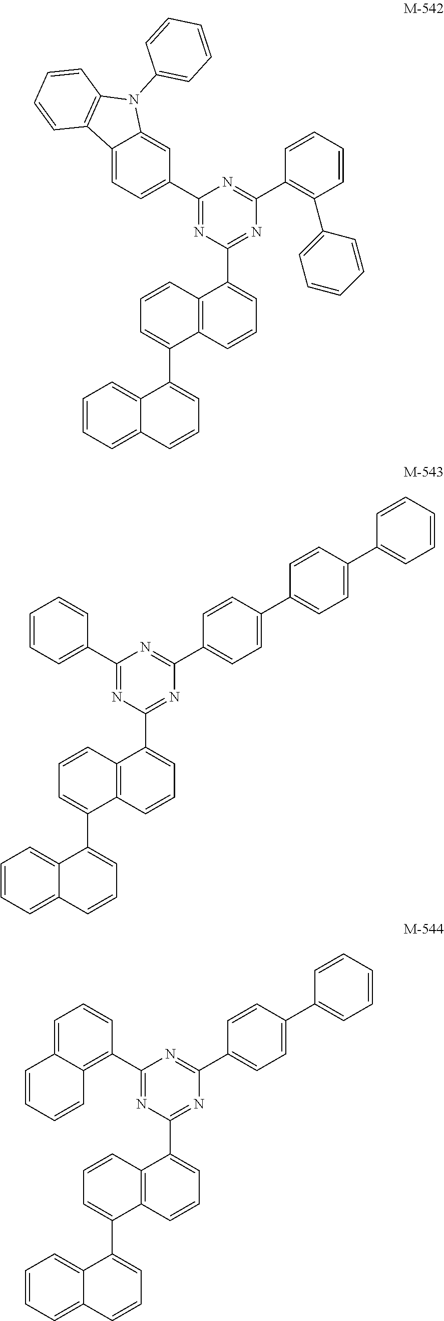 [SMILES: c1ccc(-c2ccccc2-c2nc(-c3ccc4c5ccccc5n(-c5ccccc5)c4c3)nc(-c3cccc4c(-c5cccc6ccccc56)cccc34)n2)cc1 ; c1ccc(-c2ccc(-c3ccc(-c4nc(-c5ccccc5)nc(-c5cccc6c(-c7cccc8ccccc78)cccc56)n4)cc3)cc2)cc1 ; c1ccc(-c2ccc(-c3nc(-c4cccc5ccccc45)nc(-c4cccc5c(-c6cccc7ccccc67)cccc45)n3)cc2)cc1]

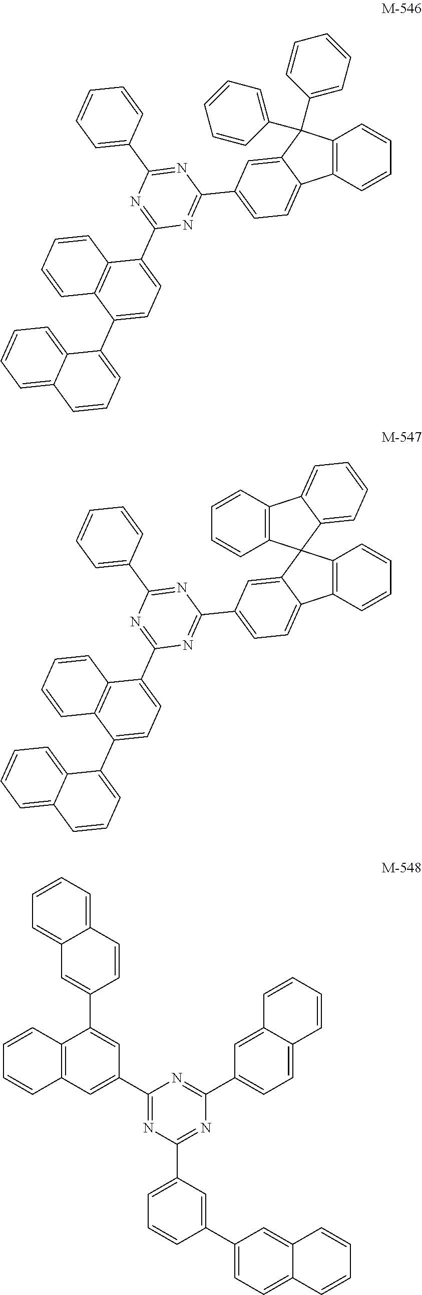 [SMILES: c1ccc(-c2nc(-c3ccc4c(c3)C(c3ccccc3)(c3ccccc3)c3ccccc3-4)nc(-c3ccc(-c4cccc5ccccc45)c4ccccc34)n2)cc1 ; c1ccc(-c2nc(-c3ccc4c(c3)C3(c5ccccc5-c5ccccc53)c3ccccc3-4)nc(-c3ccc(-c4cccc5ccccc45)c4ccccc34)n2)cc1 ; c1cc(-c2ccc3ccccc3c2)cc(-c2nc(-c3ccc4ccccc4c3)nc(-c3cc(-c4ccc5ccccc5c4)c4ccccc4c3)n2)c1]

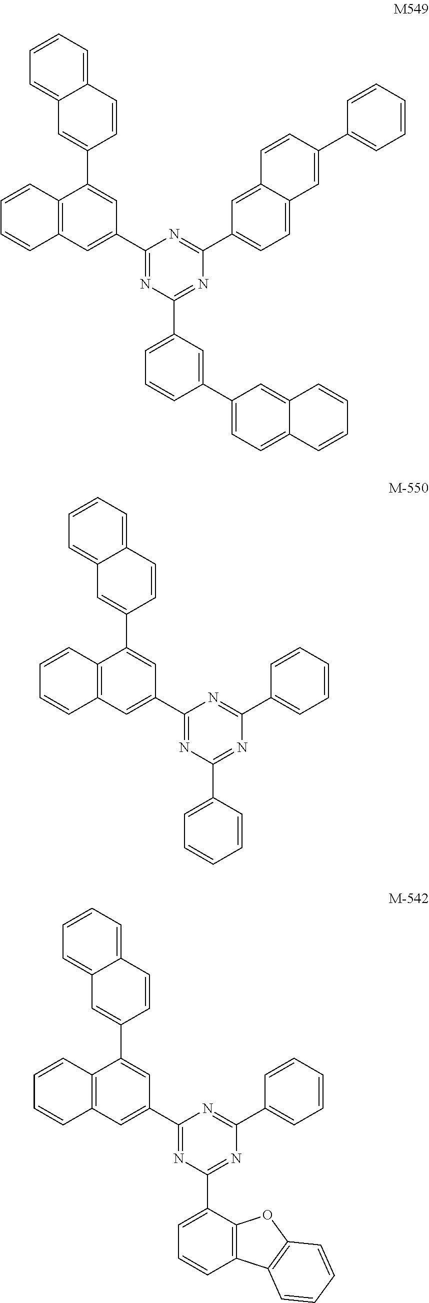 [SMILES: c1ccc(-c2ccc3cc(-c4nc(-c5cccc(-c6ccc7ccccc7c6)c5)nc(-c5cc(-c6ccc7ccccc7c6)c6ccccc6c5)n4)ccc3c2)cc1 ; c1ccc(-c2nc(-c3ccccc3)nc(-c3cc(-c4ccc5ccccc5c4)c4ccccc4c3)n2)cc1 ; c1ccc(-c2nc(-c3cc(-c4ccc5ccccc5c4)c4ccccc4c3)nc(-c3cccc4c3oc3ccccc34)n2)cc1]

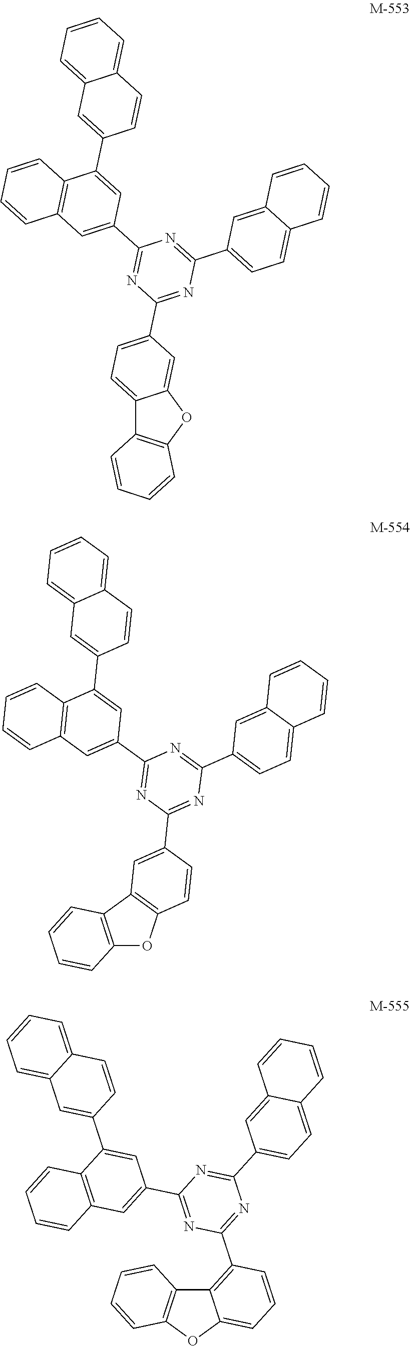 [SMILES: c1ccc2cc(-c3nc(-c4cc(-c5ccc6ccccc6c5)c5ccccc5c4)nc(-c4ccc5c(c4)oc4ccccc45)n3)ccc2c1 ; c1ccc2cc(-c3nc(-c4cc(-c5ccc6ccccc6c5)c5ccccc5c4)nc(-c4ccc5oc6ccccc6c5c4)n3)ccc2c1 ; c1ccc2cc(-c3nc(-c4cc(-c5ccc6ccccc6c5)c5ccccc5c4)nc(-c4cccc5oc6ccccc6c45)n3)ccc2c1]

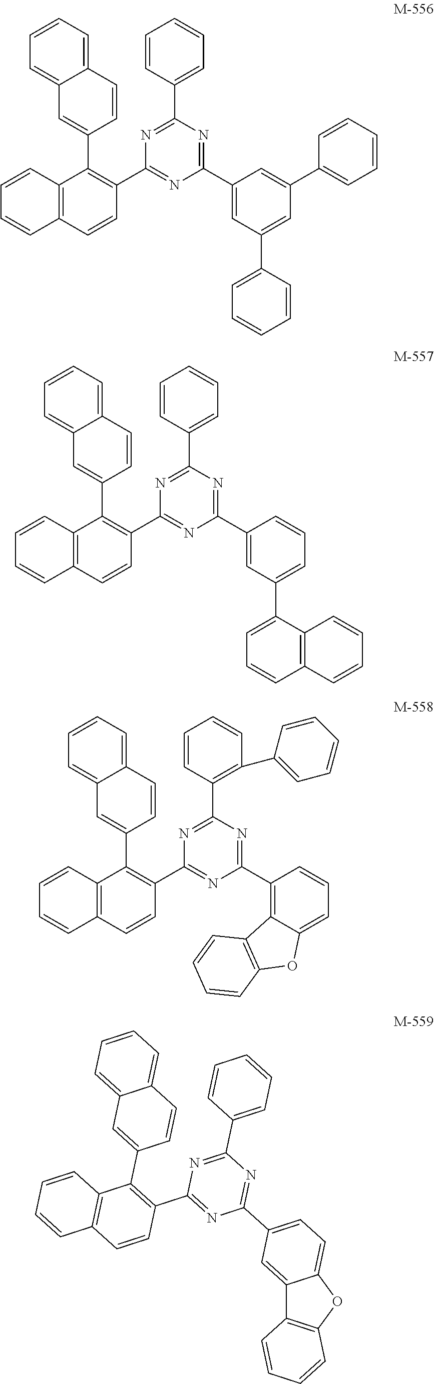

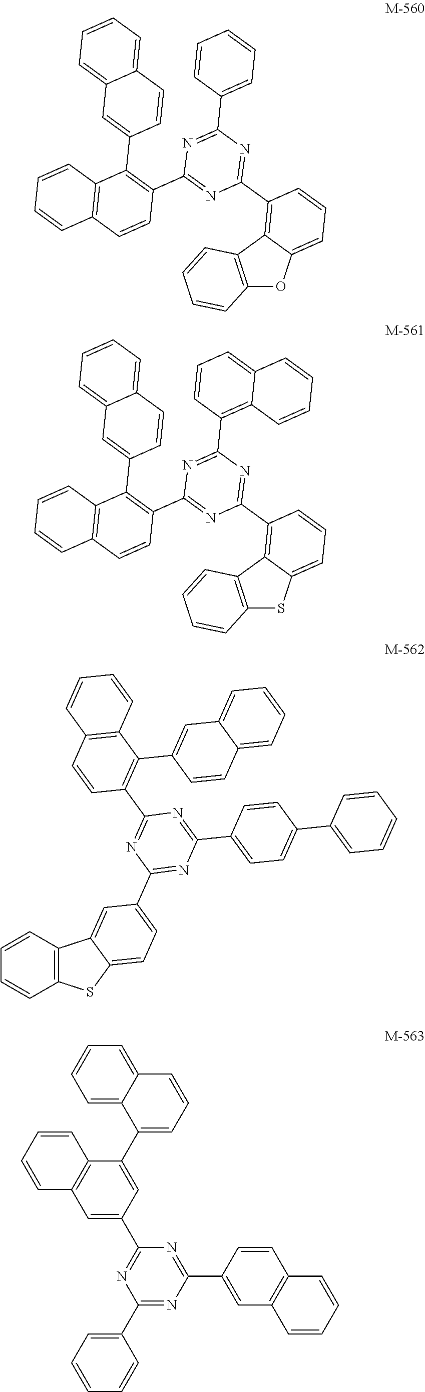

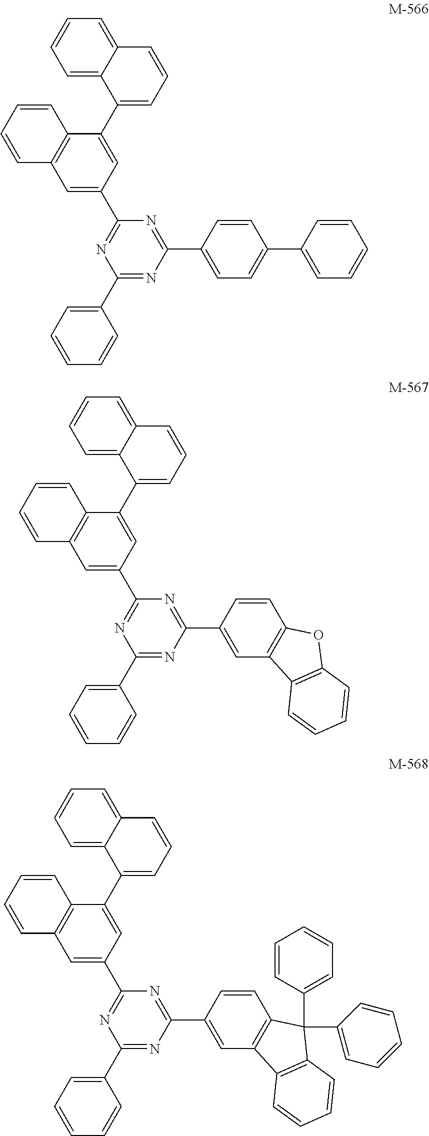

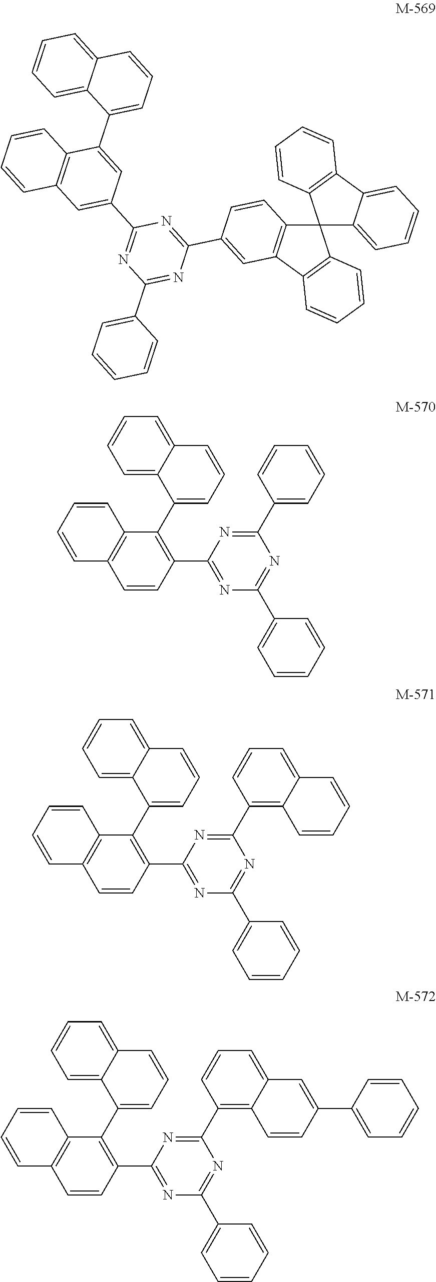

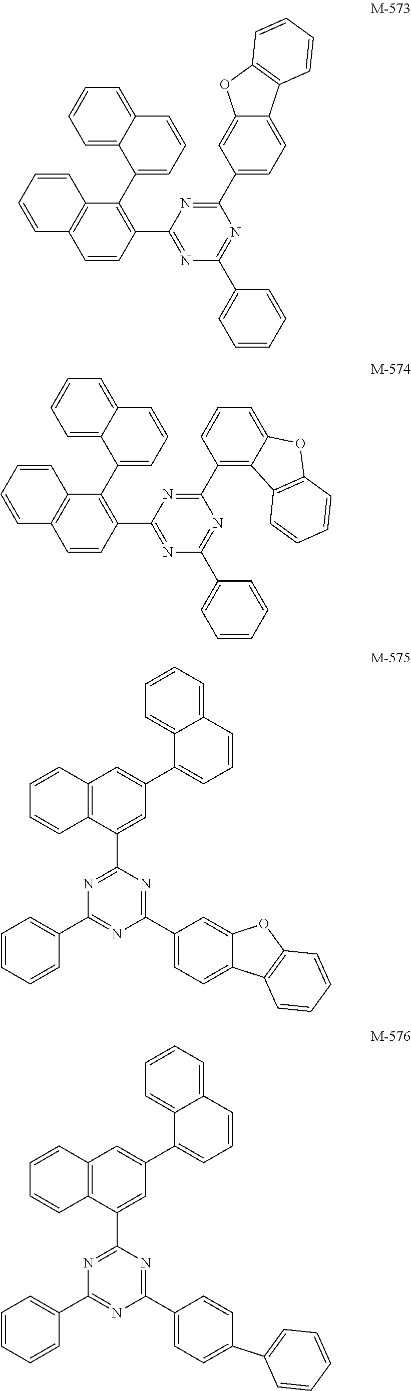 [SMILES: c1ccc(-c2nc(-c3ccc4c(c3)oc3ccccc34)nc(-c3ccc4ccccc4c3-c3cccc4ccccc34)n2)cc1 ; c1ccc(-c2nc(-c3ccc4ccccc4c3-c3cccc4ccccc34)nc(-c3cccc4oc5ccccc5c34)n2)cc1 ; c1ccc(-c2nc(-c3ccc4c(c3)oc3ccccc34)nc(-c3cc(-c4cccc5ccccc45)cc4ccccc34)n2)cc1 ; c1ccc(-c2ccc(-c3nc(-c4ccccc4)nc(-c4cc(-c5cccc6ccccc56)cc5ccccc45)n3)cc2)cc1]

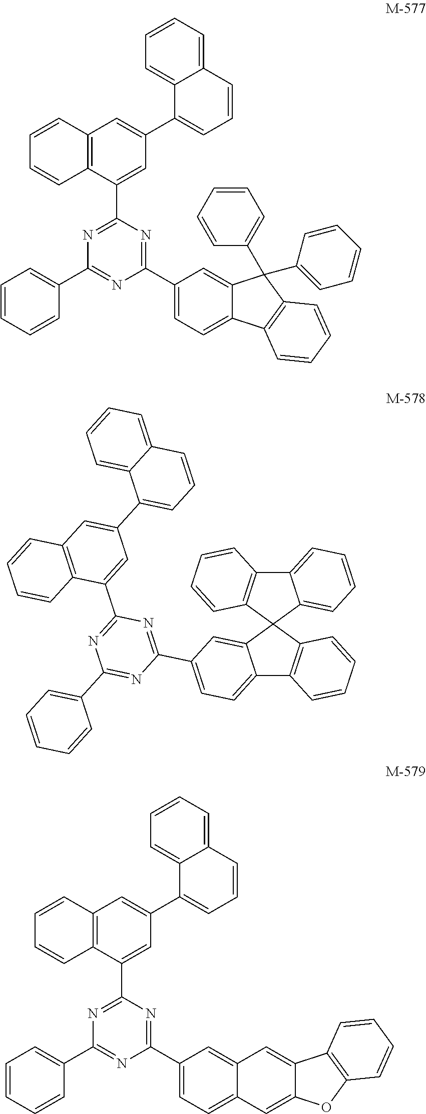 [SMILES: c1ccc(-c2nc(-c3ccc4c(c3)C(c3ccccc3)(c3ccccc3)c3ccccc3-4)nc(-c3cc(-c4cccc5ccccc45)cc4ccccc34)n2)cc1 ; c1ccc(-c2nc(-c3ccc4c(c3)C3(c5ccccc5-c5ccccc53)c3ccccc3-4)nc(-c3cc(-c4cccc5ccccc45)cc4ccccc34)n2)cc1 ; c1ccc(-c2nc(-c3ccc4cc5oc6ccccc6c5cc4c3)nc(-c3cc(-c4cccc5ccccc45)cc4ccccc34)n2)cc1]

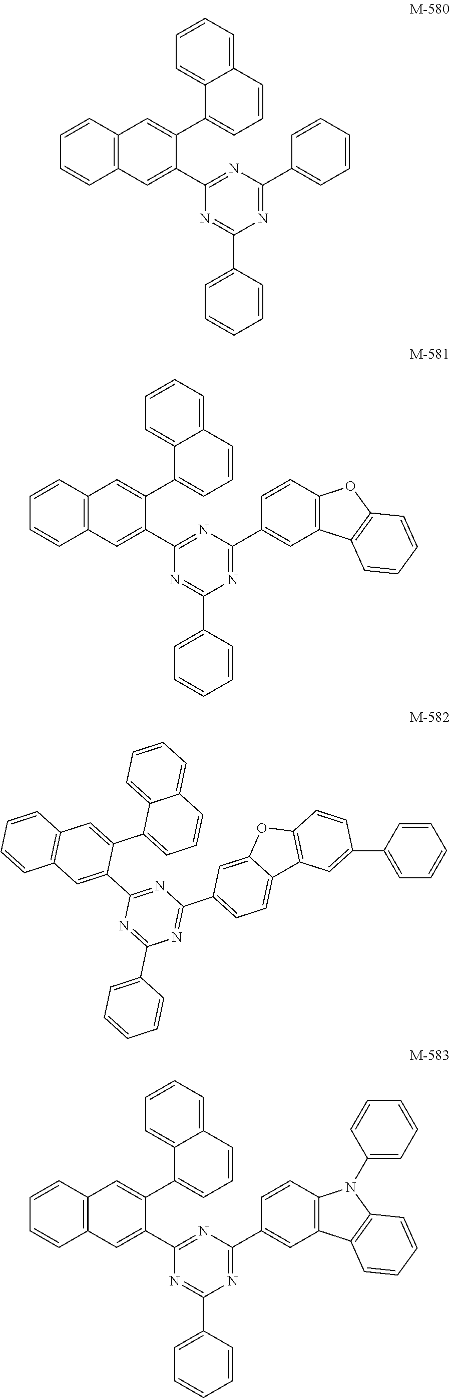 [SMILES: c1ccc(-c2nc(-c3ccccc3)nc(-c3cc4ccccc4cc3-c3cccc4ccccc34)n2)cc1 ; c1ccc(-c2nc(-c3ccc4oc5ccccc5c4c3)nc(-c3cc4ccccc4cc3-c3cccc4ccccc34)n2)cc1 ; c1ccc(-c2ccc3oc4cc(-c5nc(-c6ccccc6)nc(-c6cc7ccccc7cc6-c6cccc7ccccc67)n5)ccc4c3c2)cc1 ; c1ccc(-c2nc(-c3ccc4c(c3)c3ccccc3n4-c3ccccc3)nc(-c3cc4ccccc4cc3-c3cccc4ccccc34)n2)cc1]

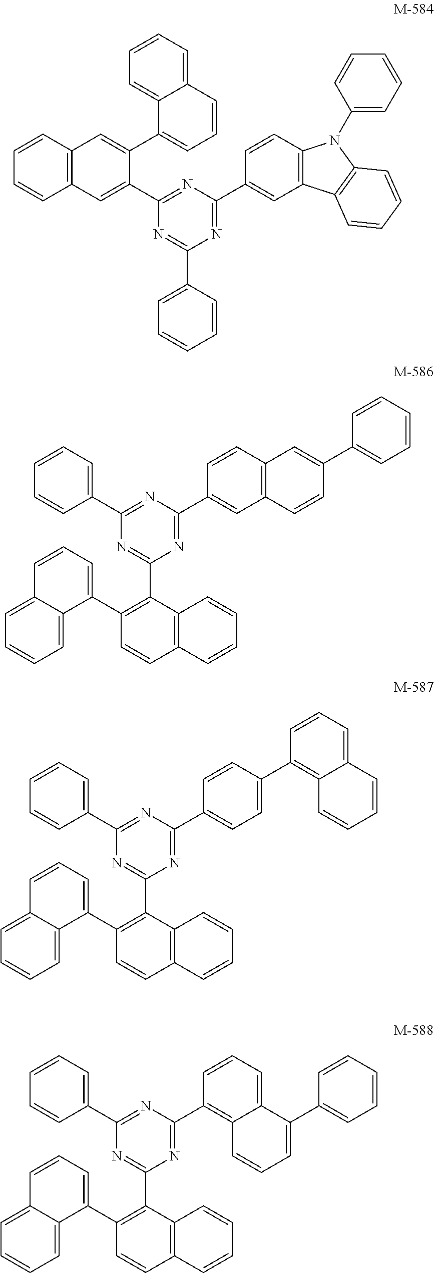 [SMILES: c1ccc(-c2nc(-c3ccc4c(c3)c3ccccc3n4-c3ccccc3)nc(-c3cc4ccccc4cc3-c3cccc4ccccc34)n2)cc1 ; c1ccc(-c2ccc3cc(-c4nc(-c5ccccc5)nc(-c5c(-c6cccc7ccccc67)ccc6ccccc56)n4)ccc3c2)cc1 ; c1ccc(-c2nc(-c3ccc(-c4cccc5ccccc45)cc3)nc(-c3c(-c4cccc5ccccc45)ccc4ccccc34)n2)cc1 ; c1ccc(-c2nc(-c3c(-c4cccc5ccccc45)ccc4ccccc34)nc(-c3cccc4c(-c5ccccc5)cccc34)n2)cc1]

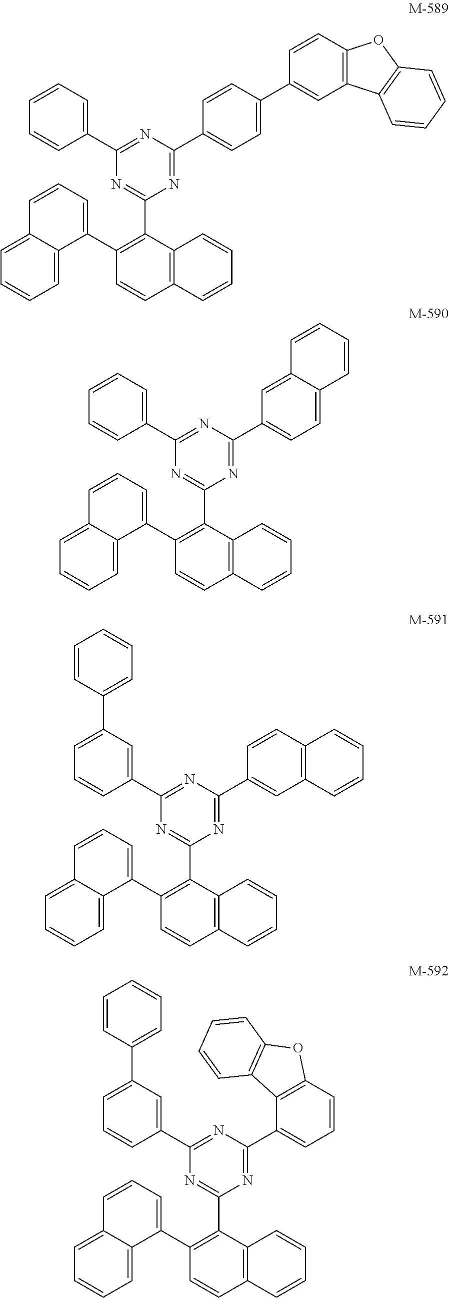

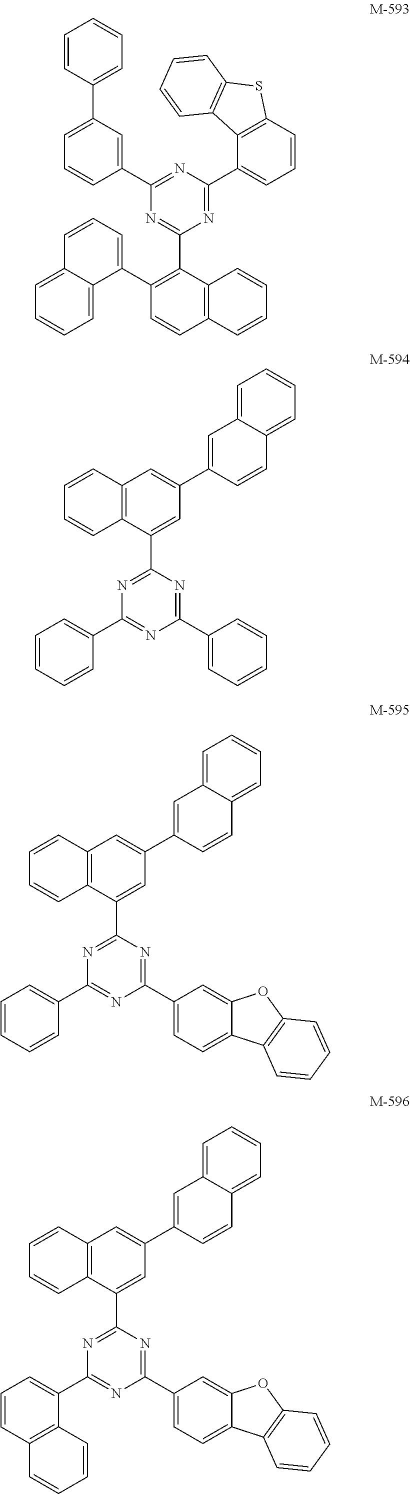

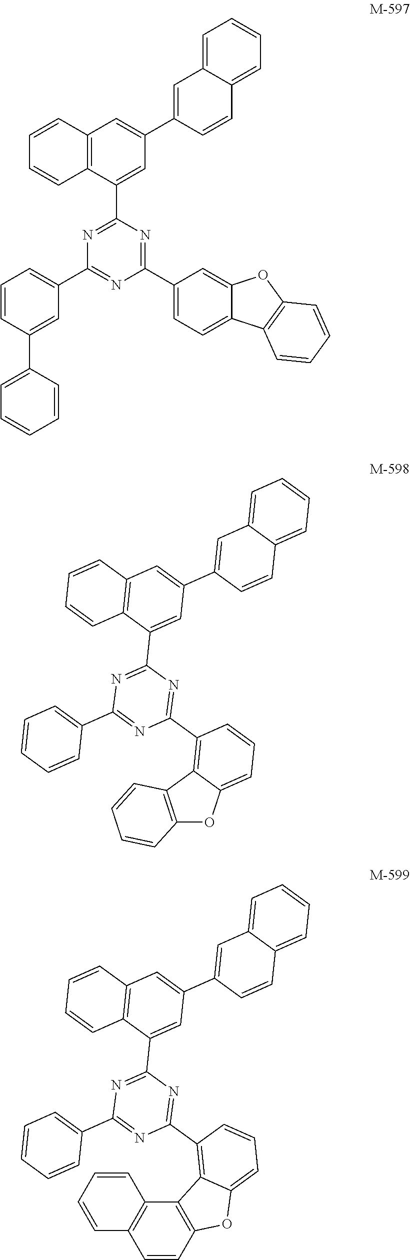 [SMILES: c1ccc(-c2cccc(-c3nc(-c4ccc5c(c4)oc4ccccc45)nc(-c4cc(-c5ccc6ccccc6c5)cc5ccccc45)n3)c2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cc(-c4ccc5ccccc5c4)cc4ccccc34)nc(-c3cccc4oc5ccc6ccccc6c5c34)n2)cc1]

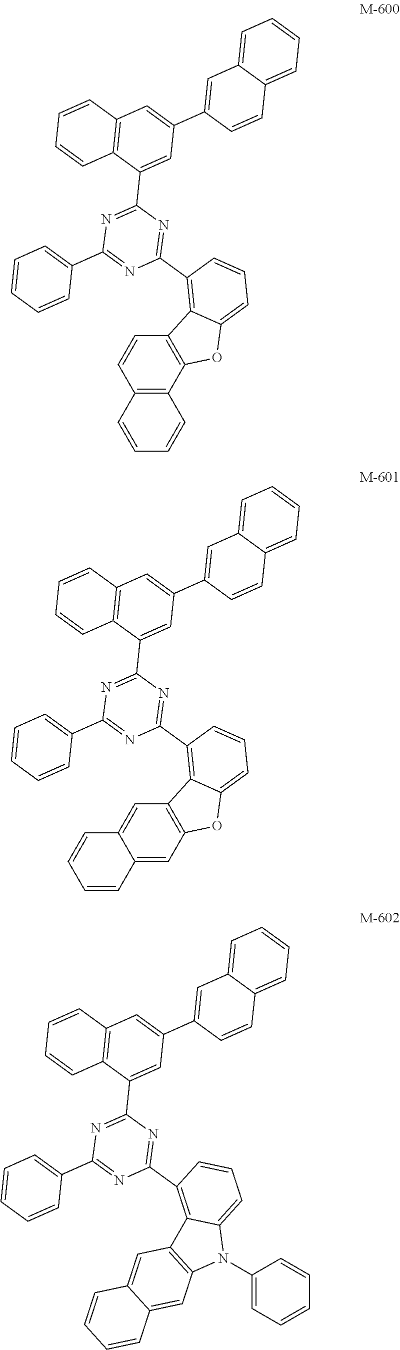 [SMILES: c1ccc(-c2nc(-c3cc(-c4ccc5ccccc5c4)cc4ccccc34)nc(-c3cccc4oc5c6ccccc6ccc5c34)n2)cc1 ; c1ccc(-c2nc(-c3cc(-c4ccc5ccccc5c4)cc4ccccc34)nc(-c3cccc4oc5cc6ccccc6cc5c34)n2)cc1 ; c1ccc(-c2nc(-c3cc(-c4ccc5ccccc5c4)cc4ccccc34)nc(-c3cccc4c3c3cc5ccccc5cc3n4-c3ccccc3)n2)cc1]

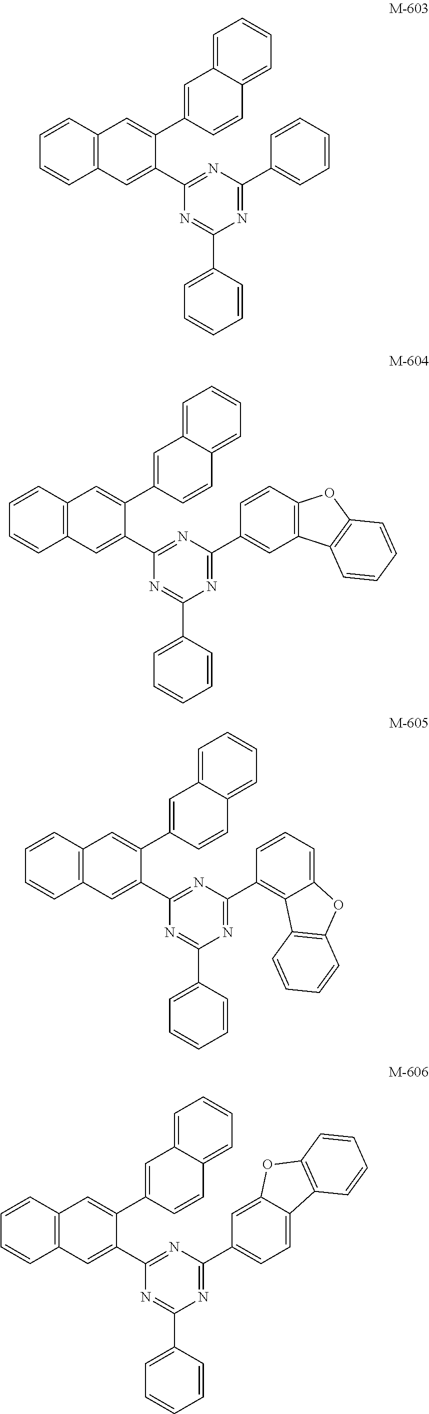 [SMILES: c1ccc(-c2nc(-c3ccccc3)nc(-c3cc4ccccc4cc3-c3ccc4ccccc4c3)n2)cc1 ; c1ccc(-c2nc(-c3ccc4oc5ccccc5c4c3)nc(-c3cc4ccccc4cc3-c3ccc4ccccc4c3)n2)cc1 ; c1ccc(-c2nc(-c3cc4ccccc4cc3-c3ccc4ccccc4c3)nc(-c3cccc4oc5ccccc5c34)n2)cc1 ; c1ccc(-c2nc(-c3ccc4c(c3)oc3ccccc34)nc(-c3cc4ccccc4cc3-c3ccc4ccccc4c3)n2)cc1]

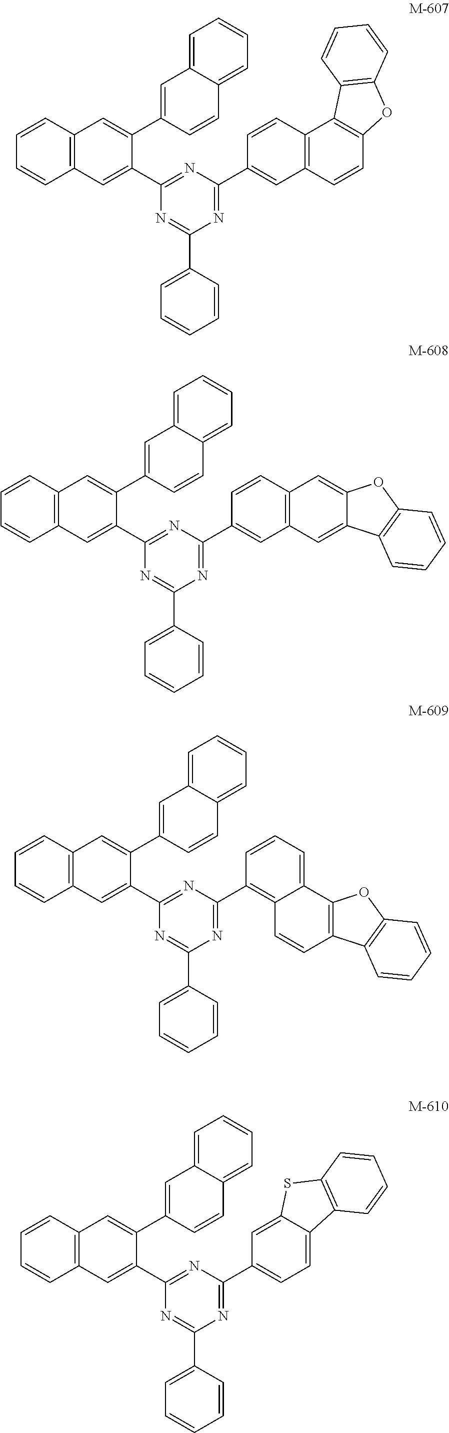

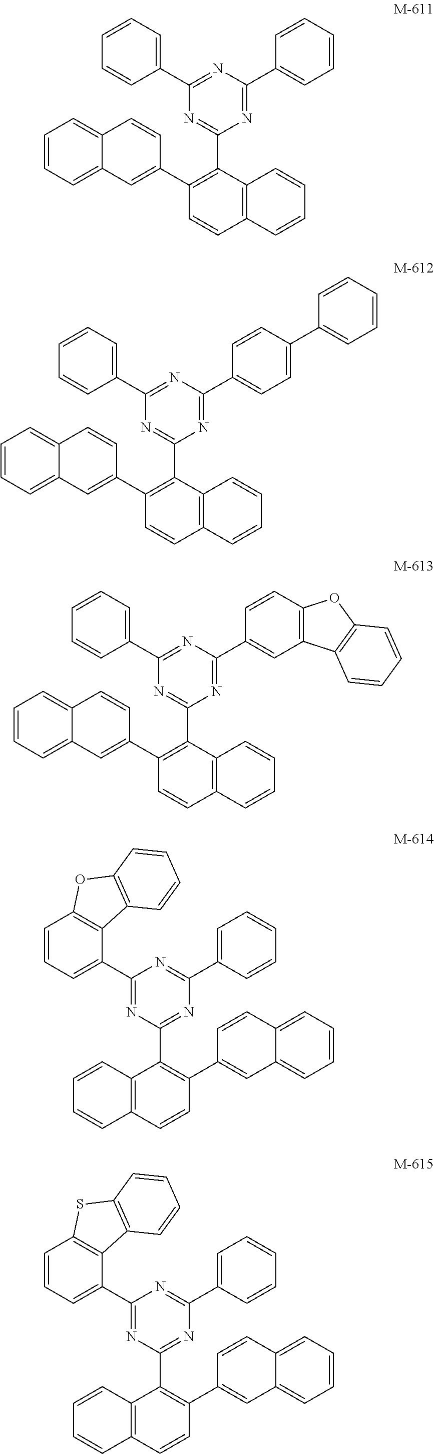 [SMILES: c1ccc(-c2nc(-c3ccccc3)nc(-c3c(-c4ccc5ccccc5c4)ccc4ccccc34)n2)cc1 ; c1ccc(-c2ccc(-c3nc(-c4ccccc4)nc(-c4c(-c5ccc6ccccc6c5)ccc5ccccc45)n3)cc2)cc1 ; c1ccc(-c2nc(-c3ccc4oc5ccccc5c4c3)nc(-c3c(-c4ccc5ccccc5c4)ccc4ccccc34)n2)cc1 ; c1ccc(-c2nc(-c3c(-c4ccc5ccccc5c4)ccc4ccccc34)nc(-c3cccc4oc5ccccc5c34)n2)cc1 ; c1ccc(-c2nc(-c3c(-c4ccc5ccccc5c4)ccc4ccccc34)nc(-c3cccc4sc5ccccc5c34)n2)cc1]

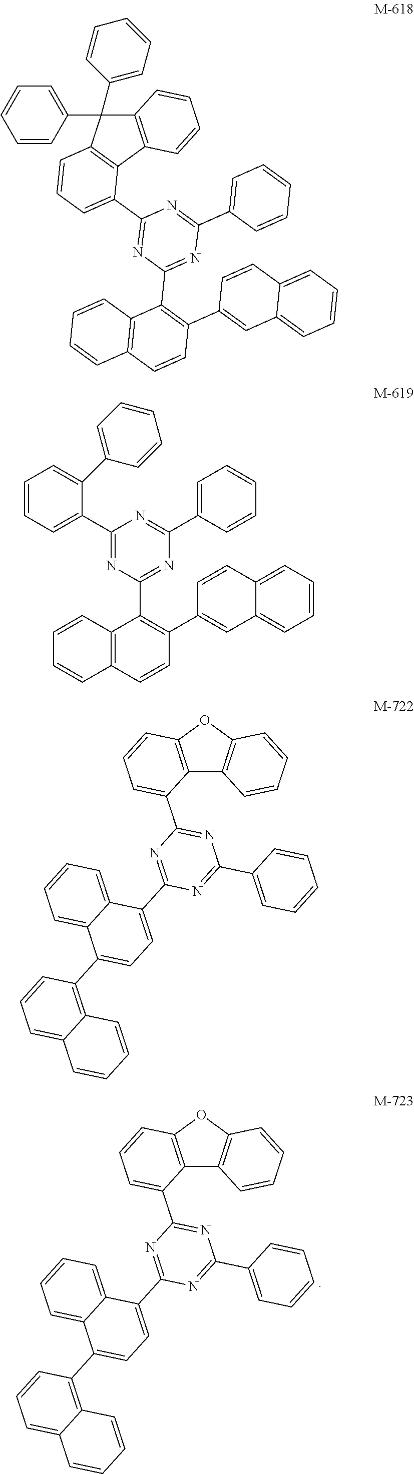

28. The organic material composition according to claim 14, wherein in the material composition, a mass ratio of the first compound to the second compound is 1:9-9:1.

29. The organic material composition according to claim 14, wherein in the material composition, the mass ratio of the first compound to the second compound is 2:8-8:2.

30. An organic electroluminescent host material composition, comprising the organic material composition according to claim 14.

31. An organic electroluminescent device, wherein the organic electroluminescent device comprises an anode and a cathode, and an organic layer disposed between the anode and the cathode, wherein the organic layer comprises the organic material composition according to claim 14.

32. The organic electroluminescent device according to claim 31, wherein the organic layer comprises a light-emitting layer, and the light-emitting layer comprises the organic material composition, wherein the organic material composition comprises a first compound and a second compound, wherein the first compound has a structure shown in Formula (1): 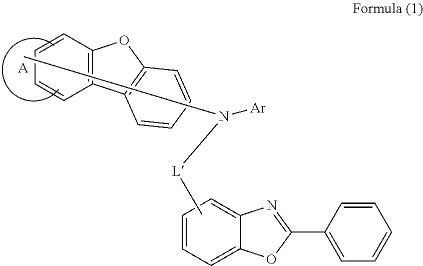
wherein ring A is a benzene ring;
Ar is selected from the group consisting of substituted or unsubstituted C6-C25 aryl;
L′ is selected from phenylene;
wherein the second compound has a structure shown in Formula (2): 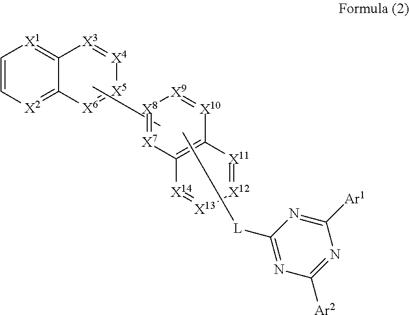
wherein X¹-X¹⁴ are each independently selected from N or CR, and R is selected from the group consisting of hydrogen, deuterium;
L is independently selected from a connecting bond;
Ar1 and Ar2 are each independently selected from unsubstituted C6-C25 aryl, unsubstituted C3-C20 heteroaryl;
the substituents in the substituted C6-C25 aryl is selected from the group consisting of deuterium and diphenylamine group.

33. An organic electroluminescent device, comprising the organic electroluminescent device according to claim 31.
